(19) United States
(12) Patent Application Publication
(10) Pub. No.: US 2024/0132480 A1
(43) Pub. Date: Apr. 25, 2024

(54) INHIBITORS OF MYCOBACTERIUM TUBERCULOSIS LIPOAMIDE DEHYDROGENASE

(71) Applicants: Cornell University, Ithaca, NY (US); Tri-Institutional Therapeutics Discovery Institute, Inc., New York, NY (US)

(72) Inventors: John Ginn, New York, NY (US); Shan SUN, New York, NY (US); Mayako Michino, New York, NY (US); Nigel Liverton, New York, NY (US); Rui Liang, New York, NY (US); Peter T. Meinke, New York, NY (US); David Huggins, New York, NY (US); Ruslana Bryk, Ithaca, NY (US); Carl F. Nathan, Ithaca, NY (US)

(21) Appl. No.: 18/270,621

(22) PCT Filed: Jan. 7, 2022
(86) PCT No.: PCT/US22/11588

§ 371 Date: Jun. 30, 2023

Related U.S. Application Data
(60) Provisional application No. 63/135,350, filed on Jan. 8, 2021.

(51) Int. Cl. C07D 403/12; A61P 31/06; C07D 209/08; C07D 209/34; C07D 209/42; C07D 231/56; C07D 401/12; C07D 401/14; C07D 405/12; C07D 405/14; C07D 413/12; C07D 413/14; C07D 417/12; C07D 471/04; C07D 513/04

(52) U.S. Cl. CPC C07D 403/12; A61P 31/06; C07D 209/08; C07D 209/34; C07D 209/42; C07D 231/56; C07D 401/12; C07D 401/14; C07D 405/12; C07D 405/14; C07D 413/12; C07D 413/14; C07D 417/12; C07D 471/04; C07D 513/04

(57) ABSTRACT

Disclosed are compounds for inhibiting lipoamide dehydrogenase (Lpd), and methods of treating tuberculosis.

### RELATED APPLICATIONS

This application claims the benefit of U.S. Provisional Application No. 63/135,350, filed Jan. 8, 2021, the contents of which are fully incorporated by reference herein.

### BACKGROUND

Tuberculosis (TB) infected 10 million and killed 1.5 million people in 2018. It remains a worldwide health crisis due to rising drug resistance and emerging risk factors, such as diabetes. Resistance of Mycobacterium tuberculosis (Mtb) to all first line anti-TB drugs is prevalent and calls for new strategies to develop effective therapeutics. The BPaL regimen recently approved by the FDA for the treatment of extensively drug resistant (XDR) and nonresponsive multidrug resistant (MDR) TB features are, for the first time in many decades, drugs against new Mtb targets and with novel modes of action. Nevertheless, more inhibitors against previously unexplored targets are urgently needed to sustain the TB drug pipeline and to shorten and diversify drug regimens, as resistance is already detected to components of BPaL.

### SUMMARY OF THE INVENTION

In certain aspects, the present disclosure provides compounds of formula I:

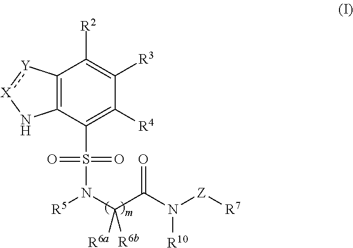

or a pharmaceutically acceptable salt thereof, wherein:
R², R³, and R⁴ independently are hydrogen, halogen, amino, hydroxyl, alkoxy, cyano, nitro, carbonyl, alkyl, alkenyl, alkynyl, cycloalkyl, heterocyclyl, aryl, or heteroaryl;
R⁵ and R¹⁰ are independently hydrogen, alkyl, cycloalkyl, heterocyclyl, aryl, or heteroaryl;
each R^{6a} and R^{6b} independently is hydrogen, halogen, amino, hydroxyl, alkoxy, cyano, nitro, alkyl, alkenyl, alkynl, cycloalkyl, heterocyclyl, aryl, or heteroaryl; or R^{6a} and R^{6b} complete a cycloalkyl, heterocyclyl, or an oxo group; or R⁵, R^{6a}, and the intervening carbon and nitrogen atoms complete a 3- to 6-membered heterocyclyl;
R⁷ is alkyl, alkenyl, alkynyl, cycloalkyl, heterocyclyl, aryl, or heteroaryl;
[symbol] is a single bond or a double bond; wherein when [symbol] is a double bond, X is N or CR⁸, and Y is N or CR¹; when [symbol] is a single bond, X is NR⁵, C(R⁸)₂, or C(═O) and Y is NR⁵, C(R¹)₂, or C(═O);
R¹ and R⁸ independently are hydrogen, halogen, amino, hydroxyl, alkoxy, cyano, nitro, alkyl, alkenyl, alkynyl, cycloalkyl, heterocyclyl, aryl, or heteroaryl;
Z is —C(R⁹)₂— or a bond;
each R⁹ is independently H or alkyl; and
m is an integer from 1 to 3.

In certain aspects, the present disclosure provides methods of inhibiting the growth or killing of Mycobacterium tuberculosis in vitro, comprising contacting Mycobacterium tuberculosis with a compound disclosed herein.

In certain aspects, the present disclosure provides methods of treating tuberculosis, comprising administering to a subject in need thereof a compound disclosed herein.

### BRIEF DESCRIPTION OF THE DRAWINGS

FIGS. 1A-1D show compound C is a potent, slow binding inhibitor of Mtb Lpd. Inhibition of Lpd activity is time-dependent (FIG. 1A). The IC₅₀ depends on Lpd concentration (FIG. 1B). The first order rate constant of inactivation increases with inhibitor concentration (FIG. 1C). Recovery of Lpd activity upon dilution of the Lpd-C complex (FIG. 1D). In FIG. 1A, Lpd was used at 66 nM and compound C concentrations are indicated in nM. In FIG. 1B, Lpd concentrations are indicated in nM; the inset shows the plot of [Lpd] vs calculated IC₅₀. In FIG. 1C, k_{obs} values were determined from the first order association fit represented by a solid line in A. Substrate lipoamide concentration was 75 μM. In FIG. 1D Lpd (10 μM) was preincubated with the indicated μM concentrations of compound C for 30 min at RT, diluted 500-fold into the reaction mix with 75 μM lipoamide and monitored for TNB formation over time.

FIG. 2 shows the recovery of Lpd activity after preincubation with compound C. TNB formation was recorded over time upon 500× dilution of the preincubation of 10 μM WT Mtb Lpd with the indicated concentrations of compound C for 30 min at RT. Representative of 3 independent experiments is presented. Data was fitted to the integrated rate equation P=Vₛ*t+(V₀−Vₛ)*((1−e^{-koff*t})/k_{off}), where P is TNB formation at A₄₁₂, Vₛ is steady state velocity, V₀ is initial velocity, k_{off} is dissociation rate, t_{1/2}=1/k_{off}.

FIGS. 3A-C shows compound C is a readily reversible inhibitor of human Lpd. (FIG. 3A) Activity of human Lpd (280 nM) was monitored over time in the presence of indicated μM concentrations of compound C. (FIG. 3B) IC₅₀ values were calculated at variable Lpd (75, 124, 187 and 280 nM) and plotted against [Lpd]. (FIG. 3C) Human Lpd (10 μM) was preincubated with indicated μM concentrations of compound C for 30 min at RT, diluted 125-fold into the reaction mix with 75 μM lipoamide and monitored for TNB formation over time.

FIGS. 4A-B shows compound C is a rapid equilibrium inhibitor of the Mtb Lpd R93A mutant. (FIG. 4A) Progress curves for Lpd R93A (200 nM) at indicated nM concentrations of compound C. Reaction was initiated by enzyme addition. (FIG. 4B) Lpd R93A (10 μM) was preincubated with indicated concentrations of compound C (in μM), diluted 125-fold into the reaction mix with 75 μM lipoamide and monitored for TNB formation over time.

FIG. 5 shows ITC data for Mtb WT, R93A Lpd and human Lpd interaction with compound C. All proteins were at 20 μM, compound C at 250 μM in 25 mM potassium phosphate buffer, pH 7.0. Representative of two independent runs is shown.

FIG. 6 are fitted isotherms and residuals for compound C binding to Mtb WT, R93A Lpd and human Lpd protein in the absence or presence of 100 μM NADH. All proteins were at 20 μM, compound C at 250 μM in 25 mM potassium phosphate buffer, pH 7.0. A Stoichiometric Equilibria model was used to fit the data. Experimental data, black; fitted data, red. K_{d} numbers provided in Table 4. Fitted isotherms are for data provided in FIG. 2.

FIG. 7 shows thermal footprints of compound C binding to Mtb WT Lpd in the absence or presence of 100 μM NADH in 25 mM potassium phosphate (KPi), pH 7.0 (top panels) or 20 mM triethanolamine (TEA), pH 7.8 (bottom panels).

FIG. 8 is a SPR analysis of compound C binding to Mtb and human Lpd. Mtb Lpd data was fitted to two state model, human data was fitted to 1:1 Stoichiometric binding model. Representative of two independent runs is shown. Black lines, fit curves; colored lines, experimental data.

FIGS. 9A-C shows compound C recapitulates Mtb Δlpd phenotypes. (FIG. 9A) WT Mtb was exposed to pH 5.5 or pH 5.5 plus 3 mM NaNO₂ in the presence of indicated concentrations of C for 4 days and plated on agar to enumerate colony-forming units (CFU) of surviving bacteria. (FIG. 9B) Mouse BMDM were infected with WT Mtb (multiplicity of infection (MOI)=0.1) and exposed to indicated M concentrations of compound C. At the indicated timepoints BMDM were lysed and CFU determined. (FIG. 9C) As in B but BMDM were activated with 10 ng/ml IFNγ for 24 h prior to infection. Data are from one experiment representative of two independent experiments.

### DETAILED DESCRIPTION OF THE INVENTION

Lipoamide dehydrogenase (Lpd) fulfills multiple metabolic functions and a unique antioxidant function in Mtb. It is a component of the pyruvate, alpha-ketoglutarate and branched chain ketoacid dehydrogenase complexes involved in the breakdown of ketoacids coupled to the production of energy-rich acyl-CoA intermediates and NADH. In the reverse direction, Lpd supplies reducing equivalents from NADH through the lipoylated E2 cores of metabolic complexes to the thioredoxin-like adaptor AhpD and the peroxiredoxin AhpC to detoxify reactive nitrogen and oxygen intermediates. Mtb lacking Lpd fails to grow on carbohydrates as a sole carbon source in vitro, cannot establish TB infection in mice, is highly susceptible to reactive nitrogen intermediates, and accumulates a ˜100-fold excess of intracellular pyruvate, alanine, valine, leucine, isoleucine and their corresponding ketoacids³. The dependence of Mtb on Lpd for virulence and persistence provides genetic validation of Lpd as a target, but chemical validation remains to be achieved.

Bacterial enzymes with human homologs are often viewed as unattractive targets due to possible host toxicity. Although the three-dimensional structures of the mycobacterial and human enzymes align closely, mycobacterial Lpd is only 33% identical to the human homolog with pronounced differences in their substrate binding sites. This has allowed us to discover Lpd inhibitors with high species selectivity.

In certain aspects, the present disclosure provides compounds of formula I:

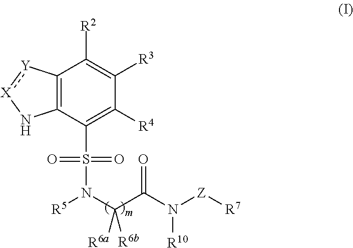

or a pharmaceutically acceptable salt thereof, wherein:
R², R³, and R⁴ independently are hydrogen, halogen, amino, hydroxyl, alkoxy, cyano, nitro, carbonyl, alkyl, alkenyl, alkynyl, cycloalkyl, heterocyclyl, aryl, or heteroaryl;
R⁵ and R¹⁰ are independently hydrogen, alkyl, cycloalkyl, heterocyclyl, aryl, or heteroaryl;
each R^{6a} and R^{6b} independently is hydrogen, halogen, amino, hydroxyl, alkoxy, cyano, nitro, alkyl, alkenyl, alkynl, cycloalkyl, heterocyclyl, aryl, or heteroaryl; or R^{6a} and R^{6b} complete a cycloalkyl, heterocyclyl, or an oxo group; or R⁵, R^{6a}, and the intervening carbon and nitrogen atoms complete a 3- to 6-membered heterocyclyl;
R⁷ is alkyl, alkenyl, alkynyl, cycloalkyl, heterocyclyl, aryl, or heteroaryl;
[symbol] is a single bond or a double bond; wherein when [symbol] is a double bond, X is N or CR⁸, and Y is N or CR¹; when [symbol] is a single bond, X is NR⁵, C(R⁸)₂, or C(═O) and Y is NR⁵, C(R¹)₂, or C(═O);
R¹ and R⁸ independently are hydrogen, halogen, amino, hydroxyl, alkoxy, cyano, nitro, alkyl, alkenyl, alkynyl, cycloalkyl, heterocyclyl, aryl, or heteroaryl;
Z is —C(R⁹)₂— or a bond;
each R⁹ is independently H or alkyl; and
m is an integer from 1 to 3.

In certain embodiments, the present disclosure provides compounds of formula I:

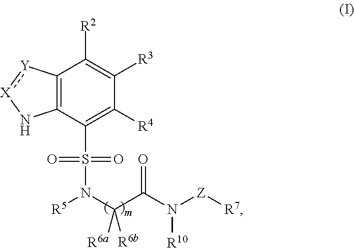

and pharmaceutically acceptable salts thereof, wherein
R², R³, and R⁴ independently are hydrogen, halogen, amino, hydroxyl, alkoxy, cyano, nitro, alkyl, alkenyl, alkynl, cycloalkyl, heterocyclyl, aryl, or heteroaryl;
R⁵ and R¹⁰ are independently hydrogen, alkyl, cycloalkyl, heterocyclyl, aryl, or heteroaryl; each R^{6a} and R^{6b} independently is hydrogen, halogen, amino, hydroxyl, alkoxy, cyano, nitro, alkyl, alkenyl, alkynl, cycloalkyl, heterocyclyl, aryl, or heteroaryl; or R^{6a} and R^{6b} complete a cycloalkyl, heterocyclyl, or an oxo group; or R⁵, R^{6a}, and the intervening carbon and nitrogen atoms complete a 3- to 6-membered heterocyclyl;
R⁷ is alkyl, cycloalkyl, heterocyclyl, aryl, or heteroaryl;
[symbol] is a single bond or a double bond; wherein when [symbol] is a double bond, X is N or CR⁸, and Y is N or CR¹; when [symbol] is a single bond, X is NR⁵, C(R⁸)₂, or C(═O) and Y is NR⁵, C(R¹)₂, or C(═O);
R¹ and R^{B} independently are hydrogen, halogen, amino, hydroxyl, alkoxy, cyano, nitro, alkyl, alkenyl, alkynl, cycloalkyl, heterocyclyl, aryl, or heteroaryl;
Z is CH₂ or a bond; and
m is an integer from 1 to 3.

In certain embodiments, the compound is a compound of formula Ia

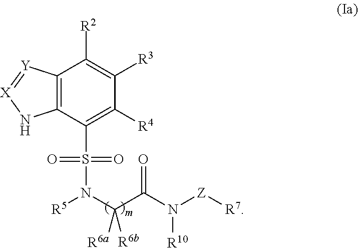

In certain embodiments, X is CR⁸. In certain such embodiments, R⁸ is hydrogen, halogen, amino, alkoxy, cyano, nitro, alkyl (e.g., alkyl-alkoxy such as methylene alkoxy or alkyl-aminoalkyl such as methylene-aminomethyl), cycloalkyl, or heterocyclyl. In further embodiments, R⁸ is hydrogen, halogen, cyano, or C₁₋₆ alkyl. In yet further embodiments, R⁸ is hydrogen, cyano, or methyl.

In certain preferred embodiments, X is N.

In certain preferred embodiments, Y is CR¹. In certain such embodiments, R¹ is hydrogen, halogen, alkyl (e.g., alkyl-alkoxy such as methylene alkoxy or alkyl-aminoalkyl such as methylene-aminomethyl), alkenyl, alkynl, cycloalkyl, heterocyclyl, aryl, or heteroaryl. In further embodiments, R¹ is hydrogen, halogen, or C₁₋₆ alkyl. In yet further embodiments, R¹ is hydrogen, methyl, halogen, cyano, methylamino methyl, or methoxy methyl. In certain embodiments, R¹ is hydrogen.

In certain embodiments, Y is N.

In certain embodiments, R² is hydrogen, halogen, alkyl (e.g., haloalkyl), alkenyl, alkynl, cycloalkyl, heterocyclyl, aryl, or heteroaryl. In certain, embodiments, R² is hydrogen, halogen, or C₁₋₆ alkyl. In certain preferred embodiments, R² is hydrogen or fluoro.

In certain, embodiments, R³ is halogen, cyano, alkyl (e.g., haloalkyl), cycloalkyl, or heterocyclyl. In certain such embodiments, R³ is halogen, cyano, C₁₋₆ alkyl, C₃₋₁₀ cycloalkyl, or C₁₋₆ haloalkyl. In yet further embodiments, R³ is cyano, methyl, chloro, bromo, cyclopropyl, or difluoromethyl. In certain preferred embodiments, R³ is halogen or C₁₋₆ alkyl. In even more preferred embodiments, R³ is methyl.

In certain, embodiments, R⁴ is hydrogen, halogen, alkyl (e.g., haloalkyl), cycloalkyl, or heterocyclyl. In certain such embodiments, R⁴ is hydrogen or halogen. In certain preferred embodiments, R⁴ is hydrogen or fluoro.

In certain embodiments, the compound is a compound of formula IIa

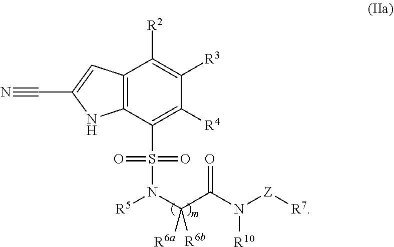

In certain, embodiments, the compound of formula I is a compound of formula I-1 or I-2

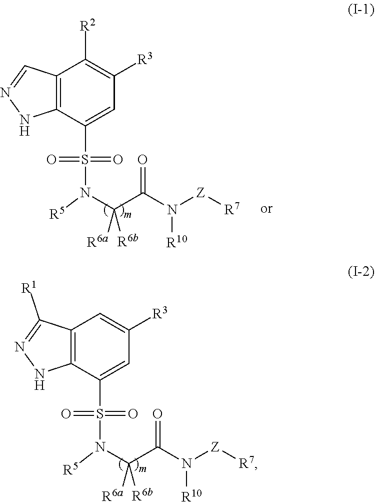

wherein m is 1 or 2.

In certain embodiments, R⁵ and R¹⁰ are independently hydrogen, alkyl, cycloalkyl, or heterocyclyl. In certain such embodiments, R is hydrogen, C₁₋₆ alkyl or 3- to 10-membered heterocyclyl. In certain preferred embodiments, R is hydrogen, methyl, ethyl, isopropyl, or oxetanyl. In certain embodiments, R⁵ is C₁₋₆ alkyl or 3- to 10-membered heterocyclyl. In certain preferred embodiments, R⁵ is methyl, ethyl, isopropyl, or oxetanyl.

In certain preferred embodiments, R¹⁰ is hydrogen.

In certain embodiments, R^{6a} and R^{6b} independently are hydrogen or alkyl (such as C₁₋₆ alkyl, e.g., methyl).

In certain embodiments, R^{6a} and R^{6b} are taken together to form a C₃₋₁₀ cycloalkyl, 3- to 10-membered heterocycle, or an oxo group.

In certain embodiments, R⁵, R^{6a}, and the intervening carbon and nitrogen atoms are taken together to form a 3- to 6-membered heterocycle.

In certain embodiments, m is 1 or 2, preferably 1.

In certain embodiments, the compound of formula I is a compound of formula I-1-a, I-1-b, I-1-c, I-2-a, I-2-b, or I-2-c:

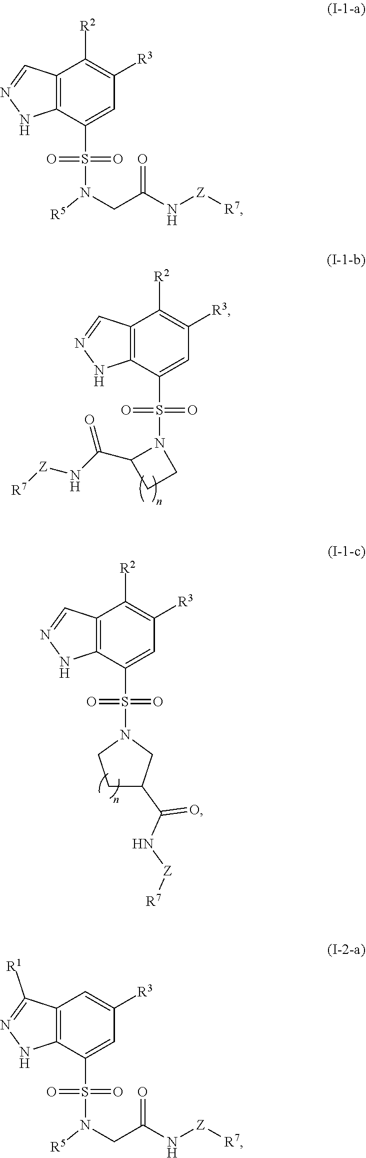

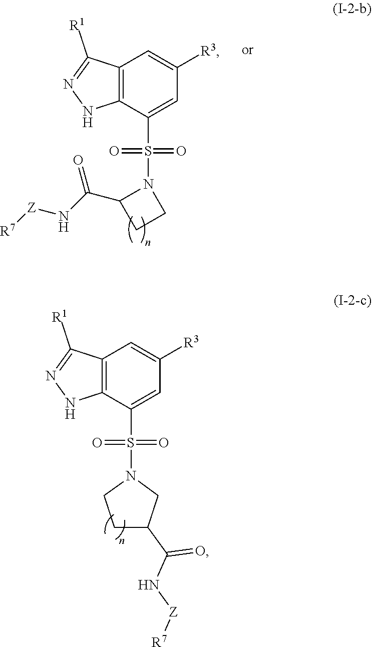

In certain embodiments, n is 1 or 2.

In certain embodiments, the compound of formula I is a compound of formula I-3

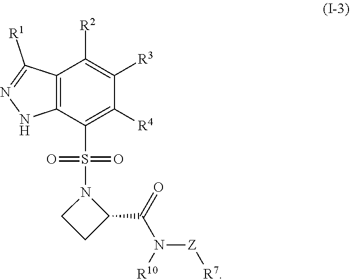

In certain embodiments, the compound of formula I is a compound of formula II-1-a

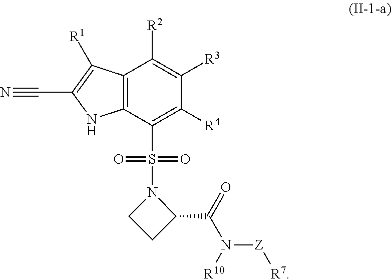

In certain embodiments, Z is a bond, such that R⁷ and the —N(H)— are directly connected by a single bond.

In certain embodiments, Z is C(R⁹)₂, where each R⁹ is independently alkyl (e.g., methyl) or hydrogen. In certain preferred embodiments, Z is CH₂.

In certain embodiments, R⁷ is C₁₋₆ alkyl, C₃₋₁₀ cycloalkyl or 3- to 10-membered heterocyclyl, C₆₋₁₀ aryl or 5- to 10-membered heteroaryl. In certain such embodiments, R⁷ is substituted with C₁₋₃ alkyl, cyclopropyl, C₁₋₃ alkoxy, halogen, oxo, cyano, phenyl, morpholino, piperidine, tetrahydropyran, or oxetane. In other such embodiments, R⁷ is unsubstituted.

In certain embodiments, R⁷ is C₁₋₆ alkyl, C₆₋₁₀ aryl or 5- to 10-membered heterocyclyl. In certain preferred embodiments, R⁷ is 6 aryl or 5- to 6-membered heterocyclyl. In certain embodiments, R⁷ is optionally substituted with C₁₋₃ alkyl, cyclopropyl, C₁₋₃ alkoxy, halogen, oxo, cyano, phenyl, morpholino, piperidine, tetrahydropyran, or oxetane.

In certain embodiments, R⁷ is cyclohexyl, tetrahydropyran, piperidine, piperidin-2-one, phenyl, pyridyl, pyridine-2-one, thiazole, oxazole, triazole, benzoxazole, benzthiazole, quinoline, 3,4-dihydro-2H-benzo[b][1,4]oxazine, 2,3-dihydrobenzo[b][1,4]dioxine, 4,5,6,7-tetrahydrothiazolo[5,4-c]pyridine, 4,5,6,7-tetrahydrothiazolo[4,5-c]pyridine, 2,3-dihydro-1H-indene, 6,7-dihydro-5H-cyclopenta[c]pyridine, imidazo[1,5-a]pyridine, [1,2,4]triazolo[4,3-a]pyridine, pyrazolo[1,5-a]pyridine, pyrazolo[1,5-a]pyridine, indolin-2-one. In certain preferred embodiments, R⁷ is phenyl or piperidin-2-one.

In certain embodiments, R⁷ is alkyl (e.g., C₁-C₆ alkyl), alkenyl (e.g., C₂-C₆ alkenyl), or alkynyl (e.g., C₂-C₆ alkynyl), each of which may be optionally substituted with groups such as aryl, cycloalkyl, or cyano. For example, when R⁷ is alkynyl, R⁷ can be propargyl,

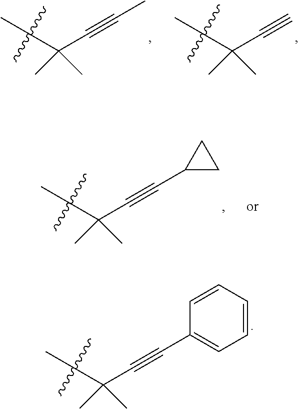

For example when R⁷ is alkyl, R⁷ can be

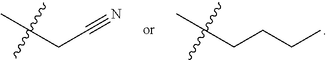

In certain embodiments, R⁷

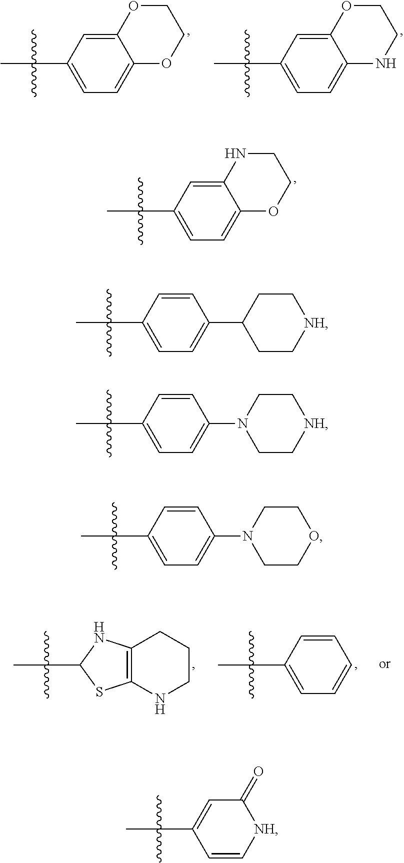

each of which may be optionally substituted with C₁₋₆ alkyl, cyclopropyl, C₁₋₃ alkoxy, halogen, oxo, cyano, phenyl, amido, or heterocyclyl such as morpholino, piperidine, piperazine, piperidin-2-one, tetrahydropyran, and oxetane. In certain preferred embodiments, R⁷ is

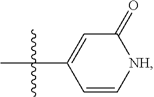 [SMILES: *c1cc[nH]c(=O)c1]

which may be optionally substituted. In certain preferred embodiments, R⁷ is

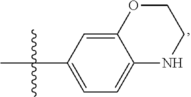 [SMILES: *c1ccc2c(c1)OCCN2]

which may be optionally substituted.

In certain embodiments, the compound is selected from:

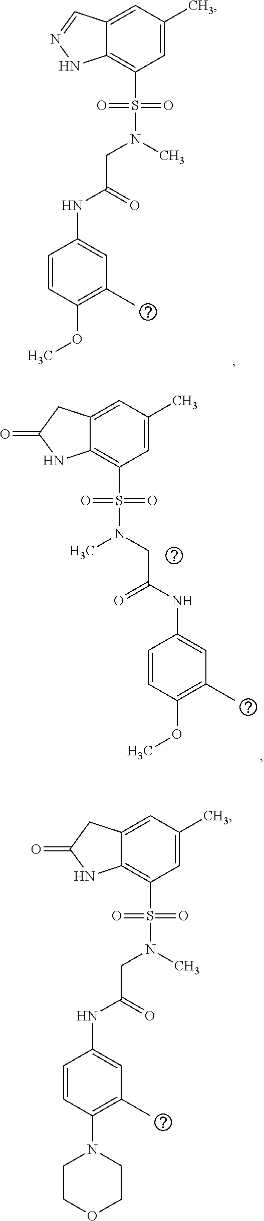 [SMILES: COc1ccc(NC(=O)CN(C)S(=O)(=O)c2cc(C)cc3cn[nH]c23)cc1C ; COc1ccc(NC(=O)CN(C)S(=O)(=O)c2cc(C)cc3c2NC(=O)C3)cc1C ; Cc1cc2c(c(S(=O)(=O)N(C)CC(=O)Nc3ccc(N4CCOCC4)c(C)c3)c1)NC(=O)C2]

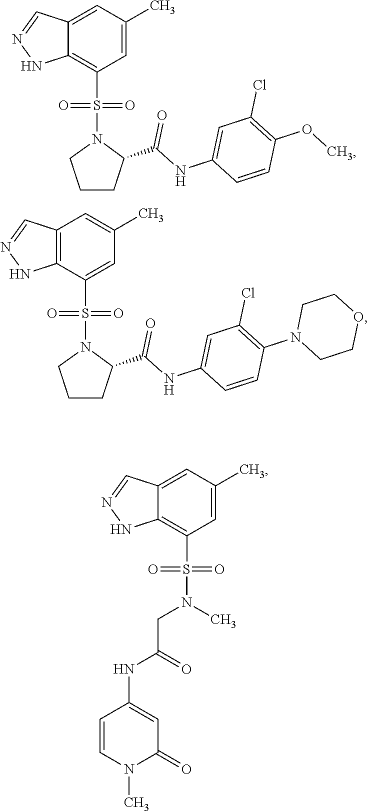

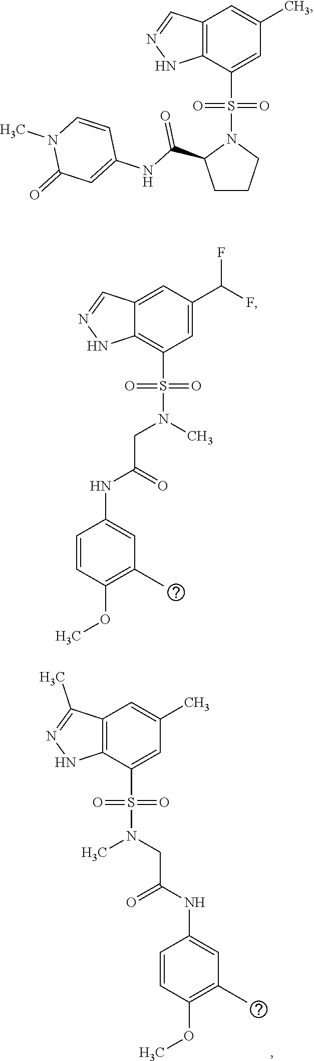

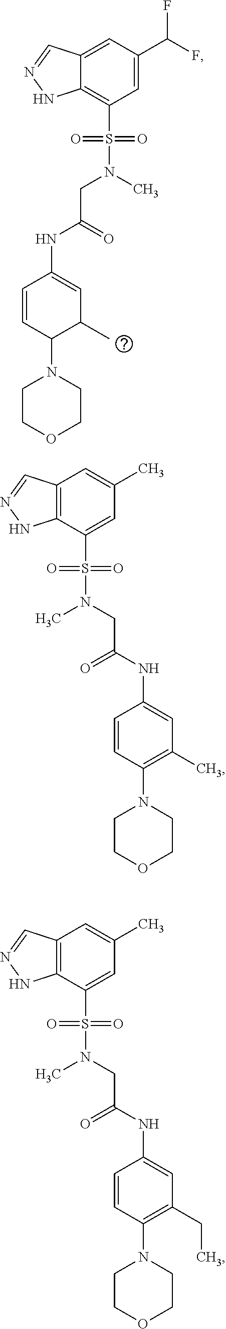

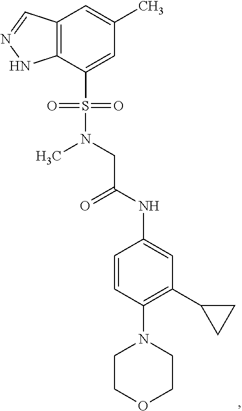 [SMILES: Cc1cc(S(=O)(=O)N(C)CC(=O)Nc2ccc(N3CCOCC3)c(C3CC3)c2)c2[nH]ncc2c1]

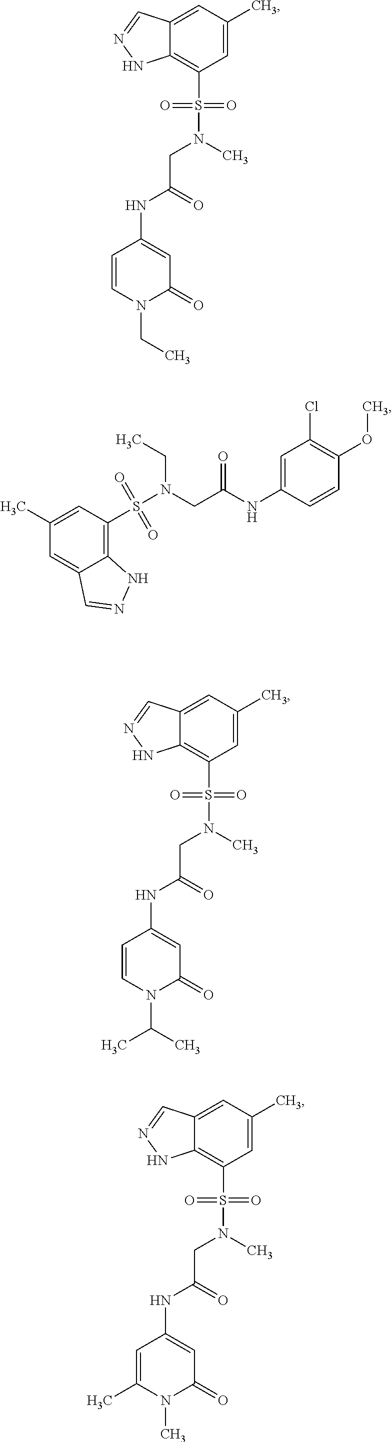 [SMILES: CCn1ccc(NC(=O)CN(C)S(=O)(=O)c2cc(C)cc3cn[nH]c23)cc1=O ; CCN(CC(=O)Nc1ccc(OC)c(Cl)c1)S(=O)(=O)c1cc(C)cc2cn[nH]c12 ; Cc1cc(S(=O)(=O)N(C)CC(=O)Nc2ccn(C(C)C)c(=O)c2)c2[nH]ncc2c1 ; Cc1cc(S(=O)(=O)N(C)CC(=O)Nc2cc(C)n(C)c(=O)c2)c2[nH]ncc2c1]

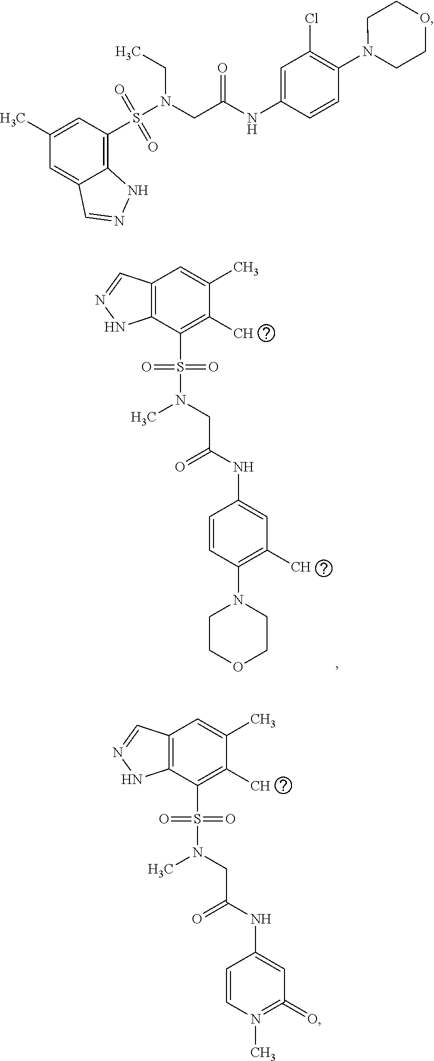

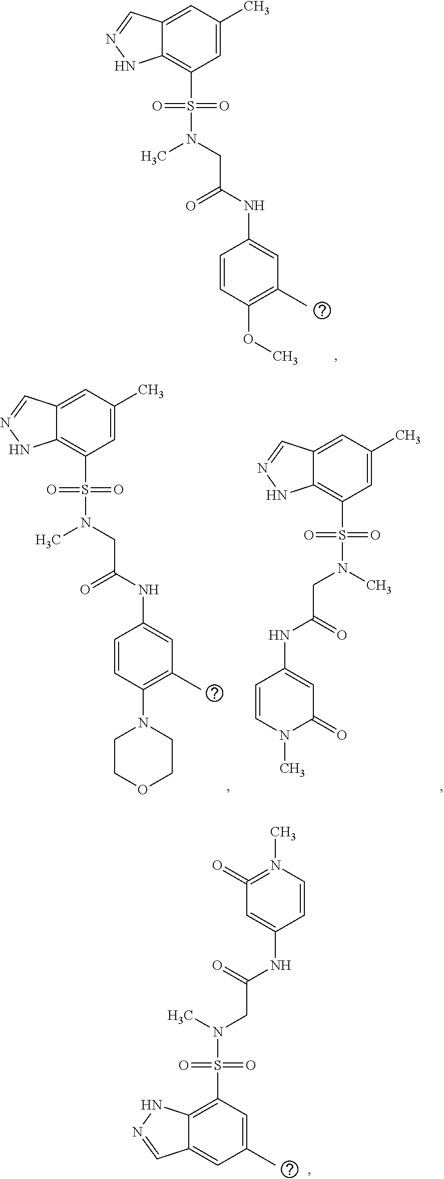

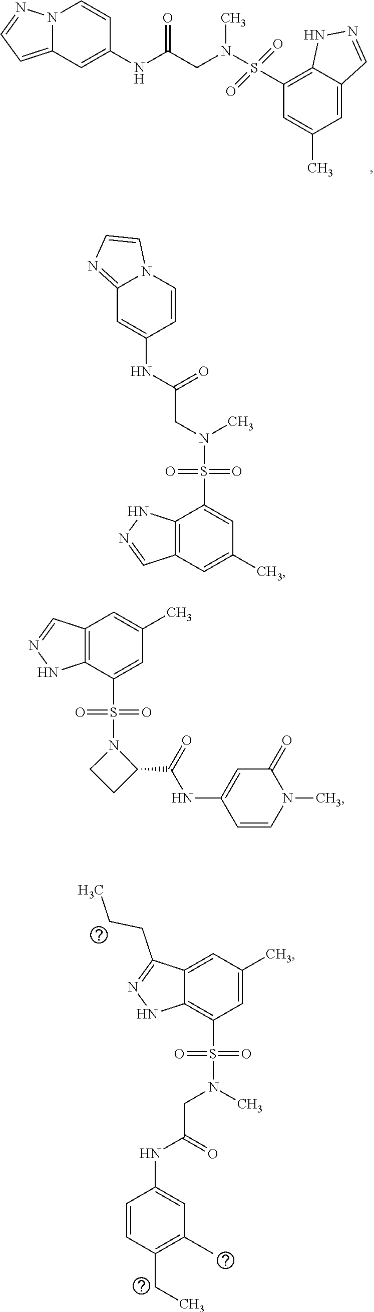 [SMILES: Cc1cc(S(=O)(=O)N(C)CC(=O)Nc2ccn3nccc3c2)c2[nH]ncc2c1 ; Cc1cc(S(=O)(=O)N(C)CC(=O)Nc2ccn3ccnc3c2)c2[nH]ncc2c1 ; Cc1cc(S(=O)(=O)N2CC[C@H]2C(=O)Nc2ccn(C)c(=O)c2)c2[nH]ncc2c1 ; CCCc1n[nH]c2c(S(=O)(=O)N(C)CC(=O)Nc3ccc(CC)c(C)c3)cc(C)cc12]

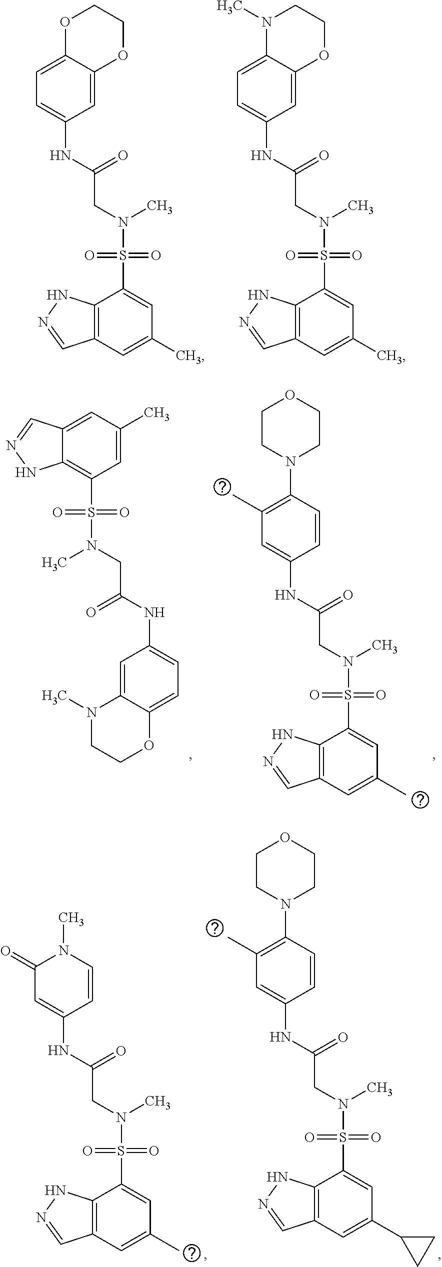

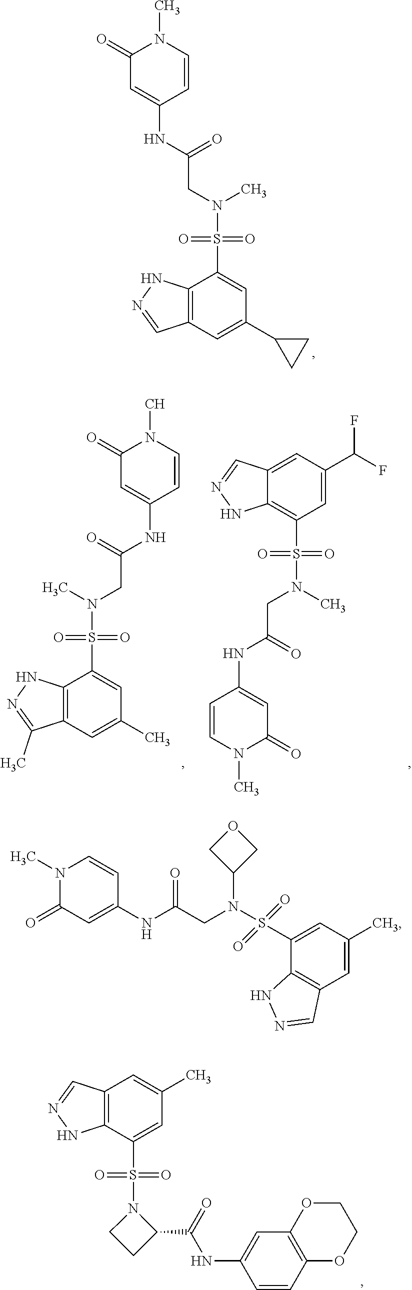

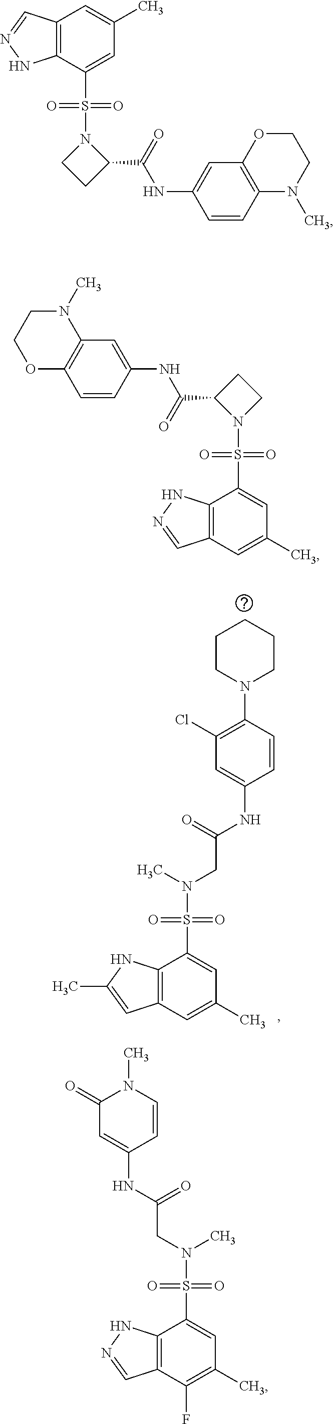

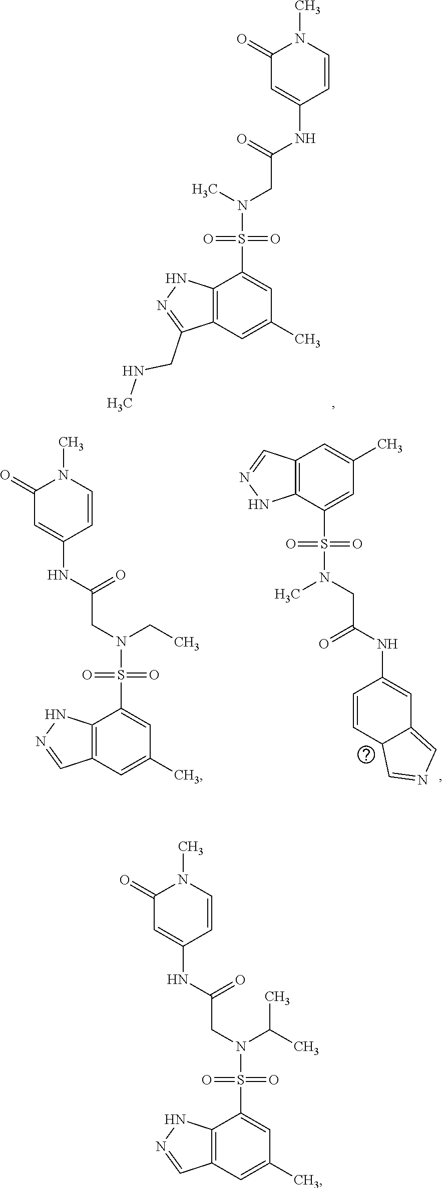 [SMILES: CNCc1n[nH]c2c(S(=O)(=O)N(C)CC(=O)Nc3ccn(C)c(=O)c3)cc(C)cc12 ; CCN(CC(=O)Nc1ccn(C)c(=O)c1)S(=O)(=O)c1cc(C)cc2cn[nH]c12 ; Cc1cc(S(=O)(=O)N(C)CC(=O)NC2=CC3=CN=CC3C=C2)c2[nH]ncc2c1 ; Cc1cc(S(=O)(=O)N(CC(=O)Nc2ccn(C)c(=O)c2)C(C)C)c2[nH]ncc2c1]

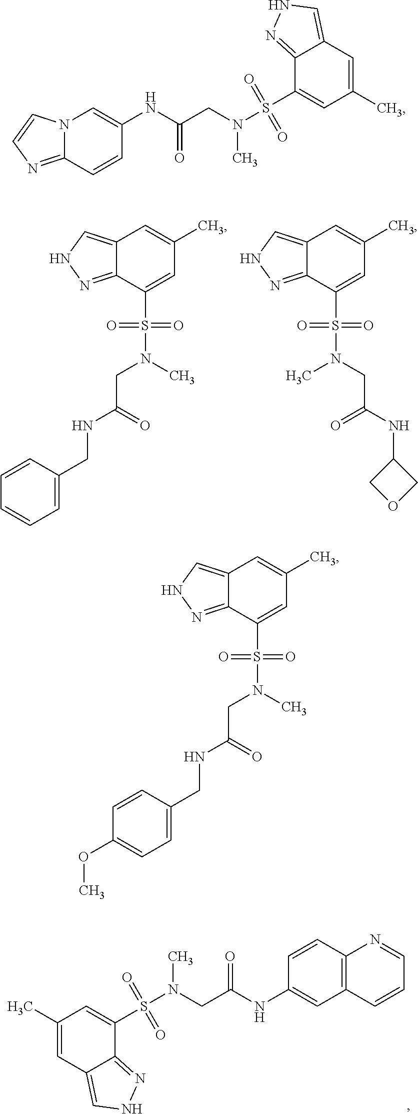 [SMILES: Cc1cc(S(=O)(=O)N(C)CC(=O)Nc2ccc3nccn3c2)c2n[nH]cc2c1 ; Cc1cc(S(=O)(=O)N(C)CC(=O)NCc2ccccc2)c2n[nH]cc2c1 ; Cc1cc(S(=O)(=O)N(C)CC(=O)NC2COC2)c2n[nH]cc2c1 ; COc1ccc(CNC(=O)CN(C)S(=O)(=O)c2cc(C)cc3c[nH]nc23)cc1 ; Cc1cc(S(=O)(=O)N(C)CC(=O)Nc2ccc3ncccc3c2)c2n[nH]cc2c1]

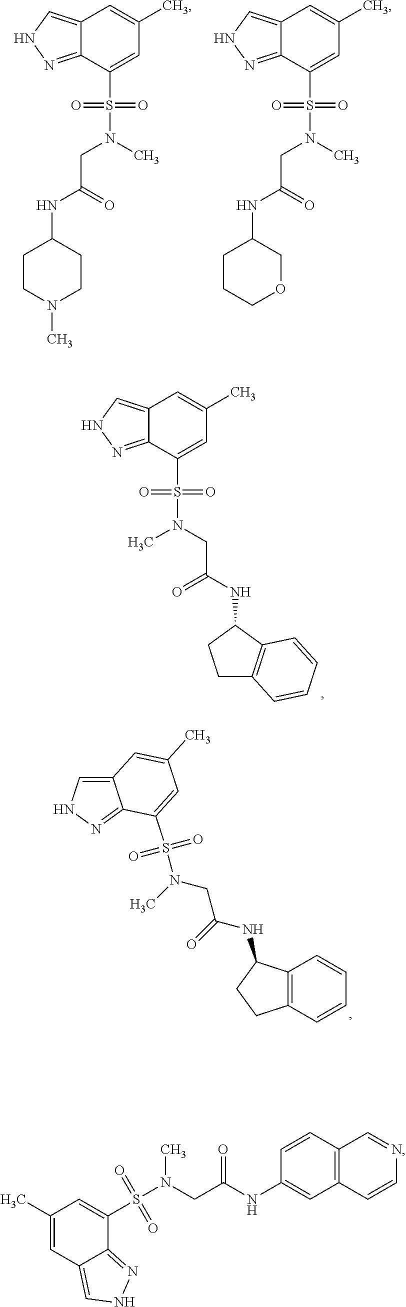 [SMILES: Cc1cc(S(=O)(=O)N(C)CC(=O)NC2CCN(C)CC2)c2n[nH]cc2c1 ; Cc1cc(S(=O)(=O)N(C)CC(=O)NC2CCCOC2)c2n[nH]cc2c1 ; Cc1cc(S(=O)(=O)N(C)CC(=O)N[C@H]2CCc3ccccc32)c2n[nH]cc2c1 ; Cc1cc(S(=O)(=O)N(C)CC(=O)N[C@@H]2CCc3ccccc32)c2n[nH]cc2c1 ; Cc1cc(S(=O)(=O)N(C)CC(=O)Nc2ccc3cnccc3c2)c2n[nH]cc2c1]

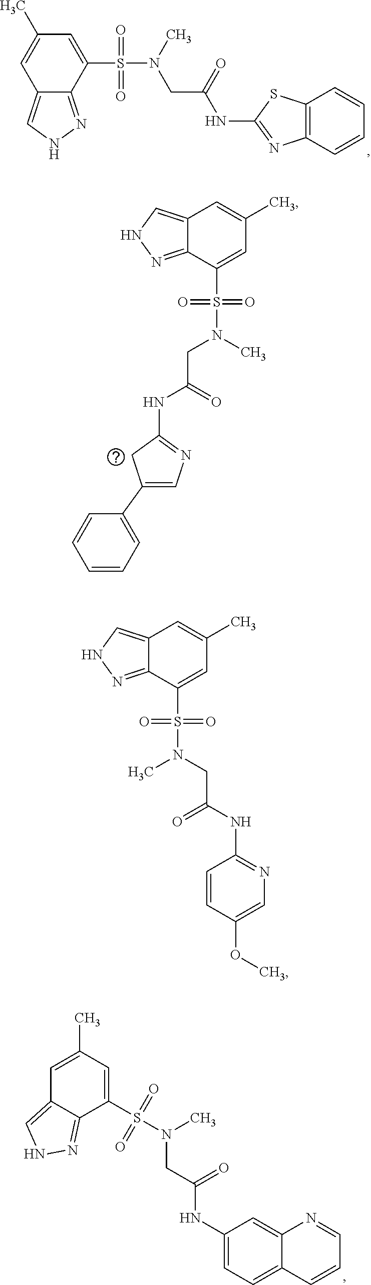 [SMILES: Cc1cc(S(=O)(=O)N(C)CC(=O)Nc2nc3ccccc3s2)c2n[nH]cc2c1 ; Cc1cc(S(=O)(=O)N(C)CC(=O)NC2=NC=C(c3ccccc3)C2)c2n[nH]cc2c1 ; COc1ccc(NC(=O)CN(C)S(=O)(=O)c2cc(C)cc3c[nH]nc23)nc1 ; Cc1cc(S(=O)(=O)N(C)CC(=O)Nc2ccc3cccnc3c2)c2n[nH]cc2c1]

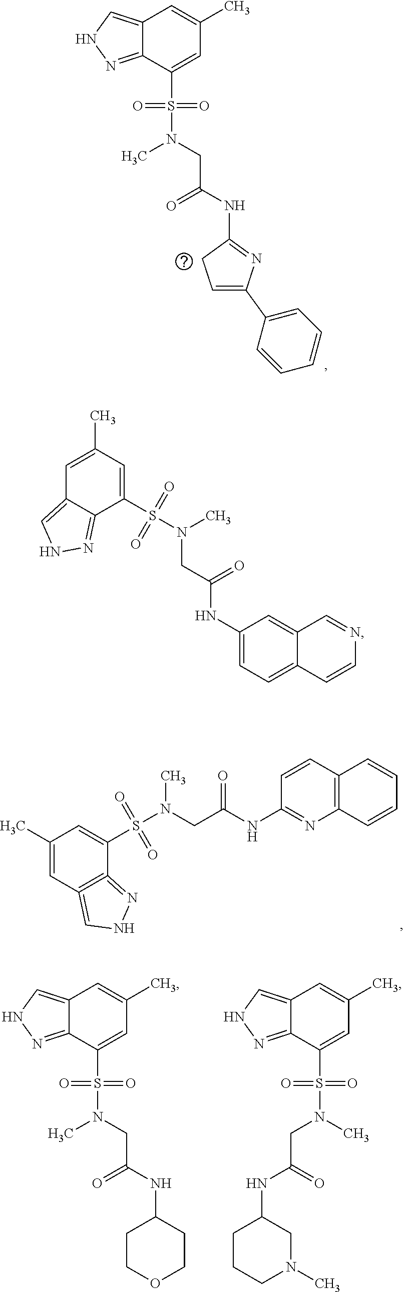

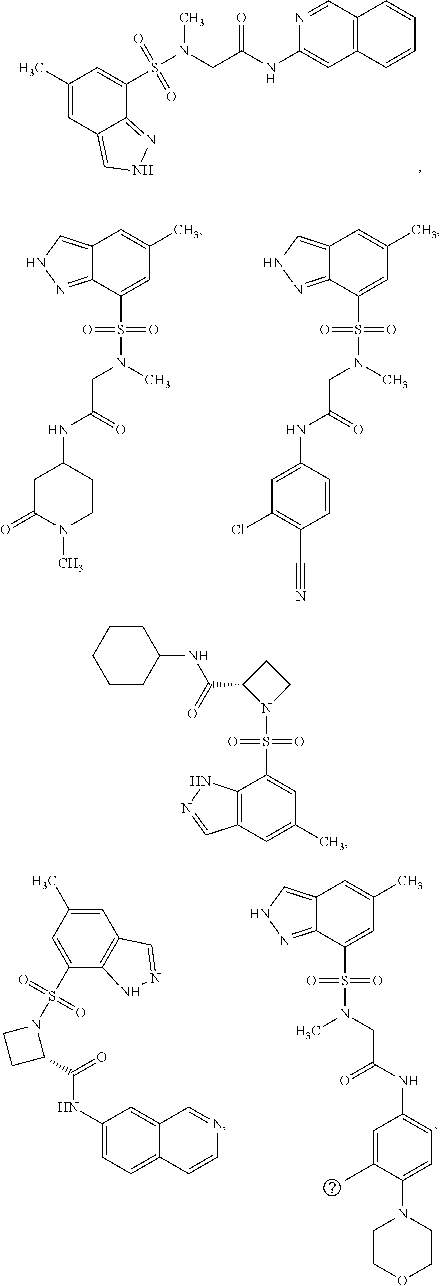

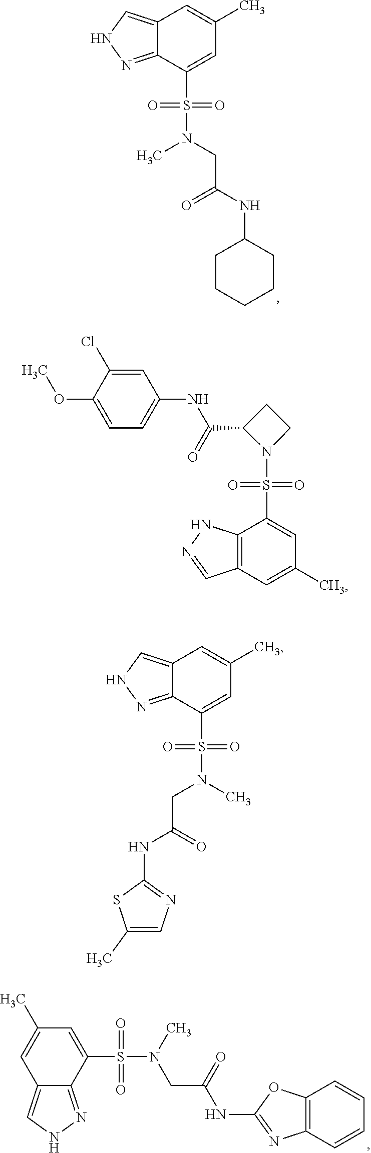 [SMILES: Cc1cc(S(=O)(=O)N(C)CC(=O)NC2CCCCC2)c2n[nH]cc2c1 ; COc1ccc(NC(=O)[C@@H]2CCN2S(=O)(=O)c2cc(C)cc3cn[nH]c23)cc1Cl ; Cc1cc(S(=O)(=O)N(C)CC(=O)Nc2ncc(C)s2)c2n[nH]cc2c1 ; Cc1cc(S(=O)(=O)N(C)CC(=O)Nc2nc3ccccc3o2)c2n[nH]cc2c1]

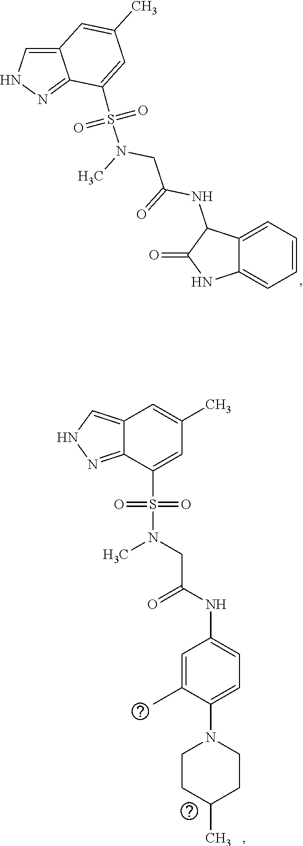 [SMILES: Cc1cc(S(=O)(=O)N(C)CC(=O)NC2C(=O)Nc3ccccc32)c2n[nH]cc2c1 ; Cc1cc(S(=O)(=O)N(C)CC(=O)Nc2ccc(N3CCC(C)CC3)c(C)c2)c2n[nH]cc2c1]

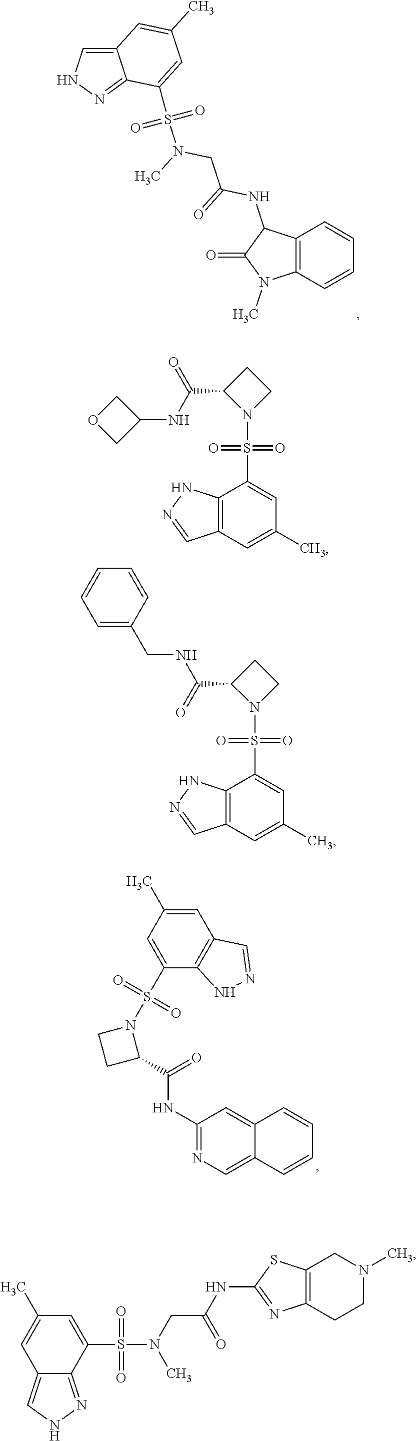 [SMILES: Cc1cc(S(=O)(=O)N(C)CC(=O)NC2C(=O)N(C)c3ccccc32)c2n[nH]cc2c1 ; Cc1cc(S(=O)(=O)N2CC[C@H]2C(=O)NC2COC2)c2[nH]ncc2c1 ; Cc1cc(S(=O)(=O)N2CC[C@H]2C(=O)NCc2ccccc2)c2[nH]ncc2c1 ; Cc1cc(S(=O)(=O)N2CC[C@H]2C(=O)Nc2cc3ccccc3cn2)c2[nH]ncc2c1 ; Cc1cc(S(=O)(=O)N(C)CC(=O)Nc2nc3c(s2)CN(C)CC3)c2n[nH]cc2c1]

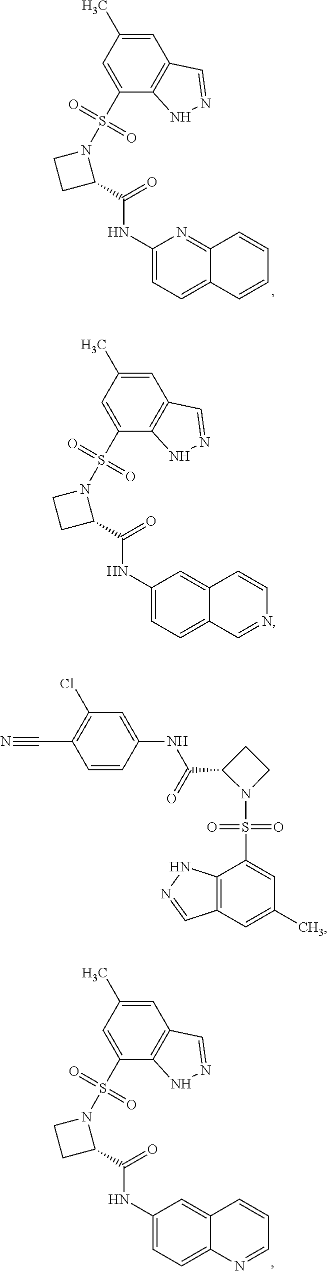 [SMILES: Cc1cc(S(=O)(=O)N2CC[C@H]2C(=O)Nc2ccc3ccccc3n2)c2[nH]ncc2c1 ; Cc1cc(S(=O)(=O)N2CC[C@H]2C(=O)Nc2ccc3cnccc3c2)c2[nH]ncc2c1 ; Cc1cc(S(=O)(=O)N2CC[C@H]2C(=O)Nc2ccc(C#N)c(Cl)c2)c2[nH]ncc2c1 ; Cc1cc(S(=O)(=O)N2CC[C@H]2C(=O)Nc2ccc3ncccc3c2)c2[nH]ncc2c1]

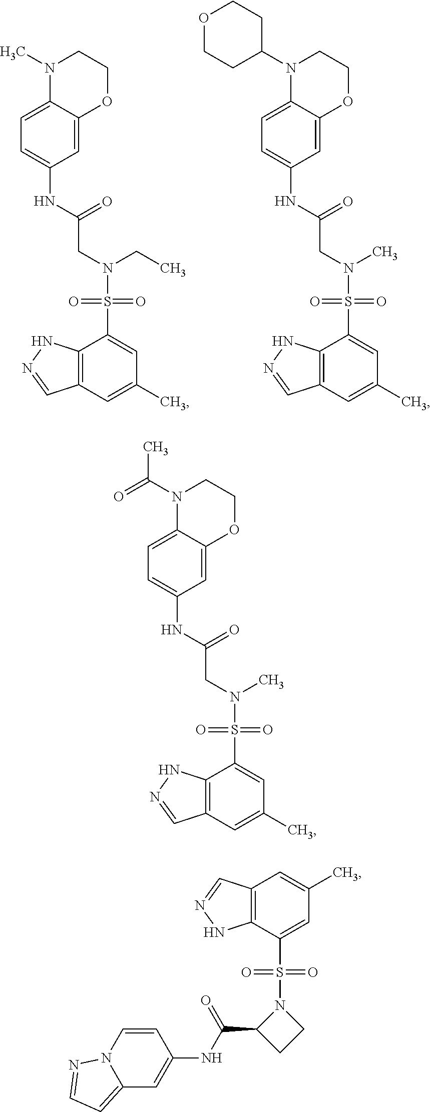

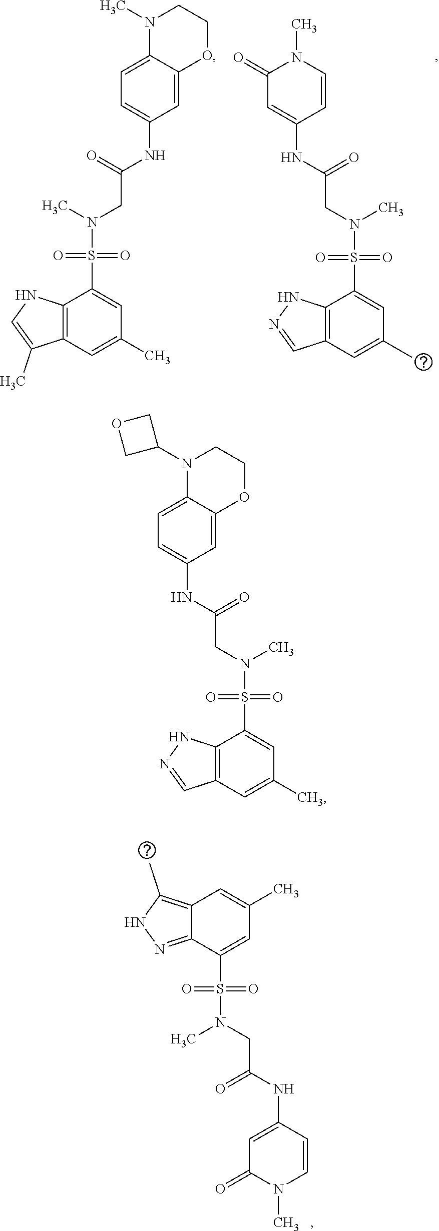

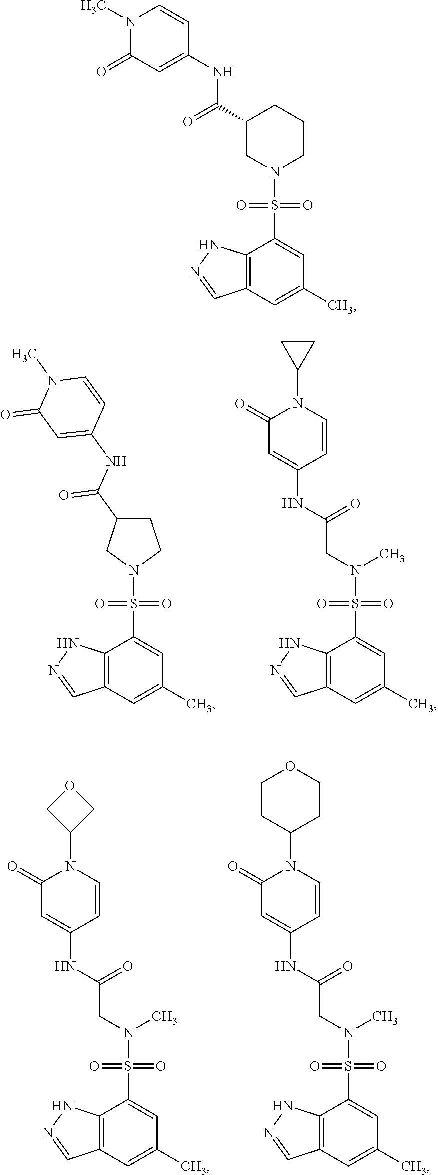

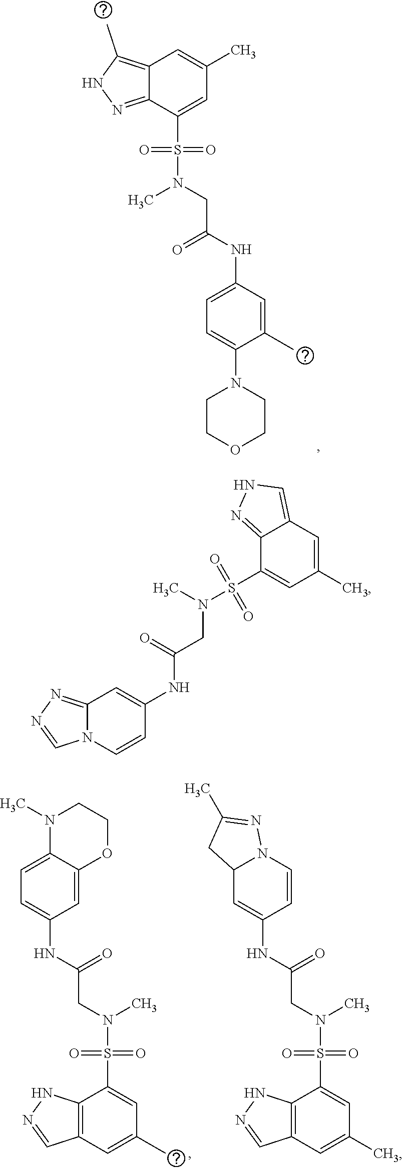

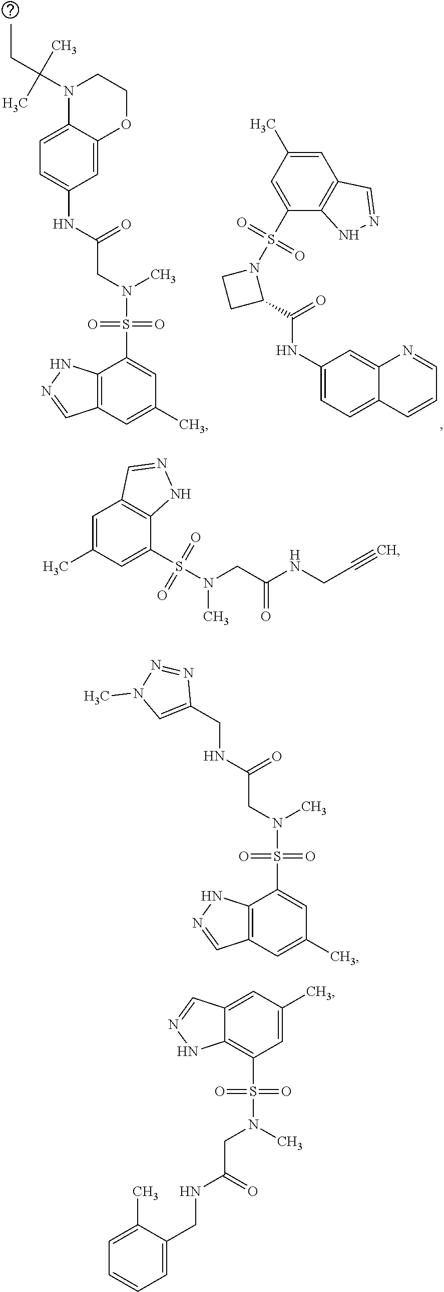

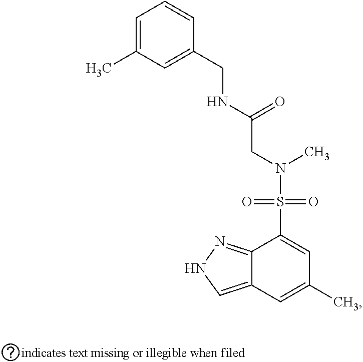 [SMILES: Cc1cccc(CNC(=O)CN(C)S(=O)(=O)c2cc(C)cc3c[nH]nc23)c1]

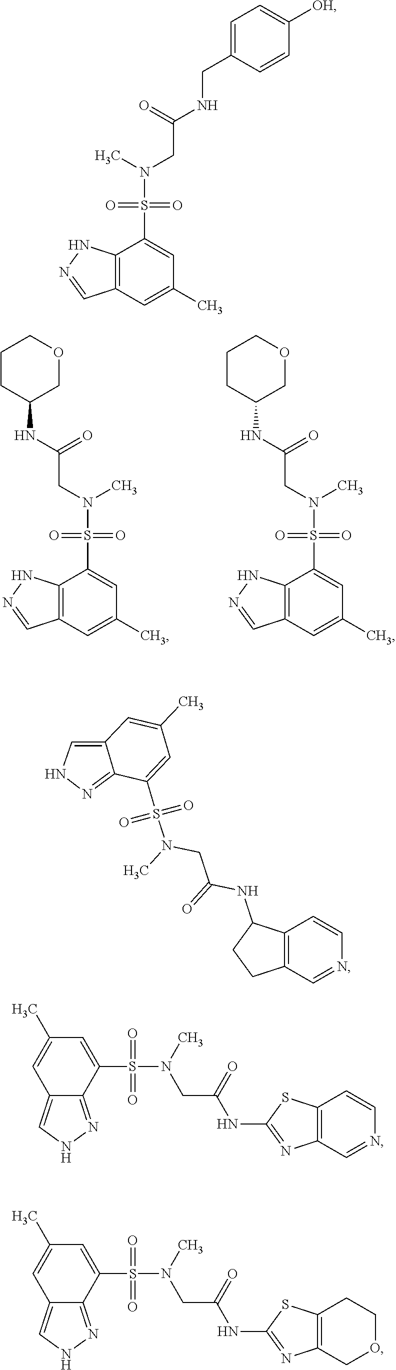 [SMILES: Cc1cc(S(=O)(=O)N(C)CC(=O)NCc2ccc(O)cc2)c2[nH]ncc2c1 ; Cc1cc(S(=O)(=O)N(C)CC(=O)N[C@H]2CCCOC2)c2[nH]ncc2c1 ; Cc1cc(S(=O)(=O)N(C)CC(=O)N[C@@H]2CCCOC2)c2[nH]ncc2c1 ; Cc1cc(S(=O)(=O)N(C)CC(=O)NC2CCc3cnccc32)c2n[nH]cc2c1 ; Cc1cc(S(=O)(=O)N(C)CC(=O)Nc2nc3cnccc3s2)c2n[nH]cc2c1 ; Cc1cc(S(=O)(=O)N(C)CC(=O)Nc2nc3c(s2)CCOC3)c2n[nH]cc2c1]

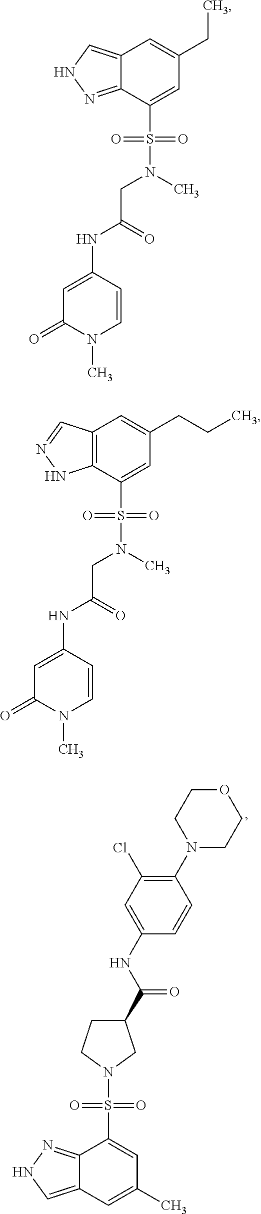 [SMILES: CCc1cc(S(=O)(=O)N(C)CC(=O)Nc2ccn(C)c(=O)c2)c2n[nH]cc2c1 ; CCCc1cc(S(=O)(=O)N(C)CC(=O)Nc2ccn(C)c(=O)c2)c2[nH]ncc2c1 ; Cc1cc(S(=O)(=O)N2CC[C@@H](C(=O)Nc3ccc(N4CCOCC4)c(Cl)c3)C2)c2n[nH]cc2c1]

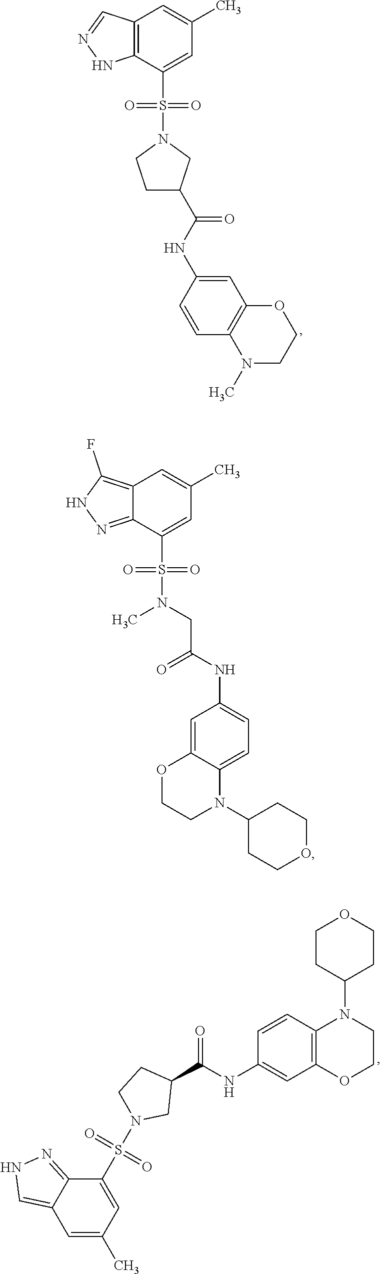 [SMILES: Cc1cc(S(=O)(=O)N2CCC(C(=O)Nc3ccc4c(c3)OCCN4C)C2)c2[nH]ncc2c1 ; Cc1cc(S(=O)(=O)N(C)CC(=O)Nc2ccc3c(c2)OCCN3C2CCOCC2)c2n[nH]c(F)c2c1 ; Cc1cc(S(=O)(=O)N2CC[C@@H](C(=O)Nc3ccc4c(c3)OCCN4C3CCOCC3)C2)c2n[nH]cc2c1]

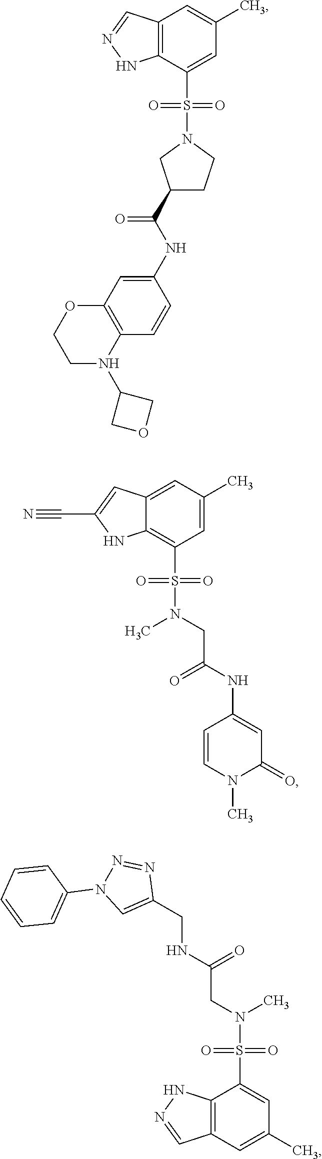 [SMILES: Cc1cc(S(=O)(=O)N2CC[C@@H](C(=O)Nc3ccc4c(c3)OCCN4C3COC3)C2)c2[nH]ncc2c1 ; Cc1cc(S(=O)(=O)N(C)CC(=O)Nc2ccn(C)c(=O)c2)c2[nH]c(C#N)cc2c1 ; Cc1cc(S(=O)(=O)N(C)CC(=O)NCc2cn(-c3ccccc3)nn2)c2[nH]ncc2c1]

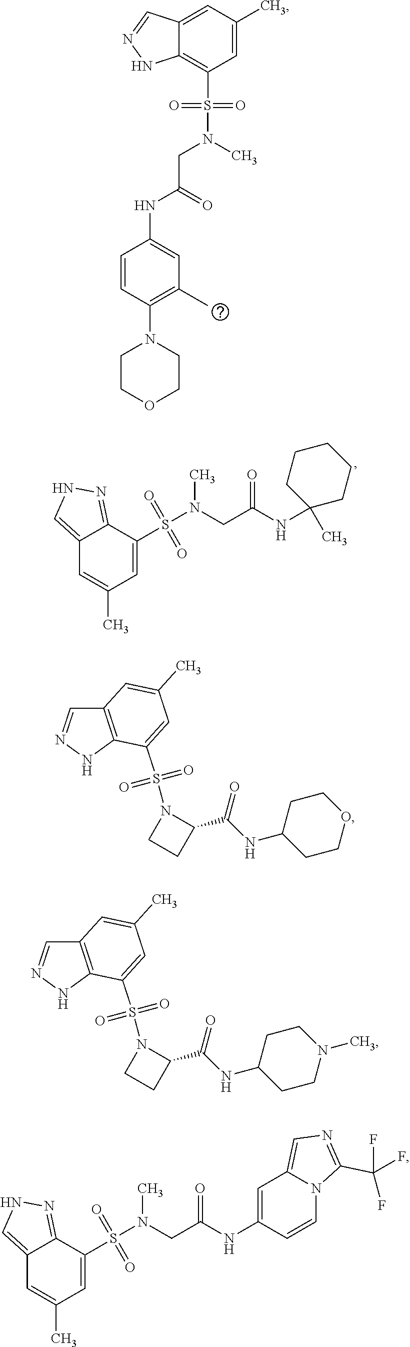 [SMILES: Cc1cc(S(=O)(=O)N(C)CC(=O)Nc2ccc(N3CCOCC3)c(C)c2)c2[nH]ncc2c1 ; Cc1cc(S(=O)(=O)N(C)CC(=O)NC2(C)CCCCC2)c2n[nH]cc2c1 ; Cc1cc(S(=O)(=O)N2CC[C@H]2C(=O)NC2CCOCC2)c2[nH]ncc2c1 ; Cc1cc(S(=O)(=O)N2CC[C@H]2C(=O)NC2CCN(C)CC2)c2[nH]ncc2c1 ; Cc1cc(S(=O)(=O)N(C)CC(=O)Nc2ccn3c(C(F)(F)F)ncc3c2)c2n[nH]cc2c1]

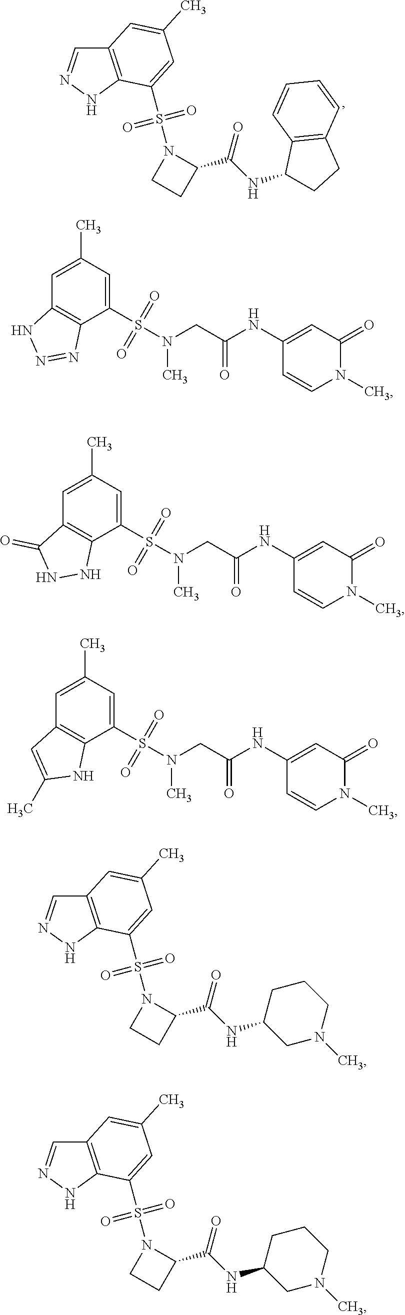 [SMILES: Cc1cc(S(=O)(=O)N2CC[C@H]2C(=O)N[C@H]2CCc3ccccc32)c2[nH]ncc2c1 ; Cc1cc(S(=O)(=O)N(C)CC(=O)Nc2ccn(C)c(=O)c2)c2nn[nH]c2c1 ; Cc1cc(S(=O)(=O)N(C)CC(=O)Nc2ccn(C)c(=O)c2)c2[nH][nH]c(=O)c2c1 ; Cc1cc(S(=O)(=O)N(C)CC(=O)Nc2ccn(C)c(=O)c2)c2[nH]c(C)cc2c1 ; Cc1cc(S(=O)(=O)N2CC[C@H]2C(=O)N[C@@H]2CCCN(C)C2)c2[nH]ncc2c1 ; Cc1cc(S(=O)(=O)N2CC[C@H]2C(=O)N[C@H]2CCCN(C)C2)c2[nH]ncc2c1]

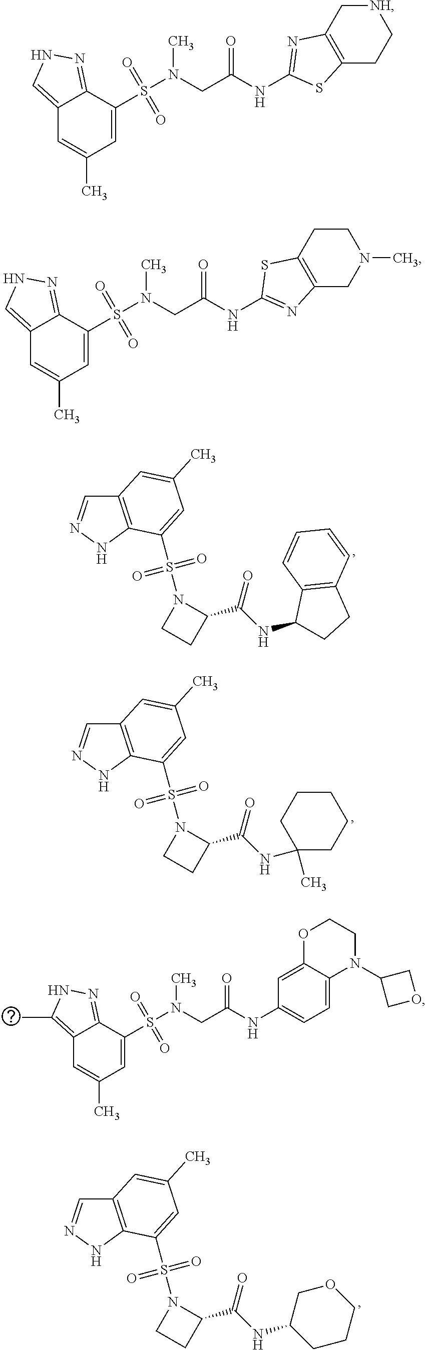

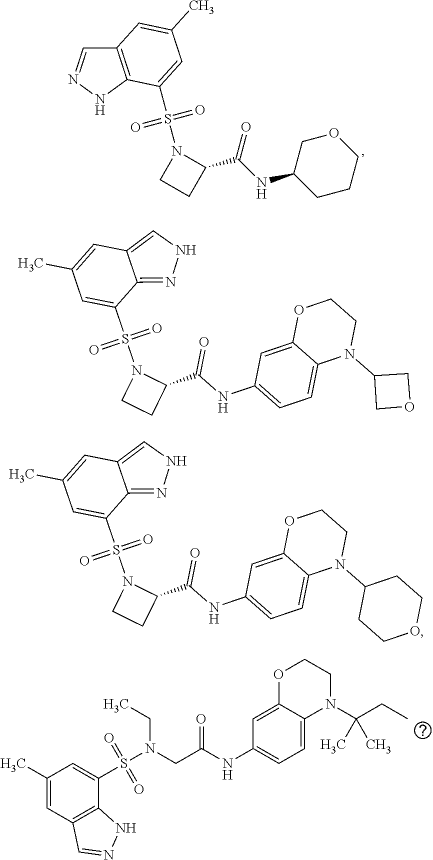

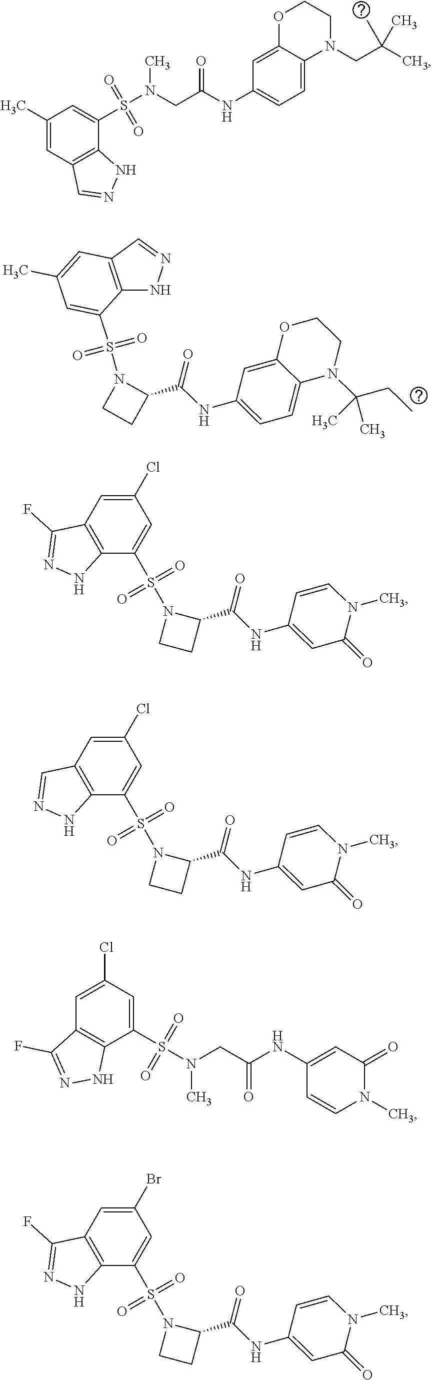 [SMILES: Cc1cc(S(=O)(=O)N(C)CC(=O)Nc2ccc3c(c2)OCCN3CC(C)(C)C)c2[nH]ncc2c1 ; CCC(C)(C)N1CCOc2cc(NC(=O)[C@@H]3CCN3S(=O)(=O)c3cc(C)cc4cn[nH]c34)ccc21 ; Cn1ccc(NC(=O)[C@@H]2CCN2S(=O)(=O)c2cc(Cl)cc3c(F)n[nH]c23)cc1=O ; Cn1ccc(NC(=O)[C@@H]2CCN2S(=O)(=O)c2cc(Cl)cc3cn[nH]c23)cc1=O ; CN(CC(=O)Nc1ccn(C)c(=O)c1)S(=O)(=O)c1cc(Cl)cc2c(F)n[nH]c12 ; Cn1ccc(NC(=O)[C@@H]2CCN2S(=O)(=O)c2cc(Br)cc3c(F)n[nH]c23)cc1=O]

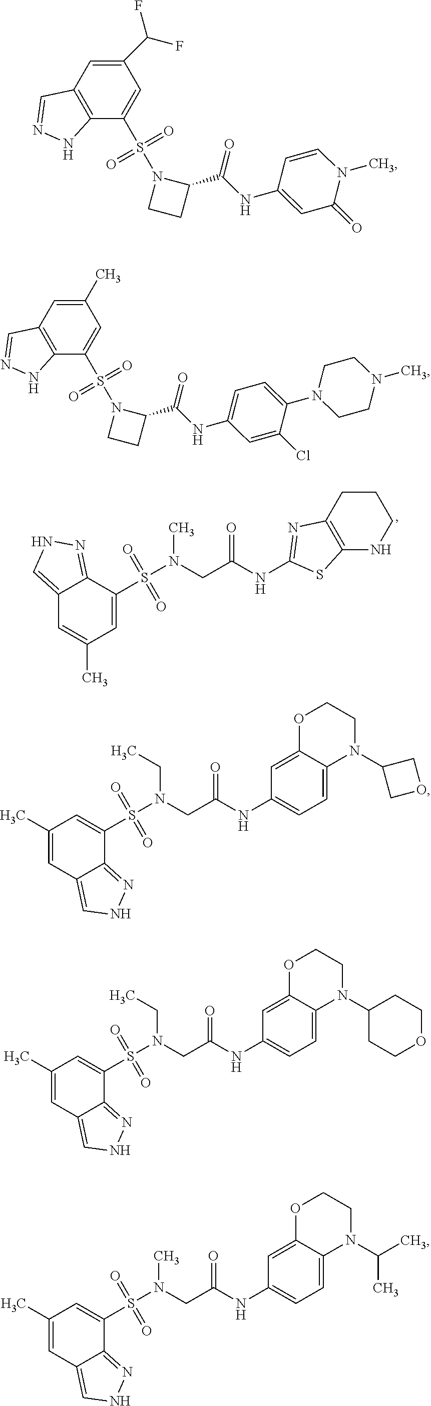 [SMILES: Cn1ccc(NC(=O)[C@@H]2CCN2S(=O)(=O)c2cc(C(F)F)cc3cn[nH]c23)cc1=O ; Cc1cc(S(=O)(=O)N2CC[C@H]2C(=O)Nc2ccc(N3CCN(C)CC3)c(Cl)c2)c2[nH]ncc2c1 ; Cc1cc(S(=O)(=O)N(C)CC(=O)Nc2nc3c(s2)NCCC3)c2n[nH]cc2c1 ; CCN(CC(=O)Nc1ccc2c(c1)OCCN2C1COC1)S(=O)(=O)c1cc(C)cc2c[nH]nc12 ; CCN(CC(=O)Nc1ccc2c(c1)OCCN2C1CCOCC1)S(=O)(=O)c1cc(C)cc2c[nH]nc12 ; Cc1cc(S(=O)(=O)N(C)CC(=O)Nc2ccc3c(c2)OCCN3C(C)C)c2n[nH]cc2c1]

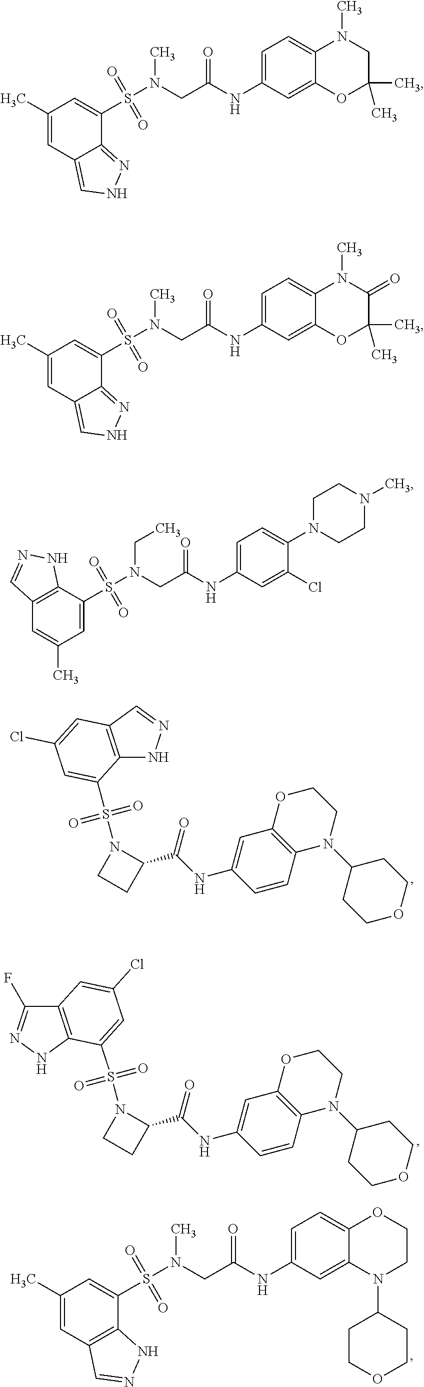

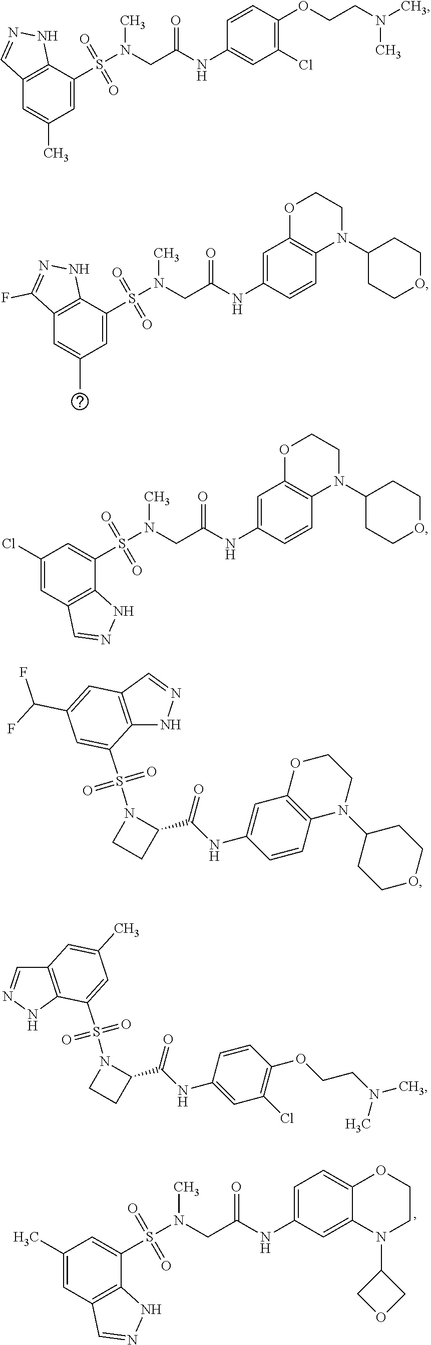 [SMILES: Cc1cc(S(=O)(=O)N(C)CC(=O)Nc2ccc(OCCN(C)C)c(Cl)c2)c2[nH]ncc2c1 ; Cc1cc(S(=O)(=O)N(C)CC(=O)Nc2ccc3c(c2)OCCN3C2CCOCC2)c2[nH]nc(F)c2c1 ; CN(CC(=O)Nc1ccc2c(c1)OCCN2C1CCOCC1)S(=O)(=O)c1cc(Cl)cc2cn[nH]c12 ; O=C(Nc1ccc2c(c1)OCCN2C1CCOCC1)[C@@H]1CCN1S(=O)(=O)c1cc(C(F)F)cc2cn[nH]c12 ; Cc1cc(S(=O)(=O)N2CC[C@H]2C(=O)Nc2ccc(OCCN(C)C)c(Cl)c2)c2[nH]ncc2c1 ; Cc1cc(S(=O)(=O)N(C)CC(=O)Nc2ccc3c(c2)N(C2COC2)CCO3)c2[nH]ncc2c1]

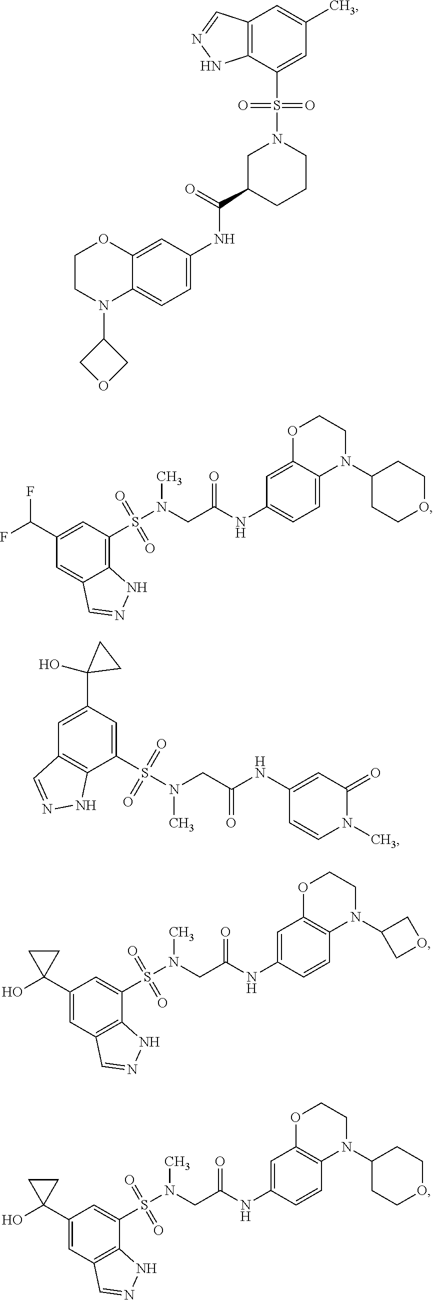

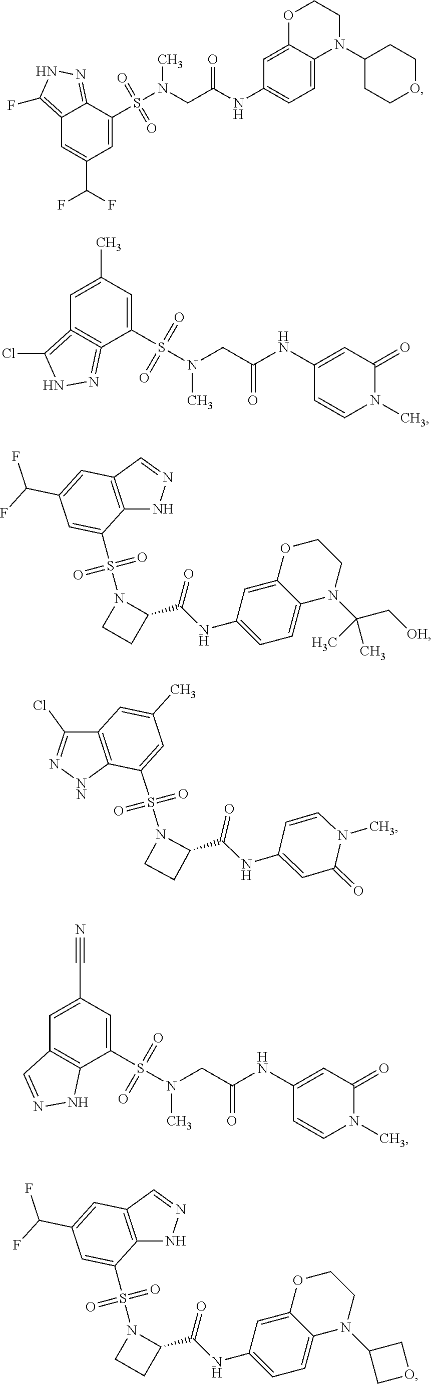

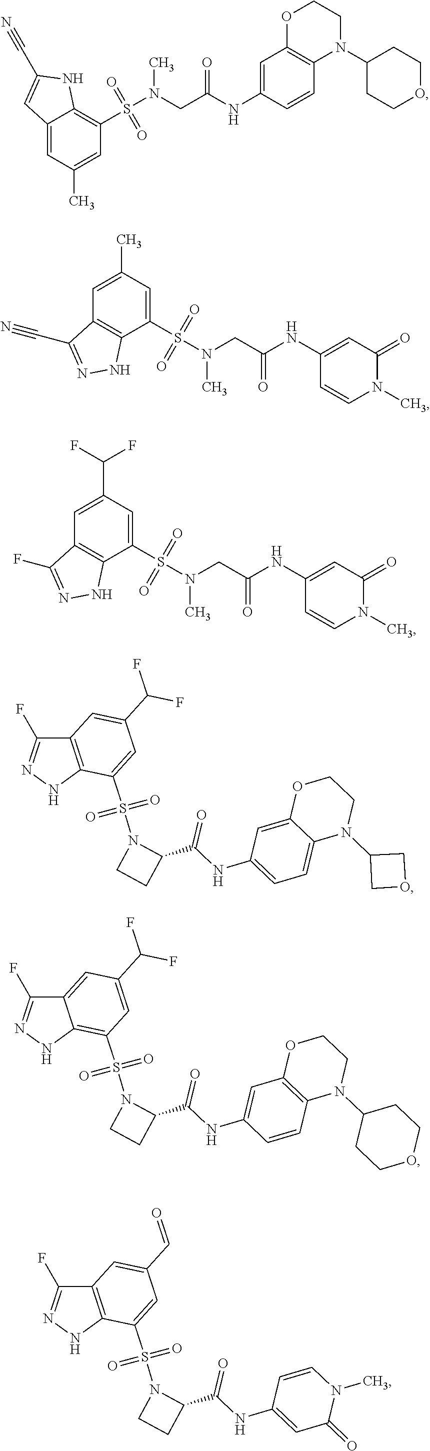

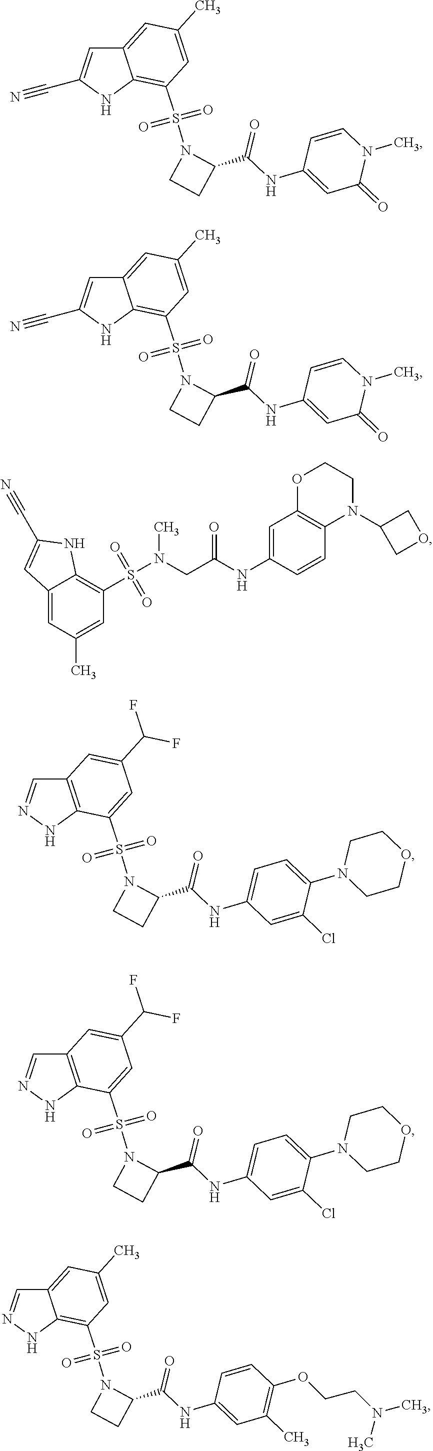

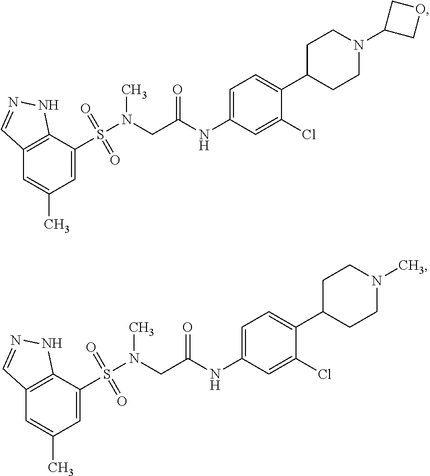 [SMILES: Cc1cc(S(=O)(=O)N(C)CC(=O)Nc2ccc(C3CCN(C4COC4)CC3)c(Cl)c2)c2[nH]ncc2c1 ; Cc1cc(S(=O)(=O)N(C)CC(=O)Nc2ccc(C3CCN(C)CC3)c(Cl)c2)c2[nH]ncc2c1]

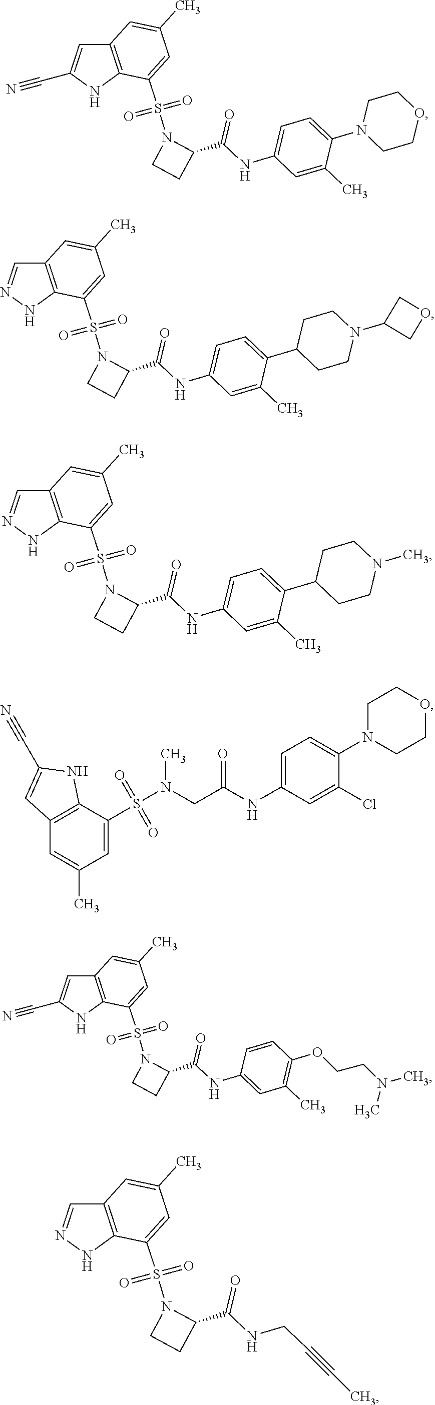 [SMILES: Cc1cc(S(=O)(=O)N2CC[C@H]2C(=O)Nc2ccc(N3CCOCC3)c(C)c2)c2[nH]c(C#N)cc2c1 ; Cc1cc(S(=O)(=O)N2CC[C@H]2C(=O)Nc2ccc(C3CCN(C4COC4)CC3)c(C)c2)c2[nH]ncc2c1 ; Cc1cc(S(=O)(=O)N2CC[C@H]2C(=O)Nc2ccc(C3CCN(C)CC3)c(C)c2)c2[nH]ncc2c1 ; Cc1cc(S(=O)(=O)N(C)CC(=O)Nc2ccc(N3CCOCC3)c(Cl)c2)c2[nH]c(C#N)cc2c1 ; Cc1cc(S(=O)(=O)N2CC[C@H]2C(=O)Nc2ccc(OCCN(C)C)c(C)c2)c2[nH]c(C#N)cc2c1 ; CC#CCNC(=O)[C@@H]1CCN1S(=O)(=O)c1cc(C)cc2cn[nH]c12]

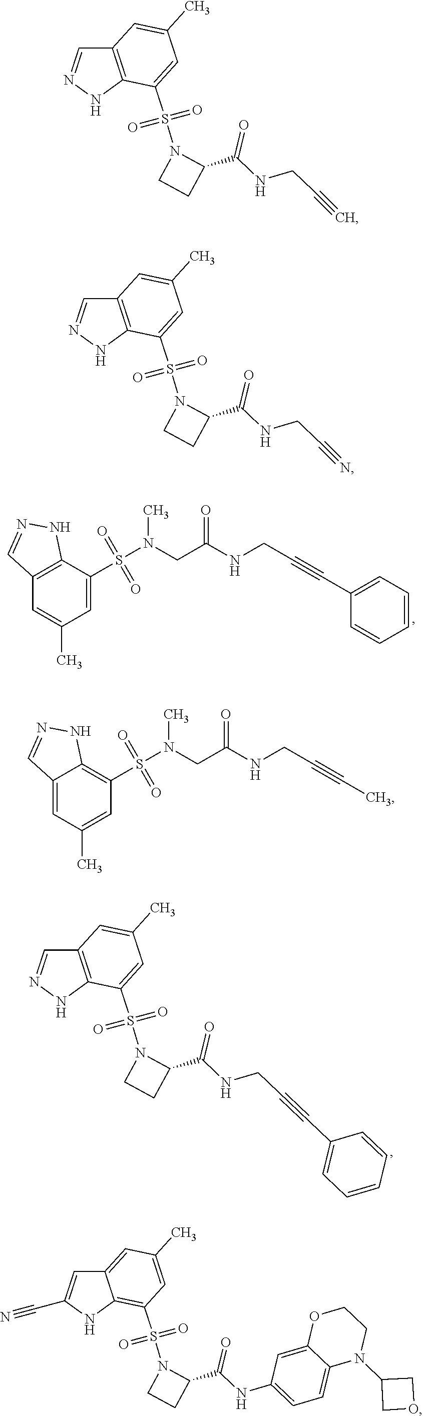 [SMILES: C#CCNC(=O)[C@@H]1CCN1S(=O)(=O)c1cc(C)cc2cn[nH]c12 ; Cc1cc(S(=O)(=O)N2CC[C@H]2C(=O)NCC#N)c2[nH]ncc2c1 ; Cc1cc(S(=O)(=O)N(C)CC(=O)NCC#Cc2ccccc2)c2[nH]ncc2c1 ; CC#CCNC(=O)CN(C)S(=O)(=O)c1cc(C)cc2cn[nH]c12 ; Cc1cc(S(=O)(=O)N2CC[C@H]2C(=O)NCC#Cc2ccccc2)c2[nH]ncc2c1 ; Cc1cc(S(=O)(=O)N2CC[C@H]2C(=O)Nc2ccc3c(c2)OCCN3C2COC2)c2[nH]c(C#N)cc2c1]

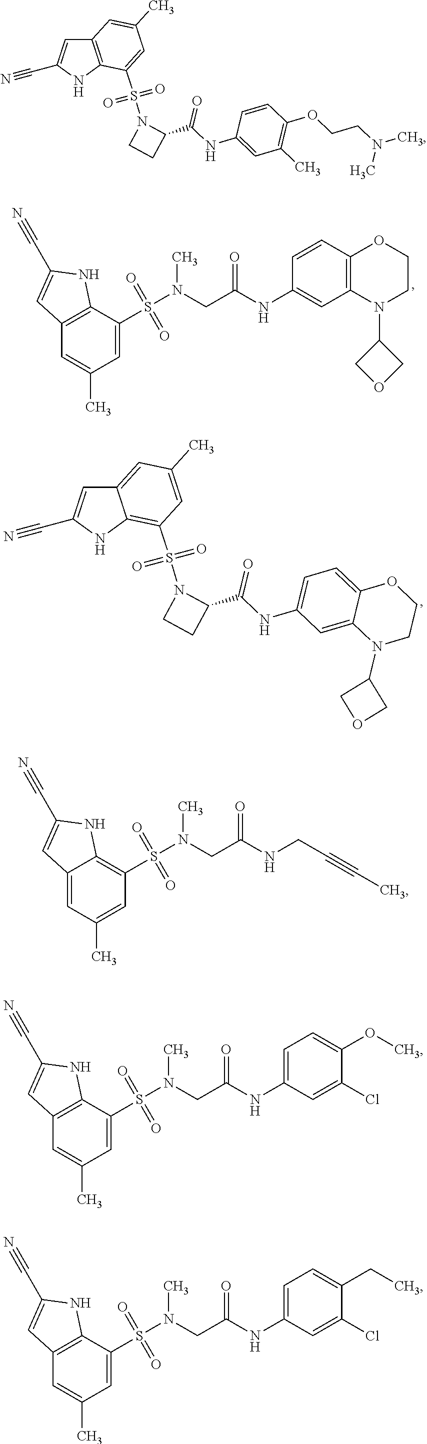 [SMILES: Cc1cc(S(=O)(=O)N2CC[C@H]2C(=O)Nc2ccc(OCCN(C)C)c(C)c2)c2[nH]c(C#N)cc2c1 ; Cc1cc(S(=O)(=O)N(C)CC(=O)Nc2ccc3c(c2)N(C2COC2)CCO3)c2[nH]c(C#N)cc2c1 ; Cc1cc(S(=O)(=O)N2CC[C@H]2C(=O)Nc2ccc3c(c2)N(C2COC2)CCO3)c2[nH]c(C#N)cc2c1 ; CC#CCNC(=O)CN(C)S(=O)(=O)c1cc(C)cc2cc(C#N)[nH]c12 ; COc1ccc(NC(=O)CN(C)S(=O)(=O)c2cc(C)cc3cc(C#N)[nH]c23)cc1Cl ; CCc1ccc(NC(=O)CN(C)S(=O)(=O)c2cc(C)cc3cc(C#N)[nH]c23)cc1Cl]

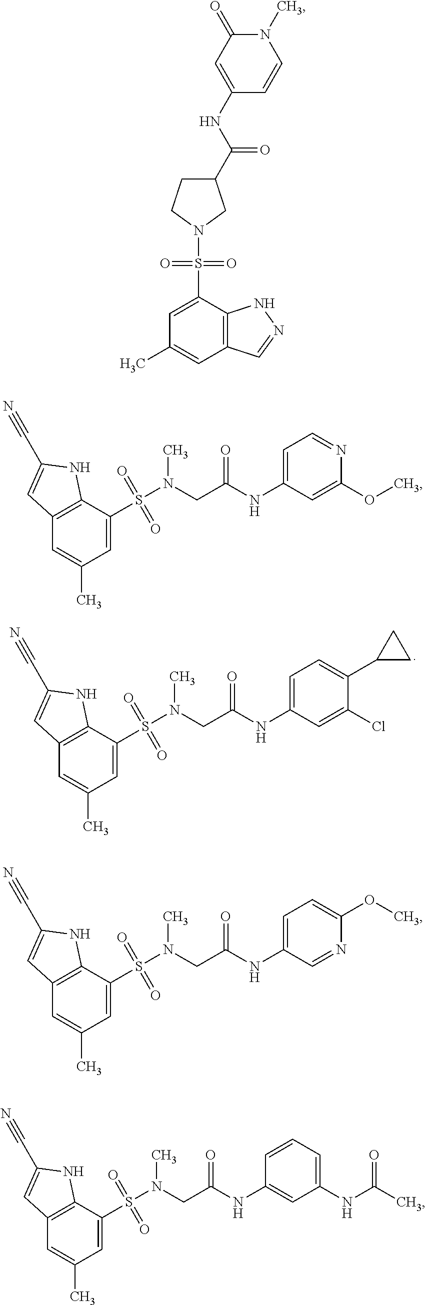 [SMILES: Cc1cc(S(=O)(=O)N2CCC(C(=O)Nc3ccn(C)c(=O)c3)C2)c2[nH]ncc2c1 ; COc1cc(NC(=O)CN(C)S(=O)(=O)c2cc(C)cc3cc(C#N)[nH]c23)ccn1 ; Cc1cc(S(=O)(=O)N(C)CC(=O)Nc2ccc(C3CC3)c(Cl)c2)c2[nH]c(C#N)cc2c1 ; COc1ccc(NC(=O)CN(C)S(=O)(=O)c2cc(C)cc3cc(C#N)[nH]c23)cn1 ; CC(=O)Nc1cccc(NC(=O)CN(C)S(=O)(=O)c2cc(C)cc3cc(C#N)[nH]c23)c1]

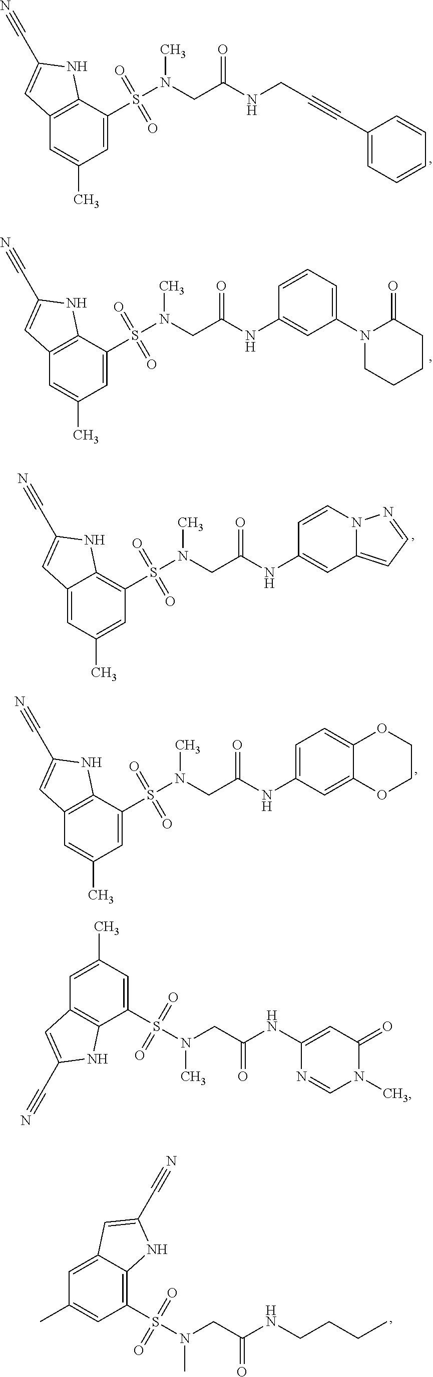

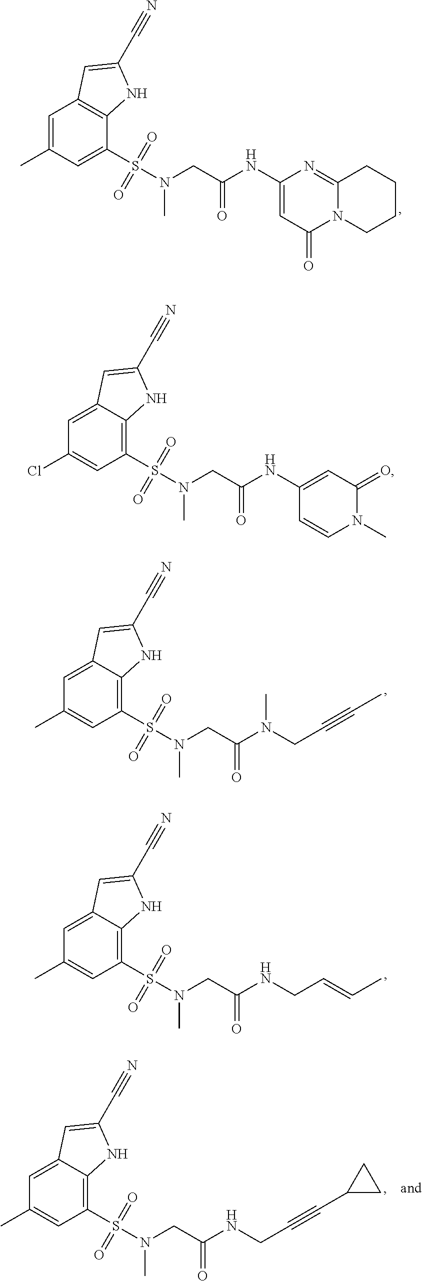

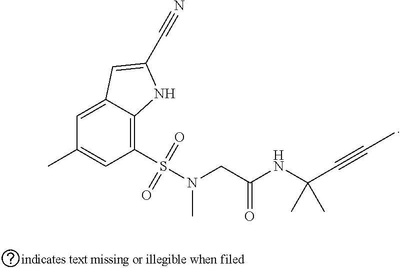 [SMILES: CC#CC(C)(C)NC(=O)CN(C)S(=O)(=O)c1cc(C)cc2cc(C#N)[nH]c12]

In certain aspects, the present disclosure provides pharmaceutical compositions, comprising a compound disclosed herein and a pharmaceutically acceptable carrier.

In certain aspects, the present disclosure provides methods of inhibiting or killing Mycobacterium tuberculosis in vitro, comprising contacting Mycobacterium tuberculosis with a compound disclosed herein.

In certain aspects, the present disclosure provides methods of treating tuberculosis, comprising administering to a subject in need thereof a compound disclosed herein. In certain embodiments, the subject is a mammal. In certain embodiments, the subject is a human.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

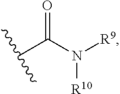

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

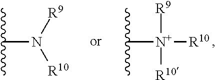

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes optionally substituted (i.e., substituted or unsubstituted) single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

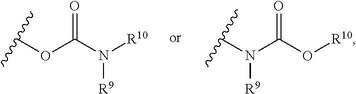

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted (i.e., are optionally substituted) at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” includes optionally substituted (i.e., substituted or unsubstituted) aromatic single ring structures, preferably 5- to 7-membered rings, more preferably 5- to 6-membered rings, whose ring structures include at least one heteroatom, preferably one to four heteroatoms, more preferably one or two heteroatoms.

The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to optionally substituted (i.e., substituted or unsubstituted) non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be optionally substituted (i.e., are substituted or unsubstituted). In certain embodiments, each ring of the polycycle contains from 3 to 10 atoms in the ring, preferably from 5 to 7.

The term “sulfate” is art-recognized and refers to the group —OSO3H, or a pharmaceutically acceptable salt thereof.

The term “sulfonamide” is art-recognized and refers to the group represented by the general formulae

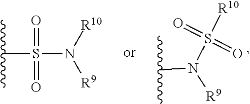

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

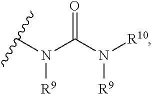

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

As used herein the term “healthy weight” refers to an individual with a body mass index between 18.5 and 24.9.

### EXAMPLES

The invention now being generally described, it will be more readily understood by reference to the following examples which are included merely for purposes of illustration of certain aspects and embodiments of the present invention, and are not intended to limit the invention.

### Exemplary 1. Discovery of Indazole Sulfonamides as LPD Inhibitors

### Materials and Methods

All chemicals were from Sigma. Recombinant protein production and purification (Mtb Lpd WT, Mtb Lpd R93A) was performed as reported previously. Purified recombinant human Lpd was a generous gift from Prof. M. Patel, University at Buffalo, SUNY.

LPD Assay. Lpd activity was measured by the DTNB assay according to previously published procedures. For preincubation of Lpd with the inhibitor, Lpd was dispensed into the wells, compounds added at specified concentrations, mixed and preincubated at RT for 30 min without any other components. Reaction was started by adding assay mix containing the substrates (lipoamide, NADH) and DTNB and the TNB production was recorded over time at RT in the SpectraMax plate reader at 412 nm. For time-dependent measurements compounds were added to wells containing assay mix and the reaction was started by the addition of Lpd protein and TNB production was followed over time in SpectraMax plate reader at 412 nm. Final concentrations of components in the reaction mixture were as following: 150 μM NADH, 150 μM DTNB, 40 μM NAD⁺, 75 μM lipoamide in 25 mM potassium phosphate, 1 mM ETDA pH 7.0. Lpd protein was tested at variable concentrations specified in the graphs. Protein concentration was determined by Bradford, Lpd concentration is provided for monomeric enzyme; active species is a dimer and binds 2 molecules of compound per dimer. Progress curves were fitted to first order association in GraphPad Prism to calculate k_{obs}. Kiₐₚₚ for tight binding inhibitors were calculated by fitting the fractional velocity data to the Morrison Ki equation in GraphPad Prism.

MIC assay. MIC values were determined on WT Mycobacterium tuberculosis H37Rv strain in 96 well plates in 200 μL of Middlebrook 7H9 medium pH 6.6 with 0.2% glycerol, 0.02% tyloxapol and 10% ADN (5% fatty-acid free BSA (Roche), 2% dextrose, 0.85% NaCl). Starting bacterial inoculum was 0.01 (OD₅₈₀). Inhibitors were tested at 2-fold serial dilutions from 100 μM to 0.1 μM. MICs were defined as compound concentrations that inhibited bacterial growth >90% after 10 days of incubation at 37 C in 5% CO₂, 95% humidified air.

BMDM. BMDM were differentiated as reported for 6-7 days in complete DMEM (4.5 g/l glucose, 10% FBS, 1% HEPES, 1% sodium pyruvate, 1% L-glutamine) supplemented with 20% L929 cell-conditioned medium (LCM). Cells were collected in 0.5 mM EDTA in PBS, washed with 10% LCM in complete DMEM, counted by hemocytometer and plated in 48-well plates (2.0-2.5×10⁵ cells/well) in 0.5 mL 10% LCM in complete DMEM. No antibiotics were used at any stage in any experiments, except for rifampin where indicated. All cultures were incubated at 37 C in 5% CO₂, 95% humidified air.

Infection of BMDM with Mtb. BMDM were plated in 48-well plates (2.0-2.5×10⁵ cells/well) in 0.5 mL 10% LCM in complete DMEM. Where indicated, mouse IFNgama (final 10 ng/mL, Roche) was added and cells were left to adhere overnight. A single-cell suspension of Mtb H37Rv in PBS with 0.02% tyloxapol was added in 50 μL to achieve MOI of 0.1. Four h later, the medium was removed, the monolayers washed twice with warm PBS and 0.5 mL fresh 10% LCM in complete DMEM replaced. BMDM were observed daily and photomicrographs recorded. Only wells with intact BMDM monolayers were used for determination of CFU. Test agents were added 24 h after Mtb infection by replacing the medium in wells with BMDM with 10% LCM in complete DMEM containing the indicated final concentrations of inhibitors. Data are presented according to the time after treatment as opposed to the time after infection. At each time point, the medium was removed, monolayers washed with warm PBS and BMDM lysed with 100 μL of 0.5% Triton X100 in PBS. PBS (400 μL) was immediately added to wells to dilute the Triton X100 to 0.10% to minimize any impact on Mtb viability. Samples were serially diluted and 10 μL plated on 7H11 agar plates incubated at 37 C for CFU enumeration 3 weeks later.

ITC measurements. All measurements were performed on MicroCal Auto-ITC200 instrument. Lpd was used at 10 or 20 μM and compound C at 250 μM. Measurements were performed in 25 mM potassium phosphate buffer pH 7.0, 2.5% DMSO −/+100 μM NADH or in 20 mM triethanolamine buffer pH 7.8, 2.5% DMSO−/+100 μM NADH. Data analysis was performed on the Affinimeter software using the Stoichiometric Equilibria model assuming Free Species M₁A₁. K_{d} was calculated as a reciprocal of Kₐ; free energy was calculated by the formula ΔG=R*T*lnK_{d}, where R=1.9858775 cal*M⁻¹*K⁻¹ and T=298.15 K; TΔS was calculated from the formula ΔG=ΔH−TΔS.

SPR. All measurements were performed on the eight needle high-sensitivity SPR Biacore 8K system (Cytiva). CM5 sensor chip was used for all experiments, Mtb and human WT proteins were immobilized on the matrix via amine coupling at pH 5.0 to achieve comparable RU (11342±58.96 for Mtb and 12735±125.49 for human Lpd). Parallel kinetics was run in 25 mM potassium phosphate, pH 7.0, 0.05% Surfactant P20, 1% DMSO−/+100 μM NADH with 4 variable concentrations of compound C; contact time was 120 sec, dissociation time 300 sec, flow rate 50 μl/min. Data evaluation and fitting was performed with Biacore Insight Evaluation Software.

### Results and Discussion

Profiling of compound A demonstrated an improvement in enzyme inhibition, as well as whole cell activity with a MIC₉₀ of 25 μM when tested against WT Mtb grown in a carbohydrate-based medium. In vitro ADME-tox profiling of compound A showed that while aqueous solubility was maintained, the compound suffered from rapid microsomal clearance. Following the SAR generated for the aminopyridines, compounds B and C were synthesized. Compound A demonstrated an improvement in MIC compared to compound A but did not maintain the improved microsomal stability associated with the morpholine substitution. However, N-methyl pyridone substituted compound A maintained the improved properties and further increased the MIC potency to 3.1 μM.

TABLE 1
Antibacterial Activity, and ADME Profiling of Sulfonamide Analogs

| 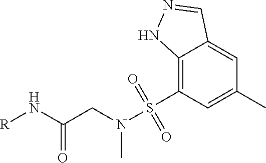 | | | | | | |
|---|---|---|---|---|---|---|
| | | | | | | a |
| 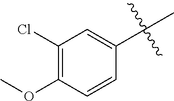 [SMILES: *c1ccc(OC)c(Cl)c1] | | | | | | |
| | | | | | | b |
| 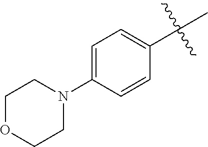 [SMILES: *c1ccc(N2CCOCC2)cc1] | | | | | | |
| | | | | | | c |
| 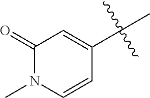 [SMILES: *c1ccn(C)c(=O)c1] | | | | | | |
| | | LPD IC₅₀ | Mtb MIC₉₀ | | | |
| Cmpd. # | R | (μM) | (μM) | Sol | hLM | LogD |
| A | a | 0.035 | 25 | 18 | >768 | 2.26 |
| B | b | 0.029 | 12 | 23 | 195 | 2.55 |
| C | c | 0.045 | 3.1 | >110 | −11 | 0.83 |

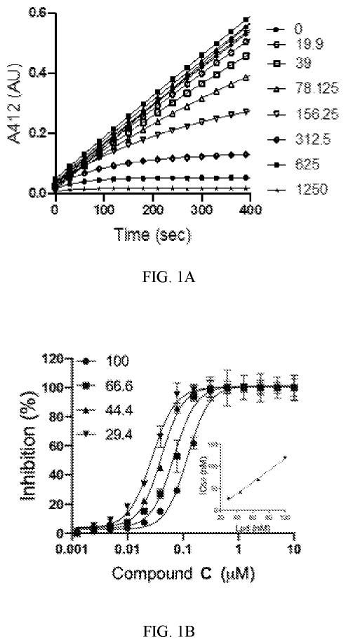

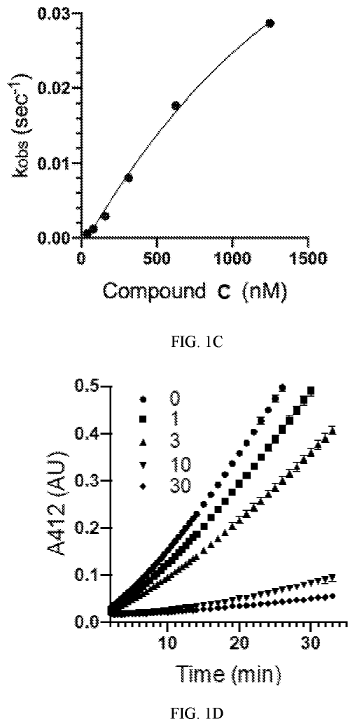

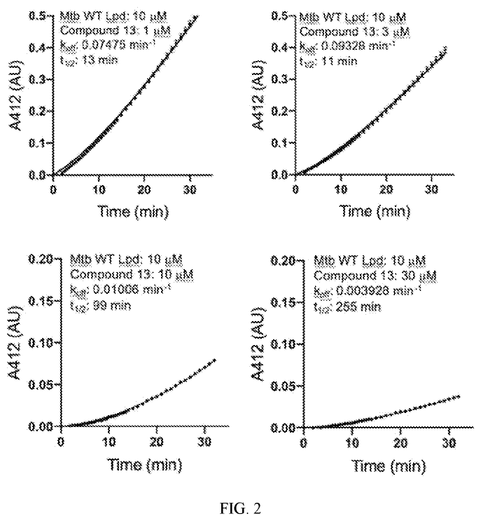

Compound C exhibited time-dependent inhibition of purified recombinant Mtb Lpd following reaction initiation by the addition of enzyme (FIG. 1A). Upon 30 min preincubation, IC₅₀ values tracked linearly with variable enzyme concentration, nearing the values for the concentrations of Lpd and suggesting a slow onset, tight binding interaction (FIG. 1B). Fitting the progress curve data to the first order rate kinetics produced values of k_{obs} that increased with a rising molar excess of inhibitor to Lpd (FIG. 1C). The relationship was not strictly linear, suggesting an induced fit model for compound C association with Lpd. Ki^{app} (9±6.9 nM) was calculated from fitting the inhibition data expressed as fractional velocity at a given inhibitor concentration to the Morrison equation for tight binding inhibitors. Preincubation of Lpd with compound C with subsequent 500-fold dilution into the assay mix led to rapid recovery of activity at subequimolar concentrations of inhibitor during preincubation and fractional recovery with a long lag period at a molar excess of compound C (FIG. 1D). Fitting the recovery experimental data for compound C to the integrated rate equation to determine the dissociation rate constant produced k_{off} value of 0.084 min⁻¹, which corresponded to a 12 min half-life (t_{1/2}) of the Lpd-C complex (FIG. 2). At equimolar and higher concentrations of compound C the recovery slowed down, with over 10-fold reduction in k_{off} values corresponding to hours-long t_{1/2} (FIG. 2). This sustained inhibition at higher concentrations likely reflects continuous re-binding of the dissociated (and/or excess) inhibitor. Nevertheless, enzymatic data suggested extended residence time of compound C on Mtb's Lpd under conditions when compound C is at a molar excess to Lpd. Thus, achieving intra-Mtb levels of compound C in excess of Lpd intracellular concentration should result in sustained target engagement and drive the inhibition of Lpd activity in vivo.

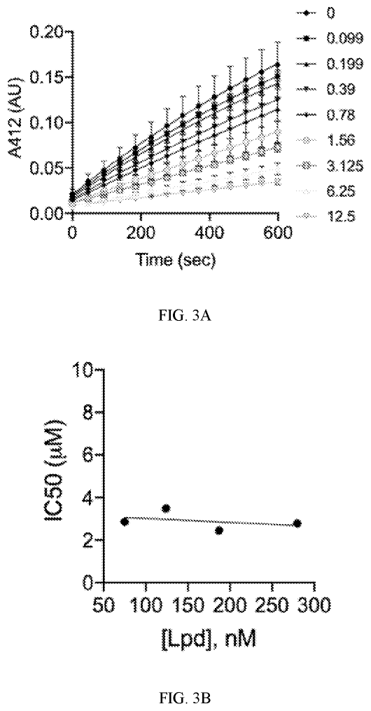

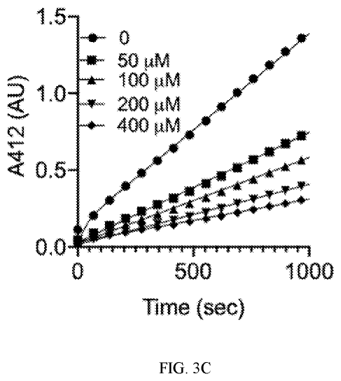

In contrast, when compound C was tested against the recombinant human Lpd in a similar manner, no time-dependent inhibition was observed (FIG. 3A), IC₅₀ values did not change significantly with variable enzyme concentration (FIG. 3B), and inhibition was rapidly reversible upon dilution, even at high molar excess of inhibitor (FIG. 3C). Taken together, these results demonstrate that compound C is a significantly less potent (Kᵢ=1.13±0.18 μM), rapid equilibrium inhibitor of human Lpd.

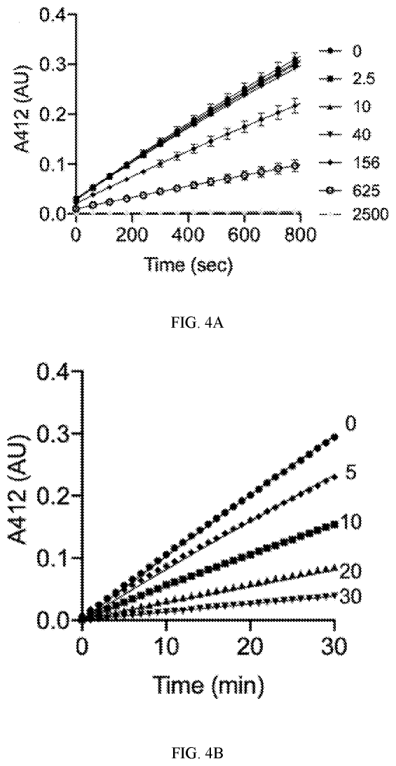

The crystal structure of Mtb Lpd suggested a critical role for Arg93 in maintaining access to the lipoamide binding site as it was able to adopt two different conformations. Human Lpd has a Leu residue at that position and the lack of coordination through Arg93 may have contributed to lower affinity of compound C for the human homolog. An Mtb Lpd mutant enzyme with the single amino acid substitution R93A was tested to determine if Arg93 contact contributes to binding affinity and affects the dissociation rate of compound C. No time-dependent inhibition of Mtb Lpd R93A activity in the presence of compound C was observed, progress curves with increasing compound C were linear from the onset (FIG. 4A), and Kᵢ was calculated to be 218 nM. Lpd R93A preincubation with compound C and dilution into the assay mix to recover activity resulted in linear progress curves without any lag, suggesting rapid dissociation of compound C from Mtb Lpd R93A (FIG. 4B).

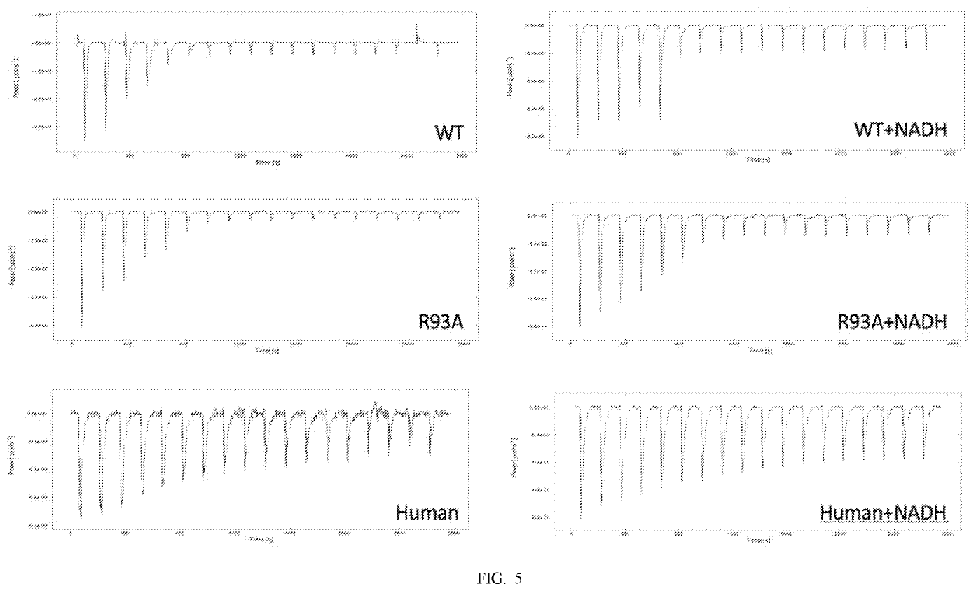

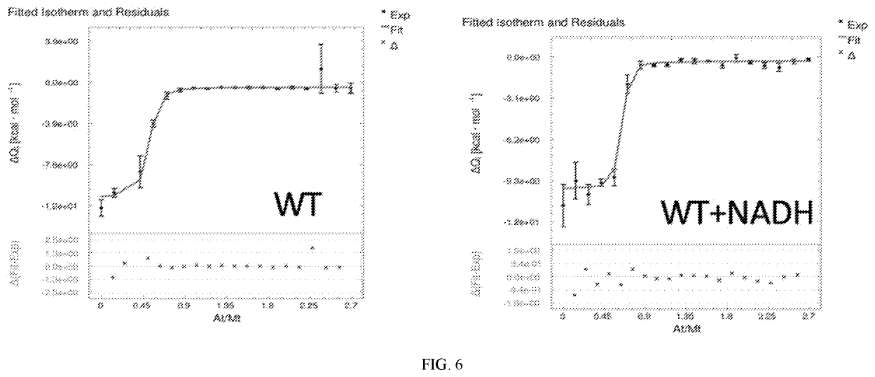

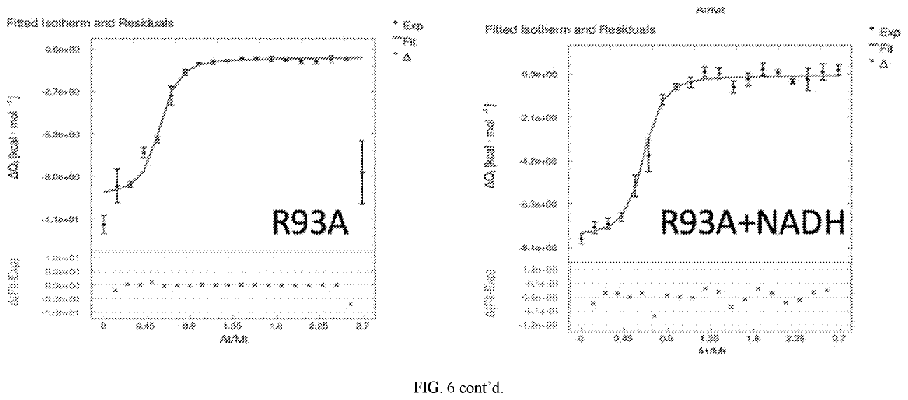

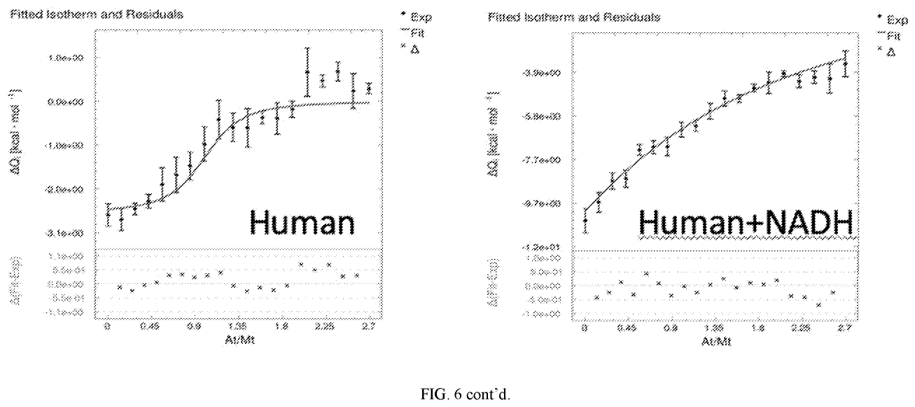

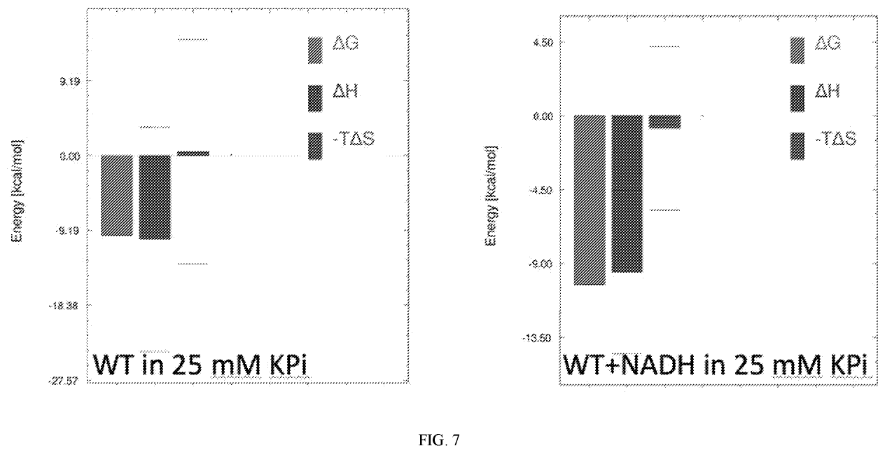

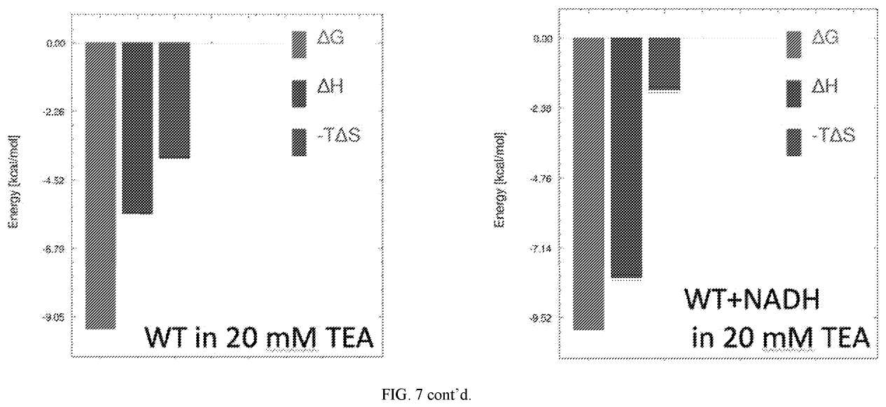

Enzyme kinetics suggested that compound C is a potent inhibitor of Mtb Lpd and that Arg93 is important for this binding. To learn more what was driving this tight association ITC and SPR profiling were performed for both the WT and the R93A Mtb Lpd as well as the human Lpd interaction with compound C (FIG. 5 and FIG. 6). It is observed that the affinity for compound C for WT Mtb Lpd depended on the presence of NADH and was in good agreement with the Kᵢ values calculated through enzyme inhibition assays. Thus, the K_{d} for the WT Lpd decreased more than 2-fold in the presence of NADH, suggesting a possible conformational adjustment within the lipoamide site upon binding of NADH that resulted in a tighter association with compound C (Table 3). The R93A mutant bound compound C in an NADH-independent manner with a K_{d}=227±6.6 nM, which was in agreement with the Kᵢ calculated from enzyme inhibition data. Thus, it appeared that R93 contact contributes to binding affinity and may adjust its conformation upon C and/or NADH binding, which is consistent with its conformational flexibility in the Mtb Lpd crystal structure. The human Lpd bound compound C with much lower affinity and in some repeats we were not able to fit the data properly. Binding of compound C to the WT Mtb Lpd was mostly enthalphy-driven, suggesting hydrogen bonding involvement (FIG. 7).

TABLE 3
Characterization of compound C binding to Lpd by ITC and SPR.

| | ITC | SPR | | | |
|---|---|---|---|---|---|
| | K_{d}, nM | K_{d}, nM | kₒₙ, M⁻¹s⁻¹ | K_{off}, S⁻¹ | t_{1/2}, min |
| Mtb WT | 63.9 ± 2.75 | 52.3 | 35500 | 0.0315 | 0.5 |
| | | | 0.0177 | 0.0011 | 15.15 |
| Mtb WT/ NADH | 28.3 ± 1.74 | 44.5 | 18500 | 0.000827 | 20.15 |
| | | | 0.00000222 | 0.00158 | 10.5 |
| Mtb R93A | 231.4 ± 8.81 | ND | ND | ND | ND |
| Mtb R93A/ NADH | 228.6 ± 2.44 | ND | ND | ND | ND |
| Human WT | 592 | 7600 | 1170 | 0.00887 | 1.9 |
| Human WT/NADH | ND | ND | 110 | 0.00628 | 2.7 |

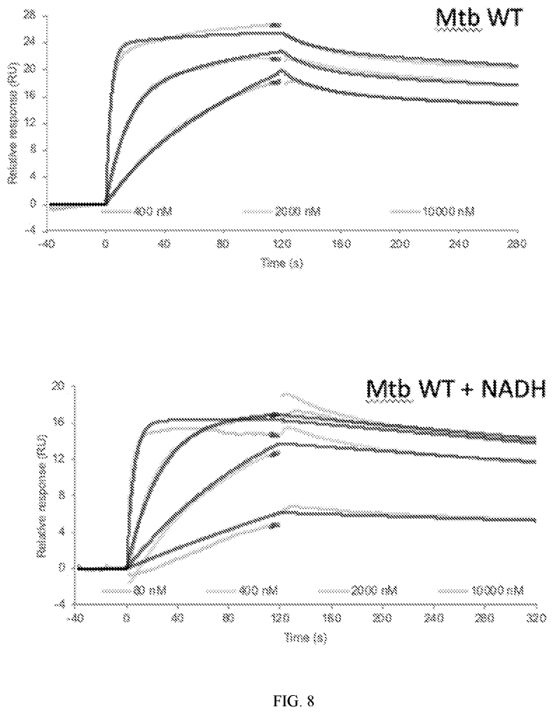

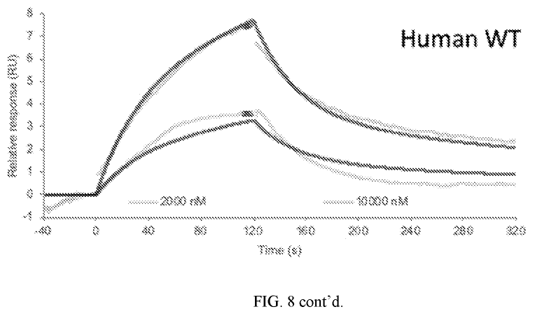

Similarly, kinetic analysis of compound C binding to WT Mtb Lpd by SPR produced K_{d} values close to the ones observed by ITC but we did not observe significant dependence of binding affinity on NADH presence. This could reflect a difference in Lpd native conformation as a dimer, which was preserved in ITC but could have been disrupted in the SPR by covalent immobilization of Lpd. Best fitting of the kinetic binding data was achieved with the two-state model, which had fast on/off and slow on/off components in the absence of NADH. In the presence of NADH the fast on/off component converted to the fast on/slow off site with slow dissociation corresponding to a 40-fold increase in the half-life of the Lpd-compound C complex (Table 3, FIG. 8). The SPR values for k_{off} agreed well with the numbers calculated from rapid dilution experiments. Human Lpd protein produced K_{d} in μM range and faster dissociation rates by SPR with a half-life of 2 minutes, confirming its lower affinity and 10-fold higher dissociation rates for compound C. These results are consistent with our enzymatic data analysis and support the interpretation that Mtb's Lpd binds compound C tightly through an induced fit model in the presence of NADH. This scenario will likely play out inside Mtb in vivo as the mycobacterium maintains high nM to low μM NADH levels throughout different stages of its growth cycle. Human Lpd will be spared from inhibition due to its lower affinity for compound C, aided by rapid dissociation of the human Lpd-compound C complex.

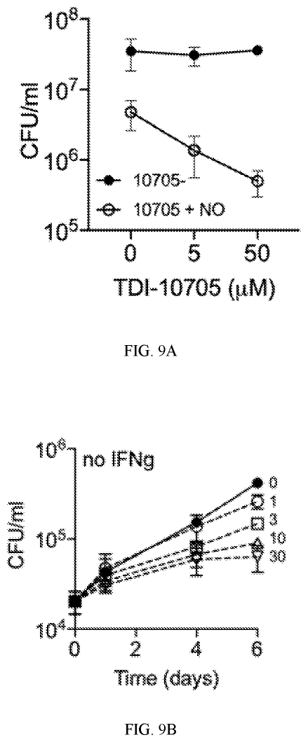

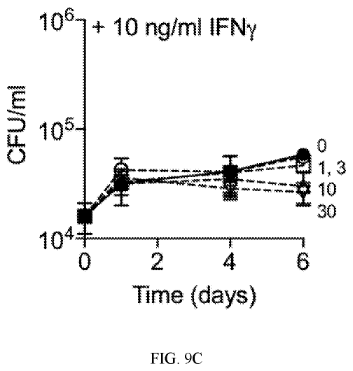

In accordance with high susceptibility of Δlpd Mtb to nitrosative stress, compound C proved to be bactericidal in a concentration-dependent manner to WT Mtb at pH 5.5 in the presence of 3 mM NaNO₂ (which generates a flux of NO at acidic pH) but had no effect at the same concentrations when Mtb was exposed to pH 5.5 alone (FIG. 9A). Mtb Δlpd does not replicate in naïve mouse bone marrow derived macrophages (BMDM) and dies by 1 log₁₀ in BMDM activated by interferon-gama (IFNgama). Compound C inhibited the growth of WT Mtb in naïve BMDM in a concentration-dependent manner, recapitulating the Mtb Δlpd phenotype. By day 6 post infection, treatment with compound C inhibited the growth of Mtb by 1 log₁₀ in naïve BMDM and by 0.5 log₁₀ in IFNgama-activated BMDM at the highest concentration tested, 30 μM. No toxicity was observed to mouse BMDM at any concentration of compound C tested as assessed by the MTS assay, suggesting that the prolonged residence time of compound C on Mtb's Lpd selectively contributed to increased vulnerability of Mtb to Lpd inhibition inside mammalian host cells. Similarly, profiling of compound C in HEPG2 cells showed no signs of cytotoxicity up to 100 μM, consistent with the efficacy in the BMDM experiment being driven through on target activity in Mtb.

### Example 2. Synthesis and Evaluation of Lpd Inhibitor Analogues

### Synthetic Procedures:

### General Method A:

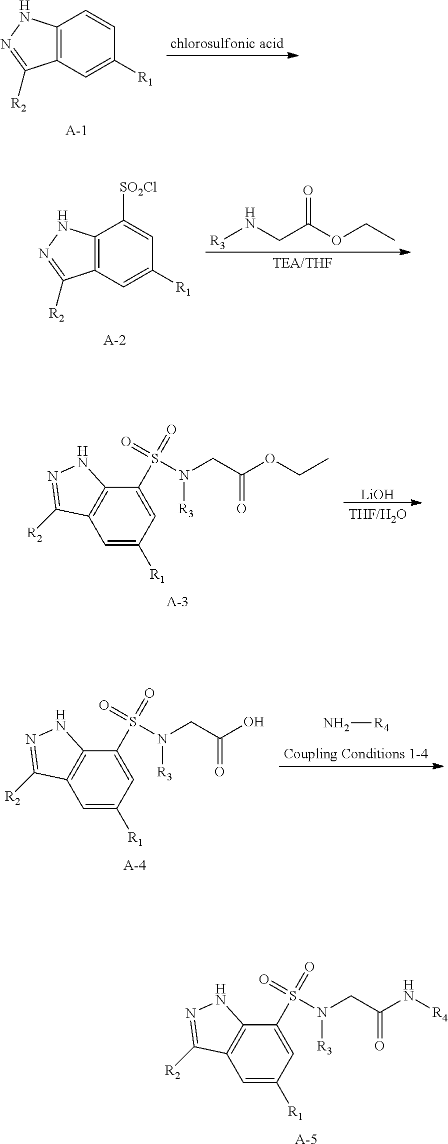

Substituted indazole A-1 is reacted with chlorosulfonic acid to form sulfonyl chloride A-2. Reaction with N-substituted glycine esters and TEA provides sulfonamide A-3. Hydrolysis using lithium hydroxide provides acid intermediate A-4. Acid A-4 can converted to amide A-5 by reaction with NH₃—R₄ under conditions 1-4 outlined as follows:
Condition 1: A solution of A-4 (1 eq), NH₂—R₄ (1.3 eq), and HBTU (1.3 eq) in Pyr. (0.1 M) was stirred at 110 C for 5 hr. The reaction mixture was diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give A-5.
Condition 2: A mixture of A-4 (1 eq), HATU (1.2 eq) and TEA (2.5 eq) in DMF (0.23 M) was stirred at 18° C. for 0.5 hr then NH₂—R₄ (1.2 eq) was added. The mixture was stirred at 18° C. for 2.5 hr. The reaction mixture was diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give A-5.
Condition 3: To a mixture of A-4 (1 eq), NH₂—R₄ (1 eq), DIPEA (3.2 eq) in DMF (0.17 M) was added T₃P (1.3 eq, 50% w/w purity in Ethyl Acetate) at 0° C. The mixture was stirred at 50° C. for 3 hr then diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give A-5.
Condition 4: To a mixture of A-4 (1 eq), NH₃-R₄ (1.5 eq), and PYBOP (2 eq) in DCM (0.035 M) was added TEA (5 eq) at 0° C. under N2, then the mixture was stirred at 40° C. for 22 hr under N2. The reaction mixture was concentrated in vacuum to give a residue. The residue was purified by prep-HPLC (HCl condition or neutral condition) to give A-5.

### General Method B:

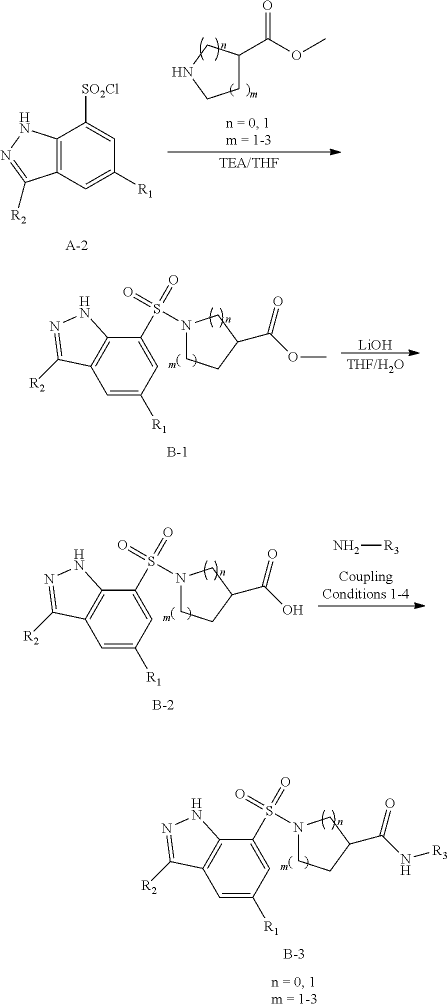

Indazole sulfanyl chloride A-2 is reacted with cyclic amino acid esters and TEA to providid sulfonamides B-1. Hydrolysis using lithium hydroxide provides acid intermediates B-2. Acid B-2 can converted to amide B-3 by reaction with NH₂—R₃ under conditions 1-4 outlined as follows:
Condition 1: To a solution of B-2 (1 eq) in DMF (0.26 M) was added HATU (1 eq) and TEA (2.5 eq). The mixture was stirred at 18° C. for 0.5 hr then NH₂—R₃ (1 eq) was added. The mixture was stirred at 18° C. for 11.5 hr. The reaction mixture was diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give B-3.
Condition 2: To a mixture of B-2 (1 eq), NH₂—R₃ (1 eq), and DIPEA (4.1 eq) in DMF (0.17 M) was added T₃P (1.6 eq, 50% w/w purity in Ethyl Acetate) at 0° C. The mixture was stirred at 50° C. for 3 hr then diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give B-3.
Condition 3: A solution of B-2 (1 eq) in Pyr. (0.13 M) was added HBTU (1.3 eq), the mixture was stirred at 110 C for 0.5 hr, then NH₂—R₃ (1.3 eq) was added. The mixture was stirred at 110 C for 11.5 hr. The reaction mixture was diluted with aq. NaHCO₃ and extracted with EtOAc. The combined organic layers were washed with brine, dried over Na₂SO₄, filtered, and concentrated under reduced pressure to give a residue. The residue was purified by prep-HPLC (neutral condition) to give B-3.
Condition 4: To a solution of B-2 (1 eq), NH₂—R₃ (1 eq) in Pyr. (1 M) was added POCl3 (1 eq) at 0° C. under N₂, then the mixture was stirred at 20° C. for 5 hr under N₂. The reaction mixture was concentrated in vacuum and purified by prep-HPLC (HCl condition) to afford B-3.

### General Method C:

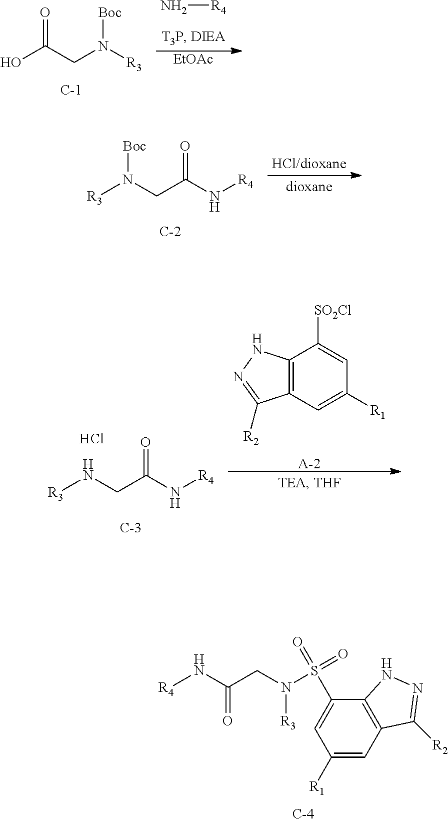

N-protected, N-substituted glycine, C-1, is reacted with NH₂—R₄ using a coupling reagent such as T₃P (50% w/w solution in ethyl acetate) to provide amide C-2. Deprotection using reagents such as HCl in 1,4-dioxane provides amine C-3 as the hydrochloride salt. This can be reacted with indazole sulfonyl chloride A-2 and a base such as TEA to provide sulfonamide C-4.

### General Method D:

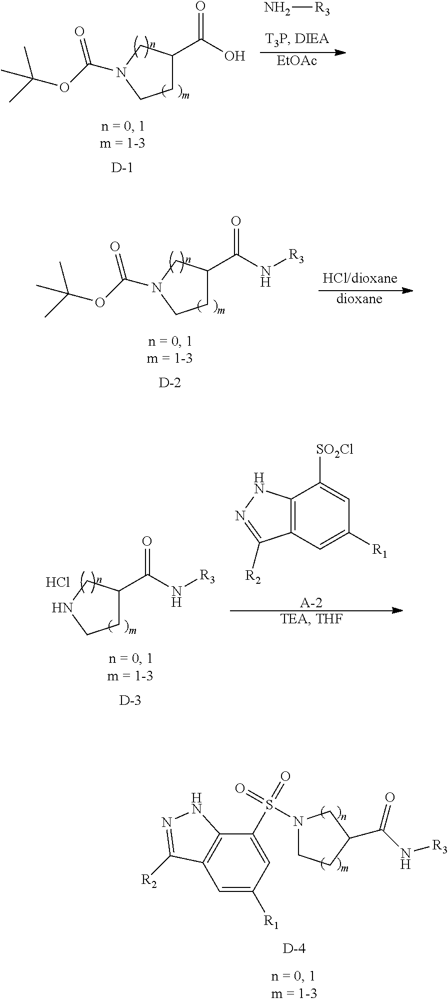

N-protected cyclic amino acid, D-1, is reacted with NH₂—R₄ using a coupling reagent such as T₃P (50% ow/w solution in ethyl acetate) to provide amide D-2. Deprotection using reagents such as HCl in 1,4-dioxane provides amine D-3 as the hydrochloride salt. This can be reacted with indazole sulfonyl chloride A-2 and a base such as TEA to provide sulfonamide D-4.

### General Method E:

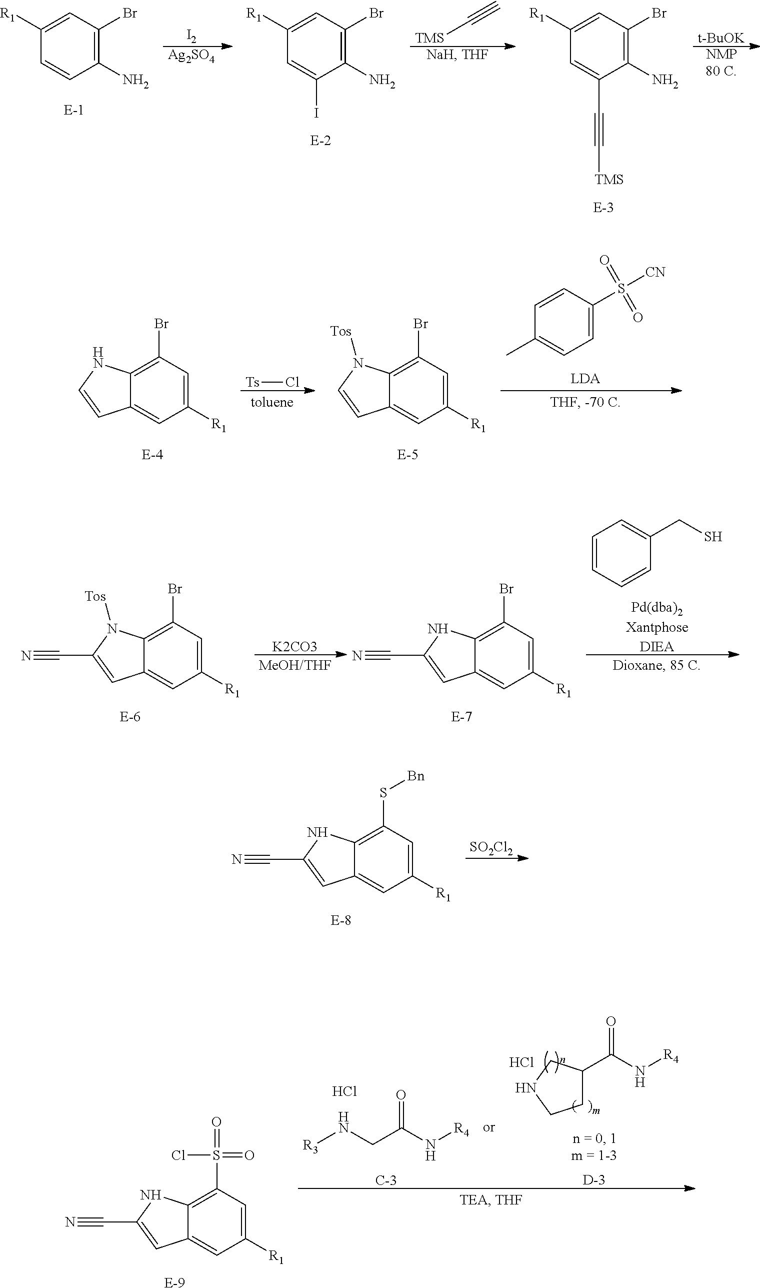

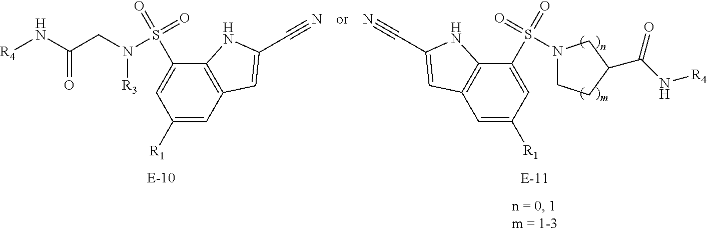

Ortho-bromo aniline, E-1, is reacted with iodine to provide dihalogenated aniline E-2. Sonogashira Coupling with TMS-acetylene provides E-3. Then treatment with potassium tert-butoxide at 80 C leads to the indole E-4. Protection of the indole nitrogen with tosyl chloride leads to E-5. Treatment of E-5 with LDA, at −70 C, followed by p-tolylsulfonylformonitrile provides the 2-cyano indole derivative E-6. Deprotection of E-6 by treatment with potassium carbonate in a mixture of methanol and water provides E-7. Palladium mediated coupling with phenylmethanethiol results in E-8. Treatment of E-8 with thionyl chloride provide the sulfonyl chloride E-9 which can be treated with amine C-3 or D-3 and a base such as triethyl amine to provide the final 2-cyano indole derivatives E-10 or E-11.

### General Method F:

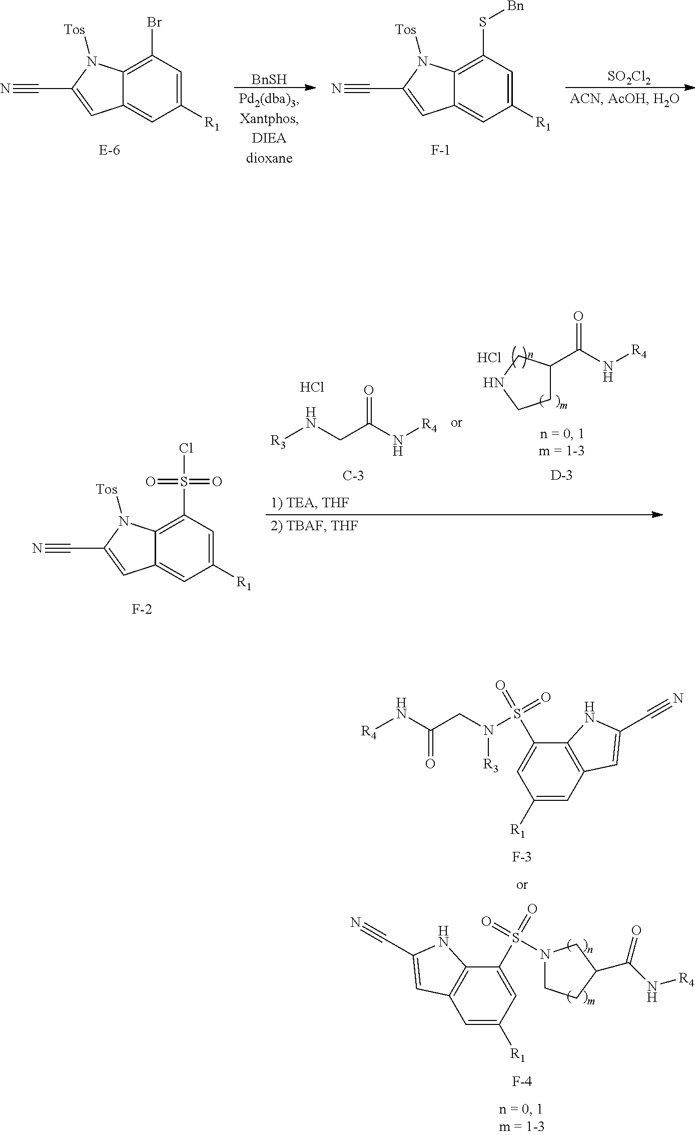

Palladium mediated coupling of E-6 with phenylmethanethiol results in F-1. Treatment of F-1 with thionyl chloride provide the sulfonyl chloride F-2. This can be treated with amine C-3 or D-3 and a base such as triethyl amine followed by deprotection of the tosyl group with TBAF to provide the final 2-cyano indole derivatives F-3 or F-4.

### Intermediates

### Synthesis of 5-methyl-1H-indazole-7-sulfonyl chloride (I-1)

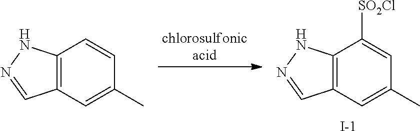

A round bottom flask was charged with chlorosulfonic acid (2 mL) and cooled to 0 C. To this was added 5-methyl-1H-indazole (5a) (1.0 g, 7.6 mmol) slowly at 0° C. The mixture was stirred at 0° C. then warmed to 20° C. within 30 mins. The mixture was then heated to 50 C and stirred for 2 hours. The reaction mixture was poured into ice-water (100 mL) causing a solid to precipitate. The formed solid was collected by filtration and the filter cake was washed with ethyl acetate:tetrahydrofuran (1:1) (100 mL). The filtrate was dried over anhydrous sodium sulfate, filtered, and the filtrate was concentrated under reduced pressure to give 5-methyl-1H-indazole-7-sulfonyl chloride (I-1) (0.98 g, 4.0 mmol, 52% yield) as a brown solid.

LCMS of I-1: m/z: 230.7 [M+H]⁺.

¹H NMR of I-1 (DMSO-d₆, 400 MHz): δ 8.03 (s, 1H), 7.51 (s, 1H), 7.39 (s, 1H), 2.40 (s, 3H).

The following intermediate was synthesized in a similar manner.

| Intermediate | Reactant | LCMS m/z | 1H-NMR |
|---|---|---|---|
| I-14 | 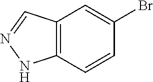 | 296.9 | (CDCl₃, 400 MHz): δ 8.32 (d, J = 1.6 Hz, 1H), 8.26 (s, 1H), 8.17 (d, J = 1.6 Hz, 1H) |
| | 5-bromo-1H- | | |
| | indazole | | |
| I-17 | 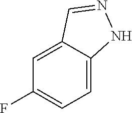 | N/A | (CDCl₃, 400 MHz): δ 8.09 (s, 1H), 7.90- 7.85 (m, 2H) |
| | 5-Fluoro-1H- | | |
| | indazole | | |

### Synthesis of 5-methyl-2-oxoindoline-7-sulfonyl chloride (I-2)

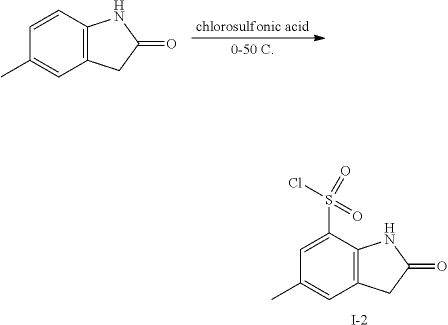

A round bottom flask was charged with chlorosulfonic acid (8.75 g, 75.1 mmol) and cooled to 0° C. To this was added 5-methylindolin-2-one (0.200 g, 1.36 mmol) slowly at 0° C. The mixture was stirred at 0° C., and then warmed to 20° C. within 30 minutes. The mixture was then heated to 50° C. and stirred for 2 hours. The reaction mixture was poured into ice-water (100 mL) and extracted with dichloromethane (50 mL*3). The combined organic layers were washed with brine (100 ml), dried over sodium sulfate, filtered and concentrated under reduce pressure. The residue was purified by prep-TLC (SiO₂, petroleum ether:ethyl acetate=2:1) to afford 5-methyl-2-oxo-indoline-7-sulfonyl chloride (I-2) (0.047 g, 14.0% yield) as a red solid.

¹H-NMR of I-2: (CDCl₃, 400 MHz): δ 8.58 (br. s, 1H), 7.56 (s, 1H), 7.38 (s, 1H), 3.75 (s, 2H), 2.44 (s, 3H)

### Synthesis of N-methyl-N-((5-methyl-1H-indazol-7-yl)sulfonyl)glycine (I-3)

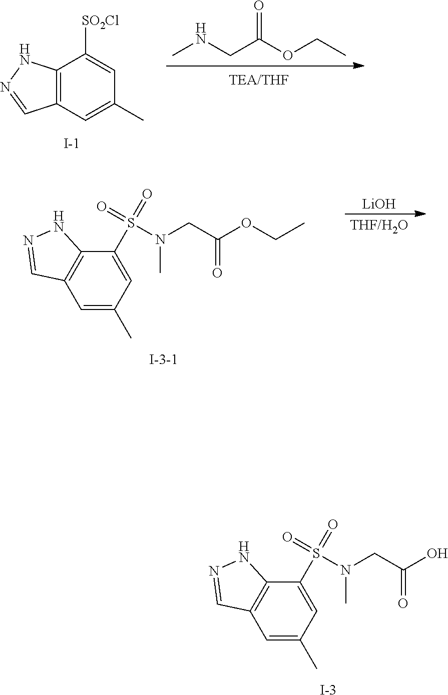

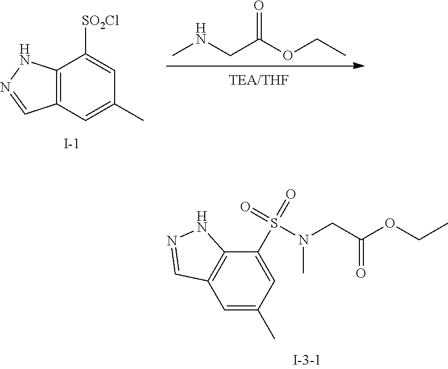

To a solution of I-1 (2.00 g, 7.20 mmol) and trimethylamine (3.00 mL, 21.6 mmol) in THF (20 mL) was added ethyl 2-(methylamino)acetate (1.66 g, 10.8 mmol) at 25° C. The mixture was stirred for 10 hours then poured into water (100 mL) and extracted with ethyl acetate (100 mL*3). The combined organic phase was washed with brine (100 mL), dried over anhydrous sodium sulfate, filtered, and concentrated under reduced pressure. The residue was purified by column chromatography (SiO2, petroleum ether/ethyl acetate=6/1 to 3/1) to afford ethyl 2-(N,5-dimethyl-1H-indazole-7-sulfonamido)acetate (I-3-1) (1.70 g, 72.1% yield) as a yellow oil.

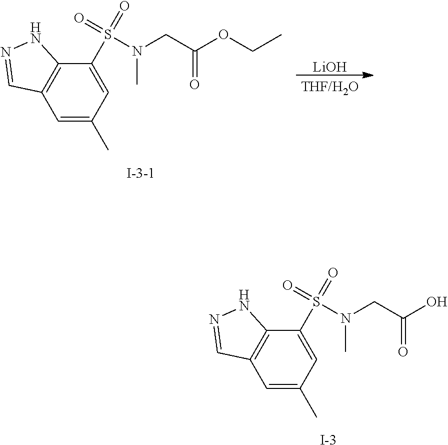

To a solution of I-3-1 (0.900 g, 2.89 mmol) in THF (9 mL) was added a solution of lithium hydroxide monohydrate (0.364 g, 8.67 mmol) in water (4 mL). The mixture was stirred at 25° C. for 2 hours then poured into water (50 mL) and adjusted to pH=5 with a saturated aqueous solution of citric acid. The mixture was extracted with a mixture of DCM/MeOH (v/v=5/1, 50 mL*8). The combined organic phase was washed with brine (50 mL), dried over anhydrous sodium sulfate, filtered, and concentrated in reduce pressure to afford 2-[methyl-[(5-methyl-1H-indazol-7-yl)sulfonyl]amino]acetic acid (I-3) (0.600 g, 73.3% yield) as a white solid.

LCMS of I-3: m/z: 284.1 [M+H]+

¹H-NMR of I-3: (400 MHz, DMSO-d6) δ 13.19 (br. s, 1H), 12.80 (br. s, 1H), 8.17 (s, 1H), 7.88 (s, 1H), 7.62 (d, J=1.2 Hz, 1H), 4.07 (s, 2H), 2.79 (s, 3H), 2.47 (s, 3H).

### Synthesis of N-(3-chloro-4-methoxyphenyl)-2-(methylamino)acetamide (I-4)

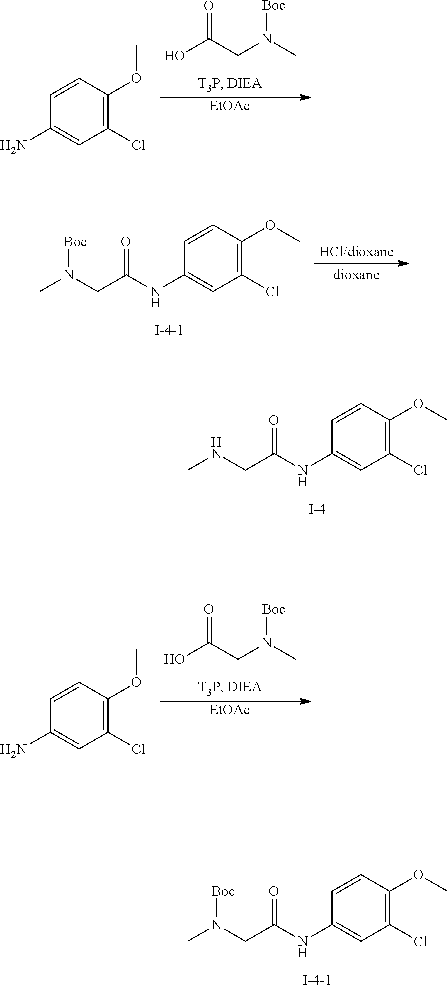

To a solution of 3-chloro-4-methoxyaniline (1.50 g, 9.52 mmol) and N-(tert-butoxycarbonyl)-N-methylglycine (1.98 g, 10.5 mmol) in ethyl acetate (15 mL) was added diisopropyl ethylamine (3.08 g, 23.79 mmol) at 0° C. and followed by T₃P (8.49 mL, 14.3 mmol, 50% w/w solution in ethyl acetate) drop wise. The mixture was stirred at 0° C. for 1.5 hours then poured into ice water (30 mL) and extracted with ethyl acetate (30 mL*4). The combined organic layers were washed with a saturated aqueous solution of sodium bicarbonate (30 mL*2) followed by 1N hydrochloric acid (30 mL*1) and then brine (30 mL*1). The combined organic layers were dried over anhydrous sodium sulfate, filtered, and the filtrate was concentrated under reduced pressure to afford tert-butyl (2-((3-chloro-4-methoxyphenyl)amino)-2-oxoethyl)(methyl)carbamate (I-4-1) (3.20 g, crude)

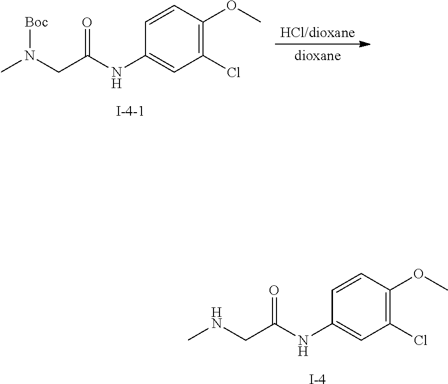

To a solution of I-4-1 (3.80 g, 11.56 mmol) in dioxane (40 mL) was added a solution of HCl in dioxane (4 M, 19 mL) drop wise at 0° C. The mixture was stirred at 25° C. for 2 hours. The reaction mixture was concentrated under reduced pressure to give a residue. The crude product was triturated with ethyl acetate (50 mL) at 25° C. for 20 minutes then filtered and the solid was dried under reduced pressure to afford N-(3-chloro-4-methoxyphenyl)-2-(methylamino)acetamide (I-4) as the hydrochloric salt (2.90 g, 10.94 mmol, 94.6% yield, hydrochloric acid) as a gray solid.

¹H-NMR of I-4: (CDCl₃, 400 MHz): δ 7.74 (d, J=2.4 Hz, 1H), 7.41 (dd, J=2.8, 8.8 Hz, 1H), 7.04 (d, J=9.2 Hz, 1H), 3.95 (s, 2H), 3.86 (s, 3H), 2.79 (s, 3H).

The following intermediates were synthesized in a similar manner.

| | | LCMS | |
|---|---|---|---|
| Intermediate | Reactant | m/z | 1H-NMR |
| I-5 | 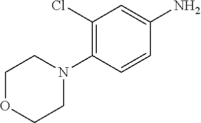 [SMILES: Nc1ccc(N2CCOCC2)c(Cl)c1] | 284.2 | (CD₃OD, 400 MHz): δ 7.99- 7.87 (m, 1H), 7.62-7.51 (m, 1H), 7.48-7.33 (m, 1H), 3.99 (s, 2H), 3.98-3.96 (m, 4H), 3.32 (m, 4H), 2.80 (s, 3H) |
| | 3-chloro-4- | | |
| | morpholinoaniline | | |
| I-11 | 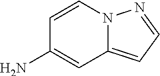 | 205.2 | (DMSO-d₆, 400 MHz): δ 11.17 (br. s, 1H), 9.07 (br. s, 2H), 8.64 (d, J = 7.6 Hz, 1H), 8.09 (d, J = 2.0 Hz, 1H), 7.94 (d, J = 2.0 Hz, 1H), 7.02-6.98 |
| | pyrazolo[1,5-a]pyridin-5- | | (m, 1H), 6.54 (d, J = 1.6 Hz, |
| | amine | | 1H), 4.00 (d, J = 5.2 Hz, 2H), |
| | | | 2.63 (td, J = 5.2, 2.4 Hz, 3H) |
| I-12 | 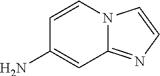 | — | (DMSO-d₆, 400 MHz) δ 14.28 (br. s, 1H), 12.21 (br. s, 1H), 9.20 (br. s, 2H), 8.83 (d, J = 7.2 Hz, 1H), 8.41 (d, J = 2.0 Hz, 1H), 8.24 (d, J = 1.6 Hz, |
| | imidazo[1,2-a]pyridin-7- | | 1H), 8.07 (d, J = 2.4 Hz, 1H), |
| | amine | | 7.60 (dd, J = 7.2, 2.0 Hz, 1H), |
| | | | 4.09 (s, 2H), 2.64 (s, 3H) |
| I-13 | 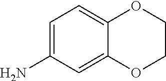 [SMILES: Nc1ccc2c(c1)OCCO2] | 223.1 | (DMSO-d6, 400 MHz): δ 10.35 (br. s, 1H), 8.80 (br. s, 2H), 7.21 (s, 1H), 6.95 (d, J = 8.4 Hz, 1H), 6.83 (d, J = 8.8 Hz, 1H), 4.25-4.19 (m, 4H), |
| | 2,3- | | 3.87 (s, 2H), 2.61 (s, 3H) |
| | dihydrobenzo[b][1,4]dioxin- | | |
| | 6-amine | | |
| I-16 | 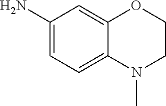 | | |
| | 4-methyl-3,4-dihydro-2H- | | |
| | benzo[b][1,4]oxazin-7- | | |
| | amine | | |
| I-26 | 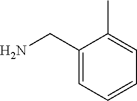 | N/A | (CDCl₃, 400 MHz): δ 8.96- 8.88 (br. m, 3H), 7.24-7.22 (m, 1H), 7.17-7.14 (m, 3H), 4.30 (d, J = 5.6 Hz, 2H), 3.74 (s, 2H), 2.54 (s, 3H), 2.27 (s, 3H) |
| | o-tolylmethanamine | | |
| I-27 | 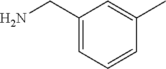 | 193.3 | (DMSO, 400 MHz): δ 9.05(br.s, 3H), 7.18(t, J = 7.6 Hz 1H), 7.07-7.04(m, 3H), 4.27(d, J = 6.0 Hz, 2H), 3.71(t, J = 6.0 Hz, 2H), 3.53(s, 3H), |
| | m-tolylmethanamine | | 2.25(s, 3H) |
| I-28 | 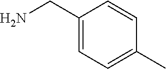 | 193.3 | (CDCl3, 400 MHz): δ 8.74 (br. s, 2H), 7.29 (br. s, 1H), 7.14-7.09 (m, 2H), 7.09- 7.02 (m, 2H), 4.35 (d, J = 4.8 Hz, 2H), 3.87 (s, 3H), 2.77 |
| | p-tolylmethanamine | | (s, 3H) |
| I-29 | 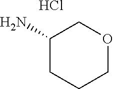 | 173.3 | (CDCl3, 400 MHz) δ 8.94 (br. s, 2H), 7.37-7.29 (m, 1H), 4.02-3.93 (m, 1H), 3.88 (s, 2H), 3.80-3.59 (m, 3H), 3.53- 3.42 (m, 1H), 2.84 (s, 3H), |
| | (S)-tetrahydro-2H-pyran-3- | | 1.93-1.52 (m, 4H) |
| | amine hydrochloride salt | | |
| I-30 | 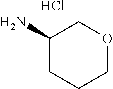 | 173.4 | (DMSO-d6, 400 MHz): δ 8.71 (br. s, 2H), 8.45 (d, J = 7.2 Hz, 1H), 3.83-3.55 (m, 5H), 3.47- 3.33 (m, 1H), 3.26-3.11 (m, 1H), 2.55 (s, 3H), 1.90-1.78 |
| | (R)-tetrahydro-2H-pyran-3- | | (m, 1H), 1.73-1.64 (m, 1H), |
| | amine hydrochloride salt | | 1.58-1.41 (m, 2H) |
| I-35 | I-15 | | |
| I-37 | I-18 | | |
| I-42 | I-41 | 306.0 | |
| I-56 | 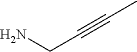 | | (DMSO-d₆, 400 MHz) δ = 9.00-8.84 (m, 3H), 3.90 (s, 2H), 3.67 (s, 2H), 2.54 (s, 3H), 1.77 (s, 3H) |
| | but-2-yn-1-amine | | |
| I-61 | 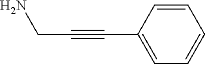 | 203.3 | |
| | 3-phenylprop-2-yn-1-amine | | |
| I-62 | 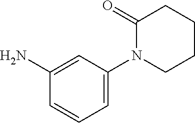 [SMILES: Nc1cccc(N2CCCCC2=O)c1] | | (DMSO-d6, 400 MHz) δ 10.81 (br. s, 1H), 9.02 (d, J = 4.8 Hz, 2H), 7.55-7.54 (m, 1H), 7.47 (d, J = 8.0 Hz, 1H), 7.35 (t, J = 8.0 Hz, 1H), 7.02-7.00 (m, 1H), 3.92 (t, J = 5.6 Hz, 2H), 3.57 (s, 2H), 2.61 (t, J = 5.2 Hz, 3H), 2.39 (t, J = 6.4 Hz, |
| | 1-(3- | | 2H), 1.86-1.83 (m, 4H) |
| | aminophenyl)piperidin-2- | | |
| | one | | |
| I-63 | 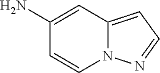 | 205.1 | |
| | pyrazolo[1,5-a]pyridin-5- | | |
| | amine | | |
| I-64 | 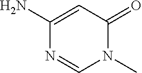 | | (DMSO-d₆, 400 MHz) δ 11.00 (s, 1H), 9.13 (s, 2H), 8.43 (s, 1H), 6.90 (s, 1H), 3.94 (s, 2H), 3.36 (s, 3H), 2.58 (s, 3H) |
| | 6-amino-3- | | |
| | methylpyrimidin-4(3H)-one | | |
| I-65 | 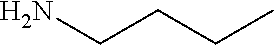 | | |
| | butan-1-amine | | |
| I-70 | 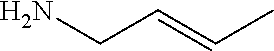 | | |
| | (E)-but-2-en-1-amine | | |
| I-71 | 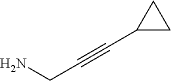 | | |
| | 3-cyclopropylprop-2-yn-1- | | |
| | amine | | |

### Synthesis of N-(1-methyl-2-oxo-1,2-dihydropyridin-4-yl)-2-(methylamino)acetamide hydrochloride (1-6)

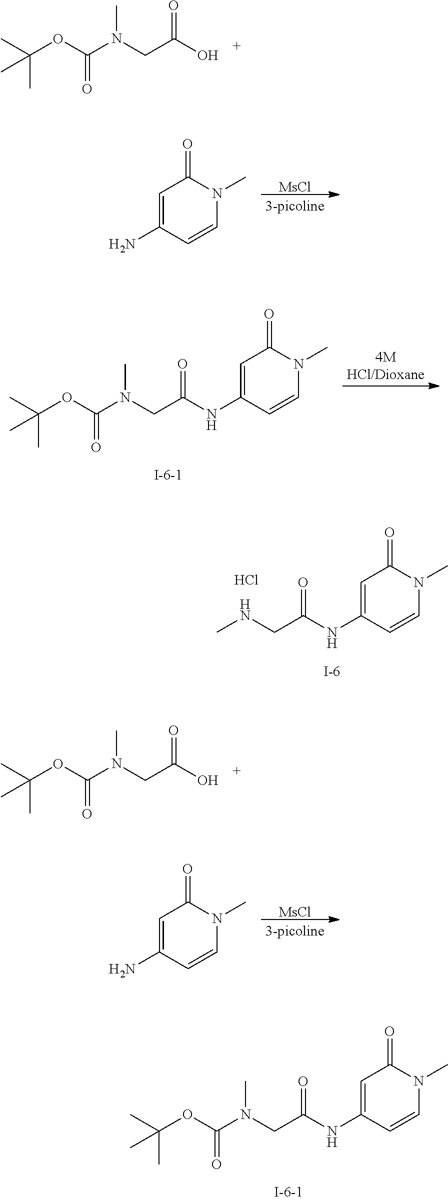

To a solution of 2-[tert-butoxycarbonyl(methyl)amino]acetic acid (0.553 g, 2.82 mmol) and 4-amino-1-methyl-pyridin-2-one (0.350 g, 2.82 mmol) in 3-picoline (4 mL) at 0° C. was added methanesulfonyl chloride (0.646 g, 5.64 mmol). The mixture was stirred at 0° C. for 11 hours, then poured into water (10 mL) and adjust to approximately pH˜8 with a saturated aqueous solution of sodium bicarbonate solution (20 mL). The mixture was extracted with ethyl acetate (10 mL*3), the combined organic layers were washed with brine (20 mL), dried over anhydrous sodium sulfate, filtered, and concentrated under reduced pressure. The residue was purified by column chromatography (SiO₂, petroleum ether/ethyl acetate=20/1 to methanol) to afford tert-butyl N-methyl-N-[2-[(1-methyl-2-oxo-4-pyridyl)amino]-2-oxo-ethyl]carbamate (I-6-1) (0.300 g, 36.0% yield) as a red solid.

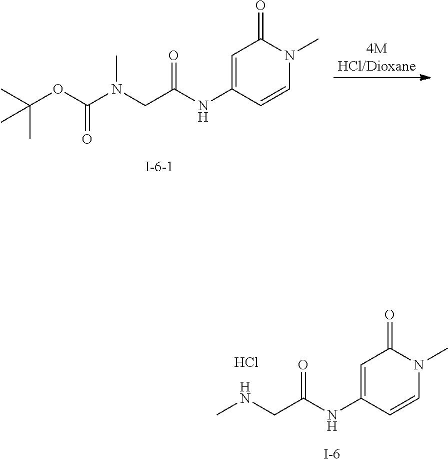

To a solution of I-6-1 (0.300 g, 1.02 mmol) in 1,4-dioxane (4 mL) was added a solution of HCl in 1,4-dioxane (4 M, 2 mL) at 25° C. The mixture was stirred for 10 hours, then concentrated under reduced pressure to give 2-(methylamino)-N-(1-methyl-2-oxo-4-pyridyl) acetamide hydrochloride salt (I-6) (0.235 g, 99.9% yield) as a yellow solid. No additional purification was performed.

¹H-NMR of I-6 (400 MHz, Methanol-d4) δ 7.99 (d, J=7.2 Hz, 1H), 7.51 (d, J=2.4 Hz, 1H), 7.04 (dd, J=7.2, 2.4 Hz, 1H), 4.09 (s, 2H), 3.75 (s, 3H), 2.80 (s, 3H)

The following intermediates were synthesized in a similar manner as I-6 using the reactant indicated in replace of 4-amino-1-methyl-pyridin-2-one.

| | | LCMS | |
|---|---|---|---|
| Intermediate | Reactant | m/z | 1H-NMR |
| I-22 | 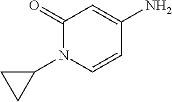 [SMILES: Nc1ccn(C2CC2)c(=O)c1] | 236.1 | (CD₃OD, 400 MHz) δ 8.08-7.84 (m, 1H), 7.74-7.40 (m, 1H), 7.23-6.89 (m, 1H), 3.80-3.68 (m, 3H), 3.66 (s, 3H), 3.50-3.36 (m, 1H), 3.15-2.97 (m, 2H), 2.24-2.09 (m, 1H), 2.07- 1.72 (m, 4H), 1.40-1.36 (m, 1H) |
| | 4-amino-1- | | |
| | cyclopropylpyridin- | | |
| | 2(1H)-one | | |

### Synthesis of N-(1-methyl-2-oxo-1,2-dihydropyridin-4-yl)-2-(methylamino)acetamide (I-7)

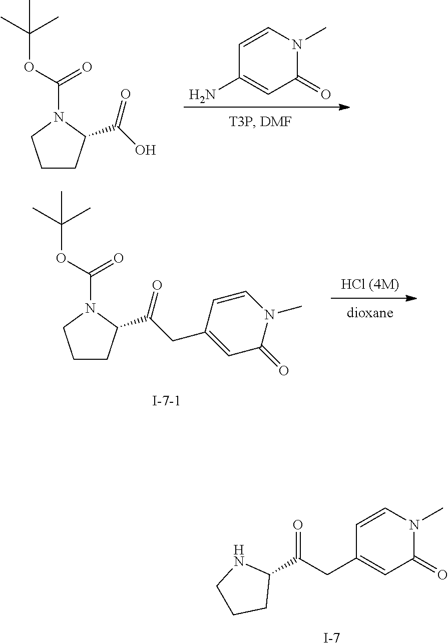

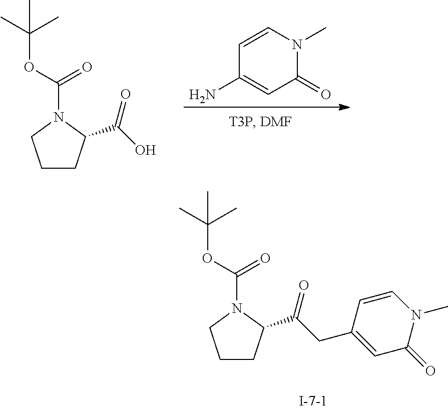

To a solution of (S)-1-(tert-butoxycarbonyl)pyrrolidine-2-carboxylic acid (1.04 g, 4.83 mmol) and 4-amino-1-methylpyridin-2(1H)-one (0.500 g, 4.03 mmol) in dimethyl formamide (5 mL) was added diisopropylethylamine (2.10 mL, 12.08 mmol), then T₃P (6.04 mmol, 3.59 mL, 50% solution in ethyl acetate) was added drop-wise at 20° C. The mixture was stirred for 12 hours at 50° C. under nitrogen atmosphere. The mixture was diluted with water (8 mL) and extracted with ethyl acetate (30 mL*3). The organic layer was washed with a saturated aqueous solution of sodium bicarbonate (20 mL) followed by a saturated aqueous solution of citric acid (20 mL) and brine (20 mL*2), then dried over anhydrous sodium sulfate, filtered, and the filtrate was concentrated under reduced pressure to afford (S)-tert-butyl2-((1-methyl-2-oxo-1,2-dihydropyridin-4-yl)carbamoyl)pyrrolidine-1-carboxylate (I-7-1) (0.150 g, 10.9% yield) as a yellow solid.

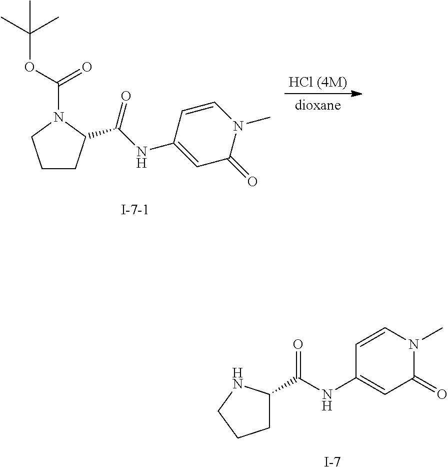

A round bottom flask was charged with a solution of HCl in 1,4-dioxane (4 M, 2.00 mL, 8.00 mmol). To this was added I-7-1 (0.140 g, 0.436 mmol). The mixture was stirred for 0.5 hour at 0-20° C. under nitrogen then concentrated under reduced pressure to afford (S)—N-(1-methyl-2-oxo-1, 2-dihydropyridin-4-yl)pyrrolidine-2-carboxamide hydrochloride (I-7) (0.110 g, crude) as a white solid. No additional purification was performed.

The following intermediates were synthesized in a similar manner as I-7 replacing 4-amino-1-methylpyridin-2(1H)-one with the reactant indicated.

| | | LCMS | |
|---|---|---|---|
| Intermediate | Reactant | m/z | 1H-NMR |
| I-20 | 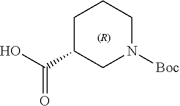 | 236.1 | (CD₃OD, 400 MHz) δ 8.−8-7.84 (m, 1H), 7.−4-7.40 (m, 1H), 7.−3-6.89 (m, 1H), 3.−0-3.68 (m, 3H), 3.66 (s, 3H), 3.−0-3.36 (m, 1H), 3.−5-2.97 (m, 2H), 2.−4-2.09 (m, 1H), 2.−7- 1.72 (m, 4H), 1.−0-1.36 (m, 1H) |
| | I-1-(tert- | | |
| | butoxycarbonyl)piperidine- | | |
| | 3-carboxylic acid | | |
| I-21 | 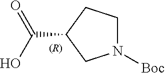 | | |
| | (R)-1-(tert- | | |
| | butoxycarbonyl)pyrrolidine- | | |
| | 3-carboxylic acid | | |
| I-57 | 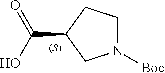 | 222.0 | (DMSO-d₆, 400 MHz) δ 10.71 (br. s, 1H), 7.65 (d, J = 7.2 Hz, 1H), 6.89 (d, J = 2.4 Hz, 1H), 6.48 (dd, J₁ = 2.4 Hz, J₂ = 7.2 Hz, 1H), 3.−7-3.94 (m, 1H), 3.54 (s, 3H), 3.31 (d, J = 7.0 Hz, 2H), |
| | (S)-1-(tert- | | 3.−1-3.18 (m, 2H), 2.−6-2.20 (m, |
| | butoxycarbonyl)pyrrolidine- | | 1H), 2.−5-2.00 (m, 1H) |
| | 3-carboxylic acid | | |

### Synthesis of 3-ethyl-4-morpholinoaniline (I-8)

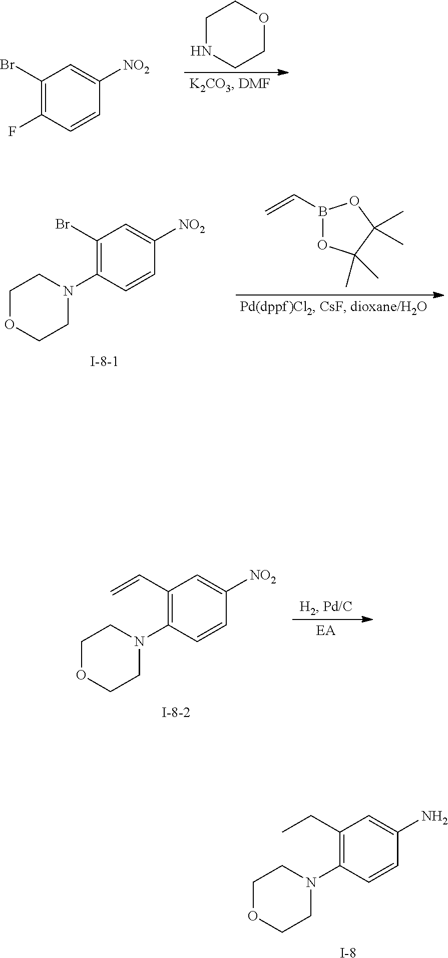

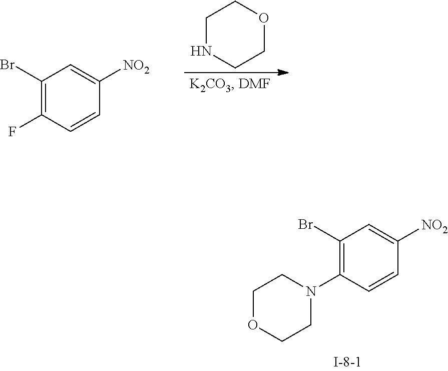

To a solution of 2-bromo-1-fluoro-4-nitrobenzene (1.00 g, 4.55 mmol) in dimethyl formamide (10 mL) was added morpholine (0.600 mL, 6.82 mmol) and potassium carbonate (1.88 g, 13.6 mmol) at 25° C. The mixture was stirred at 70° C. for 3 hours, then poured into water (50 mL), causing a solid to precipitate out. The formed solid was collected by filtration and the filter cake was rinsed with water (10 mL*2) to afford 4-(2-bromo-4-nitrophenyl)morpholine (I-8-1) (1.31 g, 100% yield) as a yellow solid. No further purification was performed.

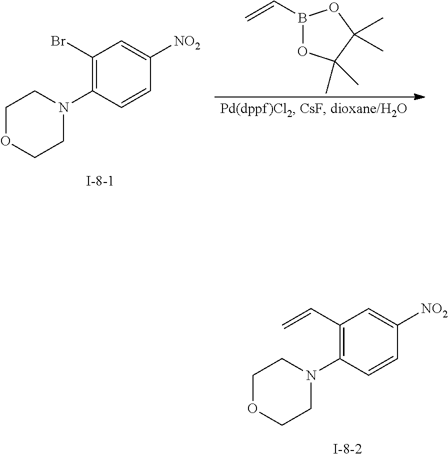 [SMILES: O=[N+]([O-])c1ccc(N2CCOCC2)c(Br)c1 ; C=CB1OC(C)(C)C(C)(C)O1 ; C=Cc1cc([N+](=O)[O-])ccc1N1CCOCC1]

To a solution of I-8-1 (0.500 g, 1.74 mmol), 4,4,5,5-tetramethyl-2-vinyl-1,3,2-dioxaborolane (0.324 mL, 1.92 mmol), and cesium fluoride (0.529 g, 3.48 mmol) in dioxane (5 mL) and water (0.5 mL) was added Pd(dppf)Cl₂ (0.127 g, 0.174 mmol) under nitrogen. The mixture was degassed and purged with nitrogen three times and stirred at 85° C. for 12 hours under nitrogen atmosphere. After being cooled to room temperature, the mixture was concentrated under reduced pressure. The residue was purified by column chromatography (SiO₂, petroleum ether/ethyl acetate=50/1) to afford 4-(4-nitro-2-vinylphenyl)morpholine (I-8-2) (0.240 g, 0.942 mmol, 92.0% purity) as a yellow solid. No further purification was performed.

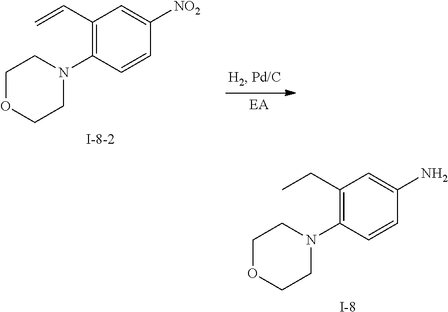

A solution of I-8-2 (0.100 g, 0.427 mmol) in ethyl acetate (1 mL) was degassed and purged with nitrogen three times. Pd/C (0.010 g, 10% purity on charcoal, wet) was added in one portion. The result mixture was degassed purged with hydrogen three times and stirred at 25° C. for 3 hours under a hydrogen atmosphere (15 psi). The mixture was filtered and the filtrate was concentrated under reduced pressure to afford 3-ethyl-4-morpholinoaniline (I-8) (0.106 g, 96.5% yield) as a yellow solid. No further purification was performed.

LCMS of I-8: RT=m/z 207.3 [M+H]+

¹H-NMR of I-8: (CDCl₃, 400 MHz) δ 6.95 (d, J=8.4 Hz, 1H), 6.59 (d, J=2.8 Hz, 1H), 6.53 (dd, J=8.0, 2.8 Hz, 1H), 3.-3-3.81 (m, 4H), 3.53 (br. s, 2H), 2.-3-2.81 (m, 4H), 2.66 (q, J=7.2 Hz, 2H), 1.21 (t, J=7.6 Hz, 3H).

### Synthesis of 3-cyclopropyl-4-morpholinoaniline (I-9)

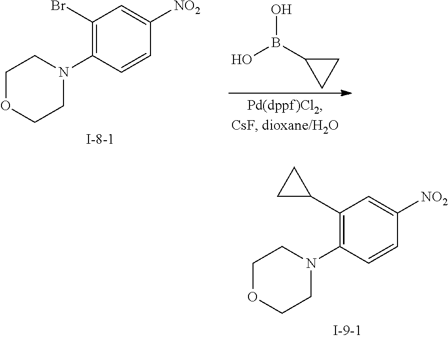 [SMILES: O=[N+]([O-])c1ccc(N2CCOCC2)c(Br)c1 ; OB(O)C1CC1 ; O=[N+]([O-])c1ccc(N2CCOCC2)c(C2CC2)c1]

To a solution of I-8-1 (0.500 g, 1.74 mmol) and cyclopropylboronic acid (0.224 g, 2.61 mmol) in 1,4-dioxane (5 mL) was added a solution of cesium fluoride (0.529 g, 3.48 mmol) in water (0.5 mL). The mixture was degassed and purged with nitrogen, then Pd(dppf)Cl₂ (0.127 mg, 0.174 mmol) was added at 25° C. under a nitrogen atmosphere. The mixture was stirred at 85° C. for 12 hours under a nitrogen atmosphere. The mixture was filtered and the filtrate was concentrated under reduced pressure. The residue was purified by column chromatography (SiO₂, petroleum ether/ethyl acetate=50/1˜10:1) to afford 4-(2-cyclopropyl-4-nitrophenyl)morpholine (I-9-1) (0.334 g, 74.2% yield) as a yellow solid. No further purification was performed.

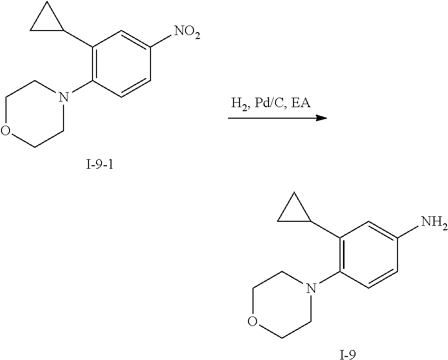

A solution of I-9-1 (0.100 g, 0.403 mmol) in ethyl acetate (1 mL) was degassed and purged with nitrogen three times, then Pd/C (0.010 g, 10% purity on charcoal, wet) was added. The mixture was degassed and purged with hydrogen three times then stirred at 25° C. for 3 hours under a hydrogen atmosphere (15 psi). The mixture was filtered and the filtrate was concentrated under reduced pressure to afford 3-cyclopropyl-4-morpholinoaniline (I-9) (0.085 g, 84.2% yield) as a yellow solid. No further purification was performed.

LCMS of I-9: m/z 219.2 [M+H]+

¹H-NMR of I-9: (CDCl₃, 400 MHz) δ 6.88 (d, J=8.4 Hz, 1H), 6.49 (dd, J=8.4, 2.8 Hz, 1H), 6.11 (d, J=2.8 Hz, 1H), 3.-6-3.84 (m, 4H), 3.47 (br. s, 2H), 2.-5-2.93 (m, 4H), 2.-9-2.36 (m, 1H), 0.-8-0.93 (m, 2H), 0.-7-0.64 (m, 2H)

### Synthesis of N-(3-chloro-4-morpholinophenyl)-2-(ethylamino)acetamide dihydrochloride salt (I-10)

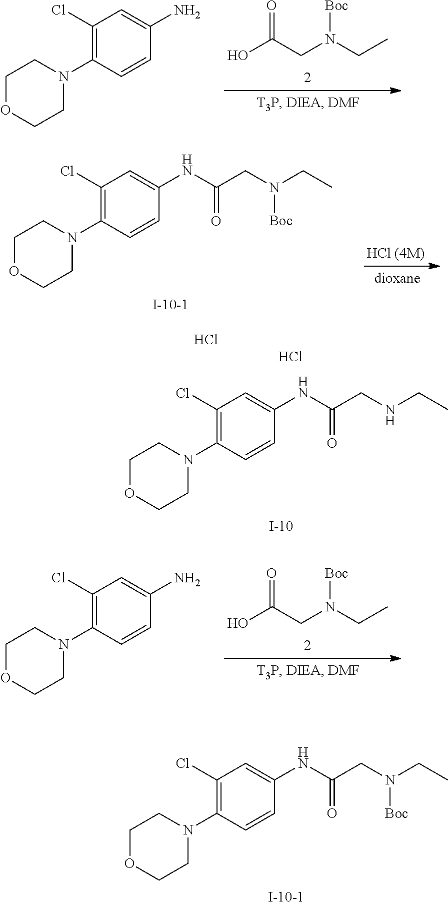

To a solution of 2-((tert-butoxycarbonyl)(ethyl)amino)acetic acid (0.500 g, 2.46 mmol), 3-chloro-4-morpholinoaniline (0.523 g, 2.46 mmol) and diisopropylethylamine (1.29 mL, 7.38 mmol) in dimethylformamide (5 mL) was added T₃P (2.19 mL, 3.69 mmol, 50% purity in ethyl acetate) drop wise at 0° C. The reaction mixture was stirred at 20° C. for 1 hour under nitrogen. The reaction was quenched with a saturated aqueous solution of ammonium chloride (20 mL), extracted with ethyl acetate (100 mL*2), washed with brine (30 mL), and dried over anhydrous sodium sulfate. The mixture was filtered and the filtrate was concentrated under reduced pressure to give tert-butyl (2-((3-chloro-4-morpholinophenyl)amino)-2-oxoethyl)(ethyl)carbamate (I-10-1) (0.850 g, 86.8% yield) as yellow gum, which was used directly without further purification.

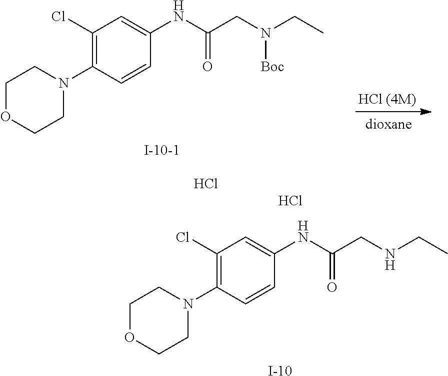

To a solution of I-10-1 (0.300 g, 0754 mmol) in dioxane (1 mL) was added a solution of HCl in dioxane (4 M, 2 mL, 8 mmol) at 20° C. and the reaction mixture was stirred at 20° C. for 1 hour. The reaction was concentrated under reduced pressure to give the di-hydrochloric salt N-(3-chloro-4-morpholinophenyl)-2-(ethylamino)acetamide (I-10) (0.22 g, 87.3% yield) as a yellow solid, which was used directly without further purification.

LCMS of I-10: m/z 298.1 [M+H]⁺ (Method B)

¹H-NMR of I-10: (DMSO-d6, 400 MHz): δ 9.14 (br. s, 2H), 7.79 (d, J=2.4 Hz, 1H), 7.51 (dd, J=8.8, 2.4 Hz, 1H), 7.18 (d, J=8.8 Hz, 1H), 5.98 (br. s, 2H), 3.-5-3.92 (m, 2H), 3.-4-3.72 (m, 4H), 2.95 (q, J=7.2 Hz, 2H), 2.-4-2.89 (m, 4H), 1.22 (t, J=7.2 Hz, 3H)

### Synthesis of 4-(tetrahydro-2H-pyran-4-yl)-3,4-dihydro-2H-benzo[b][1,4]oxazin-7-amine (I-15)

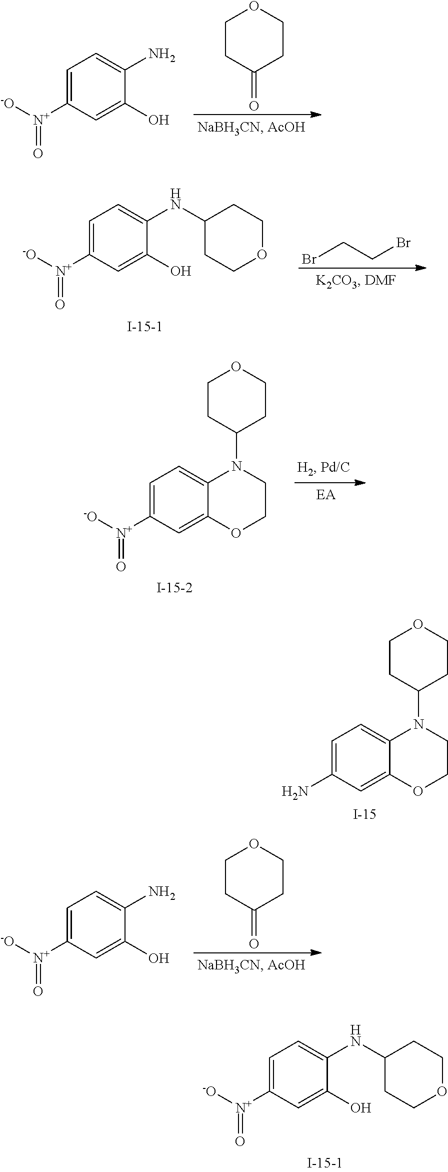

To a solution of 2-amino-5-nitro-phenol (0.100 g, 0.649 mmol) in acetic acid (1 mL) was added tetrahydropyran-4-one (0.520 g, 5.19 mmol). The mixture was stirred at 20° C. for 1 hour then sodium cyanoborohydride (0.204 g, 3.24 mmol) was added and the mixture stirred at 20° C. for 12 hours. The mixture was poured into 25 mL of water, adjusted to pH=8 with sodium bicarbonate, then extracted with ethyl acetate (20 mL*3). The combined organic layer was washed with brine (20 mL*2), dried over anhydrous sodium sulfate, filtered and the filtrate was concentrated under reduced pressure. The residue was triturated with ethyl acetate/petroleum ether (1/5) to afford 5-nitro-2-((tetrahydro-2H-pyran-4-yl)amino)phenol (I-15-1) (0.110 g, 0.439 mmol, 67.6% yield) as a brown solid.

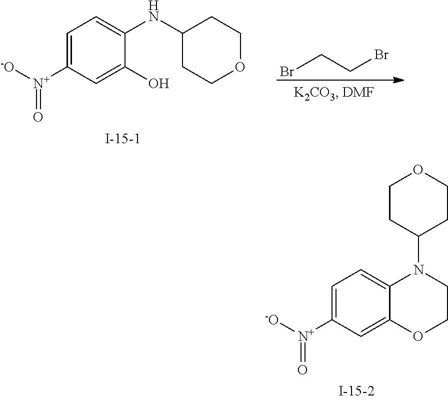

To a solution of I-15-1 (0.150 g, 0.630 mmol) in dimethylformamide (2 mL) was added potassium carbonate (0.174 g, 1.26 mmol), followed by 1, 2-dibromoethane (0.142 g, 0.756 mmol). The mixture was stirred at 65° C. for 12 hours. The mixture was poured into 50 mL of water, causing a solid to precipitate out, the solid was collected by filtration to afford 7-nitro-4-(tetrahydro-2H-pyran-4-yl)-3,4-dihydro-2H-benzo[b][1,4]oxazine (I-15-2) (0.150 g, 0.498 mmol, 79.1% yield) as a yellow solid.

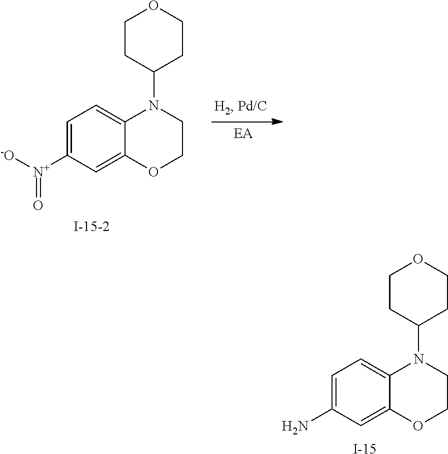

A mixture of I-15-2 (0.150 g, 0.568 mmol) in ethyl acetate (3 mL) was degassed and purged with nitrogen for three times, Pd/C (0.020 g, 10% purity on activate carbon) was added. The mixture was degassed with hydrogen for three times, the result mixture was stirred under hydrogen atmosphere (15 psi) at 20° C. for 5 hours. The mixture was filtered and the filtrate was concentrated under reduced pressure to afford 4-(tetrahydro-2H-pyran-4-yl)-3,4-dihydro-2H-benzo[b][1,4]oxazin-7-amine (I-15) (0.050 g, 0.213 mmol, 37.6% yield) as brown oil.

LCMS of I-15: m/z 234.2 [M+H]⁺

¹H-NMR of I-15: (CDCl₃, 400 MHz): δ 6.-4-6.59 (m, 1H), 6.-8-6.22 (m, 2H), 4.-5-4.18 (m, 2H), 4.-3-4.05 (m, 2H), 3.-4-3.66 (m, 1H), 3.-9-3.44 (m, 2H), 3.-3-3.15 (m, 2H), 1.-2-1.71 (m, 4H)

The following intermediate was synthesized in a similar manner as I-15 using the reacant indicated.

| | | LCMS | |
|---|---|---|---|
| Intermediate | Reactant | m/z | 1H-NMR |
| I-41 | 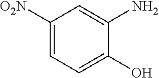 [SMILES: Nc1cc([N+](=O)[O-])ccc1O] | 235.0 | (400 MHz, DMSO-d₆) δ 6.35 (d, J = 8.4 Hz, 1H), 6.15 (d, J = 2.0 Hz, 1H), 5.77 (dd, J = 8.4, 2.4 Hz, 1H), 4.36 (br. s, 2H), 4.−0-3.93 (m, 4H), 3.−3- 3.67 (m, 1H), 3.−3-3.36 (m, 2H), 3.− |
| | 2-amino-4-nitrophenol | | 7-3.15 (m, 2H), 1.−4-1.64 (m, 2H), |
| | | | 1.−0-1.56 (m, 2H) |

### Synthesis of 4-(oxetan-3-yl)-3,4-dihydro-2H-benzo[b][1,4]oxazin-7-amine (I-18)

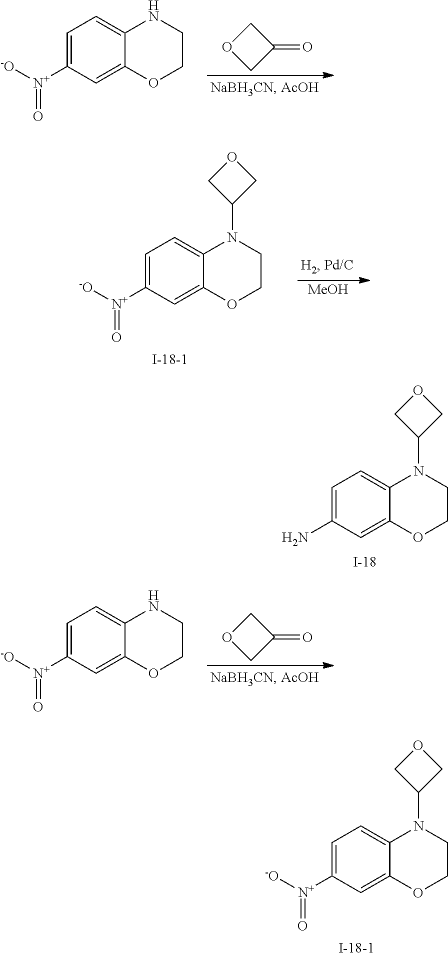

To a solution of 7-nitro-3,4-dihydro-2H-benzo[b][1,4]oxazine (0.500 g, 2.78 mmol) in acetic acid (5 mL) was added oxetan-3-one (4.00 g, 55.5 mmol). The mixture was stirred at 50° C. for 12 hours. Then to the mixture was added NaBH₃CN (0.900 g, 14.3 mmol) in portions. The mixture was stirred at 25° C. for 24 hours. The residue was dissolved in water (50 mL), ethyl acetate (50 mL) was added, then extracted with ethyl acetate (50 mL*2). The combined organic phase was washed with a saturated aqueous solution of sodium bicarbonate (50 mL), dried over anhydrous sodium sulfate, filtered, and the filtrate was concentrated under reduced pressure. The residue was purified by column chromatography (SiO₂, Petroleum ether/Ethyl acetate=3/1) to afford 7-nitro-4-(oxetan-3-yl)-3,4-dihydro-2H-benzo[b][1,4]oxazine (I-18-1) (0.426 g, 1.73 mmol, 62.4% yield) as a yellow solid.

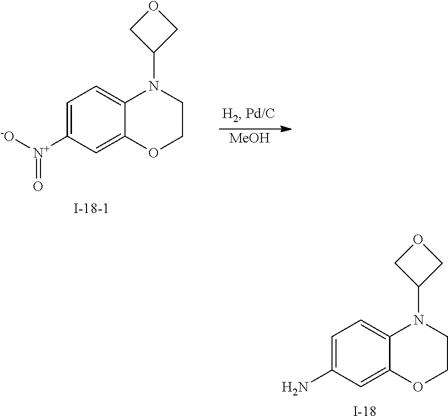

A solution I-18-1 (0.200 g, 0.847 mmol) in methanol (10 mL) was degassed and purged with nitrogen for three times. Then to the mixture was added Pd/C (0.020 g, 5% on charcoal, wet). The mixture was degassed and purged with hydrogen for three times. Then the mixture was stirred under hydrogen (15 psi) at 25° C. for 4 hours. The mixture was filtered and the filtrate was concentrated under reduced pressure. The residue was purified by prep-TLC (ethyl acetate:petroleum ether=2:1) to afford 4-(oxetan-3-yl)-3,4-dihydro-2H-benzo[b][1,4]oxazin-7-amine (I-18) (0.120 g, 0.500 mmol, 59.1% yield) as a black oil.

LCMS of I-18: m/z 207.2 [M+H]⁺

¹H-NMR of I-18: (CDCl₃, 400 MHz) δ 6.24 (d, J=2.4 Hz, 1H), 6.18 (dd, J=8.4, 2.4 Hz, 1H), 6.05 (d, J=8.4 Hz, 1H), 4.-5-4.87 (m, 1H), 4.-6-4.84 (m, 1H), 4.-1-4.56 (m, 2H), 4.-4-4.37 (m, 1H), 4.-7-4.32 (m, 2H), 3.-0-3.04 (m, 2H)

The following intermediate was synthesized in a similar manner as I-18 using the reactant indicated.

| | | LCMS | |
|---|---|---|---|
| Intermediate | Reactant | m/z | 1H-NMR |
| I-44 | 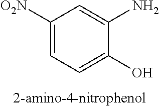 [SMILES: Nc1cc([N+](=O)[O-])ccc1O] | | (CDCl₃, 400 MHz) δ 6.63 (d, J = 8.4 Hz, 1H), 6.09 (dd, J = 8.4, 2.8 Hz, 1H), 5.66 (d, J = 2.4 Hz, 1H), 4.-0-4.78 (m, 4H), 4.-3-4.57 (m, 1H), 4.-2-4.25 (m, 2H), 3.-6-3.22 (m, 2H) |

### Synthesis of 3-fluoro-5-methyl-1H-indazole-7-sulfonyl chloride (I-19)

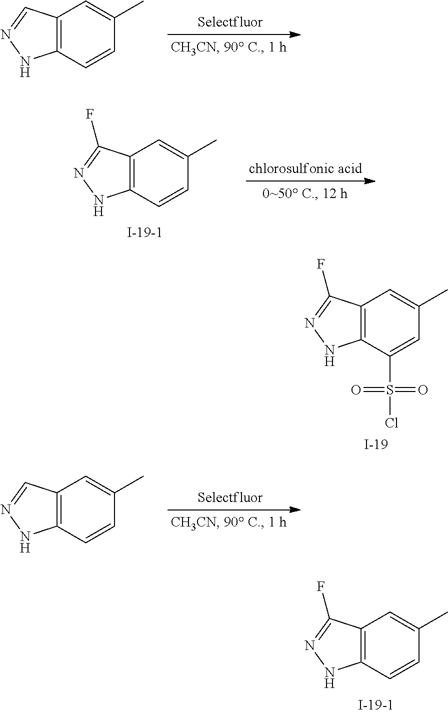

To a solution of 5-methyl-1H-indazole (4.00 g, 30.2 mmol) in CH₃CN (160 mL) was added Select Fluor (16.0 g, 45.4 mmol). The mixture was stirred at 90° C. for 1 hr under N₂. The reaction mixture was quenched by addition water (300 mL) and extracted with ethyl acetate (300 mL*3). The combined organic layers were dried over Na₂SO₄, filtered, and concentrated under reduced pressure to give a residue. The residue was purified by column chromatography (SiO₂, Petroleum ether/Ethyl acetate=4/1 to 3/1) to afford 3-fluoro-5-methyl-1H-indazole (I-19-1) (0.450 g, 2.49 mmol, 8.22% yield) as a yellow solid.

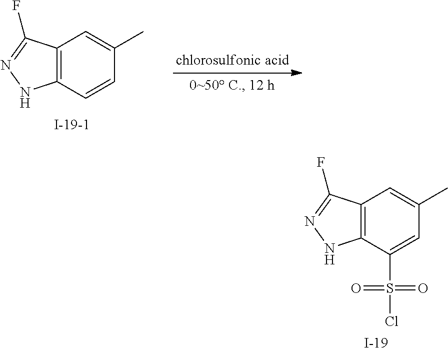

To chlorosulfonic acid (3.50 g, 30.0 mmol, 2.00 mL) was added I-19-1 (0.150 g, 0.998 mmol) at 0° C. The mixture was stirred at 20° C. for 0.5 hr and stirred at 50° C. for 11.5 hr. The reaction mixture was poured into ice/water (20 mL) and filtered. The filter cake was dissolved into ethyl acetate (20 mL), dried over Na₂SO₄, filtered and concentrated under reduced pressure to give a 3-fluoro-5-methyl-1H-indazole-7-sulfonyl chloride (I-19) (0.200 g, crude) as a yellow solid which was not further purified.

The following intermediates were synthesized in a similar manner as I-19.

| | | LCMS | |
|---|---|---|---|
| Intermediate | Reactant | m/z | 1H-NMR |
| I-40 | 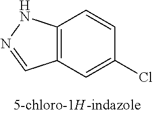 | | (DMSO-d₆, 400 MHz): δ 12.44 (br. s, 1H), 7.80 (d, J = 2.0 Hz, 1H), 7.49 (d, J = 2.0, 1H) |

### Synthesis of 3-fluoro-5-methyl-1H-indazole-7-sulfonyl chloride (I-23)

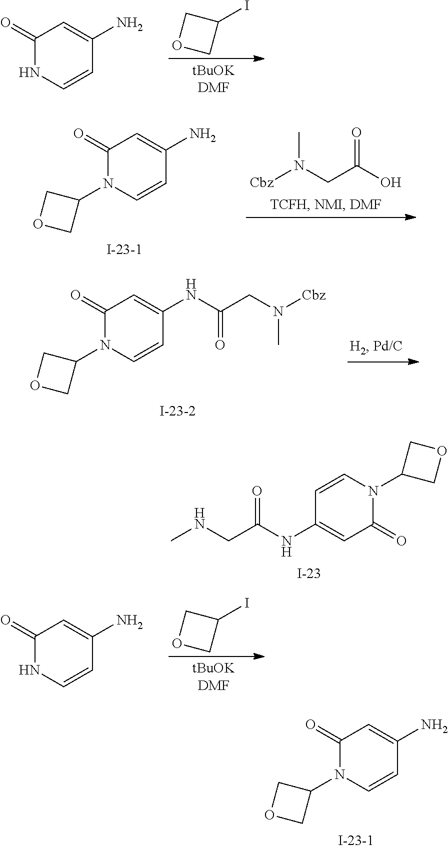

To a 4-aminopyridin-2(1H)-one (0.500 g, 4.54 mmol) and potassium tert-butoxide (0.750 g, 6.68 mmol) in N, N-dimethylformamide (5 mL) was added 3-iodooxetane (2.00 g, 10.9 mmol) at 0° C. Then the mixture was stirred at 80° C. for 36 hours. The mixture was diluted with water (50 mL) and lyophilized. The residue was purified by column chromatography (SiO₂, ethyl acetate/methanol=9/1) to afford 4-amino-1-(oxetan-3-yl) pyridin-2(1H)-one (I-23-1) (0.553 g, 3.33 mmol, 73.3% yield) as a white solid.

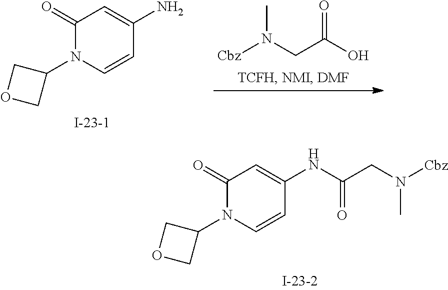 [SMILES: Nc1ccn(C2COC2)c(=O)c1 ; CN(CC(=O)O)C(=O)OCc1ccccc1 ; CN(CC(=O)Nc1ccn(C2COC2)c(=O)c1)C(=O)OCc1ccccc1]

To a solution of I-23-1 (0.553 g, 3.33 mmol) and N-((benzyloxy)carbonyl)-N-methylglycine (0.800 g, 3.58 mmol) in acetonitrile (5 mL) at 0° C. was added 1-methylimidazole (1.10 mL, 13.8 mmol) and N-(chloro(dimethylamino)methylene)-N-methylmethanaminium hexafluorophosphate (1.18 g, 4.21 mmol). The mixture was stirred at 40° C. for 12 hours then concentrated under reduced pressure. The residue was dissolved in a mixture of water (30 mL) and ethyl acetate (30 mL) then extracted with ethyl acetate (30 mL*5). The combined organic phase was dried over anhydrous sodium sulfate, filtered, and the filtrate was concentrated under reduced pressure. The crude product was purified by reversed-phase HPLC (0.1% NH₃·H₂O/MeCN condition) to afford benzyl methyl(2-((1-(oxetan-3-yl)-2-oxo-1,2-dihydropyridin-4-yl)amino)-2-oxoethyl) carbamate (I-23-2) (0.070 g, 0.176 mmol, 5.29% yield) as a yellow solid.

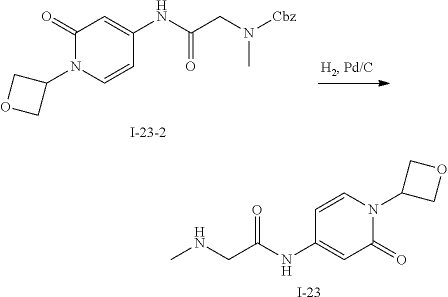

A solution of benzyl methyl I-23-2 (0.070 g, 0.188 mol) in propan-2-ol (20 mL) was degassed and purged with nitrogen for three times. Then Pd/C (10 mg, 5% on charcoal, wet) was added. The mixture was degassed and purged with hydrogen for three times. Then the mixture was stirred at 25° C. for 10 hours under hydrogen atmosphere (15 psi). The mixture was filtered, and the filtrate was concentrated under reduced pressure to afford 2-(methylamino)-N-(1-(oxetan-3-yl)-2-oxo-1, 2-dihydropyridin-4-yl) acetamide (I-23) (0.040 g, 0.150 mmol, 79.6% yield) as yellow oil.

LCMS of I-23: m/z 238.1 [M+H]⁺

¹H-NMR of I-23: (CD₃OD, 400 MHz): δ 7.76 (d, J=7.6 Hz, 1H), 6.98 (d, J=2.4 Hz, 1H), 6.68 (dd, J=7.6, 2.4 Hz, 1H), 5.-2-5.52 (m, 1H), 5.01 (t, J=7.6 Hz, 2H), 4.-4-4.81 (m, 2H), 3.81 (s, 2H), 2.67 (s, 3H)

The following intermediate was synthesized in a similar manner as I-23 using the reactant indicated.

| | | LCMS | |
|---|---|---|---|
| Intermediate | Reactant | m/z | 1H-NMR |
| I-45 | I-44 | 278.1 | (CDCl3, 400 MHz) δ 7.05 (d, J = 2.4 |
| | | | Hz, 1H), 6.73 (d, J = 8.4 Hz, 1H), 6.58 |
| | | | (dd, J = 8.4, 2.4 Hz, 1H), 4.92 (t, J = |
| | | | 7.2 Hz, 2H), 4.82 (t, J = 7.2 Hz, 2H), |
| | | | 4.−9-4.60 (m, 1H), 4.−7-4.32 (m, |
| | | | 2H), 3.32 (s, 2H), 3.−6-3.23 (m, 2H), |
| | | | 2.49 (s, 3H) |

### Synthesis of 2-(methylamino)-N-(2-oxo-1-(tetrahydro-2H-pyran-4-yl)-1,2-dihydropyridin-4-yl)acetamide (I-24)

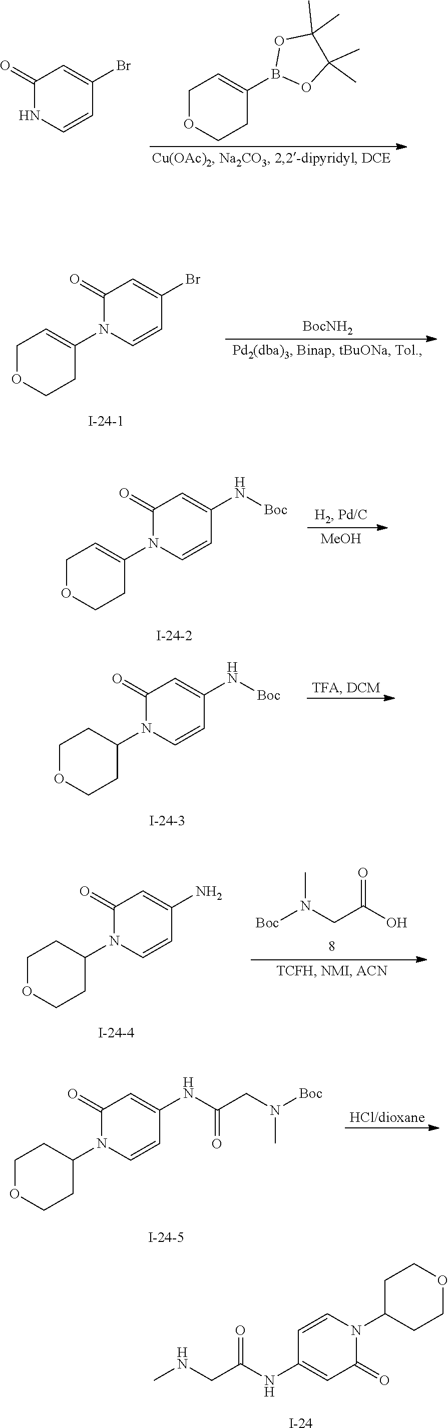

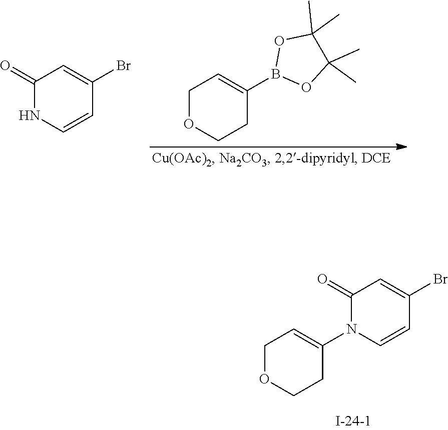

To a mixture of 4-bromopyridin-2(1H)-one (2.00 g, 11.5 mmol) and 2-(3,6-dihydro-2H-pyran-4-yl)-4,4,5,5-tetramethyl-1,3,2-dioxaborolane (4.66 g, 22.2 mmol) in 1, 2-dichloroethane (50 mL) was added sodium carbonate (3.65 g, 34.5 mmol), copper (II) acetate (2.51 g, 13.8 mmol) and 2-(2-pyridyl)pyridine (1.97 g, 12.6 mmol) at 25° C. The mixture was stirred at 70° C. for 15 hours under oxygen (15 psi). The reaction mixture was poured into water (250 mL), and then extracted with dichloromethane (80 mL*3). The combined organic layers were washed with brine (250 mL), dried over anhydrous sodium sulfate, filtered and the filtrate was concentrated under reduced pressure. The residue was purified by column chromatography (SiO₂, Petroleum ether:Ethyl acetate=20:1˜1:1) to afford 4-bromo-1-(3,6-dihydro-2H-pyran-4-90ydroxyadin-2(1H)-one (I-24-1) (1.30 g, 4.53 mmol, 39.4% yield) as a yellow oil.

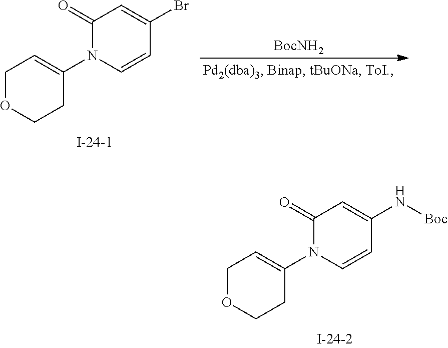

To a solution of I-24-1 (1.30 g, 4.53 mmol) in toluene (15 mL) was added tert-butyl carbamate (0.636 g, 5.43 mmol), Pd₂(dba)₃ (0.207 mg, 0.226 mmol), 2, 2-bis(diphenylphosphino)-1, 1-binaphthalene (0.282 g, 0.453 mmol) and sodium tert-butoxide (0.870 g, 9.05 mmol) at 25° C. and the mixture was stirred at 90° C. for 3 hours. The reaction mixture was poured into water (150 mL), filtered and the solid was triturated with ethyl acetate (30 mL) to afford tert-butyl (1-(3,6-dihydro-2H-pyran-4-yl)-2-oxo-1,2-dihydropyridin-4-yl)carbamate (I-24-2) (1.00 g, 3.36 mmol, 74.17% yield) as a white solid.

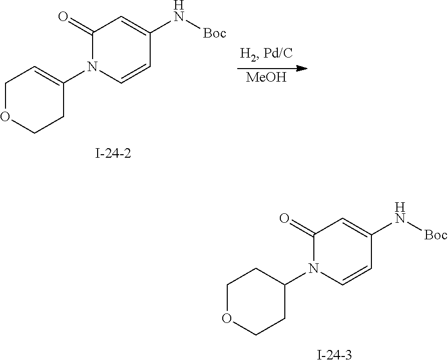 [SMILES: "MeOH" ; CC(C)(C)OC(=O)Nc1ccn(C2=CCOCC2)c(=O)c1 ; CC(C)(C)OC(=O)Nc1ccn(C2CCOCC2)c(=O)c1]

To a solution of I-24-2 (1.00 g, 3.36 mmol) in methanol (20 mL) was added Pd/C (0.100 g, 0.048 mmol, 10% on wet charcoal) at 25° C. and the mixture was stirred at 25° C. for 1 hour under hydrogen (15 psi). The mixture was filtered and the filtrate was concentrated under reduced pressure to give a residue. The residue was purified by column (SiO₂, petroleum ether:ethyl acetate=1:1˜dichloromethane:methanol=50:1) to afford tert-butyl (2-oxo-1-(tetrahydro-2H-pyran-4-yl)-1,2-dihydropyridin-4-yl)carbamate (I-24-3) (0.620 g, 2.02 mmol, 60.3% yield) as a white solid.

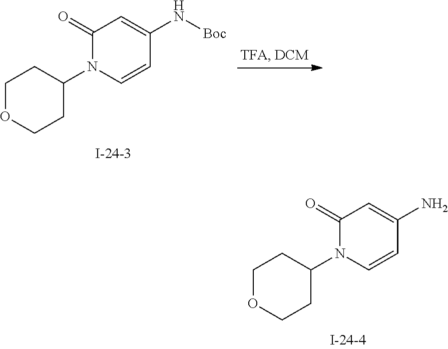 [SMILES: CC(C)(C)OC(=O)Nc1ccn(C2CCOCC2)c(=O)c1 ; Nc1ccn(C2CCOCC2)c(=O)c1]

To a solution of I-24-3 (0.500 g, 1.63 mmol) in dichloromethane (6 mL) was added trifluoroacetic acid (2 mL, 27.0 mmol) and the mixture was stirred at 25° C. for 1 hour. The mixture was concentrated under reduced pressure to give a residue. Then the residue was dissolved in water (0.5 mL) and adjusted to pH=9 with a sodium hydroxide solution. The crude product was purified by reversed-phase HPLC (0.1% NH₃H₂O condition) to provide 4-amino-1-(tetrahydro-2H-pyran-4-y91ydroxyadin-2(1H)-one (I-24-4) (0.100 g, 0.515 mmol, 31.6% yield) as a white solid.

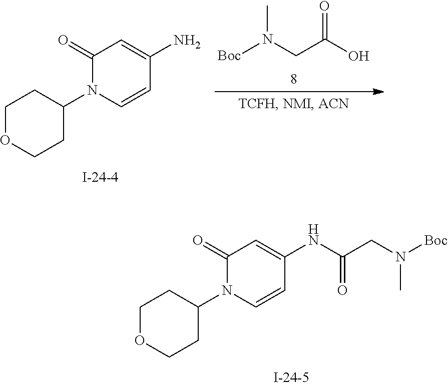 [SMILES: Nc1ccn(C2CCOCC2)c(=O)c1 ; CN(CC(=O)O)C(=O)OC(C)(C)C ; CN(CC(=O)Nc1ccn(C2CCOCC2)c(=O)c1)C(=O)OC(C)(C)C]

To a solution of I-24-4 (0.080 g, 0.412 mmol) and 2-((tert-butoxycarbonyl)(methyl)amino)acetic acid (0.156 g, 0.824 mmol) in acetonitrile (1 mL) was added 1-methylimidazole (0.271 g, 3.30 mmol) and [chloro(dimethylamino)methylene]-dimethyl-ammonium; hexafluorophosphate (0.347 g, 1.24 mmol) at 0° C. and the mixture was stirred at 40° C. for 12 hours. The mixture was concentrated under reduced pressure and the residue was purified by reversed-phase HPLC (0.1% NH₃H₂O condition) to afford tert-butyl methyl(2-oxo-2-((2-oxo-1-(tetrahydro-2H-pyran-4-yl)-1,2-dihydropyridin-4-yl)amino)ethyl)carbamate (I-24-5) (0.020 g, 0.053 mmol, 12.9% yield) as a yellow solid.

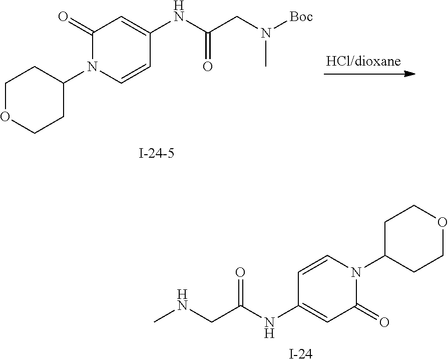

To a solution of I-24-5 (0.020 g, 0.053 mmol) in dioxane (0.5 mL) was added a solution of HCl in dioxane (4 M, 0.5 mL) at 25° C. and the mixture was stirred at 25° C. for 1 hour. The mixture was concentrated under reduced pressure to afford 2-(methylamino)-N-(2-oxo-1-(tetrahydro-2H-pyran-4-yl)-1,2-dihydropyridin-4-yl)acetamide (I-24), as the hydrochloric salt (0.030 g, crude), as a white solid which was used without further purification.

### Synthesis of 2-(7-amino-2,3-dihydro-4H-benzo[b][1,4]oxazin-4-yl)-2-methylpropan-1-ol (I-25)

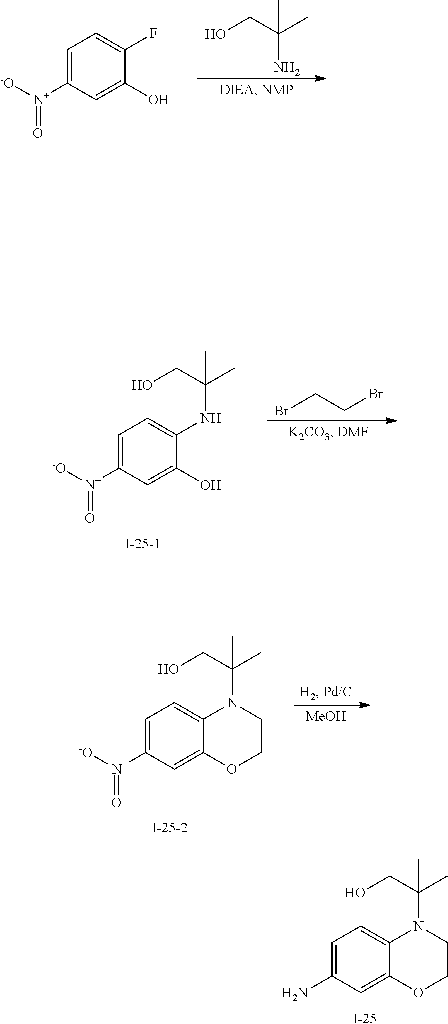

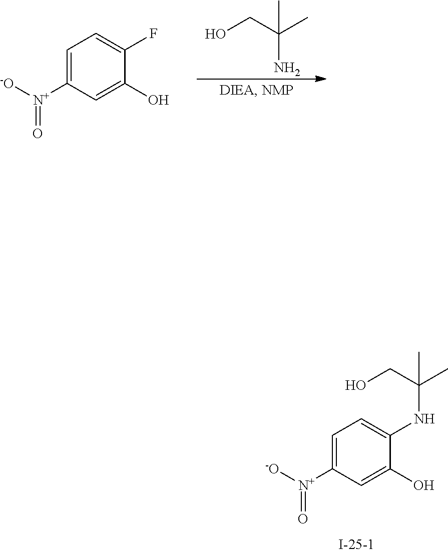

A mixture of 2-fluoro-5-nitrophenol (1.00 g, 6.37 mmol), 2-amino-2-methylpropan-1-ol (0.999 g, 11.2 mmol) and N,N-diisopropylethylamine (2.97 g, 22.98 mmol) were taken up into a microwave tube in N-methyl pyrrolidone (10 mL). The sealed tube was heated at 180° C. for 2 hours under microwave irradiation then poured into water (50 mL) and extracted with ethyl acetate (100 mL*3). The combined organic layers were washed with brine (100 mL*3), dried over anhydrous sodium sulfate, filtered, and the filtrate was concentrated under reduced pressure. The residue was purified by prep-HPLC (column: Phenomenex luna C18 150*40 mm*15 um; mobile phase: [water (0.05% HCl)-ACN]; B %: 15%-45%, 10 min) to afford 2-((1-hydroxy-2-methylpropan-2-yl)amino)-5-nitrophenol (I-25-1) (0.400 g, 0.972 mmol, 7.64% yield) as a yellow solid.

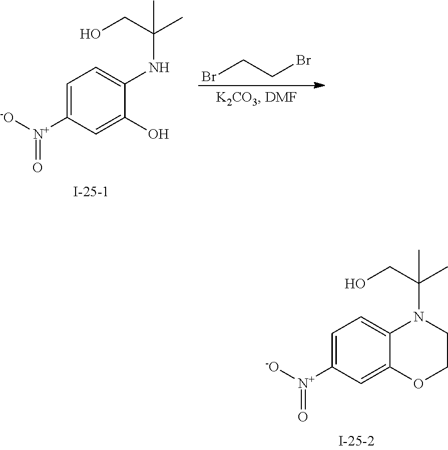

To a solution of I-25-1 (6.00 g, 26.5 mmol) in N,N-dimethylformamide (5 mL) was added 1,2-dibromoethane (4.98 g, 26.5 mmol) and potassium carbonate (11.0 g, 79.6 mmol) at 25° C. under nitrogen atmosphere. The mixture was heated to 80° C. for 12 hours then poured into water (50 mL) and extracted with ethyl acetate (30 mL*3). The combined organic layers were washed with brine (100 mL*3), dried over anhydrous sodium sulfate, filtered, and the filtrate was concentrated under reduced pressure. The residue was purified by column chromatography (SiO₂, petroleum ether/ethyl acetate=1/0 to 3/1) to afford 2-methyl-2-(7-nitro-2H-benzo[b][1,4]oxazin-4(3H)-yl)propan-1-ol (I-25-2) (0.280 g, 0.901 mmol, 3.40% yield) as a yellow solid.

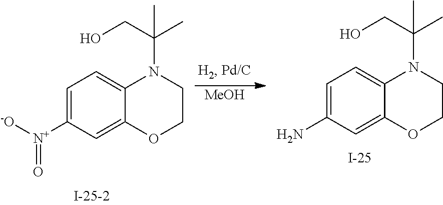

To a solution of I-25-2 (0.150 g, 0.483 mmol) in methanol (2 mL) was added Pd/C (0.015 g, 10% purity on charcoal, wet) under nitrogen. The suspension was degassed under vacuum and purged with hydrogen (15 psi) several times then stirred under hydrogen (15 psi) at 25° C. for 2 hours. The mixture was filtered and filtrate was concentrated in vacuum to afford 2-(7-amino-2H-benzo[b][1,4]oxazin-4(3H)-yl)-2-methylpropan-1-ol (I-25) (0.110 g, crude) as black brown oil. No further purification was performed.

¹H-NMR of I-25: (DMSO-d6, 400 MHz): δ 6.-5-6.63 (m, 1H), 6.-0-5.97 (m, 2H), 4.58 (br. s, 2H), 4.54 (br. t, J=5.2 Hz, 1H), 4.10 (t, J=4.8 Hz, 2H), 3.15 (t, J=4.8 Hz, 2H), 1.08 (s, 6H).

### Synthesis of 5-methyl-1H-indazole-7-sulfonyl chloride (I-31)

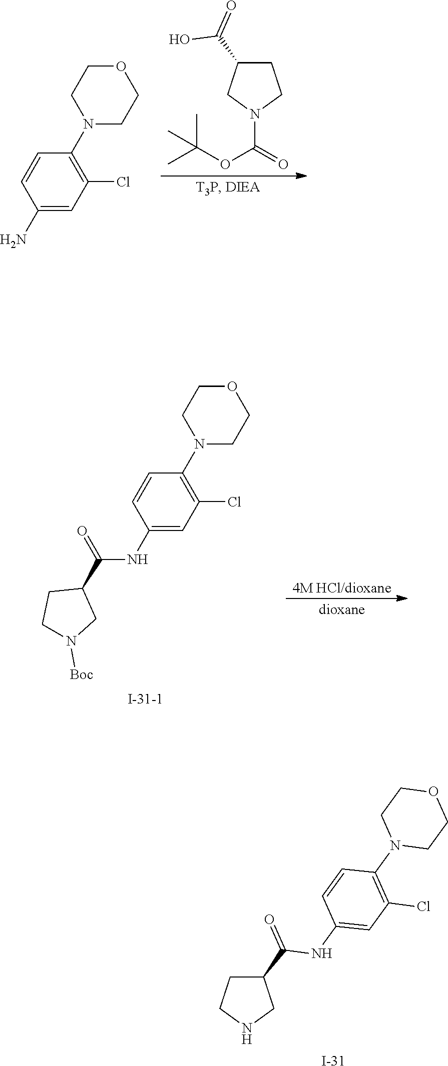

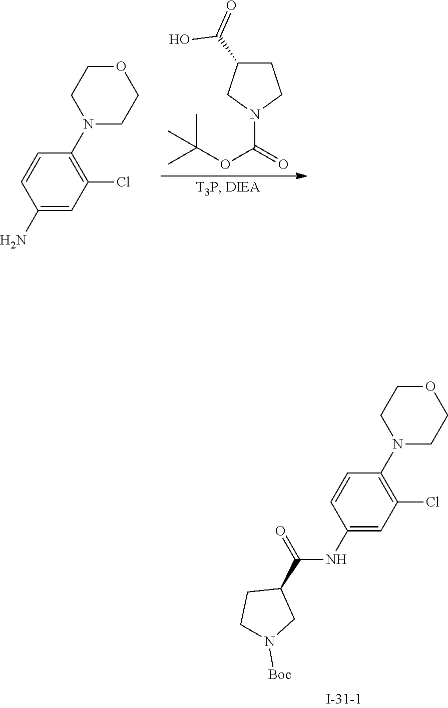

To a solution I(R)-1-(tert-butoxycarbonyl)pyrrolidine-3-carboxylic acid (0.202 g, 0.940 mmol) and 3-chloro-4-morpholinoaniline (0.200 g, 0.940 mmol) in N, N-dimethylformamide (2 mL) at 0° C. was added N, N-diisopropylethylamine (0.660 mL, 3.79 mmol) and T₃P (0.840 mL, 1.41 mmol, with 50% solution in ethyl acetate) drop wise. Then the mixture was warmed to 25° C. slowly within 1 hour. The mixture was quenched by water (10 mL), diluted with a saturated aqueous solution of sodium bicarbonate (10 mL), and extracted with ethyl acetate (10 mL*3). The combined organic phase was washed with 1N HCl and adjusted to pH=5, followed by brine (20 mL), dried over anhydrous sodium sulfate, filtered, and the filtrate was concentrated under reduced pressure to afford tert-Iyl (R)-3-((3-chloro-4-morpholinophenyl)carbamoyl)pyrrolidine-1-carboxylate (I-31-1) (0.330 g, 0.780 mmol, 82.9% yield) as brown oil.

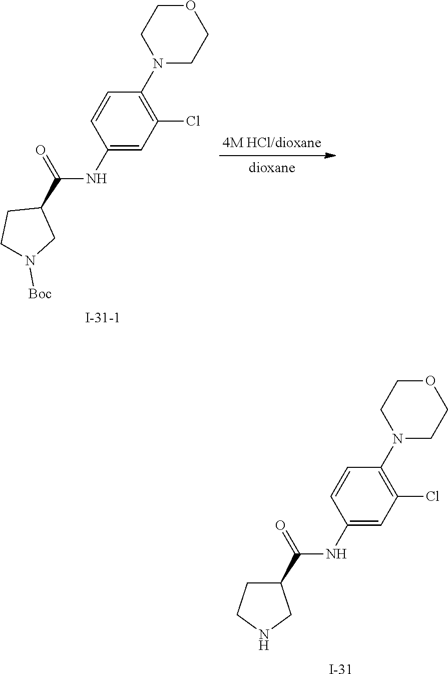

To a solution of I-31-1 (0.165 g, 0.385 mmol) in dioxane (2 mL) at 0° C. was added a solution of HCl in dioxane (4 M, 1.00 mL) drop wise. Then the mixture was stirred at 25° C. for 3 hours. The mixture was concentrated under reduced pressure tIfford (R)—N-(3-chloro-4-morpholinophenyl)pyrrolidine-3-carboxamide (I-31) as the hydrochloric salt (0.130 g, 0.370 mmol, 95.9% yield) as a pink solid.

LCMS of I-31: m/z 310.3 [M+H]⁺

¹H-NMR of I-31: (DMSO-d6, 400 MHz) δ 10.46 (br. s, 1H), 9.30 (br. s, 1H), 9.08 (br. s, 1H), 7.82 (d, J=2.4 Hz, 1H), 7.47 (dd, J=8.8, 2.4 Hz, 1H), 7.14 (d, J=8.8 Hz-1H), 3.74-3.71 (m-4H), 3.41-3.19 (m-5H), 2.94-2.87 (m-4H), 2.29-2.19 (m-1H), 2.07-2.00 (m, 1H)

The following intermediates were synthesized in a similar manner as I-31 using the reactant indicated.

| | | LCMS | |
|---|---|---|---|
| Intermediate | Reactant | m/z | 1H-NMR |
| I-32 | 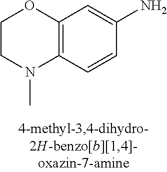 | 262.2 | (CDCl₃, 400 MH-): δ 10.67-10.57 (br. m, 4H), 8.56(s, 1H), 7.22-7.00 (m, 1H), 4.23(s, 3H), 4.16(s-2H), 3.59-3.12 (m-7H), 2.34-2.02(m, 2H) |
| I-33 | I-15 | 332.4 | (CD₃OD, 400 MHz): δ 7.08(d, |
| | | | J = 2.0 Hz-1H), 7.00-6.98(m, 1H), |
| | | | 6.88(d, J = 9.2 Hz,-1H), 4.25- |
| | | | 4.23(m-2H), 4.15-4.11(m-3H), |
| | | | 3.97-3.73(m-1H), 3.63-3.43(m- |
| | | | 8H), 2.42-2.34(m-1H), 2.27- |
| | | | 2.23(m-1H), 1.86-1.80(m-2H), |
| | | | 1.75-1.71(m, 2H) |

The following intermediate was synthesized in a similar manner as I-31 replacing (R)-1-(tert-butoxycarbonyl)pyrrolidine-3-carboxylic acid with (S)-1-(tert-butoxycarbonyl)azetidine-2-carboxylic acid

| | | LCMS | |
|---|---|---|---|
| Intermediate | Reactant | m/z | 1H-NMR |
| I-49 | 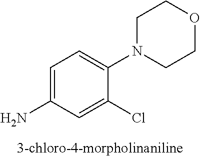 [SMILES: Nc1ccc(N2CCOCC2)c(Cl)c1] | 296.1 | (DMSO-d₆, 400 MHz) δ 10.99 (br, s, 1H), 9.68 (br, s, 1H), 8.90 (br, s, 1H), 8.58 (br, s, 1H), 7.82 (d, J = 2.4 Hz, 1H), 7.53 (dd, J₁ = 2.4 Hz, J2 = 8.8 Hz, 1H), 7.18 (d, J = 8.8 Hz-1H), 5.18-5.04 (m-1H), 4.04- 3.90 (m, 1H), 3.77 (s-1H), 3.75- 3.72 (m-4H), 2.95-2.91 (m-4H), 2.74-2.66 (m-1H), 2.60-2.52 (m, 1H) |
| I-52 | I-51 | 298.1 | (DMSO-d₆, 400 MHz) δ 10.39 (br, |
| | | | s, 1H), 7.82 (d, J = 2.4 Hz, 1H), |
| | | | 7.47 (dd, J₁ = 2.4 Hz, J₂ = 8.8 Hz- |
| | | | 1H), 7.22-7.14 (m, 1H), 4.86 (t, |
| | | | J = 8.4 Hz, 1H), 4.19 (t, J = 5.2 |
| | | | Hz, 2H), 3.90 (q, J = 8.8 Hz, 1H), |
| | | | 3.68 (dt, J₁ = 5.6 Hz, J₂ = 9.2 Hz- |
| | | | 1H), 2.97-2.86 (m− 2H), 2.72- |
| | | | 2.60 (m-1H), 2.47-2.39 (m, 7H) |
| I-54 | I-48 | 278.0 | (DMSO-d₆, 400 MHz) δ 10.41 (br, |
| | | | s, 1H), 7.44 (dd, J₁ = 2.4 Hz, J₂ = |
| | | | 8.8 Hz, 1H), 7.38 (d, J = 2.0 Hz, |
| | | | 1H), 6.97 (d, J =8.8 Hz-1H), 5.09- |
| | | | 5.04 (m-1H), 4.30-4.27 (m, 2H), |
| | | | 4.21 (s-1H), 4.05-3.98 (m-1H), |
| | | | 3.85-3.76 (m-2H), 3.53-3.51 (m, |
| | | | 2H), 2.88 (s, 6H), 2.18 (s, 3H) |

### Synthesis of (R)—N-(4-(oxetan-3-yl)-3,4-dihydro-2H-benzo[b][1,4]oxazin-7-yl)pyrrolidine-3-carboxamide (I-34)

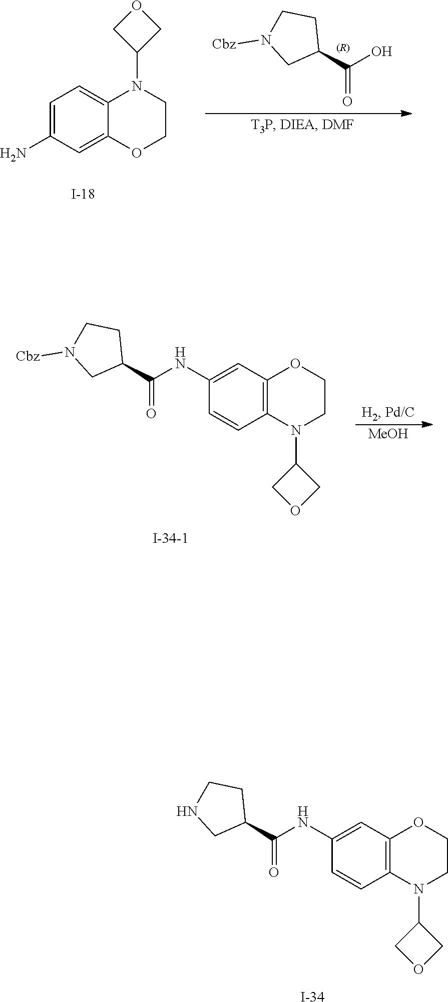

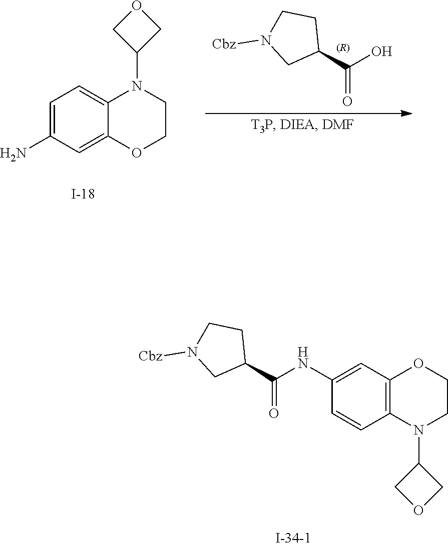

To a solution of I-18 (0.150 g, 0.536 mmol) and (R)-1-((benzyloxy)carbonyl)pyrrolidine-3-carboxylic acid (0.134 g, 0.536 mmol) in N,N-dimethylformamide (1 mL) was added diisopropylethylamine (0.468 mL, 2.68 mmol) and a solution of T₃P (50% solution in ethyl acetate) (0.479 mL, 0.805 mmol) drop wise at 0° C. and stirred for 1 hour. The mixture was added into ice-water (10 mL), and extracted with ethyl acetate (20 mL*3). The combined organic phase was washed with brine (10 mL*2), dried over anhydrous sodium sulfate, filtered, and concentrated under reduced presIe to afford (R)-benzyl 3-((4-(oxetan-3-yl)-3,4-dihydro-2H-benzo[b][1,4]oxazin-7-yl)carbamoyl)pyrrolidine-1-carboxylate (I-34-1) (0.230 g, 0.494 mmol, 92.1% yield) as a black oil.

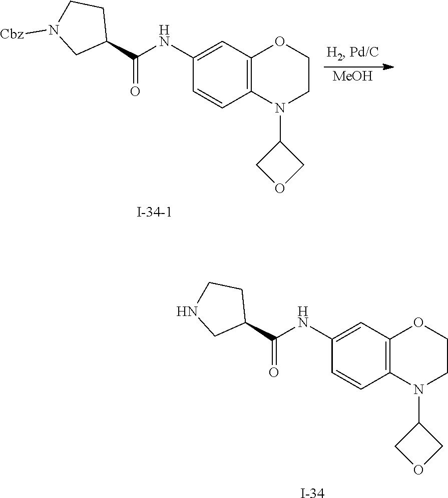

A solution of I-34-1 (0.100 g, 0.215 mmol) in methanol (3 mL) was degassed and purged with nitrogen for three times. Then to the mixture was added Pd/C in mineral oil with 10% purity (0.020 g). The mixture was degassed and purged with hydrogen for three times. Then the mixture was stirred under hydrogen (15 psi) at 25° C. for 2 hours. The mixture was filtered and the filtrate cake was washed with methanol (10 mL*2). The combined filtrate was concentrated under reduced pressure to afford (R)—N-(4-(oxetan-3-yl)-3,4-dihydro-2H-benzo[b][1,4]oxazin-7-yl)pyrrolidine-3-carboxamide (I-34) (0.090 g) as a yellow oil.

¹H-NMR of I-34: (CDCl₃, 400 MHz): δ 8.-8 (br. s, 1H), 6.97-6.94 (m, 2H), 6.15 (d, J=8.0 Hz, 1H), 4.87-4.78 (m, 5H), 4.52 (t, J=6.8 Hz, 1H), 4.36 (t, J=4.4 Hz, 2H), 3.28 (dd, J=10.-, 3.6 Hz, 1H), 3.21-3.19 (m, 1H), 3.17 (t, J-=4.4 Hz, 2H), 2.86-2.83 (m, 2H), 2.13-2.11 (m, 1H), 2.05-2.02 (m, 1H).

The following intermediate was synthesized in a similar manner as I-34 replacing (R)-1-((benzyloxy)carbonyl)pyrrolidine-3-carboxylic acid with (S)-1-((benzyloxy)carbonyl)azetidine-2-carboxylic acid

| Inter- | | LCMS | |
|---|---|---|---|
| mediate | Reactant | m/z | 1H-NMR |
| I-46 | I-18 | 290.0 | (CDCl3, 400 MHz) δ 9.32 (br. s, 1H), |
| | | | 7.12 (d, J = 2.4 Hz, 1H), 7.08 (dd, J1 = |
| | | | 2.4 Hz, J2 = 8.6 Hz, 1H), 6.19 (d, J −= 8.6 |
| | | | Hz, 1H), 4.89-4.79 (m, 4H), 4.59-4.50 |
| | | | (m, 1H), 4.44-4.39 (m, 1H), 4.39-4.34 |
| | | | (m, 2H), 3.88-3.76 (m, 1H), 3.41-3.30 |
| | | | (m, 1H), 3.22-3.15 (m, 2H), 2.75-2.64 |
| | | | (m, 1H), 2.50-2.36 (m, 1H) |
| I-55 | I-44 | | (CD₃OD, 400 MHZ) δ 6.81 (d, J −= 2.0 |
| | | | Hz, 1H), 6.78-6.75 (m, 1H), 6.68-6.66 |
| | | | (m, 1H), 4.54 (q, J = 8.0 Hz, 1H), 4.39 |
| | | | (dd, J₁ = 7.2 Hz, J₂ −= 8.0 Hz, 1H), 4.34- |
| | | | 4.30 (m, 2H), 3.71 (q, J = 8.0 Hz, 1H), |
| | | | 3.47 (dd, J₁ = 5.2 Hz, J₂ −= 7.2 Hz, 1H), |
| | | | 3.22-3.16 (m, 2H), 2.69-2.68 (m, 1H), |
| | | | 2.43-2.40 (m, 1H) |

### Synthesis of N-ethyl-N-((5-methyl-1H-indazol-7-yl)sulfonyl)glycine (I-38)

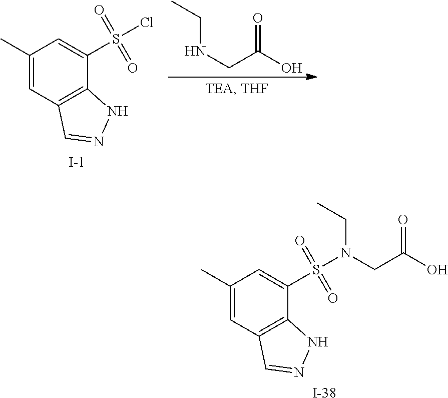

To a solution of ethylglycine (0.090 g, 0.873 mmol) in tetrahydrofuran (2 mL) at 0° C. was added triethylamine (0.500 mL, 3.59 mmol) and I-1 (0.200 g, 0.867 mmol). Then the mixture was stirred at 25° C. for 2 hours then concentrated under reduced pressure. The crude product was purified by reversed-phase flash (0.1% TFA/MeCN condition) to afford N-ethyl-N-((5-methyl-1H-indazol-7-yl) sulfonyl)glycine (I-38) (0.070 g, 27.2% yield) as a white solid.

LCMS of I-38: RT=0.689 min, m/z 298.0 [M+H]⁺

1H-NMR of I-38: (DMSO-d6, 400 MHz) δ 13.17 (br. s, 1H), 12.65 (s, 1H), 8.16 (s, 1H), 7.86 (s, 1H), 7.65 (d, J=1.2 Hz, 1H), 4.16 (s, 2H), 3.25 (q, J=7.2 Hz, 2H), 2.46 (s, 3H), 0.93 (t, J=7.2 Hz, 3H)

### Synthesis of 1-(7-amino-2,3-dihydro-4H-benzo[b][1,4]oxazin-4-yl)-2-methylpropan-2-ol (I-39)

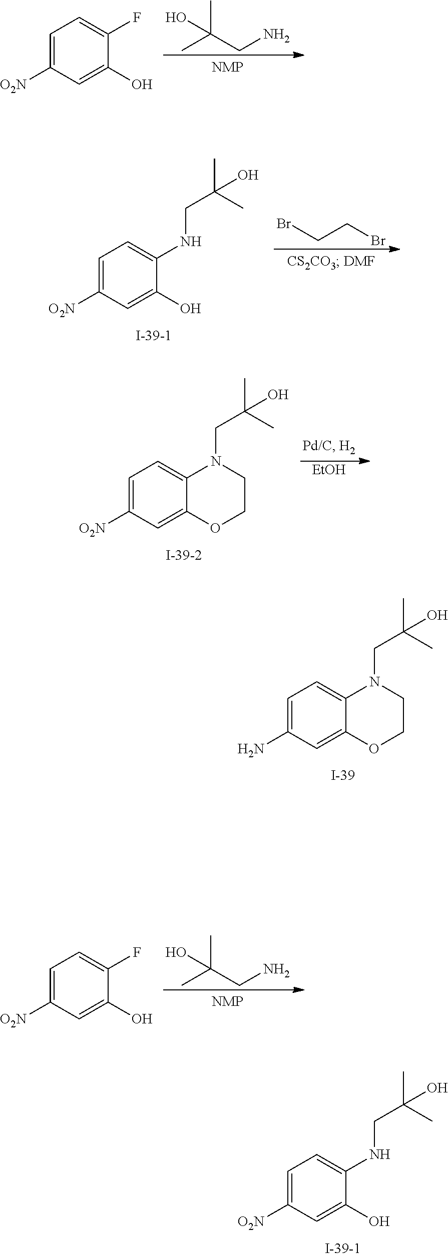

To a solution of 2-fluoro-5-nitrophenol (1.00 g, 6.37 mmol) and 1-amino-2-methylpropan-2-ol (2.30 g, 25.8 mmol) in 1-methylpyrrolidin-2-one (10 mL) was added diisopropylethylamine (3.5 mL, 20.1 mmol). The mixture was stirred at 180° C. for 2 hours under microwave condition. The mixture was poured into water (300 mL) and extracted with ethyl acetate (100 mL*6). The combined organic layers were washed with brine (50 mL), dried over anhydrous sodium sulfate, filtered, and the filtrate was concentrated under reduced pressure. The residue was purified by column chromatography (SiO₂, petroleum ether/ethyl acetate=1/1˜0/1) to afford 2-((2-hydroxy-2-methylpropyl)amino)-5-nitrophenol (I-39-1) (1.40 g, 97% yield) as yellow oil.

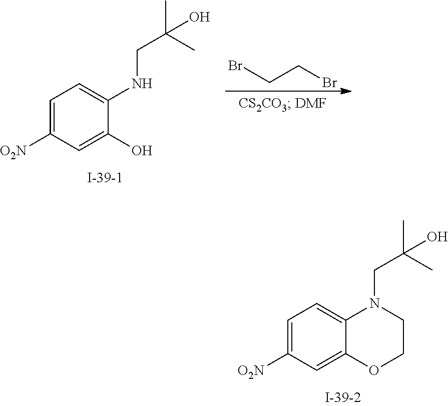

To a solution of I-39-1 (0.500 g, 2.21 mmol) in dimethyl formamide (20 mL) was added cesium carbonate (2.20 g, 6.75 mmol) and 1,2-dibromoethane (0.2 mL, 2.65 mmol). The mixture was stirred at 25° C. for 3 hours under nitrogen atmosphere. The mixture was poured into water (100 mL) and extracted with ethyl acetate (30 mL*6). The combined organic layers were washed with brine (20 mL), dried over anhydrous sodium sulfate, filtered, and the filtrate was concentrated under reduced pressure. The crude product was purified by reversed-phase HPLC (0.1% TFA condition) to afford 2-methyl-1-(7-nitro-2, 3-dihydro-4H-benzo[b][1,4]oxazin-4-yl)propan-2-ol (I-39-2) (0.120 g, 21% yield) as yellow oil.

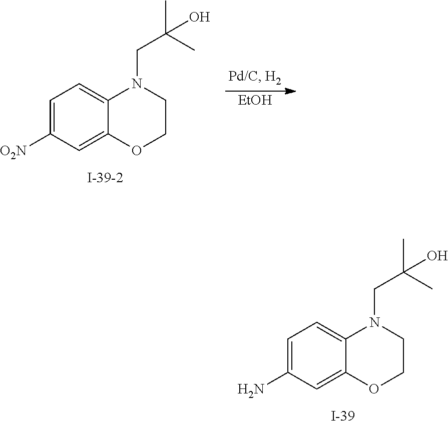

A solution of I-39-2 (0.060 g, 0.238 mmol) in ethyl alcohol (2 mL) was degassed and purged with nitrogen for three times. Pd/C (0.010 g, 10% on charcoal, wet) was added and the mixture was degassed and purged with hydrogen (15 psi) for three times. The mixture was stirred at 25° C. for 1 hour under hydrogen atmosphere. The mixture was filtered, and the filtrate was concentrated under reduced pressure to afford 1-(7-amino-2, 3-dihydro-4H-benzo[b][1,4]oxazin-4-yl)-2-methylpropan-2-ol (I-39) (0.040 g, 71% yield) as black brown oil. No further purification was performed.

LCMS of I-39: RT=0.459 min, m/z 223.5 [M+H]⁺

¹H-NMR of I-39: (CD-13, 400 MHz) δ 6.81-6.70 (m, 1H), 6.30-6.19 (m, 2H), 4.24-4.17 (m, 2H), 3.40-3.31 (m, 2H), 3.07 (s, 2H), 1.29 (s, 6H)

### Synthesis of 5-(difluoromethyl)-1H-indazole-7-sulfonyl chloride (Intermediate I-43)

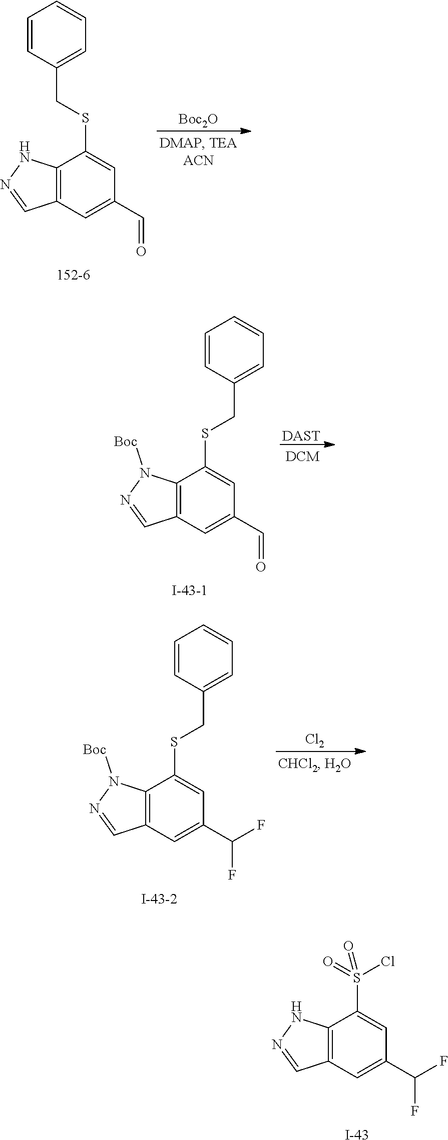

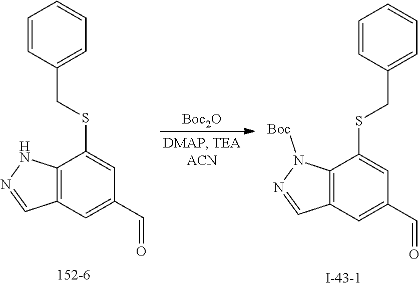 [SMILES: "Boc2O" ; O=Cc1cc(SCc2ccccc2)c2[nH]ncc2c1 ; CC(C)(C)OC(=O)n1ncc2cc(C=O)cc(SCc3ccccc3)c21]

To a solution of 152-6 (10.0 g, 37.3 mmol) in acetonitrile (100 mL) was added triethylamine (10.4 mL, 74.5 mmol) and DMAP (0.455 g, 3.73 mmol) follo102ydroxyaminert-butyl dicarbonate (15.4 mL, 67.1 mmol) drop wise. The mixture was stirred at 25° C. for 4 hours, then diluted with water (100 mL) and extracted with ethyl acetate (100 mL*2). The combined organic layers were washed with brine (100 mL), dried over anhydrous sodium sulfate, filtered, and the filtrate was concentrated under reduced pressure. The residue was purified by column chromatography (SiO₂, petroleum ether/ethyl acetate=20/1 to 8/1) to afford tert-butyl 7-(benzylthio)-5-formyl-1H-indazole-1-carboxylate (I-43-1) (5.00 g, 33.5% yield) as a yellow solid.

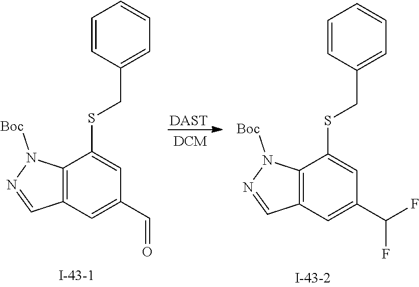

To a solution of I-43-1 (4.0 g, 10.9 mmol) in dichloromethane (40 mL) was added DAST (11.5 mL, 86.8 mmol) at 0° C. The mixture was stirred at 25° C. for 12 hours then quenched with a saturated aqueous solution of sodium bicarbonate (500 mL) and extracted with dichloromethane (100 mL*2). The combined organic layers were washed with brine (100 mL), dried over anhydrous sodium sulfate, filtered, and the filtrate was concentrated under reduced pressure. The residue was purified by column chromatography (SiO₂, petroleum ether/ethyl acetate=20/1˜10/1) to afford tert-butyl 7-(benzylthio)-5-(difluoromethyl)-1H-indazole-1-carboxylate (I-43-2) (3.30 g, 75.1% yield) as a yellow solid.

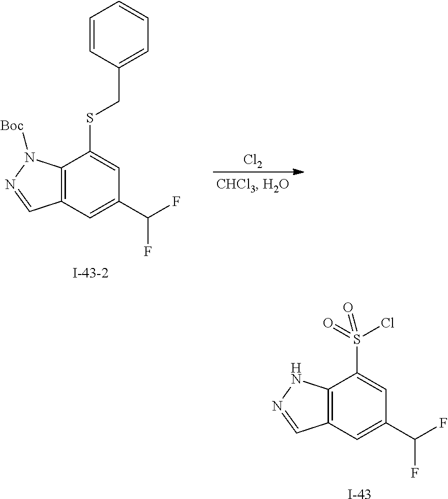

To a solution of I-43-2 (0.100 g, 0.256 mmol) in and mixture of chloroform (6 mL) and water (3 mL), at 0° C., was bubbled chlorine gas for 10 minutes, then the excess chlorine was purged with nitrogen. Cold water (100 mL) was added and the mixture was extracted with dichloromethane (100 mL*2). The combined organic phase was dried over anhydrous sodium sulfate, filtered and the filtrate was concentrated under reduced pressure to afford tert-butyl 7-(chlorosulfonyl)-5-(difluoromethyl)-1H-indazole-1-carboxylate (I43) (0.068 g, crude) as yellow solid. No further purification was performed.

LCMS of I-43: RT=1.013 min, m/z 267.0 [M+H]⁺

### Synthesis of N-((2-cyano-5-methyl-1H-indol-7-yl)sulfonyl)-N-methylglycine (Intermediate I-47)

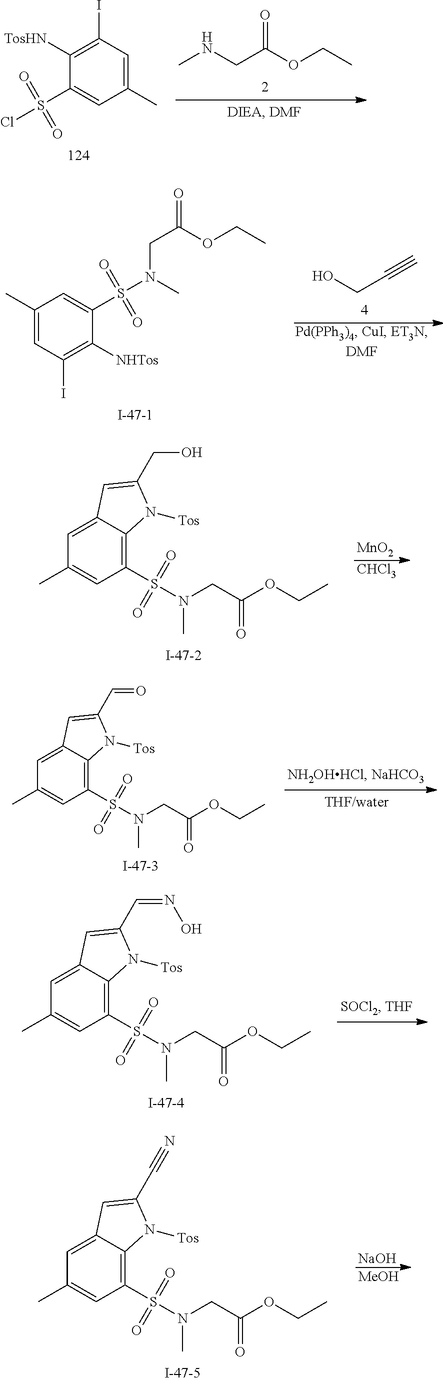

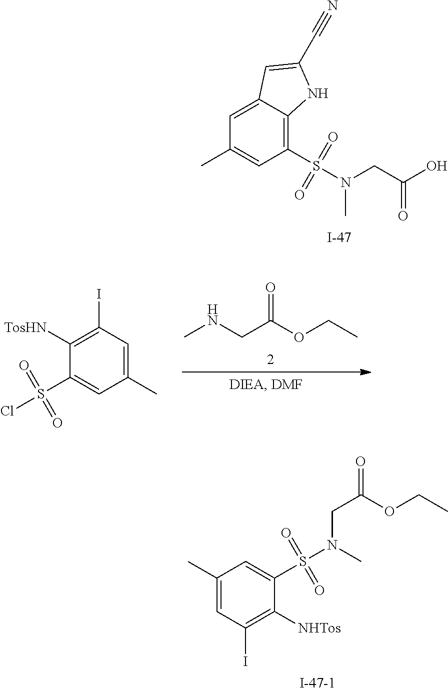

A mixture of ethyl methylglycinate as hydrogen chloride salt (0.505 g, 3.29 mmol) and diisopropylethylamine (1.28 g, 9.88 mmol) in dimethylformamide (10 mL) under nitrogen was stirred at 25° C. for 5 minutes. To this was added 124 (1.60 g, 3.29 mmol) and stirred for 55 minutes. The mixture was poured into ice-water (50 mL) and stirred for 5 minutes then extracted with ethyl acetate (100 mL*3). The combined organic layers were washed with brine (20 mL*3), dried over anhydrous sodium sulfate, filtered and the filtrate was concentrated under reduced pressure. The residue was purified by silica gel chromatography (SiO₂, petroleum ether/ethyl acetate=100/1 to 2/1) to afford ethyl N-((3-iodo-5-methyl-2-((4-methylphenyl)sulfonamido)phenyl)sulfonyl)-N-methylglycinate (I-47-1) (0.450 g, 23.6% yield) as a yellow solid.

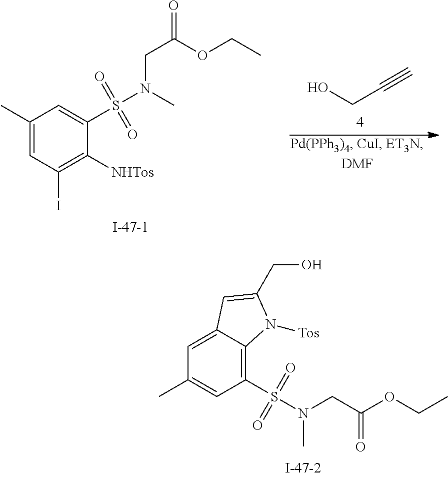

To a mixture of I-47-1 (0.200 g, 0.353 mmol), copper(I) iodide (0.007 g, 0.035 mmol), triethylamine (0.107 g, 1.06 mmol) and prop-2-yn-1-ol (4) (0.090 g, 1.61 mmol) in dimethylformamide (2 mL) was added Pd(PPh₃)₄ (0.040 g, 0.035 mmol) in one portion at 25° C. under nitrogen. The mixture was stirred at 90° C. for 20 hours. The mixture was poured into ice-water (20 mL), stirred for 5 minutes, then extracted with ethyl acetate (50 mL*3). The combined organic layers were washed with brine (20 mL*3), dried over anhydrous sodium sulfate, filtered, and the filtrate was concentrated under reduced pressure. The residue was purified by prep-TLC (SiO₂, petroleum ether/ethyl acetate=1/1) to afford ethyl N-((2-(hydroxymethyl)-5-methyl-1-tosyl-1H-indol-7-yl)sulfonyl)-N-methylglycinate (I-47-2) (0.100 g, 50.7% yield) as a yellow gum.

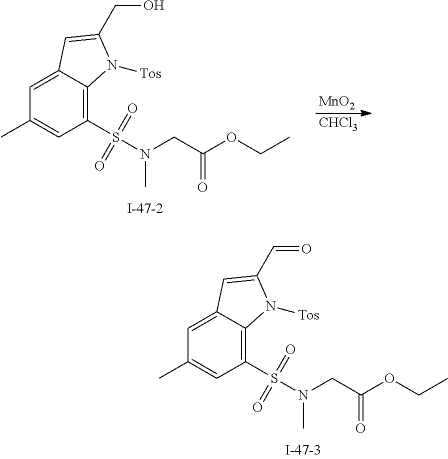

To a mixture of I-47-2 (0.050 g, 0.089 mmol) in chloroform (2 mL) was added manganese(IV) oxide (0.077 g, 0.896 mmol). The mixture was stirred at 70° C. for 20 hours then filtered and the filtrate was concentrated under reduced pressure to afford ethyl N-((2-formyl-5-methyl-1-tosyl-1H-indol-7-yl)sulfonyl)-N-methylglycinate (I-47-3) (0.040 g, 0.081 mmol, 90.5% yield) as a yellow solid. No further purification was performed.

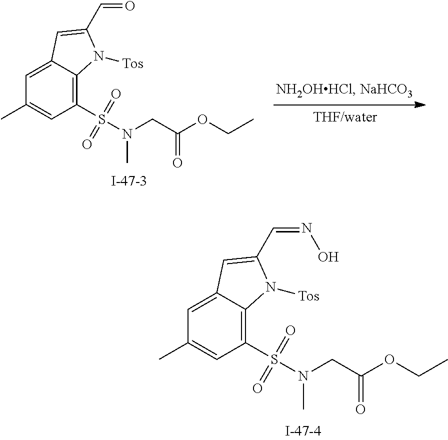

To a mixture of I-47-3 (0.040 g, 0.081 mmol) and hydroxylamine (0.028 g, 0.406 mmol) in tetrahydrofuran (1 mL)/water (0.1 mL) under nitrogen, was added sodium bicarbonate (0.034 g, 0.406 mmol). The mixture was stirred at 60° C. for 2 hours. The mixture was poured into ice-water (20 mL) and stirred for 5 minutes, then extracted with ethyl acetate (100 mL*3). The combined organic layers were washed with brine (20 mL*3), dried over anhydrous sodium sulfate, filtered, and the filtrate was concentrated under reduced pressure to affo106ydroxyamino-N-((2-((hydroxyimino)methyl)-5-methyl-1-tosyl-1H-indol-7-yl)sulfonyl)-N-methylglycinate (I-47-4) (0.080 g, 0.157 mmol) as a yellow solid. No further purification was performed.

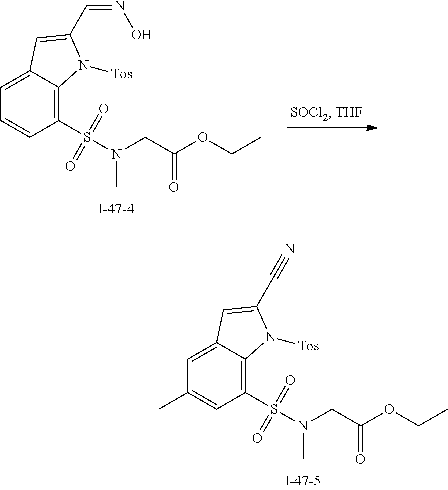

To a solution of ethyl I-47-4 (0.080 g, 0.157 mmol) in tetrahydrofuran (1 mL) was added thionyl chloride (0.037 g, 0.315 mmol) at 0° C. under nitrogen. The mixture was stirred at 25° C. for 1.5 hours then poured into ice-water (10 mL), stirred for 5 minutes, and extracted with ethyl acetate (50 mL*3). The combined organic layers were washed with brine (20 mL*2), dried over anhydrous sodium sulfate, filtered, and the filtrate was concentrated under reduced pressure. The residue was purified by prep-TLC (SiO₂, petroleum ether/ethyl acetate=1/1) to afford ethyl N-((2-cyano-5-methyl-1-tosyl-1H-indol-7-yl)sulfonyl)-N-methylglycinate (I-47-5) (0.060 g, 73.1% yield) as a yellow solid.

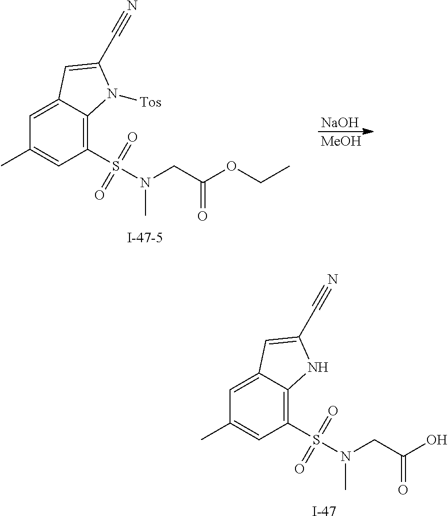

To a solution of I-47-5 (0.060 g, 0.12 mmol) in a mixture of methanol (1 mL)/water (0.5 mL) was added sodium hydroxide (0.019 g, 0.490 mmol). The mixture was stirred at 25° C. for 1 hour then concentrated under reduced pressure. The residue was purified by reversed-phase flash (0.1% NH₃H₂O/MeCN condition) to afford N-((2-cyano-5-methyl-1H-indol-7-yl)sulfonyl)-N-methylglycine (I-47) (0.020 g, 52% yield) as a white solid.

LCMS of I-47: RT=0.266 min, m/z=308.1 [M+H]⁺

¹H-NMR of I-47: (DMSO-d₆, 400 MHz) δ 7.78 (s, 1H), 7.65 (s, 1H), 7.41 (s, 1H), 7.17 (br. s, 1H), 3.92 (s, 2H), 2.65 (s, 3H), 2.49 (s, 3H)

### Synthesis of 4-(2-(dimethylamino)ethoxy)-3-methylaniline (Intermediate I-48)

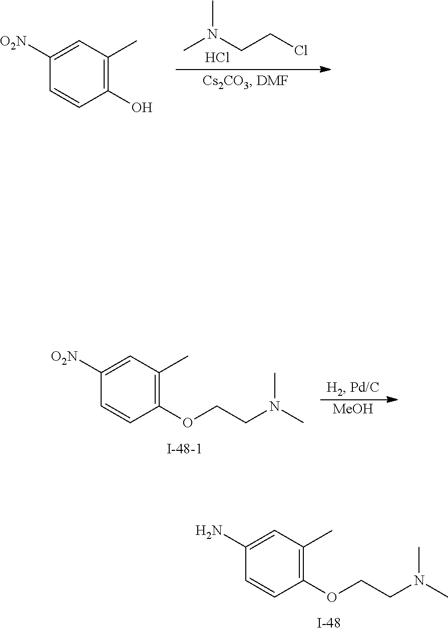

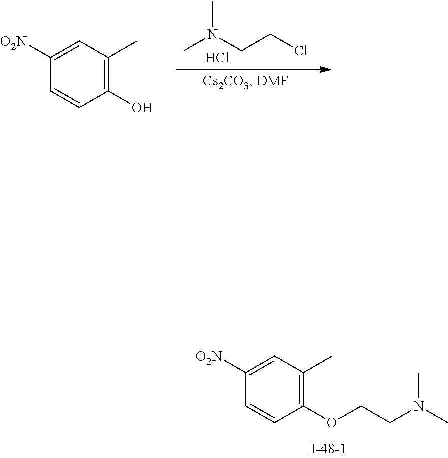

To a solution of 2-methyl-4-nitrophenol (3.00 g, 19.6 mmol) in dimethylformamide (30 mL) was added 2-chloro-N,N-dimethylethanamine hydrochloride (2.82 g, 19.6 mmol) and cesium carbonate (12.8 g, 39.2 mmol) at 25° C. The mixture was stirred at 70° C. for 2 hours then quenched by ice-water (100 mL) and extracted with ethyl acetate (200 mL*3). The combined organic layers were washed with brine (200 mL*2), dried over anhydrous sodium sulfate, filtered, and the filtrate was concentrated under reduced pressure. The residue was purified by column chromatography (SiO₂, petroleum ether/ethyl acetate=10/1 to 0/1, dichloromethane/methanol=10/1) to afford N,N-dimethyl-2-(2-methyl-4-nitrophenoxy)ethanamine (I-48-1) (1.00 g, 22.8% yield) as a brown oil.

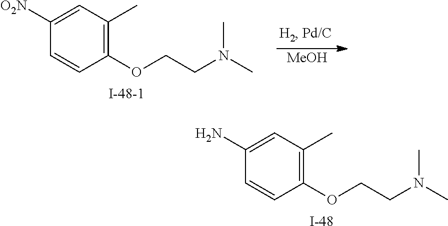

To a solution of I-48-1 (0.420 g, 1.87 mmol) in methanol (20 mL) was added Pd/C (0.050 g, 10% on charcoal, wet) at 25° C. under nitrogen atmosphere. The mixture was degassed and purged with hydrogen three times. The mixture was stirred at 25° C. under hydrogen atmosphere (15 Psi) for 2 hours. The reaction mixture was filtered and the filtrate was concentrated under reduced pressure to afford 4-(2-(dimethylamino)ethoxy)-3-methylaniline (I-48) (0.360 g, 97.6% yield) as a brown oil.

LCMS: RT=0.784 min, m/z 195.2 [M+H]⁺

¹H NMR: (DMSO-d₆, 400 MHz) δ 6.62 (d, J=8.4 Hz, 1H), 6.38 (d, J=2.4 Hz, 1H), 6.33 (dd, J1=2.8 Hz, J₂=8.4 Hz, 1H), 4.50 (br, s, 2H), 3.87 (t, J=6.0 Hz, 2H), 2.57 (t, J=6.0 Hz, 2H), 2.21 (s, 6H), 2.03 (s, 3H)

### Synthesis of (9H-fluoren-9-yl)methyl 4-(4-amino-2-chlorophenyl)piperidine-1-carboxylate (I-50)

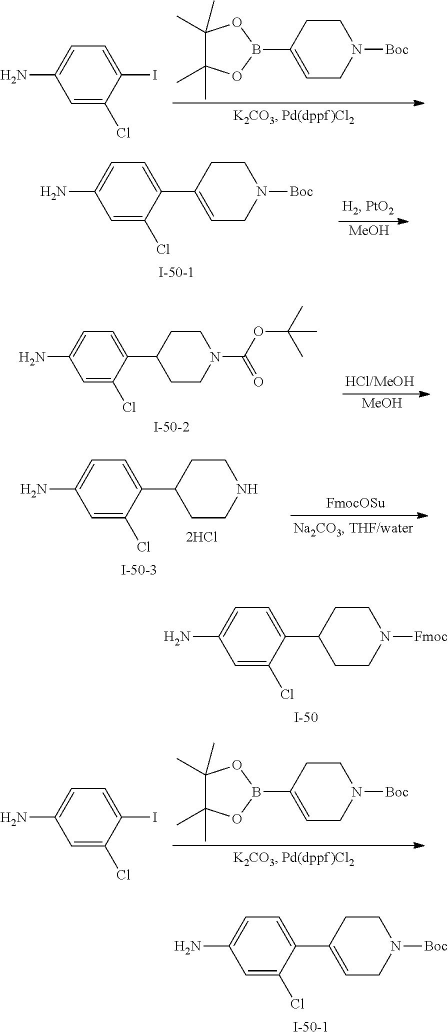

A mixture of 3-chloro-4-iodoaniline (2.00 g, 7.89 mmol), tert-butyl 4-(4,4,5,5-tetramethyl-1,3,2-dioxaborolan-2-yl)-5,6-dihydropyridine-1(2H)-carboxylate (2.44 g, 7.89 mmol), potassium carbonate (3.27 g, 23.7 mmol) and Pd(dppf)Cl₂ (0.289 g, 0.395 mmol) in dioxane (30 mL) and water (8 mL) was de-gassed then heated to 80° C. for 12 hours under nitrogen. The mixture was diluted with water (150 mL) and extracted with ethyl acetate (80 mL*2). The combined organic layers were washed with brine (150 mL), dried over anhydrous sodium sulfate, filtered, and the filtrate was concentrated under reduced pressure. The residue was purified by column chromatography (SiO₂, petroleum ether/ethyl acetate=30/1 to 5/1) to afford tert-butyl 4-(4-amino-2-chlorophenyl)-5,6-dihydropyridine-1(2H)-carboxylate (I-50-1) (2.40 g, 95.2% yield) as yellow oil.

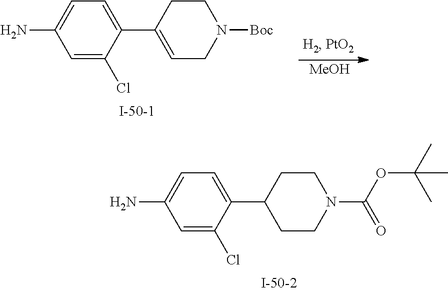 [SMILES: "MeOH" ; CC(C)(C)OC(=O)N1CC=C(c2ccc(N)cc2Cl)CC1 ; CC(C)(C)OC(=O)N1CCC(c2ccc(N)cc2Cl)CC1]

To a solution of I-50-1 (1.00 g, 3.24 mmol) in methanol (10 mL) was added PtO₂ (0.074 g, 0.324 mmol) under a nitrogen atmosphere. The suspension was degassed and purged with hydrogen several times. The mixture was stirred under hydrogen (15 psi) at 25° C. for 12 hours. The mixture was filtered and the filtrate was concentrated under reduced pressure to afford tert-butyl 4-(4-amino-2-chlorophenyl)piperidine-1-carboxylate (I-50-2) (0.900 g, 89.4% yield) as a white solid. No further purification was performed.

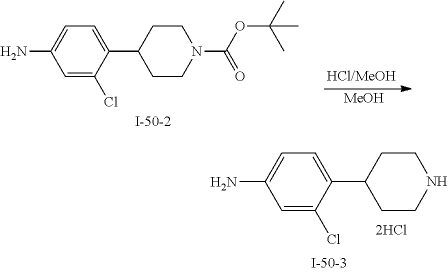 [SMILES: "MeOH" ; CC(C)(C)OC(=O)N1CCC(c2ccc(N)cc2Cl)CC1 ; Nc1ccc(C2CCNCC2)c(Cl)c1]

To a solution of I-50-2 (0.900 g, 2.90 mmol) in methanol (3 mL) was added a solution of HCl in methanol (4 M, 3 mL, 12 mmol). The mixture was stirred at 25° C. for 2 hours then concentrated under reduced pressure to afford 3-chloro-4-(piperidin-4-yl)aniline (I-50-3) as the di-hydrochloric salt (0.800 g, 97.4% yield) as a white solid. No further purification was performed.

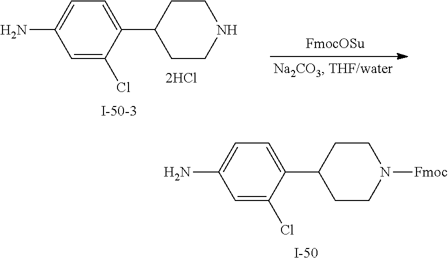

To a mixture of I-50-3 as the di-hydrochloric salt (0.800 g, 2.82 mmol) and sodium carbonate (0.474 g, 4.47 mmol) in tetrahydrofuran (8 mL) and water (8 mL) was added FMOC-OSU (1.14 g, 3.38 mmol). The mixture was stirred at 20° C. for 2.5 hours then diluted with water (50 mL) and extracted with ethyl acetate (20 mL*3). The combined organic layers were washed with brine (80 mL), dried over anhydrous sodium sulfate, filtered, and the filtrate was concentrated under reduced pressure. The residue was purified by column chromatography (SiO₂, petroleum ether/ethyl acetate=20/1 to 5/1) to afford (9H-fluoren-9-yl)methyl 4-(4-amino-2-chlorophenyl)piperidine-1-carboxylate (I-50) (0.900 g, 51.4% yield) as a colorless oil.

LCMS of I-50: RT=0.910 min, m/z 433.3 [M+H]⁺

¹H-NMR of I-50: (CD₃OD, 400 MHz) δ 7.80 (d, J-=7.2 Hz, 3H), 7.68-7.55 (m, 2H), 7.42-7.31 (m, 5H), 6.91 (d, J=8.4 Hz, 1H), 6.72 (d, J=2.0 Hz, 1H), 6.63 (dd, J₁=2.0 Hz, J₂-=8.4 Hz, 1H), 4.71-4.38 (m, 3H), 4.26-4.19 (m, 2H), 2.83-2.78 (m, 2H), 1.68-1.38 (m, 5H)

### Synthesis of 3-chloro-4-(2-(dimethylamino)ethoxy)aniline (I-51)

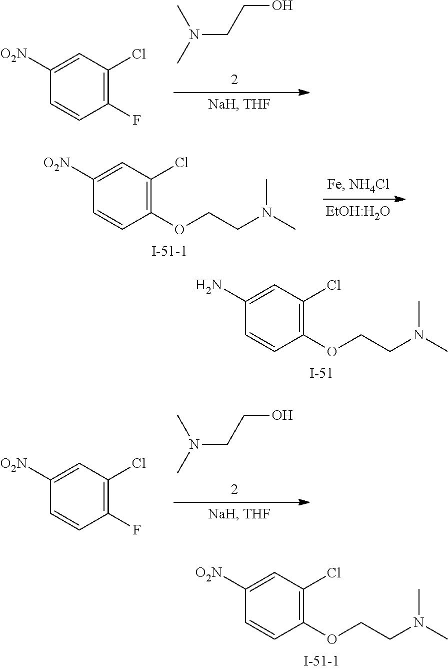

A solution of sodium hydride (3.42 g, 85.5 mmol, 60% in mineral oil) in tetrahydrofuran (150 mL) was stirred at 0° C. for 5 minutes. Then 2-(dimethylamino)ethan-1-ol (7.62 g, 85.5 mmol) was added dropwise at 0° C. and stirred at 10 minutes. To this was added 2-chloro-1-fluoro-4-nitrobenzene (10.0 g, 57.0 mmol) at 0° C. and the mixture stirred at 20° C. for 16 hours. The mixture was quenched with a saturated aqueous solution of ammonium chloride (150 mL) at 0° C., diluted with water (50 mL), and extracted with ethyl acetate (150 mL*2). The combined organic layers were washed with brine (40 mL), dried over anhydrous sodium sulfate, filtered, and the filtrate was concentrated under reduced pressure to afford 2-(2-chloro-4-nitrophenoxy)-N,N-dimethylethan-1-amine (I-51-1) (15.0 g, 78.6% yield) as red oil. No further purification was performed.

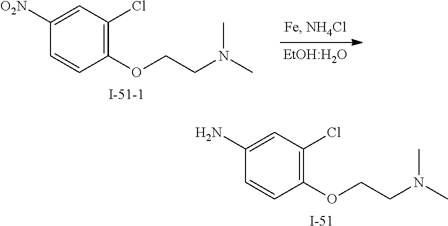

To a solution of I-51-1 (15.0 g, 44.8 mmol) in a mixture of ethanol (100 mL) and water (100 mL) was added iron (12.5 g, 224 mmol) and ammonium chloride (12.0 g, 224 mmol). The mixture was stirred at 80° C. for 2 hours under a N₂ atmosphere then filtered and the filtrate was concentrated under reduced pressure. The residue was diluted with water (100 mL) and extracted with ethyl acetate (150 mL*3). The combined organic layers were washed with brine (200 mL), dried over anhydrous sodium sulfate, filtered, and the filtrate was concentrated under reduced pressure. The crude product was purified by reversed-phase flash (0.1% FA/MeCN condition) to afford 3-chloro-4-(2-(dimethylamino)ethoxy)aniline (I-51) (7.60 g, 66.5% yield) as red oil.

LCMS: RT=0.847 min, m/z 215.2 [M+H]⁺

¹H NMR: (DMSO-d₆, 400 MHz) δ 6.85 (d, J=8.8 Hz, 1H), 6.62 (d, J=2.4 Hz, 1H), 6.47 (dd, J₁=2.4 Hz, J₂=8.4 Hz, 1H), 4.89 (s, 2H), 3.94 (t, J=5.6 Hz, 2H), 2.57 (t, J=5.6 Hz, 2H), 2.21 (s, 6H)

### Synthesis of 2-cyano-5-methyl-1H-indole-7-sulfonyl chloride (I-53)

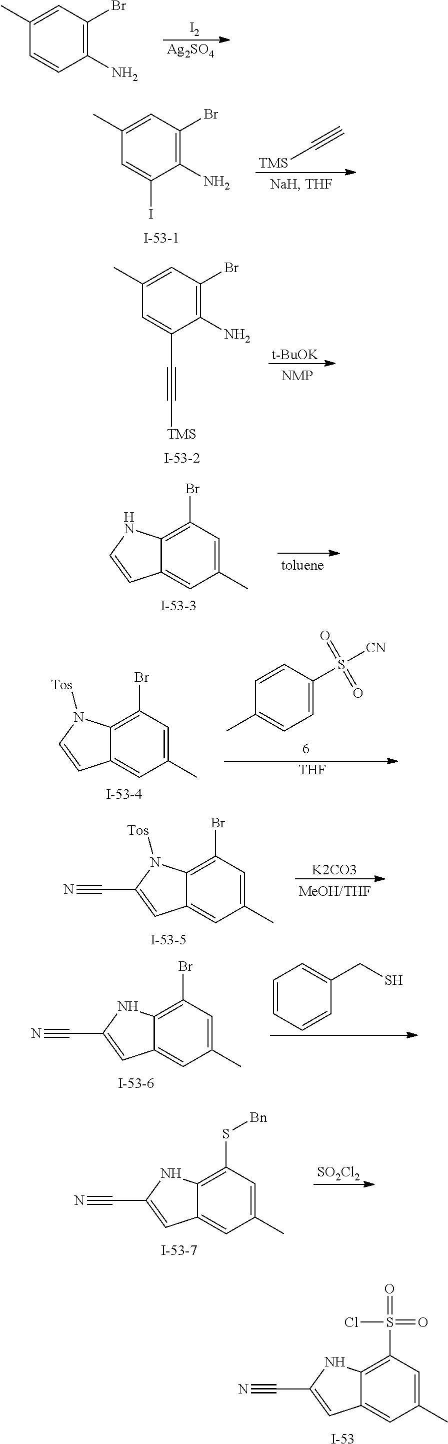

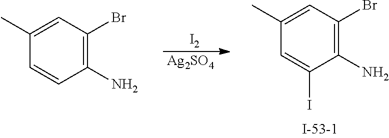

To a solution of 2-bromo-4-methylaniline (66.0 g, 355 mmol) in ethanol (660 mL) was added Ag₂SO₄ (66.1 mL, 390 mmol) followed by 12 (99.0 g, 390 mmol) in portions at 20° C. The mixture was stirred at 20° C. for 2 hours then diluted with a saturated aqueous solution of sodium sulfite (1 L), filtered, and the filtrate was extracted with ethyl acetate (500 mL*3). The combined organic layers were dried over anhydrous sodium sulfate, filtered, and the filtrate was concentrated under reduced pressure. The residue was purified by silica gel column (petroleum ether:ethyl acetate=1:0 to 10:1) followed by triturated with petroleum ether (400 mL), filtered and the cake was dried under reduced pressure to afford 2-bromo-6-iodo-4-methylaniline (I-53-1) (36.0 g, 32.3% yield) as a yellow solid. The filtrate was concentrated under reduced pressure to afford an additional amount of 2-bromo-6-iodo-4-methylaniline (I-53-1) (40.0 g, 128 mmol, 36.2% yield) as a red solid.

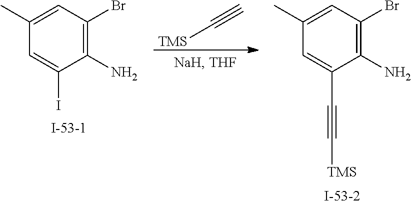

To a solution of I-53-1 (21.0 g, 67.3 mmol) in tetrahydrofuran (210 mL) was added ethynyltrimethylsilane (7.93 g, 80.8 mmol) followed by triethylamine (28.1 mL, 201 mmol), Pd(PPh₃)₂Cl₂ (1.89 g, 2.69 mmol) and CuI (1.03 g, 5.39 mmol). The mixture was stirred at 20° C. for 2 hours under nitrogen atmosphere. Water (500 mL) was added into the above reaction mixture and extracted with ethyl acetate (200 mL*2). The combined organic layers were dried over anhydrous sodium sulfate, filtered, and the filtrate was concentrated under reduced pressure. The residue was purified by silica gel column chromatography (petroleum ether:ethyl acetate=1:0 to 1:0) to afford 2-bromo-4-methyl-6-((trimethylsilyl)ethynyl)aniline (I-53-2) (21.0 g) as a yellow oil.

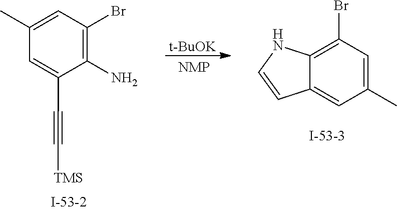

To a solution of I-53-3 (34.0 g, 120 mmol) in azomethylpyrrolidone (340 mL) was added potassium tert-butoxide (40.5 g, 361 mmol). The mixture was then stirred at 80° C. for 12 hours. Water (500 mL) was added into the above reaction mixture and extracted with ethyl acetate (200 mL*2). The combined organic layers were dried over anhydrous sodium sulfate, filtered, and the filtrate was concentrated under reduced pressure. The residue was purified by silica gel column (petroleum ether:ethyl acetate=1:0 to 10:1) to afford 7-bromo-5-methyl-1H-indole (I-53-3) (21.0 g, 76.8% yield) as a yellow oil.

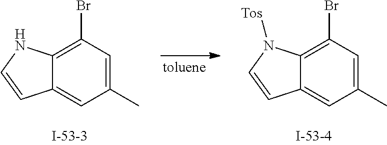

To a solution of I-53-3 (21.0 g, 99.9 mmol) in toluene (600 mL) was added TosCl (28.6 g, 150 mmol), hydrogen sulfate; tetrabutylammonium (3.39 g, 10.0 mmol), then sodium hydroxide (80.0 g, 1.00 mol, 50% purity in water) was dropped at 0° C. The mixture was stirred at 20° C. for 3 hours. The mixture was poured into ice-water (1 L), extracted with ethyl acetate (500 mL*3). The combined organic layers were washed with brine (500 mL*2), dried over anhydrous sodium sulfate, filtered and the filtrate was concentrated under reduced pressure. The residue was purified by silica gel column (petroleum ether:ethyl acetate=1:0 to 1:1), followed by triturated with methyl tert-butyl ether (40 mL), filtered to afford 7-bromo-5-methyl-1-tosyl-1H-indole (I-53-4) (15.0 g, 31.9% yield) as yellow solid. The filtrate was concentrated under reduced pressure to afford an additional batch of 7-bromo-5-methyl-1-tosyl-1H-indole (I-53-4) (18.0 g, 49.4% yield) as a red oil.

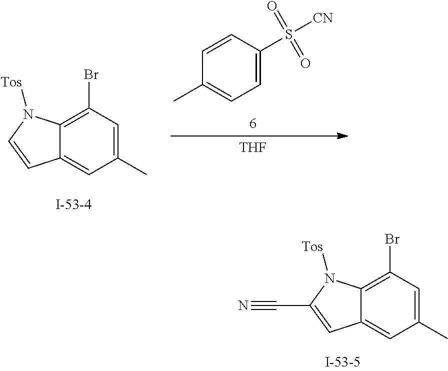

To a solution of I-53-4 (4.40 g, 12.1 mmol) in tetrahydrofuran (90 mL) was added LDA (2 M, 8.46 mL) at −70° C. over 10 minutes. Then a solution of p-tolylsulfonylformonitrile (2.19 g, 12.1 mmol) in tetrahydrofuran (15 mL) was added to the mixture at −70° C. The result mixture was stirred at −40° C. for 40 minutes. The mixture was quenched with a saturated aqueous solution of ammonium chloride (300 mL) and extracted with ethyl acetate (150 mL*3). The combined organic layers were washed with brine (300 mL), dried over anhydrous sodium sulfate, filtered, and the filtrate was concentrated under reduced pressure. The residue was purified by silica gel chromatography (petroleum ether/ethyl acetate=1:0˜10:1) to afford 7-bromo-5-methyl-1-tosyl-1H-indole-2-carbonitrile (I-53-5) (9.00 g, 54.7% yield) as a brown solid

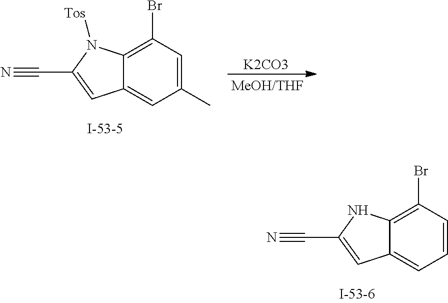

To a solution of I-53-5 (4.00 g, 5.87 mmol) in a mixture of methanol (30 mL) and tetrahydrofuran (30 mL) was added potassium carbonate (1.62 g, 11.8 mmol) at 25° C. The mixture was stirred at 25° C. for 1 hour then quenched with water (100 mL) and extracted with ethyl acetate (80 mL*3). The combined organic layers were washed with brine (100 mL), dried over anhydrous sodium sulfate, filtered, and the filtrate was concentrated under reduced pressure. The residue was purified by silica gel chromatography (petroleum ether/ethyl acetate=1:0˜10:1) to afford 7-bromo-5-methyl-1H-indole-2-carbonitrile (I-53-6) (1.70 g, 85.7% yield) as a white solid.

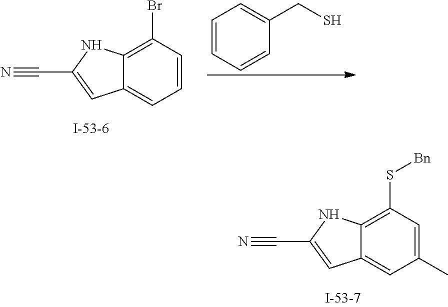

To a solution of I-53-6 (1.70 g, 5.03 mmol) in dioxane (20 mL) was added Xantphos (0.582 g, 1.01 mmol), diisopropylethylamine (3.07 mL, 17.6 mmol), Pd₂(dba)₃ (0.461 g, 0.503 mmol) and phenylmethanethiol (0.730 g, 5.88 mmol) at 25° C. The mixture was stirred at 85° C. for 12 hours then diluted with water (100 mL) and extracted with ethyl acetate (80 mL*3). The combined organic layers were washed with brine (150 mL), dried over sodium sulfate, filtered, and the filtrate was concentrated under reduced pressure. The residue was purified by silica gel chromatography (petroleum ether/ethyl acetate=1:0˜20:1) to afford 7-(benzylthio)-5-methyl-1H-indole-2-carbonitrile (I-53-7) (1.70 g, 91.0% yield) as yellow oil.

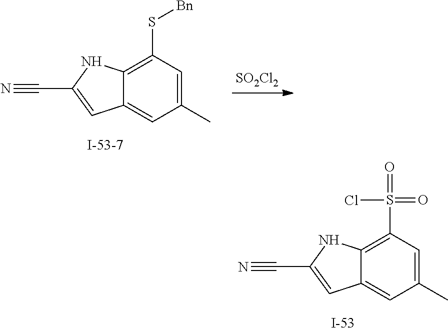

To a solution of I-53-7 (0.500 g, 1.35 mmol) in a mixture of acetonitrile (10 mL), water (1 mL) and acetic acid (2 mL) was added sulfuryl chloride (0.161 mL, 1.62 mmol) at 0° C. The mixture was stirred at 0° C. for 30 minutes then diluted with water (30 mL) and extracted with ethyl acetate (30 mL*3). The combined organic layers were washed with brine (20 mL), dried over anhydrous sodium sulfate, filtered, and the filtrate was concentrated under reduced pressure to afford 2-cyano-5-methyl-1H-indole-7-sulfonyl chloride (I-53) (0.450 g, crude) as a yellow solid. No further purification was performed.

### Synthesis of N-((2-cyano-5-methyl-1-tosyl-1H-indol-7-yl)sulfonyl)-N-methylglycine (I-58)

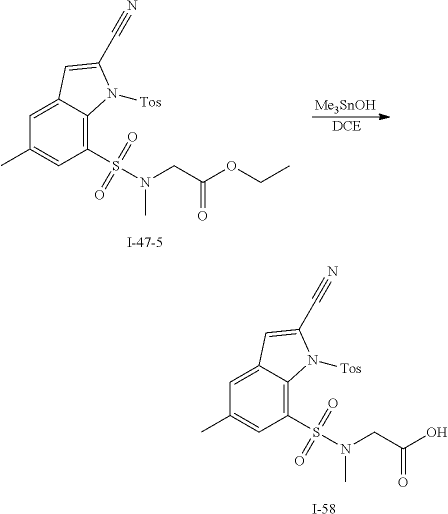

To a mixture of I-47-5 (0.180 g, 0.367 mmol) in dichloroethane (5 mL) was added hydroxy(trimethyl)stannane (0.199 g, 1.10 mmol) 25° C. The mixture was stirred at 80° C. for 2 hours. The mixture was poured into ice-water (5 mL) and stirred for 5 minutes. The aqueous phase was adjusted to approximately pH-6 by 1N hydrochloric acid solution then extracted with dichloromethane (20 mL*3). The combined organic layers were washed with brine (10 mL*2), dried over anhydrous sodium sulfate, filtered, and the filtrate was concentrated under reduced pressure to afford N-((2-cyano-5-methyl-1-tosyl-1H-indol-7-yl)sulfonyl)-N-methylglycine (I-58) (0.200 g) as a yellow solid. No further purification was performed.

LCMS of I-58: RT=0.619 min, m/z=462.1 [M+H]⁺

¹H-NMR of I-58: (CDCl₃, 400 MHz) δ 8.08 (s, 1H), 7.80 (d, J=8.4 Hz, 2H)-7.48 (s, 1H), 7.27-7.22 (m, 3H), 4.10 (s, 2H), 2.82 (s, 3H), 2.51 (s, 3H), 2.40 (s, 3H)

### Synthesis of N-(2-methoxypyridin-4-yl)-2-(methylamino)acetamide (Intermediate I-59)

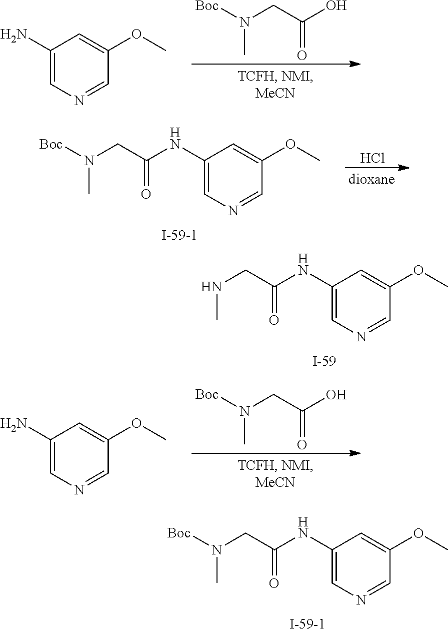

To a solution of 2-((tert-butoxycarbonyl)(methyl)amino)acetic acid (0.460 g, 2.42 mmol) in acetonitrile (10 mL) was added 1-methylimidazole (0.890 g, 10.9 mmol). To the mixture was added TCFH (1.02 g, 3.62 mmol) followed by 2-methoxypyridin-4-amine (0.300 g, 2.42 mmol) at 0° C. The reaction solution was stirred at 40° C. for 1 hour under nitrogen then purified by reversed-phase flash (0.1% FA/MeCN condition) to afford tert-butyl (2-((2-methoxypyridin-4-yl)amino)-2-oxoethyl)(methyl)carbamate (I-59-1) (1.70 g, 46.1% yield) as brown oil.

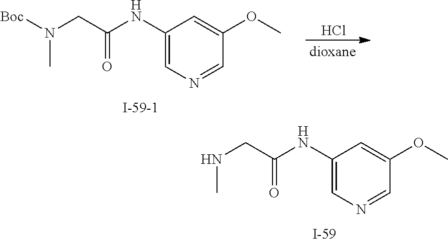

To a solution of I-59-1 (0.237 g, 0.800 mmol) in methanol (2 mL) was added a solution of hydrochloric acid in dioxane (4 mol/L, 2 mL, 8 mmol). The reaction solution was stirred at 25° C. for 2 hours then concentrated under reduced pressure to afford N-(2-methoxypyridin-4-yl)-2-(methylamino)acetamide (I-59) as a hydrochloric salt (0.180 g, 96.8% yield) as a white solid.

¹H NMR of I-59: (CDCl₃, 400 MHz) δ 11.-2 (br. s, 1H), 9.25-9.24 (s, 2H), 8.13 (d, J-=6.0 Hz, 1H), 7.32-7.30 (m, 2H), 4.03-4.01 (t, J-=5.2 Hz, 3H), 3.94-3.90 (s, 3H), 2.62-2.59 (t, J=5.2 Hz, 3H)

### Synthesis of N-(3-chloro-4-cyclopropylphenyl)-2-(methylamino)acetamide (Intermediate I-60)

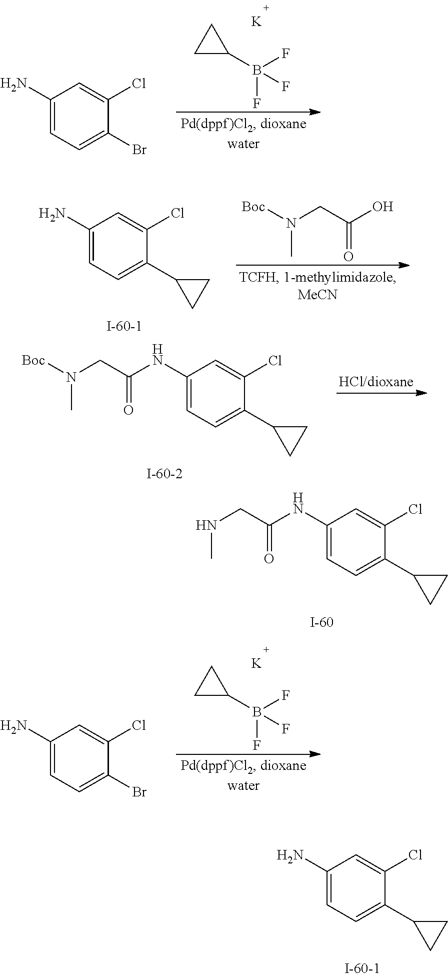

To a solution of 4-bromo-3-chloro-aniline (1.00 g, 4.84 mmol) and potassium; cyclopropyl(trifluoro)boranuide (1.43 g, 9.69 mmol) in water (3 mL) and dioxane (10 mL) was added sodium carbonate (1.54 g, 14.5 mmol) at 25° C. under nitrogen. Then Pd(dppf)Cl₂ (0.354 g, 0.480 mmol) was added to the mixture. The mixture was degassed and purged with nitrogen for three times. The resulted mixture was stirred at 100° C. for 12 hours under nitrogen atmosphere. The reaction mixture was diluted with water (10 mL) and extracted with ethyl acetate (15 mL*4). The combined organic phases were washed with brine (10 mL*2), dried over anhydrous sodium sulfate, filtered, and the filtrate was concentrated under reduced pressure. The residue was purified by column chromatography (silica gel, Petroleum ether/Ethyl acetate=1:0 to 40:1) to afford 3-chloro-4-cyclopropylaniline (I-60-1) (0.475 g, 58.5% yield) as a yellow oil.

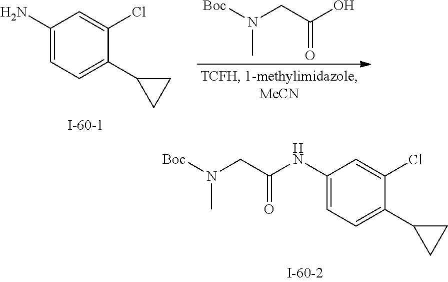 [SMILES: Nc1ccc(C2CC2)c(Cl)c1 ; CN(CC(=O)O)C(=O)OC(C)(C)C ; CN(CC(=O)Nc1ccc(C2CC2)c(Cl)c1)C(=O)OC(C)(C)C]

To a solution of 2-[tert-butoxycarbonyl(methyl)amino]acetic acid (0.205 g, 1.08 mmol) and 1-methylimidazole (0.445 g, 5.42 mmol) in acetonitrile (20 mL) was added TCFH (0.608 g, 2.17 mmol) at 0° C. To the mixture was added I-60-1 (0.200 g, 1.19 mmol) at 25° C. The mixture was stirred at 40° C. for 0.5 hour. The reaction mixture was diluted with water (15 mL) and extracted with ethyl acetate (15 mL*4). The combined organic phases were washed with brine (10 mL*2), dried over anhydrous sodium sulfate, filtered, and the filtrate was concentrated under reduced pressure. The residue was purified by reversed-phase flash (0.1% NH₃H₂O/MeCN condition) to afford tert-butyl N-[2-(3-chloro-4-cyclopropyl-anilino)-2-oxo-ethyl]-N-methyl-carbamate (I-60-2) (0.240 g, 65.3% yield) as a yellow gum.

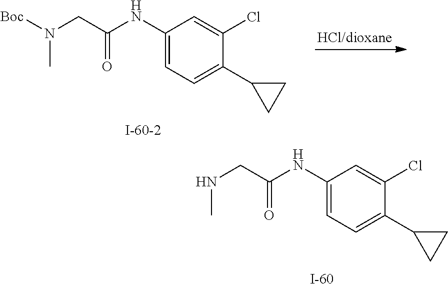

To a solution of I-60-2 (0.240 g, 0.708 mmol) in dioxane (2 mL) was added a solution of hydrochloric acid in dioxane (4 mol/L, 2.4 mL). The mixture was stirred at 25° C. for 30 minutes then concentrated under reduced pressure to afford N-(3-chloro-4-cyclopropylphenyl)-2-(methylamino)acetamide (I-60) hydrochloric salt (0.130 g, 66.7% yield) as a white solid.

¹H-NMR of I-60: (DMSO-d₆ 400 MHz) δ 10.69 (br. s, 1H), 8.85 (br. s, 2H), 7.76 (d, J-=2.0 Hz, 1H), 7.38-7.36 (dd, J₁₌2.0 Hz, J₂=2.4 Hz, 1H), 7.02 (d, J=8.4 Hz, 1H)-3.92 (s, 2H), 2.54-2.51 (m, 3H), 2.08-2.03 (m, 1H), 1.02-0.93 (m, 2H), 0.68-0.64 (m, 2H)

### Synthesis of 2-cyano-5-methyl-1-tosyl-1H-indole-7-sulfonyl chloride (Intermediate I-66)

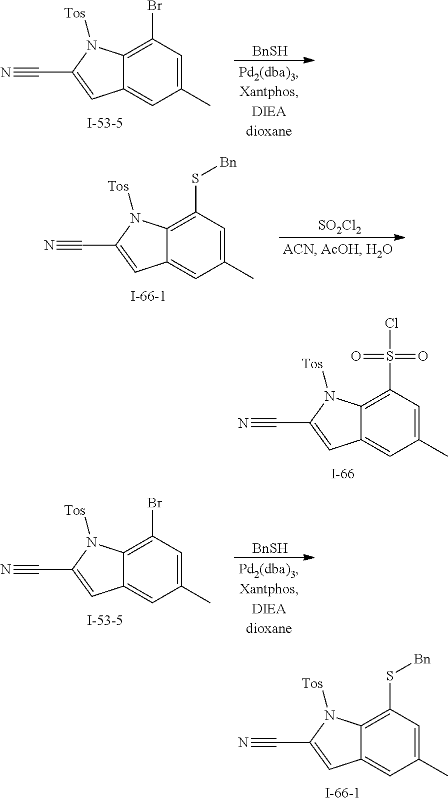

To a solution of I-53-5 (27.0 g, 69.4 mmol) in dioxane (300 mL) was added N-ethyl-N-isopropylpropan-2-amine (44.8 g, 347 mmol), Pd₂(dba)₃ (6.35 g, 6.94 mmol), Xantphos (8.03 g, 13.9 mmol) and phenylmethanethiol (12.5 g, 101 mmol) at 25° C. The mixture was stirred at 95° C. for 12 hours under nitrogen then diluted with water (800 mL) and extracted with ethyl acetate (600 mL*3). The combined organic layers were washed with brine (500 mL*3), dried over anhydrous sodium sulfate, filtered, and filtrate was concentrated under reduced pressure. The residue was purified by column chromatography (silica gel, petroleum ether/ethyl acetate=15/1 to 8/1) and then re-purified by reversed-phase flash (0.1% FA/acetonitrile condition) to afford 7-(benzylthio)-5-methyl-1-tosyl-1H-indole-2-carbonitrile (I-66-1) (21.0 g, 70.0% yield) as a yellow solid.

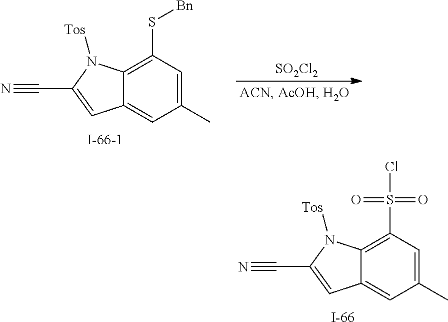

To a solution of I-66-1 (5.00 g, 11.6 mmol) in acetonitrile (32 mL), water (3.2 mL) and acetic acid (6.4 mL) was added sulfuryl chloride (5.46 g, 40.5 mmol) at 0° C., and the mixture was stirred at 0° C. for 1 hour. The mixture was added into water (50 mL), causing a solid to precipitate. The mixture was filtered and filter cake was collected to afford 2-cyano-5-methyl-1-tosyl-1H-indole-7-sulfonyl chloride (I-66) (4.70 g, 99.4% yield) as a yellow solid which was used into next step directly without further purification.

### Synthesis of 5-chloro-2-cyano-1-tosyl-1H-indole-7-sulfonyl chloride (Intermediate I-68)

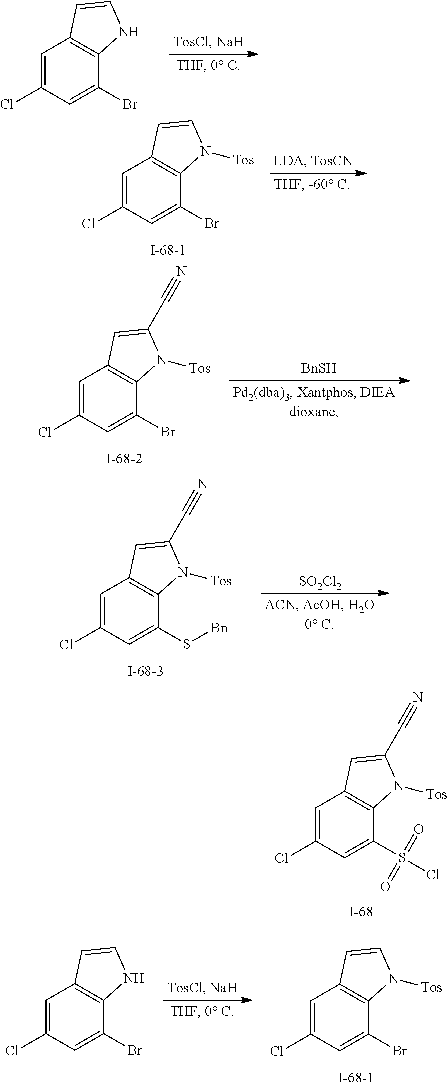

To a solution of 7-bromo-5-chloro-1H-indole (3.90 g, 16.9 mmol) in tetrahydrofuran (15 mL) was added sodium hydride (0.880 g, 22.0 mmol, 60% in mineral oil) in portions at 0° C. then the solution was stirred at 0° C. for 1 hour. After that, 4-methylbenzene-1-sulfonyl chloride (3.71 g, 19.5 mmol) was added to the mixture at 0° C. and stirred for 1 hour. The reaction mixture was quenched by addition saturated aqueous solution of ammonium chloride (30 mL) at 0° C., extracted with ethyl acetate (30 mL*3). The combined organic layers were washed with brine (50 mL*3), dried over sodium sulfate, filtered, and the filtrate was concentrated under reduced pressure to give a residue. The residue was purified by column chromatography (silica gel, petroleum ether/ethyl acetate=50/1 to 5/1) to afford 7-bromo-5-chloro-1-tosyl-1H-indole (I-68-1) (2.83 g, 7.36 mmol, 43.5% yield) as a blue solid.

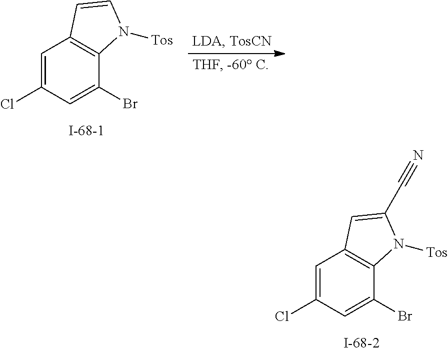

To a solution of I-68-1 (2.83 g, 7.36 mmol) in tetrahydrofuran (40 mL) was added lithium diisopropylamide (4.42 mL, 2 mol/L in tetrahydrofuran) at −60° C. After 30 minutes, 4-methylbenzenesulfonyl cyanide (1.33 g, 7.36 mmol) was added to the reaction at −60° C. and stirred for 1 hour. The reaction mixture was quenched by addition saturated aqueous solution of ammonium chloride (50 mL) at 0° C., extracted with ethyl acetate (50 mL*3). The combined organic layers were washed with brine (100 mL*3), dried over sodium sulfate, filtered, and the filtrate was concentrated under reduced pressure to give a residue. The residue was purified by column chromatography (silica gel, petroleum ether/ethyl acetate=10/1 to 5/1) to afford 7-(benzylthio)-5-chloro-1-tosyl-1H-indole-2-carbonitrile (I-68-2) (1.07 g, 2.61 mmol, 35.5% yield) as a white solid.

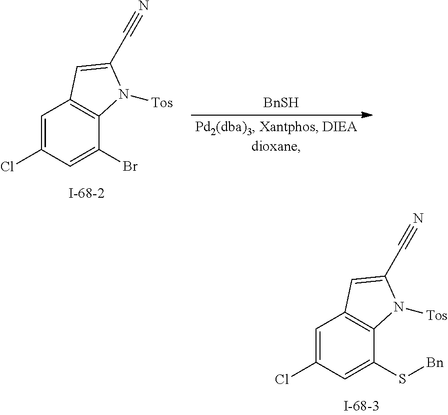

A mixture of I-68-2 (1.07 g, 2.61 mmol), phenylmethanethiol (0.389 g, 3.13 mmol), tris(dibenzylideneacetone)dipalliadium (0.239 g, 0.261 mmol), (9,9-dimethyl-9H-xanthene-4,5-diyl)bis(diphenylphosphine) (0.302 g, 0.522 mmol) and N-ethyl-N-isopropylpropan-2-amine (1.69 g, 13.1 mmol) in dioxane (15 mL) was degassed and purged with nitrogen for three times, and then the mixture was stirred at 95° C. for 12 hours under nitrogen atmosphere. The reaction mixture was quenched by addition water (20 mL), and then extracted with ethyl acetate (20 mL*3). The combined organic layers were washed with brine (40 mL*3), dried over sodium sulfate, filtered and the filtrate was concentrated under reduced pressure to give a residue. The residue was purified by column chromatography (silica gel, petroleum ether/ethyl acetate=40/1 to 5/1) to afford 7-(benzylthio)-5-chloro-1-tosyl-1H-indole-2-carbonitrile (I-68-3) (0.910 g, 2.01 mmol, 76.9% yield) as a yellow solid.

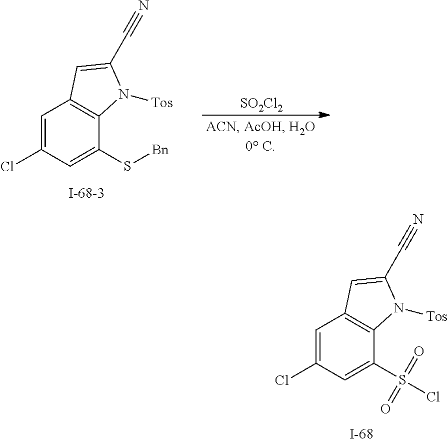

To a solution of I-68-3 (0.400 g, 0.883 mmol) in acetonitrile (10 mL), acetate acid (1 mL) and water (0.5 mL) was added sulfuryl chloride (0.596 g, 4.42 mmol). The mixture was stirred at 0° C. for 0.5 hour. After that the reaction mixture was poured into ice-water (10 mL) at 0° C., causing a solid to precipitate out. Then the suspension was filtered and the filter cake was collected to afford 5-chloro-2-cyano-1-tosyl-1H-indole-7-sulfonyl chloride (I-68) (0.240 g, 0.559 mmol, 63.3% yield) as a white solid.

¹H-NMR of I-68: (DMOS-d₆, 400 MHz) δ 7.85 (d, J=2.0 Hz, 1H), 7.79 (s, 1H), 7.73 (d, J=8.4 Hz, 2H), 7.61 (d, J=4.4 Hz, 2H), 6.89 (d, J=2.4 Hz, 1H), 2.34 (s, 3H)

### Synthesis of N-(but-2-yn-1-yl)-N-methyl-2-(methylamino)acetamide (Intermediate I-69)

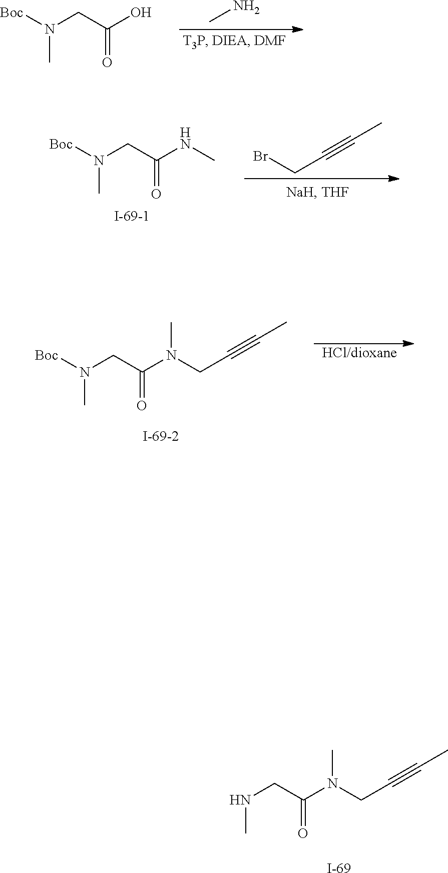

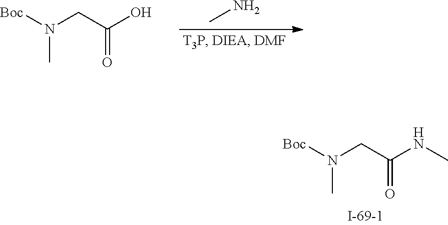

To a solution of 2-((tert-butoxycarbonyl)(methyl)amino)acetic acid (3.00 g, 15.9 mmol) in N,N-dimethylformamide (30 mL) was added diisopropylethylamine (10.3 g, 79.3 mmol), methylamine (1.07 g, 15.86 mmol, as a hydrochloric salt) and T₃P (20.2 g, 31.7 mmol, 50% w/w in ethyl acetate) at 0° C. The mixture was stirred at 25° C. for 3 hours then diluted with water (100 mL) and extracted with ethyl acetate (100 mL*2). The combined organic layers were washed with brine (100 mL*2), dried over anhydrous sodium sulfate, filtered, and the filtrate was concentrated under reduced pressure to give a residue. The residue was purified by column chromatography (silica gel, Petroleum ether/Ethyl acetate=99/1 to 65/35) to afford tert-butyl methyl(2-(methylamino)-2-oxoethyl)carbamate (I-69-1) (1.60 g, 7.91 mmol, 49.9% yield) as a white solid.

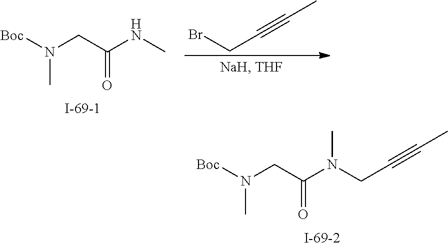

To a solution of I-69-1 (1.50 g, 7.42 mmol) in tetrahydrofuran (20 mL) was added sodium hydride (0.593 g, 14.8 mmol, 60% in mineral oil) at 0° C. After 30 minutes 1-bromobut-2-yne (0.986 g, 7.42 mmol) was added at 0° C. and the mixture was stirred at 0° C. for 12 hours. After that the reaction mixture was quenched with saturated aqueous solution of ammonia chloride (80 mL) and extracted with ethyl acetate (60 mL*2). The combined organic layers were washed with brine (60 mL*2), dried over anhydrous sodium sulfate, filtered, and the filtrate was concentrated under reduced pressure to give a residue. The residue was purified by column chromatography (silica gel, Petroleum ether/Ethyl acetate=99/1 to 60/40) to afford tert-butyl (2-(but-2-yn-1-yl(methyl)amino)-2-oxoethyl)(methyl)carbamate (I-69-2) (0.630 g, 2.48 mmol, 33.4% yield) as yellow oil.

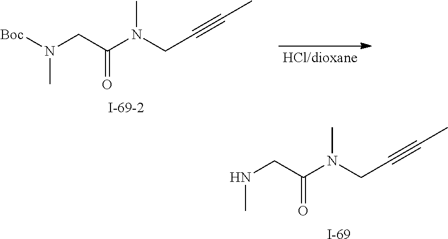

To a solution of I-69-2 (0.630 g, 2.48 mmol) in dioxane (5 mL) was added a solution of hydrochloric acid in dioxane (4 mol/L, 10 mL), then mixture was stirred at 25° C. for 0.5 hour. Then mixture was concentrated under reduced pressure to afford N-(but-2-yn-1-yl)-N-methyl-2-(methylamino)acetamide (I-69) as a hydrochloric salt (0.500 g, crude) as a yellow solid.

¹H-NMR of I-69: (DMSO-d₆, 400 MHz) δ 9.03 (br. s, 2H), 4.14-4.11 (m, 2H), 4.02 (s, 2H), 2.95-2.90 (m, 3H), 2.57-2.52 (m, 3H), 1.84-1.78 (m, 3H)

### Synthesis of 2-(methylamino)-N-(2-methylpent-3-yn-2-yl)acetamide (Intermediate I-72)

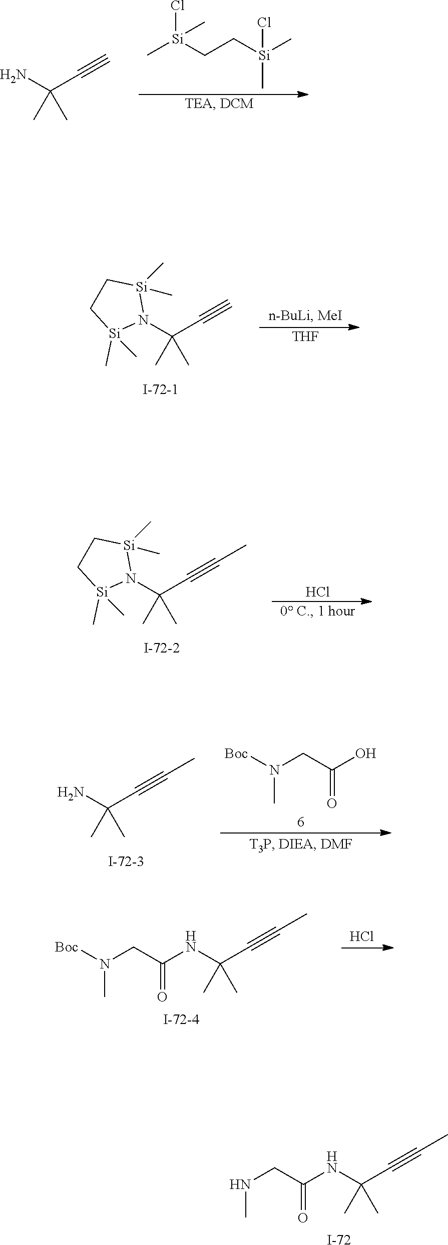

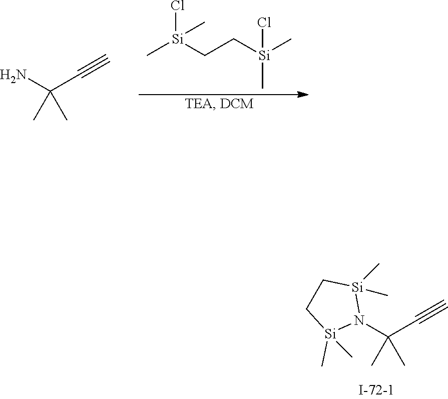

To a solution of 2-methylbut-3-yn-2-amine (10.00 g, 120.3 mmol) in dichloromethane (100 mL) was added triethylamine (36.52 g, 360.9 mmol) and chloro-[2-[chloro(dimethyl)silyl]ethyl]-dimethyl-silane (25.89 g, 120.3 mmol) at 0° C. The mixture was stirred at 25° C. for 12 hours under N₂ then filtered and the filtrate was concentrated under reduced pressure. The crude product was distilled at 120° C. under reduced pressure (pressure: 0.02 Mpa) to afford 2,2,5,5-tetramethyl-1-(2-methylbut-3-yn-2-yl)-1,2,5-azadisilolidine (I-72-01) (8.00 g, 35.5 mmol, 29.5% yield) as colorless oil.

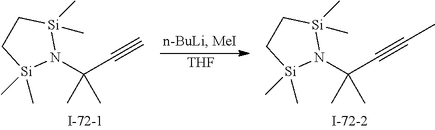

To a solution of I-72-1 (1.00 g, 4.44 mmol) in tetrahydrofuran (10 mL) was added n-butyllithium (2.5 mol/L in hexane, 2.13 mL) dropwise at −70° C. After 1 hour, iodomethane (0.630 g, 4.44 mmol) was added into the mixture at 0° C., and the mixture was stirred at 20° C. for 1 hour. Then the mixture was added into water (50 mL, 5° C.) and extracted with ethyl acetate (30 mL*3). The combined organic phase was washed with brine (30 mL*3), dried over anhydrous sodium sulfate, filtered, and filtrate was concentrated under reduced pressure to afford 2,2,5,5-tetramethyl-1-(2-methylpent-3-yn-2-yl)-1,2,5-azadisilolidine (I-72-2) (1.00 g, crude) as colorless oil. No further purification was performed.

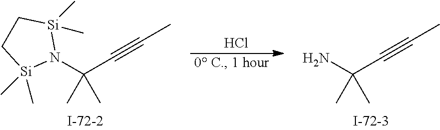

To a solution of I-72-2 (1.00 g, 4.18 mmol) in dioxane (10.00 mL) was added a 4 M solution of hydrochloric acid in dioxane and the mixture was stirred at 0° C. for 1 hour. Then the mixture was added into water (20 mL) and extracted with ethyl acetate (20 mL*2). The aqueous phase was lyophilized to afford 2-methylpent-3-yn-2-amine as a hydrochloride (I-72-3) (0.500 g, crude) as white solid. No further purification was performed.

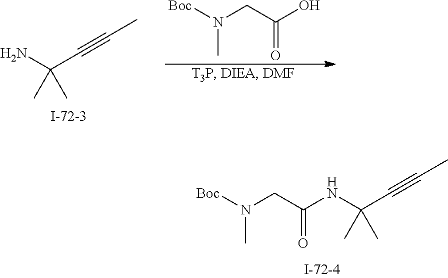

To a solution of 2-((tert-butoxycarbonyl)(methyl)amino)acetic acid (0.420 g, 2.25 mmol) in N,N-dimethylformamide (3 mL) was added N-ethyl-N-isopropylpropan-2-amine (1.45 g, 11.2 mmol), T₃P (2.14 g, 3.37 mmol, 50% w/w in ethyl acetate) and I-72-3 (0.300 g, 2.25 mmol) at 25° C. The mixture was stirred at 25° C. for 3 hours then quenched with water (10 mL) and extracted with ethyl acetate (15 mL*3). The combined organic phases were washed with brine (15 mL*2), dried over anhydrous sodium sulfate, filtered, and filtrate was concentrated under reduced pressure to afford tert-butyl methyl(2-((2-methylpent-3-yn-2-yl)amino)-2-oxoethyl)carbamate (I-72-4) (0.400 g, crude) as brown oil.

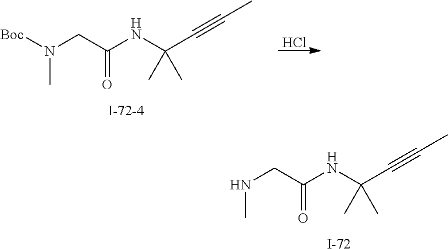

To a solution of I-72-4 (0.400 g, 1.49 mmol) in methanol (3 mL) was added a 4 M solution of hydrochloric acid in methanol (1 mL) at 25° C. After 0.5 hour, the mixture was concentrated under reduced pressure to afford 2-(methylamino)-N-(2-methylpent-3-yn-2-yl)acetamide as a hydrochloride (I-72) (0.240 g, crude) as brown oil.

¹H-NMR of I-72: (CDCl₃, 400 MHz) δ 8.98 (s, 2H), 8.33 (s, 1H), 3.99 (s, 2H), 2.87-2.84 (m, 3H), 1.79 (s, 3H), 1.60 (s, 6H).

### EXAMPLES

### Synthesis of N-(3-chloro-4-methoxyphenyl)-2-((N,5-dimethyl-1H-indazole)-7-sulfonamido)acetamide (Example 1)

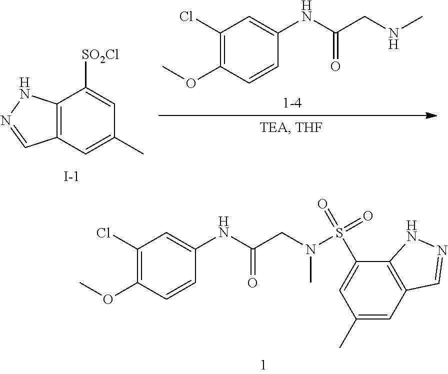

To a mixture of I-1 (0.107 g, 0.405 mmol) and triethylamine (0.205 g, 2.03 mmol) in THF (2 mL) was added a solution of I-4 (0.100 g, 0.405 mmol) in THF (1 mL) drop wise at 0° C. The mixture was stirred at 20° C. for 3 hours then poured into water (100 mL) and extracted with ethyl acetate (50 mL*2). The combined organic layer was washed with brine (50 mL), dried over anhydrous sodium sulfate, filtered, and the filtrate was concentrated under reduced pressure. The residue was purified by prep-HPLC (column: Waters Xbridge C18 150*50 mm*10 um; mobile phase: [water (0.05% am-onia hydroxide v/v)-MeCN]; B %: 32%-62%, 11.5 min) to afford N-(3-chloro-4-methoxyphenyl)-2-(N,5-dimethyl-1H-indazole-7-sulfonamido) acetamide (1) (0.045 g, 26.3% yield) as a white solid.

LCMS of 1: m/z: 423.1 [M+H]⁺

¹H-NMR of 1 (CDCl₃, 400 MHz): δ 11.-0 (br. S, 1H), 8.15-8.14 (m, 2H)-7.86 (s, 1H), 7.64-7.62 (m, 2H), 7.41 (dd, J=8.8, 2.4 Hz, 1H), 6.91 (d, J=8.8 Hz, 1H), 3.95 (s, 3H), 3.89 (s, 2H), 2.91 (s, 3H), 2.56 (s, 3H)

The following compounds were synthesized in a similar manner as example 1.

| | | LCMS | |
|---|---|---|---|
| Example | Reactant | m/z | ¹H-NMR |
| 3 | I-5 | 478.3 | (CDC-₃, 400 MHz): δ 8.20-8.12 |
| | | | (m, 2H), 7.86 (s, 1H), 7.66 (d, J = |
| | | | 2.4 Hz, 1H), 7.65-7.62 (m, 1H), |
| | | | 7.41 (dd, J = 8.8, 2.8 Hz, 1H), 7.03 |
| | | | (d, J- = 8.8 Hz, 1H), 3.96-3.81 (m, |
| | | | 6H), 3.11-2.99 (m, 4H), 2.91 (s, |
| | | | 3H), 2.56 (s, 3H) |
| 6 | I-6 | 390.1 | (DMSO-d₆, 400 MHz) δ 13.16 (br. |
| | | | s, 1H), 10.16 (br. s, 1H), 8.18 (s, |
| | | | 1H)-7.90 (s, 1H), 7.68-7.53 (m, |
| | | | 2H), 6.62 (d, J = 2.0 Hz, 1H), 6.29 |
| | | | (dd, J = 7.2, 2.0 Hz, 1H), 4.11 (s, |
| | | | 2H), 3.33 (s, 3H), 2.83 (s, 3H), 2.46 |
| | | | (s, 3H) |
| 7 | I-7 | 416.1 | (CDCl₃, 400 MHz): δ: 11.32 (br. s, |
| | | | 1H), 8.89 (br. s, 1H), 8.14 (s, 1H), |
| | | | 7.86 (s, 1H), 7.69 (s, 1H), 7.26 (d, |
| | | | J = 6.4 Hz, 1H), 6.78 (s, 1H), 6.65 |
| | | | (s, 1H)-4.35 (s, 1H), 3.60-3.54 (m, |
| | | | 4H), 3.30-3.28 (m, 1H)-2.55 (s, |
| | | | 3H), 2.34-2.32 (m, 1H), 1.87- |
| | | | 1.85 (m, 1H), 1.76-1.75 (m, 2H) |
| 25 | I-11 | 399.0 | (DMSO-d₆, 400 MHz): δ 13.22 (br. |
| | | | s, 1H), 10.31 (br. s, 1H), 8.59 (d, |
| | | | J = 7.2 Hz, 1H), 8.19 (s, 1H), 7.98 |
| | | | (d, J- = 2.0 Hz, 1H), 7.92-7.89 (m, |
| | | | 2H), 7.64 (d, J- = 0.8 Hz, 1H), 6.89- |
| | | | 6.84 (m, 1H), 6.49 (d, J = 2.0 Hz, |
| | | | 1H), 4.14 (s, 2H), 2.86 (s, 3H), 2.45 |
| | | | (s, 3H) |
| 26 | I-12 | 399.1 | (DMSO-d₆, 400 MHz) δ 13.23 (br. |
| | | | s, 1H), 10.66 (br. s, 1H), 8.45 (d, |
| | | | J = 7.2 Hz, 1H), 8.19 (s, 1H), 7.89 (d, |
| | | | J = 11.2 Hz, 2H), 7.82 (s, 1H), 7.65 |
| | | | (s, 1H), 7.45 (s, 1H), 7.00 (d, J = |
| | | | 7.2 Hz, 1H), 4.19 (s, 2H), 2.86 (s, |
| | | | 3H), 2.46 (s, 3H) |
| 29 | I-13 | 416.9 | (DMSO-d₆, 400 MHz): δ 13.27 (br. |
| | | | s, 1H), 9.90 (br. s, 1H), 8.19 (s, |
| | | | 1H), 7.90 (s, 1H), 7.63 (d, J = 0.8 |
| | | | Hz, 1H), 7.13 (d, J = 2.4 Hz, 1H), |
| | | | 6.90 (dd, J = 2.4, 8.8 Hz, 1H), 6.77 |
| | | | (d, J- = 8.8 Hz, 1H), 4.22-4.17 (m, |
| | | | 4H), 4.03 (s, 2H), 2.81 (s, 3H), 2.45 |
| | | | (s, 3H) |
| 98 | I-22 | 416.2 | (DMSO-d₆ + D₂O, 400 MHz) δ 8.16 |
| | | | (s, 1H), 7.89 (s, 1H), 7.59 (s, 1H), |
| | | | 7.45 (d, J = 7.6 Hz, 1H), 6.60 (s, |
| | | | 1H), 6.30 (dd, J = 2.4, 8.0 Hz, 1H)- |
| | | | 4.08 (s, 2H), 3.21-3.19 (m, 1H), |
| | | | 2.80 (s, 3H)-2.42 (s, 3H), 0.95- |
| | | | 0.93 (m, 2H), 0.75-0.73 (m, 2H) |
| 99 | I-23 | 432.2 | (DMSO-d₆, 400 MHz): δ 12.87 (br. |
| | | | s, 1H), 9.96 (br. s, 1H), 8.16 (s, |
| | | | 1H), 7.89 (s, 1H), 7.68 (d, J = 7.2 |
| | | | Hz, 1H)-7.63 (s, 1H), 7.55-7.54 |
| | | | (m, 1H), 6.61 (d, J- = 2.4 Hz, 1H), |
| | | | 6.48-6.43 (m, 1H), 5.49-5.43 (m, |
| | | | 1H), 4.86 (t, J = 7.6 Hz, 2H), 4.70 |
| | | | (t, J- = 6.8 Hz, 2H), 4.54-4.51 (m, |
| | | | 1H), 4.12 (s, 2H)-2.88 (s, 3H), |
| | | | 2.48-2.48 (m, 3H) |
| 100 | I-24 | 460.1 | (CD₃OD, 400 MHz): δ 8.11 (s, 1H), |
| | | | 7.89 (s, 1H), 7.72 (s, 1H), 7.65 (d, |
| | | | J = 8.0 Hz, 1H), 6.83 (d, J = 2.4 |
| | | | Hz, 1H), 6.65 (dd, J = 5.-, 2.4 Hz, |
| | | | 1H), 5.01-4.94 (m, 1H), 4.13 (s, 2H), |
| | | | 4.07 (dd, J = 7-2, 4.0 Hz 2H), 3.60- |
| | | | 3.55 (m, 2H), 2.91 (s, 3H)-2.52 (s, |
| | | | 3H), 1.99-1.89 (m, 2H), 1.81-1.77 |
| | | | (m, 2H) |
| 107 | 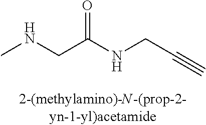 | 321.1 | (CDCl₃, 400 MHz): δ 11.34 (br.s, 1H), 8.13 (s, 1H), 7.84 (s, 1H), 7.61 (d, J = 0.8 Hz, 1H), 6.68 (br.s, 1H), 4.14(dd, J = 2.4 Hz, 4.4 Hz, 2H), 3.79 (s, 2H), 2.83 (s, 3H), 2.55 (s, 3H), 2.29 (t, J = 2.4 Hz, 1H) |
| 108 | 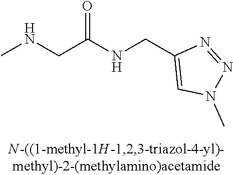 | 378.1 | (DMSO-d₆, 400 MHz): δ 13.41 (br. s, 1H), 8.63 (br. s, 1H), 8.19 (s, 1H), 7.90 (s, 1H), 7.84 (s, 1H), 7.63 (d, J = 0.8 Hz, 1H), 4.30 (d, J = 5.6 Hz, 2H), 4.00 (s, 3H), 3.87 (s, 2H), 2.72 (s, 3H), 2.47 (s, 3H) |
| 109 | I-26 | 387.1 | (CDCl₃, 400 MHz): δ 11.57 (br. s, |
| | | | 1H), 8.10 (s, 1H), 7.82 (s, 1H)-7.62 |
| | | | (s, 1H), 7.26-7.19 (m, 4H), 6.63 (br. |
| | | | s, 1H), 4.51 (d, J = 5.6 Hz, 2H), |
| | | | 3.82 (s, 2H), 2.81 (s, 3H), 2.53 (s, |
| | | | 3H), 2.34 (s, 3H) |
| 110 | I-27 | 387.1 | (CD₃OD, 400 MHz): -8.13 (s, 1H), |
| | | | 7.91-7.89 (m, 1H), 7.73-7.72 (m, |
| | | | 1H), 7.21-7.17 (m, 1H)-7.12 (s, |
| | | | 1H), 7.07-7.06 (m, 2H), 4.36 (s, |
| | | | 2H), 3.93 (s, 2H), 2.81(s, 3H), 2.52 |
| | | | (s, 3H), 2.31(s, 3H) |
| 111 | I-28 | 387.1 | (CDCl₃, 400 MHz): δ 11.44 (br. s, |
| | | | 1H), 8.12 (s, 1H), 7.83 (s, 1H)- |
| | | | 7.62 (s, 1H), 7.23-7.14 (m, 4H), |
| | | | 6.75-6.57 (m, 1H), 4.49 (d, J = 5.6 |
| | | | Hz, 2H), 3.82 (s, 2H), 2.80 (s, 3H), |
| | | | 2.54 (s, 3H), 2.36 (s, 3H) |
| 112 | I-29 | 367.0 | δ 11.70 (br. s, 1H), 8.12 (s, 1H), |
| | | | 7.83 (s, 1H), 7.65 (s, 1H), 6.-9 (br. |
| | | | s, 1H), 4.10-4.01 (m, 1H), 3.87- |
| | | | 3.73 (m, 4H), 3.67-3.61 (m, 1H), |
| | | | 3.57-3.49 (m, 1H), 2.82 (s, 3H)- |
| | | | 2.55 (s, 3H), 2.02-1.64 (m, 4H) |
| 113 | I-30 | 367.3 | (DMSO + D_O, 400 MHz): δ 8.21- |
| | | | 8.09 (m, 1H), 7.94-7.83 (m, 1H), |
| | | | 7.65-7.55 (m, 1H), 3.87-3.79 (m, |
| | | | 2H), 3.68-3.52 (m, 3H), 3.39- |
| | | | -3.28 (m, 1H), 3.15-3.03 (m, 1H), |
| | | | 2.77-2.62 (m, 3H), 2.46-2.31 (m, |
| | | | 3H), 1.82-1.68 (m, 1H), 1.66-1.55 |
| | | | (m, 1H), 1.52-1.32 (m, 2H) |
| 119 | I-31 | 504.1 | (CDCl₃, 400 MHz) δ 11.04 (br. s, |
| | | | 1H), 8.10 (s, 1H), 7.80 (s, 1H), 7.65 |
| | | | (s, 1H), 7.52 (d, J- = 2.4 Hz, 1H), |
| | | | 7.27-7.22 (m, 1H), 6.97 (d, J = 8.4 |
| | | | Hz, 1H), 3.87 (t, J- = 4.8 Hz, 4H), |
| | | | 3.73-3.66 (m, 1H), 3.51-3.39 (m, |
| | | | 3H), 3.05-2.92 (m, 5H), 2.58-2.51 |
| | | | (m, 3H), 2.22-2.09 (m, 2H) |
| 120 | I-32 | 456.1 | (Methanol-d₄, 400 MHz): δ 8.11 (s, |
| | | | 1H), 7.87 (s, 1H)-7.69 (s, 1H), |
| | | | 6.85-6.83 (m, 1H), 6.84-6.79 (m, |
| | | | 2H), 6.60 (d, J = 8.8 Hz, 1H), |
| | | | 4.24(t, J- = 4.4 Hz, 2H), 3.70-3.62 |
| | | | (m, 1H), 3.47-3.39 (m, 2H), 3.38- |
| | | | 3.33 (m, 1H), 3.17(t, J = 4.4 Hz, |
| | | | 2H), 2.96 (t, J = 7.6 Hz, 1H), 2.82 |
| | | | (s, 3H)-2.52 (s, 3H), 2.08-1.98 |
| | | | (m, 2H) |
| 122 | I-33 | 526.2 | (CD₃OD, 400 MHz): δ 8.13(s, 1H-, |
| | | | 7.90(s, 1H), 7.72--7.71(m, 1H), |
| | | | 6.88--6.76(m, 3H), 4.20--4.18(m, |
| | | | 2H), 4.06--4.03(m, 2H), 3.89--3.76 |
| | | | (m, 1H), 3.70--3.59(m, 1H), 3.56- |
| | | | -3.53(m, 2H), 3.52--3.44(m, 3H), |
| | | | 3.37-3.25(m, 2H), 2.98(t, |
| | | | J = 7.6 Hz, 1H-, 2.54(s, 3H), 2.09- |
| | | | -2.00(m, 2H), 1.83--1.77(m, 2H), |
| | | | 1.71-1.67(m, 2H) |
| 123 | I-34 | 498.1 | (DMSO-d₆, 400 MHz): δ 13.16 (br. |
| | | | s, 1H), 9.69 (br. s, 1H), 8.19 (s, |
| | | | 1H), 7.92 (s, 1H), 7.63 (s, 1H), 6.97 |
| | | | (d, J = 2.8 Hz, 1H), 6.82 (dd, J = |
| | | | 8.0, 2.4 Hz, 1H), 6.19 (d, J = 8.4 |
| | | | Hz, 1H), 4.74 (t, J = 6.8 Hz, 2H), |
| | | | 4.63 (t, J- = 6.4 Hz, 2H), 4.48- |
| | | | 4.47 (m, 1H), 4.26 (t, J- = 4.0 Hz, |
| | | | 2H), 3.57-3.53 (m, 1H), 3.32-3.30 |
| | | | (m, 1H), 3.26-3.21 (m, 1H), 3.13 |
| | | | (t, J- = 4.0 Hz, 2H), 2.95-2.91 (m, |
| | | | 1H)-2.47 (s, 3H), 2.01-1.97 (m, |
| | | | 1H), 1.86-1.81 (m, 1H) |
| 126 | I-5 | 478.3 | (CDC-₃, 400 MHz): δ 8.20-8.12 |
| | | | (m, 2H), 7.86 (s, 1H), 7.66 (d, J- = |
| | | | 2.4 Hz, 1H), 7.65-7.62 (m, 1H), |
| | | | 7.41 (dd, J = 8.8, 2.8 Hz, 1H), 7.03 |
| | | | (d, J- = 8.8 Hz, 1H), 3.96-3.81 |
| | | | (m, 6H), 3.11-2.99 (m, 4H), 2.91 |
| | | | (s, 3H), 2.56 (s, 3H) |
| 163 | I-42 | 500.1 | (400 MHz, chloroform-d) δ 11.30 |
| | | | (br. s, 1H), 8.14 (s, 1H), 8.01 (br s, |
| | | | 1H), 7.86 (s, 1H), 7.64 (s, 1H), 7.19 |
| | | | (d, J = 2.0 Hz, 1H), 6.76 (d, J = 8.4 |
| | | | Hz, 1H), 6.64 (dd, J = 8.-, 2.4 Hz, |
| | | | 1H), 4.22-4.20 (m, 2H), 4.10 (dd, |
| | | | J = 10.-, 3.6 Hz, 2H), 3.90-3.82 |
| | | | (m, 3H), 3.59 (dt, J = 11.-, 2.4 Hz, |
| | | | 2H), 3.32-3.29 (m, 2H), 2.89 (s, |
| | | | 3H)-2.56 (s, 3H), 1.88-1.75 (m, 4H) |
| 169 | I-45 | 472.2 | (CD-l₃, 400 MHz) δ 8.18-8.10 (m, |
| | | | 2H), 7.85 (s, 1H), 7.65 (s, 1H), 6.86 |
| | | | (d, J = 2.0 Hz, 1H), 6.75 (d, J = 8.4 |
| | | | Hz, 1H), 6.65 (dd, J = 8.4, 2.0 Hz, |
| | | | 1H), 4.91 (t, J = 6.8 Hz, 2H), 4.82 |
| | | | (t, J- = 6.8 Hz, 2H), 4.68-4.62 (m, |
| | | | 1H), 4.39-4.32 (m, 2H)-3.87 (s, |
| | | | 2H), 3.30-3.23 (m, 2H), 2.89 (s, |
| | | | 3H), 2.55 (s, 3H) |

### Synthesis of N-(3-chloro-4-methoxyphenyl)-2-((N,5-dimethyl-2-oxoindoline)-7-sulfonamido)acetamide (Example 2)

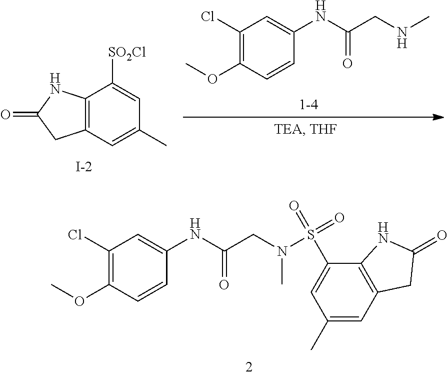

To a mixture of I-2 (0.032 g, 0.12 mmol) and triethylamine (0.062 g, 0.61 mmol) in THF (1.5 mL) was added a solution of I-4 (0.030 g, 0.12 mmol) in THF (1 mL) drop wise at 0° C. The mixture was stirred at 20° C. for 3 hours. The mixture was concentrated under reduced pressure. The residue was purified by prep-HPLC (column: Waters Xbridge 150*25 mm*5 um; mobile phase: [water (0.05% ammonia hydroxide v/v)-MeCN]; B %: 27%-57%, 10 min) to afford N-(3-chloro-4-methoxyphenyl)-2-(N,5-dimethyl-2-oxoindoline-7-sulfonamido)acetamide (2) (0.025 g, 18% yield) as a white solid.

LCMS of 2: m/z: 438.0 [M+H]⁺

¹H-NMR of 2 (CDCl₃, 400 MHz): δ 8.83 (s, 1H), 8.20 (s, 1H), 7.64 (d, J=2.4 Hz, 1H), 7.41 (dd, J=8.8, 2.4 Hz, 1H), 7.29 (s, 2H), 6.90 (d, J=8.8 Hz, 1H), 3.90 (s, 3H), 3.84 (s, 2H), 3.55 (s, 2H), 2.92 (s, 3H), 2.39 (s, 3H)

### Synthesis of (S)—N-(3-chloro-4-methoxyphenyl)-1-((5-methyl-1H-indazol-7-yl)sulfonyl)pyrrolidine-2-carboxamide (Example 4)

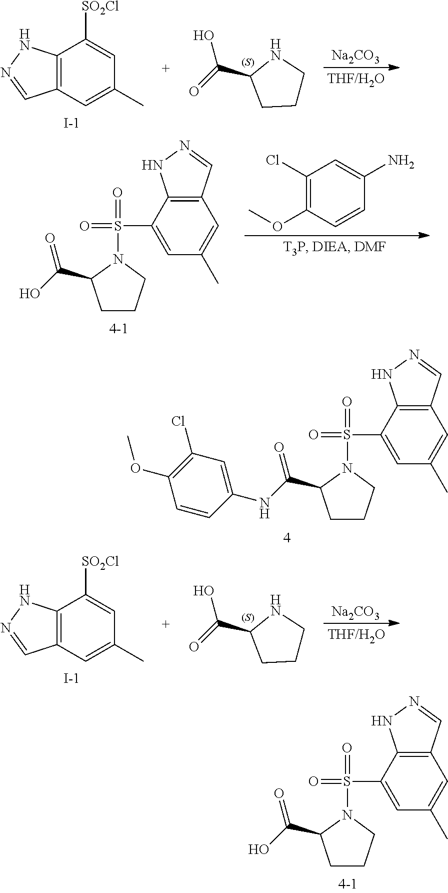

Sodium carbonate (0.055 g, 0.52 mmol) was added to a solution of (S)-proline (0.066 g, 0.57 mmol) in a 1:1 mixture of water:THF (3.0 mL) with continuous stirring until the solid was dissolved. The solution was cooled to −5° C. and I-1 (0.120 g, 0.520 mmol) was added in four portions over a period of 1 hour. The slurry was further stirred at 20° C. for 4 hours. The mixture was acidified to approximately pH=2 with 1 N aqueous hydrochloric acid, then extracted with ethyl acetate (20 mL*3). The combined organic layers were washed with brine (50 mL), dried over anhydrous sodium sulfate, filtered, and the filtrate was concentrated under reduced pressure to afford (S)-1-((5-methyl-1H-indazol-7-yl)sulfonyl)pyrrolidine-2-carboxylic acid (4-1) (0.110 g, 57.7% yield) as a yellow solid.

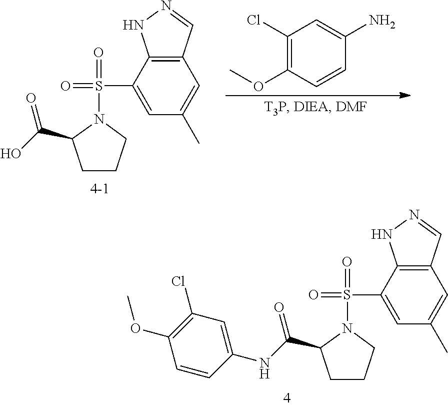

To a solution of 4-1 (0.100 g, 0.323 mmol) and 3-chloro-4-methoxyaniline (0.056 g, 0.36 mmol) in N,N-dimethylformamide (2 mL) was added T₃P (0.290 mL, 0.485 mmol, 50% solution in ethyl acetate) and diisopropylethylamine (0.167 g, 1.29 mmol) at 0° C. The mixture was stirred at 0° C. for 2 hours then poured into water (50 mL) and extracted with ethyl acetate (20 mL*3). The combined organic layers were washed with brine (100 mL), dried over anhydrous sodium sulfate, filtered, and the filtrate was concentrated under reduced pressure. The residue was purified by prep-HPLC (column: Waters Xbridge 150*25 mm*5 um; mobile phase: [water (0.05% ammonia hydroxide v/v)-ACN]; B %: 32%-62%, 10 min) and the eluent concentrated under reduced pressure to afford (S)—N-(3-chloro-4-methoxyphenyl)-1-((5-methyl-1H-indazol-7-yl)sulfonyl)pyrrolidine-2-carboxamide (4) (0.036 g, 18.6% yield) as a white solid.

LCMS of 4: m/z 449.1 [M+H]⁺

¹H-NMR of 4 (CDCl₃, 400 MHz): δ 11.33 (br. s, 1H), 8.44 (br. s, 1H), 8.14 (s, 1H), 7.85 (s, 1H), 7.70 (d, J=0.8 Hz, 1H), 7.66 (d, J=2.4 Hz, 1H), 7.42 (dd, J=8.8, 2.8 Hz, 1H), 6.90 (d, J-=8.8 Hz, 1H), 4.38-4.32 (m, 1H)-3.91 (s, 3H), 3.64-3.57 (m, 1H), 3.36-3.28 (m, 1H)-2.55 (s, 3H), 2.38-2.29 (m, 1H), 1.93-1.82 (m, 1H), 1.82-1.72 (m, 2H).

The following compound was synthesized in a similar manner as 4 using the reactant indicated.

| Example | Reactant | LCMS m/z | ¹H-NMR |
|---|---|---|---|
| 5 | 3-chloro-4- | 504.2 | (CDCl₃, 400 MHz): δ 11.26 (br. s, 1H), 8.47 |
| | morpholinoaniline | | (s, 1H), 8.14 (s, 1H) −7.86 (s, 1H), 7.71- |
| | | | 7.69 (m, 2H), 7.44 (dd, J = 8.8, 2.4 Hz, 1H), |
| | | | 7.03 (d, J = 8.8 Hz, 1H), 4.34 (dd, J = 8.8, 2.4 |
| | | | Hz, 1H), 3.89 (t, J −= 4.4 Hz, 4H), 3.66-3.58 |
| | | | (m, 1H), 3.34-3.26 (m, 1H), 3.04 (t, J = 4.8 |
| | | | Hz, 4H) −2.55 (s, 3H), 2.38-2.29 (m, 1H), |
| | | | 1.92-1.70 (m, 3H) |

### Synthesis of N-(3-chloro-4-methoxyphenyl)-2-((5-(difluoromethyl)-N-methyl-1H-indazole)-7-sulfonamido)acetamide (Example 8)

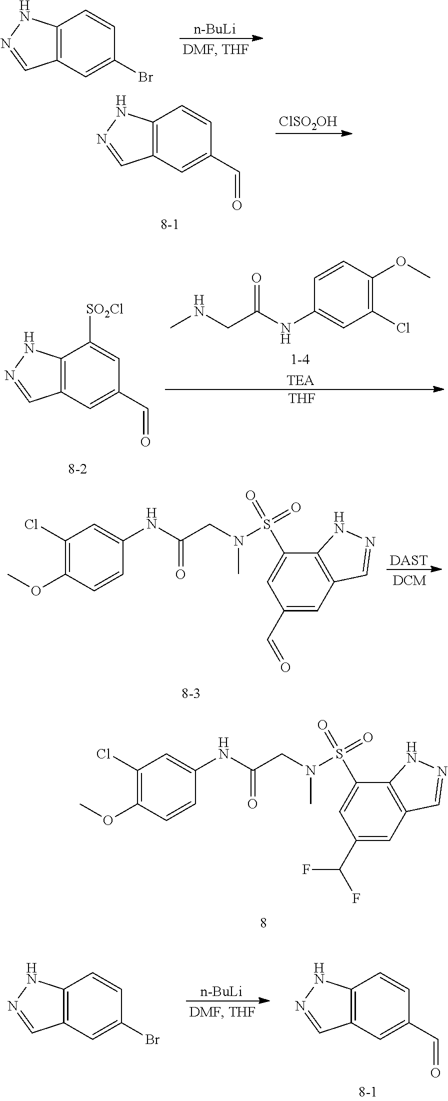

To a solution of 5-bromo-1H-indazole (3.00 g, 15.2 mmol) in THF (40 mL) was added n-BuLi (18.3 mL, 45.7 mmol, 2.5 M in hexane) at −60° C. After 2 hours, N, N-dimethylformamide (2.34 mL, 30.5 mmol) was added under nitrogen atmosphere. The mixture was stirred at −60° C.-20° C. for 12 hours. The reaction was diluted with water (100 mL) and extracted with ethyl acetate (50 mL*2). The combined organic layers were washed with brine (100 mL), dried over anhydrous sodium sulfate, filtered, and the filtrate was concentrated under reduced pressure. The residue was purified by column chromatography (SiO₂, petroleum ether/ethyl acetate=15/1˜8/1) to afford 1H-indazole-5-carbaldehyde (8-1) (1.00 g, 43.3% yield) as a white solid.

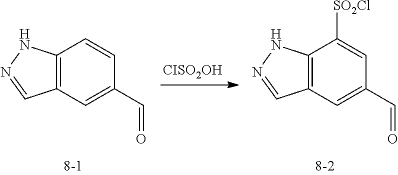

A round bottom flask was charged with chlorosulfonic acid (10.0 mL, 150 mmol) and cooled to 0° C. To this 8-1 (0.600 g, 4.11 mmol) was added slowly at 0° C. The mixture was heated to 100° C. and stirred for 12 hours. After being cooled to room temperature, the mixture was slowly poured into ice-water (100 mL) and extracted with ethyl acetate (50 mL). The organic layer was washed with brine (100 mL), dried over anhydrous sodium sulfate, filtered, and the filtrate was concentrated under reduced pressure. The residue was purified by column chromatography (SiO₂, petroleum ether/ethyl acetate=10/1˜ 8/1) to afford 5-formyl-1H-indazole-7-sulfonylchloride (8-2) (0.200 g, 15.3% yield) as a red solid.

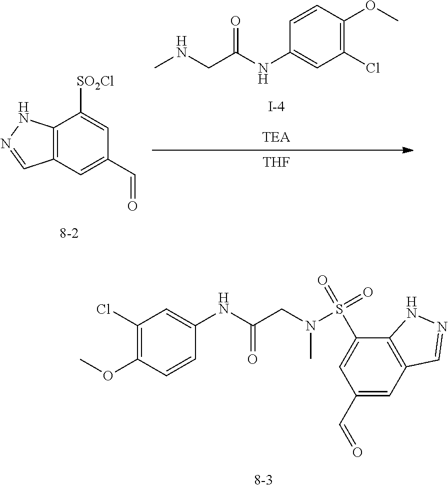

To a mixture of 8-2 (0.200 g, 0.628 mmol) and I-4 (0.144 g, 0.629 mmol) in THF (4 mL) was added triethylamine (0.263 mL, 1.89 mmol) at 25° C. The mixture was stirred at 25° C. for 1 hour then diluted with water (50 mL) and extracted with ethyl acetate (20 mL*2). The combined organic layers were washed with brine (50 mL), dried over anhydrous sodium sulfate, filtered, and the filtrate was concentrated under reduced pressure. The residue was purified by column chromatography (SiO₂, petroleum ether/ethyl acetate=5/1˜1/1) to afford N-(3-chloro-4-methoxyphenyl)-2-(5-formyl-N-methyl-1H-indazole-7-sulfonamido)acetamide (8-3) (0.150 g, 43.0% yield) as a yellow solid.

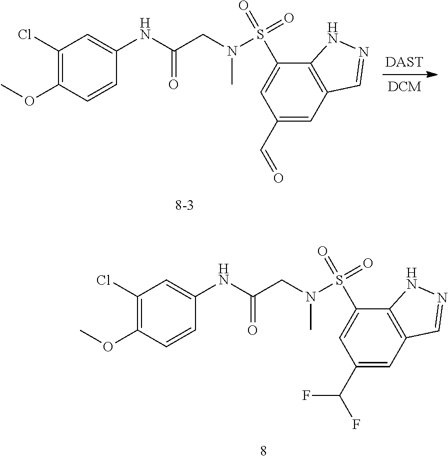

To a solution of 8-3 (0.080 g, 0.144 mmol) in dichloromethane (3 mL) was added DAST (0.152 mL, 1.15 mmol) at 0° C. The mixture was stirred at 0-25° C. for 4 hours then diluted with a saturated aqueous solution of sodium bicarbonate (30 mL) and extracted with ethyl acetate (10 mL*2). The combined organic layers were washed with brine (30 mL), dried over anhydrous sodium sulfate, filtered, and the filtrate was concentrated under reduced pressure. The residue was purified by prep-TLC (SiO₂, petroleum ether/ethyl acetate=1/2), followed by prep-HPLC (column: Waters Xbridge 150*25 mm*5 um; mobile phase: [water (0.05% ammonia hydroxide v/v)-MeCN]; B %: 35%-65%, 10 min) to afford N-(3-chloro-4-methoxyphenyl)-2-(5-(difluoromethyl)-N-methyl-1H-indazole-7-sulfonamido)acetamide (8) (0.007 g, 10.1% yield) as a white solid.

LCMS of 8: m/z 459.1 [M+H]⁺

¹H-NMR of 8 (CD₃OD, 400 MHz): δ 8.34 (s, 2H), 8.04 (s, 1H), 7.57 (d, J=2.8 Hz, 1H), 7.33 (dd, J=2.8, 8.8 Hz, 1H), 7.11-6.83 (m, 2H), 4.16 (s, 2H), 3.86 (s, 3H), 2.93 (s, 3H)

The following compound was synthesized in a similar manner as 8 using the reactant indicated.

| | | LCMS | |
|---|---|---|---|
| Example | Reactant | m/z | ¹H-NMR |
| 10 | 1-5 | 514.0 | (CD₃OD, 400 MHZ): δ 8.34 |
| | | | (s, 2H), 8.04 (s, 1H), |
| | | | 7.62 (d, J = 2.4 Hz, 1H), |
| | | | 7.34 (dd, J = 8 .−, 2.4 Hz, |
| | | | 1H), 7.13-6.81 (m, 2H) |
| | | | −4.18 (s, 2H), 3.86-3.81 |
| | | | (m, 4H), 3.02-2.97 (m, 4H), |
| | | | 2.93 (s, 3H) |
| 37 | I-6 | 426.0 | (CD₃OD, 400 MHZ): δ 8.32 |
| | | | (s, 2H), 8.01 (s, 1H), |
| | | | 7.54 (d, J = 7.2 Hz, 1H), |
| | | | 6.97 (t, J = 56 Hz, 1H), |
| | | | 6.77 (d, J = 2.4 Hz, 1H), |
| | | | 6.55 (dd, J₁ = 2.4 Hz, J₂ = |
| | | | 7.6 Hz, 1H), 4.21 (s, 2H), |
| | | | 3.49 (s, 3H), 2.95 (s,3H) |

### Synthesis of N-(3-chloro-4-methoxyphenyl)-2-((N,3,5-trimethyl-1H-indazole)-7-sulfonamido)acetamide (Example 9)

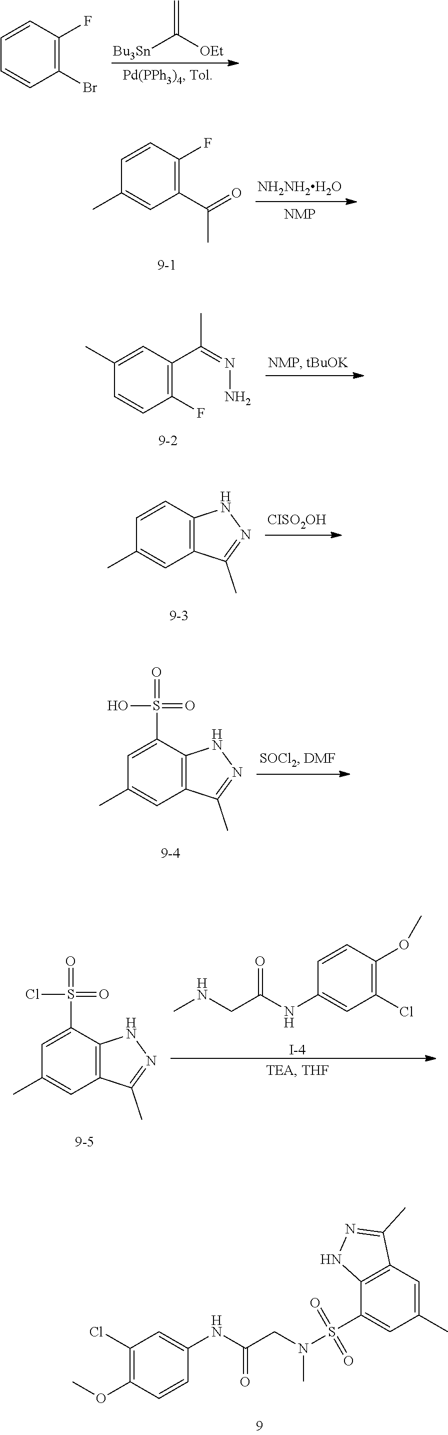

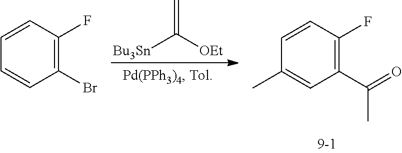

The mixture of 2-bromo-1-fluoro-4-methylbenzene (2.50 g, 13.2 mmol), tributyl (1-ethoxyvinyl)stannane (4.67 mL, 13.8 mmol) and Pd(PPh₃)₄ (0.764 g, 0.661 mmol) in toluene (25 mL) was stirred at 120° C. for 2 hours under nitrogen. To the mixture was added a saturated solution of potassium fluoride (20 mL) and ethyl acetate (200 mL). The mixture was stirred at 20° C. for 0.5 hour during which time the mixture turned into an orange suspension. The mixture was filtered, the organic phase was separated and washed with an aqueous solution of hydrochloric acid (5 M, 10 mL) followed by brine (20 mL), then dried over anhydrous sodium sulfate, filtered, and the filtrate was concentrated under reduced pressure. The residue was purified by silica gel chromatography (petroleum ether/ethyl acetate=1:0), followed by reversed phase HPLC (0.1% TFA condition) to afford 1-(2-fluoro-5-methylphenyl)ethan-1-one (9-1) (1.80 g, 89.4% yield) as colorless oil.

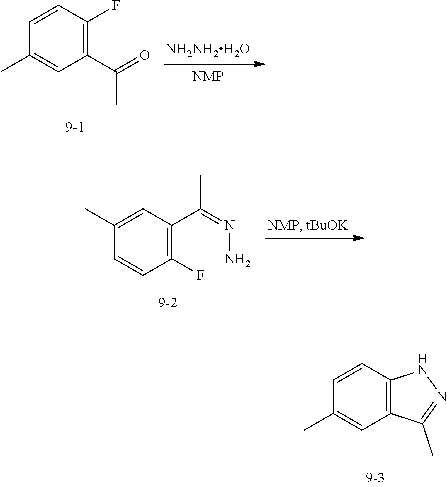

To a stirred solution of 9-1 (0.200 g, 1.31 mmol) in n-methyl-pyrrolidone (12 mL) was added hydrazine hydrate (0.130 mL 2.63 mmol) in one portion at 20° C. under a nitrogen atmosphere. The mixture was stirred at 20° C. for 1 hour then placed into a microwave tube, and potassium tert-butoxide (0.525 g, 1.56 mmol) was added. The sealed tube was heated at 200° C. for 1 hour in a microwave reactor. The cooled mixture was diluted with water (100 mL) and extracted with ethyl acetate (250 mL*3). The combined organic phase was washed with brine (150 mL*3), dried over anhydrous sodium sulfate, filtered, and the filtrated was concentrated under reduced pressure. The residue was purified by reversed phase HPLC (0.1% TFA condition), followed by prep-TLC (petroleum ether:ethyl acetate=5:1) to afford 3, 5-dimethyl-1H-indazole (9-3) (0.070 g, 41% yield) as a white solid.

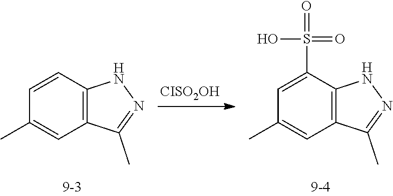

A round bottom flask was charged with chlorosulfonic acid (2 mL) and cooled to 0° C. To this was added 9-3 (0.090 g, 0.62 mmol) slowly at 0° C. The mixture was stirred for 0.5 hour at 0° C. The mixture was added into ice-water (v:v=1:1, 10 mL) drop wise, causing a solid to precipitate out. The formed solid was collected by filtration and the filter cake was triturated with acetonitrile (2 mL) to afford 3,5-dimethyl-1H-indazole-7-sulfonic acid (9-4) (0.090 g, 65% yield) as a white solid.

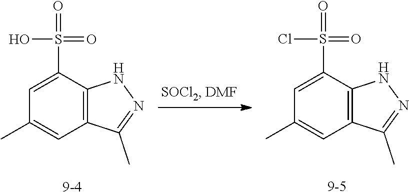

To a solution of 9-4 (0.070 g, 0.31 mmol) in thionyl chloride (1.75 mL, 24.1 mmol) was added dimethylformamide (0.002 g, 0.031 mmol). The mixture was stirred for 1 hour at 80° C. then concentrated under reduced pressure. The residue was purified by prep-TLC (SiO₂, petroleum ether:ethyl acetate=5:1) to afford 3, 5-dimethyl-1H-indazole-7-sulfonyl chloride (9-5) (0.035 g, 44% yield) as a white solid.

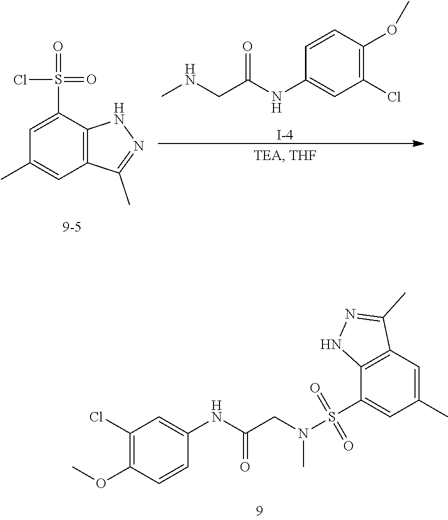

To a solution of I-4 (0.054 g, 0.20 mmol) and triethylamine (0.142 mL, 1.02 mmol) in tetrahydrofuran (0.5 mL) was added 9-5 (0.050 g, 0.20 mmol) in tetrahydrofuran (0.5 mL) at 20° C. The mixture was stirred for 1 hour at 20° C. The mixture was diluted with water (5 mL) and extracted with ethyl acetate (20 mL*3). The combined organic phase was washed with brine (15 mL), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH₄HCO₃)-MeCN]; B %: 30%-60%, 10 min) to afford N-(3-chloro-4-methoxyphenyl)-2-(N-3,5-trimethyl-1H-indazole-7-sulfonamido)acetamide (9) (0.035 g, 39% yield) as a white solid.

LCMS of 9: m/z 437.0 [M+H]⁺

¹H-NMR of 9: (CDCl₃, 400 MHz): δ 10.85 (Br. s, 1H), 8.14 (br. s, 1H)-7.76 (s, 1H), 7.61-7.60 (m, 2H), 7.40 (dd, J=8.8, 2.8 Hz, 1H), 6.90 (d, J=8.8 Hz, 1H), 3.90 (s, 3H), 3.84 (s, 2H), 2.90 (s, 3H), 2.61 (s, 3H), 2.55 (s, 3H)

The following compounds were synthesized in a similar manner as 9 using the reactant indicated.

| Ex- | Re- | LCMS | |
|---|---|---|---|
| ample | actant | m/z | ¹H-NMR |
| 36 | I-6 | 404.0 | (DMSO-d₆, 400 MHZ) δ 12.75 (br. s, 1H), 10.13 |
| | | | (br. s, 1H) −7.85 (s, 1H), 7.59-7.57 (m, 2H) −6.62 |
| | | | (s, 1H), 6.30-6.28 (m, 1H), 4.07 (s, 2H), 3.32 (s, |
| | | | 3H), 2.81 (s, 3H), 2.50 (s, 3H), 2.46 (s, 3H) |
| 92 | I-16 | 444.1 | (CD₃OD, 400 MHZ) δ 7.84 (s, 1H) −7.72 (s, 1H), |
| | | | 6.93-6.87 (m, 2H), 6.64 (d, J = 8.8 Hz, 1H), 4.26 |
| | | | (t, J = 4.8 Hz, 2H), 4.03 (s, 2H), 3.22 (m, J = 4.4 |
| | | | Hz, 2H), 2.86 (s, 3H), 2.84 (s, 3H), 2.57 (s, 3H), |
| | | | 2.52 (s, 3H) |

### Synthesis of 2-((N,5-dimethyl-1H-indazole)-7-sulfonamido)-N-(3-methyl-4-morpholinophenyl)acetamide (Example 11)

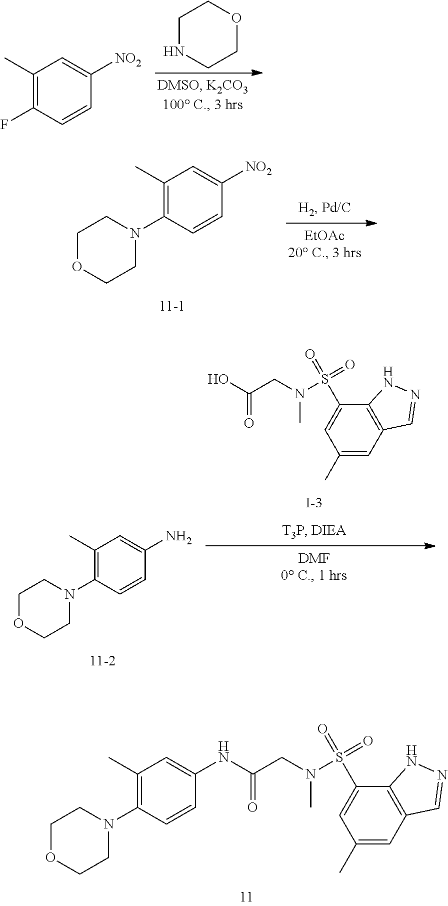

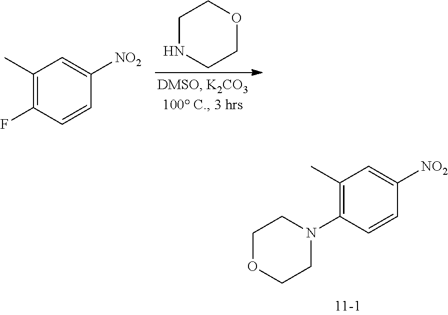 [SMILES: Cc1cc([N+](=O)[O-])ccc1F ; C1COCCN1 ; Cc1cc([N+](=O)[O-])ccc1N1CCOCC1]

A mixture of 1-fluoro-2-methyl-4-nitrobenzene (1.00 g, 6.45 mmol), sodium carbonate (2.67 g, 19.3 mmol) and morpholine (0.850 g, 9.76 mmol) in dimethylsulfoxide (10 mL) was stirred for 3 hours at 100° C. After cooling to room temperature, the mixture was poured into water (50 mL) and extracted with ethyl acetate (50 mL*2). The combined organic phase was washed with brine (50 mL*3), dried over anhydrous sodium sulfate, filtered, and the filtrated was concentrated under reduced pressure to afford 4-(2-methyl-4-nitrophenyl)morpholine (11-1) (1.10 g, 70.5% yield) as a yellow solid. No further purification was performed.

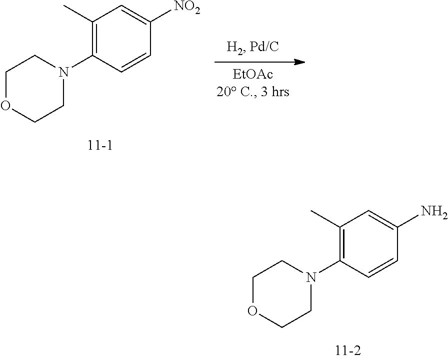 [SMILES: Cc1cc([N+](=O)[O-])ccc1N1CCOCC1 ; Cc1cc(N)ccc1N1CCOCC1]

A solution of 11-1 (1.10 g, 4.95 mmol) in ethyl acetate (15 mL) was degassed with nitrogen three times, then Pd/C (0.200 g, 5% purity on charcoal, wet) was added in one portion. The mixture was degassed with hydrogen three times and stirred for 2 hours at 20° C. under a hydrogen atmosphere (15 PSI). The mixture was filtered and the filtrate was concentrated under reduced pressure to afford 3-methyl-4-morpholinoaniline (11-2) (1 g, crude) as red solid and used directly without further purification.

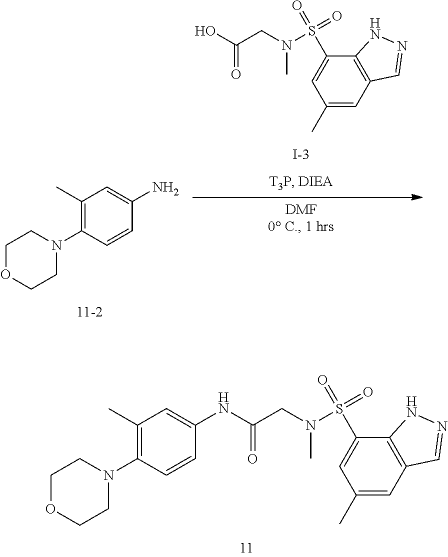

To a mixture of 11-2 (0.080 g, 0.42 mmol), I-3 (0.120 g, 0.424 mmol), and diisopropylethylamine (0.300 mL, 1.72 mmol) in dimethyl formamide (1 mL) was added T₃P (0.400 mL, 0.673 mmol, 50% in ethyl acetate) at 0° C. The mixture was stirred for 3 hours at 50° C. then poured into water (50 mL) and extracted with ethyl acetate (50 mL*2). The combined organic phase was washed with brine (50 mL), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH4HCO3)-ACN]; B %: 33%-63%, 10 min) to afford (2-(N,5-dimethyl-1H-indazole-7-sulfonamido)-N-(3-methyl-4-morpholinophenyl)acetamide) (11) (0.051 g, 26.7% yield) as a white solid.

LCMS of 11: m/z 458.1 [M+H]⁺

¹H-NMR of 11: (CDCl₃, 400 MHz): δ=11.41 (br. s, 1H), 8.14 (s, 2H), 7.85 (s, 1H)-7.64 (s, 1H), 7.43-7.33 (m, 2H), 7.00 (d, J-=8.4 Hz, 1H), 3.91-3.83 (m, 6H), 2.93-2.84 (m, 7H), 2.56 (s, 3H), 2.32 (s, 3H)

The following compounds were synthesized in a similar manner as 11 replacing 11-2 with the reactant listed below.

| | | LCMS | |
|---|---|---|---|
| Example | Reactant | m/z | ¹H-NMR |
| 12 | I-8 | 472.2 | (CDCl₃, 400 MHz) δ 11.23 (br. s, 1H), |
| | | | 8.14 (s, 1H), 8.07 (s, 1H), 7.86 (s, 1H), |
| | | | 7.64 (s, 1H), 7.43 (dd, J = 8.8, 2.8 Hz, |
| | | | 1H), 7.35 (d, J = 2.8 Hz, 1H), 7.08 (d, |
| | | | J- = 8.8 Hz, 1H), 3.87-3.84 (m, 6H), |
| | | | 2.91-2.87 (m, 7H), 2.73 (q, J = 7.6 Hz, |
| | | | 2H), 2.56 (s, 3H), 1.27 (t, J = 7.6 Hz, |
| | | | 3H) |
| 13 | I-9 | 484.2 | (CDCl₃, 400 MHz) δ 11.24 (br. s, 1H), |
| | | | 8.14 (s, 1H), 8.03 (s, 1H), 7.86 (s, 1H), |
| | | | 7.63 (s, 1H), 7.38 (dd, J = 8.8, 2.4 Hz, |
| | | | 1H), 7.01 (d, J = 8.4 Hz, 1H), 6.88 (d, |
| | | | J- = 2.4 Hz, 1H), 3.89-3.87 (m, 4H)− |
| | | | 3.85 (s, 2H), 3.03-3.01 (m, 4H), 2.89 |
| | | | (s, 3H)-2.56 (s, 3H), 2.37-2.30 (m, 1H), |
| | | | 1.05-1.00 (m, 2H), 0.75-0.74 (m, 2H) |
| 146 | I-39 | 488.3 | (DMSO-d₆, 400 MHz) δ 9.82 (s, 1H), |
| | | | 8.19 (s, 1H), 7.90 (s, 1H), 7.63 (d, J = |
| | | | 0.8 Hz, 1H), 6.95 (d, J = 2.4 Hz, 1H), |
| | | | 6.85 (dd, J = 8.8, 2.4 Hz, 1H), 6.73 (d, |
| | | | J- = 8.8 Hz, 1H), 4.13-4.08 (m, 2H)- |
| | | | 4.01 (s, 2H), 3.44-3.36 (m, 2H), 3.09 |
| | | | (s, 2H), 2.78 (s, 3H), 2.46 (s, 3H), 1.13 |
| | | | (s, 6H) |
| 157 | 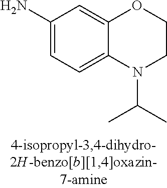 | 458.2 | (400 MHz, METHANOL-d4) δ 9.78 (s, 1H), 8.20 (s, 1H), 7.91 (s, 1H), 7.64 (d, J = 1.1 Hz, 1H), 6.96 (d, J = 2.3 Hz, 1H), 6.91 (dd, J = 2.3, 8.9 Hz, 1H), 6.74 (d, J- = 9.0 Hz, 1H), 4.21- 4.12 (m, 2H)-4.02 (s, 3H), 3.20-3.10 (m, 2H), 2.79 (s, 3H), 2.46 (s, 3H), 1.09 (d, J = 6.5 Hz, 6H) |
| 158 | 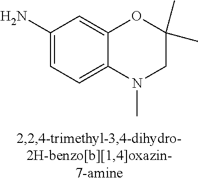 | 458.0 | (400 MHz, ACETONITRILE-d3) δ 8.42 (br s, 1H), 8.13 (s, 1H), 7.92 (s, 1H)-7.77 (s, 1H), 7.03-6.89 (m, 2H), 6.69 (d, J = 9.1 Hz, 1H), 4.05 (s, 2H), 2.97 (s, 2H), 2.89 (s, 3H), 2.80 (s, 3H), 2.53 (s, 3H), 1.32 (s, 6H) |
| 159 | 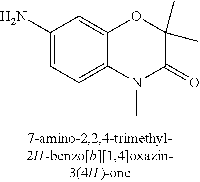 | 472.1 | (400 MHz, DMSO-d6) δ 13.28 (br s, 1H), 10.10 (s, 1H), 8.21 (s, 1H), 7.92 (s, 1H), 7.65 (d, J = 1.0 Hz, 1H), 7.30 (d, J- = 2.1 Hz, 1H), 7.19-7.12 (m, 1H), 7.12-7.05 (m, 1H), 4.09 (s, 2H), 3.27 (s, 3H), 2.84 (s, 3H), 2.46 (s, 3H), 1.40 (s, 6H) |
| 203 | 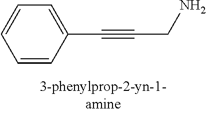 | 397.1 | (DMSO-d6, 400 MHz) δ 13.32 (br. s, 1H), 8.64 (t, J = 5.2 Hz, 1H), 8.19 (s, 1H), 7.91 (s, 1H), 7.64 (d, J- = 1.2 Hz, 1H), 7.43-7.40 (m, 2H), 7.39-7.36 (m, 3H), 4.14 (d, J = 5.2 Hz, 2H), 3.90 (s, 2H), 2.74 (s, 3H), 2.48 (s, 3H) |
| 204 | 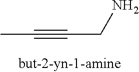 | 335.0 | (CD₃OD, 400 MHz) δ-8.13 (s, 1H), 7.91-7.89 (m, 1H), 7.72 (d, J = 1.2 Hz, 1H), 3.91 (q, J = 2.4 Hz, 2H), 3.89 (s, 2H), 2.80 (s, 3H), 2.53 (s, 3H), 1.78 (t, J = 2.4 Hz, 3H) |

The following compounds were synthesized in a similar manner as shown from 11-2 to 11 using the amide coupling conditions outlined in the General Experimental Method A section above.

| | | | LCMS | |
|---|---|---|---|---|
| Example | Reactant | Method | m/z | ¹H-NMR |
| 48 | 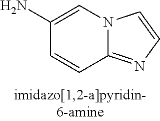 | 1 | 399.1 | (400 MHz, DMSO-d6) δ 13.19 (s, 1H), 10.15 (s, 1H), 9.04 (s, 1H), 8.16 (s, 1H), 7.96 (br s, 1H), 7.87 (s, 1H), 7.61 (s, 1H), 7.50 (br d, J = 9.0 Hz, 2H), 7.09 (br d, J = 9.2 Hz, 1H), 4.10 (s, 2H), 2.83 (s, 3H), 2.41 (s, 3H) |
| 49 | 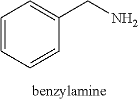 | 1 | 373.2 | (400 MHz, METHANOL-d4) δ 8.15 (s, 1H), 7.92 (s, 1H)-7.75 (s, 1H), 7.37-7.25 (m, 5H), 5.51 (s, 1H), 4.89 (s, 20H), 4.42 (s, 2H)-3.95 (s, 2H), 3.36-3.30 (m, 7H), 2.84 (s, 3H), 2.55 (s, 3H) |
| 50 | 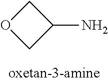 | 2 | 339.1 | (400 MHz, METHANOL-d4) δ 8.14 (s, 1H), 7.92 (s, 1H)-7.74 (s, 1H), 4.96-4.89 (m, 1H), 4.86-4.83 (m, 2H), 4.57 (t, J = 6.4 Hz, 2H), 3.95 (s, 2H), 2.84 |
| | | | | (s, 3H), 2.55 (s, 3H) |
| 51 | 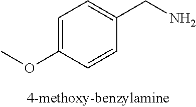 | 2 | 403.2 | (400 MHz, METHANOL-d4) δ 8.15 (s, 1H), 7.92 (s, 1H), 7.74 (s, 1H), 7.23 (d, J = 8.5 Hz, 2H), 6.89 (d, J = 8.7 Hz, 2H), 4.34 (s, 2H), 3.93 (s, 2H), 3.79 (s, 3H), 2.82 (s, 3H)148- ydroxyams, 3H) |
| 52 | 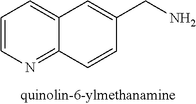 [SMILES: NCc1ccc2ncccc2c1] | 1 | 410.1 | (400 MHz, METHANOL-d4) δ 8.78 (dd, J = 1.-, 4.3 Hz, 1H), 8.35-8.29 (m, 2H), 8.15 (s, 1H), 8.00 (d, J = 9.1 Hz, 1H), 7.93 (s, 1H), 7.82-7.77 (m, 2H), 7.54 (dd, J = 4.3, 8.3 Hz, 1H), 4.23 (s, 2H), 2.96 (s, 3H), 2.53 |
| | | | | (s, 3H) |
| 53 | 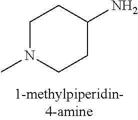 | 2 | 380.2 | (400 MHz, METHANOL-d4) δ 8.14 (s, 1H), 7.91 (s, 1H), 7.74 (d, J = 0.7 Hz, 1H)-3.92 (s, 2H), 3.73-3.63 (m, 1H), 2.90-2.79 (m, 5H), 2.54 (s, 3H), 2.29 (s, 3H), 2.14 (br t, J-11.1 Hz, 2H), 1.91-1.80 (m, 2H), 1.62-1.47 (m, 2H) |
| 54 | 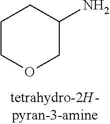 | 2 | 367.1 | (400 MHz, METHANOL-d4) δ 8.10 (s, 1H), 7.88 (dd, J = 0.9, 1.5 Hz, 1H), 7.70 (d, J = 1.1 Hz, 1H)-3.90 (s, 2H), 3.82- 3.67 (m, 3H), 3.48 (ddd, J = 3.1, 8.6-11.6 Hz, 1H), 3.28- 3.22 (m, 1H), 2.79 (s, 3H)- 2.51 (s, 3H), 1.92-1.83 (m, |
| | | | | 1H), 1.77-1.69 (m, 1H), 1.62- |
| | | | | 1.50 (m, 2H) |
| 55 | 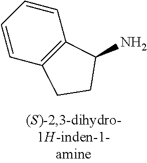 | 2 | 399.2 | (400 MHz, METHANOL-d4) δ = 8.10 (s, 1H), 7.87 (s, 1H), 7.72 (d, J- = 0.9 Hz, 1H), 7.22- 7.14 (m, 4H), 5.36 (t, J = 7.7 Hz, 1H), 3.96 (d, J- = 3.1 Hz, 2H), 3.02-2.93 (m, 1H), 2.88- 2.83 (m, 1H), 2.82 (s, 3H)-2.51 (s, 3H), 2.49-2.41 (m, 1H), 1.83 (qd, J = 8.2, 12.7 Hz, 1H) |
| 56 | 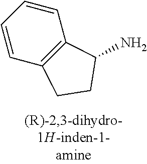 | 2 | 399.2 | (400 MHz, METHANOL-d4) δ 8.15 (s, 1H), 7.92 (s, 1H)- 7.76 (s, 1H), 7.28-7.19 (m, 4H), 5.40 (t, J = 7.6 Hz, 1H), 4.00 (d, J- = 3.3 Hz, 2H), 3.07- 2.97 (m, 1H), 2.91-2.83 (m, 4H)-2.55 (s, 3H), 2.53-2.44 (m, 1H), 1.87 (qd, J = 8.2, 12.9 Hz, 1H) |
| 57 | 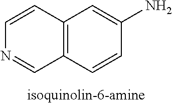 [SMILES: Nc1ccc2cnccc2c1] | 1 | 410.1 | (400 MHz, DMSO-d6) δ 13.23 (br s, 1H), 10.49 (s, 1H), 9.18 (s, 1H), 8.42 (d, J = 5.8 Hz, 1H), 8.22 (br d, J = 16.8 Hz, 2H), 8.07 (d, J = 8.9 Hz, 1H), 7.91 (s, 1H), 7.73 (d, J- = 5.8 Hz, 1H), 7.67-7.60 (m, 2H), |
| | | | | 4.20 (s, 2H), 2.90 (s, 3H), 2.45 |
| | | | | (s, 3H) |
| 58 | 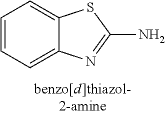 | 1 | 416.1 | (400 MHz, DMSO-d6) δ 13.17 (br s, 1H), 12.47 (br s, 1H), 8.20 (s, 1H), 7.98 (d, J = 7.8 Hz, 1H), 7.91 (s, 1H), 7.76 (d, J = 7.9 Hz, 1H), 7.64 (s, 1H), 7.44 (t, J- = 7.2 Hz, 1H), 7.34-7.28 (m, 1H), 4.32 (s, 2H), 2.91 (s, 3H), 2.46 (s, 3H) |
| 59 | 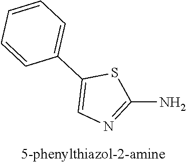 [SMILES: Nc1ncc(-c2ccccc2)s1] | 1 | 442.1 | (400 MHz, DMSO-d6) δ 13.18 (br s, 1H), 12.31 (br s, 1H)- 8.20 (s, 1H), 8.28-8.14 (m, 1H), 7.93-7.85 (m, 2H), 7.66- 7.55 (m, 3H), 7.42 (t, J- = 7.7 Hz, 2H), 7.34-7.28 (m, 1H), 4.27 (s, 2H), 3.30 (s, 1H), 2.90 (s, 3H), 2.46 (s, 3H) |
| 60 | 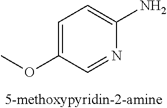 | 1 | 390.1 | (400 MHz, DMSO-d6) δ 10.45 (br s, 1H), 8.21 (s, 1H), 8.05 (d, J- = 3.0 Hz, 1H), 7.94-7.86 (m, 2H), 7.65 (s, 1H), 7.44 (dd, J = 3.1, 9.1 Hz, 1H), 4.17 (s, 2H), 3.82 (s, 3H), 2.85 (s, 3H) 150- ydroxyams, 3H) |
| 61 | 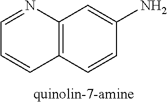 [SMILES: Nc1ccc2cccnc2c1] | 1 | 410.1 | (400 MHz, DMSO-d6) δ 10.38 (s, 1H), 8.80 (dd, J = 1.-, 4.2 Hz, 1H), 8.32-8.22 (m, 2H), 7.91-7.85 (m, 2H), 7.71-7.56 (m, 2H), 7.42-7.34 (m, 1H), 4.15 (s, 2H), 2.85 (s, 3H), 2.41 (s, 3H) |
| 62 | 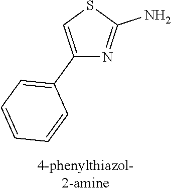 [SMILES: Nc1nc(-c2ccccc2)cs1] | 1 | 442.1 | (400 MHz, DMSO-d6)-8.16 (s, 1H), 7.91-7.83 (m, 3H), 7.61 (d, J = 1.5 Hz, 1H), 7.50 (s, 1H), 7.38 (t, J- = 7.7 Hz, 2H), 7.32-7.22 (m, 1H), 4.18 (s, 2H), 2.80 (s, 3H), 2.42 (s, 3H) |
| 63 | 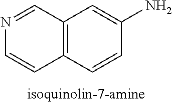 [SMILES: Nc1ccc2ccncc2c1] | 1 | 410.1 | (400 MHz, DMSO-d6) δ = 10.41 (s, 1H), 9.18 (s, 1H), 8.38 (d, J = 5.5 Hz, 1H), 8.34 (s, 1H)-8.16 (s, 1H), 7.92-7.85 (m, 2H), 7.75-7.69 (m, 2H), 7.62 (d, J = 0.9 Hz, 1H), 4.15 (s, 2H), 2.86 (s, 3H) 150- ydroxyams, 3H) |
| 64 | 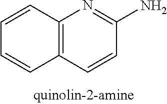 [SMILES: Nc1ccc2ccccc2n1] | 1 | 410.1 | (400 MHz, DMSO-d6) δ 10.84 (br s, 1H), 8.31 (d, J = 9.0 Hz, 1H), 8.16 (s, 1H), 8.07 (br d, J = 7.5 Hz, 1H), 7.92-7.84 (m, 2H), 7.80-7.73 (m, 1H), 7.68 (t, J = 7.8 Hz, 1H), 7.62 (s, 1H), 7.46 (t, J = 7.3 Hz, 1H), 4.23 (br s, 2H), 2.85 (s, 3H), 2.42 (s, 3H), 2.03 (d, J = 0.9 Hz, 1H) |
| 65 | 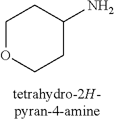 | 2 | 367.1 | (400 MHz, METHANOL-d4) δ = 8.15 (s, 1H), 7.92 (s, 1H)- 7.75 (s, 1H), 3.97-3.88 (m, 5H), 3.53-3.43 (m, 2H), 2.82 (s, 3H), 2.56 (s, 3H), 1.81 (br d, - = 10.3 Hz, 2H), 1.57-1.45 (m, 2H) |
| 66 | 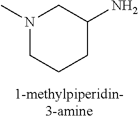 | 2 | 380.2 | (400 MHz, METHANOL-d4) δ 8.15 (s, 1H), 7.92 (s, 1H)- 7.74 (s, 1H), 3.96-3.87 (m, 3H), 2.83 (s, 3H), 2.62 (br s, 2H), 2.56 (s, 3H)-2.29 (s, 3H), 2.17-1.94 (m, 2H), 1.85-1.71 (m, 2H), 1.68-1.54 (m, 1H), 1.32 (br d, J = 8.7 Hz, 1H) |
| 67 | 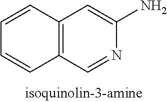 [SMILES: Nc1cc2ccccc2cn1] | 1 | 410.1 | (400 MHz, DMSO-d6) δ 13.18 (s, 1H), 10.63 (s, 1H), 9.11 (s, 1H), 8.30 (s, 1H), 8.16 (d, J = 1.3 Hz, 1H), 8.02 (d, J = 7.9 Hz, 1H), 7.86 (d, J-10.5 Hz, 2H), 7.73-7.67 (m, 1H), 7.62 (d, J- = 0.9 Hz, 1H), 7.54-7.46 (m, 1H), 4.21 (s, 2H), 2.86 (s, 3H), 2.41 (s, 3H) |
| 68 | 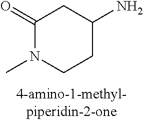 | 2 | 394.2 | (400 MHz, METHANOL-d4) δ 8.10 (s, 1H), 7.87 (dd, J = 0.9, 1.5 Hz, 1H), 7.70 (d, J- = 1.1 Hz, 1H), 4.15-4.05 (m, 1H), 3.90 (d, J = 3.9 Hz, 2H), 3.36 (dd, J = 5.0, 7.2 Hz, 2H), 2.91 (s, 3H), 2.79 (s, 3H), 2.61 (ddd, J = 1.1, 5.6, 17.4 Hz, 1H), 2.26 |
| | | | | (dd, J = 8.7-17.4 Hz, 1H), |
| | | | | 2.06-1.96 (m, 1H), 1.81 (tdd, |
| | | | | J = 7.4, 9.8, 13.2 Hz, 1H) |
| 69 | 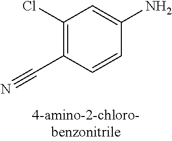 | 1 | 418.1 | (400 MHz, DMSO-d6) δ-8.15 (s, 1H), 7.93-7.83 (m, 3H), 7.60-7.52 (m, 2H), 4.13 (s, 2H), 2.81 (s, 3H), 2.41 (s, 3H) |
| 72 | 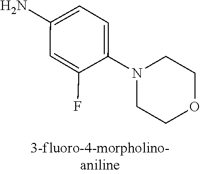 [SMILES: Nc1ccc(N2CCOCC2)c(F)c1] | 1 | 462.2 | (400 MHz, METHANOL-d4) δ 8.11 (s, 1H), 7.88 (s, 1H), 7.72 (s, 1H), 7.40 (dd, J = 2.4, 14.7 Hz, 1H), 7.14 (br d, J- = 8.6 Hz, 1H), 7.02-6.92 (m, 1H)- 4.07 (s, 2H), 3.82-3.79 (m, 4H), 3.02-2.99 (m, 4H), 2.86 (s, 3H), 2.49 (s, 3H) |
| 73 | 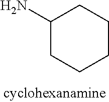 | 2 | 365.2 | (400 MHz, METHANOL-d4) δ 8.10 (s, 1H), 7.88 (s, 1H), 7.70 (s, 1H)-3.86 (s, 2H), 3.66- 3.58 (m, 1H), 2.77 (s, 3H)-2.51 (s, 3H), 1.83-1.62 (m, 5H), 1.37-1.17 (m, 5H) |
| 75 | 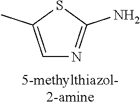 | 1 | 380.1 | (400 MHz, DMSO-d6) δ 8.15 (s, 1H), 7.86 (s, 1H), 7.58 (s, 1H), 7.08 (d, J = 1.1 Hz, 1H), 4.16 (s, 2H), 2.81 (s, 3H), 2.41 (s, 3H), 2.29 (d, J = 1.1 Hz, 3H) |
| 76 | 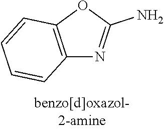 | 1 | 400.1 | (400 MHz, DMSO-d6)-8.15 (s, 1H), 7.96-7.78 (m, 1H), 7.63 (d, J- = 0.9 Hz, 1H), 7.54-7.38 (m, 2H), 7.24-7.05 (m, 2H), 4.25 (br s, 2H), 2.75 (s, 3H), 2.45 (br s, 3H) |
| 77 | 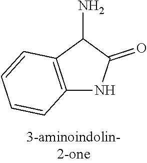 [SMILES: NC1C(=O)Nc2ccccc21] | 3 | 414.0 | (400 M-z, DMSO-d6) δ 13.40- 13.14 (m, 1H), 10.42 (s, 1H), 8.92 (d, J = 8.2 Hz, 1H), 8.17 (s, 1H), 7.90 (s, 1H)-7.62 (s, 1H), 7.24-7.14 (m, 1H), 7.07 (d, J- = 7.5 Hz, 1H), 6.97-6.86 (m, 1H), 6.79 (d, J = 7.7 Hz, 1H), 5.13 (d, J = 8.2 Hz, 1H), 3.88 (q, J = 16.1 Hz, 2H), 2.72 (s, 3H), 2.46 (s, 3H), 2.06 (s, 1H) |
| 78 | 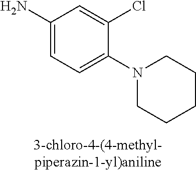 [SMILES: Nc1ccc(N2CCCCC2)c(Cl)c1] | 1 | 491.2 | (400 MHz, METHANOL-d4) δ 8.12 (s, 1H), 7.89 (s, 1H), 7.73 (d, J = 1.1 Hz, 1H), 7.65 (d, J = 2.6 Hz, 1H), 7.34 (dd, J = 2.5, 8.7 Hz, 1H), 7.09 (d, J = 8.8 Hz, 1H)-4.09 (s, 2H), 3.09-2.98 (m, 4H)-2.89 (s, 3H), 2.71-2.55 (m, 4H), 2.50 (s, 3H), 2.36 (s, 3H) |
| 79 | 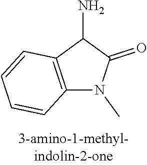 | 3 | 428.1 | (400 MHz, METHANOL-d4) δ 8.12 (s, 1H), 7.89 (s, 1H), 7.71 (d, J = 0.9 Hz, 1H), 7.34 (t, J = 7.7 Hz, 1H), 7.25 (d, J- = 7.5 Hz, 1H), 7.12-7.04 (m, 1H), 6.99 (d, J- = 8.2 Hz, 1H), 5.30-5.25 (m, 1H), 3.94 (s, 2H), 3.23 (s, 3H), 2.84 (s, 3H), 2.52 (s, 3H) |
| 83 | 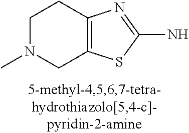 | 4 | 414.0 | (400 MHz, METHANOL-d4) δ 8.14 (s, 1H), 7.91 (s, 1H), 7.75 (d, J = 1.0 Hz, 1H), 4.64 (br d, J = 14.9 Hz, 1H), 4.36 (br d, J = 14.8 Hz, 1H)-4.28 (s, 2H), 3.88-3.78 (m, 1H), 3.62- 3.48 (m, 1H), 3.16-3.07 (m, 5H), 2.95 (s, 3H), 2.53 (s, 3H) |
| 89 | I-15 | 1 | 500.4 | (DMSO-d6, 400 MHz): δ 9.84 |
| | | | | (s, 1H), 8.19 (s, 1H), 7.90 (s, |
| | | | | 1H), 7.63 (s, 1H), 6.99 (d, J = |
| | | | | 2.0 Hz, 1H), 6.94-6.88 (m, 1H), |
| | | | | 6.86-6.77 (m, 1H), 4.20-4.11 |
| | | | | (m, 2H), 4.02 (s, 2H), 3.94- |
| | | | | 3.90 (m, 2H), 3.87-3.84 (m, |
| | | | | 1H), 3.43 (t, J = 10.8 Hz, 2H), |
| | | | | 3.25-3.15 (m, 2H), 2.78 (s, |
| | | | | 3H), 2.46 (s, 3H), 1.75-1.62 |
| | | | | (m, 2H), 1.61-1.51 (m, 2H) |
| 90 | 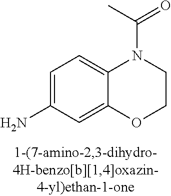 | 1 | 458.1 | (400 MHz, methanol-d₄) δ 9.-6 (br. s, 1H), 8.16-8.09 (m, 1H), 7.94-7.85 (m, 1H), 7.77- 7.68 (m, 1H), 7.29-7.20 (m, 1H), 7.08-6.94 (m, 1H), 4.29 (t, J = 4.0 Hz, 2H), 4.10 (s, 2H), 3.91 (t, J = 4.6 Hz, 2H), 2.88 (s, 3H), 2.52 (s, 3H), 2.30 (s, 3H) |
| 94 | I-18 | 1 | 472.1 | (DMSO-d₆, 400 MHz) δ 13.30 |
| | | | | (br. s, 1H), 9.81 (br. s, 1H), 8.19 |
| | | | | (s, 1H), 7.90 (s, 1H), 7.63 (d, |
| | | | | J = 0.8 Hz, 1H), 7.00 (d, J = 2.4 |
| | | | | Hz, 1H), 6.87 (dd, J = 8.4, 2.0 |
| | | | | Hz, 1H), 6.23 (d, J- = 8.8 Hz, |
| | | | | 1H), 4.77-4.73 (m, 2H), 4.64 |
| | | | | (t, J- = 6.4 Hz, 2H), 4.53-4.45 |
| | | | | (m, 1H), 4.29-4.25 (m, 2H)- |
| | | | | 4.01 (s, 2H), 3.17-3.12 (m, |
| | | | | 2H), 2.79 (s, 3H), 2.46 (s, 3H) |
| 102 | 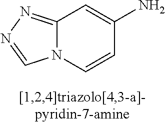 | 3 | 400.1 | (400 MHz, DMSO-d6) δ 9.11 (s, 1H), 8.46 (d, J = 7.3 Hz, 1H), 8.18 (s, 1H), 7.96 (s, 1H), 7.89 (s, 1H), 7.62 (s, 1H), 6.93 (dd, J = 1.8, 7.5 Hz, 1H), 4.16 (s, 2H), 2.86 (s, 3H), 2.44 (s, 3H) |
| 104 | 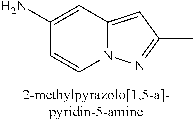 | 1 | 413.0 | (CD₃OD, 400 MHz) δ 8.29 (d, J = 7.6 Hz, 1H)-8.13 (s, 1H), 7.91-7.86 (m, 2H), 7.74 (s, 1H), 6.81 (dd, J = 2.4, 7.6 Hz, 1H), 6.25 (s, 1H), 4.15 (s, 2H), 2.91 (s, 3H), 2.51 (s, 3H), 2.40 (s, 3H) |
| 105 | I-25 | 1 | 488.1 | (DMSO-d₆, 400 MHz): δ 13.31 |
| | | | | (br.s, 1H), 9.80 (br.s, 1H), 8.19 |
| | | | | (d, J = 1.2 Hz, 1H), 7.90 (s, 1H), |
| | | | | 7.63 (d, J = 1.2 Hz, 1H), 6.98 (d, |
| | | | | J = 2.4 Hz, 1H), 6.92 (d, J = 9.2 |
| | | | | Hz, 1H), 6.82 (dd, J = 2.4 Hz, |
| | | | | 8.8 Hz, 1H), 4.75 (t, J = 5.2 Hz, |
| | | | | 1H), 4.14 (t, J = 4.0 Hz, 2H), |
| | | | | 4.02(s, 2H), 3.46 (d, J = 5.6 Hz, |
| | | | | 2H), 3.25 (t, J = 4.4 Hz, 2H), |
| | | | | 2.79 (s, 3H), 2.46 (s, 3H), 1.21 |
| | | | | (s, 6H) |
| 114 | 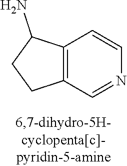 | 3 | 400.2 | (400 MHz, METHANOL-d4) δ 8.46 (s, 1H), 8.37 (d, J = 5.1 Hz, 1H), 8.15 (s, 1H), 7.93 (s, 1H), 7.77 (s, 1H), 7.34 (d, J = 5.1 Hz, 1H), 5.46 (t, J = 8.3 Hz, 1H), 4.02 (d, J- = 4.3 Hz, 2H), 3.14-3.06 (m, 1H), 3.01-2.93 (m, 1H)-2.88 (s, 3H), 2.59- 2.54 (m, 4H), 2.00-1.92 (m, 1H) |
| 115 | 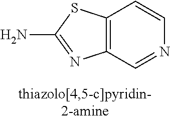 | 4 | 417.1 | (400 MHz, DMSO-d6) δ 2.46 (s, 3 H) 2.91 (s, 3 H) 4.36 (s, 2H) 7.65 (s, 1 H) 7.91 (s, 1 H) 8.07 (d, J = 5.14 Hz, 1 H) 8.19 (s, 1 H) 8.41 (d, J = 5.38 Hz, 1H) 9.02 (s, 1 H) |
| 116 | 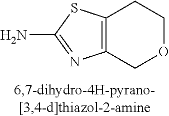 | 4 | 422.0 | (400 MHz,-METHANOL-d4) δ 2.51-2.57 (m, 3 H) 2.76-2.88 (m, 2 H) 2.91-2.97 (m, 3 H) 3.19-3.25 (m, 1 H) 3.49-3.56 (m, 1 H) 3.98 (t, J- = 5.44 Hz, 1 H) 4.18-4.23 (m, 1 H-4.28 (s, 1 H) 4.62-4.65 (m, 1 H) 7.70- 7.77 (m, 1 H) 7.89-7.93 (m, 1 |
| | | | | H) 8.14-8.18 (m, 1 H) |
| 127 | 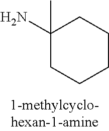 | 2 | 379.2 | (400 MHz, DMSO-d6) δ 13.44 (br s, 1H), 8.20 (s, 1H), 7.91 (s, 1H), 7.64 (s, 1H), 7.32 (s, 1H), 3.85 (s, 2H), 2.72 (s, 3H), 2.49 (s, 3H), 1.97 (br d, J-12.9 Hz, 2H), 1.47-1.36 (m, 5H), 1.28- 1.18 (m, 6H) |
| 130 | I-36 | 3 | 467.1 | (400 MHz, DMSO-d6) δ 13.19 |
| | | | | (s, 1H), 10.40 (s, 1H), 8.39 (d, |
| | | | | J = 7.5 Hz, 1H), 8.17 (d, J = 1.1 |
| | | | | Hz, 1H), 8.08 (s, 1H), 7.89 (s, |
| | | | | 1H), 7.62 (s, 1H), 7.50 (s, 1H), |
| | | | | 7.00 (dd, J = 2.0, 7.7 Hz, 1H), |
| | | | | 4.13 (s, 2H), 2.85 (s, 3H), 2.44 |
| | | | | (s, 3H) |

### Synthesis of 2-((N,5-dimethyl-1H-indazole)-7-sulfonamido)-N-(1-ethyl-2-oxo-1,2-dihydropyridin-4-yl)acetamide (Example 14)

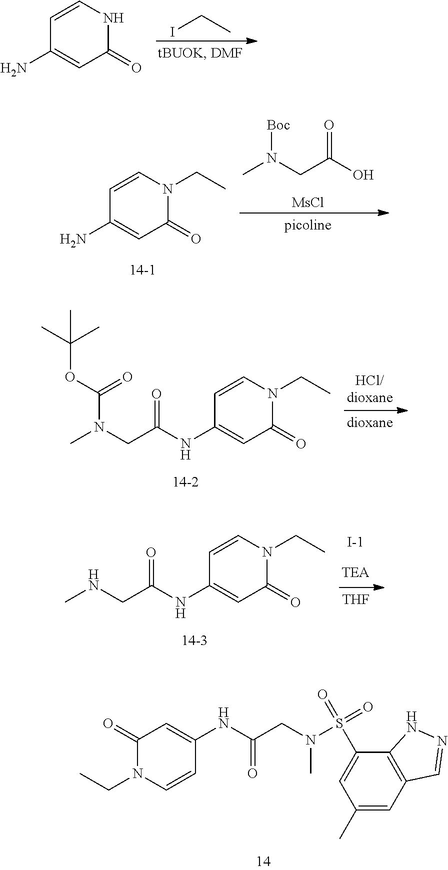

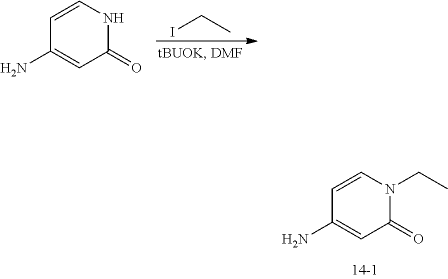

To a solution of 4-aminopyridin-2(1H)-one (0.500 g, 4.54 mmol) in dimethyl formamide (5 mL) was added potassium tert-butoxide (1.02 g, 9.08 mmol). The mixture was stirred at 0° C. for 0.5 hour then iodoethane (0.779 g, 4.99 mmol) was added to the mixture and stirred at 0° C. for 2 hours. The mixture was poured into water (10 mL) and dried by lyophilization. The residue was purified by column chromatography (SiO₂, petroleum ether/ethyl acetate=1/1˜0/1, ethyl acetate/methanol=10/1) to afford 4-amino-1-ethylpyridin-2(1H)-one (14-1) (0.400 g, 43.1% yield) as a yellow solid.

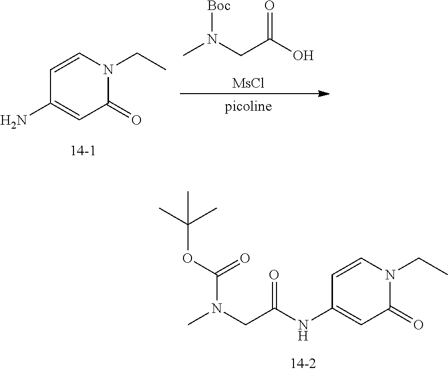

To a solution of 2-((tert-butoxycarbonyl)(methyl)amino)acetic acid (0.411 g, 2.17 mmol) and 14-1 (0.300 g, 2.17 mmol) in 3-picoline (3 mL) was added methanesulfonyl chloride (0.336 mL, 4.34 mmol) at 0° C. The mixture was stirred at 0° C. for 1 hour then the mixture was poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was triturated with petroleum ether/ethyl acetate=1/1 (10 mL) to afford tert-butyl(2-((1-ethyl-2-oxo-1,2-dihydropyridin-4-yl)amino)-2-oxoethyl)(methyl)carbamate (14-2) (0.350 g, 52.1% yield) as a yellow solid.

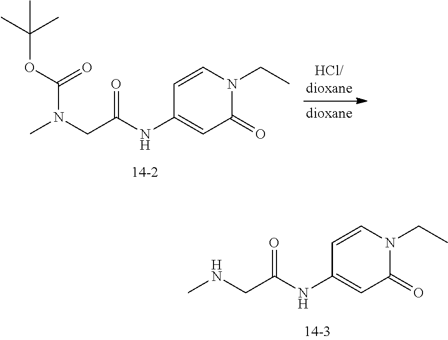

To a solution of 14-2 (0.350 g, 1.13 mmol) in dioxane (4 mL) was added a solution of HCl in dioxane (4 M, 4 mL, 16.0 mmol) at 0° C. The mixture was stirred at 25° C. for 1 hour then concentrated under reduced pressure to afford N-(1-ethyl-2-oxo-1, 2-dihydropyridin-4-yl)-2-(methylamino)acetamide (14-3) as the hydrochloride salt (0.278 g, 100% yield) as a white solid. No further purification was performed.

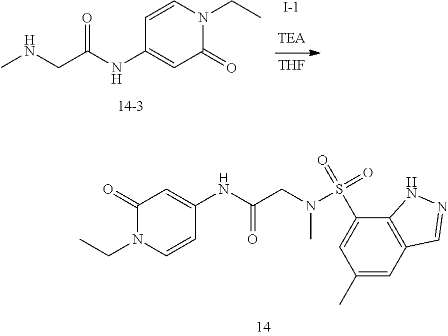 [SMILES: CCn1ccc(NC(=O)CNC)cc1=O ; [I-] ; "THF" ; CCn1ccc(NC(=O)CN(C)S(=O)(=O)c2cc(C)cc3cn[nH]c23)cc1=O]

To a solution of 14-3 (0.073 g, 0.347 mmol) and I-1 (0.080 g, 0.347 mmol) in tetrahydrofuran (1 mL) was added triethylamine (0.145 mL, 1.04 mmol). The mixture was stirred at 25° C. for 3 hours. The mixture was poured into water (100 mL), causing a yellow solid to precipitate out. The formed solid was collected by filtration to afford 2-(N,5-dimethyl-1H-indazole-7-sulfonamido)-N-(1-ethyl-2-oxo-1,2-dihydropyridin-4-yl)acetamide (14) (0.064 g, 44.1% yield) as a yellow solid.

LCMS of 14: m/z 404.1 [M+H]⁺

¹H-NMR of 14: (DMSO-d6, 400 MHz): δ 13.18 (br. s, 1H), 10.15 (s, 1H), 8.18 (s, 1H)-7.90 (s, 1H), 7.62-7.58 (m, 2H), 6.61 (d, J=2.4 Hz, 1H), 6.31 (dd, J=7.2, 2.0 Hz, 1H), 4.11 (s, 2H), 3.81 (q, J=7.2 Hz, 2H), 2.83 (s, 3H), 2.46 (s, 3H), 1.16 (t, J=6.8 Hz, 3H) The following compound was synthesized in a similar manner as 14 using the reactant indicated.

| | | LCMS | |
|---|---|---|---|
| Example | Reactant | m/z | ¹H-NMR |
| 16 | 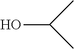 | 418.0 | (CDCl₃, 400 MHz): δ 11.84 (br. s, 1H), 9.10 (br. s, 1H), 8.13 (s, 1H), 7.84 (s, 1H), 7.68 (s, 1H), 7.31 (d, J = 7.6 Hz, 1H), 6.86 (d, J = 6.8 Hz, 1H), 6.66 (d, J- = 2.4 Hz, 1H), 5.38-5.16 (m, 1H), 4.01 (s, 2H), 2.87 (s, 3H), 2.54 (s, 3H), 1.35 (d, J = 6.8 Hz, 6H) |

### Synthesis of N-(3-chloro-4-methoxyphenyl)-2-((N-ethyl-5-methyl-1H-indazole)-7-sulfonamido)acetamide (Example 15)

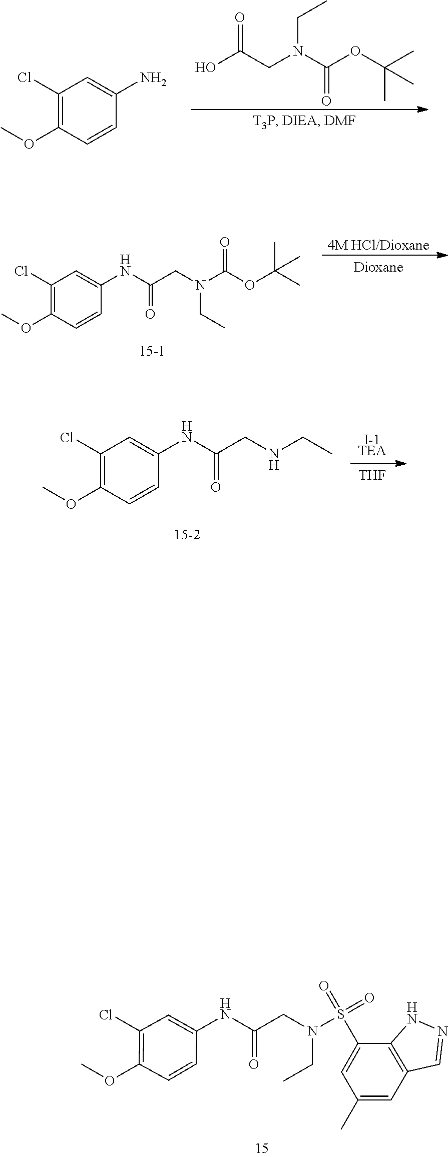

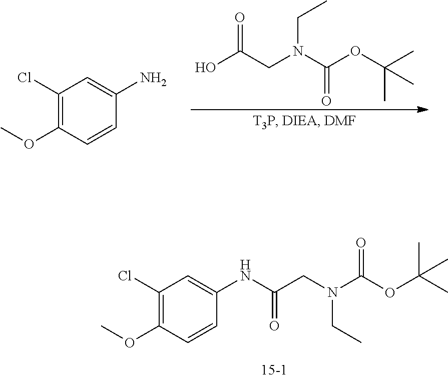

To a solution of 3-chloro-4-methoxyaniline (0.800 g, 5.08 mmol) and 2-((tert-butoxycarbonyl)(ethyl)amino)acetic acid (1.03 g, 5.08 mmol) in N,N-dimethylformamide (10 mL) was added T₃P (4.20 g, 6.60 mmol, 50% purity in ethyl acetate) and diisopropylethylamine (1.64 g, 12.7 mmol) at 0° C. The mixture was stirred at 0° C. for 1.5 hours. The mixture was poured into ice water (50 mL) and extracted with ethyl acetate (100 mL*3). The combined organic layers were washed with a saturated aqueous solution of sodium bicarbonate (100 mL), followed by 1N HCl (100 mL) and brine (100 mL*2), then dried over anhydrous sodium sulfate, filtered, and the filtrate was concentrated under reduced pressure to give tert-butyl (2-((3-chloro-4-methoxyphenyl)amino)-2-oxoethyl)(ethyl)carbamate (15-1) (1.50 g, 4.38 mmol, 86.2% yield) as black brown solid.

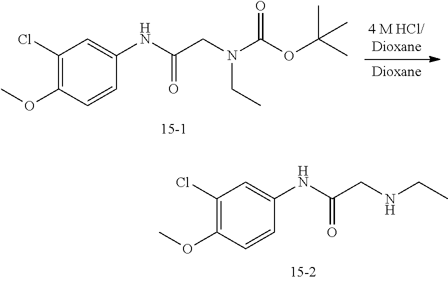

To a solution of 15-1 (1.50 g, 4.38 mmol) in 1,4-dioxane (15 mL) at 0° C. was added a solution of HCl in 1,4-dioxane (4 M, 7.20 mL) by drop-wise. The mixture was stirred at 25° C. for 2 hours and then at 40° C. for another 2 hours. The mixture was concentrated under reduced pressure to afford N-(3-chloro-4-methoxyphenyl)-2-(ethylamino)acetamide (15-2) as the hydrochloric acid salt (1.00 g, 79.7% yield) as a gray solid. No further purification was performed.

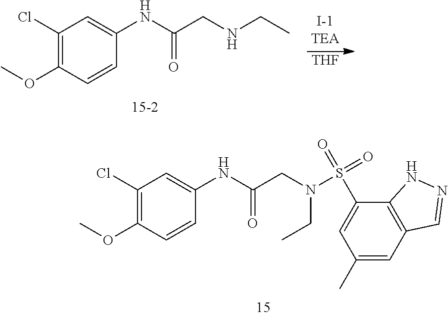

To a solution of 15-2 (0.105 g, 0.377 mmol) and triethylamine (0.181 mL, 1.30 mmol) in tetrahydrofuran (1 mL) was added I-1 (0.100 g, 0.434 mmol) at 20° C. The mixture was stirred at 20° C. for 1.5 hours then poured into water (20 mL) and extracted with ethyl acetate (10 mL*3). The combined organic layers were washed with brine (20 mL), dried over anhydrous sodium sulfate, filtered, and the filtrate was concentrated under reduced pressure. The residue was triturated with ethyl acetate (10 mL) to afford N-(3-chloro-4-methoxyphenyl)-2-(N-ethyl-5-methyl-1H-indazole-7-sulfonamido) acetamide (15) (0.041 g, 21.3% yield) as a white solid.

LCMS of 15: m/z 437.3 [M+H]⁺

¹H-NMR of 15: (DMSO-d6, 400 MHz): δ 13.34 (br. s, 1H), 10.13 (br. s, 1H), 8.18 (s, 1H)-7.88 (s, 1H), 7.72-7.62 (m, 2H), 7.41-7.30 (m, 1H), 7.11 (d, J=8.0 Hz, 1H), 4.19 (s, 2H)-3.82 (s, 3H), 3.30-3.24 (m, 2H), 2.44 (s, 3H), 0.97 (t, J=6.8 Hz, 3H)

The following compounds were synthesized in a similar manner as 15 using the reactant indicated.

| | | LCMS | |
|---|---|---|---|
| Example | Reactant | m/z | ¹H-NMR |
| 18 | I-10 | 492.1 | (CDCl₃, 400 MHz): δ 11.37 (br. s, 1H), |
| | | | 8.21 (s, 1H), 8.13 (s, 1H), 7.84 (s, 1H), |
| | | | 7.164 (t, J = 2.4 Hz, 2H), 7.41 (d, J = |
| | | | 8.4 Hz, 1H), 7.02 (d, J = 8.4 Hz, 1H)- |
| | | | 3.92 (s, 2H), 3.90-3.88 (m, 4H), 3.77 |
| | | | (q, J- = 7.2 Hz, 2H), 3.05-3.03 (m, |
| | | | 4H), 2.55 (s, 3H), 1.16 (t, J =7.6 Hz, |
| | | | 3H) |
| 88 | 30-2 | 444.1 | (CDCl₃, 400 MHz): δ 11.44 (br. s, 1H), |
| | | | 8.12 (s, 1H), 7.93 (br. s, 1H), 7.82 (s, |
| | | | 1H), 7.66 (d, J- = 0.8 Hz, 1H), 7.01- |
| | | | 6.97 (m, 2H), 6.66-6.60 (m, 1H), 4.34- |
| | | | 4.28 (m, 2H)-3.92 (s, 2H), 3.36-3.31 |
| | | | (m, 2H), 3.27-3.23 (m, 2H), 2.88 (s, |
| | | | 3H), 2.54 (s, 3H), 1.15 (t, J = 7.2 Hz, |
| | | | 3H) |
| 160 | 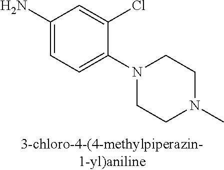 | 505.1 | (400 MHz, CDCl₃) δ 12.00 (br. s, 1H), 8.91 (br. s, 1H), 8.12 (s, 1H), 7.84 (m, 2H), 7.71 (s, 1H), 7.42 (dd, J = 2.4, 8.8 Hz, 1H), 6.98 (d, J = 8.4 Hz, 1H)-4.09 (s, 2H), 3.33-3.28 (m, 2H), 3.18 (s, 4H-, 2.95(s, 4H), 2.67-2.61 (m, 3H), 2.54 (s, 3H), 1.13 (t, J = 7.2 Hz, 3H) |

### Synthesis of 2-((N,5-dimethyl-1H-indazole)-7-sulfonamido)-N-(1,6-dimethyl-2-oxo-1,2-dihydropyridin-4-yl)acetamide (Example 17)

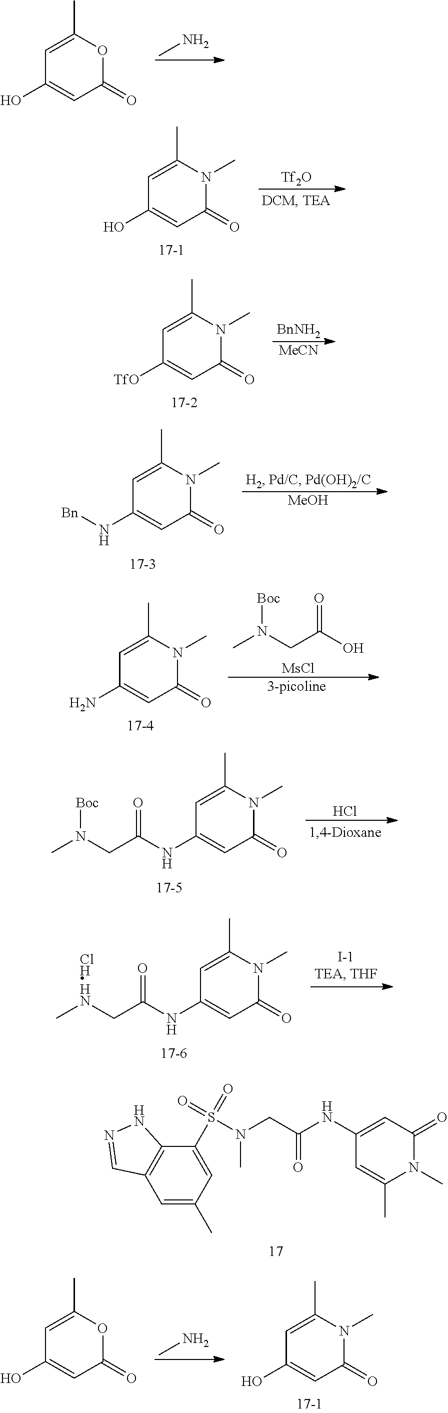

To a mixture of 4-hydroxy-6-methyl-2H-pyran-2-one (3.00 g, 23.8 mmol) in water (10 mL) was added methylamine (24.6 g, 238 mmol) at 20° C. The mixture was stirred at 100° C. for 2 hours, and then concentrated under reduced pressure. The residue was purified by reverse phase flash chromatography (0.1% trifluoroacetic acid in water, acetonitrile) to afford 4-hydroxy-1,6-dimethylpyridin-2(1H)-one (17-1) (3.00 g, 89.8% yield) as a white solid.

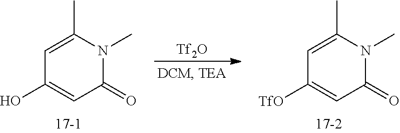

To a solution of 17-1 (3.00 g, 21.6 mmol) and triethylamine (9.00 mL, 64.7 mmol) in dichloromethane (30 mL) was added Tf₂O (8.55 g, 30.3 mmol) drop-wise at 0° C. under nitrogen. The mixture was stirred for 2 hours at 20° C. under nitrogen atmosphere, then concentrated under reduced pressure. The residue was purified by reverse phase flash chromatograpy (0.1% trifluoroacetic acid in water, acetonitrile) to afford 1,6-dimethyl-2-oxo-1,2-dihydropyridin-4-yl trifluoromethanesulfonate (17-2) (1.50 g, 22.3% yield) as a red gum.

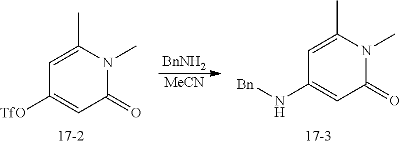

To a solution of 17-2 (1.50 g, 5.53 mmol) in acetonitrile (15 mL) was added N-ethyl-N-isopropylpropan-2-amine (2.23 g, 17.2 mmol) and benzylamine (1.78 g, 16.6 mmol). The mixture was stirred for 12 hours at 85° C. The mixture was diluted with ethyl acetate (80 mL), washed with brine (80 mL*3), dried over anhydrous sodium sulfate, filtered, and the filtrate was concentrated under reduced pressure. The residue was purified by column chromatography (SiO₂, ethyl acetate to ethyl acetate:methanol=5:1) to afford 4-(benzylamino)-1,6-dimethylpyridin-2(1H)-one (17-3) (0.400 g, 1.42 mmol, 25.7% yield) as a yellow solid.

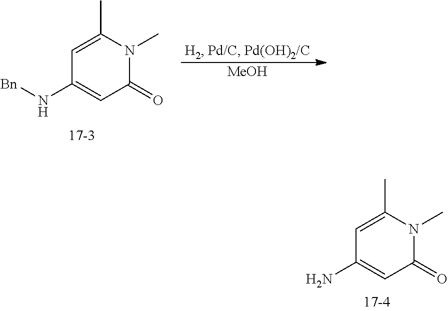

To a solution of 17-3 (0.400 g, 1.75 mmol) in methanol (10 mL) was added Pd/C (0.030 g, 5% purity on charcoal) and Pd(OH)₂/C (0.030 g, 10% purity on charcoal) under nitrogen. The mixture was degassed with hydrogen three times and stirred for 36 hour at 20° C. under a hydrogen atmosphere (15 PSI). The mixture was filtered and the filtrate was concentrated under reduced pressure to afford 4-amino-1,6-dimethylpyridin-2(1H)-one (17-4) (0.180 g, 74.4% yield) as colorless gum. No further purification was performed.

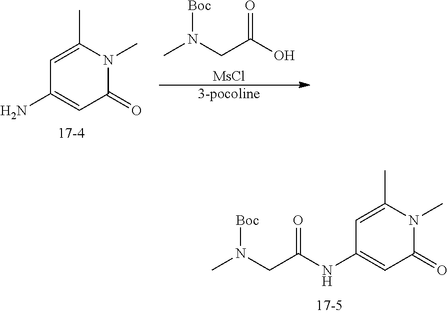

To a solution of 17-4 (0.180 g, 1.30 mmol) and 2-((tert-butoxycarbonyl)(methyl)amino)acetic acid (0.250 g, 1.32 mmol) in 3-picoline (3 mL) was added methanesulfonyl chloride (0.300 g, 2.62 mmol) at 0° C. drop-wise. The mixture was stirred for 2 hours at 20° C. The reaction was poured into ice-water (20 mL) and extracted with ethyl acetate (20 mL*3). The combined organic phase was washed with brine (20 mL*3), dried over anhydrous sodium sulfate, filtered, and the filtrate was concentrated under reduced pressure. The residue was purified by reverse phase flash chromatography (0.1% trifluoroacetic acid in water, acetonitrile) to afford tert-butyl (2-((1,6-dimethyl-2-oxo-1,2-dihydropyridin-4-yl)amino)-2-oxoethyl)(methyl)carbamate (17-5) (0.030 g, 6.77% yield) as a yellow gum.

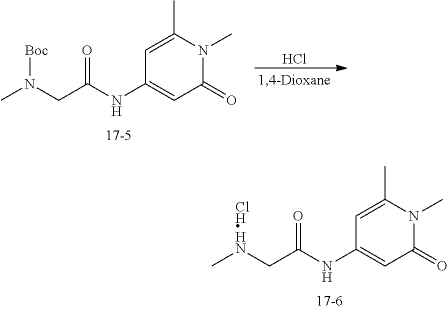

To a solution 17-5 (0.030 g, 0.088 mmol) in dioxane (1 mL) was added a solution of HCl in dioxane (4 M, 5 mL, 20 mmol)). The mixture was stirred 1 hour at 20° C., then concentrated under reduced pressure to afford N-(1,6-dimethyl-2-oxo-1,2-dihydropyridin-4-yl)-2-(methylamino)acetamide hydrochloride (17-6) (0.020 g, crude) as a yellow solid. No further purification was performed and the material was used directly.

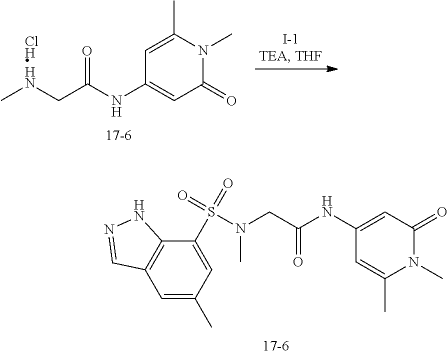

To a solution of 17-6 (0.020 g, crude) and triethylamine (0.025 g, 0.244 mmol) in tetrahydrofuran (1 mL) was added I-1 (0.020 g, 0.087 mmol). The mixture was stirred for 2 hours at 20° C. then the mixture was poured into water (50 mL) and extracted with ethyl acetate (50 mL*2). The combined organic phase was washed with brine (50 mL), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH₄HCO₃)-ACN]; B %: 18%-48%, 10 min) to afford 2-(N,5-dimethyl-1H-indazole-7-sulfonamido)-N-(1,6-dimethyl-2-oxo-1,2-dihydropyridin-4-yl)acetamide (17) (0.011 g, 31.9% yield) as a white solid.

LCMS of 17: m/z 404.1[M+H]⁺

¹H-NMR of 17: (400 MHz, CD₃OD) δ=8.12 (s, 1H), 7.89 (s, 1H), 7.72 (s, 1H), 6.74 (d, J=2.4 Hz, 1H), 6.49 (d, J=1.6 Hz, 1H), 4.12 (s, 2H), 3.50 (s, 3H), 2.90 (s, 3H), 2.52 (s, 3H), 2.40 (s, 3H)

### Synthesis of N-(3-chloro-4-morpholinophenyl)-2-((N,5,6-trimethyl-1H-indazole)-7-sulfonamido)acetamide (Example 19)

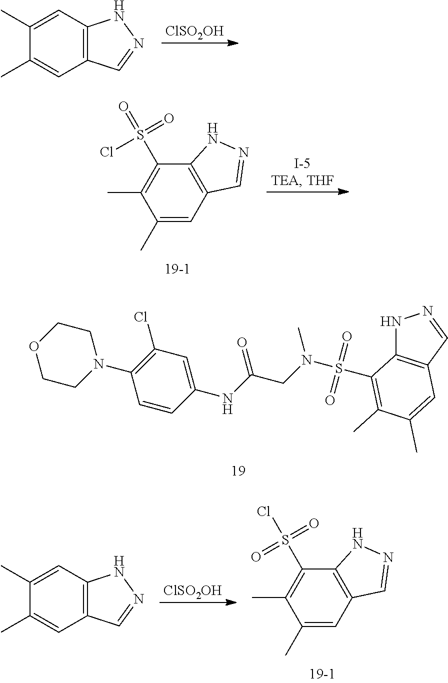

A round bottom flask was charged with chlorosulfonic acid (2 mL) and cooled to 0° C. To this was added 5, 6-dimethyl-1H-indazole (0.200 g, 1.37 mmol) slowly at 0° C. The mixture was stirred at 25° C. for 1 hour. The reaction mixture was poured into ice water (50 mL) causing a solid to precipitate out. The formed solid was collected by filtration and the cake was dissolved in ethyl acetate (50 mL), dried over anhydrous sodium sulfate, filtered, and the filtrate was concentrated under reduced pressure to afford 5, 6-dimethyl-1H-indazole-7-sulfonyl chloride (19-1) (0.280 g, 69.9% yield) as a yellow solid.

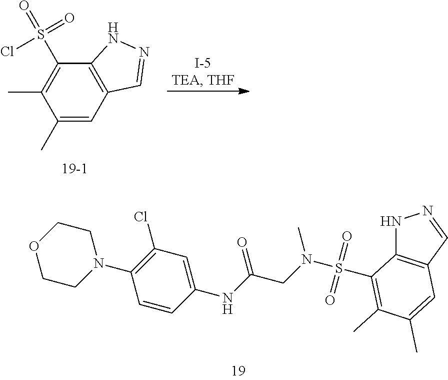 [SMILES: Cc1cc2cn[nH]c2c(S(=O)(=O)Cl)c1C ; "I-5TEA, THF" ; Cc1cc2cn[nH]c2c(S(=O)(=O)N(C)CC(=O)Nc2ccc(N3CCOCC3)c(Cl)c2)c1C]

To a mixture of 19-1 (0.060 g, 0.205 mmol) and I-5 (0.073 g, 0.205 mmol) in tetrahydrofuran (2 mL) was added triethylamine (0.086 mL, 0.614 mmol). The mixture was stirred at 25° C. for 2 hours, poured into water (50 mL), and extracted with ethyl acetate (20 mL*2). The combined organic layers were washed with brine (50 mL), dried over anhydrous sodium sulfate, filtered, and the filtrate was concentrated under reduced pressure. The residue was triturated with methanol (2 mL) and ac164ydroxile (2 mL) to afford N-(3-chloro-4-morpholinophenyl)-2-(N,5,6-trimethyl-1H-indazole-7-sulfonamido)acetamide (19) (0.049 g, 47.6% yield) as a white solid.

LCMS of 19: m/z: 492.1 [M+H]⁺

¹H-NMR of 19: (CDCl₃, 400 MHz): δ 11.26 (br. s, 1H), 8.25 (s, 1H), 8.07 (d, J=1.6 Hz, 1H), 7.85 (s, 1H), 7.65 (d, J=2.4 Hz, 1H), 7.40 (dd, J=2.4, 8.4 Hz, 1H), 7.03 (d, J=8.4 Hz, 1H)-3.94 (s, 2H), 3.91-3.85 (m, 4H), 3.06-3.01 (m, 4H), 2.95 (s, 3H), 2.67 (s, 3H), 2.46 (s, 3H)

The following compound was synthesized in a similar manner as 19 using the reactant indicated.

| | | LCMS | |
|---|---|---|---|
| Example | Reactant | m/z | ¹H-NMR |
| 20 | I-6 | 404.1 | (400 MHZ, DMSO-d₆): δ |
| | | | 12.65 (br. s, 1H), 10.12 |
| | | | (br. s, 1H), 8.10 (d, J = 1.2 |
| | | | Hz, 1H), 7.90 (s, 1H), |
| | | | 7.58 (d, J = 7.6 Hz, 1H), |
| | | | 6.64 (d, J = 2.0 Hz, 1H), |
| | | | 6.28 (dd, J = 2.0, 7.2 Hz, |
| | | | 1H), 4.14 (s, 2H), 3.32 (s, |
| | | | 3H), 2.83 (s, 3H), 2.57 |
| | | | (s, 3H), 2.38 (s, 3H) |

### Synthesis of N-(3-chloro-4-methoxyphenyl)-2-((N,5-dimethyl-1H-indole)-7-sulfonamido)acetamide (Example 21)

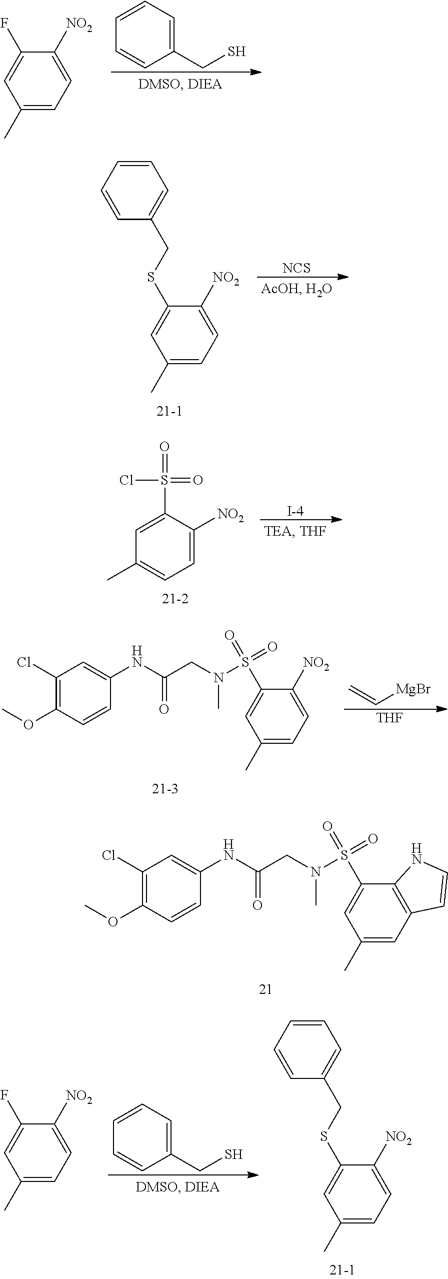

To a solution of 2-fluoro-4-methyl-1-nitrobenzene (1.00 g, 6.45 mmol) in dimethylsulfoxide (10 mL) was added diisopropylethylamine (2.25 mL, 12.9 mmol) and phenylmethanethiol (0.830 mL, 7.09 mmol). The mixture was stirred at 80° C. for 3 hours. The mixture was poured into water (100 mL) and extracted with ethyl acetate (100 mL*3). The combined organic layers were washed with brine (50 mL), dried over anhydrous sodium sulfate, filtered, and the filtrate was concentrated under reduced pressure. The residue was triturated with petroleum ether:ethyl acetate=10/1 (v/v, 5 mL) to afford benzyl(5-methyl-2-nitrophenyl)sulfane (21-1) (1.60 g, 95.0% yield) as a yellow solid.

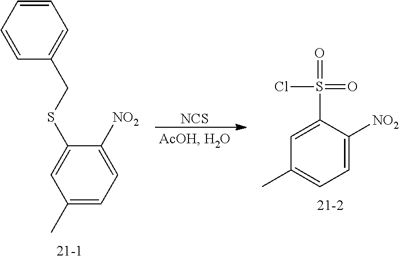 [SMILES: Cc1ccc([N+](=O)[O-])c(SCc2ccccc2)c1 ; "NCS" ; Cc1ccc([N+](=O)[O-])c(S(=O)(=O)Cl)c1]

To a solution of 21-1 (0.200 g, 0.771 mmol) in acetic acid (2 mL) and water (0.7 mL) was added NCS (0.410 g, 3.08 mmol) at 0° C. The mixture was stirred at 0-25° C. for 4 hours. The mixture was poured into water (10 mL) and extracted with ethyl acetate (10 mL*3). The combined organic layers were washed with brine (20 mL), dried over anhydrous sodium sulfate, filtered, and the filtrate was concentrated under reduced pressure to afford 5-methyl-2-nitrobenzene-1-sulfonyl chloride (21-2) (0.180 g, 85.0% yield) as a white solid.

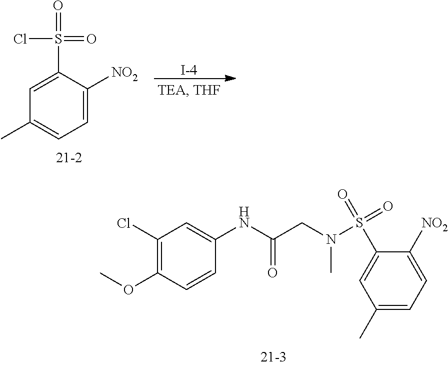

To a solution of 21-2 (0.180 g, 0.649 mmol) and I-4 (0.150 g, 0.566 mmol) in tetrahydrofuran (2 mL) was added triethylamine (0.270 mL, 1.95 mmol) at 0° C. The mixture was stirred at 0-25° C. for 2 hours. The mixture was poured into water (50 mL) and extracted with ethyl acetate (20 mL*3). The combined organic layers were washed with brine (20 mL), dried over anhydrous sodium sulfate, filtered, and the filtrate was concentrated under reduced pressure. The residue was triturated with petroleum ether: ethyl acetate=1/1 (v/v, 6 mL) to afford a N-(3-chloro-4-methoxyphenyl)-2-(N,5-dimethyl-2-nitrophenylsulfonamido)acetamide (21-3) (0.180 g, 57.0% yield) as a gray solid.

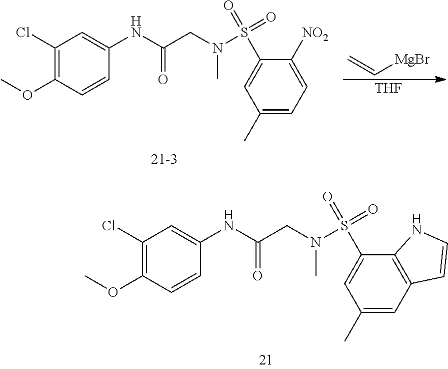

To a solution of 21-3 (0.080 g, 0.187 mmol) in tetrahydrofuran (3 mL) was added bromo(vinyl)magnesium (1.00 M, 1.87 mmoL, 1.87 mL) in tetrahydrofuran at −45° C. The mixture was stirred at −45° C. for 1 hour. The mixture was poured into water (50 mL) and extracted with ethyl acetate (30 mL*3). The combined organic layers were washed with brine (20 mL), dried over anhydrous sodium sulfate, filtered and the filtrate was concentrated under reduced pressure. The residue was triturated with ac167ydroxile (3 mL) to afford N-(3-chloro-4-methoxyphenyl)-2-(N,5-dimethyl-1H-indole-7-sulfonamido)acetamide (21) (0.026 g, 31.0% yield) as a white solid.

LCMS of Example 21: m/z 422.0 [M+H]⁺

¹H-NMR of Example 21: (CD₃OD, 400 MHz): δ 7.69 (s, 1H), 7.62 (d, J=2.4 Hz, 1H)-7.43 (s, 1H), 7.40-7.34 (m, 2H), 7.02 (d, J=8.8 Hz, 1H), 6.53 (d, J=3.2 Hz, 1H), 3.96 (s, 2H), 3.86 (s, 3H), 2.85 (s, 3H), 2.48 (s, 3H)

The following compounds were synthesized in a similar manner as 21 using the reactants indicated.

| | | LCMS | |
|---|---|---|---|
| Example | Reactant | m/z | ¹H-NMR |
| 22 | I-5 | 477.2 | (CDCl₃, 400 MHz): δ |
| | | | 9.49 (br. s, 1H), 8.25 (br. s, |
| | | | 1H), 7.73 (s, 1H), 7.65 |
| | | | (d, J −= 2.8 Hz, 1H), 7.42- |
| | | | 7.38 (m, 2H), 7.35-7.31 |
| | | | (m, 1H), 7.03 (d, J −= 8.8 |
| | | | Hz, 1H), 6.60-6.59 (m, 1H), |
| | | | 3.92-3.86 (m, 4H)- |
| | | | 3.79 (s, 2H), 3.07-3.00 |
| | | | (m, 4H), 2.89 (s, 3H), 2.52 |
| | | | (s, 3H) |
| 23 | I-6 | 389.1 | (CDCl₃, 400 MHz) δ 9.68 |
| | | | (br. s, 1H), 8.63 (br. s, |
| | | | 1H), 7.72 (s, 1H), 7.40 |
| | | | (s, 1H), 7.33 (t, J = 2.4 Hz, |
| | | | 1H), 7.26 (d, J = 7.6 Hz, |
| | | | 1H), 6.78 (d, J −= 2.0 Hz, |
| | | | 1H), 6.68-6.54 (m, 2H), |
| | | | 3.84 (s, 2H), 3.53 (s, 3H), |
| | | | 2.87 (s, 3H), 2.51 (s, 3H) |

### Synthesis of 2-((5-chloro-N-methyl-1H-indazole)-7-sulfonamido)-N-(1-methyl-2-oxo-1,2-dihydropyridin-4-yl)acetamide (Example 24)

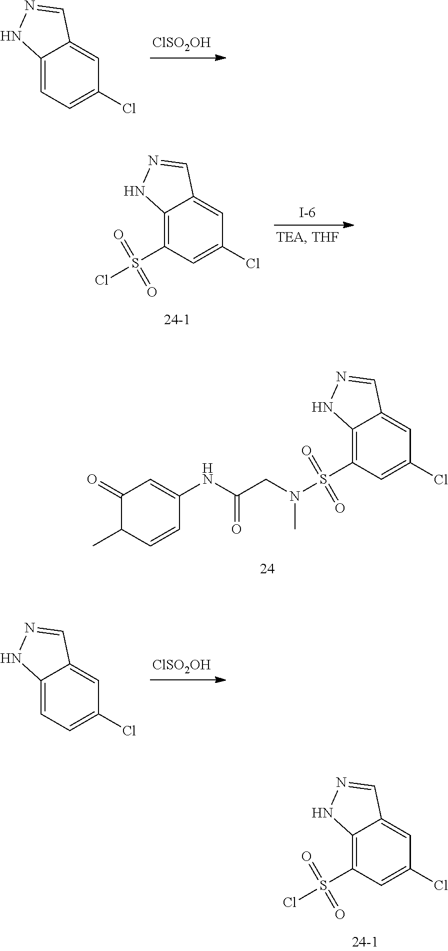

A mixture of 5-chloro-1H-indazole (0.200 g, 1.31 mmol) in sulfurochloridic acid (3.00 mL, 45.1 mmol) was stirred at 80° C. under nitrogen for 12 hours. After being cooled to 25° C., the mixture was poured into 50 mL of cooled water and extracted with ethyl acetate (40 mL*3). The combined organic layer was washed with brine (40 mL*2), dried over anhydrous sodium sulfate, filtered, and the filtrate was concentrated under reduced pressure to afford 5-chloro-1H-indazole-7-sulfonyl chloride (24-1) (0.300 g, 91.2% yield) as a yellow solid.

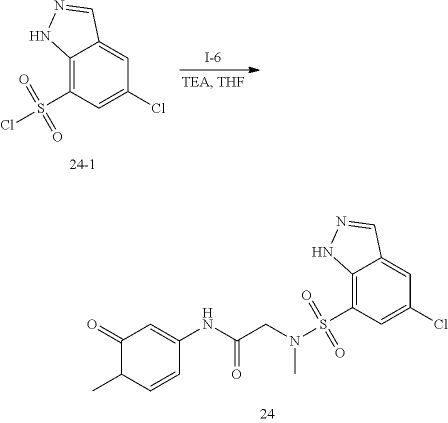

To a mixture of I-6 (0.101 g, 0.434 mmol) in tetrahydrofuran (2 mL) was added triethylamine (0.242 mL, 1.74 mmol) at 0° C., followed by 24-1 (0.194 g, 0.772 mmol). The mixture was stirred at 25° C. for 1 hour. The reaction mixture was concentrated under reduced pressure to give a residue and purified by prep-HPLC (column: 3_Phenomenex Luna C18 75*30 mm*3 urn; mobile phase: [water (0.05% HCl)-ACN]; B %: 20%-40%, 6.5 min) to give 2-(5-chloro-N-methyl-1H-indazole-7-sulfonamido)-N-(1-methyl-2-oxo-1,2-dihydropyridin-4-yl)acetamide (24) (0.028 g, 15.100 yield) as a yellow solid.

LCMS for Example 24: m/z 410.2 [M+H]⁺

¹H-NMR for Example 24: (DMSO-d6, 400 MHz) δ 13.47 (br. s, 1H), 10-9 (br. s, 1H), 8.33-8.17 (m, 2H), 7.74 (d, J=2.0 Hz, 1H), 7.57 (d, J=7.2 Hz, 1H), 6.57 (d, J=2.4 Hz, 1H), 6.27 (dd, (=2.-, 7.2 Hz, 1H), 4.23-4.18 (m, 2H), 3.33 (s, 3H), 2.90 (s, 3H)

The following compounds were synthesized in a similar manner as 24 using the reactants indicated.

| | | LCMS | |
|---|---|---|---|
| Example | Reactant | m/z | ¹H-NMR |
| 103 | I-16 | 450.1 | (DMSO-d₆, 400 MHz) δ |
| | | | 13.58 (br. s, 1H) −9.77 (s, |
| | | | 1H), 8.33-8.22 (m, 2H), |
| | | | 7.76 (d, −= 2.0 Hz, 1H), |
| | | | 6.95-6.83 (m, 2H), 6.62 |
| | | | (d, J = 8.4 Hz, 1H), 4.21 |
| | | | (t, J = 4.0 Hz, 2H), 4.09 |
| | | | (s, 2H), 3.16 (t, J = 4.0 Hz, |
| | | | 2H), 2.86 (s, 3H), 2.77 (s, 3H) |
| 166 | I-35 | 520.0 | (DMSO-d6, 400 MHz): δ |
| | | | 13.49 (br. s, 1H), 9.83 |
| | | | (br. s, 1H), 8.28 (s, 1H), |
| | | | 8.24 (s, 1H) −7.74 (s, 1H), |
| | | | 6.99-6.92 (m, 1H), 6.91- |
| | | | 6.84 (m, 1H), 6.82- |
| | | | 6.74 (m, 1H), 4.15-4.10 |
| | | | (m, 2H), 4.08 (s, 2H), |
| | | | 3.92 (dd, J = 3.6-11.2 |
| | | | Hz, 2H), 3.86-3.78 (m, |
| | | | 1H), 3.46-3.41 (m, 2H), |
| | | | 3.21-3.13 (m, 2H) −2.85 |
| | | | (s, 3H), 1.72-1.61 (m, 2H), |
| | | | 1.60-1.51 (m, 2H) |

The following compounds were synthesized in a similar manner as 24 starting from 1-40.

| | | LCMS | |
|---|---|---|---|
| Example | Reactant | m/z | ¹H-NMR |
| 150 | I-6 | 428.0 | (MeOD, 400 MHZ): δ |
| | | | 8.02 (d, J = 2.0 Hz, 1H), 7.86 |
| | | | (d, J = 2.0 Hz, 1H), 7.54 |
| | | | (d, J = 7.6 Hz, 1H), 6.76 |
| | | | (d, J = 2.4 Hz, 1H), 6.52 |
| | | | (dd, J = 7.6, 2.4 Hz, 1H), |
| | | | 4.22 (s, 2H), 3.49 (s, 3H), |
| | | | 2.99 (s, 3H) |
| 165 | I-35 | 538.2 | (DMSO-d6, 400 MHz): δ |
| | | | 13.05 (s, 1H), 9.74 (s, |
| | | | 1H), 8.25 (d, J = 1.6 Hz, |
| | | | 1H), 7.84 (d, J = 2.0 Hz, |
| | | | 1H), 6.89 (d, J −= 2.4 Hz, |
| | | | 1H), 6.86-6.82 (m, 1H), |
| | | | 6.80-6.75 (m, 1H), 4.15- |
| | | | 4.08 (m, 4H), 3.95- |
| | | | 3.88 (m, 2H), 3.86-3.78 |
| | | | (m, 1H), 3.47-3.40 (m, |
| | | | 2H), 3.21-3.14 (m, 2H) |
| | | | −2.88 (s, 3H), 1.73-1.61 |
| | | | (m, 2H), 1.59-1.51 (m, 2H) |

### Synthesis of (S)-1-((5-methyl-1H-indazol-7-yl)sulfonyl)-N-(1-methyl-2-oxo-1,2-dihydropyridin-4-yl)azetidine-2-carboxamide (Example 27)

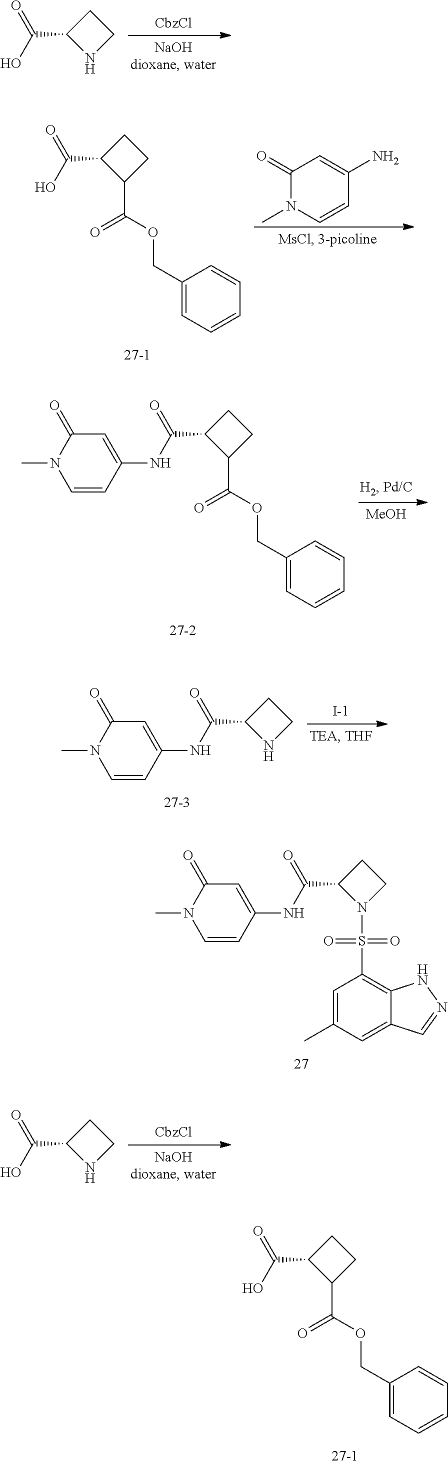

To a solution of sodium hydroxide (0.396 g, 9.89 mmol) in water (5 mL) and dioxane (2 mL) was added (S)-azetidine-2-carboxylic acid (0.500 g, 4.95 mmol) and CbzCl (0.928 g, 5.44 mmol) at 0° C. The mixture was stirred for 12 hours at 20° C. then poured into 1N hydrochloric acid (100 mL) and extracted with ethyl acetate (50 mL*3). The combined organic phase was washed with brine (50 mL), dried over anhydrous sodium sulfate, filtered, and the filtrate was concentrated under reduced pressure to afford (S)-1-((benzyloxy)carbonyl)azetidine-2-carboxylic acid (27-1) (1.00 g, 86% yield) as colorless gum. No further purification was performed.

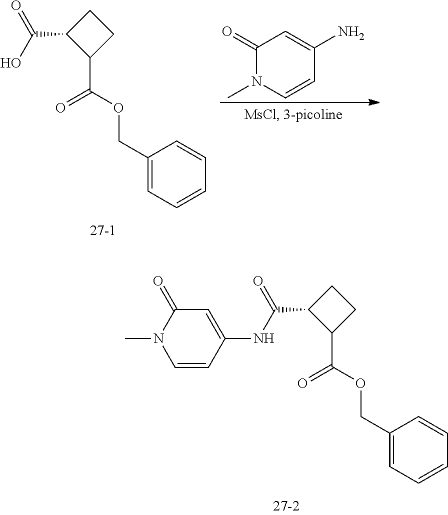

To a solution of 27-1 (0.430 g, 1.83 mmol) and 4-amino-1-methylpyridin-2(1H)-one (0.150 g, 1.21 mmol) in 3-picoline (2 mL) was added methanesulfonyl chloride (0.420 g, 3.67 mmol) drop wise at 0° C. The mixture was stirred for 2 hours at 0-20° C. then concentrated under reduced pressure. The residue was purified by reverse phase flash chromatography (0.1% ammonium hydroxide, acetonitrile (0-30%)) to afford (S)-benzyl 2-((1-methyl-2-oxo-1, 2-dihydropyridin-4-yl)carbamoyl) azetidine-1-carboxylate (27-1) (0.400 g, 94.5% yield) as a yellow solid.

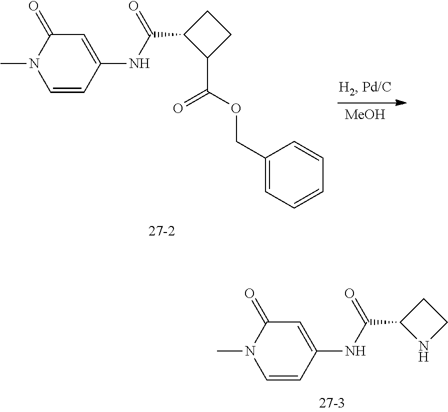 [SMILES: "MeOH" ; Cn1ccc(NC(=O)[C@@H]2CCC2C(=O)OCc2ccccc2)cc1=O ; Cn1ccc(NC(=O)[C@@H]2CCN2)cc1=O]

A solution of 27-2 (0.400 g, 1.2 mmol) in methanol (10 mL) was degassed with nitrogen three times, then Pd/C (50 mg, 10% on charcoal) was added in one portion. The mixture was degassed with hydrogen three times and stirred for 22 hours at 20° C. under a hydrogen atmosphere (15 psi). The mixture was filtered and the filtrate was concentrated under reduced pressure to afford (S)—N-(1-methyl-2-oxo-1, 2-dihydropyridin-4-yl)azetidine-2-carboxamide (27-3) (0.180 g, 68.2% yield) as a white solid. No further purification was performed.

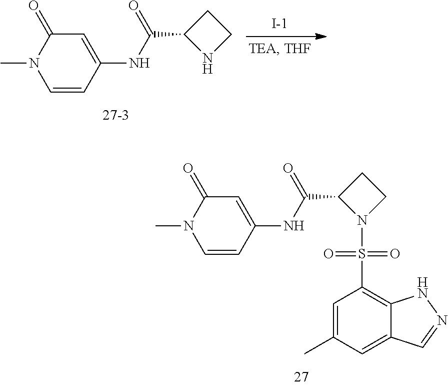 [SMILES: Cn1ccc(NC(=O)[C@@H]2CCN2)cc1=O ; [I-] ; Cc1cc(S(=O)(=O)N2CC[C@H]2C(=O)Nc2ccn(C)c(=O)c2)c2[nH]ncc2c1]

To a solution of 27-3 (0.050 g, 0.24 mmol) and triethylamine (0.100 mL, 0.723 mmol) in tetrahydrofuran (1 mL) was added I-1 (0.060 g, 0.216 mmol). The mixture was stirred for 3 hours at 20° C. then poured into water (5 mL), causing a solid to precipitate out. The formed solid was collected by filtration and the filter cake was triturated with acetonitrile (2 mL) to afford (S)-1-((5-methyl-1H-indazol-7-yl) sulfonyl)-N-(1-methyl-2-oxo-1, 2-dihydropyridin-4-yl) azetidine-2-carboxamide (27) (0.040 g, 41.4% yield) as a white solid.

LCMS of 27: m/z 402.1 [M+H]⁺

¹H-NMR of 27: (CDCl₃, 400 MHz) δ 11.36 (br. s, 1H), 9.05 (br. s, 1H), 8.17 (s, 1H), 7.91 (s, 1H)-7.68 (s, 1H), 7.29-7.27 (m, 1H), 6.84 (d, J-=2.0 Hz, 1H), 6.66-6.64 (m, 1H), 4.58-4.56 (m, 1H), 3.85-3.67 (m, 2H), 3.55 (s, 3H)-2.57 (s, 3H), 2.51-2.41 (m, 1H), 2.39-2.24 (m, 1H)

The following compound was synthesized in a similar manner as 27 using the reactant indicated.

| Compound | | LCMS | |
|---|---|---|---|
| No. | Reactant | m/z | ¹H-NMR |
| 39 | 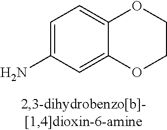 [SMILES: Nc1ccc2c(c1)OCCO2] | 429.1 | (CD₃OD, 400 MHz): δ 8.15 (s, 1H), 7.94 (d, J = 1.2 Hz, 1H), 7.77 (d, J = 1.2 Hz, 1H), 7.20 (d, J = 2.4 Hz, 1H), 6.94 (dd, J = 2.4, 8.8 Hz, 1H), 6.78 (d, J = 8.4 Hz, 1H), 4.79 (t, J- = 8.4 Hz, 1H), 4.26-4.21 (m, 4H), 3.94- 3.86 (m, 1H), 3.71-3.65 (m, 1H)- 2.53 (s, 3H), 2.42-2.34 (m, 2H) |
| 40 | 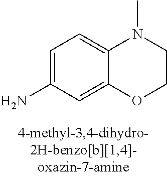 | 442.1 | (CD₃OD, 400 MHz) δ 8.15 (s, 1H), 7.94 (s, 1H), 7.77 (s, 1H), 7.02 (d, J = 2.4 Hz, 1H), 6.97 (dd, J = 8.8, 2.4 Hz, 1H), 6.67 (d, J = 8.8 Hz, 1H), 4.78 (t, J- = 8.4 Hz, 1H), 4.31-4.25 (m, 2H), 3.90 (q, J- = 8.8 Hz, 1H), 3.72-3.63 (m, 1H), 3.25-3.18 (m, 2H), 2.86 (s, 3H)- 2.53 (s, 3H), 2.42-2.31 (m, 2H) |
| 41 | 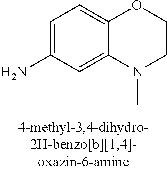 | 442.1 | (CD₃OD, 400 MHz): δ 8.16 (s, 1H), 7.94 (s, 1H), 7.78 (d, J = 1.2 Hz, 1H), 6.99 (d, J = 2.4 Hz, 1H), 6.75 (dd, J = 8.4, 2.4 Hz, 1H), 6.63 (d, J = 8.4 Hz, 1H), 4.78 (t, J− = 8.4 Hz, 1H), 4.26-4.22 (m, 2H), 3.94- 3.87 (m, 1H), 3.74-3.66 (m, 1H), 3.28-3.23 (m, 2H), 2.89 (s, 3H)- 2.53 (s, 3H), 2.42-2.33 (m, 2H) |
| 91 | 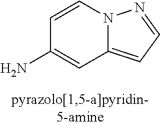 | 411.3 | (CDCl₃, 400 MHz) δ 8.92 (s, 1H), 8.44 (d, J = 7.2 Hz, 1H), 8.18 (s, 1H)-8.04 (s, 1H), 7.95-7.92 (m, 2H)- 7.92 (s, 1H), 6.83-6.81 (m, 1H), 6.48 (d, J- = 1.2 Hz, 1H), 4.53-4.49 (m, 1H), 3.91-3.86 (m, 1H), 3.70 (q, J = 8.4 Hz, 1H)-2.59 (s, 3H), 2.54-2.43 (m, 1H), 2.38-2.25 (m, 1H) |

The following compounds were synthesized in a similar manner as 27 using the reactant indicated below in replace of I-1.

| Compound No. | Reactant | LCMS m/z | ¹H-NMR |
|---|---|---|---|
| 148 | I-40 | 440.0 | (DMSO-d₆, 400MHz): δ 13.18 (br. S, 1H), |
| | | | 10.18 (br. S, 1H), 8.34 (s, 1H), 7.92 (s, 1H), |
| | | | 7.63 (d, J = 7.2 Hz, 1H), 6.75 (s, 1H), 6.35 |
| | | | (dd, J = 7.6, 2.0 Hz, 1H), 4.79 (t, J = 8.0 Hz, |
| | | | 1H), 3.84-3.79 (m, 2H), 3.36 (s, 3H), 2.34- |
| | | | 2.28 (m, 2H) |
| 149 | 24-1 | 422.0 | (DMSO-d6, 400 MHz) δ 13.66 (br. S, 1H), |
| | | | 10.22 (br. S, 1H), 8.32 (d, J = 2.0 Hz, 2H), |
| | | | 7.63 (d, J = 2.0 Hz, 1H), 7.63 (d, J = 7.6 Hz, |
| | | | 1H), 6.79 (d, J = 2.4 Hz, 1H), 6.40 (s, 1H), |
| | | | 4.83-4.79 (m, 1H), 3.79-3.73 (m, 2H), |
| | | | 3.53 (s, 3H), 2.45-2.30 (m, 2H) |
| 178 | 176-2 | 436.0 | (DMSO-d₆, 400 MHz) δ 13.60 (br. s, 1H), |
| | | | 10.14 (br. s, 1H), 7.88 (s, 1H), 7.80 (d, J = |
| | | | 0.8 Hz, 1H), 7.63 (d, J = 7.6 Hz, 1H), 6.75 |
| | | | (d, J = 2.4 Hz, 1H), 6.36 (dd, J₁ = 2.4 Hz, |
| | | | J₂ = 7.4 Hz, 1H), 4.71 (t, J −= 8.4 Hz, 1H), |
| | | | 3.79-3.70 (m, 2H), 3.36 (s, 3H) −2.52 (s, |
| | | | 3H), 2.32-2.23 (m, 2H) |

### Synthesis of N-(3-chloro-4-methoxyphenyl)-2-((3-(methoxymethyl)-N,5-dimethyl-1H-indazole)-7-sulfonamido)acetamide (Example 28)

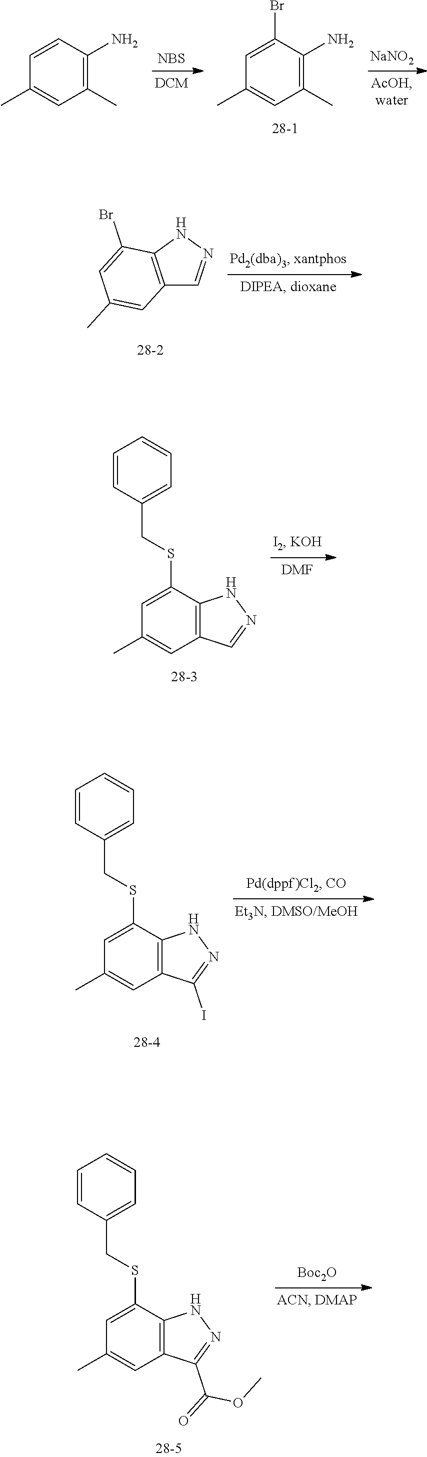

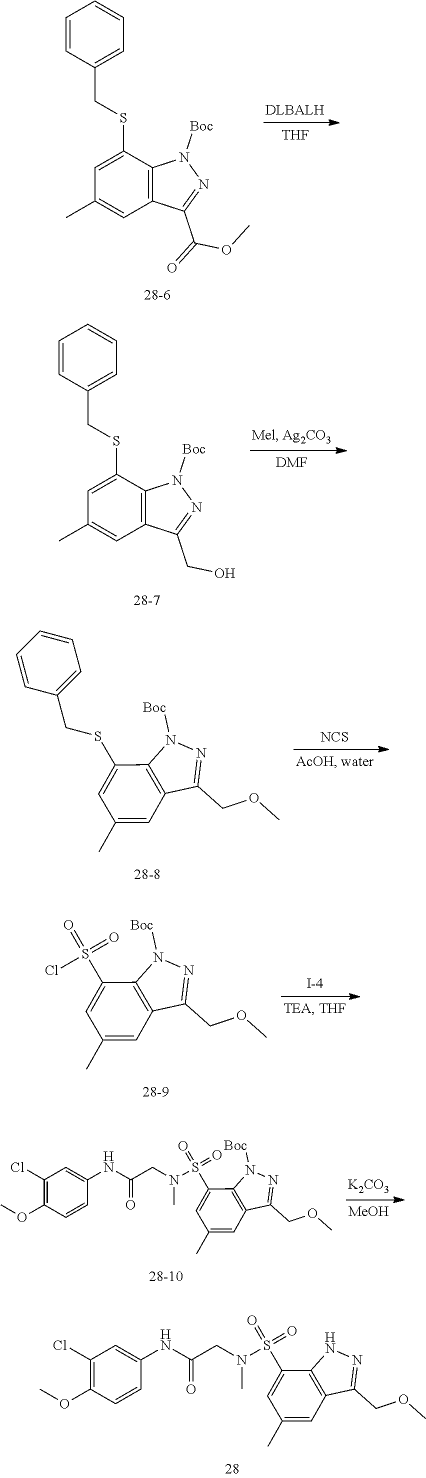

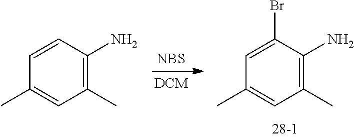

To a solution of 2,4-dimethylaniline (22.0 g, 182 mmol) in dichloromethane (200 mL) was added N-bromosuccinimide (37.0 g, 208 mmol) at 0° C. in portions, after the addition, the above reaction solution was stirred at 20° C. for 0.5 hour. The reaction mixture was diluted with dichloromethane (200 mL), washed with brine (100 mL*4), dried over anhydrous sodium sulfate, filtered, and the filtrate was concentrated under reduced pressure to afford 2-bromo-4,6-dimethylaniline (28-1) (37.5 g) as red oil which was used directly without further purification.

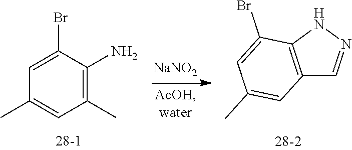

To a solution 28-1 (37.5 g, 187 mmol) in acetic acid (400 mL) was slowly added a solution of sodium nitrite (18.8 g, 272 mmol) in water (120 mL) at 10° C. The mixture was stirred at 20° C. for 2 hours. The reaction mixture was quenched with a 1N aqueous solution of sodium hydroxide to adjust the pH to around 12 and extracted with ethyl acetate (1 L*2). The combined organic phase was washed with brine (1 L*2), dried over anhydrous sodium sulfate, filtered, and the filtrate was concentrated under reduced pressure. The residue was purified by column chromatography (SiO₂, petroleum ether:ethyl acetate=1:0 to 20:1) to afford 7-bromo-5-methyl-1H-indazole (28-2) (4.80 g, 7.29% yield) as a red solid.

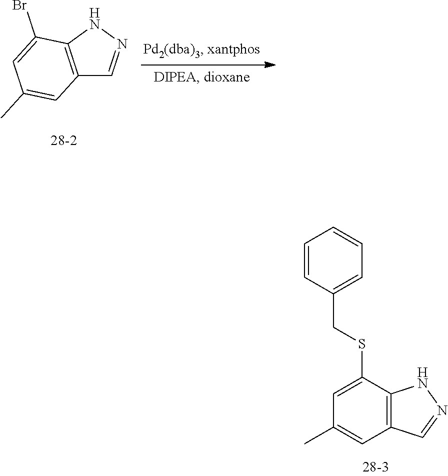 [SMILES: Cc1cc(Br)c2[nH]ncc2c1 ; Cc1cc(SCc2ccccc2)c2[nH]ncc2c1]

To a solution of 28-2 (0.800 g, 3.35 mmol) and N-ethyl-N-isopropylpropan-2-amine (2.00 mL, 11.5 mmol) in dioxane (8 mL) was added Pd₂(dba)₃ (0.690 g, 0.754 mmol) and xantphos (0.880 g, 1.52 mmol) under a nitrogen atmosphere. To this was added phenylmethanethiol (1.80 mL, 15.4 mmol) and the mixture was stirred at 105° C. for 12 hours. The mixture was cooled to room temperature, filtered, and the filter cake was rinsed with ethyl acetate (20 mL*3). The combined filtrate was concentrated under reduced pressure and the residue was purified by column chromatography (SiO₂, petroleum ether:ethyl acetate=20:1 to 5:1) to afford 7-(benzylthio)-5-methyl-1H-indazole (28-3) (0.800 g, 92.5% yield) as a red oil.

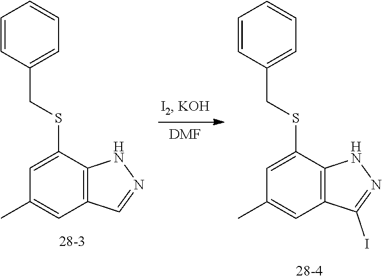

To a solution of 28-3 (3.60 g, 13.9 mmol) in dimethyl formamide (30 mL) was added potassium hydroxide (2.34 g, 41.8 mmol) then a solution of iodine (4.24 g, 16.7 mmol) in dimethyl formamide (10 mL) was added drop-wise to the mixture at 0° C. The reaction mixture was stirred at 25° C. for 2 hours. The reaction mixture was quenched with a saturated aqueous solution of sodium bisulfate (200 mL), and extracted with ethyl acetate (100 mL*2). The combined organic phase was washed with brine (100 mL*3), dried over anhydrous sodium sulfate, filtered, and the filtrate was concentrated under reduced pressure. The residue was purified by column chromatography (SiO₂, petroleum ether:ethyl acetate=20:1 to 10:1) to afford 7-(benzylthio)-3-iodo-5-methyl-1H-indazole (28-4) (3.90 g, 10.2 mmol, 73.0% yield) as a red solid.

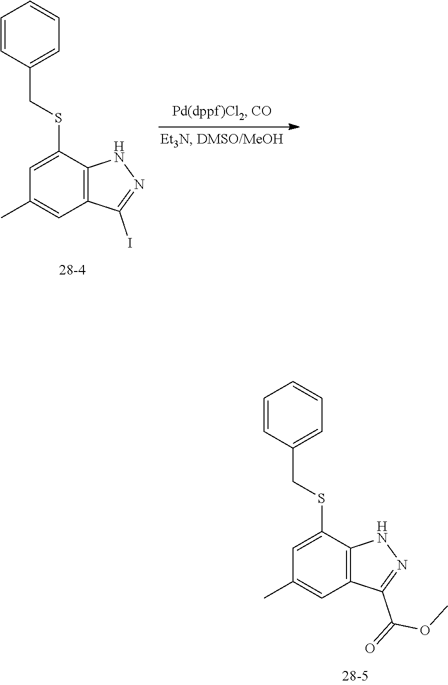

To a solution of 28-4 (1.00 g, 2.61 mmol) in a mixture of dimethylsulfoxide (10 mL) and methanol (10 mL) was added triethylamine (1.10 mL, 8.00 mmol) and Pd(dppf)Cl₂ (0.400 g, 0.547 mmol) under a nitrogen atmosphere. The suspension was degassed under reduced pressure, purged with carbon monoxide several times, and then stirred at 80° C. for 12 hours under a carbon monoxide (50 psi) atmosphere. The reaction mixture was diluted with ethyl acetate (100 mL) and washed with brine (100 ml*3). The organic phase was dried over anhydrous sodium sulfate, filtered, and the filtrate was concentrated under reduced pressure. The residue was purified by column chromatography (SiO₂, petroleum ether:ethyl acetate=10:1 to 4:1) to afford methyl 7-(benzylthio)-5-methyl-1H-indazole-3-carboxylate (28-5) (0.700 g, 85.3% yield) as a white solid.

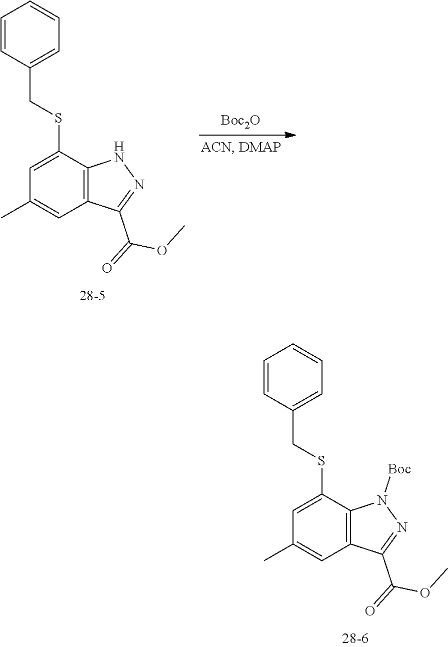

To a solution of methyl 28-5 (2.20 g, 6.97 mmol) in a mixture of acetonitrile (22 mL) and dimethyl formamide (2 mL) was added N,N-4-dimethylaminopyridine (0.085 g, 0.697 mmol) and triethylamine (1.94 mL, 13.9 mmol) then Boc₂O (3.20 mL, 13.9 mmol) was added drop wise to the mixture. The mixture was stirred at 25° C. for 3 hours then poured into ice water (200 mL) and extracted with ethyl acetate (50 mL*3). The combined organic layers were washed with brine/1N hydrochloric acid=5/1 (v/v, 50 mL), dried over anhydrous sodium sulfate, filtered, and the filtrate was concentrated under reduced pressure. The residue was purified by column chromatography (SiO₂, petroleum ether/ethyl acetate=40/1 to 10/1) to afford 1-tert-butyl 3-methyl 7-(benzylthio)-5-methyl-1H-indazole-1,3-dicarboxylate (28-6) (1.10 g, 2.63 mmol, 37.7% yield) as a yellow oil.

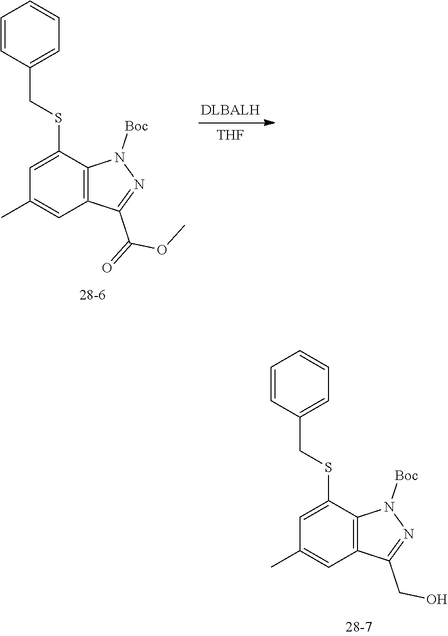

To a solution of 28-6 (1.10 g, 2.64 mmol) in tetrahydrofuran (12 mL) was added a solution of DIBAL-H (1 M, 13.2 mL, in tetrahydrofuran) at −78° C. under nitrogen atmosphere. The mixture was stirred at −78° C. for 2 hours then dropped into 1N hydrochloric acid (100 mL) and extracted with ethyl acetate (100 mL*3). The combined organic phase was washed with brine (100 mL*3), dried over anhydrous sodium sulfate, filtered, and the filtrate was concentrated under reduced pressure. The residue was purified by column chromatography (SiO₂, petroleum ether/ethyl acetate=15/1 to 4/1) to afford tert-butyl 7-(benzylthio)-3-(hydroxymethyl)-5-methyl-1H-indazole-1-carboxylate (28-7) (0.500 g, 48.1% yield) as a yellow oil.

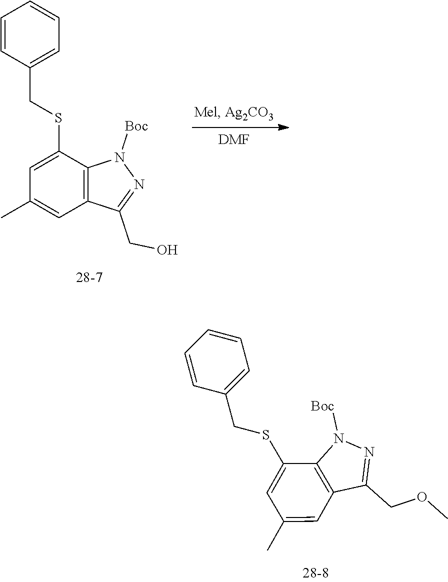

To a solution of 28-7 (0.700 g, 1.82 mmol) and silver carbonate (1.26 g, 4.55 mmol) in dimethyl formamide (7 mL) was added iodomethane (0.227 mL, 3.64 mmol) at 20° C. The mixture was stirred at 20° C. for 12 hours then filtered with Celite. The filtrate was diluted with ethyl acetate (50 mL), washed with brine (50 mL*3), dried over anhydrous sodium sulfate, filtered, and the filtrate was concentrated under reduced pressure. The residue was purified by column chromatography (SiO₂, petroleum ether/ethyl acetate=15/1 to 1/1) to afford tert-butyl 7-(benzylthio)-3-(methoxymethyl)-5-methyl-1H-indazole-1-carboxylate (28-8) (0.310 g, 39.3% yield) as a yellow oil.

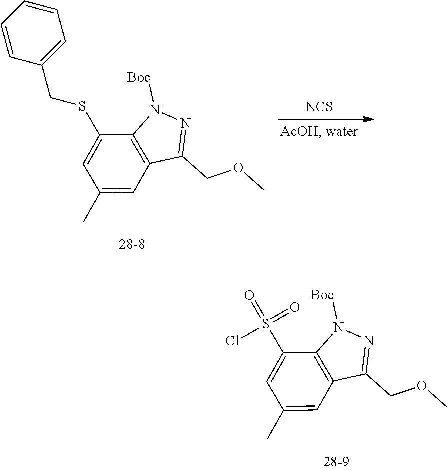 [SMILES: COCc1nn(C(=O)OC(C)(C)C)c2c(SCc3ccccc3)cc(C)cc12 ; "NCS" ; COCc1nn(C(=O)OC(C)(C)C)c2c(S(=O)(=O)Cl)cc(C)cc12]

To a solution of 28-8 (0.100 g, 0.231 mmol) in acetic acid (1 mL) and water (0.3 mL) was added NCS (0.123 g, 0.923 mmol) at 0° C. The mixture was stirred at 0-25° C. for 4 hours then poured into water (10 mL) and extracted with ethyl acetate (10 mL*3). The combined organic layers were washed with brine (20 mL), dried over anhydrous sodium sulfate, filtered, and the filtrate was concentrated under reduced pressure to afford tert-butyl 7-(chlorosulfonyl)-3-(methoxymethyl)-5-methyl-1H-indazole-1-carboxylate (28-9) (0.085 g, 59.7% yield) as a colorless oil.

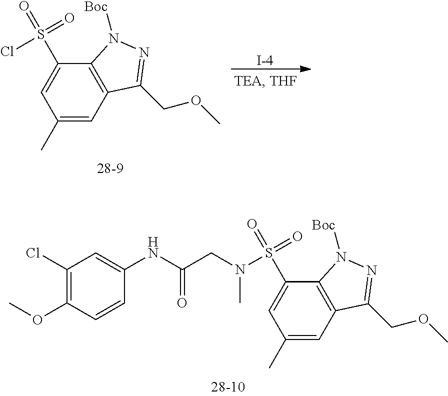

To a solution of 28-9 (0.060 g, 0.227 mmol) in tetrahydrofuran (1 mL) was added triethylamine (0.126 mL, 0.907 mmol), then I-4 (0.085 g, 0.227 mmol) was added to the mixture at 0° C. The mixture was stirred at 0° C. for 0.5 hour then poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was purified by prep-TLC (SiO₂, petroleum ether:ethyl acetate=1:1) to afford tert-butyl 7-(N-(2-((3-chloro-4-methoxyphenyl)amino)-2-oxoethyl)-N-methylsulfamoyl)-3-(methoxymethyl)-5-methyl-1H-indazole-1-carboxylate (28-10) (0.050 g, 27.6% yield) as yellow oil.

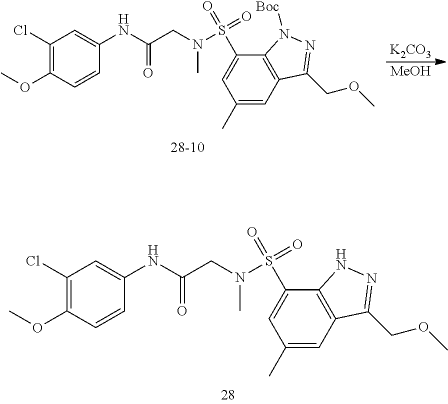

To a solution of 28-10 (0.020 g, 0.030 mmol) in methanol (0.5 mL) was added potassium carbonate (0.008 g, 0.061 mmol) at 0° C. The mixture was stirred at 0° C. for 0.5 hour, then poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was purified by prep-HPLC (column: Phenomenex Gemini-NX C18 75*30 mm*3 um; mobile phase: [water (0.1% TFA)-ACN]; B %: 45%-55%, 7 min) twice to afford N-(3-chloro-4-methoxyphenyl)-2-(3-(methoxymethyl)-N,5-dimethyl-1H-indazole-7-sulfonamido)acetamide (28) (0.003 g, 20.6% yield) as a brown solid.

LCMS of 28: m/z 467.1 [M+H]⁺

¹H-NMR of 28: (CDCl₃, 400 MHz): δ 8.08 (br. s, 1H)-7.98 (s, 1H), 7.67-7.61 (m, 2H), 7.42 (dd, J=8.8, 2.4 Hz, 1H), 6.92 (d, J=8.8 Hz, 1H), 4.87 (s, 2H), 3.91 (s, 3H), 3.87 (s, 2H), 3.47 (s, 3H), 2.91 (s, 3H), 2.57 (s, 3H)

### Synthesis of 2-((N,5-dimethyl-1H-indazole)-7-sulfonamido)-N-(4-methyl-3,4-dihydro-2H-benzo[b][1,4]oxazin-7-yl)acetamide (Example 30)

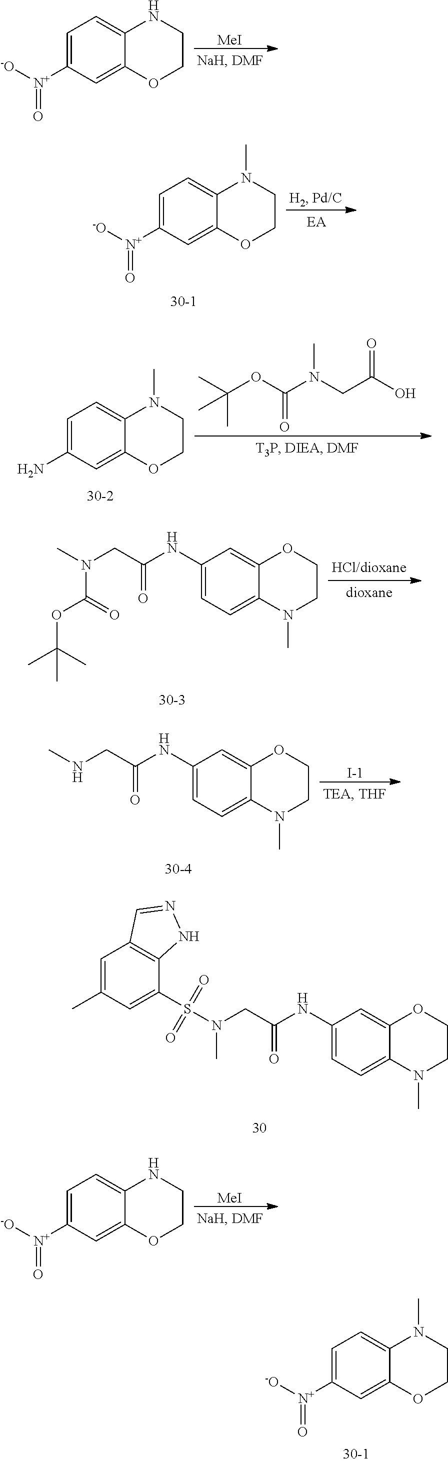

To a solution of 7-nitro-3,4-dihydro-2H-benzo[b][1,4]oxazine (0.100 g, 0.555 mmol) in dimethyl formamide (1 mL) was added iodomethane (0.052 mL, 0.833 mmol) and sodium hydride (0.044 g, 1.11 mmol, 60% purity in mineral oil) at 0° C. The mixture was stirred at 0° C. for 0.5 hour. The mixture was poured into water (2 mL), causing a solid to precipitate out. The formed solid was collected by filtration and the filter cake was dissolved with ethyl acetate (100 mL), dried over anhydrous sodium sulfate, filtered, and the filtrate was concentrated under reduced pressure to afford 4-methyl-7-nitro-3,4-dihydro-2H-benzo[b][1,4]oxazine (30-1) (0.100 g, 92.8% yield) as a yellow solid.

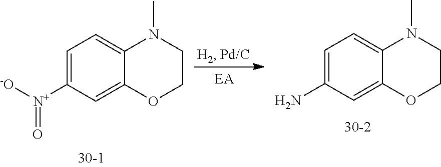

A solution of 30-1 (0.530 g, 2.73 mmol) in ethyl acetate (40 mL) was degassed with nitrogen three times then Pd/C (0.053 g, 10% purity on charcoal, wet) was added. The reaction mixture was degassed and purged with hydrogen three times. The mixture was stirred at 25° C. for 2 hours under a hydrogen atmosphere (15 psi) then diluted with ethyl acetate (100 mL), filtered, and the filtrate was concentrated under reduced pressure to afford 4-methyl-3, 4-dihydro-2H-benzo[b][1,4]oxazin-7-amine (30-2) (0.430 g, 88.0% yield) as a black brown oil.

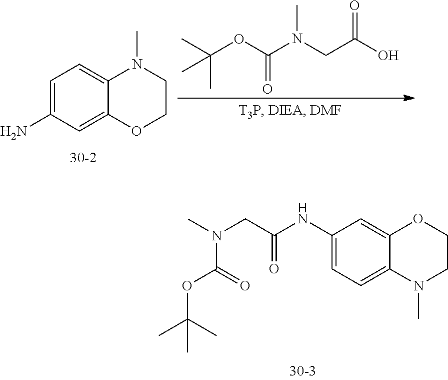

To a solution of 30-2 (0.200 g, 1.11 mmol) and 2-((tert-butoxycarbonyl)(methyl)amino)acetic acid (0.252 g, 1.33 mmol) in dimethyl formamide (2 mL) was added N-ethyl-N-isopropylpropan-2-amine (0.800 mL, 4.59 mmol), then T₃P (1.00 mL, 1.68 mmol, 50% purity in ethyl acetate) was added drop wise at 0° C. The mixture was stirred at 0° C. for 1 hour then poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was purified by flash silica gel chromatography (SiO₂, petroleum ether/ethyl acetate=1/1˜0/1) to afford tert-butylmethyl (2-((4-methyl-3, 4-dihydro-2H-benzo[b][1,4]oxazin-7-yl)amino)-2-oxoethyl)carbamate (30-3) (0.350 g, 90.7% yield) as a white solid.

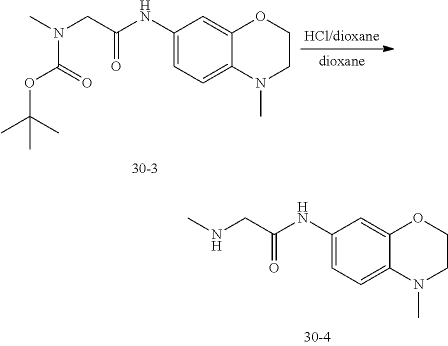 [SMILES: CN(CC(=O)Nc1ccc2c(c1)OCCN2C)C(=O)OC(C)(C)C ; CNCC(=O)Nc1ccc2c(c1)OCCN2C]

To a solution of 30-3 (0.350 g, 1.00 mmol) in dioxane (4 mL) was added a solution of HCl in dioxane (4 M, 1.00 mL, 4.00 mmol) drop wise. The mixture was stirred at 25° C. for 2 hours then concentrated under reduced pressure to afford N-(4-methyl-3,4-dihydro-2H-benzo[b][1,4]oxazin-7-yl)-2-(methylamino)acetamide dihydrochloride (30-4) (0.235 g, 76.1% yield) as a white solid.

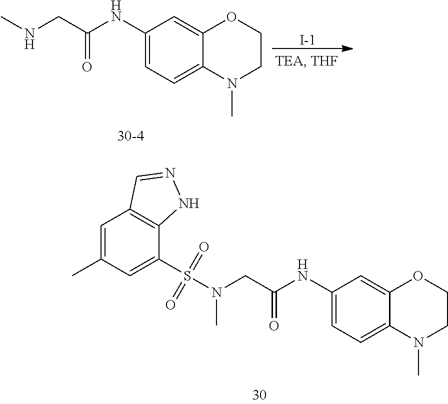 [SMILES: CNCC(=O)Nc1ccc2c(c1)OCCN2C ; [I-] ; Cc1cc(S(=O)(=O)N(C)CC(=O)Nc2ccc3c(c2)OCCN3C)c2[nH]ncc2c1]

To a solution of 30-4 (0.100 g, 0.324 mmol) in tetrahydrofuran (1 mL) was added triethylamine (0.181 mL, 1.30 mmol) at 0° C., then I-1 (0.120 g, 0.510 mmol) was added. The mixture was stirred at 0° C. for 1 hour then poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was purified by prep-HPLC (column: Waters Xbridge C18 150*50 mm*10 um; mobile phase: [water (10 mM NH₄HCO₃)-MeCN]; B %: 30%-60%, 10 min) to afford 2-(N,5-dimethyl-1H-indazole-7-sulfonamido)-N-(4-methyl-3,4-dihydro-2H-benzo[b][1,4]oxazin-7-yl)acetamide (30) (0.049 g, 34.0% yield) as a white solid.

LCMS of 30: m/z 430.1 [M+H]⁺

¹H-NMR of 30: (CDCl₃, 400 MHz) δ 11.36 (br. s, 1H), 8.13 (s, 1H), 7.93 (br. s, 1H), 7.84 (s, 1H)-7.64 (s, 1H), 7.04-6.95 (m, 2H), 6.63 (d, J-=8.4 Hz, 1H), 4.43-4.21 (m, 2H)-3.86 (s, 2H), 3.30-3.19 (m, 2H), 2.88 (s, 6H), 2.55 (s, 3H)

The following compound was synthesized in a similar manner as 30 using the reactant indicated.

| | | LCMS | |
|---|---|---|---|
| Example | Reactant | m/z | ¹H-NMR |
| 31 | 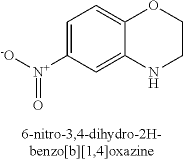 | 430.1 | (DMSO-d₆, 400 MHz): δ 13.29 (br. s, 1H), 9.76 (s, 1H), 8.19 (s, 1H), 7.90 (s, 1H), 7.64 (s, 1H), 6.89 (d, J = 2.0 Hz, 1H), 6.72 (dd, J = 8.4, 2.0 Hz, 1H), 6.57 (d, J- = 8.4 Hz, 1H), 4.21-4.15 (m, 2H)-4.02 (s, 2H), 3.24-3.19 (m, 2H), 2.81 (s, 3H), 2.78 (s, 3H), 2.46 (s, 3H) |

### Synthesis of 2-((5-bromo-N-methyl-1H-indazole)-7-sulfonamido)-N-(3-chloro-4-morpholinophenyl)acetamide (Example 32)

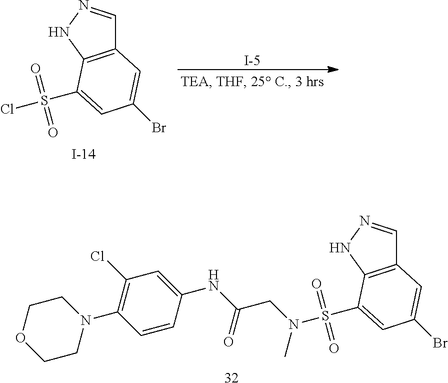

To a solution of I-5 as the di-hydrochloric salt (0.420 g, 1.18 mmol) and triethylamine (0.477 g, 4.71 mmol) in tetrahydrofuran (8 mL) was added I-14 (0.348 g, 1.18 mmol) in dichloromethane (5 mL) drop-wise. The mixture was stirred at 25° C. for 3 hours. The mixture was diluted with dichloromethane (20 mL), washed with brine (30 mL*2), dried over anhydrous sodium sulfate, filtered, and the filtrate was concentrated under reduced pressure. The residue was triturated with ethyl acetate (2 mL) to afford 2-(5-bromo-N-methyl-1H-indazole-7-sulfonamido)-N-(3-chloro-4-morpholinophenyl)acetamide (32) (0.450 g, 68.9% yield) as a white solid.

LCMS of 32: m/z 544.0 [M+H]⁺

¹H-NMR of 32: (DMSO-d6, 400 MHz): δ 13.52 (br. s, 1H), 10.11 (br. s, 1H), 8.39 (d, J=1.6 Hz, 1H), 8.27 (s, 1H), 7.83 (d, J=1.6 Hz, 1H), 7.62 (d, J=2.0 Hz, 1H), 7.32 (dd, J=8.4, 2.0 Hz, 1H), 7.11 (d, J=8.4 Hz, 1H)-4.15 (s, 2H), 3.75-3.69 (m, 4H), 2.93-2.87 (m, 7H)

The following compound was synthesized in a similar manner

| Example | Reactant | LCMS m/z | ¹H-NMR |
|---|---|---|---|
| 33 | I-6 | 454.0/456.0 | (DMSO-d6, 400 MHz): δ 13.45 (br. s, 1H), 10.16 |
| | | | (br. s, 1H), 8.38 (d, J = 1.6 Hz, 1H), 8.27 (s, 1H), |
| | | | 7.81 (d, J = 1.6 Hz, 1H), 7.57 (d, J = 7.2 Hz, 1H), |
| | | | 6.56 (d, J = 2.0 Hz, 1H), 6.27 (dd, J1 = 2.0 Hz, J2 = |
| | | | 7.2 Hz, 1H), 4.19 (s, 2H), 3.32 (s, 3H), 2.89 (s, 3H) |
| 93 | I-17 | 349.0 | (DMSO-d6 + D₂O, 400 MHZ): δ 8.27 (s, 1H), 7.95 |
| | | | (dd, J = 2.4 Hz , 8.8 Hz, 1H), 7.67 (dd, J = 2.4 Hz, |
| | | | 8.8 Hz, 1H), 7.56 (d, J = 7.6 Hz, 1H), 6.58 (d, J = |
| | | | 2.0 Hz, 1H), 6.29 (dd, J = 2.4 Hz, 7.2 Hz, 1H), 4.17 |
| | | | (s, 2H), 3.32 (s, 3H), 2.87 (s, 3H) |

The following compound was synthesized in a similar manner using I-19

| Example | Reactant | LCMS m/z | ¹H-NMR |
|---|---|---|---|
| 95 | I-6 | 408.0 | (400 MHZ, MeOH-d₄) δ 7.77 (d, J = 7.03 Hz, 2H), |
| | | | 7.56 (d, J = 7.40 Hz, 1H), 6.82 (d, J = 2.26 Hz, |
| | | | 1H), 6.57 (dd, J = 7.40, 2.26 Hz, 1H), 4.15 (s, 2H), |
| | | | 3.50 (s, 3H), 2.94 (s, 3H), 2.51 (s, 3H) |
| 101 | I-5 | 496.1 | (400 MHZ, −METHANOL-d4) δ 7.85-7.74 (m, |
| | | | 2H), 7.64 (d, J = 2.4 Hz, 1H), 7.37 (dd, J = 2.5, 8.7 |
| | | | Hz, 1H), 7.11 (d, J = 8.7 Hz, 1H) −4.13 (s, 2H), |
| | | | 3.90-3.78 (m, 4H), 3.09-2.98 (m, 4H), 2.95 (s, |
| | | | 3H), 2.52 (s, 3H) |
| 121 | I-35 | 518.2 | (400 MHZ, METHANOL-d4) −7.80 (s, 2H), 6.95- |
| | | | 6.90 (m, 2H), 6.81 (d, J = 8.6 Hz, 1H), 4-58 (br s, |
| | | | 2H), 4.22-4.18 (m, 2H), 4.08 (s, 2H), 4.04 (br d, |
| | | | J −= 4.4 Hz, 1H), 3.94-3.85 (m, 1H), 3.61-3.54 |
| | | | (m, 2H), 3.27 (d, J = 4.4 Hz, 1H), 2.92 (s, 3H) |
| | | | −2.52 (s, 3H), 1.86-1.77 (m, 2H), 1.74-1.67 (m, |
| | | | 2H) |
| 140 | I-37 | 490.2 | (400 MHZ, METHANOL-d4) δ 7.77 (s, 2H), 6.96 |
| | | | (d, J = 2.4 Hz, 1H), 6.86 (dd, J = 2.4, 8.6 Hz, 1H), |
| | | | 6.20 (d, J = 8.8 Hz, 1H), 4.89 (br s, 1H), 4.78 (t, J = |
| | | | 6.6 Hz, 2H) −4.58 (s, 1H), 4.56-4.49 (m, 1H), |
| | | | 4.35-4.32 (m, 2H) −4.06 (s, 2H), 3.18-3.15 (m, |
| | | | 2H), 2.89 (s, 3H), 2.50 (s, 3H) |

### Synthesis of N-(3-chloro-4-morpholinophenyl)-2-((5-cyclopropyl-N-methyl-1H-indazole)-7-sulfonamido)acetamide (Example 34)

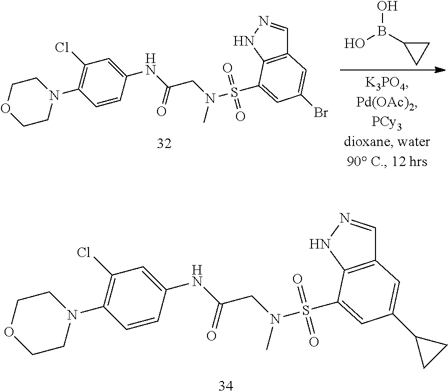

To a solution of 32 (0.100 g, 0.184 mmol) in water (0.6 mL) and dioxane (3 mL) was added cyclopropylboronic acid (0.250 g, 2.91 mmol), tricyclohexylphosphine (0.050 g, 0.178 mmol), potassium phosphate (1.00 g, 4.71 mmol) and Pd(OAc)₂ (0.050 g, 0.223 mmol) under nitrogen. The mixture was stirred at 90° C. for 12 hours under nitrogen atmosphere. After being cooled to room temperature, the mixture was diluted with methanol (30 mL) and filtered, the filtrate was concentrated under reduced pressure. The residue was purified by reversed phase HPLC (0.1% HCl aqueous/acetonitrile condition), followed by prep-HPLC (column: Waters Xbridge 150*25 mm*5 um; mobile phase: [water (10 mM NH₄HCO₃)-ACN]; B %: 37%-57%, 9 min) to afford N-(3-chloro-4-morpholinophenyl)-2-(5-cyclopropyl-N-methyl-1H-indazole-7-sulfonamido)acetamide (34) (0.011 g, 25.6% yield) as a yellow solid.

LCMS of 34: m/z 504.3 [M+H]⁺

¹H-NMR of 34: (DMSO-d6, 400 MHz): δ 13.21 (br s, 1H), 10.12 (br s, 1H), 8.17 (s, 1H), 7.79 (d, J=1.2 Hz, 1H), 7.68 (d, J=1.6 Hz, 1H), 7.52 (d, J=1.6 Hz, 1H), 7.36 (d, J=7.2 Hz, 1H), 7.12 (d, J=8.8 Hz, 1H)-4.08 (s, 2H), 3.75-3.70 (m, 4H), 2.93-2.88 (m, 4H), 2.83 (s, 3H), 2.11 (t, J-=5.2 Hz, 1H), 1.01-0.94 (m, 2H), 0.76-0.70 (m, 2H)

The following compound was synthesized in a similar manner as 34 using the reactant indicated.

| | | LCMS | |
|---|---|---|---|
| Example | Reactant | m/z | ¹H-NMR |
| 35 | 33 | 416.1 | (CDCl₃, 400 MHz) δ |
| | | | 11.97 (br. s, 1H), 9.05 (br. s, |
| | | | 1H), 8.12 (s, 1H), 7.75 |
| | | | (d, J = 0.8 Hz, 1H), 7.62 (s, |
| | | | 1H), 7.26-7.24 (m, 1H), |
| | | | 6.81 (J = 2.4 Hz, 1H), |
| | | | 6.68 (s, 1H), 3.99 (s, 2H), |
| | | | 3.53 (s, 3H) −2.87 (s, |
| | | | 3H), 2.11-2.06 (m, 1H), |
| | | | 1.09-1.03 (m, 2H), 0.80- |
| | | | 0.74 (m, 2H) |

### Synthesis of N-(1-methyl-2-oxo-1,2-dihydropyridin-4-yl)-2-((5-methyl-N-(oxetan-3-yl)-1H-indazole)-7-sulfonamido)acetamide (Example 38)

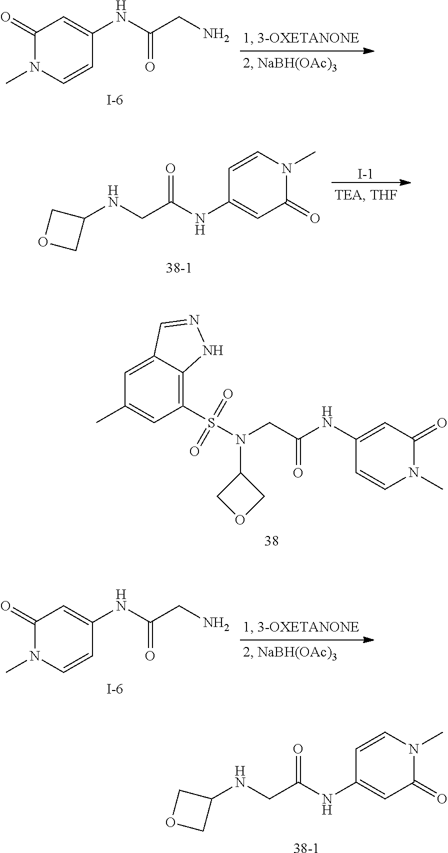

To a solution of I-6 (0.090 g, 0.409 mmol) in methanol (1 mL) was added sodium acetate (0.067 g, 0.819 mmol) and oxetan-3-one (0.038 g, 0.532 mmol). The mixture was stirred at 25° C. for 3 hours then sodium cyanoborohydride (0.039 g, 0.614 mmol) was added and the mixture was stirred at 25° C. for 12 hours. The reaction mixture was diluted with methanol (3 mL) and purified by reversed-phase HPLC (0.1% HCl condition) to afford 2-(isopropylamino)-N-(1-methyl-2-oxo-1,2-dihydropyridin-4-yl)acetamide hydrochloride (38-1) (0.050 g, 44.4% yield) as a yellow solid.

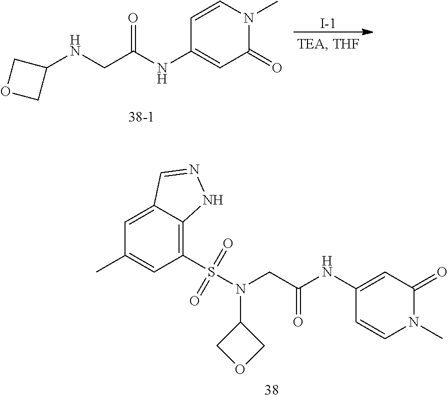

To a solution of 38-1 (0.025 g, 0.104 mmol) in pyridine (0.5 mL) was added I-1 (0.029 g, 0.125 mmol) at 25° C. The mixture was stirred for 10 hours at 50° C., then concentrated under reduced pressure. The residue was purified by prep-HPLC (column: Waters Xbridge C18 150*50 mm*10 um; mobile phase: [water (10 mM NH₄HCO₃)-MeCN]; B %: 10%-40%, 10 min) to afford N-(1-methyl-2-oxo-1,2-dihydropyridin-4-yl)-2-(5-methyl-N-(oxetan-3-yl)-1H-indazole-7-sulfonamido)acetamide (38) (0.018 g, 39.1% yield) as a white solid.

LCMS of 38: m/z 432.1 [M+H]⁺

¹H-NMR of 38: (CD₃OD, 400 MHz) δ 8.12 (s, 1H), 7.90 (s, 1H), 7.74 (s, 1H), 7.58 (d, J=7.6 Hz, 1H), 6.82 (d, J=2.0 Hz, 1H), 6.62 (dd, J=7.-, 2.4 Hz, 1H), 5.22-5.17 (m, 1H), 4.64 (t, J=7.6 Hz, 2H), 4.52 (t, J=6.8 Hz, 2H), 4.43 (s, 2H), 3.51 (s, 3H), 2.51 (s, 3H)

### Synthesis of N-(3-chloro-4-morpholinophenyl)-2-((N,2,5-trimethyl-1H-indole)-7-sulfonamido)acetamide (Example 42)

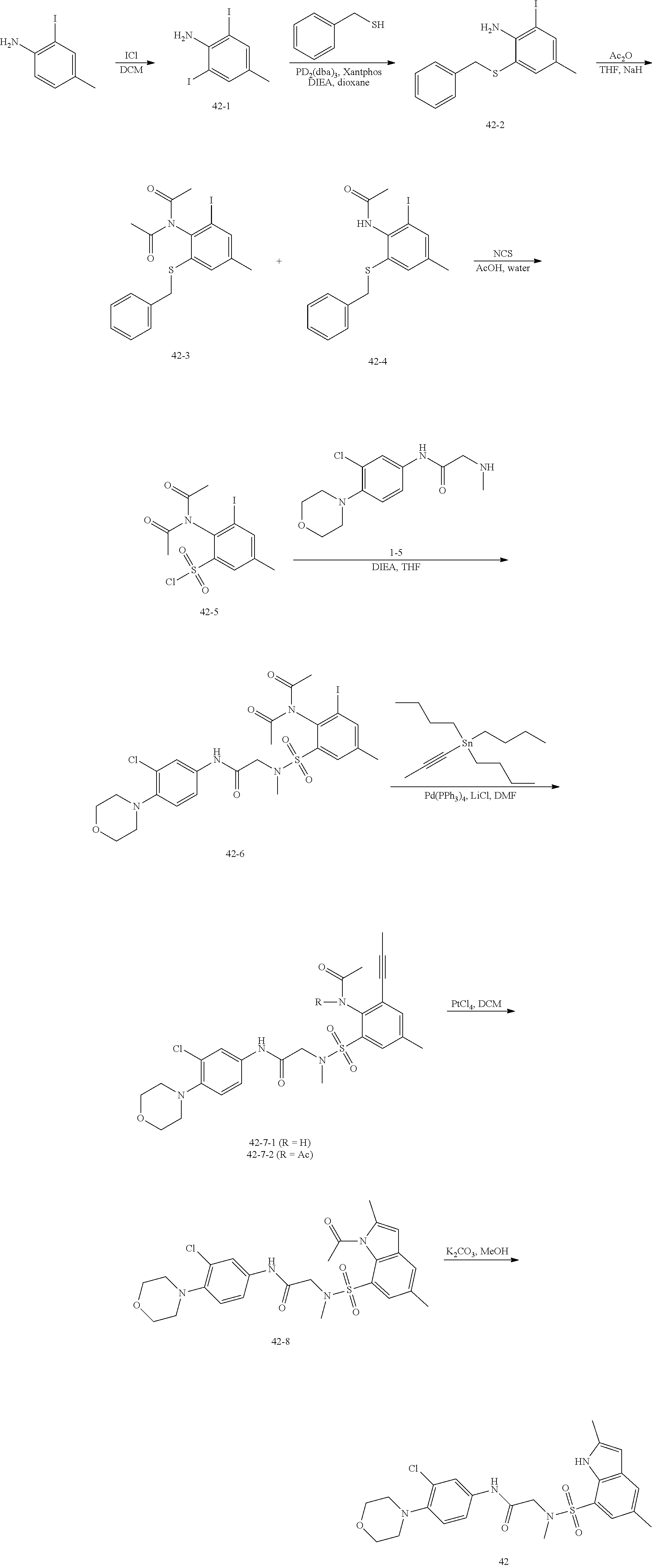

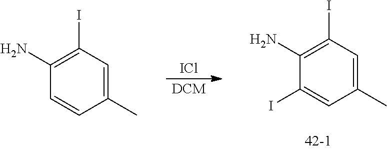

To a solution of 2-iodo-4-methylaniline (7.00 g, 30.0 mmol) in dichloromethane (70 mL) was added iodine monochloride (4.88 g, 30.0 mmol) drop-wise. The mixture was stirred for 16 hours at 20° C. The mixture was slowly poured into a cooled saturated aqueous solution of sodium sulfite (200 mL) and extracted with ethyl acetate (50 mL*3). The combined organic phase was washed with brine (50 mL), dried over anhydrous sodium sulfate, filtered, and then the filtrate was concentrated under reduced pressured. The residue was purified by column (SiO₂, petroleum ether to petroleum ether:ethyl acetate=100:1) to afford 2, 6-diiodo-4-methylaniline (42-1) (8.00 g, 68.3% yield) as white solid

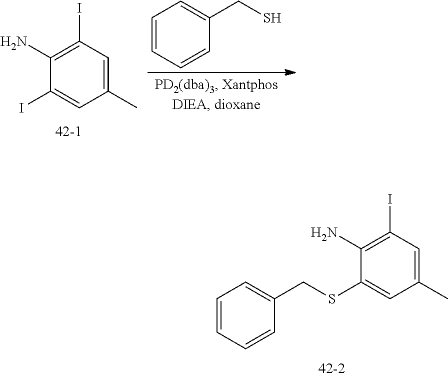

To a solution of 42-1 (6.00 g, 16.7 mmol), phenylmethanethiol (1.80 g, 14.5 mmol) and diisopropylethylamine (8.90 g, 68.9 mmol) in dioxane (100 mL) was added Pd₂(dba)₃ (0.800 g, 0.874 mmol) and Xantphos (1.00 g, 1.73 mmol). The mixture was degassed and purged with nitrogen three times, and then the mixture was stirred for 1.5 hours at 80° C. The mixture was filtered and the filtrate was concentrated under reduced pressured. The residue was purified by column (SiO₂, petroleum ether to petroleum ether:ethyl acetate) to afford 2-(benzylthio)-6-iodo-4-methylaniline (42-2) (5.60 g, 67.9% yield) as yellow gum.

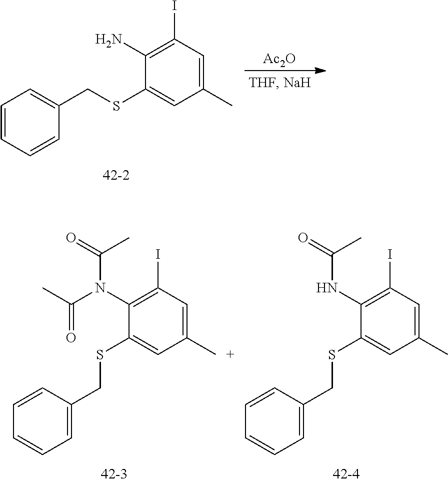

To a solution of 42-2 (0.40 g, 1.1 mmol) in tetrahydrofuran (5 mL) was added NaH (0.13 g, 3.2 mmol, 60% purity in mineral oil) and acetyl acetate (0.22 g, 2.1 mmol). The mixture was stirred for 60 hours at 70° C. then poured into ice water (10 mL) and extracted with ethyl acetate (20 mL*3). The combined organic phase was washed with brine (50 mL), dried over anhydrous sodium sulfate, filtered and the filtrate was concentrated under reduced pressure to afford a mixture of N-(2-(benzylthio)-6-iodo-4-methylphenyl)acetamide (42-3) (0.15 g, crude) and N-acetyl-N-(2-(benzylthio)-6-iodo-4-methylphenyl)acetamide (42-4) (0.35 g, crude) which were not separated but used directly in the following reaction.

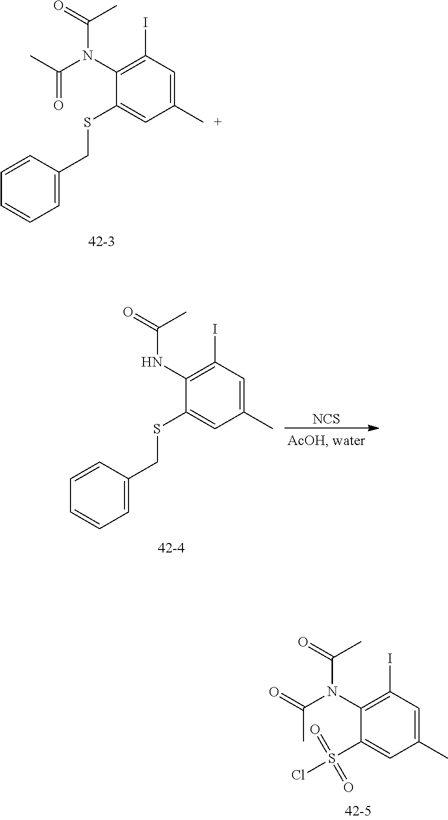

To a mixture of 42-3 (0.10 g, 0.25 mmol) and 42-4 (0.25 g, 0.57 mmol) in acetic acid (4 mL) and water (0.5 mL) was added NCS (0.42 g, 3.1 mmol) at 10° C. The mixture was stirred for 1 hour at 20° C. then poured into water (50 mL) and extracted with ethyl acetate (50 mL*2). The combined organic phase was washed with brine (50 mL*2), dried over anhydrous sodium sulfate, filtered, and the filtrate was concentrated under reduced pressure. The residue was purified by column (SiO₂, petroleum ether:ethyl acetate=20:1 to 3:1) to afford 2-(N-acetylacetamido)-3-iodo-5-methylbenzene sulfonyl chloride (42-5) (0.20 g, 0.43 mmol, 76.1% yield) as a yellow gum.

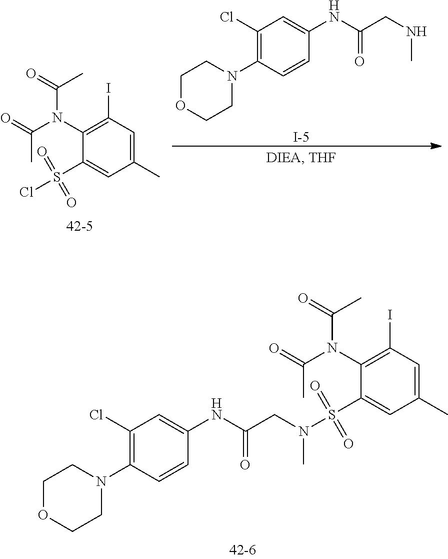

To a solution of I-5 (0.17 g, 0.48 mmol) as di-hydrochloric salt and triethylamine (0.20 g, 1.92 mmol) in dimethyl formamide (2 mL) was added a mixture of 42-5 (0.20 g, 0.48 mmol) in tetrahydrofuran (1 mL) at 0° C. The mixture was stirred for 1 hour at 20° C. then poured into water (10 mL) and extracted with ethyl acetate (20 mL*3). The combined organic phase was washed with brine (20 mL), dried over anhydrous sodium sulfate, filtered, and the filtrate was concentrated under reduced pressured. The residue was purified by prep-HPLC (column: Phenomenex Gemini-NX C18 75*30 mm*3 um; mobile phase: [water (0.1% TFA)-ACN]; B %: 55%-65%, 7 min) to afford N-acetyl-N-(2-(N-(2-((3-chloro-4-morpholinophenyl)amino)-2-oxoethyl)-N-methylsulfamoyl)-6-iodo-4-methylphenyl)acetamide (42-6) (0.12 g, 37.6% yield) as a white solid.

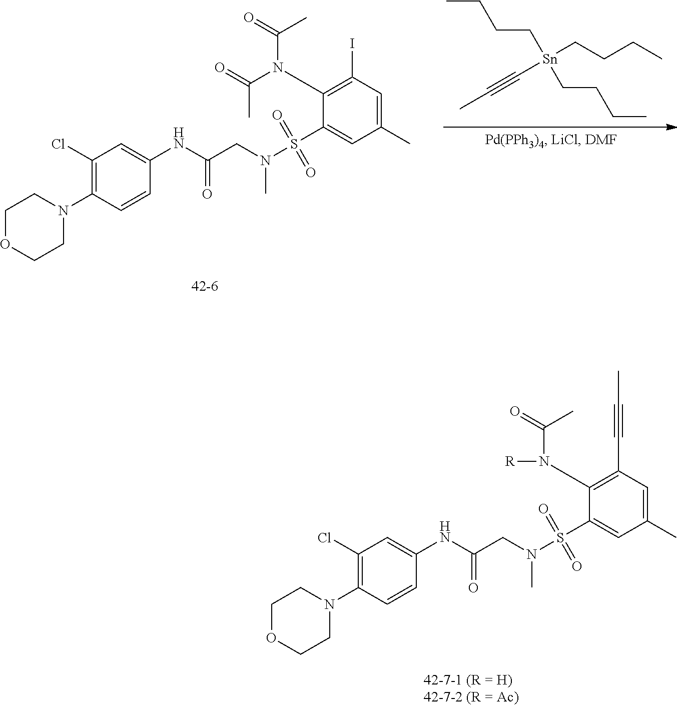

To a solution of 42-6 (0.10 g, 0.15 mmol), LiCl (0.02 g, 0.35 mmol) and tributyl(prop-1-yn-1-yl)stannane (0.10 g, 0.30 mmol) in dioxane (2 mL) was added Pd(PPh₃)₂Cl₂ (0.02 g, 0.02 mmol) under nitrogen atmosphere. The mixture was degassed and purged with nitrogen three times and stirred for 12 hours at 100° C. under nitrogen atmosphere. The mixture was filtered and concentrated under reduced pressure. The residue was purified by reversed phase flash chromatography (0.1% TFA in water, MeCN) to afford N-acetyl-N-(2-(N-(2-((3-chloro-4-morpholinophenyl)amino)-2-oxoethyl)-N-methylsulfamoyl)-4-methyl-6-(prop-1-yn-1-yl)phenyl)acetamide (42-7-2) (0.04 g35% yield) as yellow gum and 2-((2-acetamido-N,5-dimethyl-3-(prop-1-yn-1-yl)phenyl)sulfonamido)-N-(3-chloro-4-morpholinophenyl)acetamide (47-2-1) (0.06 g, 56% yield) as yellow gum.

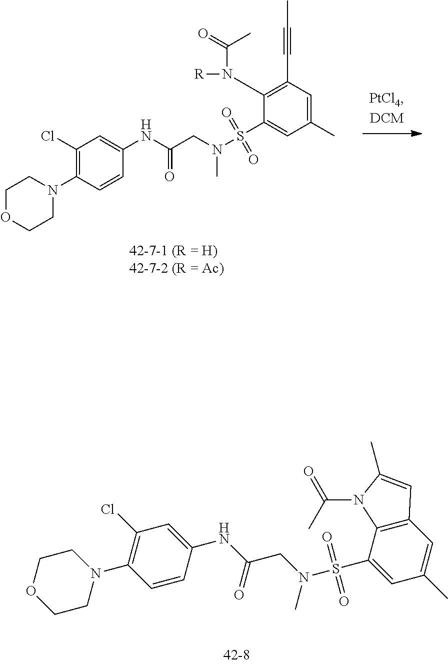

To a solution of 42-7-1 (0.06 g, 0.08 mol) in dichloromethane (1 mL) was added tetrachloroplatinum (0.003 g, 0.009 mmol). The mixture was stirred at 15° C. for 1 hour under an air atmosphere. The mixture was filtered and concentrated under reduced pressure. The residue was purified by reversed phase flash (0.01% TFA in water, MeCN) to afford 2-((1-acetyl-N, 2, 5-trimethyl-1H-indole)-7-sulfonamido)-N-(3-chloro-4-morpholino phenyl)acetamide (42-8) (0.04 g, 83.6% yield) as a white solid.

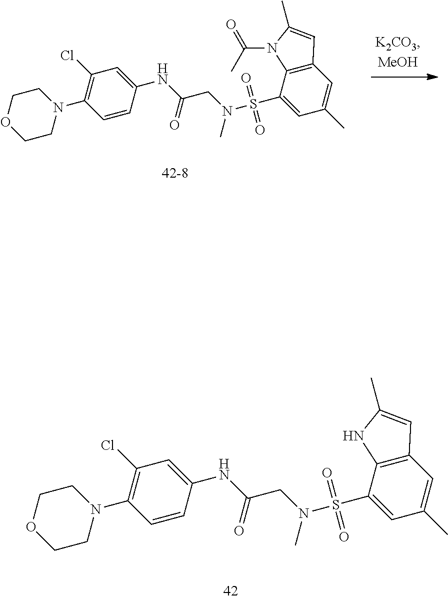 [SMILES: CC(=O)n1c(C)cc2cc(C)cc(S(=O)(=O)N(C)CC(=O)Nc3ccc(N4CCOCC4)c(Cl)c3)c21 ; Cc1cc(S(=O)(=O)N(C)CC(=O)Nc2ccc(N3CCOCC3)c(Cl)c2)c2[nH]c(C)cc2c1]

To a solution of 2-((1-acetyl-N, 2, 5-trimethyl-1H-indole)-7-sulfonamido)-N-(3-chloro-4-morpholinophenyl)acetamide (10) (0.04 g, 0.08 mmol) in methanol (1 mL) was added potassium carbonate (0.04 g, 0.03 mmol). The mixture was stirred for 1 hour at 20° C. The mixture was filtered and the filtrate was concentrated under reduced pressure.

The residue was purified by prep-HPLC (column: Phenomenex luna C18 150*25 mm*10 um; mobile phase: [water (0.05% HCl)-ACN] B %: 53%-83%, 10 min) to afford N-(3-chloro-4-morpholinophenyl)-2-((N,2,5-trimethyl-1H-indole)-7-sulfonamido)acetamide (T019W-006-49) (0.008 g, 0.16 mmol, 16% yield) as a yellow solid.

LCMS of 42: m/z 491.2 [M+H]⁺

¹H-NMR of 42: (CDCl₃, 400 MHz): δ 9.34 (br. s, 1H), 8.-8 (br. s, 1H), 8.02-7.77 (m, 2H)-7.58 (s, 1H), 7.51-7.40 (m, 1H), 7.30 (s, 1H)-6.25 (s, 1H), 4.22-4.11 (m, 4H)-3.87 (s, 2H), 3.55-3.41 (m, 4H), 2.89 (s, 3H), 2.49 (s, 6H).

The following compound was synthesized in a similar manner as 42 using the reactant indicated in replace of I-5.

| | | LCMS | |
|---|---|---|---|
| Example | Reactant | m/z | ¹H-NMR |
| 134 | I-6 | 403.3 | (DMSO-d₆, 400 MHz) δ |
| | | | 10.86 (s, 1H), 10.25 (s, |
| | | | 1H), 7.61 (d, J = 7.2 Hz, |
| | | | 1H), 7.53 (s, 1H), 7.22 (s, |
| | | | 1H), 6.73 (d, J = 2.0 Hz, |
| | | | 1H), 6.36 (dd, J = 2.0, 7.2 |
| | | | Hz, 1H), 6.21 (s, 1H), 4.03 |
| | | | (s, 2H), 3.35 (s, 3H), |
| | | | 2.77 (s, 3H), 2.42 (s, 3H), |
| | | | 2.40 (s, 3H) |

### Synthesis of 2-((4-fluoro-N,5-dimethyl-1H-indazole)-7-sulfonamido)-N-(1-methyl-2-oxo-1,2-dihydropyridin-4-yl)acetamide (Example 43)

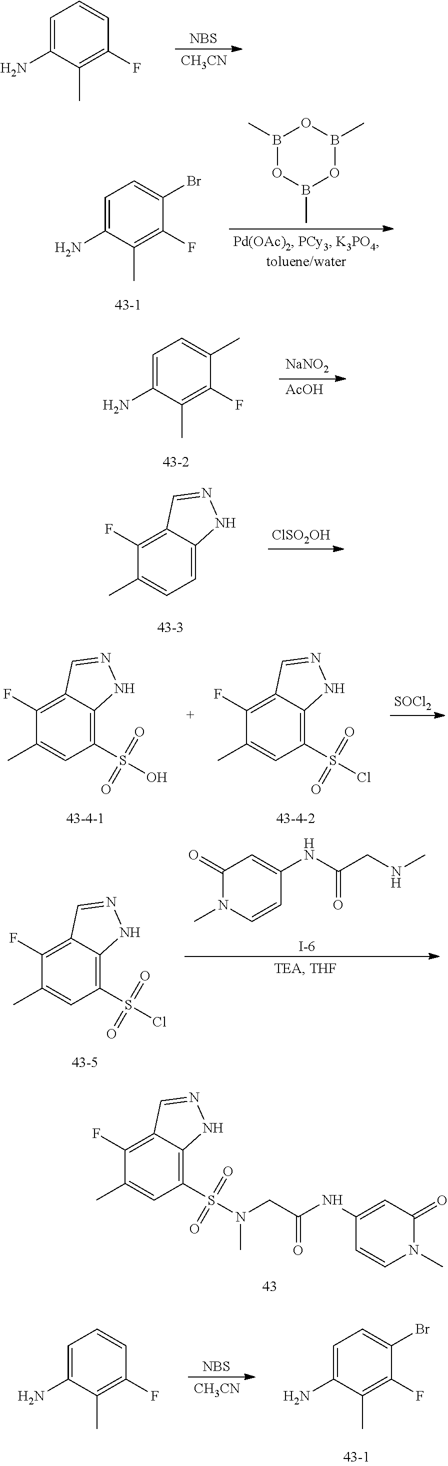

To a solution of 3-fluoro-2-methylaniline (8.00 g, 63.9 mmol) in acetonitrile (50 mL) was added 1-bromopyrrolidine-2,5-dione (12.5 g, 70.3 mmol) in five portions at 0° C. The mixture was warmed to 20° C. and stirred for 2 hours. The mixture was quenched with water (50 mL) and extracted with ethyl acetate (80 mL*4). The combined organic phase was washed with brine (80 mL), dried over anhydrous sodium sulfate, filtered, and the filtrate was concentrated under reduced pressure. The residue was purified by column chromatography on silica gel (petroleum ether:ethyl acetate=50:1, 10:1) to afford 4-bromo-3-fluoro-2-methylaniline (43-1) (12.0 g, 90.6% yield) as a brown solid.

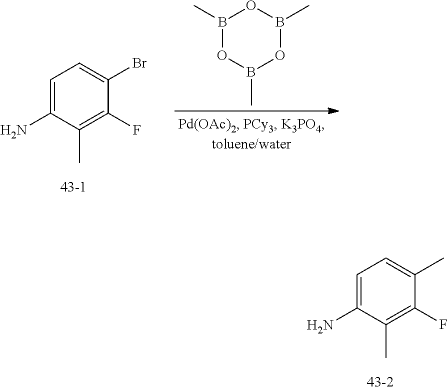

Compound 4-bromo-3-fluoro-2-methylaniline (43-1) (6.00 g, 29.4 mmol), 2,4,6-trimethyl-1,3,5,2,4,6-trioxatriborinane (16.4 mL, 58.8 mmol, 50% in tetrahydrofuran), tricyclohexylphosphine (1.65 g, 5.88 mmol) and potassium phosphate (18.7 g, 88.2 mmol) in a mixture of toluene (50 mL) and water (10 mL) was degassed and purged with nitrogen three times. To this was added Pd(OAc)₂ (0.66 g, 2.94 mmol) at 20° C. and the mixture was degassed and purged with nitrogen three times. The mixture was heated to 80° C. and stirred for 3 hours under nitrogen atmosphere then cooled to 20° C., water (50 mL) was added, then extracted with ethyl acetate (100 mL*4). The combined organic phase was washed with brine (50 mL), dried over anhydrous sodium sulfate, filtered, and the filtrate was concentrated under reduced pressure. The residue was purified by column chromatography on silica gel (petroleum ether:ethyl acetate=50:1, 10:1) to afford 3-fluoro-2, 4-dimethylaniline (43-2) (4.00 g, 71.9% yield) as brown oil.

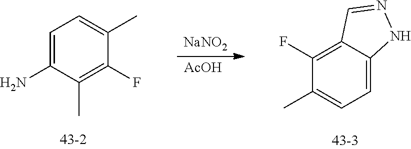

To a solution of 43-2 (4.00 g, 24.8 mmol) in acetic acid (20 mL) was added sodium nitrite (3.42 g, 49.4 mmol) at 20° C. The mixture was stirred for 2 hours at 20° C. The mixture was adjusted to pH-8 with an aqueous solution of sodium hydroxide (6 M) drop wise. The aqueous phase was extracted with ethyl acetate (300 mL*4), dried over anhydrous sodium sulfate, filtered, and the filtrate was concentrated under reduced pressure. The residue was purified by column chromatography on silica gel (petroleum ether:ethyl acetate=20:1, 10:1) and reversed-phase HPLC (0.1% trifluoroacetic acid/MeCN condition) to afford 4-fluoro-5-methyl-1H-indazole (43-3) (0.08 g, 1.8% yield) as an off-white solid.

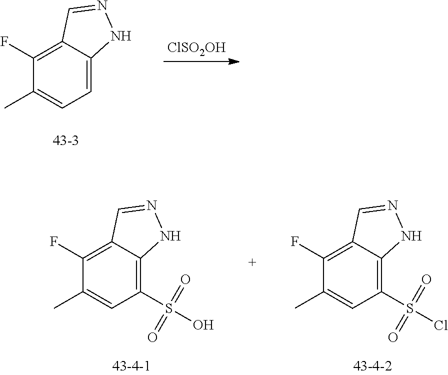

A round bottom flask was charged with chlorosulfonic acid (0.3 mL) and cooled to 0° C. To this was added 43-3 (0.06 g, 0.33 mmol) slowly at 0° C. and this mixture was stirred at 0° C. for 0.5 hour under nitrogen atmosphere. The mixture was added into ice water (15 mL) drop wise and extracted with ethyl acetate (30 mL*3). The combined organic phase was washed with brine (30 mL), dried over anhydrous sodium sulfate, filtered and the filtrate was concentrated under reduced pressure to afford 4-fluoro-5-methyl-1H-indazole-7-sulfonyl chloride (43-3-2) (0.015 g, 12.2% yield) as a brown solid. The aqueous phase was purified by reversed-phase HPLC (0.1% trifluoroacetic acid/MeCN condition) to afford 4-fluoro-5-methyl-1H-indazole-7-sulfonic acid (43-4-1) (0.03 g, 39.0% yield) as a white solid.

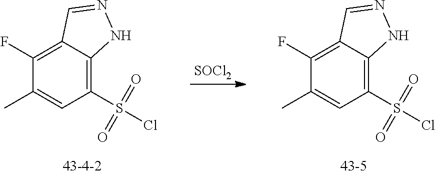

A round bottom flask was charged with 43-4-2 (0.03 g, 0.13 mmol). To this was added thionyl chloride (2 mL, 27.6 mmol) and dimethyl formamide (0.002 g, 0.027 mmol) at 20° C. The mixture was heated to 80° C. and stirred for 1 hour. The mixture was concentrated under reduced pressure to afford 43-5 (0.03 g, crude) as a yellow solid and used directly without further purification.

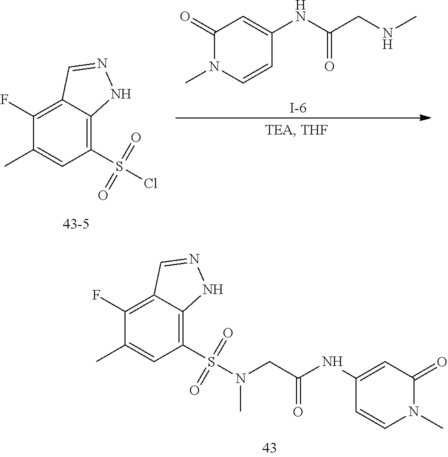

To a mixture of I-6 (0.028 g, 0.12 mmol) and triethylamine (0.084 mL, 0.60 mmol) in tetrahydrofuran (1 mL) was added 43-5 (0.03 g, 0.12 mmol) in tetrahydrofuran (1 mL) drop wise at 0° C. The reaction was warmed to 20° C. and stirred for 1 hour. The mixture was poured into water (10 mL) and extracted with ethyl acetate (30 mL*3). The combined organic phase was washed with brine (20 mL), dried over anhydrous sodium sulfate, filtered, and the filtrate was concentrated under reduced pressure. The crude product was purified by prep-HPLC (column: Waters Xbridge 150*25 mm*5 um; mobile phase: [Water-ACN]; B %: 15%-45%, 9 min) followed by prep-HPLC (column: Waters Xbridge 150*25 mm*5 um; mobile phase: [water (0.05% ammonia hydroxide v/v)-ACN]; B %: 15%-45%, 10 min) to afford 2-(4-fluoro-N,5-dimethyl-1H-indazole-7-sulfonamido)-N-(1-methyl-2-oxo-1,2-dihydropyridin-4-yl)acetamide (43) (0.007 g, 12% yield) as a white solid.

LCMS of 43: m/z 408.0 [M+H]⁺

¹H-NMR of 43: (Methanol-d₄, 400 MHz): δ 8.19 (s, 1H), 7.77 (d, J=6.4 Hz, 1H), 7.55 (d, J=8.0 Hz, 1H), 6.81 (d, J=2.4 Hz, 1H), 6.58 (dd, J=7.2, 2.4 Hz, 1H), 4.15 (s, 2H), 3.50 (s, 3H), 2.91 (s, 3H), 2.40 (d, J=2.0 Hz, 3H)

### Synthesis of 2-((N,5-dimethyl-3-((methylamino)methyl)-1H-indazole)-7-sulfonamido)-N-(1-methyl-2-oxo-1,2-dihydropyridin-4-yl)acetamide (Example 44)

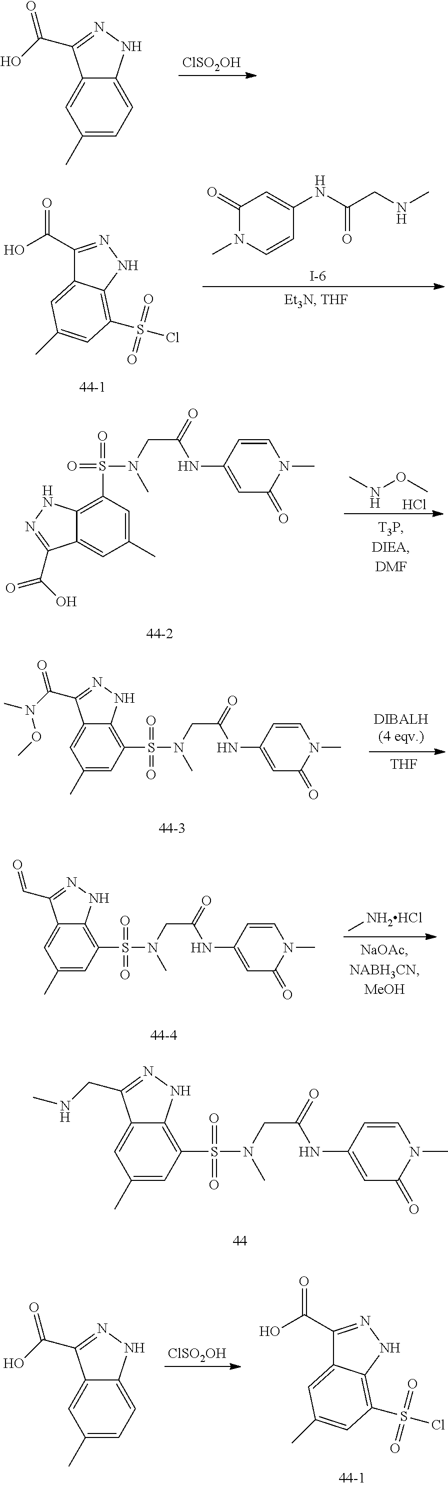

A round bottom flask was charged with sulfurochloridic acid (17.5 g, 150 mmol) and cooled to 0° C. To this was added 5-methyl-1H-indazole-3-carboxylic acid (1.00 g, 5.68 mmol) slowly at 0° C. The mixture was stirred at 20° C. for 30 minutes, then heated to 50° C. and stirred for 6 hours under nitrogen atmosphere. The mixture was poured into ice-water (120 mL) and stirred for 10 minutes—causing a solid to precipitate out. The formed solid was collected by filtration to afford 7-(chlorosulfonyl)-5-methyl-1H-indazole-3-carboxylic acid (44-1) (0.95 g, 3.01 mmol, 53% yield) as a yellow solid.

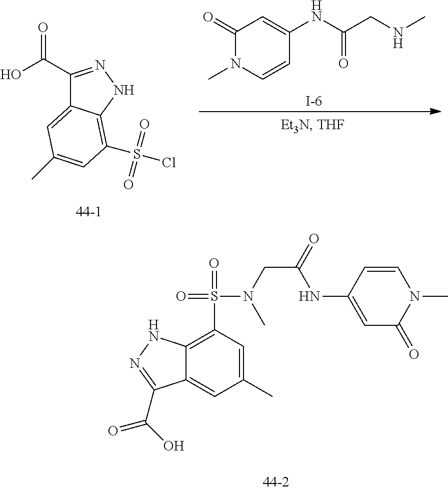

To a solution of I-6 as the hydrochloric salt (0.17 g, 0.73 mmol) in dimethyl formamide (2 mL) was added triethylamine (0.29 g, 2.91 mmol) and 44-1 (0.20 g, 0.728 mmol) at 0° C. The mixture was stirred at 0° C. for 1 hour. The residue was purified by reversed-phase HPLC (0.1% TFA condition) to afford 5-methyl-7-(N-methyl-N-(2-((1-methyl-2-oxo-1, 2-dihydropyridin-4-yl)amino)-2-oxoethyl)sulfamoyl)-1H-indazole-3-carboxylic acid (44-2) (0.11 g, 0.248 mmol, 34% yield) as a yellow solid.

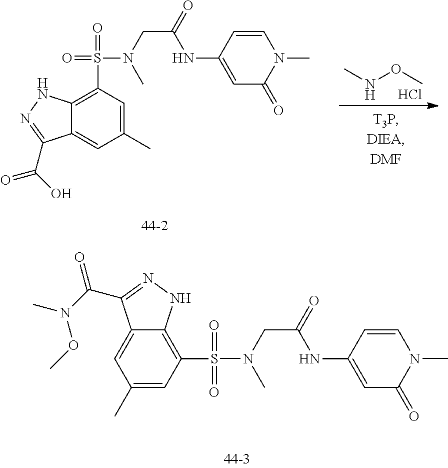

To a solution of 44-2 (0.12 g, 0.28 mmol) in dimethyl formamide (2 mL) was added N-methoxymethanamine as the hydrochloric salt (0.04 g, 0.42 mmol) and diisopropylethylamine (0.19 mL, 1.11 mmol), then T₃P (0.25 mL, 0.42 mmol, 50% w/w in ethyl acetate) was added drop wise at 15° C. and the reaction mixture was stirred at 15° C. for 1 hour under a nitrogen atmosphere. The reaction was quenched with ice water (30 mL) and extracted with ethyl acetate (20 mL*3). The organic layers were washed with brine (10 mL*2), dried over anhydrous sodium sulfate, filtrated, and the filtrate was concentrated under reduced pressure to afford N-methoxy-N,5-dimethyl-7-(N-methyl-N-(2-((1-methyl-2-oxo-1,2-dihydropyridin-4-yl)amino)-2-oxoethyl)sulfamoyl)-1H-indazole-3-carboxamide (44-3) (0.12 g, 0.25 mmol, 79.1% yield) as a yellow solid, which was used directly without further purification.

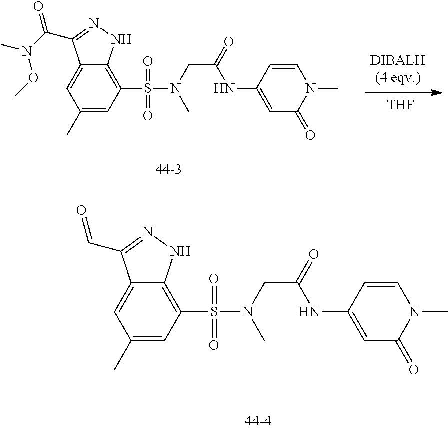

To a solution of 44-3 (0.08 g, 0.17 mmol) in tetrahydrofuran (2 mL) was added DIBAL-H (1 M, 1.5 mL) at −30° C. drop wise and the reaction mixture was stirred at −30° C. for 4 hours under nitrogen atmosphere. The reaction was quenched with a saturated aqueous solution of ammonium chloride (30 mL) at 0° C. and extracted with ethyl acetate (100 mL*3). The organic layer was washed with brine (20 mL*2), dried over anhydrous sodium sulfate, filtrated, and the filtrate was concentrated under reduced pressure to afford 2-(3-formyl-N,5-dimethyl-1H-indazole-7-sulfonamido)-N-(1-methyl-2-oxo-1,2-dihydropyridin-4-yl)acetamide (44-4) (0.08 g, 0.09 mmol, 57.1% yield) as a yellow solid, which was used directly without further purification.

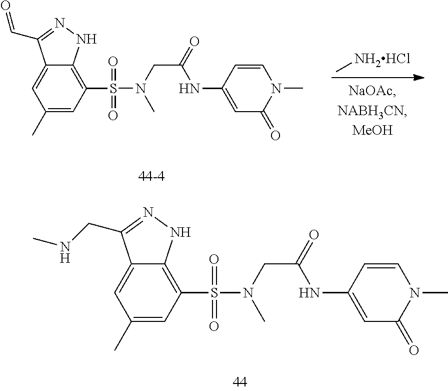

To a solution of 44-4 (0.06 g, 0.07 mmol) in methanol (4 mL) and dichloromethane (2 mL) was added methanamine hydrochloride (0.03 g, 0.43 mmol and sodium acetate (0.04 g, 0.43 mmol). After stirring for 10 minutes at 15° C., NaBH₃CN (0.02 g, 0.29 mmol) was added and the reaction mixture was stirred at 15° C. for 2 hours. The reaction was quenched with ice water (0.2 mL), filtrated, and the filtrate was concentrated under reduced pressure. The residue was purified by prep-HPLC (column: 3_Phenomenex Luna C₁₈ 75*30 mm*3 um; mobile phase: [water (0.05% HCl)-ACN]; B %: 8%-28%, 7 min) to afford 2-(N,5-dimethyl-3-((methylamino)methyl)-1H-indazole-7-sulfonamido)-N-(1-methyl-2-oxo-1,2-dihydropyridin-4-yl)acetamide (44) (0.013 g, 0.030 mmol, 32.4% yield) as a yellow solid.

LCMS for 44: m/z 433.7 [M+H]⁺

¹H-NMR for 44: (DMSO-d₆, 400 MHz): δ 13.48 (br. s, 1H), 10.52 (br. s, 1H), 9.31 (br. s, 2H), 8.11 (s, 1H), 7.69 (s, 1H), 7.62 (d, J=7.6 Hz, 1H), 6.66 (s, 1H), 6.36-6.35 (m, 1H), 4.55-4.52 (m, 2H), 4.20 (s, 2H), 3.68 (s, 3H), 2.85 (s, 3H), 2.62 (s, 3H), 2.49 (s, 3H).

### Synthesis of 2-((N,5-dimethyl-1H-indazole)-7-sulfonamido)-N-(imidazo[1,5-a]pyridin-7-yl)acetamide (Example 46)

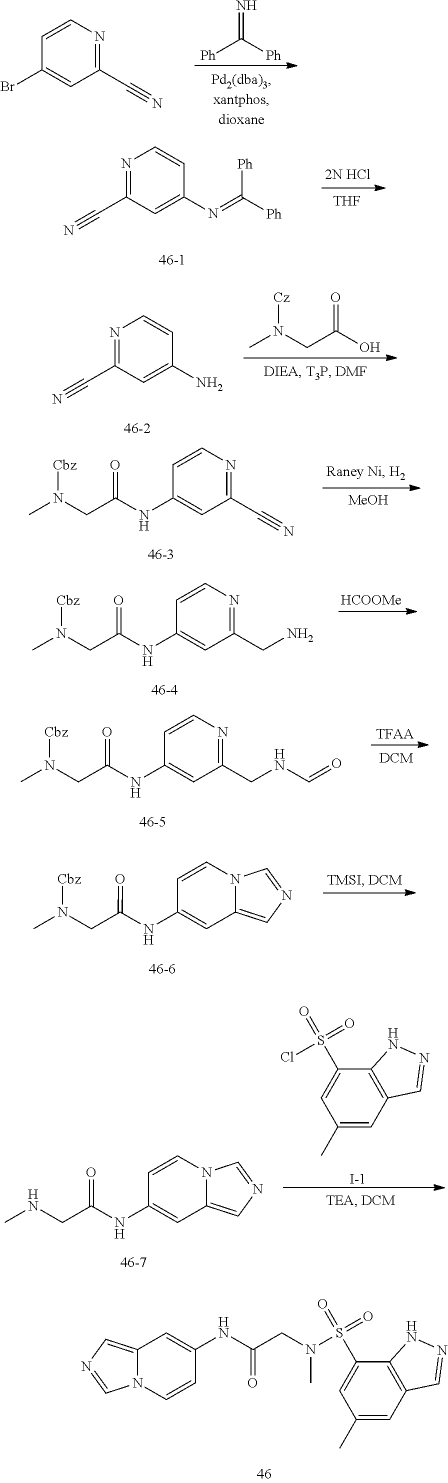

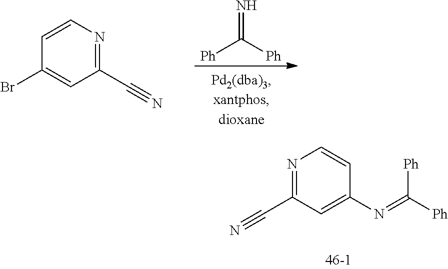 [SMILES: N#Cc1cc(Br)ccn1 ; N=C(c1ccccc1)c1ccccc1 ; N#Cc1cc(N=C(c2ccccc2)c2ccccc2)ccn1]

To a solution of 4-bromopicolinonitrile (5.00 g, 27.3 mmol), diphenylmethanimine (7.43 g, 41.0 mmol), Xantphos (3.16 g, 5.46 mmol) and cesium carbonate (17.8 g, 54.6 mmol) in dioxane (100 mL) was added Pd₂(dba)₃ (2.50 g, 2.73 mmol) under nitrogen. The mixture was stirred at 80° C. for 2.5 hours under a nitrogen atmosphere then diluted with ethyl acetate (100 mL), filtered, and the filtrate was concentrated under reduced pressure. The residue was purified by column chromatography (SiO₂, petroleum ether/ethyl acetate=200/1˜20/1) to afford 4-((diphenylmethylene)amino)picolinonitrile (46-1) (5.00 g, 14.8 mmol, 24.7% yield) as a yellow solid.

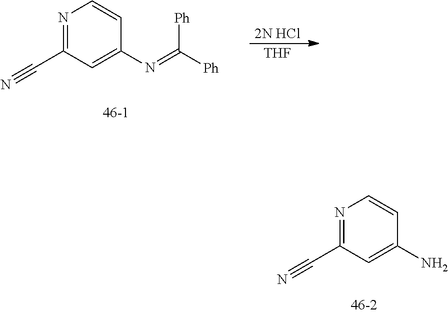 [SMILES: N#Cc1cc(N=C(c2ccccc2)c2ccccc2)ccn1 ; "2N HCl" ; N#Cc1cc(N)ccn1 ; "THF"]

To a solution of 46-1 (5.00 g, 14.8 mmol) in tetrahydrofuran (50 mL) was added hydrochloric acid (2 M, 50 mL). Then the mixture was stirred at 25° C. for 3 hours then washed with petroleum ether (50 mL*3). Then the pH of aqueous phase was adjusted to 10 with sodium carbonate (s). The aqueous phase was extracted with dichloromethane (50 mL*4). The combined organic phase was dried over anhydrous sodium sulfate, filtered, and the filtrate was concentrated under reduced pressure to afford 4-aminopicolinonitrile (46-2) (1.66 g, 13.9 mmol, 93.9% yield) as a yellow solid.

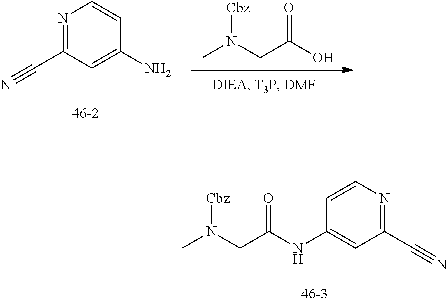 [SMILES: N#Cc1cc(N)ccn1 ; CN(CC(=O)O)C(=O)OCc1ccccc1 ; CN(CC(=O)Nc1ccnc(C#N)c1)C(=O)OCc1ccccc1]

To a solution of 46-2 (0.660 g, 5.54 mmol) and 2-(((benzyloxy) carbonyl)(methyl)amino)acetic acid (1.86 g, 8.32 mmol) in N, N-dimethylformamide (2 mL) was added N, N-diisopropylethylamine (3.8 mL, 21.8 mmol) and T₃P (4.9 mL, 8.24 mmol, 50% w/w in ethyl acetate) drop wise. The mixture was stirred at 50° C. for 2 hours then diluted with water (30 mL) and extracted with ethyl acetate (30 mL*6). The combined organic phase was dried over anhydrous sodium sulfate, filtered, and the filtrate was concentrated under reduced pressure. The residue was purified by column chromatography (SiO₂, petroleum ether/ethyl acetate=1/1) to afford benzyl (2-((2-cyanopyridin-4-yl)amino)-2-oxoethyl)(methyl) carbamate (46-3) (1.1 g, 3.39 mmol, 61.2% yield) as red oil.

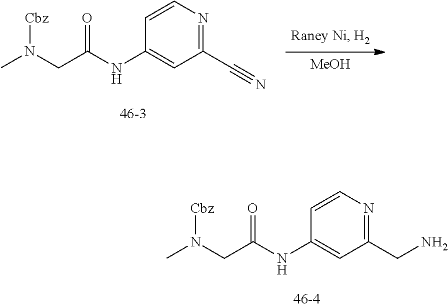 [SMILES: "MeOH" ; CN(CC(=O)Nc1ccnc(C#N)c1)C(=O)OCc1ccccc1 ; CN(CC(=O)Nc1ccnc(CN)c1)C(=O)OCc1ccccc1]

To a solution of 46-3 (1.10 g, 3.39 mmol) in methanol (1 mL) was added Raney-Ni (0.110 g) under nitrogen atmosphere. The suspension was degassed and purged with hydrogen for three times and stirred at 30° C. for 12 hours under hydrogen atmosphere (50 psi). The mixture was filtrated and the filter cake was rinsed with methanol (20 mL). The filtrate was concentrated under reduced pressure to afford benzyl (2-((2-(aminomethyl)pyridin-4-yl)amino)-2-oxoethyl)(methyl)carbamate (46-4) (1.10 g, 3.18 mmol, 93.9% yield) as purple oil.

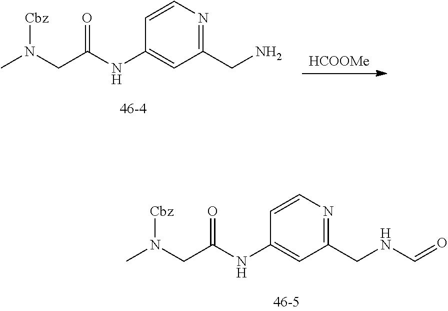

A mixture of 46-4 (1.10 g, 3.18 mmol) in methyl formate (20 mL, 330 mmol) was stirred at 80° C. for 36 hours. The mixture was concentrated under reduced pressure. The crude product was purified by reversed-phase HPLC (0.1% NH₃H₂O/MeOH condition) to afford benzyl (2-((2-(formamidomethyl)pyridin-4-yl)amino)-2-oxoethyl)(methyl)carbamate (46-5) (0.51 g, 1.38 mmol, 43.2% yield) as a white solid.

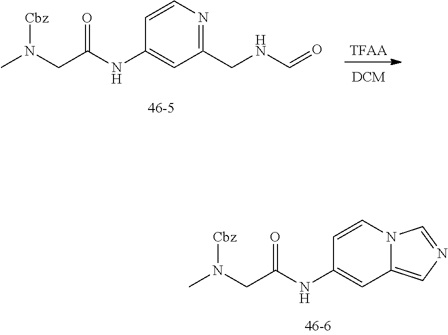 [SMILES: CN(CC(=O)Nc1ccnc(CNC=O)c1)C(=O)OCc1ccccc1 ; "TFAA" ; CN(CC(=O)Nc1ccn2cncc2c1)C(=O)OCc1ccccc1]

To a solution of 46-6 (0.310 g, 0.870 mmol) in dichloromethane (4 mL) at 0° C. was added trifluoroacetic anhydride (2 mL, 14.4 mmol) drop wise. Then the mixture was stirred at 0-25° C. for 12 hours under nitrogen atmosphere. The mixture was concentrated under reduced pressure. The residue was purified by prep-HPLC (column: Phenomenex Gemini-NX C₁₈ 75*30 mm*3 um; mobile phase: [water (0.1% TFA)-ACN]; B %: 20%-30%, 7 min) to afford benzyl (2-(imidazo[1,5-a]pyridin-7-ylamino)-2-oxoethyl)(methyl) carbamate (46-6) (0.04 g, 0.11 mmol, 12.9% yield) as colourless oil.

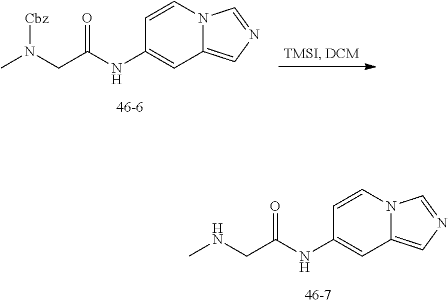 [SMILES: CN(CC(=O)Nc1ccn2cncc2c1)C(=O)OCc1ccccc1 ; CNCC(=O)Nc1ccn2cncc2c1]

To a solution of 46-6 (0.035 g, 0.103 mmol) in dichloromethane (1 mL) at 0° C. was added trimethylsilyl iodide (0.03 mL, 0.220 mmol). Then the mixture was stirred at 0-25° C. for 30 min under nitrogen. The mixture was quenched by methanol (5 mL) and the mixture was concentrated under reduced pressure. The crude product was purified by reversed-phase HPLC (0.1% NH₃H₂O/MeCN condition) to afford N-(imidazo[1,5-a]pyridin-7-yl)-2-(methylamino)acetamide (46-7) (0.01 g, 0.047 mmol, 45.7% yield) as a brown solid.

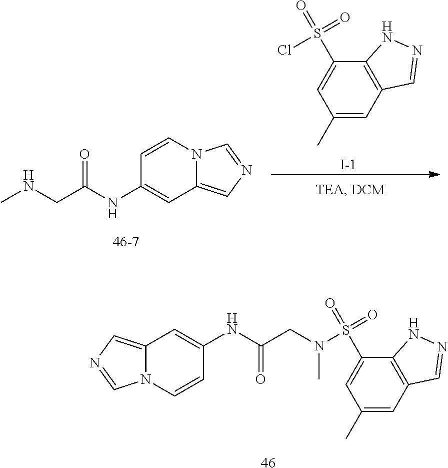 [SMILES: CNCC(=O)Nc1ccn2cncc2c1 ; Cc1cc(S(=O)(=O)Cl)c2[nH]ncc2c1 ; [I-] ; Cc1cc(S(=O)(=O)N(C)CC(=O)Nc2ccn3cncc3c2)c2[nH]ncc2c1 ; "46"]

To a solution of 46-7 (0.01 g, 0.04 mmol) in dichloromethane (0.5 mL) at 0° C. was added triethylamine (0.02 mL, 0.14 mmol) and I-1 (0.01 g, 0.04 mmol). Then the mixture was stirred at 0-25° C. for 1 hour. The mixture was concentrated under reduced pressure. The residue was purified by prep-HPLC (column: Phenomenex Gemini NX-Cis (75*30 mm*3 um); mobile phase: [water (10 mM NH₄HCO₃)-ACN]; B %: 15%-45%, 8 min) to afford 2-(N,5-dimethyl-1H-indazole-7-sulfonamido)-N-(imidazo[1,5-a]pyridin-7-yl)acetamide (46) (0.011 g, 0.03 mmol, 68.3% yield) as a brown solid.

LCMS of 46: m/z 399.1 [M+H]⁺

¹H-NMR of 46: (CD₃OD, 400 MHz): δ 8.22 (s, 1H), 8.18 (d, J=7.2 Hz, 1H), 8.13 (s, 1H), 7.92-7.88 (m, 2H), 7.75 (s, 1H), 7.25 (s, 1H), 6.72 (dd, J=7.2, 2.0 Hz, 1H), 4.14 (s, 2H), 2.91 (s, 3H), 2.52 (s, 3H)

### Synthesis of (S)—N-cyclohexyl-1-((5-methyl-1H-indazol-7-yl)sulfonyl)azetidine-2-carboxamide (Example 70)

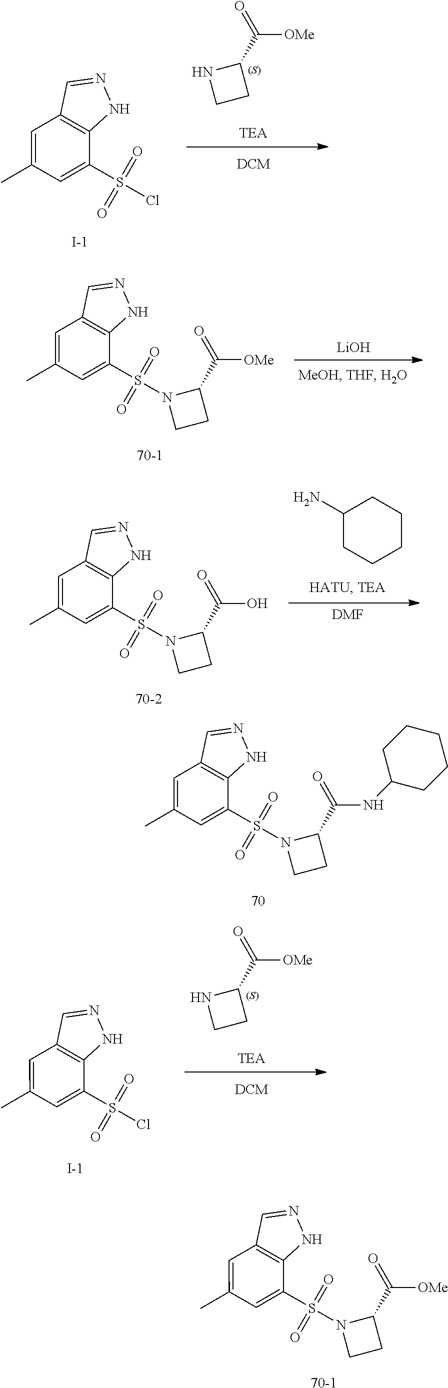

To a solution of (S)-methyl azetidine-2-carboxylate (0.800 g, 5.28 mmol) in DCM (10 mL) was added TEA (2.94 mL, 21.1 mmol) and I-1 (1.58 g, 6.86 mmol) at 0° C. The mixture was stirred at 18° C. for 12 hr then diluted with water (20 mL) and extracted with EtOAc (20 mL*3). The combined organic layers were washed with brine (50 mL), dried over Na₂SO₄, filtered, and concentrated under reduced pressure. The residue was purified by column chromatography (SiO₂, Petroleum ether/Ethyl acetate=5/1 to 1/1) to give (S)-methyl 1-((5-methyl-1H-indazol-7-yl)sulfonyl)azetidine-2-carboxylate (70-1) (1.00 g, 60.0% yield) as a yellow solid.

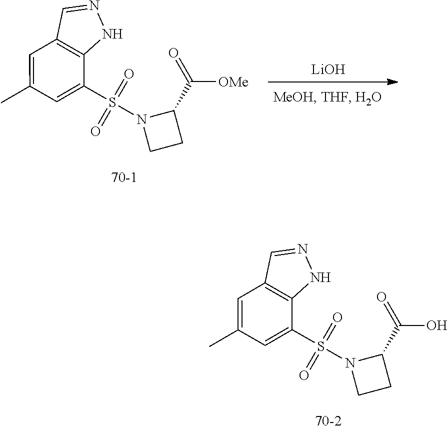

To a solution of 70-1 (0.900 g, 2.91 mmol) in a mixture of MeOH (8.1 mL), THF (2.7 mL) and water (2.7 mL) was added LiOH·H₂O (0.244 g, 5.82 mmol). The mixture was stirred at 40° C. for 0.5 hr. The reaction mixture was adjusted to pH=1 with 1N HCl, filtered, and the filter cake was concentrated under reduced pressure to give (S)-1-((5-methyl-1H-indazol-7-yl)sulfonyl)azetidine-2-carboxylic acid (70-2) (0.680 g, 75.9% yield) as a yellow solid.

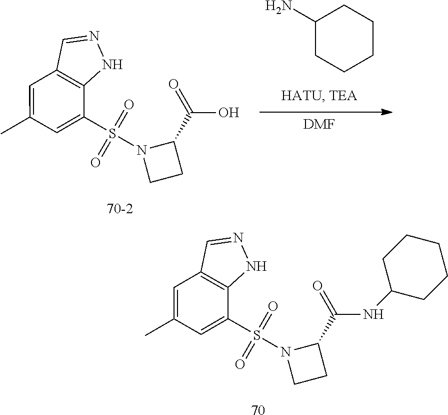

To a solution of 70-2 (0.0776 mg, 0.263 mmol) in DMF (1 mL) was added HATU (0.0999 g, 0.263 mmol) and TEA (0.0664 g, 0.657 mmol). The mixture was stirred at 18° C. for 0.5 hr. Then cyclohexylamine (0.219 mmol) was added. The mixture was stirred at 18° C. for 11.5 hr. The reaction mixture was diluted with aq. NaHCO₃ (5 mL) and extracted with EtOAc (8 mL*3). The combined organic layers were washed with brine (15 mL), dried over Na₂SO₄, filtered, and concentrated under reduced pressure. The residue was purified by prep-HPLC (neutral condition) then further purified by Chiral SFC (Instrument: Waters SFC150AP preparative SFC; Column: Chiralpak AD, 250*30 mm i.d. 10 um; Mobile phase: A for CO₂ and B for IPA (0.1% NH₃H₂O); Gradient: B %=50% isocratic elution mode; Flow rate: 70 g/min; Column temperature: 35° C.; System back pressure: 150 bar), with the major peak assigned as the (S)-enantiomer, to provide (S)—N-cyclohexyl-1-((5-methyl-1H-indazol-7-yl)sulfonyl)azetidine-2-carboxamide (70) (0.0330 g, 36.9% yield) as a white solid.

LCMS for 70: m/z 377.1 [M+H]⁺

¹H-NMR for 70: (400 MHz, METHANOL-d4) δ 8.17 (s, 1H), 7.97 (s, 1H), 7.76 (s, 1H), 4.65 (t, J=8.6 Hz, 1H), 3.87 (q, J-=8.6 Hz, 1H), 3.74-3.60 (z, 2H)-2.56 (s, 3H), 2.36-2.22 (m, 2H), 1.94-1.72 (m, 4H), 1.71-1.60 (m, 1H), 1.47-1.31 (m, 2H), 1.29-1.14 (m, 3H)

The following compounds were synthesized in a similar manner as shown from 70-2 to 70 using the amide coupling conditions outlined in the General Experimental Method B section above.

| | | Coupling | LCMS | |
|---|---|---|---|---|
| Example | Reactant | Conditions | m/z | ¹H-NMR |
| 71 | 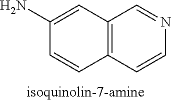 [SMILES: Nc1ccc2ccncc2c1] | 2 | 422.2 | (400 MHz, METHANOL-d4) δ 9.17 (s, 1H), 8.50 (d, J = 2.0 Hz, 1H), 8.37 (d, J = 5.7 Hz, 1H)-8.14 (s, 1H), 7.95-7.90 (m, 2H), 7.87-7.83 (m, 1H), 7.79-7.76 (m, 2H), 4.88 (t, J-= 8.7 Hz, 1H), 3.97-3.90 (m, |
| | | | | 1H), 3.71 (dt, J = 4.8, 7.8 Hz, |
| | | | | 1H)-2.51 (s, 3H), 2.48-2.39 |
| | | | | (m, 2H) |
| 74 | 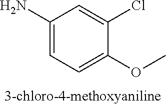 | 2 | 435.1 | 35 (400 MHz, METHANOL-d4) δ 8.18 (s, 1H), 7.96 (s, 1H), 7.80 (s, 1H), 7.70 (d, J = 2.5 Hz, 1H), 7.45 (dd, J = 2.5, 8.9 Hz, 1H), 7.07 (d, J = 9.0 Hz, 1H), 4.82 (t, J = 8.6 Hz, 1H), 3.96-3.88 (m, 4H), 3.75-3.67 |
| | | | | (m, 1H)-2.55 (s, 3H), 2.46- |
| | | | | 2.35 (m, 2H) |
| 80 | 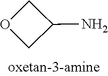 | 1 | 351.1 | (400 MHz, DMSO-d6) δ = 13.69 (br s, 1H), 8.97 (br s, 1H), 8.20 (s, 1H), 7.94 (s, 1H)-7.65 (s, 1H), 4.91-4.78 (m, 1H), 4.77-4.64 (m, 4H), 4.51-4.33 |
| | | | | (m, 3H), 3.76 (q, J = 8.5 Hz, |
| | | | | 1H), 3.52 (dt, J = 4.-, 8.1 Hz, |
| | | | | 1H), 2.38-2.08 (m, 3H) |
| 81 | 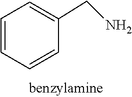 | 1 | 385.1 | (400 MHz, DMSO-d6) δ −13.79 (s, 1H), 8.87-8.66 (m, 1H) −8.19 (s, 1H), 8.01-7.84 (m, 1H)- 7.65 (s, 1H), 7.40-7.14 (m, 5H), 4.76 (t, J- = 8.4 Hz, 1H), 4.48-4.26 (m, 2H), 3.86- |
| | | | | 3.64 (m, 1H), 3-51 (br s, 1H), |
| | | | | 2.45-2.43 (m, 3H), 2.33-2.05 |
| | | | | (m, 2H) |
| 82 | 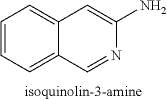 [SMILES: Nc1cc2ccccc2cn1] | 2 | 422.1 | (400 MHz, DMSO-d6) δ 13.51 (br s, 1H), 10.72 (br s, 1H), 9.17 (s, 1H), 8.49 (s, 1H), 8.22 (s, 1H), 8.07 (d, J = 8.3 Hz, 1H), 7.95 (d, J- = 4.4 Hz, 2H), 7.78- 7.66 (m, 2H), 7.60-7.43 (m, 1H), 5.05-4.91 (m, 1H), 3.68 |
| | | | | (br s, 2H), 3.47 (s, 1H) −2.63 (s, |
| | | | | 1H), 2.39 208ydroxyamm, 4H) |
| 84 | 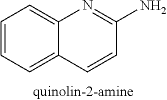 [SMILES: Nc1ccc2ccccc2n1] | 2 | 422.1 | (400 MHz, METHANOL-d4) δ 8.36 (s, 2H), 8.18 (s, 1H) −7.96 (s, 1H), 7.94-7.87 (m, 2H), 7.84 (s, 1H), 7.75 (dt, J = 1.-, 7.7 Hz, 1H), 7.60-7.48 (m, 1H), 3.94 (q, J = 8.6 Hz, 1H), 3.78 (br d, J = 5.7 Hz, 1H) |
| | | | | −2.56 (s, 3H), 2.49-2.41 (m, |
| | | | | 2H), 2.05 (s, 1H) |
| 85 | 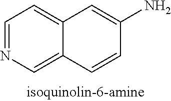 [SMILES: Nc1ccc2cnccc2c1] | 2 | 422.1 | (400 MHz, DMSO-d6) δ 13.46 (s, 1H), 10.45 (s, 1H), 9.19 (s, 1H), 8.42 (d, J = 5.7 Hz, 1H), 8.32 (br s, 1H), 8.21 (s, 1H), 8.09 (d, J = 9.0 Hz, 1H), 7.93 (s, 1H), 7.76 (d, J = 5.7 Hz, 2H), 7.68 (br s, 1H), 4.85 (t, |
| | | | | J = 8.1 Hz, 1H), 3.84-3.72 (m, |
| | | | | 1H), 3.71-3.58 (m, 1H) −3.25 |
| | | | | (s, 1H), 2.45-2.43 (m, 3H), |
| | | | | 2.33 (br s, 1H) |
| 86 | 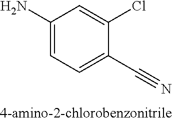 | 4 | 429.9 | (400 MHz, METHANOL-d4) δ 8.18 (s, 1H), 8.05 (d, J = 1.9 Hz, 1H) −7.96 (s, 1H), 7.82- 7.75 (m, 2H), 7.67 (dd, J = 2.-, 8.6 Hz, 1H), 4.84-4.82 (m, 1H), 3.94 (q, J = 8.5 Hz, 1H), 3.73 (dt, J = 4.9, 7.8 Hz, 1H) −2.55 (s, 3H), 2.50 |
| | | | | 209ydroxyamm, 2H) |
| 87 | 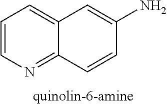 [SMILES: Nc1ccc2ncccc2c1] | 3 | 422.1 | (400 MHz, METHANOL-d4) δ 8.81 (dd, J = 1.5, 4.2 Hz, 1H), 8.46 (d, J = 2.3 Hz, 1H), 8.37 (d, J = 8.2 Hz, 1H), 8.19 (s, 1H), 8.03 (d, J = 9.2 Hz, 1H) −7.96 (s, 1H), 7.91-7.77 (m, 2H), 7.57 (dd, J = 4.2, 8.4 Hz, |
| | | | | 1H), 4-95 (br s, 1H), 4.05- |
| | | | | 3.90 (m, 1H), 3.84-3.69 (m, |
| | | | | 1H) −2.55 (s, 3H), 2.53-2.40 |
| | | | | (m, 2H) |
| 106 | 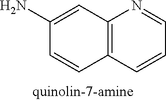 [SMILES: Nc1ccc2cccnc2c1] | 3 | 422.1 | (400 MHz, METHANOL-d4) δ 8.84 (dd, J = 1.7, 4.3 Hz, 1H), 8.46 (s, 1H), 8.35 (d, J = 7.8 Hz, 1H) −8.17 (s, 1H), 8.02- 7.91 (m, 2H), 7.90-7.76 (m, 2H), 7.50 (dd, J = 4.-, 8.3 Hz, 1H), 4.98-4.90 (m, 1H), 4.05- |
| | | | | 3.87 (m, 1H), 3.83-3.71 (m, |
| | | | | 1H) −2.55 (s, 3H), 2.51-2.39 |
| | | | | (m, 2H) |
| 128 | 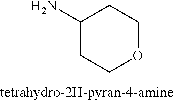 | 1 | 379.1 | (400 MHz, DMSO-d6) δ 13.98 (br s, 1H), 8.24 (s, 2H), 7.97 (s, 1H), 7.68 (s, 1H), 4.80 (t, J = 8.5 Hz, 1H), 3.95-3.76 (m, 4H), 3.56-3.38 (m, 3H), 2-50 (br s, 3H), 2.37-2.31 (m, 1H), 2.25-2.16 (m, 1H), 1.73 (br dd, |
| | | | | J = 1.6-12.8 Hz, 2H), 1.53- |
| | | | | 1.33 (m, 2H) |
| 129 | 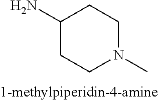 | 1 | 392.1 | (400 MHz, METHANOL-d4) δ 8.18 (s, 1H), 7.97 (s, 1H), 7.76 (s, 1H), 4.68 (t, J = 8.6 Hz, 1H), 3.87 (q, J = 8.6 Hz, 1H), 3.80- 3.71 (m, 1H), 3.69-3.60 (m, 1H), 2.87 (br d, J = 10.4 Hz, 2H) −2.56 (s, 3H), 2.36-2.17 |
| | | | | (m, 7H), 1.96-1.82 (m, 2H), |
| | | | | 1.65-1.50 (m, 2H) |
| 131 | 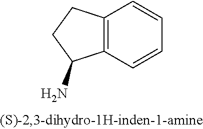 | 2 | 411.1 | (400 MHz, METHANOL-d4) δ 8.11 (s, 1H), 7.90 (s, 1H) −7.73 (s, 1H), 7.26-7.13 (m, 4H), 5.40 (t, J = 7.7 Hz, 1H), 4.71 (t, J = 8.2 Hz, 1H), 3.84 (q, J = 8.1 Hz, 1H), 3.65 (q, J = 7.0 Hz, 1H), 3.04-2.96 (m, 1H), 2.90- 2.84 (m, 1H), 2.57-2.46 (m, |
| | | | | 4H), 2.35-2.27 (m, 2H), 1.90- |
| | | | | 1.79 (m, 1H) |
| 135-R | 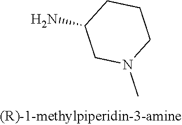 | 1 | 392.1 | (400 MHz, METHANOL-d4) δ 8.18 (s, 1H), 7.98 (s, 1H), 7.76 (s, 1H), 4.62 (t, J = 8.6 Hz, 1H), 4.03-3.78 (m, 2H), 3.75- 3.62 (m, 1H), 2.89-2.72 (m, 1H), 2.57 (s, 4H) −2.30 (s, 4H), 2.24-2.15 (m, 1H), 2.14-1.99 (m, 1H), 1.77 (br d, J = 9.3 Hz, |
| | | | | 2H), 1.69-1.55 (m, 1H), 1.38- |
| | | | | 1.25 (m, 1H), 1.17 (d, J = 6.1 |
| | | | | Hz, 1H) |
| 135-S | 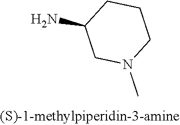 | 1 | 392.1 | (400 MHz, METHANOL-d4) δ 1.29-1.41 (m, 1 H) 1-66 (br s, 1 H) 1.74-1.87 (m, 2 H) 1.93- 2.04 (m, 1 H) 2.23-2.37 (m, 6 H −2.57 (s, 4 H) 2.68-2.80 (m, 1 H) 3.67 (td, J = 8.00-4.50 Hz, 1 H) 3.83-3.89 (m, 1 H) 3.90- 3.98 (m, 1 H) 4.63 (t, J = 8.63 |
| | | | | Hz, 1 H) 7.77 (d, J = 0.88 Hz, 1 |
| | | | | H) 7.98 (s, 1 H) 8.18 (s, 1 H) |
| 138 | 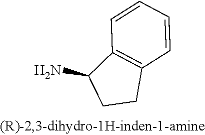 | 2 | 411.1 | (400 MHz, METHANOL-d4) δ 8.16 (s, 1H), 7.94 (s, 1H) −7.77 (s, 1H), 7.30-7.21 (m, 4H), 5.44 (t, J = 7.6 Hz, 1H), 4.75 (t, J = 8.5 Hz, 1H), 3.94-3.85 (m, 1H), 3.71-3.66 (m, 1H), 3.07-3.00 (m, 1H), 2.94-2.86 (m, 1H) −2.55 (s, 3H), 2.39- 2.33 (m, 2H), 1.93-1.83 (m, |
| | | | | 1H), 1.21-1.16 (m, 1H) |
| 139 | 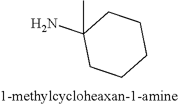 | 2 | 391.2 | (400 MHz, METHANOL-d4) δ 8.18 (s, 1H), 7.99 (s, 1H), 7.76 (s, 1H), 4.62 (t, J = 8.5 Hz, 1H), 3.87 (q, J = 8.5 Hz, 1H), 3.70 (td, J = 3.9, 8.2 Hz, 1H) −2.57 (s, 3H), 2.36-2.22 (m, 2H), 2.10 (br d, J = 13.1 Hz, 1H), |
| | | | | 1.97 (br d, J − 11.8 Hz, 1H), |
| | | | | 1.61-1.35 (m, 7H), 1.31 (s, |
| | | | | 3H), 1.17 (d, J = 6.2 Hz, 1H) |
| 143 | I-18 | 2 | 484.1 | (400 MHz, METHANOL-d₄) δ |
| | | | | 8.17 (s, 1H), 7.96 (s, 1H), 7.79 |
| | | | | (s, 1H), 7.09 (d, J = 2.5 Hz, |
| | | | | 1H), 6.96 (dd, J = 2.4, 8.7 Hz, |
| | | | | 1H), 6.26 (d, J = 8.6 Hz, 1H) |
| | | | | −4.91 (s, 2H), 4.81-4.75 (m, |
| | | | | 2H), 4.59-4.55 (m, 2H), 4.39- |
| | | | | 4.36 (m, 2H), 3.95-3.88 (m, |
| | | | | 1H), 3.73-3.67 (m, 1H), 3.22 |
| | | | | (br d, J = 4.5 Hz, 2H) −2.55 (s, |
| | | | | 3H), 2.42-2.36 (m, 2H) |
| 144 | I-15 | 2 | 512.1 | (400 MHz-METHANOL-d4) |
| | | | | δ 2.35-2.44 (m, 4 H) 2.55 (s, 5 |
| | | | | H) 3.55 (br t, J = 10.26 Hz, 4 H) |
| | | | | 3.70 (q, J = 6.50 Hz, 2 H) 3.93 |
| | | | | (q, J = 8.63 Hz, 2 H) 4.38 (br d, |
| | | | | J = 1.38 Hz, 4 H) 7.80 (s, 2 H) |
| | | | | 7.97 (s, 2 H) 8.18 (s, 2 H) |
| 147 | I-25 | 2 | 500.1 | (CD3OD, 400 MHz): δ 8.16 (s, |
| | | | | 1H), 7.94 (s, 1H), 7.78 (s, 1H), |
| | | | | 7.09 (d, J = 2.4 Hz, 1H), 7.01 |
| | | | | (d, J = 8.8 Hz, 1H), 6.92 (dd, |
| | | | | J = 8.-, 2.4 Hz, 1H), 4.81-4.77 |
| | | | | (m, 1H), 4.25 (t, J = 4.8 Hz, |
| | | | | 2H), 3.94-3.87 (m, 1H), 3.71- |
| | | | | 3.65 (m, 1H), 3.61 (s, 2H), 3.37 |
| | | | | (t, J = 4.8 Hz, 2H) −2.53 (s, |
| | | | | 3H), 2.42-2.33 (m, 2H), 1.29 |
| | | | | (s, 6H) |
| 200 | 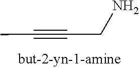 | 2 | 347.1 | (CDCl3, 400 MHz) δ 8.16 (s, 1H), 7.89 (s, 1H), 7.66 (s, 1H), 7.05 (br. s, 1H), 4.41 (t, J = 8.4 Hz, 1H), 4.18-4.01 (m, 2H), 3.83-3.75 (m, 1H), 3.72-3.63 |
| | | | | (m, 1H) −2.58 (s, 3H), 2.39- |
| | | | | 2.30 (m, 1H), 2.28-2.20 (m, |
| | | | | 1H), 1.87 (s, 3H) |
| 201 | 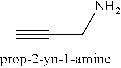 | 2 | 333.0 | (400 MHz, CDCl₃) δ = 8.16 (s, 1H), 7.90 (s, 1H), 7.66 (d, J = 0.8 Hz, 1H), 7.19 (s, 1H), 4.45- 4.40 (m, 1H), 4.19-4.17 (m, 1H), 4.14-4.12 (m, 1H), 3.80- |
| | | | | 3.77 (m, 1H), 3.67 (q, J = 4.8 |
| | | | | Hz, 1H), 2.58 (s, 3H), 2.41- |
| | | | | 2.39 (m, 1H), 2.29-2.20 (m, |
| | | | | 1H) |
| 202 | 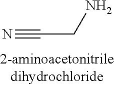 | 2 | 334.0 | (CDCl3, 400 MHz) δ 8.22 (s, 1H) −7.93 (s, 1H), 7.77-7.61 (m, 2H), 4.53-4.43 (m, 1H), 4.43-4.33 (m, 1H), 4.28-4.17 (m, 1H), 3.86-3.76 (m, 1H), 3.72-3.59 (m, 1H)-2.59 (s, |
| | | | | 3H), 2.47-2.33 (m, 1H), 2.31- |
| | | | | 2.19 (m, 1H) |
| 205 | 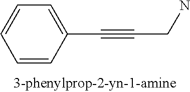 | 2 | 409.1 | (DMSO-d6, 400 MHz) δ 13.32 (br. s, 1H), 8.64 (t, J = 5.2 Hz, 1H), 8.19 (s, 1H), 7.91 (s, 1H), 7.64 (d, J = 1.2 Hz, 1H), 7.43- 7.40 (m, 2H), 7.39-7.36 (m, 3H), 4.14 (d, J = 5.2 Hz, 2H), |
| | | | | 3.90 (s, 2H), 2.74 (s, 3H), 2.48 |
| | | | | (s, 3H) |

### Synthesis of (R)-1-((5-methyl-1H-indazol-7-yl)sulfonyl)-N-(1-methyl-2-oxo-1,2-dihydropyridin-4-yl)piperidine-3-carboxamide (Example 96)

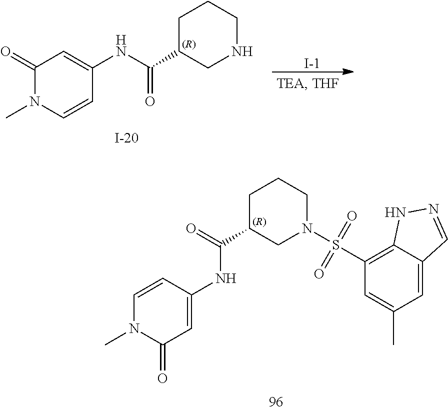

To a solution of I-20 as the hydrochloric salt (0.060 g, 0.221 mmol) in tetrahydrofuran (1 mL) at 0° C. was added triethylamine (0.15 mL, 1.08 mmol) and I-1 (0.051 g, 0.221 mmol). Then the mixture was stirred at 25° C. for 1 hour. The mixture was concentrated under reduced pressure at 35° C. The residue was purified by prep-HPLC (column: Waters Xbridge 150*25 mm*5 um; mobile phase: [water (10 mM NH₄HCO₃)-ACN]; B %: 18%-48%, 9 min) to afford (R)-1-((5-methyl-1H-indazol-7-yl)sulfonyl)-N-(1-methyl-2-oxo-1,2-dihydropyridin-4-yl)piperidine-3-carboxamide (96) (0.034 g, 0.076 mmol, 32.7% yield) as a white solid.

LCMS of 96: m/z 430.1 [M+H]⁺

¹H-NMR of 96: (CD₃OD, 400 MHz) δ 8.12 (s, 1H), 7.89 (s, 1H), 7.63 (s, 1H), 7.54 (d, J=7.2 Hz, 1H), 6.90 (d, J=0.8 Hz, 1H), 6.61 (dd, J=7.-, 0.8 Hz, 1H), 4.05-3.98 (m, 1H), 3.89-3.81 (m, 1H), 3.50 (s, 3H), 2.64 (s, 1H)-2.53 (s, 3H), 2.52-2.46 (m, 1H), 2.37-2.29 (m, 1H), 1.95-1.79 (m, 2H), 1.69-1.56 (m, 1H), 1.47-1.36 (m, 1H)

The following compound was synthesized in a similar manner to 96 using the reactant indicated in replace of I-20.

| | | LCMS | |
|---|---|---|---|
| Example | Reactant | m/z | ¹H-NMR |
| 97 | I-21 | 416.0 | (400 MHz, DMSO-d₆ + D₂O) δ 8.16 (s, |
| | | | 1H)- 7.89 (s, 1H), 7.58-7.56 (m, 2H), |
| | | | 6.76 (d, J = 2.0 Hz, 1H), 6.38 (dd, J = |
| | | | 4.-, 2.4 Hz, 1H), 3.68-3.51 (m, 1H)- |
| | | | 3.33 (s, 3H), 3.25-3.23 (m, 3H), 3.03- |
| | | | 2.98 (m, 1H)- 2.44 (s, 3H), 1.82-1.79 |
| | | | (m, 2H), 1.56-1.50 (m, 2H) |
| 213 | I-57 | 416.3 | (CDCl₃, 400 MHz) δ 9.53 (br. s, 1H), |
| | | | 8.06 (s, 1H), 7.76 (s, 1H), 7.63 (s, 1H), |
| | | | 7.23 (d, J = 7.6 Hz, 1H), 6.95 (d, J = |
| | | | 7.2 Hz, 1H)- 6.51 (s, 1H), 3.74-3.70 (m, |
| | | | 1H)- 3.50 (s, 3H), 3.46-3.32 (m, 3H), |
| | | | 3.15 (t, J = 7.6 Hz, 1H)- 2.52 (s, 3H), |
| | | | 2.14-2.13 (m, 2H) |

### Synthesis of 2-((5-ethyl-N-methyl-1H-indazole)-7-sulfonamido)-N-(1-methyl-2-oxo-1,2-dihydropyridin-4-yl)acetamide (Example 117)

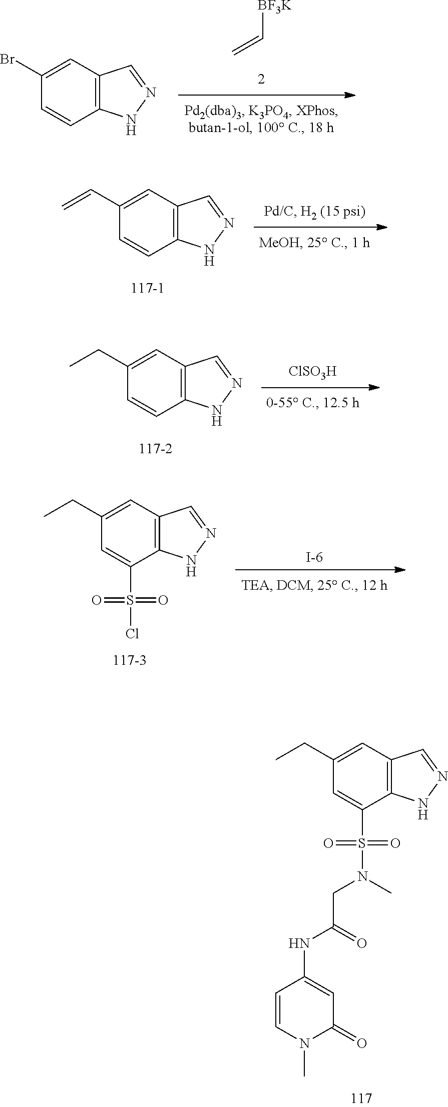

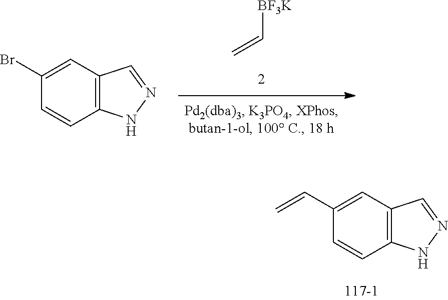

A mixture of 5-bromo-1H-indazole (5.00 g, 25.3 mmol), potassium trifluoro(vinyl)borate (5.10 g, 38.0 mmol), K₃PO₄ (10.77 g, 50.75 mmol), XPhos (483 mg, 1.02 mmol) and Pd₂(dba)₃ (232.38 mg, 253.77 umol) in buyan-1-ol (80 mL) was stirred at 100° C. for 18 hr under N₂. The reaction mixture was concentrated under reduced pressure. The residue was diluted with water (30 ml) and extracted with EtOAC (30 mL*3). The combined organic layers were dried over Na₂SO₄, filtered and concentrated under reduced pressure. The residue was purified by flash silica gel chromatography (ISCO®; 40 g SepaFlash® Silica Flash Column, Eluent of 0˜100% Ethyl acetate/Petroleum ether gradient @60 mL/min) to give 5-vinyl-1H-indazole (117-1) (5.21 g, 36.1 mmol) as a yellow solid.

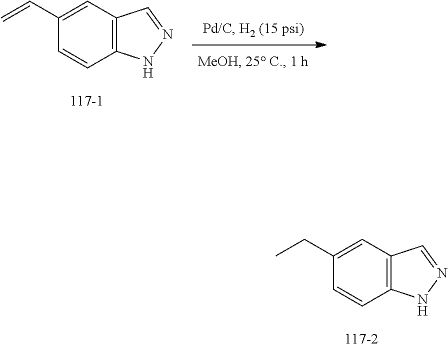

To a solution of 117-1 (1.00 g, 6.94 mmol) in MeOH (35 mL) was added Pd/C (200 mg, 10% purity). The suspension was degassed and purged with H₂ for 4 times. The mixture was stirred under H₂ (15 Psi) at 25° C. for 1 hr. The reaction mixture was filtered and concentrated under reduced pressure to give 5-ethyl-1H-indazole (117-2) (0.750 g, crude) was obtained as a white solid. No further purification was performed.

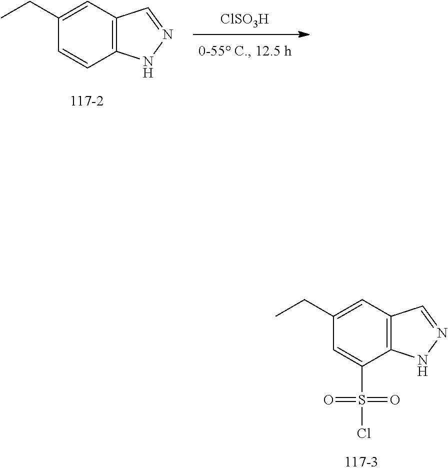

Compound 117-2 (0.750 g, 5.13 mmol) was added to chlorosulfonic acid (7.73 mL, 116 mmol) slowly at 0° C. under N₂. The mixture was stirred at 0° C. and warmed to 25° C. within 0.5 hr. Then the mixture was stirred at 55° C. for 12 hr under N₂. The reaction mixture was poured into 100 mL ice water, filtered and the filter cake was washed with THF/EtOAc=1/1 (200 mL) to a new bottle, the separated organic was dried over Na₂SO₄, filtered and concentrated under reduced pressure to give 5-ethyl-1H-indazole-7-sulfonyl chloride (117-3) (1.71 g, crude) as a yellow solid. No further purification was performed.

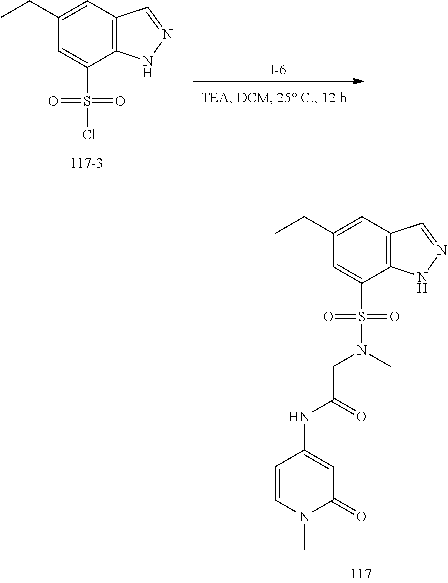

To a solution of I-6 (0.141 g, 0.458 mmol, TFA salt) in DCM (10 mL) was added TEA (0.231 g, 2.29 mmol) to PH>7. Then 117-3 (0.140 g, 0.457 mmol) was added. The mixture was stirred at 25° C. for 12 hr then concentrated under reduced pressure. The residue was purified by prep-HPLC (HCl condition), column: [Xtimate C18 100*30 mm*3 um; mobile phase: [water (0.04% HCl)-ACN]; B %: 17%-47%, 10 min] to give 2-[(5-ethyl-1H-indazol-7-yl)sulfonyl-methyl-amino]-N-(1-methyl-2-oxo-4-pyridyl)acetamide (117) (0.051 g, 0.13 mmol, 28% yield, HCl salt) as a white solid.

LCMS for 117: m/z 404.0 [M+H]⁺

¹H-NMR for 117: (400 MHz, METHANOL-d4) δ 1.34 (t, J=7.58 Hz, 3H) 2.86 (q, J=7.66 Hz, 2H) 2.95 (s, 3H) 3.71 (s, 3H) 4.22 (s, 2H) 6.90 (dd, J=7.34, 2.32 Hz, 1H) 7.36 (d, J=2.32 Hz, 1H) 7.77 (d, J=1.34 Hz, 1H) 7.89 (d, J-=7.34 Hz, 1H) 7.93-7.96 (m, 1H) 8.16 (s, 1H)

The following compound was synthesized in a similar manner as 117 using the reactant indicated.

| | | LCMS | |
|---|---|---|---|
| Example | Reactant | m/z | ¹H-NMR |
| 118 | potassium | 418.0 | (400 MHz, METHANOL-d4) δ 8.16 (s, |
| | allyl | | 1H), 7.92 (d, J = 1.3 Hz, 1H), 7.84 (d, |
| | (trifluoro) | | J = 7.3 Hz, 1H), 7.74 (d, J = 1.5 Hz, |
| | borate | | 1H), 7.28 (d, J = 2.2 Hz, 1H), 6.86 (dd, |
| | | | J = 2.3, 7.3 Hz, 1H), 4.21 (s, 2H), 3.69 |
| | | | (s, 3H)- 2.94 (s, 3H), 2.84-2.76 (m, |
| | | | 2H), 1.79-1.68 (m, 2H), 0.99 (t, J = 7.3 |
| | | | Hz, 3H) |

### Synthesis of 2-((2-cyano-N,5-dimethyl-1H-indole)-7-sulfonamido)-N-(1-methyl-2-oxo-1,2-dihydropyridin-4-yl)acetamide (Example 124)

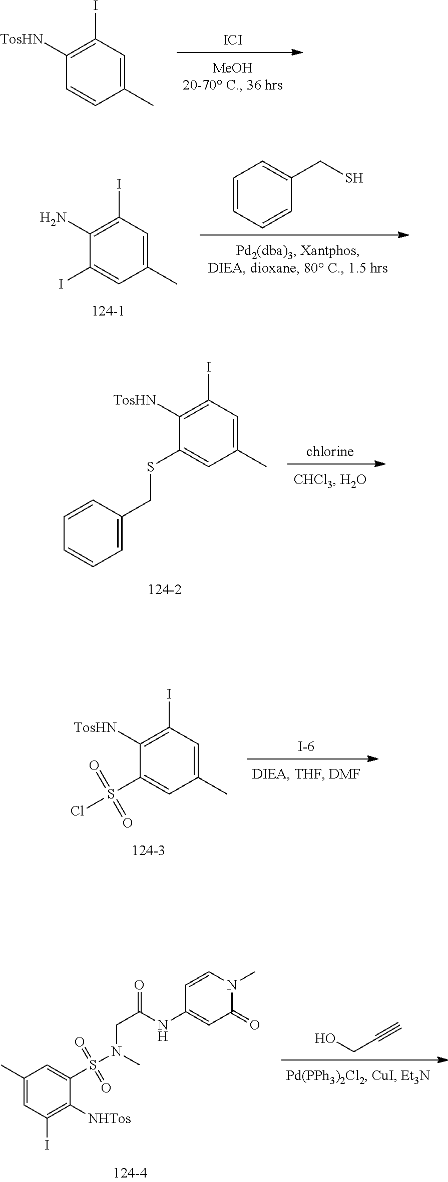

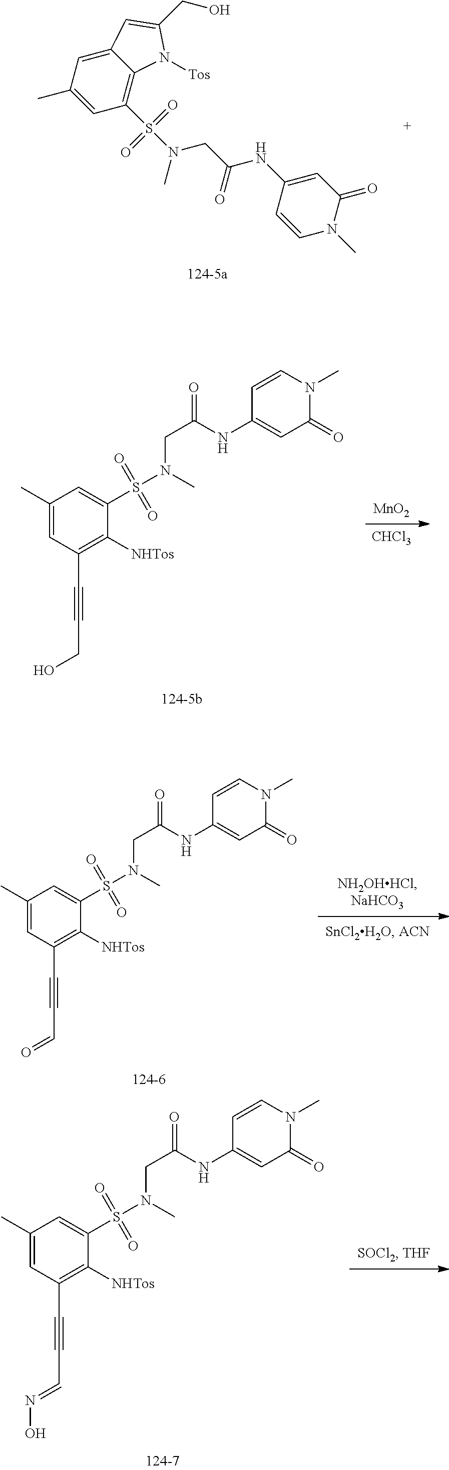

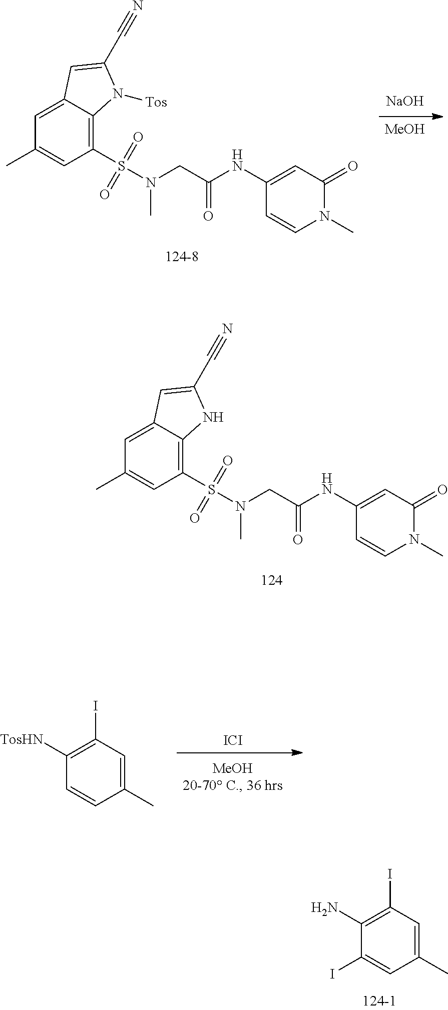

To a solution of 2-iodo-4-methylaniline (15.0 g, 64.4 mmol) in dichloromethane (150 mL) was added iodine chloride (3.77 mL, 73.9 mmol) at 0° C. The mixture was stirred at 25° C. for 12 hours. The mixture was poured into a saturated aqueous solution of sodium sulfite (500 mL) and adjusted the aqueous phase pH to ˜8 with sodium carbonate (s). The mixture was extracted with dichloromethane (100 mL*3). The combined organic layers were washed with brine (200 mL), dried over anhydrous sodium sulfate, filtered, and the filtrate was concentrated under reduced pressure. The residue was purified by column chromatography (SiO₂, petroleum ether/ethyl acetate=1/0˜1/0) and reversed-phase HPLC (0.1% TFA condition) to afford 2, 6-diiodo-4-methylaniline (124-1) (5.50 g, 15.3 mmol, 24% yield) as a white solid.

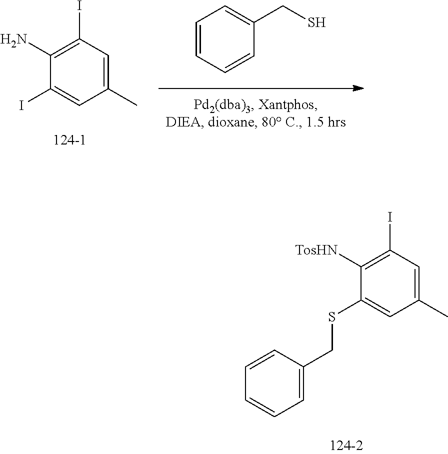

To a solution of 124-1 (4.30 g, 12.0 mmol) in dioxane (45 mL) was added diisopropylethylamine (6.25 mL, 35.9 mmol), Xantphos (0.970 g, 1.68 mmol) and Pd₂(dba)₃ (0.785 g, 0.857 mmol) at 25° C. Then a solution of phenylmethanethiol (1.2 mL, 10.2 mmol) in dioxane (8 mL) was added. The mixture was stirred at 85° C. for 1 hour. The mixture was poured into water (200 mL) and extracted with ethyl acetate (50 mL*3). The combined organic layers were washed with brine (20 mL), dried over anhydrous sodium sulfate, filtered, and the filtrate was concentrated under reduced pressure. The residue was purified by column chromatography (SiO₂, petroleum ether/ethyl acetate=1/0˜20/1) and reversed-phase HPLC (0.1% TFA condition) to afford 2-(benzylthio)-6-iodo-4-methylaniline (124-2) (1.95 g, 5.02 mmol, 42% yield) as brown oil.

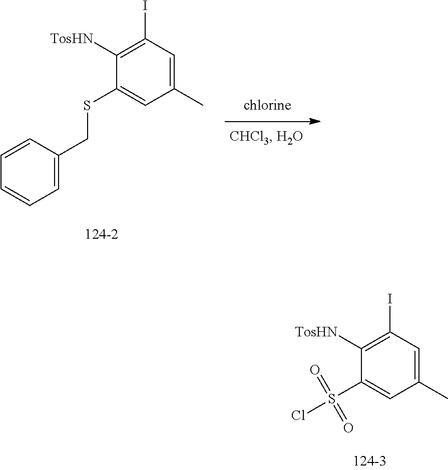

Chlorine gas was bubbled into a solution of 124-2 (0.600 g, 1.18 mmol) in chloroform (10 mL)/H₂O (5 mL) at 0° C. for 10 minutes. After excess Chlorine was purged with nitrogen. The mixture was poured into ice-water (w/w=1/1) (5 mL) and stirred for 5 minutes. The aqueous phase was extracted with dichloromethane (20 mL*3). The combined organic phase was washed with brine (10 mL*2), dried with anhydrous sodium sulfate, filtered, and the filtrate was concentrated under reduced pressure to give 3-iodo-5-methyl-2-(4-methylphenyl sulfonamido)benzene-1-sulfonyl chloride (124-3) (0.500 g, crude) as yellow gum.

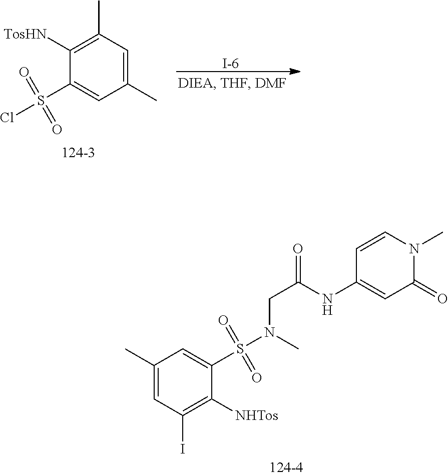

To a mixture of I-6 (0.357 g, 1.54 mmol), diisopropylethylamine (0.399 g, 3.09 mmol) in dichloromethane (5 mL) was added 124-3 (0.500 g, 1.03 mmol) under nitrogen. The mixture was stirred at 25° C. for 1 hour. The mixture was concentrated under reduced pressure to give a residue, which was purified by reversed-phase flash (0.1% TFA condition) to afford 2-(3-iodo-N,5-dimethyl-2-(4-methylphenylsulfonamido)phenylsulfonamido)-N-(1-methyl-2-oxo-1,2-dihydropyridin-4-yl)acetamide (124-4) (0.600 g, 0.866 mmol, 84.2% yield) as a yellow solid.

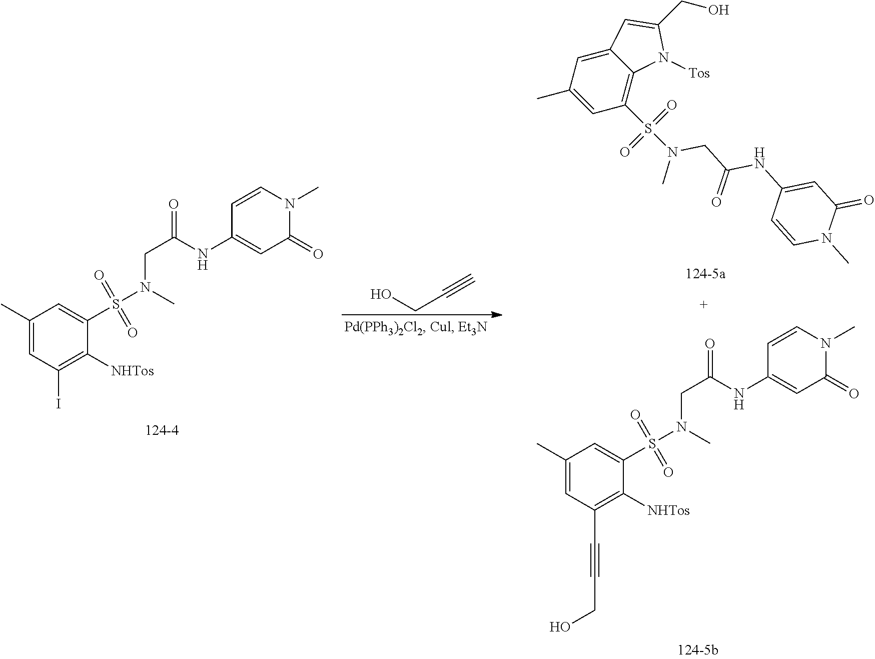

To a mixture of 124-4 (0.750 g, 1.16 mmol) and prop-2-yn-1-ol (3.00 g, 53.5 mmol), CuI (0.022 g, 0.116 mmol), triethylamine (0.588 g, 5.82 mmol) in N, N-dimethylformamide (5 mL) was added Pd(PPh₃)₄ (0.134 g, 0.116 mmol) in one portion at 25° C. under nitrogen. The mixture was stirred at 90° C. for 3 hours. The reaction mixture was concentrated under reduced pressure to give a residue, which was purified by reversed-phase flash (0.1% TFA/MeCN condition) to afford a mixture of 2-(2-(hydroxymethyl)-N,5-dimethyl-1-tosyl-1H-indole-7-sulfonamido)-N-(1-methyl-2-oxo-1,2-dihydropyridin-4-yl)acetamide (124-5a) and 2-(3-(3-hydroxyprop-1-yn-1-yl)-N,5-dimethyl-2-(4-methylphenylsulfonamido)phenylsulfonamido)-N-(1-methyl-2-oxo-1,2-dihydropyridin-4-yl)acetamide (124-5b) (0.600 g, 1.05 mmol, 90.0% yield, 5:5a=3:1 by ¹H NMR) as a yellow solid.

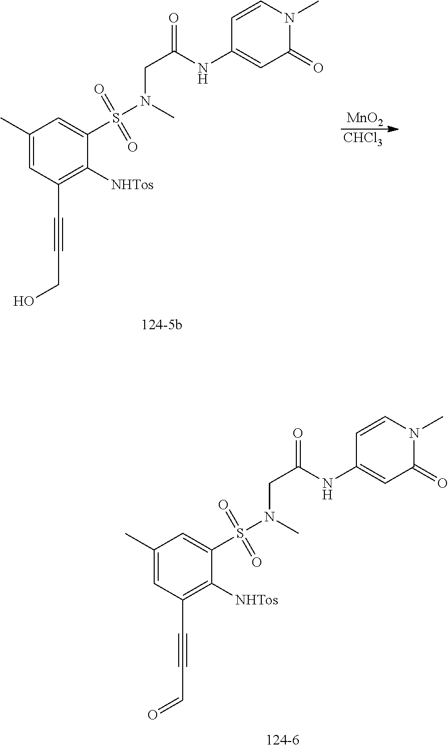

To the above 3:1 mixture of 124-5a (0.300 g, 0.523 mmol) and 124-5b (0.100 g, 0.174 mmol) in chloroform (20 mL) was added manganese dioxide (0.910 g, 10.4 mmol). The mixture was stirred at 70° C. for 5 hours. The mixture was filtered and the filtrate was concentrated under reduced pressure to give the crude product, which was purified by reversed-phase HPLC (0.5% TFA/MeCN condition) to afford 2-(N,5-dimethyl-2-(4-methylphenylsulfonamido)-3-(3-oxoprop-1-yn-1-yl)phenylsulfonamido)-N-(1-methyl-2-oxo-1,2-dihydropyridin-4-yl)acetamide (124-6) (0.089 g, 0.139 mmol, 89.0% yield) as a yellow solid.

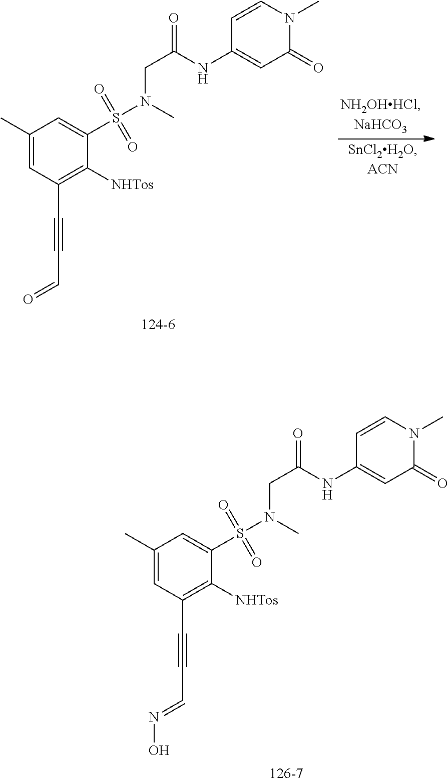

To a mixture of 124-6 (0.070 g, 0.122 mmol), sodium bicarbonate (0.051 g, 0.613 mmol), and SnCl₂·2H₂O (0.027 g, 0.122 mmol) in acetonitrile (3 mL) was added hydroxylamine hydrochloride (0.042 g, 0.613 mmol) under nitrogen. The mixture was stirred at 80° C. for 1 hours then concentrated under reduceIressure22lydroxyaminoE)-2-(3-(3-(hydroxyimino)prop-1-yn-1-yl)-N,5-dimethyl-2-(4-methylphenylsulfonamido)phenylsulfonamido)-N-(1-methyl-2-oxo-1,2-dihydropyridin-4-yl)acetamide (124-7) (0.070 g, crude) as a yellow solid.

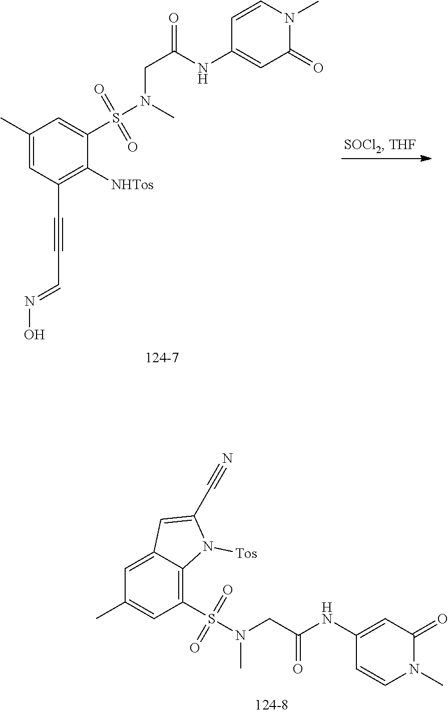

To a mixture of 124-7 (0.070 g, 0.119 mmol) in tetrahydrofuran (1 mL) was added SOCl₂ (0.028 g, 0.239 mmol) in one portion at 25° C. under nitrogen. The mixture was stirred at 25° C. for 1 hour. The reaction mixture was partitioned between water (10 mL) and ethyl acetate (50 mL). The organic phase was separated, washed with ethyl acetate (50 mL*3), dried over anhydrous sodium sulfate, filtered, and the filtrate was concentrated under reduced pressure to give a residue, which was purified by prep-TLC (SiO₂, ethyl acetate) to afford 2-(2-cyano-N,5-dimethyl-1-tosyl-1H-indole-7-sulfonamido)-N-(1-methyl-2-oxo-1,2-dihydropyridin-4-yl)acetamide (124-8) (0.040 g, 0.047 mmol, 39.9% yield) as a yellow solid.

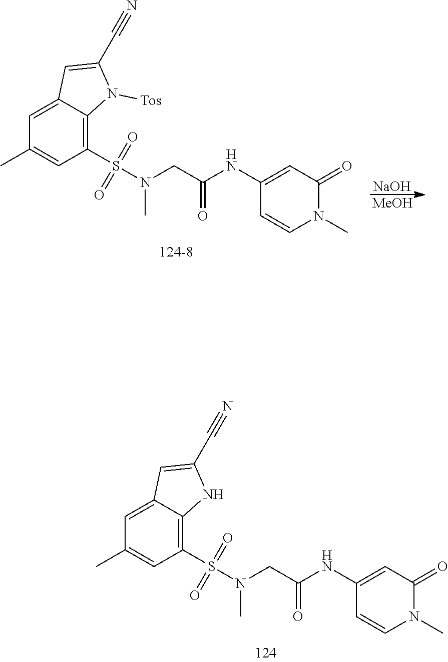

To a mixture of 124-8 (0.030 g, 0.035 mmol) in methyl alcohol (1.5 mL) was added sodium hydroxide (0.028 g, 0.715 mmol) and the reaction mixture was stirred at 25° C. for 1 hour. The reaction mixture was filtered and the filtrate was concentrated under reduced pressure to give a residue, which was purified by prep-TLC (SiO₂, dichloromethane:methyl alcohol=15:1) then reversed-phase HPLC (0.1% NH₃·H₂O/MeCN) to afford 2-(2-cyano-N,5-dimethyl-1H-indole-7-sulfonamido)-N-(1-methyl-2-oxo-1,2-dihydropyridin-4-yl)acetamide (124) (10.5 mg, 0.023 mmol, 49.9% yield) as a yellow solid.

LCMS of 124: m/z=413.9 [M+H]⁺

¹H-NMR of 124 (CD₃OD, 400 MHz) δ 7.78 (s, 1H), 7.67 (s, 1H), 7.58 (d, J=7.6 Hz, 1H), 7.27 (s, 1H), 6.82 (d, J=2.4 Hz, 1H), 6.62 (dd, J=2.4, 7.2 Hz, 1H), 4.13 (s, 2H), 3.51 (s, 3H), 2.89 (s, 3H), 2.49 (s, 3H)

### Synthesis of 2-((N,5-dimethyl-1H-indazole)-7-sulfonamido)-N-((1-phenyl-1H-1,2,3-triazol-4-yl)methyl)acetamide (Example 125)

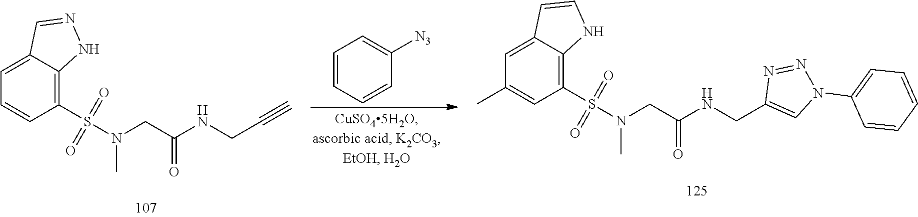 [SMILES: C#CCNC(=O)CN(C)S(=O)(=O)c1cccc2cn[nH]c12 ; [N-]=[N+]=Nc1ccccc1 ; Cc1cc(S(=O)(=O)N(C)CC(=O)NCc2cn(-c3ccccc3)nn2)c2[nH]ccc2c1]

To a solution of 107 (0.035 g, 0.109 mmol) and azidobenzene (0.016 g, 0.134 mmol) in ethyl alcohol (4 mL) was added a solution of copper(II)sulfate pentahydrate (0.004 g, 0.016 mmol), (R)-5-((S)-1,2-dihydroxyethyl)-3,4-dihydroxyfuran-2(5H)-one (0.005 g, 0.028 mmol and potassium carbonate (0.012 g, 0.087 mmol) in water (1 mL). The mixture was stirred at 20° C. under nitrogen for 12 hours. The mixture was poured into water (10 mL) and extracted with ethyl acetate (10 mL*3). The combined organic layers were washed with brine (20 mL), dried over anhydrous sodium sulfate, filtered, and the filtrate was concentrated under reduced pressure. The crude product was purified by prep-HPLC (column: Waters Xbridge 150*25 mm*5 um; mobile phase: [water (10 mM NH₄HCO₃)-ACN]; B %: 31%-61%, 10 min) to afford 2-((N,5-dimethyl-1H-indazole)-7-sulfon amido)-N-((1-phenyl-1H-1,2,3-triazol-4-yl)methyl)acetamide (125) (0.012 g, 0.027 mmol, 17% yield) as a white solid.

LCMS of 125: m/z=440.1 [M+H]⁺

¹H-NMR of 125 (DMSO-d6, 400 MHz) δ 13.35 (br. s, 1H), 8.69 (t, J=5.2 Hz, 1H), 8.60 (s, 1H), 8.18 (s, 1H), 7.93-7.85 (m, 3H), 7.65-7.57 (m, 3H), 7.52-7.46 (m, 1H), 4.42 (d, J=5.2 Hz, 2H), 3.92 (s, 2H), 2.75 (s, 3H), 2.47 (s, 3H)

### Synthesis of 2-((N,5-dimethyl-1H-benzo[d][1,2,3]triazole)-7-sulfonamido)-N-(1-methyl-2-oxo-1,2-dihydropyridin-4-yl)acetamide (Example 132)

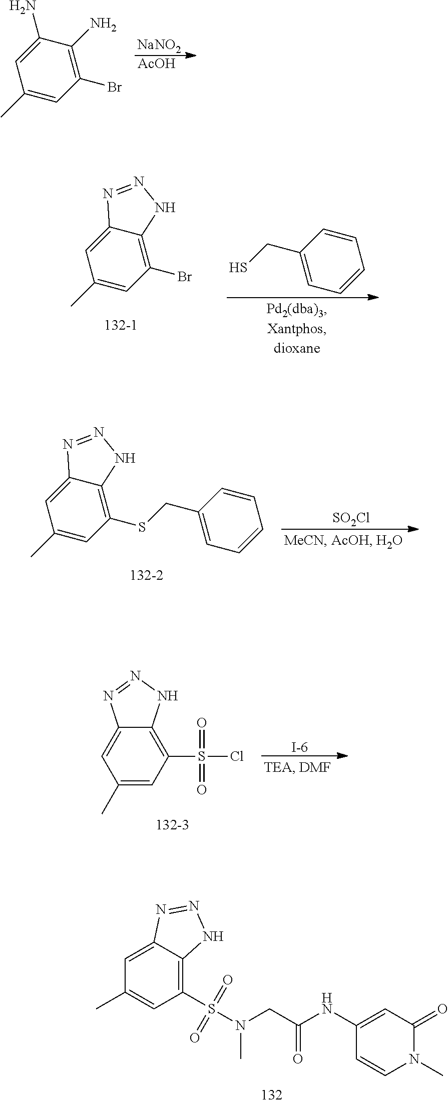

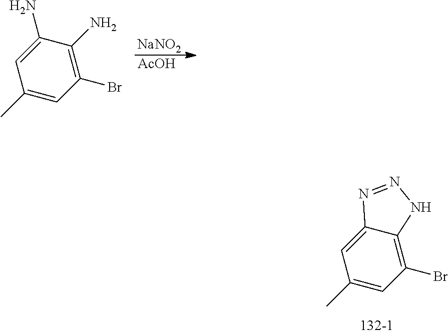

To a solution of 3-bromo-5-methylbenzene-1,2-diamine (1.00 g, 4.97 mmol) in acetic acid (10 mL) at 0° C. was added a solution of sodium nitrite (0.515 g, 7.46 mmol) in water (4 mL). Then the mixture was stirred at 0° C. for 5 minutes. The mixture was quenched with water (20 mL), causing a solid to precipitate out and the solid was collected by filtration to afford 7-bromo-5-methyl-1H-benzotriazole (132-1) (1.01 g, 4.78 mmol, 96.10% yield) as a yellow solid. No further purification was performed.

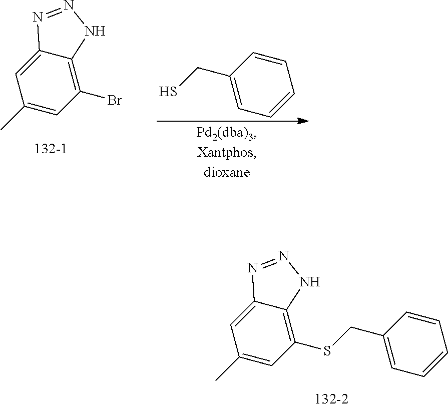 [SMILES: Cc1cc(Br)c2[nH]nnc2c1 ; SCc1ccccc1 ; Cc1cc(SCc2ccccc2)c2[nH]nnc2c1]

To a solution of 132-1 (1.00 g, 4.72 mmol) in dioxane (10 mL) was added diisopropyl ethylamine (2.46 mL, 14.2 mmol), Xantphos (1.09 g, 1.89 mmol) and Pd₂(dba)₃ (0.864 g, 0.944 mmol). The mixture was degassed and purged with nitrogen for three times. To the above mixture was added benzyl mercaptane (1.11 mL, 9.43 mmol) and the mixture was stirred at 80° C. for 12 hours under a nitrogen atmosphere. The mixture was filtered and the filtrate was concentrated under reduced pressure. The residue was purified by column chromatography (SiO₂, petroleum ether/ethyl acetate=3/1 to 1/1) to afford 7-(benzylthio)-5-methyl-1H-benzo[d][1,2,3]triazole (132-3) (0.300 g, 1.13 mmol, 24% yield) as a yellow solid. No further purification was performed.

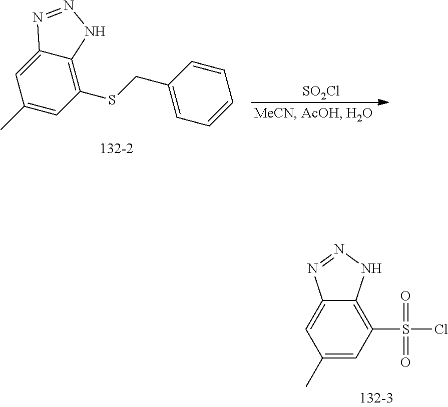 [SMILES: Cc1cc(SCc2ccccc2)c2[nH]nnc2c1 ; "SO2Cl" ; Cc1cc(S(=O)(=O)Cl)c2[nH]nnc2c1]

To 132-3 (0.050 g, 0.196 mmol) in a mixture of acetonitrile (1 mL), water (0.1 mL) and acetic acid (0.2 mL) was added sulfuryl chloride (0.080 g, 0.587 mmol) drop-wise at 0° C. The mixture was stirred at 0° C. for 0.5 hour then diluted with 20 mL of ice water. The mixture was extracted with ethyl acetate (20 mL*5) and the combined organic layer was washed with brine (30 mL*3), dried over anhydrous sodium sulfate, filtered, and the filtrate was concentrated under reduced pressure to afford 5-methyl-1H-benzo[d][1,2,3]triazole-7-sulfonyl chloride (132-3) (0.050 g, crude) as a white solid which was used directly without further purification.

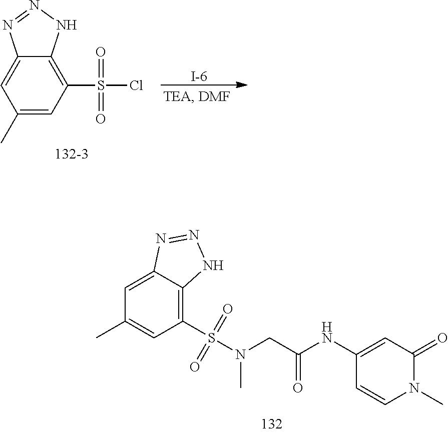

To a mixture of the crude 132-3 (0.050 g, 0.216 mmol) in dimethylformamide (3 mL) was added triethylamine (0.180 mL, 1.30 mmol), followed by I-6 (0.050 g, 0.216 mmol) at 0° C., and the mixture was stirred at 0° C. for 10 minutes. The mixture was concentrated under reduced pressure and the residue was purified by prep-HPLC (column: Zhongpu RD-C₁₈ 150*25 mm*3 um; mobile phase: [water (0.05% HCl)-ACN]; B %: 9%-42%, 10 min) followed by prep-HPLC (column: 3_Phenomenex Luna C₁₈ 75*30 mm*3 um; mobile phase: [water (0.05% HCl)-ACN]; B %: 13%-33%, 7 min) to afford 2-(N,5-dimethyl-1H-benzo[d][1,2,3]triazole-7-sulfonamido)-N-(1-methyl-2-oxo-1,2-dihydropyridin-4-yl)acetamide (132) (0.059 g, 0.150 mmol, 69.7% yield) as a white solid.

LCMS of 132: m/z 391.3 [M+H]⁺

¹H NMR of 132: (DMS-d₆, 400 MHz): δ 10.39-10.15 (m, —H), 8.02 (s, 1H), 7.-6-7.68 (m, 1H), 7.64-7.55 (m, 1H), 7-37-7.20 (m, 1H), 6.-2-6.56 (m, 1H), 6.41-6.26 (m, —H), 4.24 (s, 2H), 3.39-3.31 (m, 3H), 2.89 (s, 3H), 2.54 (s, 3H)

### Synthesis of 2-((N,5-dimethyl-3-oxo-2,3-dihydro-1H-indazole)-7-sulfonamido)-N-(1-methyl-2-oxo-1,2-dihydropyridin-4-yl)acetamide (Example 133)

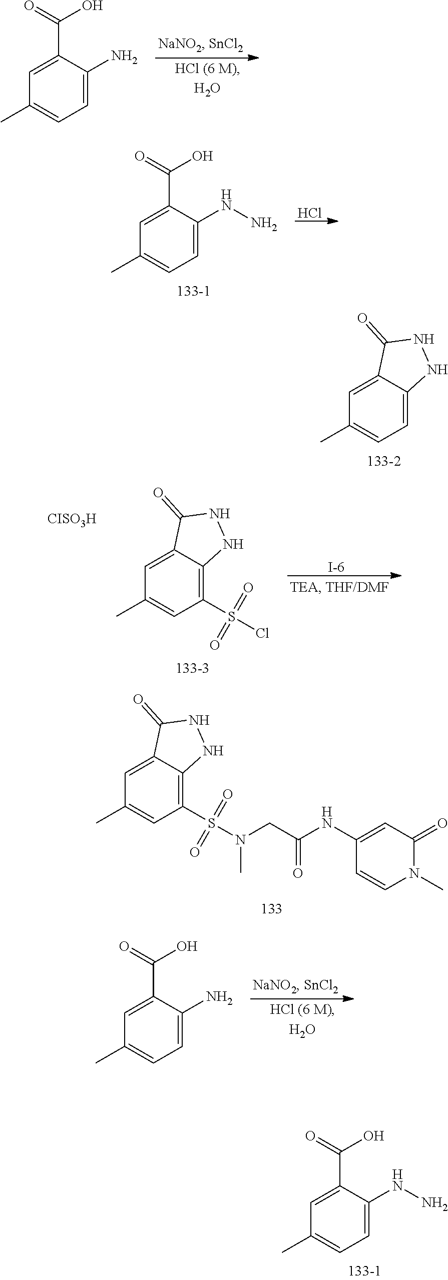

A solution of 2-amino-5-methylbenzoic acid (1.00 g, 6.62 mmol) in hydrochloric acid (6 M, 10 mL) was cooled down to −5° C. Then sodium nitrite (0.502 g, 7.28 mmol) in water (2 mL) was added drop wise. The mixture was stirred at −5° C. for 1 hour. Then a mixture of tin dichloride dihydrate (2.99 g, 13.2 mmol) dissolved in hydrochloric acid (6 M, 2 mL) was added. The mixture was stirred at −5-25° C. for 2 hours. The mixture was filtered and the filtrate cake was rinsed with water (10 mL) then dried under reduced pressure to afford 2-hydrazinyl-5-methylbenzoic acid as the hydrochloric salt (133-1) (1.00 g, 4.93 mmol, 74.6% yield) as a white solid. No further purification was performed.

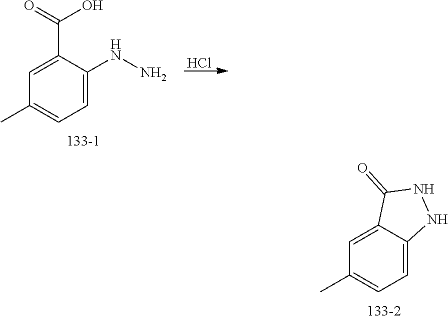

A solution of 133-1 (1.00 g, 4.93 mmol) as a hydrochloric salt in hydrochloric acid (12 M, 8.2 mL) was stirred at 80° C. for 16 hours. The pH of the mixture was regulated with a solution of sodium hydroxide to approximately pH˜7 then extracted with ethyl acetate (50 mL*3). The combined organic layers were washed with brine (100 mL), dried over anhydrous sodium sulfate, filtered, the filtrate was concentrated under reduced pressure. The residue was purified by prep-TLC (petroleum ether/ethyl acetate=0/1) to afford 5-methyl-1H-indazol-3(2H)-one (133-2) (0.150 g, 0.983 mmol, 19.9% yield) as a yellow solid.

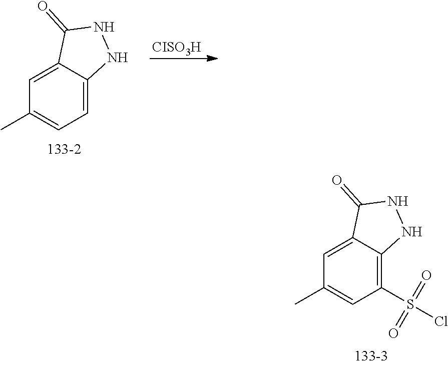

A solution of sulfurochloridic acid (2 mL, 30.1 mmol) was cooled down to 0° C., then 133-2 (0.150 g, 1.01 mmol) was added in batches. The reaction was stirred at 0-25° C. for 2 hours. The mixture was poured into ice water (50 mL) portion wise and extracted with ethyl acetate (20 mL*3). The combined organic layers were washed with brine (50 mL), dried over anhydrous sodium sulfate, filtered, the filtrate was concentrated under reduced pressure to afford 5-methyl-3-oxo-2,3-dihydro-1H-indazole-7-sulfonyl chloride (4) (0.065 g, 0.169 mmol, 16.7% yield) as a yellow solid. No further purification was performed.

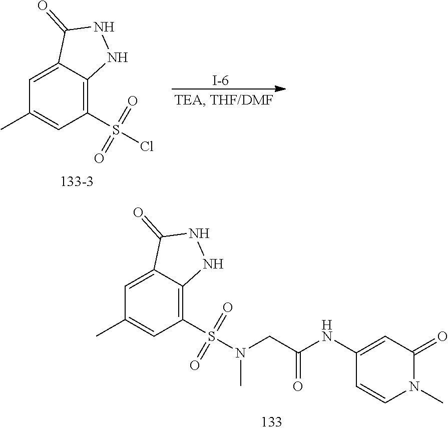

To a solution of I-6 as the hydrochloric acid salt (0.045 g, 0.194 mmol) in dimethyl formamide (1 mL) was added triethylamine (0.220 mL, 1.58 mmol) followed by a solution of 133-3 (0.070 g, 0.186 mmol) in tetrahydrofuran (0.5 mL) at 0° C. The mixture was stirred at 0° C. for 1 hour. The reaction mixture was quenched with water (1 mL), then the resulting mixture was purified by prep-HPLC (column: Zhongpu RD-Cis 150*25 mm*3 um; mobile phase: [water (0.1% TFA)-ACN]; B %: 13%-35%, 10 min) to afford 2-((N,5-dimethyl-3-oxo-2,3-dihydro-1H-indazole)-7-sulfonamido)-N-(1-methyl-2-oxo-1,2-dihydropyridin-4-yl)acetamide (133) (0.007 g, 0.015 mmol, 8% yield) as a white solid.

LCMS of 133: m/z 406.1 [M+H]⁺

¹H-NMR of 133: (DMSO-d₆+D₂O, 400 MH−) δ 7.73 (s, 1H), 7.59-7.53 (m, 2H), 6.65 (d, J=2.4 Hz, 1H), 6.35 (dd, J=7.6, 2.4 Hz, 1H), 4.05 (s, 2H), 3.34 (s, 3H), 2.79 (s, 3H), 2.39 (s, 3H)

### Synthesis of 2-((N,5-dimethyl-1H-indazole)-7-sulfonamido)-N-(4,5,6,7-t229ydroxyarothiazolo[4,5-c]pyridin-2-yl)acetamide (Example 136)

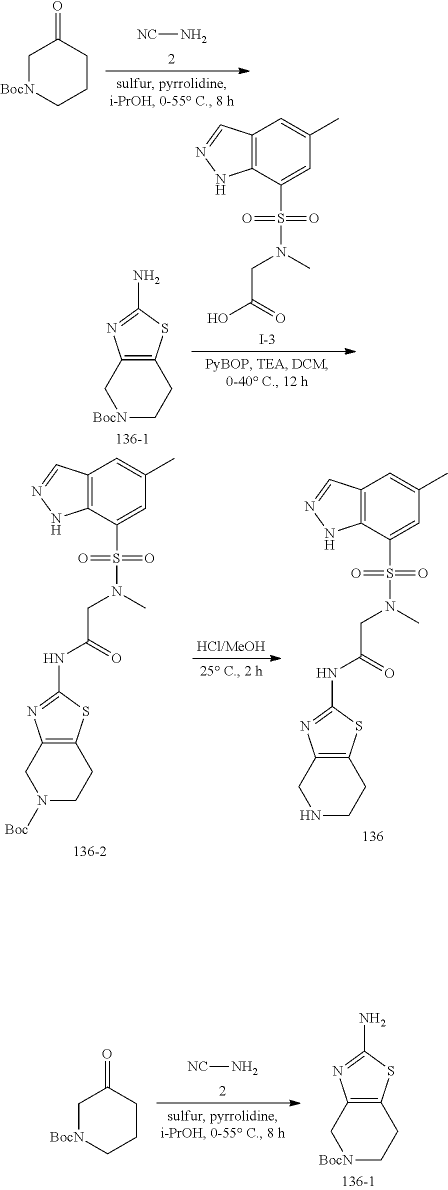

To a solution of tert-butyl 3-oxopiperidine-1-carboxylate (5.00 g, 25.0 mmol) in i-PrOH (50 mL) was added pyrrolidine (2.20 mL, 26.3 mmol) and the mixture was stirred at 55° C. for 2 hr. Then elemental sulfur (0.805 g, 25.1 mmol) was added in one portion, followed by dropwise addition of a solution of cyanamide (1.11 g, 26.3 mmol) in i-PrOH (15 mL) at 0° C. The resulting mixture was stirred at 20° C. for 6 hr then concentrated in vacuum. The residue was diluted with EtOAc (60 mL), washed with sat. Na₂CO₃ (50 mL) followed by brine (50 mL) then dried over Na₂OS₄, filtered, and concentrated under vacuum. The residue was purified by flash silica gel chromatography (ISCO®; 40 g SepaFlash® Silica Flash Column, Eluent of 0-100% Ethyl acetate/Petroleum ethergradient@45 mL/min) to provide tert-butyl 2-amino-6,7-dihydrothiazolo[4,5-c]pyridine-5(4H)-carboxylate (136-1) (1.30 g, 20.2% yield) as a yellow solid.

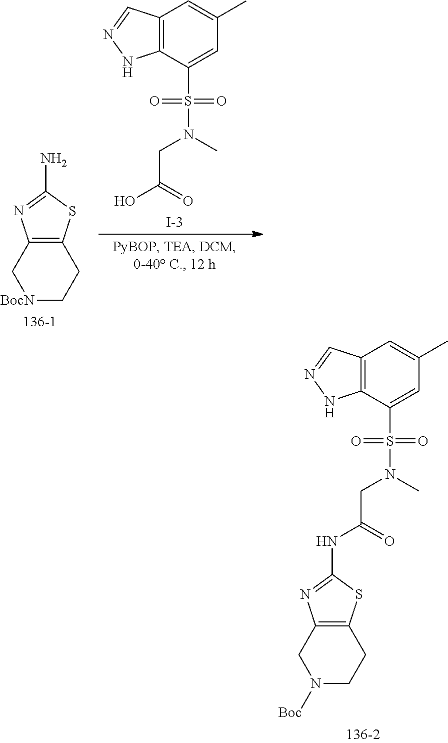

To a solution of 136-1 (0.200 g, 0.783 mmol), I-3 (0.148 g, 0.522 mmol) and PYBOP (0.543 g, 1.04 mmol) in DCM (10 mL) was added TEA (0.363 mL, 2.61 mmol) at 0° C. under N₂. The mixture was stirred at 40° C. for 12 hr under N₂ then the reaction mixture was concentrated under vacuum. The residue was purified by flash silica gel chromatography (ISCO®; 12 g SepaFlash® Silica Flash Column, Eluent of 0˜100% Ethyl acetate/Petroleum ethergradient@40 mL/min) to provide tert-butyl2-(2-(N,5-dimethyl-1H-indazole-7-sulfonamido) acetamido)-6,7-dihydrothiazolo[4,5-c]pyridine-5(4H)-carboxylate (136-2) (0.200 g, 73.5% yield) as a yellow oil.

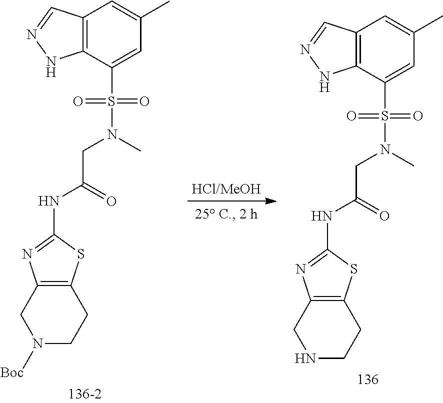

A mixture of 136-2 (0.200 g, 0.384 mmol) in HCl/MeOH (5 mL) was stirred at 25° C. for 2 hr. The reaction mixture was concentrated under vacuum to give a residue. The residue was triturated with MeOH (8 mL) at 25° C. for 1 h. Then the mixture was filtered and the filter cake was dried under vacuum. The residue was purified by prep-HPLC (HCl condition) [column: Xtimate C18 100*30 mm*3 um; mobile phase: [water (0.04% HCl)-ACN]; B %: 12%-36%, 8 min] to provide 2-(N,5-dimethyl-1H-indazole-7-sulfonamido)-N-(4,5,6,7-tetrahydrothiazolo[4,5-c]pyridin-2-yl)acetamide (136) (0.045 g, 27% yield, HCl salt) as a yellow solid.

LCMS of 136: m/z 421.0 [M+H]⁺

¹H-NMR for 136: (400 MHz, DMSO-d6) δ 12.28 (s, 1H), 9.40 (br s, 2H), 8.20 (s, 1H), 7.91 (s, 1H), 7.62 (d, J=1.1 Hz, 1H), 4.25 (s, 2H), 4.16 (br s, 2H), 2.98 (br t, J=5.7 Hz, —H), 2.87 (s, 3H), 2.56-2.53 (m, 2H), 2.47 (s, 3H)

### Synthesis of 2-((N,5-dimethyl-1H-indazole)-7-sulfonamido)-N-(5-methyl-4,5,6,7-t23lydroxyarothiazolo[4,5-c]pyridin-2-yl)acetamide (Example 137)

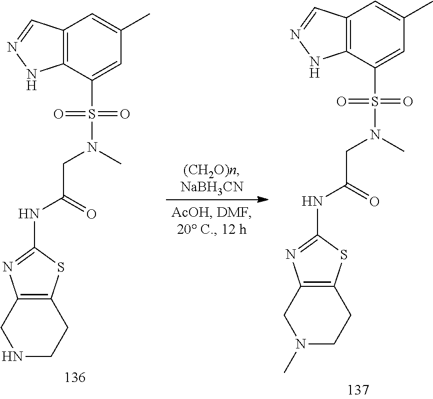

To a solution of 136 (0.080 g, 0.19 mmol) in DMF (2.5 mL) was added AcOH (0.022 mL, 0.38 mmol) and formaldehyde (0.017 g, 0.57 mmol). The mixture was stirred at 20° C. for 1 hr then NaBH₃CN (0.036 g, 0.57 mmol) was added to the mixture and the resulting mixture was stirred at 20° C. for 11 hr. The reaction mixture was quenched with HCl (0.5 M, 1 mL) then the mixture was adjusted to approximately pH to 8 by addition of a saturated aqueous solution of NaHCO₃. The mixture was extracted with EtOAc (10 mL*3) and the combined organic layers were dried over Na₂SO₄, filtered, 232ydrooncentrated under vacuum. The 232ydrodue was purified by prep-HPLC (HCl condition) [column: Xtimate C18 100*30 mm*3 um; mobile phase: [water (0.04% HCl)-ACN]; B %: 12%-35%, 8 min] to give 2-(N,5-dimethyl-1H-indazole-7-sulfonamido)-N-(5-methyl-4,5,6,7-tetrahydrothiazolo[4,5-c]pyridin-2-yl)acetamide (137) (0.022 g, 24% yield, HCl salt) as a white solid.

LCMS of 137: m/z 435.0 [M+H]⁺

¹H-NMR for 137: (40-MHz, DMSO-d6) δ 13.46-12.80 (m, 1H), 12.34 (br s, 1H), 11.26 (br s, 1H), 8.20 (s, 1H), 7.91 (s, 1H), 7.62 (s, 1H), 4.38 (br d, J=14.9 Hz, 1H), 4.25 (s, 2H), 4.15 (br dd, J=7.0, 14.9 Hz, 1H), 3.67 (br d, J=10.0 Hz, 1H), 3.34 (br d-J=9.0 Hz, 1H), 3.18-3.00 (m, 2H), 2.91 (br d, J=3.9 Hz, 3H), 2.87 (s, 3H), 2.47 (s, 3H)

### Synthesis of (S)-1-((5-methyl-1H-indazol-7-yl)sulfonyl)-N—((S)-tetrahydro-2H-pyran-3-yl)azetidine-2-carboxamide (141) and (S)-1-((5-methyl-1H-inIol-7-yl)sulfonyl)-N—((R)-tetrahydro-2H-pyran-3-yl)azetidine-2-carboxamide (Example 141 and Example 142)

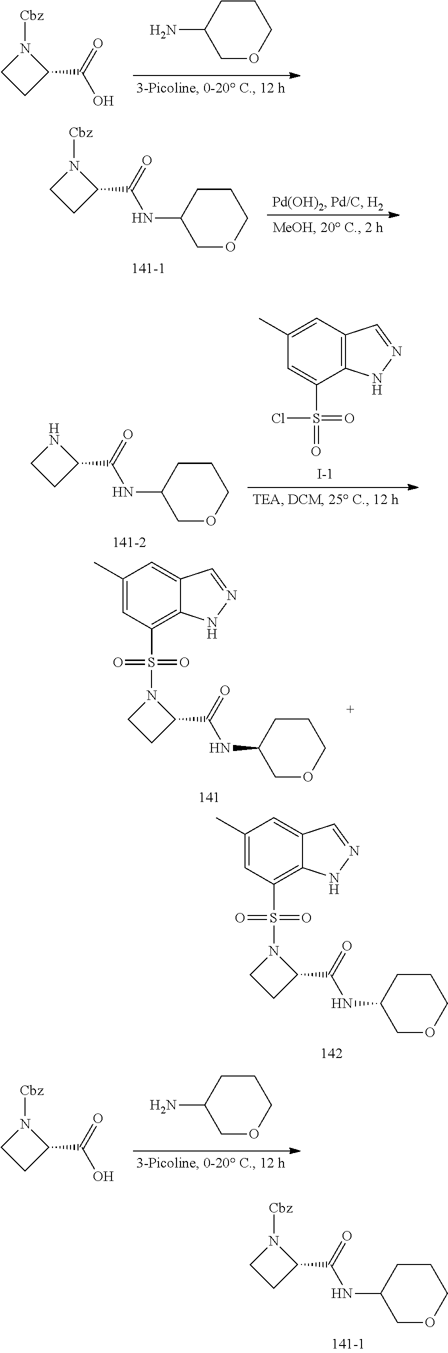

To a solution of (S)-1-((benzyloxy)carbonyl)azetidine-2-carboxylic acid (0.730 g, 3.10 mmol) and tetrahydropyran-3-amine (2) (0.200 g, 1.98 mmol) in 3-picoline (20 mL) was added MsCl (0.508 mL 6.56 mmol) at 0 C. The mixture was stirred at 20° C. for 12 hr under N₂ then the reaction mixture was quenched with water (10 mL) and extracted with EtOAc (20 mL*3). The combined organic layers were washed with brine (20 mL), dried over Na₂SO₄, filtered, and concentrated under reduced pressure to give a residue. The residue was purified by column chromatography (SiO₂, Petroleum ether/Ethyl acetate=1/0 to 0/1) to provide benzyl (2S)-2-(tetrahydropyran-3-ylcarbamoyl)azetidine-1-carboxylate (141-1) (0.690 g, crude) as a yellow oil. No further purification was performed.

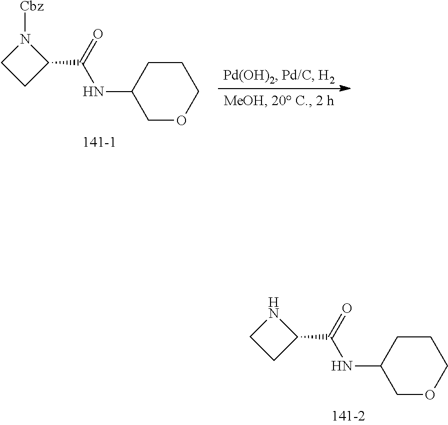 [SMILES: O=C(NC1CCCOC1)[C@@H]1CCN1C(=O)OCc1ccccc1 ; O=C(NC1CCCOC1)[C@@H]1CCN1]

To a mixture of Pd(OH)₂ (0.120 g, 0.854 mmol) and Pd/C (0.24 g, 10% on Carbon) in MeOH (5 mL) was added 141-1 (0.600 g, 1.88 mmol). The mixture was stirred at 20° C. for 2 hr under H₂ at 15 psi. The reaction mixture was filtered and concentrated under reduced pressure to provide (2S)—N-(tetrahydro-2H-pyran-3-yl)azetidine-2-carboxamide (141-2) (0.310 g, 89.2% yield) as a yellow solid. No further purification was performed.

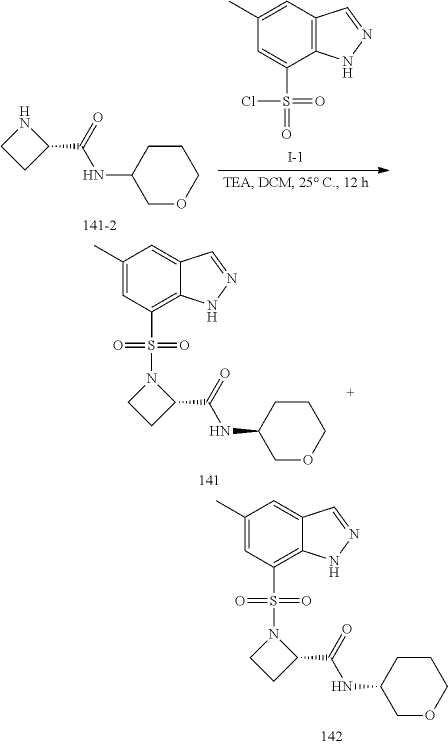

To a solution of 141-2 (0.31 g, 1.68 mmol) in DCM (5 mL) was added TEA (702 mL 5.05 mmol) and I-1 (0.465 g, 2.02 mmol). The mixture was stirred at 25° C. for 12 hr then concentrated under reduced pressure to give a residue. The residue was purified by prep-HPLC (neutral condition) then separated by Chiral SFC (Instrument: Waters SFC150 AP preparative SFC; Column: Chiralcel OD, 250*30 mm i.d. 10 um; Mobile phase: A for CO₂ and B for MeOH (0.1% NH₃H₂O); Gradient: B %=35% isocratic elution mode; Flow rate: 65 g/min; Column temperature: 35° C.; System back pressure: 120 bar) to give (2S)-1-[(5-methyl-1H-indazol-7-yl)sulfonyl]-N-[(3S)-tetrahydropyran-3-yl]azetidine-2-carboxamide (141) (first eluting peak, 0.0284 g, 33.4% yield) as a yellow solid and (2S)-1-[(5-methyl-1H-indazol-7-yl)sulfonyl]-N-[(3R)-tetrahydropyran-3-yl]azetidine-2-carboxamide (142) (second eluting peak, 0.0159 g, 18.7% yield) as a yellow solid. The relative stereochemistry at the 3-amino tetrahydropyran position was arbitrarily assigned.

LCMS for 141: m/z 379.1 [M+H]⁺

¹H-NMR for 141: (400 MHz, METHANOL-d4) δ 8.15 (s, 1H), 7.96 (s, 1H), 7.75 (s, 1H), 4.63 (t-J=8.6 Hz, 1H), 3.-0-3.76 (m, 3H), 3.-5-3.67 (m, 2H), 3.-6-3.60 (m, 1H), 3.59-3.54 (m, —H), 2.54 (s, 3H), 2.-1-2.24 (m, 2H), 1.-8-1.89 (m, 1H), 1.-4-1.74 (m, 1H), 1.-4-1.74 (m, 1H), 1.68-1.57 (m, 2H)

LCMS for 142: m/z 379.1 [M+H]⁺

¹H-NMR for 142: (400 MHz, METHANOL-d4) δ 8.15 (s, 1H), 7.96 (s, 1H), 7.74 (s, 1H), 4.63 (t-J=8.6 Hz, 1H), 3.-0-3.79 (m, 3H), 3.-4-3.64 (m, 2H), 3.-1-3.54 (m, 1H), 3.40-3.35 (m, —H), 2.54 (s, 3H), 2.-3-2.24 (m, 2H), 1.-2-1.85 (m, 1H), 1.-3-1.72 (m, 1H), 1.65-1.57 (m, 2H)

### Synthesis of 2-((N-ethyl-5-methyl-1H-indazole)-7-sulfonamido)-N-(4-(1-hydroxy-2-methylpropan-2-yl)-3,4-dihydro-2H-benzo[b][1,4]oxazin-7-yl)acetamide (Example 145)

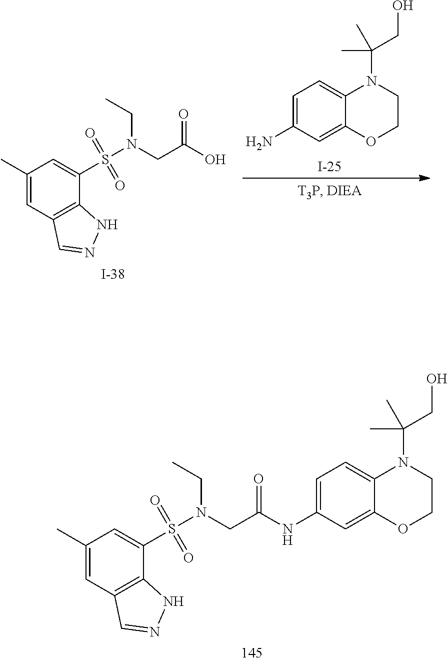

To a solution of I-25 (0.045 g, 0.140 mmol) and I-38 (0.015 g, 0.050 mmol) in N, N-dimethylformamide (0.5 mL) at 0° C. was added N, N-diisopropylethylamine (0.04 mL, 0.230 mmol) and T₃P (0.06 mL, 0.101 mmol) with 50% purity in ethyl acetate drop wise. The mixture was stirred at 25° C. for 12 hours. The mixture was concentrated under reduced pressure. The residue was purified by prep-HPLC (column: Waters Xbridge 150*25 mm*5 um; mobile phase: [water (10 mM NH₄HCO₃)-ACN]; B %: 30%-60%, 9 min) to afford 2-((N-ethyl-5-methyl-1H-indazole)-7-sulfonamido)-N-(4-(1-hydroxy-2-methylpropan-2-yl)-3,4-dihydro-2H-benzo[b][1,4]oxazin-7-yl)acetamide (145) (0.010 g, 20% yield) as a purple solid.

LCMS of 145: m/z 502.1 [M+H]⁺

¹H NMR of 145: (DMSO-d₆, 400 MHz): δ 9.88 (br. s, 1H), 8.17 (s, 1H), 7.89 (s, —H), 7.66 (s, 1H), 7.-8-6.66 (m, 3H), 4.-8-4.71 (m, 1H), 4.-8-4.13 (m, 4H), 3.-8-3.45 (m, 2H), 3.-7-3.25 (m, 2H), 3.23-3.17 (m, 2H), 2.44 (s, 3H), 1.22 (s, 6H), 0.95 (t, J=7.2 Hz, 3H)

The following compounds were synthesized in a similar manner as 145 using the reactants indicated.

| | | LCMS | |
|---|---|---|---|
| Example | Reactant | m/z | ¹H-NMR |
| 155 | I-18 | 486.2 | (400 MHz, METHANOL-d4) δ 8.12 (s, |
| | | | 1H), 7.88 (s, 1H), 7.79 (s, 1H), 7.01 (d, |
| | | | J = 2.4 Hz, 1H), 6.89 (dd, J = 2.3, 8.7 |
| | | | Hz, 1H), 6.21 (d- J = 8.8 Hz, 1H), 4.-0- |
| | | | 4.88 (m, 2H), 4.-0-4.76 (m, 2H), 4.-9- |
| | | | 4.50 (m, 1H), 4.40-4.30 (m, -H), 4.18 |
| | | | (s, 2H), 3.-9-3.24 (m, 2H), 3.19-3.15 |
| | | | (m, 2H), 2.50 (s, 3H), 1.04 (t, J = 7.1 |
| | | | Hz, 3H) |
| 156 | I-15 | 514.2 | (400 MHz, METHANOL-d4) δ 8.11 (s, |
| | | | 1H), 7.88 (s, -H), 7.79 (s, 1H), 6.99- |
| | | | 6.91 (m, 2H), 6.80 (d- J = 8.8 Hz, 1H), |
| | | | 4.20-4.17 (m, 4H), 4.03 (br dd, J = -.2, |
| | | | 11.2 Hz, 2H), 3.-3-3.85 (m, 1H), 3.-9- |
| | | | 3.52 (m, 2H), 3.29-3.25 (m, -H), 2.50 |
| | | | (s, 3H), 1.-7-1.75 (m, 2H), 1.71-1.66 |
| | | | (m, 2H), 1.05 (t, J = 7.2 Hz, 3H) |

### Synthesis of (S)-1-((5-bromo-3-fluoro-1H-indazol-7-yl)sulfonyl)-N-(1-methyl-2-oxo-1,2-dihydropyridin-4-yl)azetidine-2-carboxamide (Example 151)

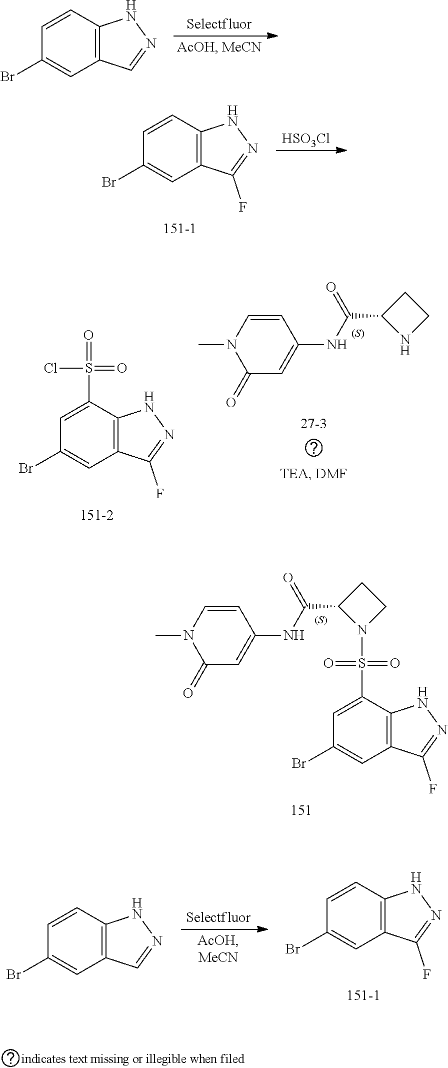

To a solution of 5-bromo-1H-indazole (1.00 g, 5.08 mmol) in a mixture of acetonitrile (10 mL) and glacial acetic acid (1 mL) was added with 1-(chloromethyl)-4-fluoro-1,4-diazoniabi cyclo[2.2.2]octane; ditetrafluoroborate (2.70 g, 7.61 mmol). The mixture was stirred at 80° C. for 4 hours under nitrogen atmosphere then poured into water (30 mL) and extracted with ethyl acetate (30 mL*3). The organic layers were dried over anhydrous sodium sulfate, filtered and the filtrate was concentrated under reduced pressure. The residue was purified by column chromatography (SiO₂, petroleum ether/ethyl acetate=20/1˜10/1) to afford 5-bromo-3-fluoro-1H-indazole (151-1) (0.600 g, 55.0% yield) as a yellow solid.

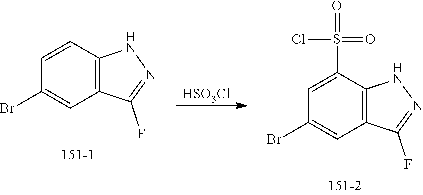

The mixture of 151-1 (0.200 g, 0.930 mmol) in chlorosulfonic acid (45.1 mmol, 3.00 mL) was stirred at 80° C. for 12 hours. The reaction mixture was poured into ice water (10 mL) and extracted with ethyl acetate (10 mL*3). The combined organic layers were dried over anhydrous sodium sulfate, filtered, and the filtrate was concentrated under reduced pressure. The residue was purified by column chromatography (SiO₂, petroleum ether/ethyl acetate=20/1˜10/1) to afford 5-bromo-3-fluoro-1H-indazole-7-sulfonyl chloride (151-2) (0.250 g, 85.7% yield) as a yellow solid.

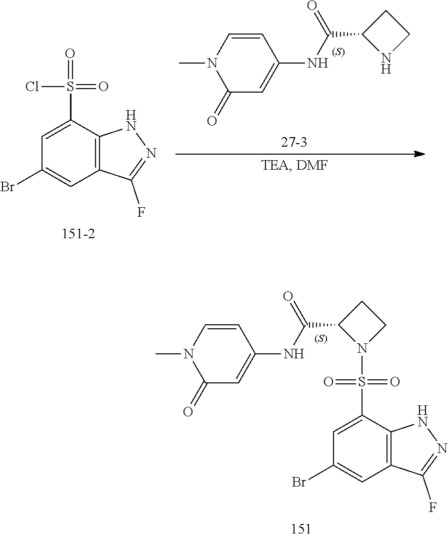

To a solution of 27-3 (0.210 g, 0.650 mmol) in tetrahydrofuran (5 mL) was added triethylamine (1.95 mmol, 0.270 mL) at 0° C. followed by a solution of 151-2 (0.200 g, 0.650 mmol) in tetrahydrofuran (5 mL) added drop wise at 0° C. After addition, the mixture was stirred at 0° C. for 1 hour. The reaction mixture was poured into ice water (10 mL) and extracted with ethyl acetate (10 mL*3). The combined organic layers were dried over anhydrous sodium sulfate, filtered, and the filtrate was concentrated under reduced pressure. The residue was purified by reversed phase flash (0.1% TFA/MeCN condition) and followed by prep-HPLC (column: Waters Xbridge 150*25 mm*5 um; mobile phase: [water (10 mM NH₄HCO₃)-ACN]; B %: 24%-54%, 9 min) and lyophilized to afford (S)-1-((5-bromo-3-fluoro-1H-indazol-7-yl)sulfonyl)-N-(1-m238ydroxya-oxo-1,2-dihydro pyridin-4-yl)azetidine-2-carboxamide (151) (0.160 g, 0.330 mmol, 50.0% yield) as a white solid.

LCMS of 151: m/z 485.8 [M+H]⁺

¹H-NMR of 151: (DMSO-d₆, 400 MHz) δ 13.19 (s, 1H), 10.18 (s, 1H), 8.46 (s, 1H), 7.99 (s, 1H), 7.63 (d-J=7.6 Hz, 1H), 6.-5-6.72 (m, 1H), 6.-3-6.32 (m, 1H), 4.-5-4.76 (m, 1H), 3.87-3.78 (m, —H), 3.36 (s, 3H), 2.33-2.24 (m, 2H)

### Synthesis of (S)-1-((5-(difluoromethyl)-1H-indazol-7-yl)sulfonyl)-N-(1-methyl-2-oxo-1,2-dihydropyridin-4-yl)azetidine-2-carboxamide (Example 152)

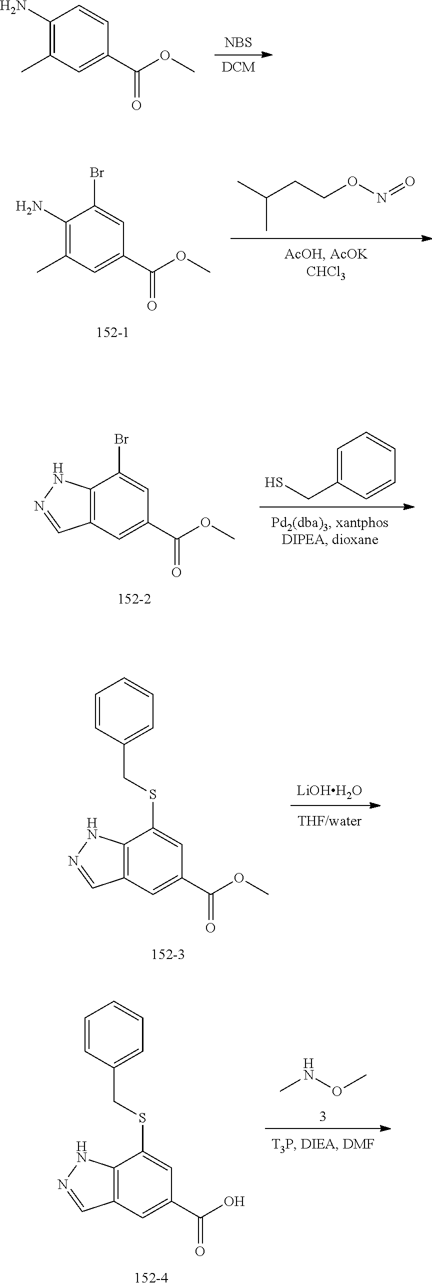

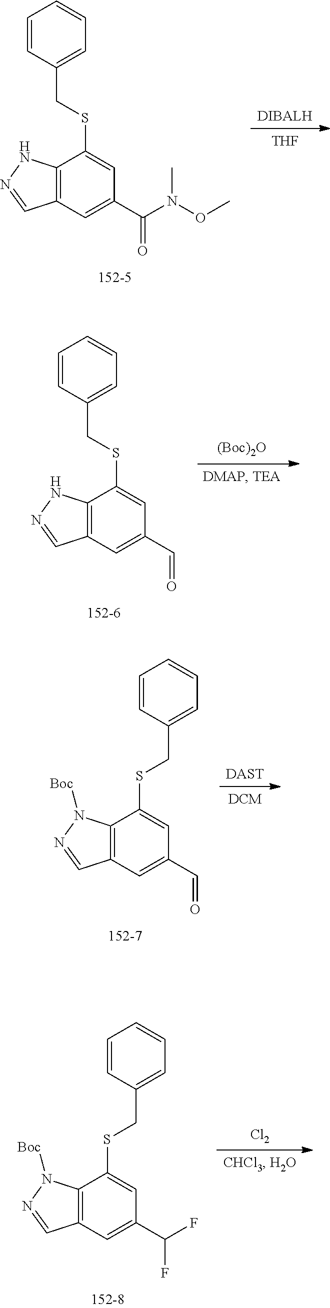

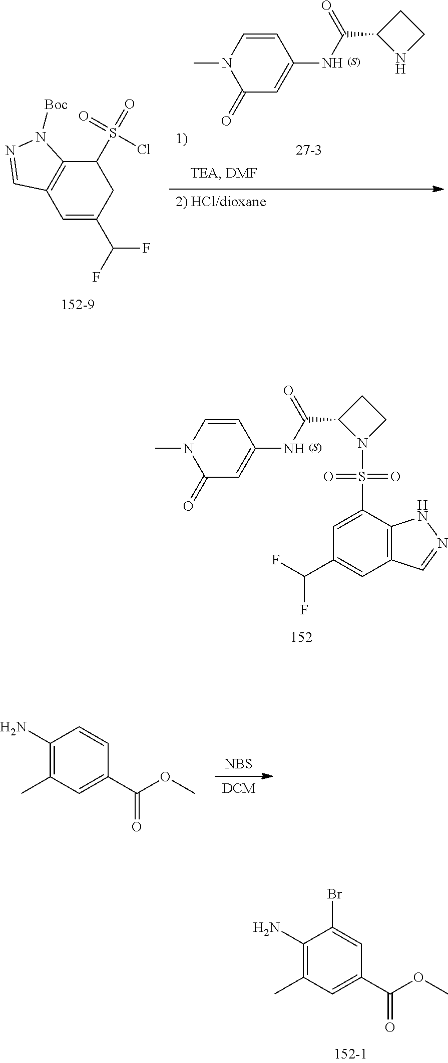

To a solution of methyl 4-amino-3-methylbenzoate (30.0 g, 181.6 mmol) in dichloromethane (360 mL) was added 1-bromopyrrolidine-2, 5-dione (48.4 g, 272 mmol). The mixture was stirred at 20° C. for 1 hour then diluted with water (500 mL) and extracted with dichloromethane (100 mL*2). The combined organic layers were washed with a saturated aqueous solution of sodium sulfite (500 mL) followed by brine (500 mL) then dried over anhydrous sodium sulfate, filtered, and the filtrate was concentrated under reduced pressure. The residue was triturated with petroleum ether (500 mL), then filtered and then collected cake dried to afford methyl 4-amino-3-bromo-5-methylbenzoate (152-1) (40.0 g, 90.2% yield) as a red solid. No further purification was performed.

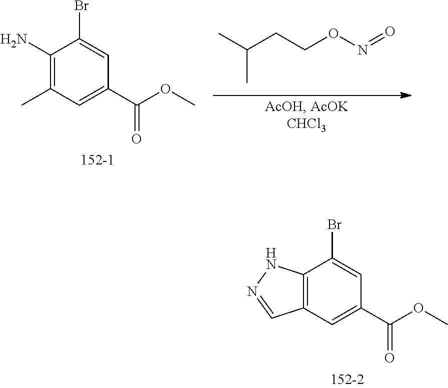

To a solution of 152-1 (43.0 g, 176 mmol) in chloroform (440 mL) at 0° C. was added potassium acetate (20.7 g, 311 mmol) followed by acetic acid (49.5 mL, 528.5 mmol). The mixture was stirred at 20° C. for 2 hours, then isopentyl nitrite (37.1 g, 317 mmol) was added at 60° C. and the reaction mixture was stirred at 60° C. for 12 hours. The mixture was quenched with 500 mL of cooled water and extracted with dichloromethane (500 mL*3). The combined organic layer was washed with brine (500 mL*2), dried over anhydrous sodium sulfate, filtered, and the filtrate was concentrated under reduced pressure to give a residue. The residue was diluted with a mixture of methanol and 6M hydrochloric acid (500 mL, v/v=1/1), the resulting mixture was stirred at 20° C. for 4 hours, then extracted with dichloromethane (500 mL*3). The combined organic layer was washed with a saturated aqueous solution of sodium bicarbonate (500 mL) followed by brine (500 mL) then dried over anhydrous sodium sulfate, filtered, and the filtrate was concentrated under reduced pressure to give a residue. The residue was triturated with a mixture of petroleum ether, ethyl acetate, and dichloromethane (80 mL:80 mL:80 mL), then filtered and the filter cake dried to afford methyl 7-bromo-1H-indazole-5-carboxylate (152-2) (36.0 g, 80% yield) as a yellow solid. No further purification was performed.

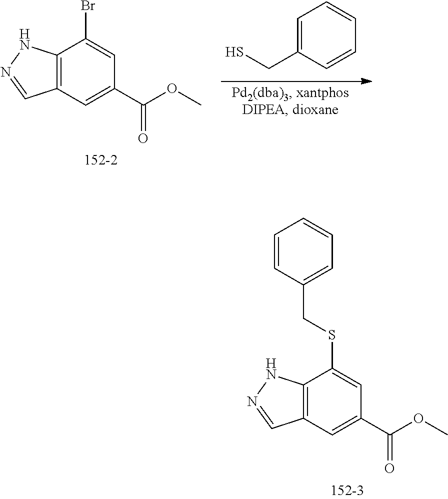 [SMILES: COC(=O)c1cc(Br)c2[nH]ncc2c1 ; SCc1ccccc1 ; COC(=O)c1cc(SCc2ccccc2)c2[nH]ncc2c1]

A mixture of 152-2 (15.0 g, 58.8 mmol), N, N-diisopropylethylamine (20.2 mL, 116.3 mmol), and phenylmethanethiol (10.4 mL, 88.3 mmol) in dioxane (150 mL) was degassed and purged with nitrogen three times. To this was added Xantphos (4.76 g, 8.23 mmol) and Pd₂(dba)₃ (3.77 g, 0.07 mmol) in one portion. The mixture was degassed and purged with nitrogen three times then stirred at 100° C. for 12 hours. After being cooled to room temperature, the mixture was combined with another batch (15 g scale), filtered, and washed with dichloromethane (250 mL*2). The filtrate was washed with 1 N hydrochloric acid (500 mL) followed by brine (500 mL) then dried over anhydrous sodium sulfate, filtered, and the filtrate was concentrated under reduced pressure. The residue was triturated with a mixture of petroleum ether:ethyl acetate: and dichloromethane (60 mL: 60 mL: 60 mL) then filtered and the filter cake dried to afford methyl 7-(benzylthio)-1H-indazole-5-carboxylate (152-3) (32.0 g, 91.2% yield) as a yellow solid. No further purification was performed.

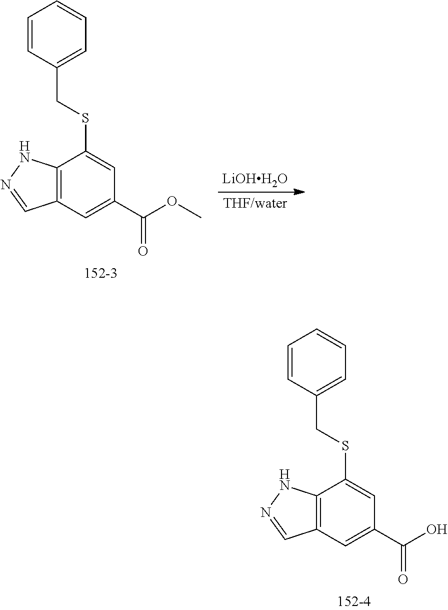 [SMILES: COC(=O)c1cc(SCc2ccccc2)c2[nH]ncc2c1 ; "LiOH·H2O" ; O=C(O)c1cc(SCc2ccccc2)c2[nH]ncc2c1]

To a mixture of 152-3 (1.90 g, 6.37 mmol) in a mixture of tetrahydrofuran (20 mL) and water (5 mL) was added lithium hydroxide monohydrate (0.801 g, 19.1 mmol). The mixture was stirred at 60° C. for 16 hours then the reaction mixture was concentrated under reduced pressure to remove tetrahydrofuran. The aqueous phase was adjusted pH to 4 with 1 N hydrochloric acid causing a solid to precipitate out. The mixture was filtered, and the filter cake was dried under reduced pressure to afford 7-(benzylthio)-1H-indazole-5-carboxylic acid (152-4) (1.20 g, 4.22 mmol, 66.2% yield) as a yellow solid. No further purification was performed.

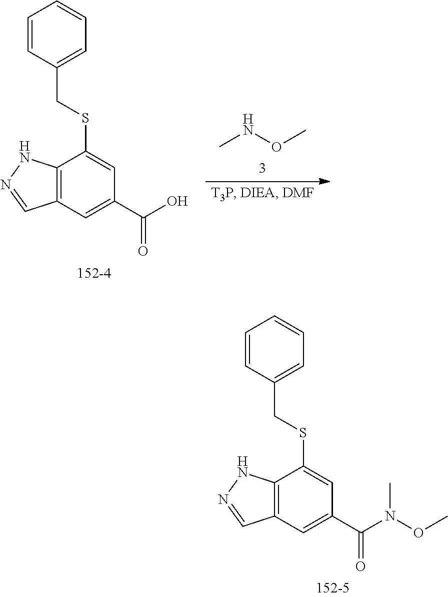

To a mixture of 152-4 (1.20 g, 4.22 mmol) and N,O-dimethylhydroxylamine hydrochloride (0.617 g, 6.33 mmol), and diisopropylethylamine (1.64 g, 12.6 mmol) in N, N-dimethylformamide (20 mL) was added T₃P (4.03 g, 6.33 mmol, 3.77 mL, 50% w/w in ethyl acetate) drop wise. The mixture was stirred at 25° C. for 2 hours then partitioned between water (50 mL) and ethyl acetate (200 mL). The organic phase was separated and washed with brine (30 mL*3) then dried over anhydrous sodium sulfate, filtered, and the filtrate was concentrated under reduced pressure. The residue was purified by silica gel chromatography (column height: 250 mm, diameter: 100 mm, 100-200 mesh silica gel, petroleum ether/ethyl acetate=100/1, 1/1) to afford 7-(benzylthio)-N-methoxy-N-methyl-1H-indazole-5-carboxamide (152-5) (1.10 g, 79.1% yield) as a yellow solid. No further purification was performed.

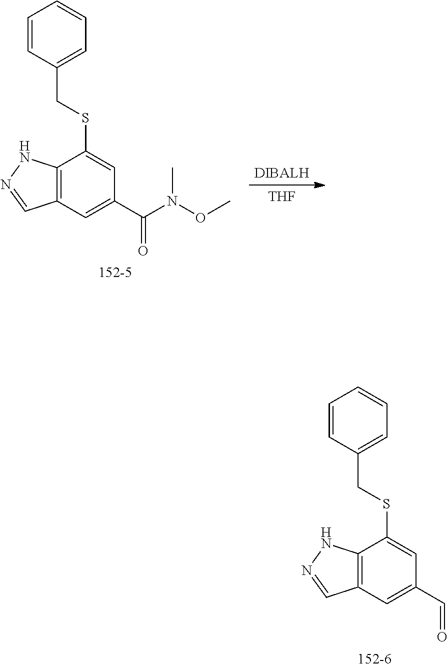 [SMILES: CON(C)C(=O)c1cc(SCc2ccccc2)c2[nH]ncc2c1 ; O=Cc1cc(SCc2ccccc2)c2[nH]ncc2c1 ; "THF"]

To a mixture of 152-5 (0.900 g, 2.75 mmol) in tetrahydrofuran (10 mL) was added DIBAL-H (1 M, 13.7 mL, 13.7 mmol) drop wise at −60° C. under nitrogen. The mixture was stirred at −60° C. for 1 hour then poured into a saturated aqueous solution of ammonium chloride (50 mL) and stirred for 5 minutes. The aqueous phase was extracted with ethyl acetate (100 mL*3). The combined organic phase was washed with brine (50 mL*3), dried over anhydrous sodium sulfate, filtered, and the filtrate was concentrated under reduced pressure to afford 7-(benzylthio)-1H-indazole-5-carbaldehyde (152-6) (0.600 g, 26.8% yield) as a yellow solid. No further purification was performed.

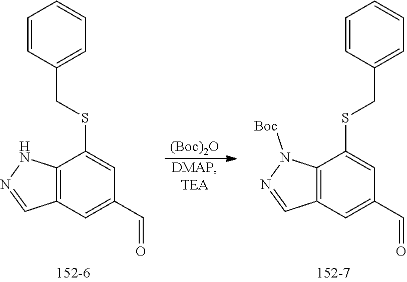 [SMILES: "(Boc)2O" ; O=Cc1cc(SCc2ccccc2)c2[nH]ncc2c1 ; "DMAP, TEA" ; CC(C)(C)OC(=O)n1ncc2cc(C=O)cc(SCc3ccccc3)c21]

To a mixture of 152-6 (0.600 g, 0.737 mmol)m triethylamine (0.112 g, 1.11 mmol) and Boc₂O (0.241 g, 1.11 mmol) in acetonitrile (10 mL) was added DMAP (9.01 mg, 0.073 mmol) under a nitrogen atmosphere. The mixture was stirred at 25° C. for 2 hours and the mixture was poured into ice-water (w/w=1/1) (20 mL) and stirred for 5 minutes. The aqueous phase was extracted with ethyl acetate (100 mL*3). The combined organic phase was washed with brine (50 mL*2), dried over anhydrous sodium sulfate, filtered, and the filtrate was concentrated under reduced pressure. The residue was purified by silica gel chromatography (column height: 250 mm, diameter: 100 mm, 100-200 mesh silica gel, petroleum ether/ethyl acetate=100/1, 5/1) to afford tert-butyl 7-(benzylthio)-5-formyl-1H-indazole-1-carboxylate (152-7) (0.200 g, 53.6% yield) as a yellow solid.

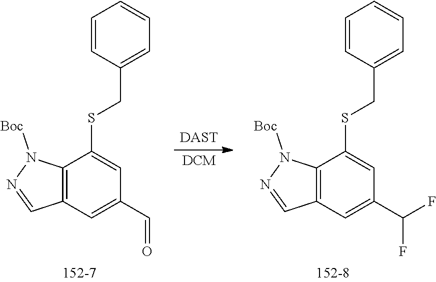

To a mixture of 152-7 (0.200 g, 0.395 mmol) in dichloromethane (10 mL) was added DAST (0.510 g, 3.17 mmol) drop wise at 0° C. under nitrogen atmosphere. The mixture was stirred at 25° C. for 12 hours. The mixture was poured into ice-water (w/w=1/1, 50 mL) and stirred for 5 minutes. The aqueous phase was extracted with dichloromethane (200 mL*3). The combined organic phase was washed with a saturated aqueous solution of sodium bicarbonate (30 mL*3) then dried over anhydrous sodium sulfate, filtered, and the filtrated was concentrated under reduced pressure. The residue was purified by prep-TLC (SiO₂, petroleum ether/ethyl acetate=5/1) to afford tert-butyl 7-(benzylthio)-5-(difluoromethyl)-1H-indazole-1-carboxylate (152-8) (0.070 g, 44% yield) as a yellow solid.

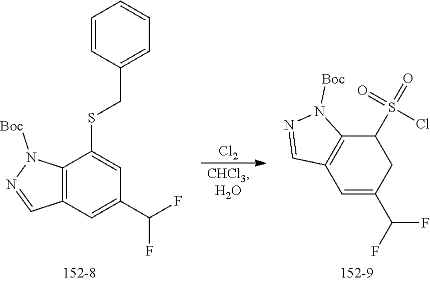

Chlorine gas was bubbled into a solution of 152-8 (0.070 g, 0.18 mmol) in a mixture of chloroform (4 mL) and water (2 mL) at 0° C. for 10 minutes. After excess chlorine was purged by nitrogen the mixture was poured into ice-water (w/w=1/1) (5 mL) and stirred for 5 minutes. The aqueous phase was extracted with dichloromethane (20 mL*3). The combined organic phase was washed with brine (10 mL*2), dried over anhydrous sodium sulfate, filtered, and the filtrate was concentrated under reduced pressure to afford tert-butyl 7-(chlorosulfonyl)-5-(difluoromethyl)-1H-indazole-1-carboxylate (152-9) (0.060 g, crude) as yellow gum. No further purification was performed.

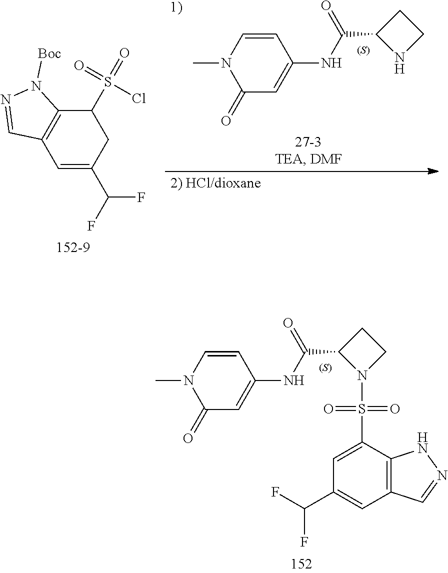

To a mixture of 27-3 (0.036 g, 0.11 mmol) and triethylamine (0.049 g, 0.49 mmol) in N, N-dimethylformamide (1 mL) was added 152-9 (0.060 g, 0.16 mmol) at 0° C. The mixture was stirred at 25° C. for 1 hour then poured into ice-water (w/w=1/1, 5 mL) and stirred for 5 minutes. The aqueous phase was extracted with ethyl acetate (20 mL*3) and the combined organic phase was washed with brine (20 mL*2), dried over anhydrous sodium sulfate, filtered, and the filtrate was concentrated under reduced pressure. The residue was taken up in dioxane (1 mL) and a solution of HCl in dioxane (4 M, 0.217 mL, 0.870 mmol) was added. The mixture was stirred at 25° C. for 1 hour, then concentrated under reduced pressure. The residue was purified by reversed-phase flash (0.1% NH₃·H₂O/MeCN condition) to afford (S)-1-((5-(difluoromethyl)-1H-indazol-7-yl)sulfonyl)-N-(1-methyl-2-oxo-1,2-dihydropyridin-4-yl)azetidine-2-carboxamide (152) (0.013 g, 77.3% yield) as a white solid.

LCMS of 152: m/z=437.9 [M+H]⁺

¹H NMR of 152: (CD₃OD, 400 MHz) δ 8.41-8.32 (m, 2H), 8.07 (s, 1H), 7.61 (d-J=7.6 Hz, 1H), 7.-3-6.85 (m, 2H), 6.-2-6.70 (m, 1H), 4.-3-4.91 (m, 1H), 4.-0-3.93 (m, 1H), 3.70-3.65 (m, —H), 3.53 (s, 3H), 2.45-2.40 (m, 2H)

### Synthesis of (S)—N-(3-chloro-4-(4-methylpiperazin-1-yl)phenyl)-1-((5-methyl-1H-indazol-7-yl)sulfonyl)azetidine-2-carboxamide (Example 153)

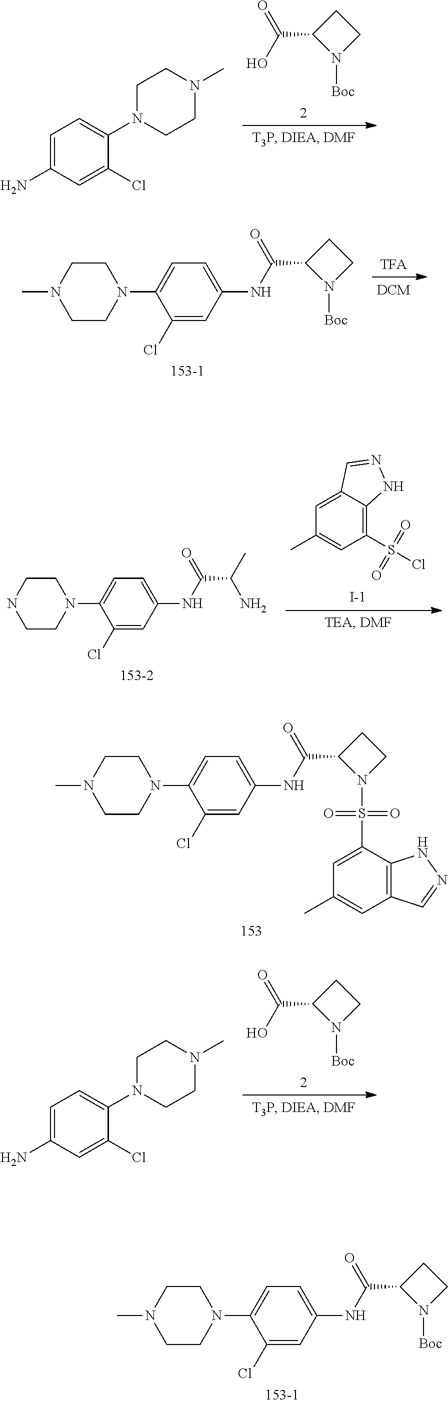

To a mixture of 3-chloro-4-(4-methylpiperazin-1-yl)aniline (0.200 g, 0.886 mmol), (S)-1-(tert-butoxycarbonyl)azetidine-2-carboxylic acid (0.180 g, 0.895 mmol) and diisopropylethylamine (0.650 mL, 3.73 mmol) in dimethyl formamide (3 mL) was added T₃P (0.800 mL, 1.35 mmol, 50% w/w in ethyl acetate) at 0° C. drop wise. The mixture was stirred at 0° C. for 1 hour then poured into ice water (50 mL) and extracted with ethyl acetate (20 mL*3). The combined organic layers were washed with brine (20 mL), dried over anhydrous sodium sulfate, filtered, and the filtrate was concentrated under reduced pressure to afford tert-butyl (S)-2-((3-chloro-4-(4-methylpiperazin-1-yl)phenyl)carbamoyl) azetidine-1-carboxylate (153-1) (0.330 g, 91% yield) as a yellow solid. No further purification was performed.

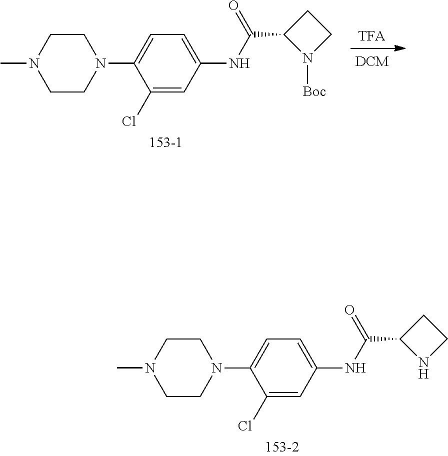 [SMILES: CN1CCN(c2ccc(NC(=O)[C@@H]3CCN3C(=O)OC(C)(C)C)cc2Cl)CC1 ; CN1CCN(c2ccc(NC(=O)[C@@H]3CCN3)cc2Cl)CC1]

To a solution of 153-1 (0.150 g, 0.367 mmol) in dichloromethane (2 mL) was added trifluoroacetic acid (1.00 mL, 13.5 mmol) at 0° C. The mixture was stirred at 0° C. for 2 hours then concentrated under reduced pressure to afford (S)—N-(3-chloro-4-(4-methylpiperazin-1-yl)phenyl)azetidine-2-carboxamide (153-2)trifluoroacetic acid salt (0.100 g, 64% yield) as a black oil which was used directly.

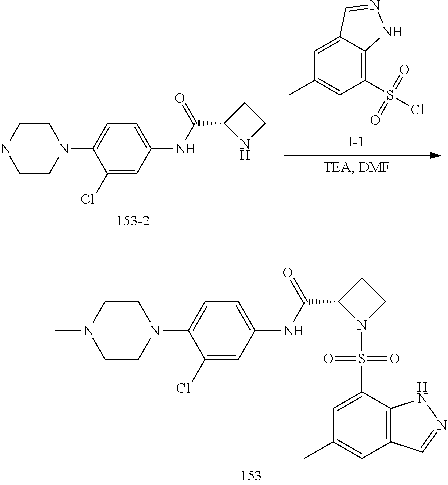

To a solution of 153-2 trifluoroacetic acid salt (0.050 g, 0.118 mmol) in dimethyl formamide (1 mL) was added triethylamine (0.11 mL, 0.787 mmol). Then I-1 (0.030 g, 0.127 mmol) was added at 0° C. The mixture was stirred at 0° C. for 1 hour then poured into water (10 mL) and extracted with ethyl acetate (10 mL*3). The combined organic layers were washed with brine (20 mL), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH₄HCO₃)-ACN]; B %: 32%-62%, 10 min) to afford (S)—N-(3-chloro-4-(4-methylpiperazin-1-yl)phenyl)-1-((5-methyl-1H-indazol-7-yl)sulfonyl)azetidine-2-carboxamide (153) (0.052 g, 090% yield) as a white solid.

LCMS of 153: m/z 503.1 [M+H]⁺

¹H NMR of 153: (CDCl₃, 400 MHz) δ 11.10 (br, s, 1H), 8.72 (s, 1H), 8.17 (s, —H), 7.91 (s, 1H), 7.70-7.65 (m, 2H), 7.44 (dd, J=8.4, 2.4 Hz, 1H), 7.05 (d, J=8.4 Hz, 1H), 4.47 (dd, J=10.0, 8.0 Hz, 1H), 3.86 (dt, J=8.8, 4.4 Hz, 1H), 3.69 (q, J=8.8 Hz, 1H), 3.08 (s, 4H), 2.64 (s, —H), 2.58 (s, 3H), 2.49-2.41 (m, —H), 2.38 (s, 3H), 2.34-2.24 (m, 1H)

The following compound was synthesized in a similar manner as 153 using the reactant indicated.

| | | LCMS | |
|---|---|---|---|
| Example | Reactant | m/z | ¹H-NMR |
| 168 | 164-1 | 492.1 | (CD₃OD, 400 MHz) δ 8.16 (s, 1H), 7.94 |
| | | | (s, 1H), 7.78 (s, 1H), 7.71 (d, J = 2.4 |
| | | | Hz, 1H), 7.42 (dd, J = 2.4, 8.8 Hz, 1H), |
| | | | 7.07 (d- J = 8.8 Hz, 1H), 4.82-4.78 (m, |
| | | | 1H), 4.18 (t, J = 5.6 Hz, 2H), -.95-3.88 |
| | | | (m, 1H), 3.74-3.65 (m, 1H), 2.85 (t, J = |
| | | | 5.2 Hz, -H), 2.53 (s, 3H), 2.43-2.35 (m, |
| | | | 8H) |
| 192 | I-48 | 472.1 | (CD₃OD, 400 MH-) δ 8.16 (s, 1H), |
| | | | 7.94-7.93 (m, 1H), 7.78 (d, J = 0.8 Hz, |
| | | | 1H), 7.37 (dd, J₁ = 2.4 Hz, J₂ = 8.8 Hz, |
| | | | 1H), 7.30 (d, J = 2.0 Hz, 1H), 6.88 (d- |
| | | | J = 8.8 Hz, 1H), 4.82-4.78 (m, 1H), 4.12 |
| | | | (t- J = 5.2 Hz, 2H), 3.-4-3.88 (m, 1H), |
| | | | 3.72-3.67 (m, 1H), 2.82 (t, J = 5.2 Hz, |
| | | | 2H), 2.53 (s, -H), 2.38 (s, 6H), 2.37- |
| | | | 2.35 (m, 2H), 2.23 (s, 3H) |

The following compound was synthesized in a similar manner using 24-1 in replace of I-3 and the reactant indicated.

| | | LCMS | |
|---|---|---|---|
| Example | Reactant | m/z | ¹H-NMR |
| 161 | I-15 | 532.1 | (DMSO-d₆, 400 MHz) δ 13.89 (br. s, |
| | | | 1H), -9.97 (br. s, 1H), 8.38-8.24 (m, |
| | | | 2H), 7.79 (d, J = 1.6 Hz, 1H), 7.07 (d, |
| | | | J = 2.4 Hz, 1H), 6.98 (d, J = 8.4 Hz, |
| | | | 1H), 6.84 (d, J = 9.2 Hz, 1H), 4.87 (t- |
| | | | J = 8.0 Hz, 1H), 4.-1-4.06 (m, 2H), 3.- |
| | | | 7-3.76 (m, 4H), 3.-7-3.58 (m, 1H), 3.- |
| | | | 0-3.35 (m, 3H), 3.-1-3.14 (m, 2H), 2.- |
| | | | 6-2.25 (m, 2H), 1.-2-1.62 (m, 2H), |
| | | | 1.60-1.52 (m, 2H) |

The following compound was synthesized in a similar manner using I-40 in replace of I-3 and the reactant indicated.

| | | LCMS | |
|---|---|---|---|
| Example | Reactant | m/z | ¹H-NMR |
| 162 | I-15 | 548.1 | (DMSO-d6, 400 MHz): δ 14.15 (br. s, |
| | | | 1H), -0.03 (br. s, 1H), 8.47-8.45 (m, |
| | | | -H), 7.98 (s, 1H), 7.-0-7.00 (m, 3H), |
| | | | 6.-0-6.80 (m, 1H), 4.-5-4.97 (m, 1H), |
| | | | 4.-0-4.10 (m, 2H), 4.-0-3.80 (m, 4H), |
| | | | 3.-0-3.50 (m, 1H), 3.-0-3.30 (m, 2H), |
| | | | 3.-0-3.10 (m, 2H), 2.-0-2.20 (m, 2H), |
| | | | 1.8--1.60 (m, 2H), 1.60-1.40 (m, 2H) |

The following compounds were synthesized in a similar manner using I-43 in replace of I-3 and the reactants indicated.

| | | LCMS | |
|---|---|---|---|
| Example | Reactant | m/z | ¹H-NMR |
| 167 | I-15 | 550.1 | (DMSO-d6, 400 MHz) δ 13.48 (s, 1H), |
| | | | 9.93 (s, 1H), 8.40-8.18 (m, 1H), 7.99- |
| | | | 7.77 (m, 1H), 7.24-6.88 (m, 2H), 6.85 |
| | | | (d, J = 8.8 Hz, 1H), 4.87 (t, J = 8.0 Hz, |
| | | | 1H), 4.21-4.12 (m, 2H), 4.00-3.80 (m, |
| | | | 4H), 3.76-3.62 (m, 1H), 3.49-3.41 (m, |
| | | | 2H), 3.25-3.14 (m, 2H), 2.35-2.29 (m, |
| | | | 2H), 1.78-1.54 (m, 4H) |
| 177 | I-25 | 536.2 | (DMSO-d6, 400 MHz): δ 14.22 (br. s, |
| | | | 1H), 10.01 (s, 1H), 8.47 (s, 2H), 8.01 |
| | | | (s, 1H), 7.26 (t, J = 55.6 Hz, 1H), 7.09 |
| | | | (d- J = 2.4 Hz, 1H), 7.01-6.96 (m, 1H), |
| | | | 6.92 (dd, J1 = 2.4 Hz, J2 = 8.8 Hz, 1H), |
| | | | 4.96 (t, J = 8.4 Hz, 1H), 4.78 (t, J = 5.6 |
| | | | Hz, 1H), 4.17 (t- J = 8.0 Hz, 2H), 3.-3- |
| | | | 3.81 (m, 1H), 3.67-3.57 (m, 1H), 3.48 |
| | | | (d- J = 5.6 Hz, 2H), 3.-1-3.26 (m, 2H), |
| | | | 2.40-2.29 (m, 2H), 1.24 (s, 6H) |

### Synthesis of 2-((N,5-dimethyl-1H-indazole)-7-sulfonamido)-N-(4,5,6,7-tetrahydrothiazolo[5,4-b]250yclopro-2-yl)acetamide (Example 154)

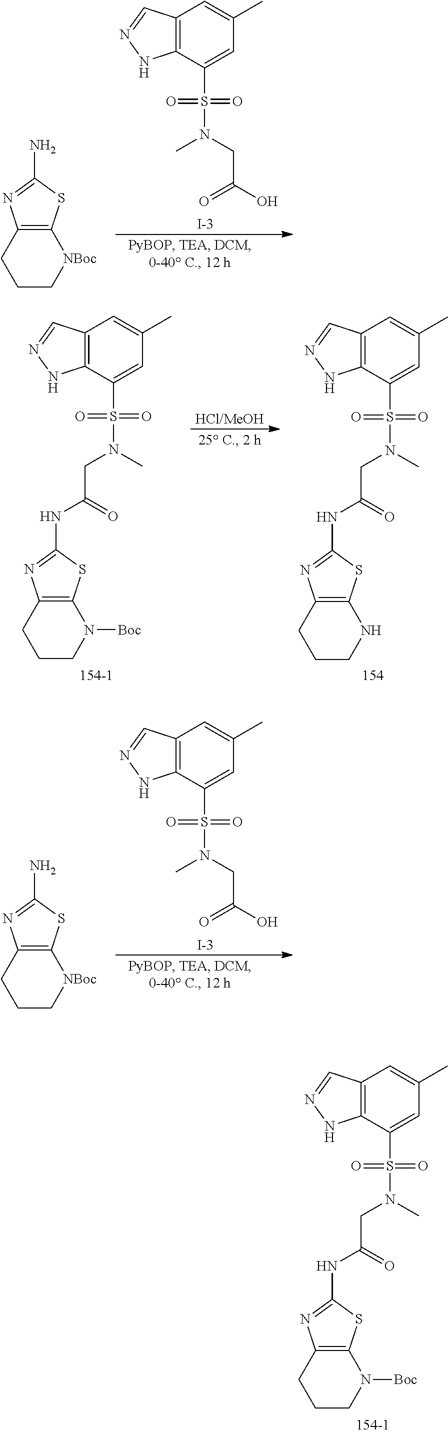

To a solution of tert-butyl 2-amino-6,7-dihydro-5H-thiazolo[5,4-b]pyridine-4-carboxylate (0.300 g, 1.17 mmol), I-3 (0.222 g, 0.783 mmol) and PYBOP (0.815 g, 1.57 mmol) in DCM (10 mL) was added TEA (0.545 mL, 3.92 mmol) at 0° C. under N2. The mixture was stirred at 40° C. for 12 hr under N₂ then concentrated in vacuum. The residue was purified by flash silica gel chromatography to afford tert-butyl 2-[[2-[methyl-[(5-methyl-1H-indazol-7-yl)sulfonyl]amino]acetyl]amino]-6,7-dihydro-5H-thiazolo[5,4-b]pyridine-4-carboxylate (154-1) (0.4 g, 640.16 umol, 81.73% yield, 83.32% purity) was obtained as a yellow oil.

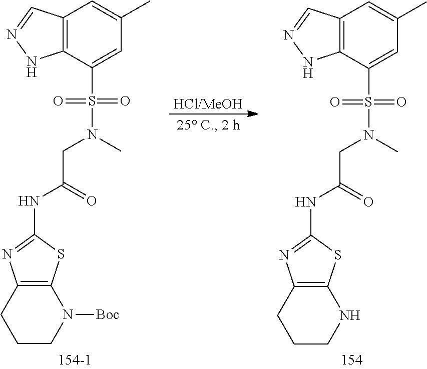 [SMILES: Cc1cc(S(=O)(=O)N(C)CC(=O)Nc2nc3c(s2)N(C(=O)OC(C)(C)C)CCC3)c2[nH]ncc2c1 ; "HCl/MeOH" ; Cc1cc(S(=O)(=O)N(C)CC(=O)Nc2nc3c(s2)NCCC3)c2[nH]ncc2c1]

A mixture of 154-1 (0.40 g, 0.77 mol, 1 eq) in HCl/MeOH (10 mL, 4M) was stirred at 25° C. for 2 hr. The reaction mixture was concentrated under vacuum to give a crude product (0.35 g). The crude product (0.15 g) was purified by prep-HPLC (HCl condition) to give 2-[methyl-[(5-methyl-1H-indazol-7-yl) sulfonyl]amino]-N-(4,5,6,7-tetrahydrothiazolo[5,4-b]251yclopro-2-yl)acetamide (154) (0.026 g, 7.2% yield) as a white solid.

LCMS for 154: m/z 421.0 [M+H]⁺

¹H NMR for 154: (400 MHz, METHANOL-d4) δ 8.14 (s, 1H), 7.91 (s, 1H), 7.75 (s, 1H), 4.30 (s, 2H), 3.65-3.52 (m, 2H), 2.96 (s, 3H), 2.81 (br t, J=6.2 Hz, 2H), 2.54 (s, 3H), 2.21 (td, J=5.3, 10.6 Hz, 2H)

### Synthesis of N-(3-chloro-4-(2-(dimethylamino)ethoxy)phenyl)-2-((N,5-dimethyl-1H-indazole)-7-sulfonamido)acetamide (Example 164)

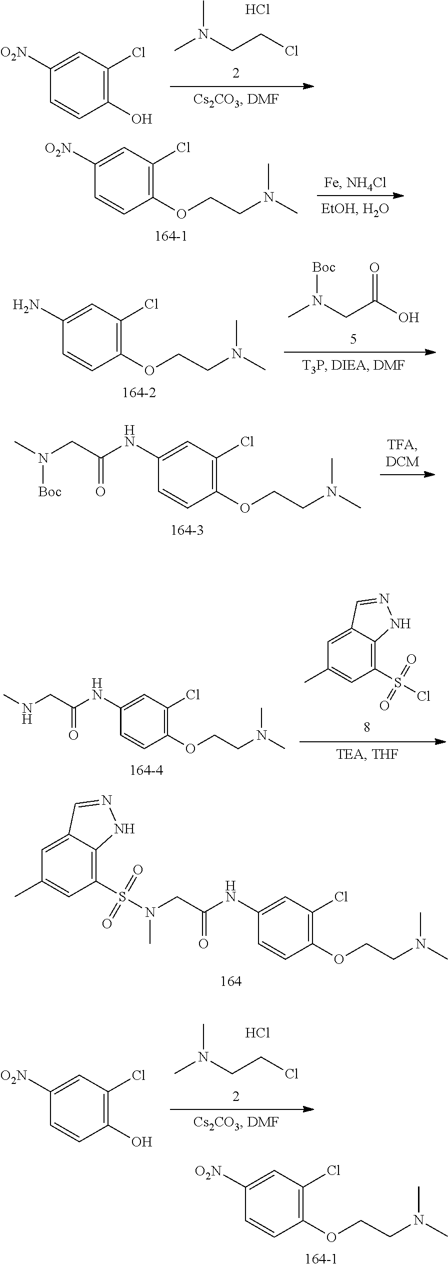

To a solution of 2-chloro-4-nitrophenol (3.47 g, 20.0 mmol) in N, N-dimethyl formamide (40 mL) was added 2-chloro-N, N-dimethylethan-1-amine hydrochloride (2) as the hydrochloric salt (2.88 g, 20.0 mmol) followed by cesium carbonate (9.77 g, 30.0 mmol). The mixture was stirred at 100° C. for 2.5 hours then diluted with water (100 mL) and extracted with ethyl acetate (50 mL*3). The extracts were washed by brine (100 mL*3), dried over anhydrous sodium sulfate, filtered, and the filtrate was concentrated under reduced pressure. The crude product was purified by reversed-phase flash (0.1% TFA/MeCN condition) to afford 2-(2-chloro-4-nitrophenoxy)-N, N-dimethylethan-1-amine (164-1) (0.690 g, 13.9% yield) as a yellow solid.

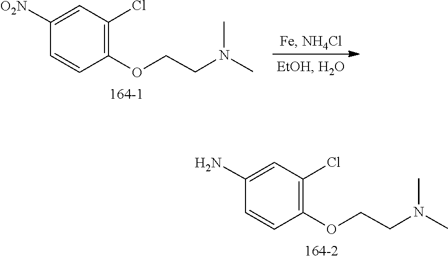 [SMILES: CN(C)CCOc1ccc([N+](=O)[O-])cc1Cl ; CN(C)CCOc1ccc(N)cc1Cl]

To a solution of 164-1 (0.200 g, 0.817 mmol) in a mixture of water (1 mL) and ethanol (2 mL) was added ammonium chloride (0.350 g, 6.54 mmol) and iron powder (0.365 g, 6.54 mmol). Then the mixture was stirred at 90° C. for 2.5 hours. The mixture was diluted with methanol (50 mL), filtered, and the filtrate was concentrated under reduced pressure. The residue was dissolved in water (50 mL, pH=8) and extracted with ethyl acetate (30 mL*3). The extracts were washed by brine (50 mL*3), dried over anhydrous sodium sulfate, filtered, and the filtrate was concentrated under reduced pressure to afford 3-chloro-4-(2-(dimethylamino)ethoxy)aniline (164-2) (0.100 g, 54.1% yield) as brown oil. No further purification was performed.

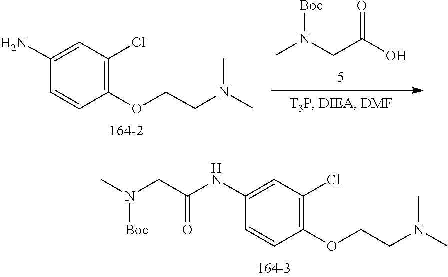

To a solution of 164-2 (0.100 g, 0.466 mmol) in N, N-dimethylformamide (1 mL) at 0° C. was added N, N-diisopropylethylamine (0.300 mL, 1.72 mmol) and T₃P (0.400 mL, 0.673 mmol, 50% w/w in ethyl acetate) drop wise. The mixture was stirred at 25° C. for 1 hour then diluted with a saturated aqueous solution of sodium bicarbonate (50 mL) and extracted with ethyl acetate (30 mL*3). The extracts were washed by brine (50 mL*3), dried over anhydrous sodium sulfate, filtered, and the filtrate was concentrated under reduced pressure to afford tert-butyl (2-((3-chloro-4-(2-(dimethylamino)ethoxy)phenyl)amino)-2-oxoethyl)(methyl)carbamate (164-3) (0.175 g, 94.7% yield) as brown oil. No further purification was performed.

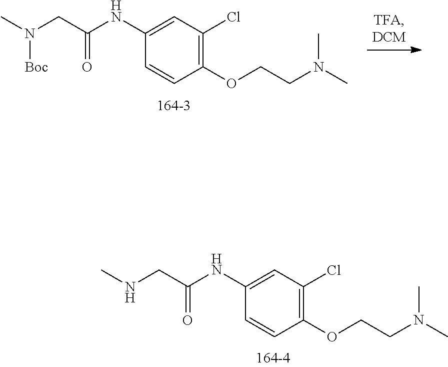

To a solution of 164-3 (0.175 g, 0.454 mmol) in dichloromethane (2 mL) at 0° C. was added trifluoroacetic acid (1 mL, 13.5 mmol) drop wise. The mixture was stirred at 25° C. for 1 hour then concentrated under reduced pressure to afford N-(3-chloro-4-(2-(dimethylamino)ethoxy)phenyl)-2-(methylamino)acetamide (164-4) as the di-trifluoroacetic salt (0.233 g, 94.7% yield) as brown oil. No further purification was performed.

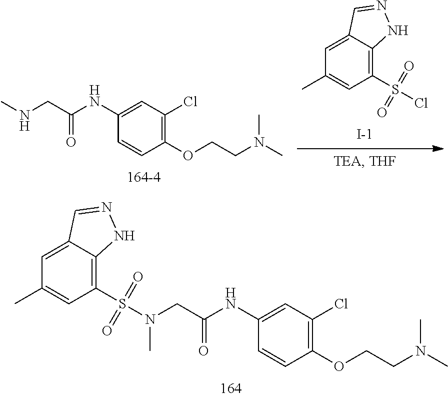

To a solution of 164-4 as the di-trifluoroacetic salt (0.116 g, 0.226 mmol) in tetrahydrofuran (1 mL) at 0° C. was added triethylamine (0.100 mL, 0.718 mmol) followed by a solution of I-1 (0.052 g, 0.226 mmol) in tetrahydrofuran (1 mL). The mixture was stirred at 0° C. for 1 hour then mixture was diluted with water (10 mL) and extracted with ethyl acetate (10 mL*3). The combined organic phase was washed by brine (20 ml*3), dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (10 mM NH₄HCO₃)—CAN]; B %: 25%-55%, 8 min) to afford N-(3-chloro-4-(2-(dimethylamino)ethoxy)phenyl)-2-((N,5-dimethyl-1H-indazole)-7-sulfonamido)acetamide (164) (0.044 g, 36.% yield) as a white solid.

LCMS of 164: m/z 480.1 [M+H]⁺

¹H-NMR of 164: (400 MHz, CDCl₃) δ 11.48 (br. S, 1H), 8.34 (s, 1H), 8.14 (s, 1H), 7.85 (s, 1H), 7.66 (d, J=2.4 Hz, 1H), 7.65 (d, J=0.8 Hz, 1H), 7.39 (dd, J=8.8, 2.4 Hz, 1H), 6.89 (d, J=8.8 Hz, 1H), 4.19 (t, J=5.6 Hz, 2H), 3.91 (s, 2H), 2.94-2.90 (m, 5H), 2.56 (s, 3H), 2.48 (s, 6H)

### Synthesis of N-1-((5-methyl-1H-indazol-7-yl)sulfonyl)-N-(4-(oxetan-3-yl)-3,4-dihydro-2H-benzo[b][1,4]oxazin-7-yl)piperidine-3-carboxamide (Example 170)

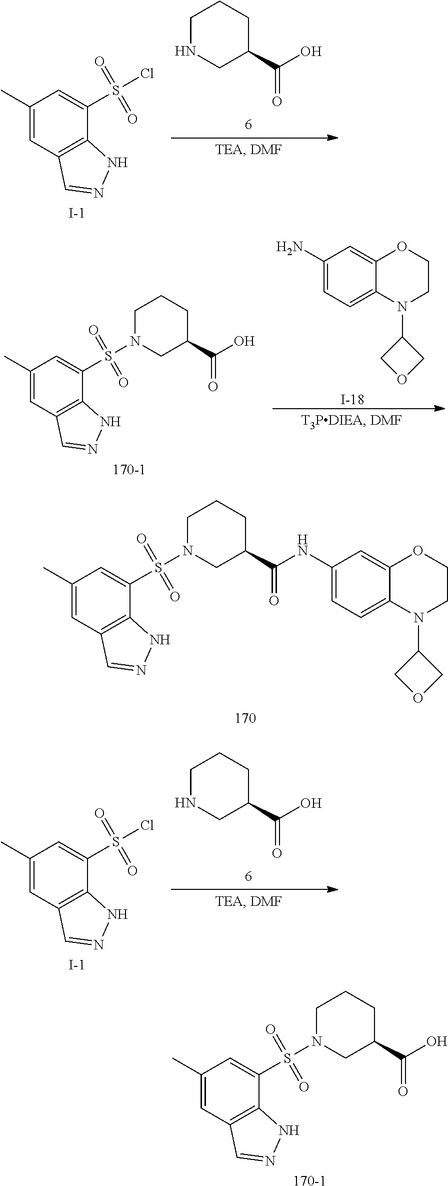

To a mixture of (R)-piperidine-3-carboxylic acid (0.100 g, 0.774 mmol) and triethylamine (0.400 mL, 2.87 mmol) in N, N-dimethylformamide (1 mL), at 0° C., was added I-1 (0.179 g, 0.776 mmol). The mixture was stirred at 0° C. for 1 hour then a saturated aqueous solution of potassium carbonate (1 mL) was added. The mixture was stirred at 25° C. for 11 hours then diluted with a saturated aqueous solution of potassium carbonate (50 mL) and washed with ethyl acetate (50 mL*3). The aqueous phase was adjusted to pH=4 with 1M hydrochloric acid and extracted with ethyl acetate (70 mL*2). The combined organic phase was washed with brine (100 mL*3), dried over anhydrous sodium sulfate, filtered, and the filtrate was concentrated under reduced pressure to afford®-1-((5-methyl-1H-indazol-7-yl) sulfonyl)piperidine-3-carboxylic acid (170-1) (0.110 g, 35.5% yield) as a white solid. No further purification was performed.

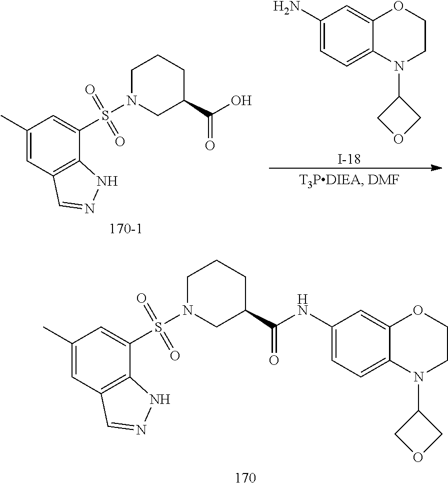

To a mixture of I-18 (0.028 g, 0.14 mmol) and 170-1 (0.055 g, 0.14 mmol) in N, N-dimethylformamide (0.5 mL) at 0° C. was added N, N-diisopropylethylamine (0.100 mL, 0.574 mmol) and T₃P (0.120 mL, 0.202 mmol, 50% w/w in ethyl acetate). The mixture was stirred at 0° C. for 1 hour then diluted with water (10 mL) and extracted with ethyl acetate (10 mL*2). The extracts were washed by brine (20 mL*3), dried over anhydrous sodium sulfate, filtered, and the filtrate was concentrated under reduced pressure at 25° C. The residue was purified by prep-HPLC (column: Phenomenex Gemini-NX Cis 75*30 mm*3 um; mobile phase: [water (0.05% ammonia hydroxide v/v)-CAN]; B %: 22%-52%, 7 min) to afford CAN-1-((5-methyl-1H-indazol-7-yl) sulfonyl)-N-(4-(oxetan-3-yl)-3,4-dihydro-2H-benzo[b][1,4]oxazin-7-yl) piperidine-3-carboxamide (170) (0.027 g, 27% yield) as a white solid.

LCMS of 170: m/z 512.2 [M+H]⁺

¹H-NMR of 170: (400 MHz, DMSO-d₆) δ 13.12 (br. S, 1H), 9.76 (s, 1H), 8.20 (s, 1H), 7.92 (s, 1H), 7.56 (s, 1H), 7.05 (d, J=2.4 Hz, 1H), 6.90 (dd, J=8.4, 2.4 Hz, 1H), 6.22 (d, J=8.8 Hz, 1H), 4.74 (t, J=6.4 Hz, 2H), 4.64 (t, J=6.4 Hz, 2H), 4.50-4.44 (m, 1H), 4.29-4.24 (m, 2H), 4.00-3.93 (m, 1H), 3.83-3.77 (m, 1H), 3.15-3.11 (m, 2H), 2.52-2.52 (m, 1H), 2.48 (s, 3H), 2.30-2.15 (m, 2H), 1.81-1.71 (m, 2H), 1.52-1.42 (m, 1H), 1.36-1.28 (m, 1H)

### Synthesis of 2-((5-(difluoromethyl)-N-methyl-1H-indazole)-7-sulfonamido)-N-(4-(tetrahydro-2H-pyran-4-yl)-3,4-dihydro-2H-benzo[b][1,4]oxazin-7-yl)acetamide (Example 171)

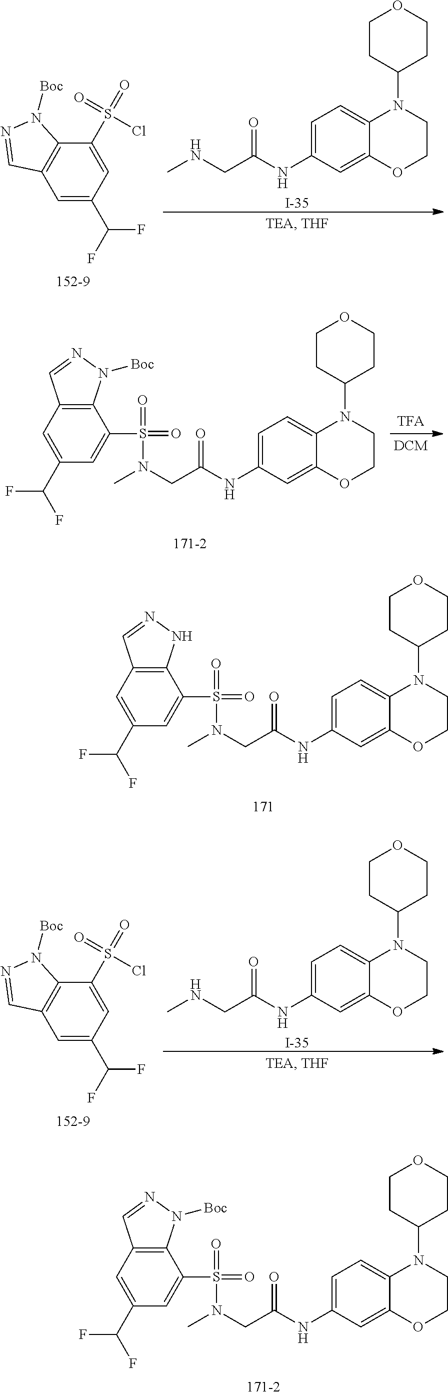

To a mixture of I-35 (0.200 g, 0.477 mmol) and triethylamine (0.200 mL, 1.44 mmol) in tetrahydrofuran (2 mL) at 0° C. was added a solution of 152-9 (0.540 g, 0.736 mmol) in tetrahydrofuran (2 mL). The mixture was stirred at 25° C. for 0.5 hour then diluted with water (50 mL) and extracted with ethyl acetate (50 mL*3). The combined organic layers were washed with brine (100 mL*3), dried over anhydrous sodium sulfate, filtered, and the filtrate was concentrated under reduced pressure to afford tert-butyl 5-(difluoromethyl)-7-(N-methyl-N-(2-oxo-2-((4-(tetrahydro-2H-pyran-4-yl)-3,4-dihydro-2H-benzo[b][1,4]oxazin-7-yl)amino)ethyl)sulfamoyl)-1H-indazole-1-carboxylate (171-2) (0.400 g, crude) as brown oil. No further purification was performed.

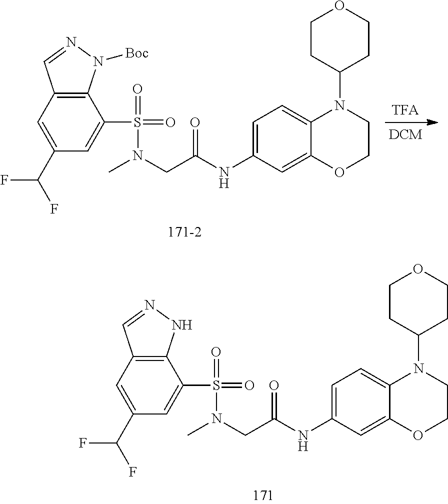 [SMILES: CN(CC(=O)Nc1ccc2c(c1)OCCN2C1CCOCC1)S(=O)(=O)c1cc(C(F)F)cc2cnn(C(=O)OC(C)(C)C)c12 ; CN(CC(=O)Nc1ccc2c(c1)OCCN2C1CCOCC1)S(=O)(=O)c1cc(C(F)F)cc2cn[nH]c12]

To a solution of 171-2 (0.090 g, 0.14 mmol) in dichloromethane (2 mL) at 0° C. was added trifluoroacetic acid (1.00 mL, 13.5 mmol). The mixture was stirred at 25° C. for 1 hour. The mixture was diluted with a saturated aqueous solution of sodium bicarbonate (50 mL) and extracted with ethyl acetate (50 mL*3). The combined organic layers were washed with brine (100 mL*3), dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (0.05% ammonia hydroxide v/v)-CAN]; B %: 25%-55%, 7 min) to afford 2-((5-(difluoromethyl)-N-methyl-1H-indazole)-7-sulfonamido)-N-(4-(tetrahydro-2H-pyran-4-yl)-3,4-dihydro-2H-benzo[b][1,4]oxazin-7-yl)acetamide (171) (0.029 g, 0.053 mmol) as a gray solid.

LCMS of 171: m/z=536.2 [M+H]⁺

¹H-NMR of 171: (DMSO-d₆, 400 MHz) δ 13.71 (br. S, 1H), 9.77 (br. S, 1H), 8.42 (d, J=12.8 Hz, 2H), 7.94 (s, 1H), 7.22 (t, J=15.6 Hz, 1H), 6.93 (s, 1H), 6.91-6.83 (m, 1H), 6.82-6.75 (m, 1H), 4.16-4.11 (m, 2H), 4.09 (s, 2H), 3.92 (dd, J₁=3.6 Hz, J₂=10.8 Hz, 2H), 3.87-3.78 (m, 1H), 3.43 (t, J=11.2 Hz, 2H), 3.21-3.15 (m, 2H), 2.82 (s, 3H), 1.72-1.62 (m, 2H), 1.60-1.51 (m, 2H)

### Synthesis of 2-((5-(1-hydroxycyclopropyl)-N-methyl-1H-indazole)-7-sulfonamido)-N-(1-methyl-2-oxo-1,2-dihydropyridin-4-yl)acetamide (Example 172)

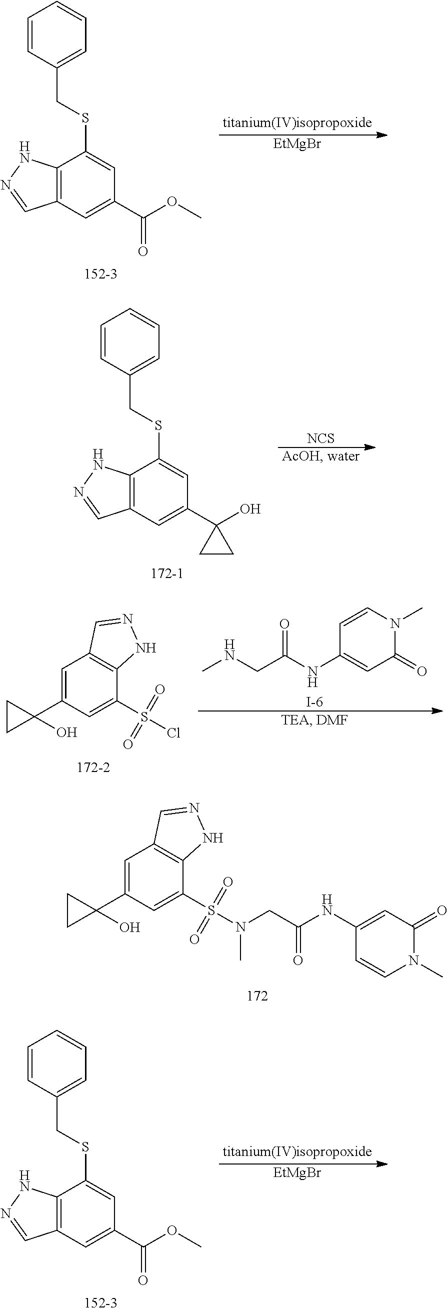

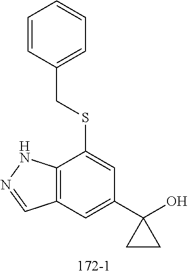 [SMILES: OC1(c2cc(SCc3ccccc3)c3[nH]ncc3c2)CC1]

To a mixture of 152-3 (0.500 g, 1.68 mmol) in tetrahydrofuran (6 mL) was added Ti(i-PrO)₄ (0.494 mL, 1.68 mmol), followed by EtMgBr (3 M, 2.23 mL) at 0° C. under nitrogen. The resulting mixture was stirred at 20° C. under a nitrogen atmosphere for 12 hours. The mixture was quenched with a saturate aqueous solution of ammonium chloride (50 mL) and extracted with ethyl acetate (40 mL*4). The combined organic layers were washed with brine (40 mL*2), dried over anhydrous sodium sulfate, filtered and the filtrate was concentrated under reduced pressure. The residue was purified by prep-HPLC (column: Phenomenex luna C₁₈ 150*40 mm*15 um; mobile phase: [water (0.225% FA)-CAN]; B %: 30%-60%, 10 min) the eluent was concentrated under reduced pressure to removed acetonitrile then extracted with ethyl acetate (100 mL*3). The combined organic layers were washed with brine (200 mL*3), dried over anhydrous sodium sulfate, filtered, and the filtrate was concentrated under reduced pressure to afford 1-(7-(benzylthio)-1H-indazol-5-yl) 260yclopropane-1-ol (172-1) (0.300 g, 59.1% yield) as a yellow oil.

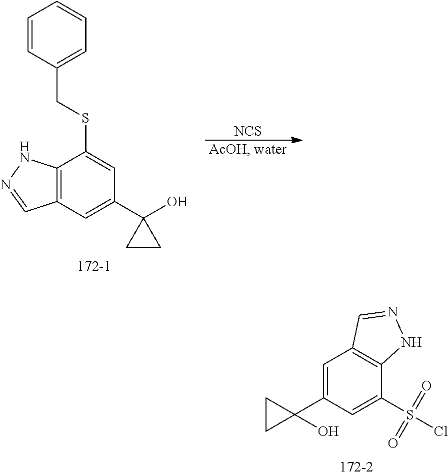 [SMILES: OC1(c2cc(SCc3ccccc3)c3[nH]ncc3c2)CC1 ; "AcOH, water" ; "NCS" ; O=S(=O)(Cl)c1cc(C2(O)CC2)cc2cn[nH]c12]

To a mixture of 172-1 (0.100 g, 0.337 mmol) in acetic acid (1 mL) and water (0.3 mL) was added NCS (0.180 g, 1.35 mmol) at 10° C. The mixture was stirred at 10° C. for 1 hour then poured into water (30 mL) and extracted with ethyl acetate (30 mL*3). The combined organic layers were washed with brine (30 mL*2), dried over anhydrous sodium sulfate, filtered and the filtrate was concentrated under reduced pressure to afford 5-(1-hydroxycyclopropyl)-1H-indazole-7-sulfonyl chloride (172-2) (0.092 g, crude) as yellow oil which was used directly.

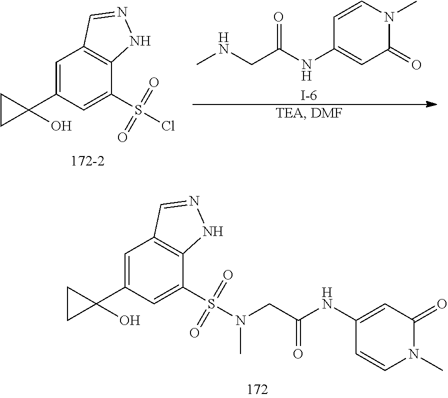

To a solution of I-6 (0.050 g, 0.256 mmol) in dimethylformamide (3 mL) was added diisopropyl ethylamine (0.178 mL, 1.02 mmol), followed by 172-2 (0.070 g, 0.256 mmol) at 0° C. The mixture was stirred at 20° C. for 0.5 hour then poured into water (30 mL) and extracted with n-butanol (30 mL*4). The combined organic layers were washed with brine (30 mL*2), dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (0.05% ammonia hydroxide v/v)-CAN]; B %: 4%-30%, 10 min) to afford 2-((5-(1-hydroxycyclopropyl)-N-methyl-1H-indazole)-7-sulfonamido)-N-(1-methyl-2-oxo-1,2-dihydropyridin-4-yl)acetamide (172) (0.009 g, 8% yield) as a white solid.

LCMS of 172: m/z 432.0 [M+H]⁺

¹H-NMR of 172: (DMSO-d₆+D₂O, 400 MHz): δ=8.20 (s, 1H), 7.93 (d, J=1.6 Hz, 1H), 7.69 (d, J=1.2 Hz, 1H), 7.56 (d, J=6.8 Hz, 1H), 6.63 (d, J=2.4 Hz, 1H), 6.33 (dd, J₁=2.4 Hz, J₁=6.8 Hz, 1H), 4.12 (s, 2H), 3.33 (s, 3H), 2.80 (s, 3H), 1.16-1.12 (m, 2H), 1.02-0.96 (m, 2H)

The following compounds were synthesized in a similar manner as 172 replacing I-6 with the reactant indicated.

| | | LCMS | |
|---|---|---|---|
| Example | Reactant | m/z | ¹H-NMR |
| 173 | I-37 | 514.1 | (CD₃OD, 400 MHz) δ 8.18 (s, 1H), 8.03 |
| | | | (d, J = 1.6 Hz, 1H), 7.90 (d, J = 1.6 Hz, |
| | | | 1H), 6.99 (d, J = 2.0 Hz, 1H), 6.88 (dd, |
| | | | J₁ = 2.4 Hz, J₂ = 8.8 Hz, 1H), 6.20 (d- |
| | | | J = 8.8 Hz, 1H), 4.-8-4.91 (m, 2H), |
| | | | 4.-0-4.76 (m, 2H), 4.-4-4.52 (m, 1H), |
| | | | 4.30-4.41 (m, -H), 4.07 (s, 2H), 3.18- |
| | | | 3.15 (m, -H), 2.86 (s, 3H), 1.-5-1.22 (m, |
| | | | 2H), 1.12-1.10 (m, 2H) |
| 174 | I-35 | 542.2 | (DMSO-d₆, 400 MHz): δ = 13.71 (br. s, |
| | | | 1H), 9.81 (br. s, 1H), 8.83 (s, 1H), 8.46 |
| | | | (s, -H), 8.25 (s, 1H), 7.-7-6.64 (m, 3H), |
| | | | 4.-8-4.04 (m, 4H), 3.-7-3.87 (m, 2H), |
| | | | 3.-7-3.75 (m, 1H), 3.-7-3.42 (m, 1H), |
| | | | 3.-8-3.09 (m, 3H), 2.-2-2.81 (m, 3H), |
| | | | 1.-3-1.50 (m, 4H), 1.25-1.05 (m, 4H) |

### Synthesis of 2-((5-(difluoromethyl)-3-fluoro-N-methyl-1H-indazole)-7-sulfonamido)-N-(4-(tetrahydro-2H-pyran-4-yl)-3,4-dihydro-2H-benzo[b][1,4]oxazin-7-yl)acetamide (Example 175)

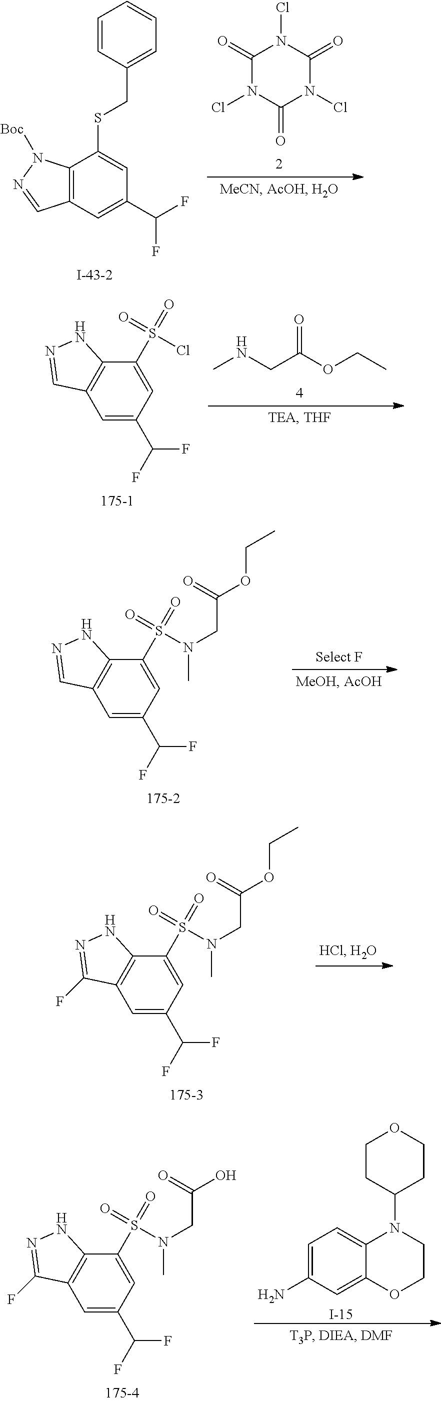

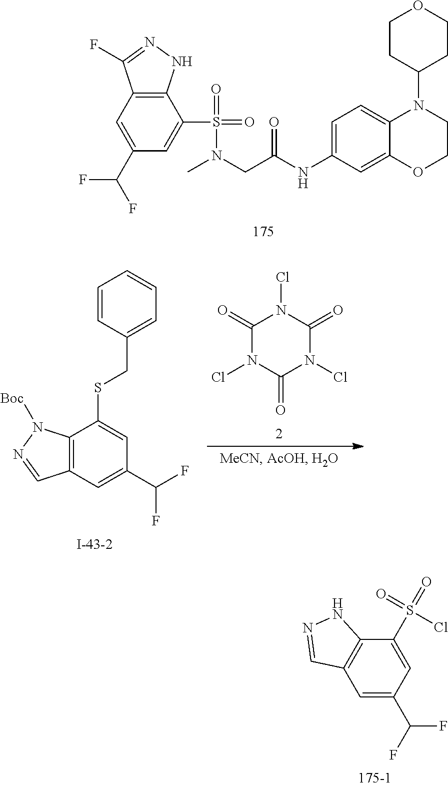 [SMILES: CN(CC(=O)Nc1ccc2c(c1)OCCN2C1CCOCC1)S(=O)(=O)c1cc(C(F)F)cc2c(F)n[nH]c12 ; CC(C)(C)OC(=O)n1ncc2cc(C(F)F)cc(SCc3ccccc3)c21 ; O=c1n(Cl)c(=O)n(Cl)c(=O)n1Cl ; O=S(=O)(Cl)c1cc(C(F)F)cc2cn[nH]c12]

To a solution of I-43-2 (1.00 g, 2.56 mmol) in a mixture of acetonitrile (10 mL), acetic acid (0.4 mL) and water (0.3 mL) was added 1,3,5-trichloro-1,3,5-triazinane-2,4,6-trione (1.19 g, 5.12 mmol) at 0° C. The reaction mixture was stirred at 0° C. for 2 hours then diluted with dichloromethane (100 mL) and washed with water (100 mL*3). The combined organic layers were washed with brine (100 mL*2), dried over anhydrous sodium sulfate, filtered, and the filtrate was concentrated reduced pressure to afford 5-(difluoromethyl)-1H-indazole-7-sulfonyl chloride (175-1) (0.680 g, crude) as yellow oil. No further purification was performed.

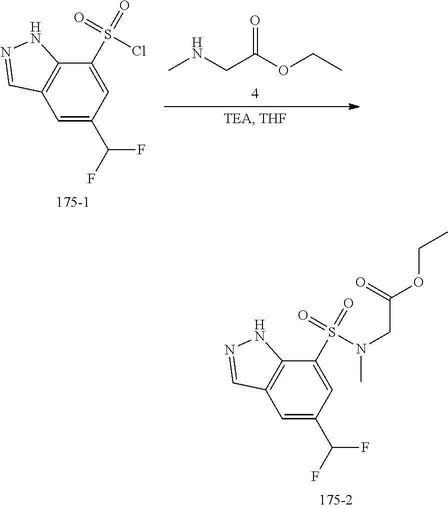

To a solution of ethyl 2-(methylamino)acetate; hydrochloride (0.392 g, 2.55 mmol) in tetrahydrofuran (10 mL) was added triethylamine (1.03 g, 10.2 mmol). The reaction mixture was stirred at 20° C. for 20 minutes then a solution of 175-1 (0.680 g, 2.55 mmol) in tetrahydrofuran (5 mL) was added drop wise at 20° C. The reaction mixture was stirred at 20° C. for 1 hour then filtered and the filtrate was concentrated reduced pressure directly. The residue was purified by silica gel chromatography (petroleum ether:ethyl acetate=3:1˜1:1) to afford ethyl 2-(5-(difluoromethyl)-N-methyl-1H-indazole-7-sulfonamido)acetate (175-2) (0.450 g, 50.8% yield) as a yellow oil.

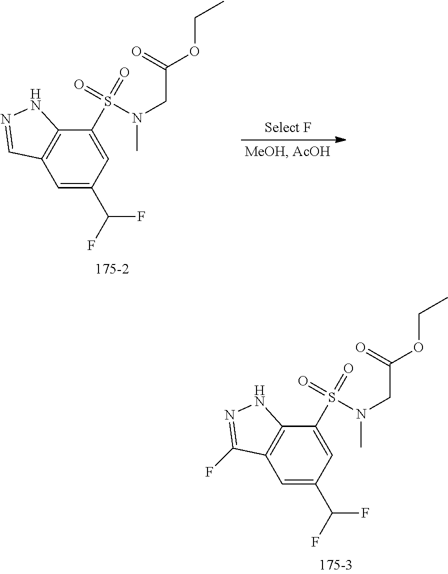 [SMILES: CCOC(=O)CN(C)S(=O)(=O)c1cc(C(F)F)cc2cn[nH]c12 ; CCOC(=O)CN(C)S(=O)(=O)c1cc(C(F)F)cc2c(F)n[nH]c12]

To a solution of 175-2 (0.500 g, 1.44 mmol) in acetonitrile (15 mL) was added acetic acid (7.35 g, 122 mmol) and 1-(chloromethyl)-4-fluoro-1,4-diazabicyclo[2.2.2]octane-1,4-diium tetrafluoroborate (1.02 g, 2.88 mmol) at 20° C. The reaction mixture was stirred at 90° C. for 28 hours under a nitrogen atmosphere. The mixture was diluted with water (100 mL) and extracted with ethyl acetate (100 mL*3). The combined organic layers were washed with brine (100 mL*2), dried over anhydrous sodium sulfate, filtered, and the filtrate was concentrated under reduced pressure. The residue was purified by silica gel chromatography (petroleum ether:ethyl acetate=2:1 to 1:1) to afford ethyl 2-(5-(difluoromethyl)-3-fluoro-N-methyl-1H-indazole-7-sulfonamido)acetate (175-3) (0.150 g, 28.5% yield) as a yellow oil.

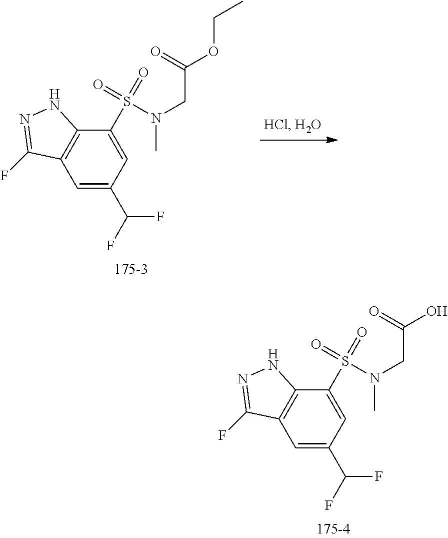 [SMILES: CCOC(=O)CN(C)S(=O)(=O)c1cc(C(F)F)cc2c(F)n[nH]c12 ; CN(CC(=O)O)S(=O)(=O)c1cc(C(F)F)cc2c(F)n[nH]c12]

A solution of 175-3 (0.160 g, 438 mmol) in a mixture of hydrochloric acid (3 M, 10 mL) and tetrahydrofuran (2 mL) was stirred at 60° C. for 8 hours. The mixture was diluted with water (100 mL) and extracted with ethyl acetate (100 mL*4). The combined organic layers were washed with brine (100 mL*2), dried over anhydrous sodium sulfate, filtered, and the filtrate was concentrated reduced pressure directly. The residue was purified by reversed-phase flash chromatography (0.1% TFA/MeCN condition) to afford 2-(5-(difluoromethyl)-3-fluoro-N-methyl-1H-indazole-7-sulfonamido)acetic acid (175-4) (0.120 g, 81.2% yield) as a white solid.

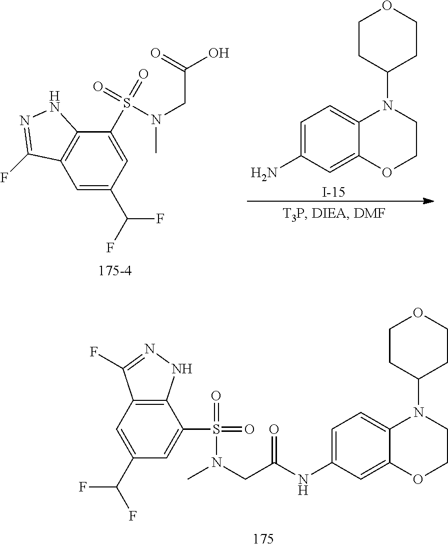

To a mixture of 175-4 (0.070 g, 0.21 mmol) and I-15 (0.073 g, 0.31 mmol) in N, N-dimethylformamide (3 mL) was added N, N-diisopropylethylamine (0.107 g, 0.830 mmol) and 2,4,6-tripropyl-1,3,5,2,4,6-trioxatriphosphinane 2,4,6-trioxide (0.264 g, 0.415 mmol 50% w/w in ethyl acetate) at 0° C. The reaction mixture was stirred at 20° C. for 3 hours then diluted with water (100 mL) and extracted with ethyl acetate (100 mL*3). The combined organic layers were washed with brine (100 mL*2), dried over anhydrous sodium sulfate, filtered, and the filtrate was concentrated under reduced pressure. The residue was purified by prep-HPLC (column: Phenomenex Gemini-NX Cis 75*30 mm*3 um; mobile phase: [water (10 mM NH₄HCO₃)-ACN]; B %: 35%-65%, 8 min) to afford 2-(5-(difluoromethyl)-3-fluoro-N-methyl-1H-indazole-7-sulfonamido)-N-(4-(tetrahydro-2H-pyran-4-yl)-3,4-dihydro-2H-benzo[b][1,4]oxazin-7-yl)acetamide (175) (0.018 g, 16% yield) as an off-white solid.

LCMS of 175: m/z: 554.4[M+H]⁺

¹H-NMR of 175: (DMSO-d₆, 400 MHz): δ13.15 (br. s, 1H), 9.79 (br. s, 1H), 8.35 (s, 1H), 8.01 (s, 1H), 7.21 (t, −J=55.6 Hz, 1H), 6.95-6.85 (m, 2H), 6.77 (d-J=9.2 Hz, 1H), 4.15-4.10 (m, 4H), 3.92 (dd, J₁=4.0 Hz, -₂=10.8 Hz, 2H), 3.-6-3.78 (m, 1H), 3.47-3.40 (m, 2H), 3.18 (t, J=4.4 Hz, —H), 2.84 (s, 3H), 1.-2-1.62 (m, 2H), 1.59-1.53 (m, 2H)

### Synthesis of 2-((3-chloro-N,5-dimethyl-1H-indazole)-7-sulfonamido)-N-(1-methyl-2-oxo-1,2-dihydropyridin-4-yl)acetamide (Example 176)

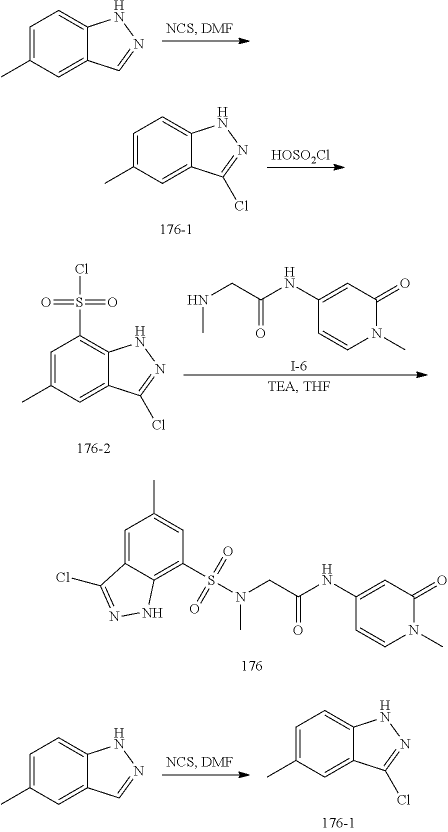

To a solution of 5-methyl-1H-indazole (0.500 g, 3.78 mmol) in dimethylformamide (5 mL) at 0° C. was added NCS (1.01 g, 7.57 mmol). The mixture was stirred at 25° C. for 12 hours then diluted with water (50 mL) and extracted with ethyl acetate (50 mL*3). The combined organic layers were washed by brine (100 mL*3), dried over anhydrous sodium sulfate, filtered, and the filtrate was concentrated under reduced pressure. The residue was combined with another batch (0.100 g scale) and purified by column chromatography (SiO₂, petroleum ether/ethyl acetate=10/1) to afford 3-chloro-5-methyl-1H-indazole (176-1) (0.475 g, 60.2% yield) as a white solid.

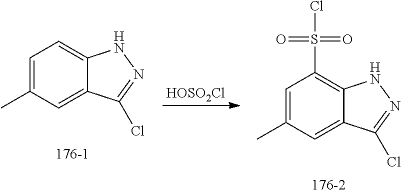

To sulfurochloridic acid (0.887 mL, 13.3 mmol) was added 176-1 (0.072 g, 0.038 mmol). The mixture was stirred at 25° C. for 3 hours then added to ice-water (20 mL) dropwise, causing a solid to precipitate. The formed solid was collected by filtration and the filter cake was redissolved in ethyl acetate (20 mL), dried over anhydrous sodium sulfate, filtered, and the filtrate was concentrated under reduced pressure to afford 3-chloro-5-methyl-1H-indazole-7-sulfonyl chloride (176-2) (0.050 g, 49.2% yield) as a gray solid. No further purification was performed.

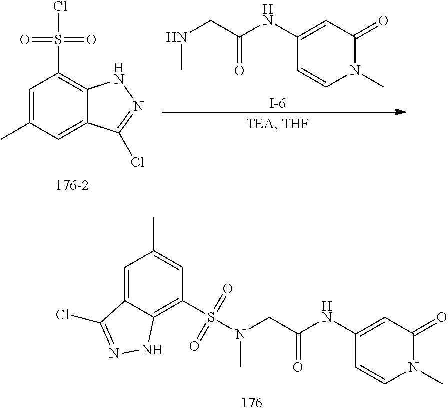

To a mixture of I-6 as the trifluoroacetic salt (0.058 g, 0.189 mmol) and triethylamine (0.100 mL, 0.718 mmol) in tetrahydrofuran (1 mL) at 0° C. was added 176-2 (0.050 g, 0.189 mmol) in tetrahydrofuran (0.5 mL). The mixture was stirred at 25° C. for 1 hour then diluted with water (4 mL) causing a solid to precipitate. The formed solid was collected by filtration and triturated with acetonitrile (2 mL*2) to afford 2-((3-chloro-N,5-dimethyl-1H-indazole)-7-sulfonamido)-N-(1-methyl-2-oxo-1,2-dihydropyridin-4-yl)acetamide (176) (0.017 g, 21.8% yield) as a white solid

LCMS of 176: m/z=423.9 [M+H]⁺

¹H-NMR of 176: (DMSO-d₆, 400 MHz) δ 13.37 (br. s, 1H), 10.13 (br. s, 1H), 7.80 (s, 1H), 7.72 (d, J=1.2 Hz, 1H), 7.58 (d, J=7.6 Hz, 1H), 6.58 (d, J=2.0 Hz, 1H), 6.27 (dd, J₁=2.0 Hz, J₂=7.6 Hz, 1H), 4.13 (s, 2H), 3.33 (s, —H), 2.86 (s, 3H), 2.49-2.48 (m, 3H)

### Synthesis of 2-((5-(difluoromethyl)-3-fluoro-N-methyl-1H-indazole)-7-sulfonamido)-N-(4-(tetrahydro-2H-pyran-4-yl)-3,4-dihydro-2H-benzo[b][1,4]oxazin-7-yl)acetamide (Example 175)

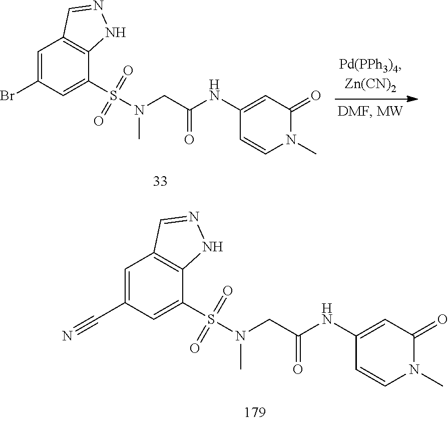 [SMILES: CN(CC(=O)Nc1ccn(C)c(=O)c1)S(=O)(=O)c1cc(Br)cc2cn[nH]c12 ; CN(CC(=O)Nc1ccn(C)c(=O)c1)S(=O)(=O)c1cc(C#N)cc2cn[nH]c12]

To a mixture of 33 (0.100 g, 0.181 mmol) and zinc cyanide (0.085 g, 0.725 mmol) in N, N-dimethylformamide (1.5 mL) was added tetrakis(triphenylphosphine)palladium (0) (0.042 g, 0.036 mmol) under nitrogen atmosphere in a microwave tube. The mixture was heated at 150° C. for 268ydroxyamider microwave conditions. The mixture was diluted with water (40 mL) and extracted with ethyl acetate (30 mL*5). The combined organic layers were washed with brine (80 mL), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH₄HCO₃)-ACN]; B %: 10%-40%, 10 min) and further purified by prep-HPLC (column: 3_Phenomenex Luna C₁₈ 75*30 mm*3 um; mobile ph-se: [water (0.05% HCl)-ACN]; B %: 18%-38%, 6.5 min) to afford 2-(N,5-dimethyl-1H-indazole-7-sulfonamido)-N-((1-methyl-1H-1,2,3-triazol-4-yl)methyl)acetamide (179) (0.013 g, 17.8% yield) as a white solid.

LCMS of 179: m/z 401.1 [M+H]⁺.

¹H-NMR of 179: (DMSO-d₆, 400 MHz): δ 13.73 (br. s, 1H), 10.26 (br. s, 1H), 8.75 (d, J=1.2 Hz, 1H), 8.47 (s, 1H), 8.10 (d, J=1.6 Hz, 1H), 7.58 (d-J=7.6 Hz, 1H), 6.-6-6.55 (m, 1H), 6.29-6.26 (m, 1H), 4.24 (s, 2H), 3.33 (s, 3H), 2.91 (s, 3H)

### Synthesis of (S)-1-((5-(difluoromethyl)-1H-indazol-7-yl)sulfonyl)-N-(4-(oxetan-3-yl)-3,4-dihydro-2H-benzo[b][1,4]oxazin-7-yl)azetidine-2-carboxamide (Example 180)

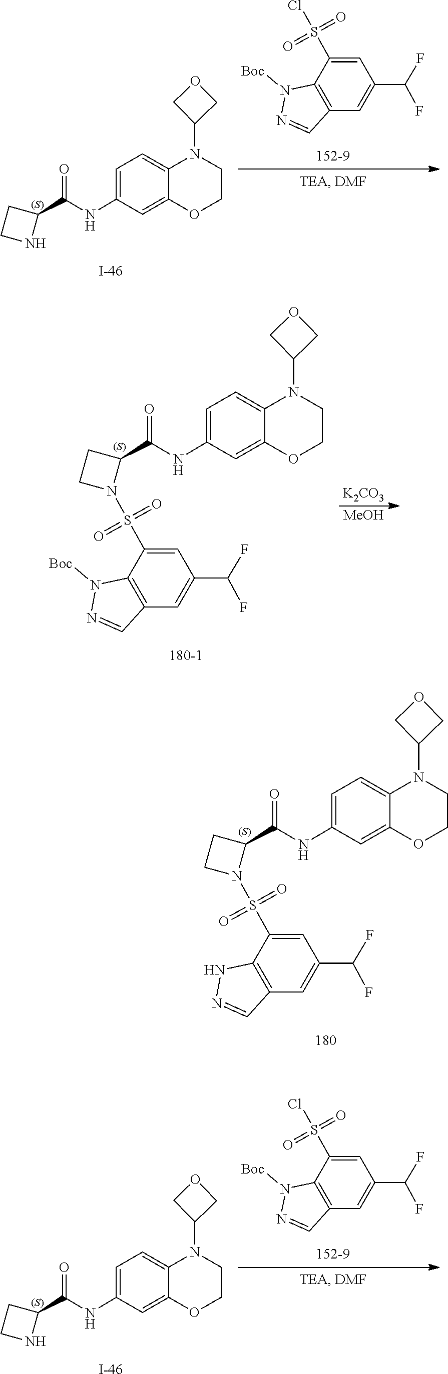

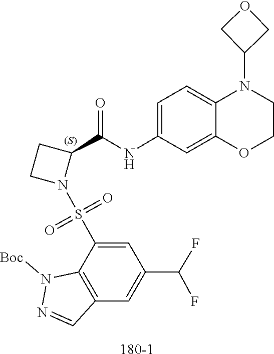

To a mixture of I-46 (0.030 g, 0.104 mmol) and triethylamine (0.060 mL, 0.431 mmol) in dimethylformamide (1 mL) at 0° C. was added 152-9 (0.056 g, 0.104 mmol). The mixture was stirred at 0° C. for 1 hour then added to ice-water (20 mL) and extracted with ethyl acetate (20 mL*3). The combined organic layers were washed with brine (40 mL*3), dried over anhydrous sodium sulfate, filtered, and the filtrate was concentrated under reduced pressure to afford tert-butyl (S)-5-(difluoromethyl)-7-((2-((4-(oxetan-3-yl)-3,4-dihydro-2H-benzo[b][1,269ydroxyam-7-yl)carbamoyl)azetidin-1-yl)sulfonyl)-1H-indazole-1-carboxylate (180-1) (0.060 g, crude) as yellow oil. No further purification was performed.

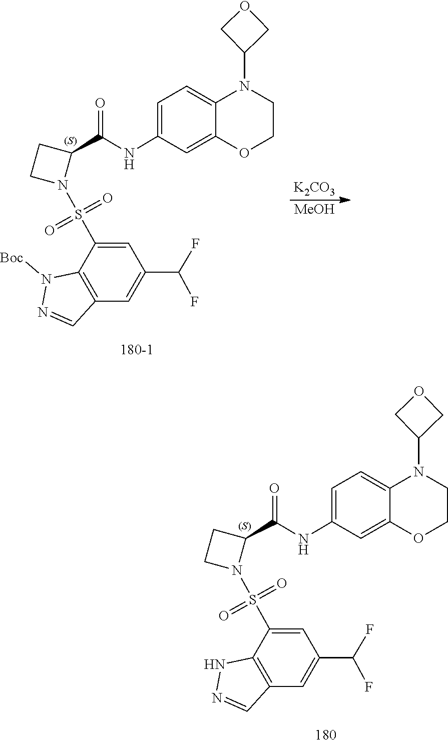

A mixture of 180-1 (0.060 g, 0.097 mmol) and potassium carbonate (0.002 g, 0.014 mmol) in methanol (1 mL) was stirred at 25° C. for 3 hours. The mixture was diluted with water (20 mL) and extracted with ethyl acetate (20 mL*3). The combined organic layers were washed with brine (50 mL*3), dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (0.05% ammonia hydroxide v/v)-ACN]; B %: 20%-50%, 7 min) to afford (S)-1-((5-(difluoromethyl)-1H-indazol-7-yl)sulfonyl)-N-(4-(oxetan-3-yl)-3,4-dihydro-2H-benzo[b][1,4]oxazin-7-yl)azetidine-2-carboxamide (180) (0.0060 g, 11% yield) as a white solid.

LCMS of 180: m/z=520.0 [M+H]⁺

¹-NMR of 180: (DMSO-d₆, 400 MHz) δ 10.04 (br. s, 1H), 8.48 (s, 1H), 8.46 (d, J=0.8 Hz, —H), 8.00 (s, 1H), 7.40-7.10 (m, 2H), 6.98 (d, J=8.8 Hz, 1H), 6.31 (d, J=8.8 Hz, 1H), 4.95 (t-J=8.0 Hz, 1H), 4.81-4.73 (m, 2H), 4.67 (t-J=6.4 Hz, 2H), 4.-7-4.50 (m, 1H), 4.-4-4.25 (m, 2H), 3.-2-3.83 (m, 1H), 3.-4-3.57 (m, 1H), 3.-2-3.14 (m, 2H), 2.44-2.35 (m, 2H)

### Synthesis of 2-((2-cyano-N,5-dimethyl-1H-indole)-7-sulfonamido)-N-(4-(tetrahydro-2H-pyran-4-yl)-3,4-dihydro-2H-benzo[b][1,4]oxazin-7-yl)acetamide (Example 181)

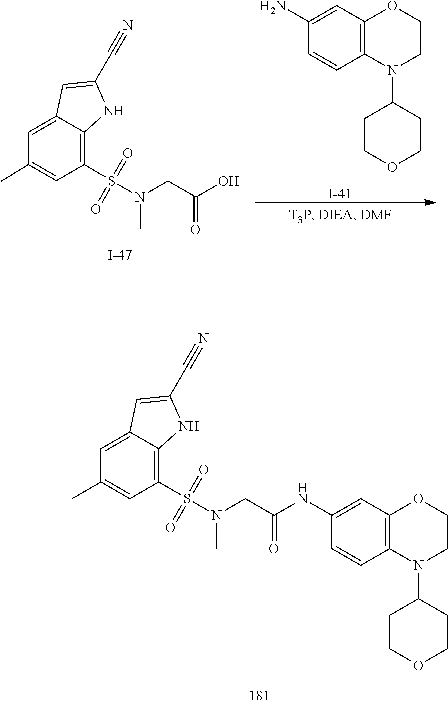

To a mixture of I-47 (0.020 g, 0.065 mmol), diisopropylethylamine (0.016 g, 0.130 mmol) and I-41 (0.015 g, 0.065 mmol) in dimethylformamide (1 mL) was added T₃P (0.124 g, 0.195 mmol, 50% w/w in ethyl acetate) under nitrogen. The mixture was stirred at 25° C. for 1 hour then poured into ice-water (20 mL), stirred for 5 minutes, and extracted with ethyl acetate (100 mL*3). The combined organic layers were washed with brine (20 mL*3), dried over anhydrous sodium sulfate, filtered, and the filtrate was concentrated under reduced pressure. The residue was purified by prep-HPLC (column: Waters Xbridge 150*25 mm*5 um; mobile phase: [water (10 mM NH₄HCO₃)-ACN]; B %: 44%-74%, 10 min) to afford 2-((2-cyano-N,5-dimethyl-1H-indole)-7-sulfonamido)-N-(4-(tetrahydro-2H-pyran-4-yl)-3,4-dihydro-2H-benzo[b][1,4]oxazin-7-yl)acetamide (181) (4.60 mg, 13.5% yield) as a white solid.

LCMS of 181: m/z=524.2 [M+H]⁺

¹H-NMR of 181: (CDCl₃, 400 MHz) δ 10.79 (br. s, 1H), 7.75 (s, 2H), 7.66 (s, 1H), 7.19 (s, 1H), 7.10 (dd, J₁₌2.8 Hz, J₂=8.8 Hz, 1H), 6.97 (d, J=2.4 Hz, 1H), 6.74 (d, J=8.8 Hz, 1H), 4.24 (t-J=4.4 Hz, 2H), 4.12-4.08 (m, —H), 3.94 (s, 2H), 3.-5-3.81 (m, 1H), 3.57-3.49 (m, 2H), 3.27 (t, J=4.4 Hz, 2H), 2.85 (s, —H), 2.53 (s, 3H), 1.83-1.75 (m, 4H)

The following compound was synthesized in a similar manner as 181 replacing I-41 with the reactant indicated.

| | | LCMS | |
|---|---|---|---|
| Example | Reactant | m/z | ¹H-NMR |
| 189 | I-44 | 496.1 | (DMSO-d6, 400 MHz) δ 12.37 (br. s, 1H), |
| | | | 9.81 (br. s, 1H), 7.81 (s, 1H), 7.62 (s, 1H), |
| | | | 7.47 (s, 1H), 7.01 (d, J = 2.4 Hz, 1H), 6.87 |
| | | | (dd, J1 = 2.4 Hz, J2 = 8.6 Hz, 1H), 6.23 (d, |
| | | | J = 8.8 Hz, 1H), 4.75 (t, J = 6.8 Hz, 2H), 4.64 |
| | | | (t-J = 6.8 Hz, 2H), 4.-3-4.48 (m, 1H), 4.28- |
| | | | 4.25 (m, −H), 4.00 (s, 2H), 3.15-3.13 (m, |
| | | | 2H), 2.78 (s, 3H), 2.49 (s, 3H) |
| 198 | 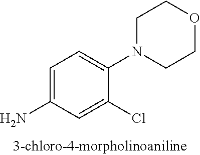 [SMILES: Nc1ccc(N2CCOCC2)c(Cl)c1] | 502.0 | (DMSO-d6, 400 MHz): δ 12.26 (br, s, 1H), 10.13 (br, s, 1H), 7.81 (s, 1H), 7.68 (d, J = 2.8 Hz, 1H), 7.62 (s, 1H), 7.46 (s, 1H), 7.36 (dd, J1 = 2.4 Hz , J2 = 8.8 Hz, 1H), 7.12 (d, J = 8.8 Hz, 1H), 4.07 (s, 2H), 3.73 (t, J = 4.4 Hz, 4H), 2.91 (t, J = 4.4 Hz, 4H), 2.83 (s, 3H), 2.43 (s, 3H) |

### Synthesis of 2-((3-cyano-N,5-dimethyl-1H-indazole)-7-sulfonamido)-N-(1-methyl-2-oxo-1,2-dihydropyridin-4-yl)acetamide (Example 182)

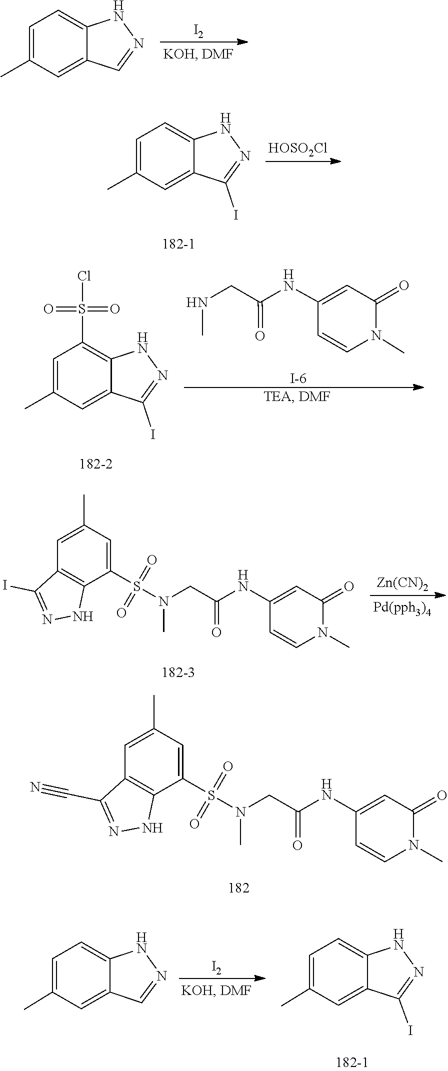

A mixture of 5-methyl-1H-indazole (0.300 g, 2.27 mmol), iodine (0.915 mL, 4.54 mmol) and potassium hydroxide (0.509 g, 9.08 mmol) in dimethylformamide (2 mL) was stirred at 25° C. for 3 hours. The mixture was quenched by addition of a saturated aqueous solution of sodium bisulfite (50 mL) at 25° C. then extracted with ethyl acetate (100 mL*3). The combined organic layers were washed with brine (100 mL*2), dried over anhydrous sodium sulfate, filtered, and the filtrate was concentrated under reduced pressure. The residue was purified by column chromatography (SiO₂, petroleum ether/ethyl acetate=1/0 to 0/1) to afford 3-iodo-5-methyl-1H-indazole (182-1) (0.360 g, 1.29 mmol, 56.9% yield) as a yellow solid.

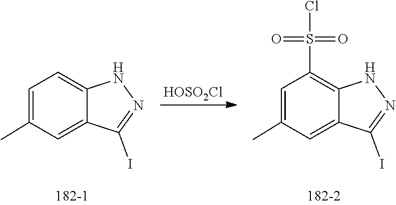

To a solution of chlorosulfonic acid (1.5 mL, 22.5 mmol) was added 182-1 (0.100 g, 0.390 mmol) at 0° C. The mixture was stirred at 70° C. under nitrogen for 3 hours. The reaction mixture was added to ice-water (50 mL), quenched with a saturated aqueous solution of sodium bisulfite solution (50 mL) at 25° C., and extracted with ethyl acetate (100 mL*3). The combined organic layers were washed with brine (100 mL*2), dried over anhydrous sodium sulfate, filtered, and the filtrate was concentrated under reduced pressure to afford 3-iodo-5-methyl-1H-indazole-7-sulfonyl chloride (182-2) (0.110 g, 72.3% yield) as a yellow solid.

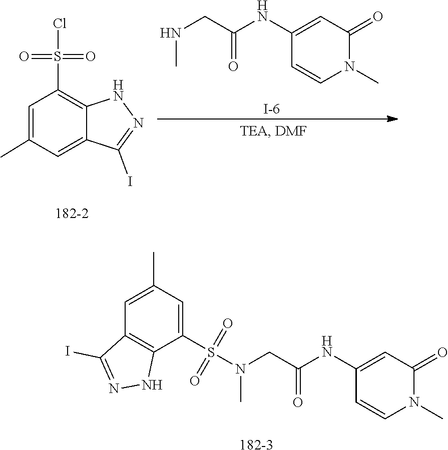

To a solution of 182-2 (0.039 g, 0.130 mmol) in dimethylformamide (0.5 mL) was added triethylamine (0.089 mL, 0.640 mmol) at 0° C. The mixture was stirred at 0° C. for 20 minutes, then I-6 (0.050 g, 0.130 mmol) was added to the mixture and the mixture was stirred at 25° C. for 40 minutes then diluted with water (20 mL) and extracted with ethyl acetate (30 mL*3). The combined organic layers were washed with brine (20 mL*2), dried over anhydrous sodium sulfate, filtered, and the filtrate was concentrated under reduced pressure to afford 2-(3-iodo-N,5-dimethyl-1H-indazole-7-sulfonamido)-N-(1-methyl-2-oxo-1,2-dihydropyridin-4-yl)acetamide (182-3) (0.020 g, 28.3% yield) as a yellow solid.

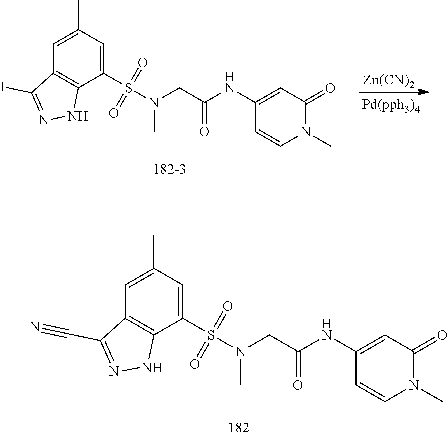

To a mixture of 182-3 (0.060 g, 0.089 mmol) and zinc cyanide (0.017 mL, 0.270 mmol) in dimethylformamide (1 mL) was added Pd(PPh₃)₄ (0.021 g, 0.018 mmol) under nitrogen atmosphere in a microwave tube. The mixture was heated at 130° C. for 2 hours at microwave condition. The mixture was quenched by water (30 mL), and extracted with ethyl acetate (50 mL*3). The combined organic layers were washed with brine (50 mL*2), dried over anhydrous sodium sulfate, filtered and the filtrate was concentrated under reduced pressure. The residue was purified by reversed-phase flash (0.1% HCl/MeCN condition), followed by prep-HPLC (column: Phenomenex Gemini-NX Cis 75*30 mm*3 um; mobile phase: [water (10 mM NH₄HCO₃)-ACN]; B %: 16%-46%, 8 min) to afford 2-(3-cyano-N,5-dimethyl-1H-indazole-7-sulfonamido)-N-(1-methyl-2-oxo-1,2-dihydropyridin-4-yl)acetamide (182) (4.65 mg, 12.4% yield) as a white solid.

LCMS of 182: m/z 415.1 [M+H]⁺

¹H-NMR of 182: (CD₃OD, 400 MHz) δ 7.92 (s, 1H), 7.83 (s, 1H), 7.55 (d, J=8.4 Hz, 1H), 6.77 (d, J=2.0 Hz, 1H), 6.53 (dd, J₁=2.4 Hz, J₂=7.6 Hz, 1H), 4.20 (s, 2H), 3.50 (s, 3H), 2.95 (s, 3H), 2.57 (s, 3H)

### Synthesis of 2-((5-(difluoromethyl)-3-fluoro-N-methyl-1H-indazole)-7-sulfonamido)-N-(1-methyl-2-oxo-1,2-dihydropyridin-4-yl)acetamide (Example 183)

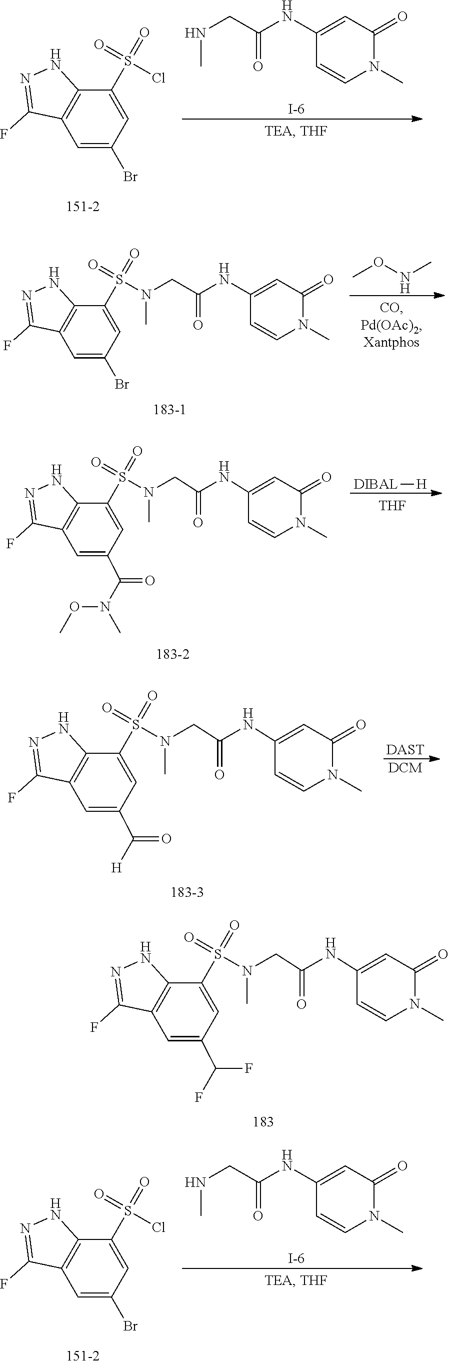

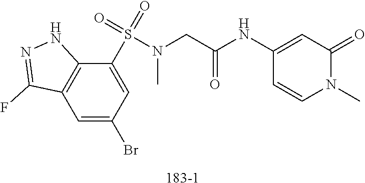 [SMILES: CN(CC(=O)Nc1ccn(C)c(=O)c1)S(=O)(=O)c1cc(Br)cc2c(F)n[nH]c12 ; "183-1"]

To a mixture of I-6 (0.493 g, 1.59 mmol) in dimethylformamide (10 mL) was added triethylamine (1.11 mL, 7.97 mmol), followed by 151-2 (0.500 g, 1.59 mmol) at 0° C. The mixture was stirred at 0° C. for 1 hour. The mixture was poured into water (80 mL), causing a solid to precipitate. The formed solid was collected by filtration and triturated with ethyl acetate (20 mL) to afford 2-((5-bromo-3-fluoro-N-methyl-1H-indazole)-7-sulfonamido)-N-(1-methyl-2-oxo-1,2-dihydropyridin-4-yl)acetamide (183-1) (0.700 g, 92.8% yield) as a white solid. No further purification was performed.

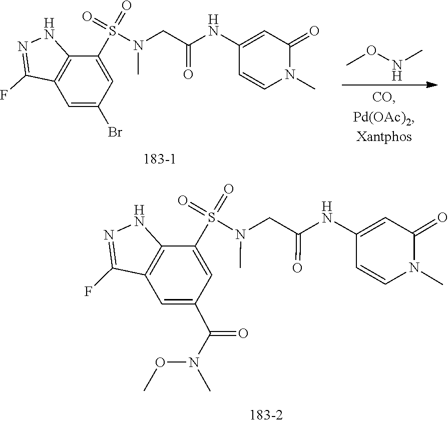

To a mixture of 183-1 (0.700 g, 1.48 mmol) in acetonitrile (15 mL) was added N-methoxymethanamine hydrochloric salt (0.289 g, 2.96 mmol), followed by diisopropylethylamine (1.81 mL, 10.4 mmol) and Xantphos (0.086 g, 0.148 mmol). The mixture was degassed with nitrogen for three times, then Pd(OAc)₂ (0.017 g, 0.074 mmol) was added to the mixture. The mixture was stirred at 80° C. under carbon monoxide (50 psi) for 12 hours. The mixture was filtered and the filtrate was concentrated under reduced pressure. The residue was taken back up with N-methoxymethanamine hydrochloric salt (0.223 g, 2.29 mmol) in dimethylformamide (10 mL). To this was added diisopropylethylamine (0.597 mL, 3.43 mmol) followed by T₃P (0.509 mL, 1.71 mmol, 50% w/w in ethyl acetate) drop-wise. The mixture was stirred at 20° C. for 1 hour then diluted with water (60 mL) and extracted with ethyl acetate (30 mL*3). The combined organic layers were washed with brine (30 mL*2), dried over anhydrous sodium sulfate, filtered, and the filtrate was concentrated under reduced pressure. The residue was purified by prep-HPLC (column: Phenomenex luna C₁₈ 150*40 mm*15 um; mobile phase: [water (0.1% TFA)-ACN]; B %: 16%-46%, 11 min) to afford 3-fluoro-N-methoxy-N-methyl-7-(N-methyl-N-(2-((1-methyl-2-oxo-1,2-dihydropyridin-4-yl)amino)-2-oxoethyl)sulfamoyl)-1H-indazole-5-carboxamide (183-2) (0.300 g, 0.624 mmol, 54.6% yield) as a white solid.

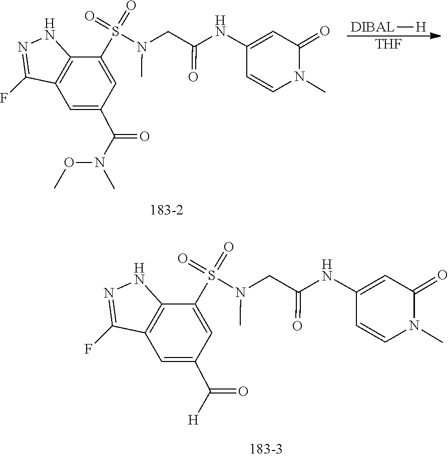

To a mixture of 183-2 (0.300 g, 0.624 mmol) in tetrahydrofuran (4 mL) was added DIBAL-H (1 M, 3.12 mL) drop-wise at −70° C. under nitrogen atmosphere. The result mixture was stirred at −70° C. under nitrogen atmosphere for 1 hour. The mixture was added to a cooled saturated aqueous solution of ammonium chloride (50 mL), filtered and rinsed with acetonitrile (150 mL). The filtrate was concentrated to 70 mL and extracted with ethyl acetate (30 mL*3). The combined organic layers were washed with brine (30 mL*2), dried over anhydrous sodium sulfate, filtered, and the filtrate was concentrated under reduced pressure. The residue was purified by prep-HPLC (column: Phenomenex Synergi C₁₈ 150*25 mm*10 um; mobile phase: [water (0.1% TFA)-ACN]; B %: 21%-51%, 10 min) and triturated with ethyl acetate (5 mL) to afford 2-((3-fluoro-5-formyl-N-methyl-1H-indazole)-7-sulfonamido)-N-(1-methyl-2-oxo-1,2-dihydropyridin-4-yl)acetamide (183-3) (0.040 g, 0.095 mmol, 26.7% yield) as a white solid.

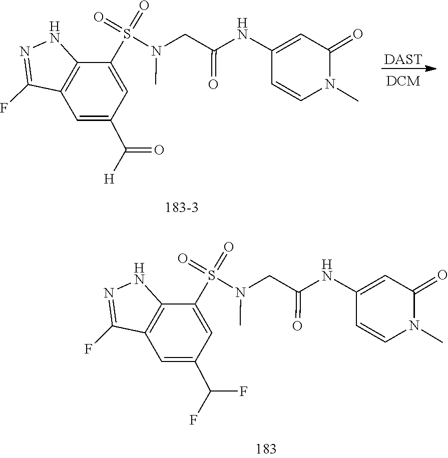

To a mixture 183-3 (0.030 g, 0.071 mmol) in dichloromethane (1 mL) was added DAST (0.069 g, 0.427 mmol) at 0° C. potion-wise. The mixture was stirred at 0° C. for 2 hours then stirred at 20° C. for 6 hours then diluted with a saturated aqueous solution of sodium bicarbonate (30 mL) and extracted with ethyl acetate (20 mL*3). The combined organic layers were dried over anhydrous sodium sulfate, filtered, and the filtrate was concentrated under reduced pressure. The residue was purified by prep-HPLC (column: 3_Phenomenex Luna C₁₈ 75*30 mm*3 um; mobile phase: [water (0.1% TFA)-ACN]; B %: 34%-54%, 7 min) to afford 2-((5-(difluoromethyl)-3-fluoro-N-methyl-1H-indazole)-7-sulfonamido)-N-(1-methyl-2-oxo-1,2-dihydropyridin-4-yl)acetamide (183) (0.006 g, 9.97% yield) as a white solid.

LCMS of 183: m/z 444.2 [M+H]⁺

¹H-NMR of 183: (CD₃OD, 400 MHz): δ=8.21 (d, J=1.2 Hz, 1H), 8.04 (s, 1H), 7.55 (d-J=7.6 Hz, 1H), 7.12-6.82 (m, 1H), 6.78 (d, J=2.0 Hz, 1H), 6.54 (dd, J₁₌2.4 Hz, J₂=7.2 Hz, 1H), 4.24 (s, 2H), 3.50 (s, 3H), 2.98 (s, 3H)

### Synthesis of (S)-1-((5-(difluoromethyl)-3-fluoro-1H-indazol-7-yl)sulfonyl)-N-(4-(oxetan-3-yl)-3,4-dihydro-2H-benzo[b][1,4]oxazin-7-yl)azetidine-2-carboxamide (Example 184)

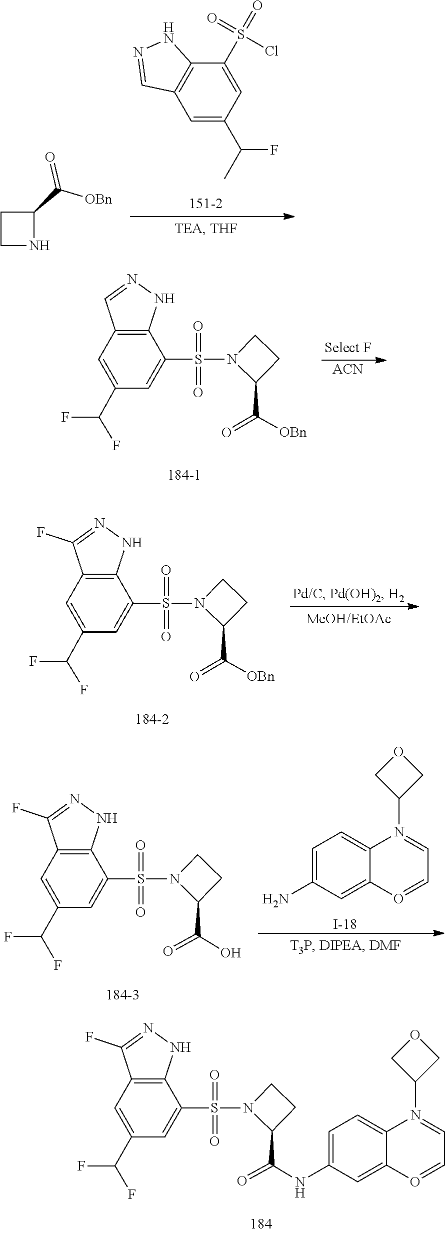

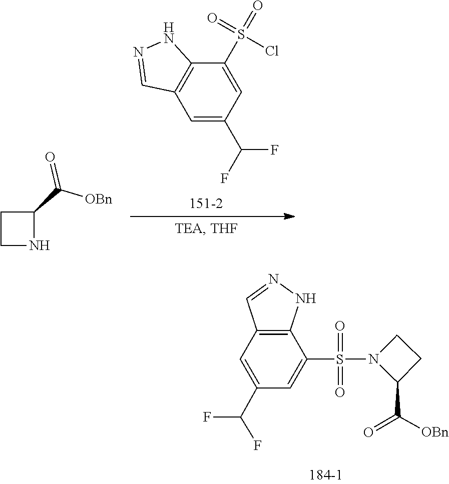 [SMILES: O=C(OCc1ccccc1)[C@@H]1CCN1 ; O=S(=O)(Cl)c1cc(C(F)F)cc2cn[nH]c12 ; O=C(OCc1ccccc1)[C@@H]1CCN1S(=O)(=O)c1cc(C(F)F)cc2cn[nH]c12]

A mixture of benzyl (S)-azetidine-2-carboxylate as the trifluoroacetic salt (1.30 g, 4.26 mmol) and triethylamine (1.29 g, 12.7 mmol) in tetrahydrofuran (20 mL) was stirred at 25° C. for 5 minutes, then 151-2 (1.68 g, 4.26 mmol) was added and stirred for 55 minutes at 25° C. The mixture was poured into ice-water (50 mL) and stirred for 5 minutes. The aqueous phase was extracted with ethyl acetate (150 mL*3). The combined organic layers were washed with brine (10 mL*3), dried over anhydrous sodium sulfate, filtered, and the filtrate was concentrated under reduced pressure. The residue was purified by reversed-phase flash (0.1% TFA/MeCN condition) to afford benzyl (S)-1-((5-(difluoromethyl)-1H-indazol-7-yl)sulfonyl)azetidine-2-carboxylate (184-1) (0.400 g, 41.8% yield) as a yellow solid.

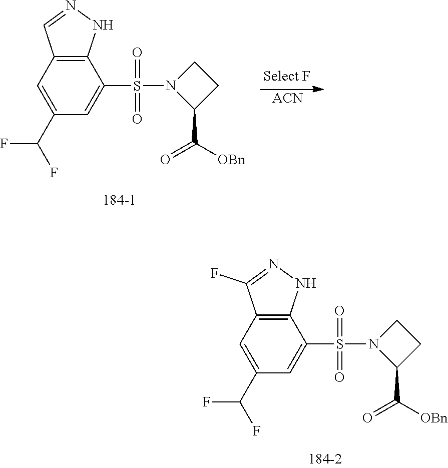

A mixture of 184-1 (0.350 g, 0.830 mmol), and Select F (1.47 g, 4.15 mmol) in acetonitrile (10 mL) was taken up into a microwave tube. The sealed tube was heated at 125° C. for 60 minutes under microwave conditions. The mixture was combined with another batch (0.350 g scale), poured into ice-water (10 mL) and stirred for 5 minutes. The aqueous phase was extracted with ethyl acetate (150 mL*4). The combined organic layers were washed with brine (20 mL*2), dried over anhydrous sodium sulfate, filtered, and the filtrate was concentrated under reduced pressure. The residue was purified by silica gel chromatography (column height: 250 mm, diameter: 100 mm, 100-200 mesh silica gel, petroleum ether/ethyl acetate=20/1 to 2/1) to afford benzyl (S)-1-((5-(difluoromethyl)-3-fluoro-1H-indazol-7-yl)sulfonyl)azetidine-2-carboxylate (184-2) (0.200 g, 52.7% yield) as yellow gum.

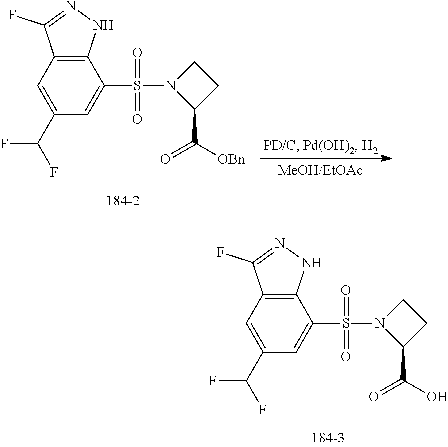 [SMILES: O=C(OCc1ccccc1)[C@@H]1CCN1S(=O)(=O)c1cc(C(F)F)cc2c(F)n[nH]c12 ; O=C(O)[C@@H]1CCN1S(=O)(=O)c1cc(C(F)F)cc2c(F)n[nH]c12]

To a solution of 184-2 (0.170 g, 0.386 mmol) in a mixture of methanol (2 mL)/ethyl acetate (2 mL) was added Pd(OH)₂ (0.020 g, 0.071 mmol, 50% on charcoal, wet) and Pd/C (0.020 g, 10% on charcoal, wet) under nitrogen. The suspension was degassed and purged with hydrogen three times. The mixture was stirred at hydrogen atmosphere (15 psi) at 25° C. for 1 hour. The mixture was filtered and the filtrate was concentrated under reduced pressure to afford (S)-1-((5-(difluoromethyl)-3-fluoro-1H-indazol-7-yl)sulfonyl)azetidine-2-carboxylic acid (184-3) (0.080 g, 0.225 mmol, 58.3% yield) as a yellow solid.

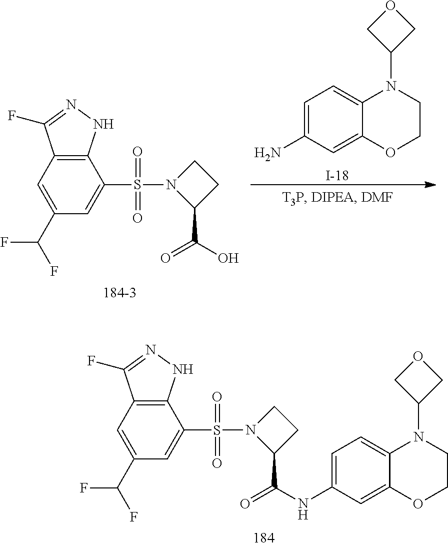

To a mixture of 184-3 (0.020 g, 0.057 mmol) and I-18 (0.023 g, 0.114 mmol), diisopropylethylamine (0.022 g, 0.171 mmol) in dimethylformamide (1 mL) was added T₃P (0.091 g, 0.143 mmol, 50% w/w in ethyl acetate). The mixture was stirred at 25° C. for 1 hour then combined with another batch (0.010 g scale), poured into ice-water (50 mL), and stirred for 5 minutes. The aqueous phase was extracted with ethyl acetate (100 mL*3) and the combined organic layers were washed with brine (40 mL*3), dried over anhydrous sodium sulfate, filtered, and the filtrate was concentrated under reduced pressure. The residue was purified by reversed-phase flash (0.1% NH₃·H₂O/MeCN condition) to afford (S)-1-((5-(difluoromethyl)-3-fluoro-1H-indazol-7-yl)sulfonyl)-N-(4-(oxetan-3-yl)-3,4-dihydro-2H-benzo[b][1,4]oxazin-7-yl)azetidine-2-carboxamide (184) (0.040 g, 0.074 mmol, 86.3% yield) as a off-white solid.

LCMS of 184: m/z=538.1 [M+H]⁺

¹H-NMR of 184: (CDCl₃, 400 MHz) δ 11.50 (br. S, 1H), 8.19 (s, 1H), 8.07-8.03 (m, 2H), 7.09 (d, J=2.4 Hz, 1H), 7.03 (dd, J₁=2.4 Hz, J₂=8.8 Hz, 1H), 6.83-6.69 (m, 1H), 6.20 (d, J=8.8 Hz, 1H), 4.91-4.86 (m, 2H), 4.83-4.79 (m, 2H), 4.75-4.70 (m, 1H), 4.62-4.54 (m, 1H), 4.40-4.36 (m, 2H), 3.90-3.85 (m, 1H), 3.80-3.75 (m, 1H), 3.23-3.21 (m, 2H), 2.58-2.37 (m, 2H)

The following compound was synthesized in a similar manner as 184 replacing I-18 with the reactant indicated.

| | | LCMS | |
|---|---|---|---|
| Example | Reactant | m/z | ¹H-NMR |
| 185 | I-15 | 566.1 | (DMSO-d₆, 400 MHz) δ 13.75 (br. s, |
| | | | 1H), 10.08 (br. s, 1H) , 8.42 (s, 1H), |
| | | | 8.08 (s, 1H), 7.25 (t, J = 5.6 Hz, 1H), |
| | | | 7.06 (d- J = 2.4 Hz, 1H), 7.00-6.97 (m, |
| | | | 1H), 6.85 (d, J = 9.2 Hz, 1H), 4.97 (t- |
| | | | J = 8.8 Hz, 1H), 4.-6-4.14 (m, 2H), |
| | | | 3.-5-3.86 (m, 4H), 3.-7-3.64 (m, 1H), |
| | | | 3.-7-3.44 (m, 2H), 3.-2-3.20 (m, 2H), |
| | | | 2.-1-2.32 (m, 2H), 1.-1-1.67 (m, 2H), |
| | | | 1.62-1.57 (m, 2H) |

### Synthesis of (S)-1-((3-fluoro-5-formyl-1H-indazol-7-yl)sulfonyl)-N-(1-methyl-2-oxo-1,2-dihydropyridin-4-yl)azetidine-2-carboxamide (Example 186)

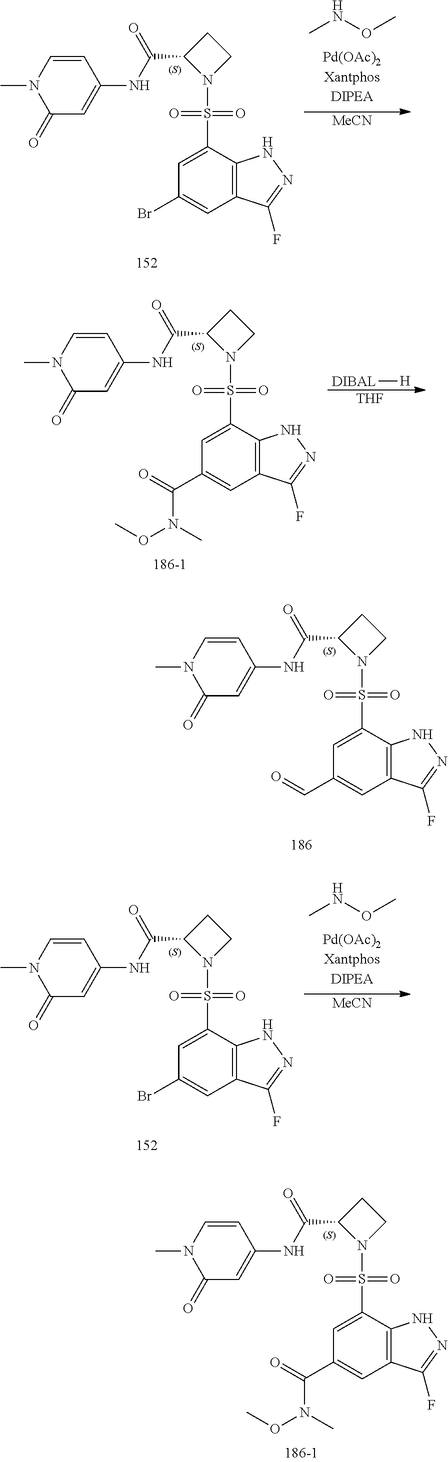

To a solution of 152 (0.500 g, 1.03 mmol) in acetonitrile (10 mL) was added with N, O-dimethylhydroxylamine (0.200 g, 2.06 mmol) followed by N-ethyl-N-isopropylpropan-2-amine (3.10 mmol, 0.539 mL), Pd(OAc)₂ (0.011 g, 0.052 mmol) and Xantphos (0.060 g, 0.100 mmol). The mixture was degassed and purged with CO several times. The mixture was stirred under CO (50 psi) at 80° C. for 12 hours. The reaction mixture was filtered and the filtrate was concentrated under reduce pressure. The residue was purified by reversed-phase flash (0.1% NH₃·H₂O/MeCN) and lyophilized to afford (S)-3-fluoro-N-methoxy-N-methyl-7-((2-((1-methyl-2-oxo-1,2-dihydr282ydroxyam-4-yl)carbamoyl)azetidin-1-yl)sulfonyl)-1H-indazole-5-carboxamide (186-1) (0.200 g, 0.410 mmol, 39.0% yield) as a yellow solid.

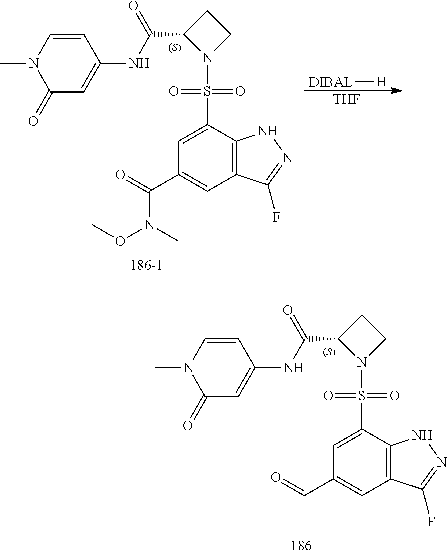

To a solution of 186-1 (0.200 g, 0.410 mmol) in tetrahydrofuran (10 mL) was added DIBAL-H (1 M, 2.03 mL) drop wise at −70° C. under nitrogen atmosphere. The reaction mixture was stirred at −70° C. for 1 hour then quenched with methanol (5 mL) at −70° C. and the reaction mixture was stirred at −70° C. for 0.5 hour. The reaction mixture was concentrated under reduced pressure. The residue was dissolved in methanol (200 mL), filtered, and the filtrate was concentrated under reduced pressure. The residue was purified by column chromatography (SiO₂, dichloromethane:methanol=10/1) to afford (S)-1-((3-fluoro-5-formyl-1H-indazol-7-yl)sulfonyl)-N-(1-methyl-2-oxo-1,2-dihydropyridin-4-yl)azetidine-2-carboxamide (186) (0.120 g, 60.0% yield) as a yellow solid.

LCMS of 186: m/z 434.3 [M+H]⁺

¹H-NMR of 186: (DMSO-d₆, 400 MHz) δ 13.49 (br. s, 1H), 10.19 (s, 1H), 10.11 (s, 1H), 8.79 (s, 1H), 8.32 (s, 1H), 7.61 (d, J=7.6 Hz, 1H), 6.70 (d, J=2.0 Hz, 1H), 6.32 (dd, J₁₌2.4 Hz, J₂=7.2 Hz, 1H), 4.80 (t-J=8.0 Hz, 1H), 3.89-3.77 (m, —H), 3.35 (s, 3H), 2.34-2.26 (m, 2H)

### Synthesis of (S)-1-((2-cyano-5-methyl-1H-indol-7-yl)sulfonyl)-N-(1-methyl-2-oxo-1,2-dihydropyridin-4-yl)azetidine-2-carboxamide (Example 187)

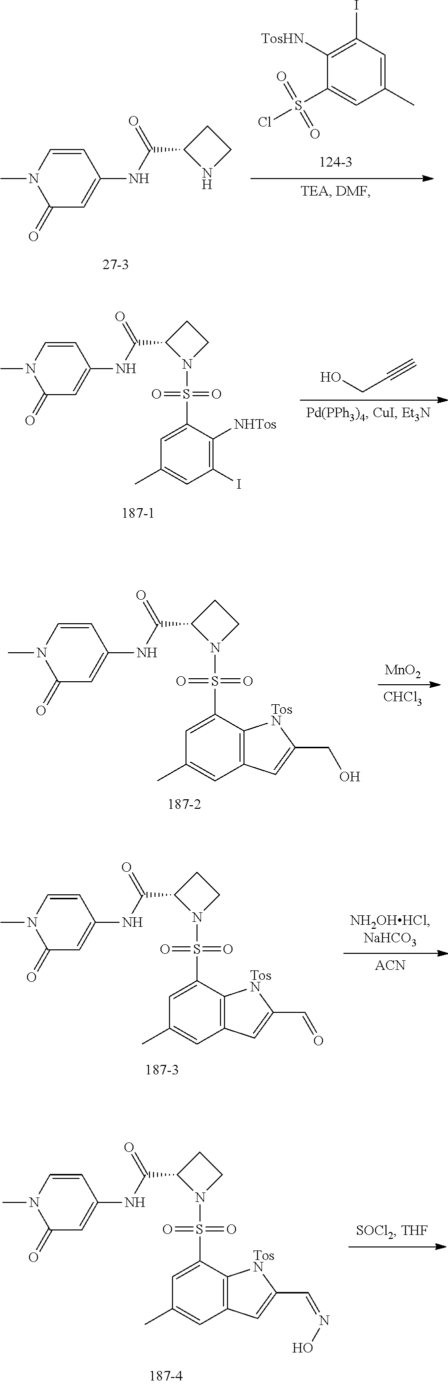

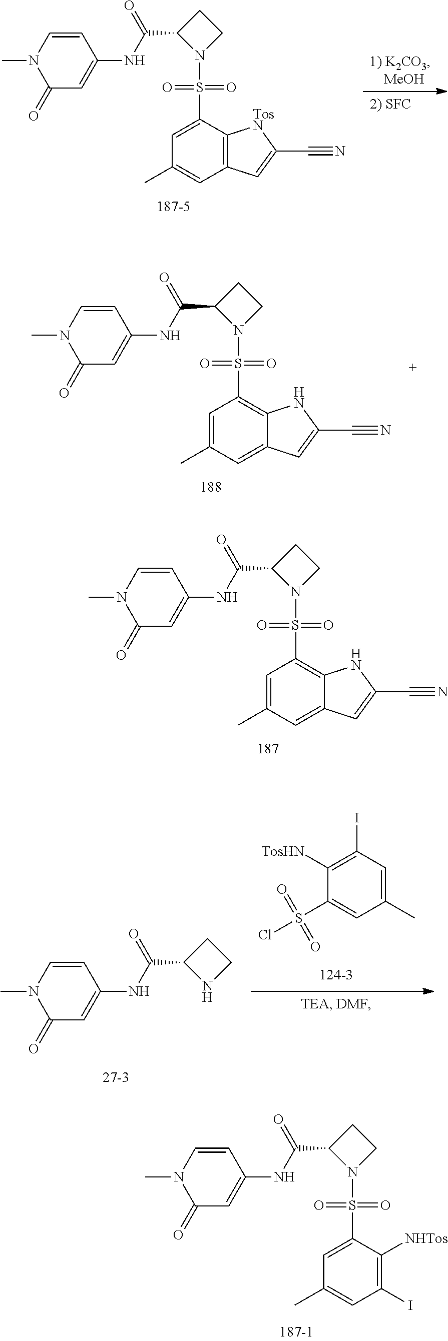

To a solution of 27-3 as the trifluoroacetic acid salt (0.650 g, crude) in dimethylformamide (10 mL) was added triethylamine (1.41 mL, 10.1 mmol) at 0° C. The mixture was stirred at 0° C. for 5 minutes then a solution of 124-3 (1.47 g, 3.04 mmol) in dichloromethane (4 mL) was added to the mixture and the mixture was stirred at 25° C. for 60 minutes. The mixture was poured into water (100 mL) and extracted with ethyl acetate (50 mL*3). The combined organic layers were washed with brine (100 mL), dried over anhydrous sodium sulfate, filtered, and the filtrate was concentrated under reduced pressure. The crude product was purified by reversed-phase flash (0.1% TFA/MeCN condition) to afford (S)-1-((3-iodo-5-methyl-2-((4-methylphenyl)sulfonamido)phenyl)sulfonyl)-N-(1-methyl-2-oxo-1,2-dihydropyridin-4-yl)azetidine-2-carboxamide (187-1) (0.900 g, 66.3% yield) as a yellow solid.

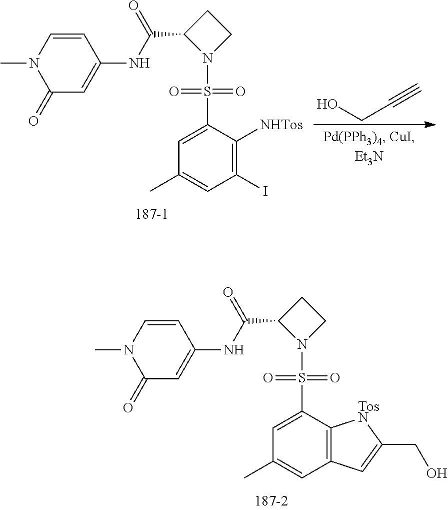

To a mixture of 187-2 (0.700 g, 1.07 mmol), CuI (0.023 g, 0.106 mmol), trimethylamine (0.445 mL, 3.20 mmol) and prop-2-yn-1-ol (1.21 mL, 20.5 mmol) in dimethylformamide (7 mL) was added Pd(PPh₃)₄ (0.123 g, 0.107 mmol) a in one portion at 25° C. under nitrogen. The mixture was stirred at 90° C. for 20 hours. The reaction mixture added into water (150 mL) and extracted with n-butylalcohol (80 mL*3). The combined organic layers were washed with brine (50 mL*3), dried over anhydrous sodium sulfate, filtered and the filtrate was concentrated under reduced pressure. The residue was purified by column chromatography (SiO₂, petroleum ether:ethyl acetate=1:1˜dichloromethane:methanol=20:1) to afford (S)-1-((2-(hydroxymethyl)-5-methyl-1-tosyl-1H-indol-7-yl)sulfonyl)-N-(1-methyl-2-oxo-1,2-dihydropyridin-4-yl)azetidine-2-carboxamide (187-2) (0.320 g, 32.4% yield) as a brown oil.

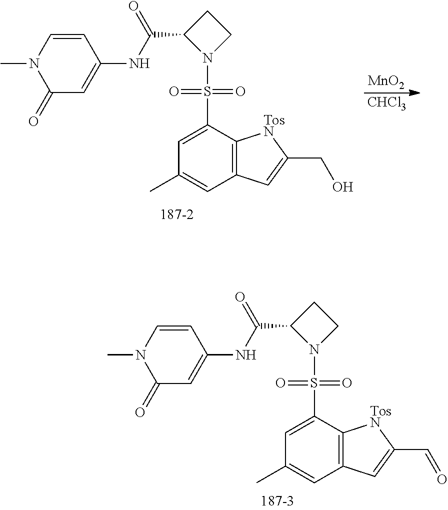

To a mixture of 187-2 (0.220 g, 0.304 mmol) in trichloromethane (5 mL) was added manganese(IV)oxide (0.793 g, 9.12 mmol) at 25° C. The result mixture was stirred at 80° C. for 12 hours. The mixture was added methanol (50 mL), combined with another batch (0.100 g scale) and filtered. The filtrate was concentrated under reduced pressure to afford (S)-1-((2-formyl-5-methyl-1-tosyl-1H-indol-7-yl)sulfonyl)-N-(1-methyl-2-oxo-1,2-dihydropyridin-4-yl)azetidine-2-carboxamide (187-3) (0.250 g, 0.414 mmol, 93.9% yield) as a yellow solid. No further purification was performed.

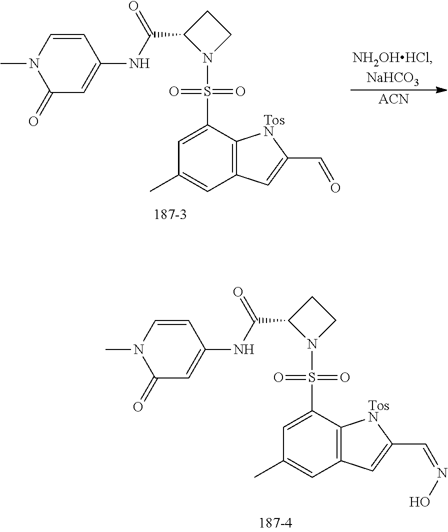

To a solution of 187-3 (0.250 g, 0.414 mmol) and hydroxylamine hydrochloride (0.144 g, 2.07 mmol) in acetonitrile (3 mL) was added sodium bicarbonate (0.174 g, 2.07 mmol) under nitrogen. The mixture was stirred at 80° C. for 2 hours. The mixture was diluted with water (40 mL), extracted with ethyl acetate (30 mL*3). The combined organic layers were washed with brine (30 mL), dried over anhydrous sodium sulfate, filtered, and the filtrate was concentrated under reduced pressure 285ydroxyamino,Z)-1-((2-((hydroxyimino)methyl)-5-methyl-1-tosyl-1H-indol-7-yl)sulfonyl)-N-(1-methyl-2-oxo-1,2-dihydropyridin-4-yl)azetidine-2-carboxamide (187-4) (0.220 g, 83.2% yield) as a yellow solid. No further purification was performed.

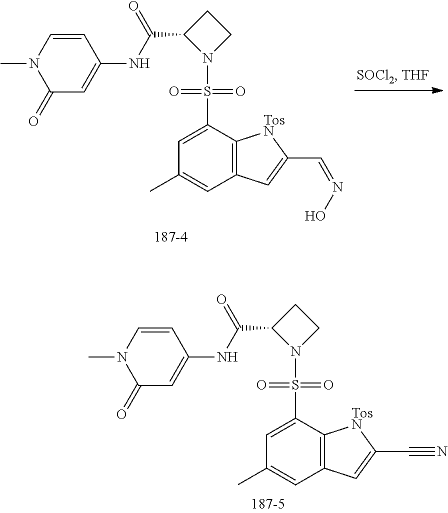

To a solution of 187-4 (0.220 g, 0.345 mmol) in tetrahydrofuran (5 mL) was added thionyl chloride (0.050 mL, 0.689 mmol) under nitrogen. The mixture was stirred at 25° C. for 1.5 hours then concentrated under reduced pressure to afford (S)-1-((2-cyano-5-methyl-1-tosyl-1H-indol-7-yl)sulfonyl)-N-(1-methyl-2-oxo-1,2-dihydropyridin-4-yl)azetidine-2-carboxamide (187-5) (0.180 g, 87.7% yield) as a yellow solid. No further puriufication was performed.

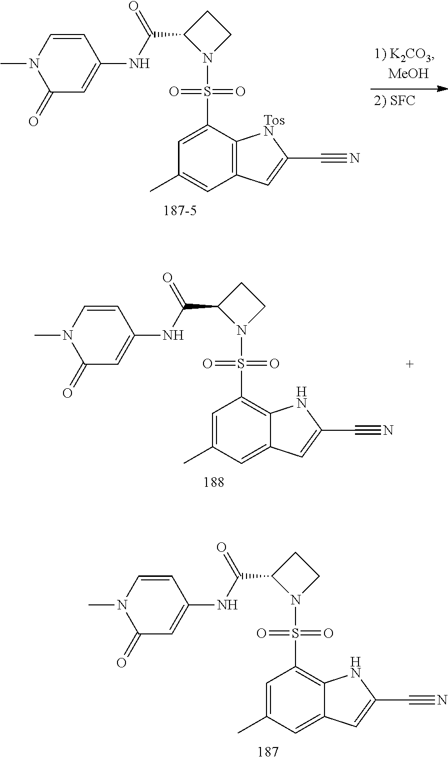

To a 187-5 (0.180 g, 0.302 mmol) in methanol (2.0 mL) was added potassium carbonate (0.209 g, 1.51 mmol) at 25° C. The mixture was stirred at 25° C. for 1 hour then diluted with water (30 mL) and extracted with ethyl acetate (20 mL*3). The combined organic layers were washed with brine (50 mL), dried over anhydrous sodium sulfate, filtered, and the filtrate was concentrated under reduced pressure. The residue was purified by prep-HPLC (column: Phenomenex Synergi C₁₈ 150*25 mm*10 um; mobile phase: [water (0.1% TFA)-ACN]; B %: 35%-65%, 10 min) and further purified by SFC separation (column: Daicel ChiralPak IG (250*30 mm, 10 um); mobile phase: [0.1% NH₃H₂O MEOH]; B %: 70%-70%, 8.2; 60 min) to afford (R)-1-((2-cyano-5-methyl-1H-indol-7-yl)sulfonyl)-N-(1-methyl-2-oxo-1,2-dihydropyridin-4-yl)azetidine-2-carboxamide (188) (0.007 g, 5.68% yield) and (S)-1-((2-cyano-5-methyl-1H-indol-7-yl)sulfonyl)-N-(1-methyl-2-oxo-1,2-dihydropyridin-4-yl)azetidine-2-carboxamide (187) (0.061 g, 47.3% yield) as a white solid.

LCMS of 187: m/z 426.0 [M+H]⁺.

¹H-NMR of 187: (CD₃OD, 400 MHz): δ 7.83 (s, 1H), 7.72 (s, 1H), 7.61 (d, J=7.6 Hz, 1H), 7.31 (s, 1H), 6.89 (d, J=2.4 Hz, 1H), 6.74 (dd, J₁=2.4 Hz, J₂=7.2 Hz, 1H), 4.79 (t-J=8.4 Hz, 1H), 3.-2-3.86 (m, 1H), 3.71-3.66 (m, 1H), 3.53 (s, —H), 2.51 (s, 3H), 2.42-2.36 (m, 2H)

LCMS of 188: m/z 426.0 [M+H]⁺.

¹H-NMR of 188: (CD₃OD, 400 MHz): δ 7.83 (s, 1H), 7.73 (s, 1H), 7.61 (d, J=7.6 Hz, 1H), 7.31 (s, 1H), 6.89 (d, J=2.4 Hz, 1H), 6.74 (dd, J₁=2.4 Hz, J₂=7.2 Hz, 1H), 4.79 (t-J=8.8 Hz, 1H), 3.-2-3.86 (m, 1H), 3.71-3.66 (m, 1H), 3.53 (s, —H), 2.51 (s, 3H), 2.42-2.36 (m, 2H)

The following compound was synthesized in a similar manner as 187 replacing 27-3 with the reactant indicated.

| | | LCMS | |
|---|---|---|---|
| Example | Reactant | m/z | ¹H-NMR |
| 195 | I-49 | 514.1 | (CD₃OD, 400 MHz) δ = 7.82 (s, 1H), |
| | | | 7.72 (d, J = 1.2 Hz, 1H), 7.69 (d- J = |
| | | | 2.4 Hz, 1H), 7.51-7.45 (m, 1H), 7.30 |
| | | | (s, 1H), 7.13 (d- J = 8.8 Hz, 1H), 4.82- |
| | | | 4.78 (m, 1H), 3.92 (q- J = 6.8 Hz, 1H), |
| | | | 3.86-3.83 (m, 4H), 3.69 (q- J = 6.8 Hz, |
| | | | 1H), 3.05-3.01 (m, -H), 2.50 (s, 3H), |
| | | | 2.44-2.36 (m, 2H) |
| 199 | I-52 | 516.1 | (CD₃OD, 400 MHz) δ 7.83 (s, 1H), |
| | | | 7.73 (s, 1H), 7.67 (d, J = 2.4 Hz, 1H), |
| | | | 7.47 (dd, J₁ = 2.4 Hz, J₂ = 8.8 Hz, 1H), |
| | | | 7.30 (s, 1H), 7.07 (d- J = 8.8 Hz, 1H), |
| | | | 4.82-4.76 (m, 1H), 4.18 (t- J = 5.6 Hz, |
| | | | 2H), 3.-6-3.88 (m, 1H), 3.72-3.66 (m, |
| | | | 1H), 2.85 (t, J = 5.6 Hz, -H), 2.50 (s, |
| | | | 3H), 2.43-2.35 (m, 8H) |

### Synthesis of (S)—N-(3-chloro-4-morpholinophenyl)-1-((5-(difluoromethyl)-1H-indazol-7-yl)sulfonyl)azetidine-2-carboxamide (Example 190)

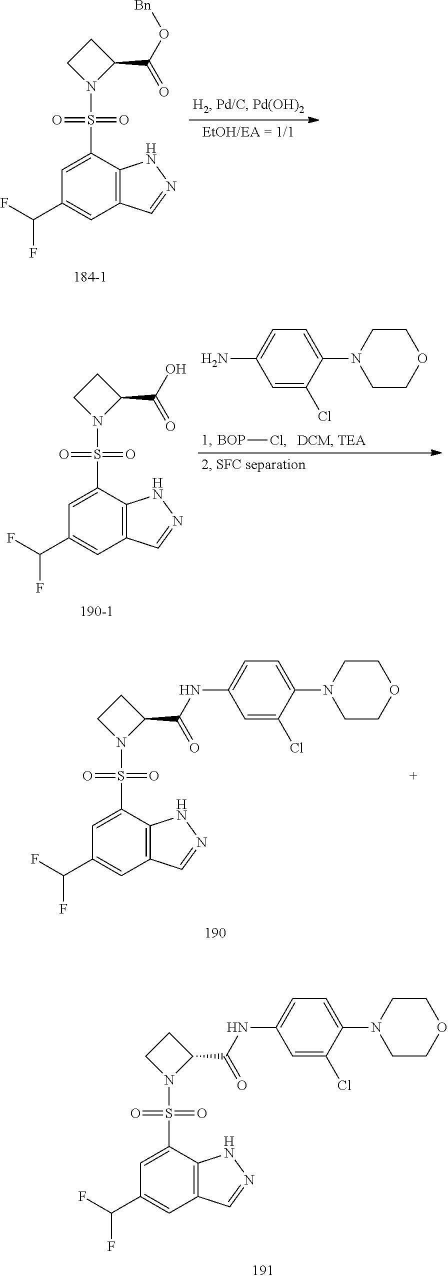

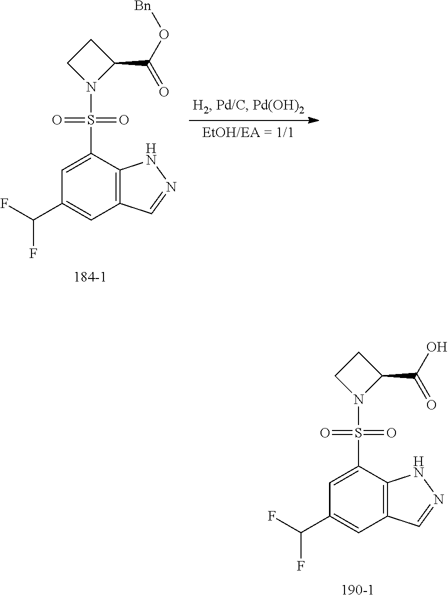 [SMILES: O=C(OCc1ccccc1)[C@@H]1CCN1S(=O)(=O)c1cc(C(F)F)cc2cn[nH]c12 ; O=C(O)[C@@H]1CCN1S(=O)(=O)c1cc(C(F)F)cc2cn[nH]c12]

To a solution of 184-1 (0.150 g, 0.356 mmol) in a mixture of ethyl acetate (10 mL) and methanol (10 mL) was added Pd(OH)₂ (0.010 g, 0.007 mmol, 10% on charcoal, wet) and Pd/C (0.010 g, 0.356 mmol, 10% on charcoal, wet) under nitrogen atmosphere. The mixture was degassed and purged with hydrogen three times. The mixture was stirred at 25° C. under hydrogen atmosphere (15 psi) for 2 hours. The mixture was diluted with methanol (30 mL), filtered, and the filtrate was concentrated under reduced pressure to afford (S)-1-((5-(difluoromethyl)-1H-indazol-7-yl)sulfonyl)azetidine-2-carboxylic acid (190-1) (0.140 g, crude) as a white solid.

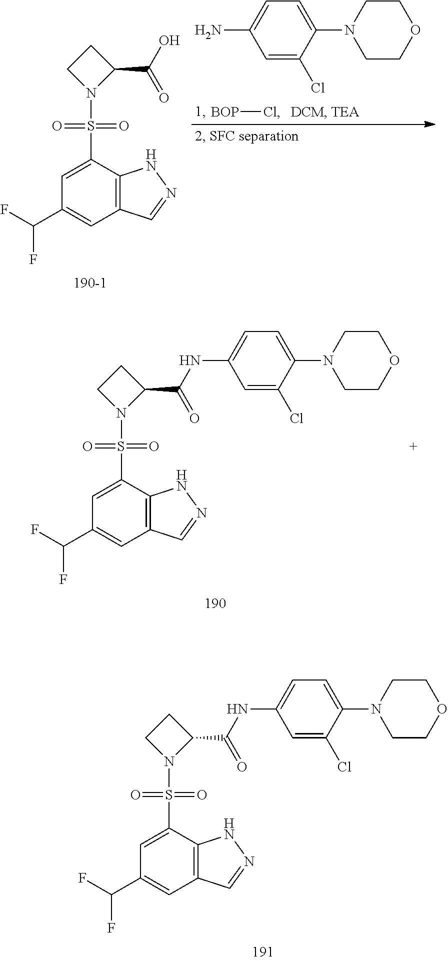

To a solution of 190-1 (0.100 g, 0.302 mmol) in dichloromethane (1 mL) was added triethylamine (0.126 mL, 0.906 mmol) and BOP-Cl (0.125 g, 0.491 mmol), followed by 3-chloro-4-morpholinoaniline (0.064 g, 0.302 mmol). The mixture was stirred at 25° C. for 12 hours then diluted with water (5 mL) and extracted with dichloromethane (5 mL*3). The combined organic layers were washed with brine (10 mL*3), dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (0.05% ammonia hydroxide v/v)-ACN]; B %: 28%-58%, 7 min) and further separated by SFC (column: DAICEL CHIRALCEL OJ (250 mm*30 mm, 10 um); mobile phase: [0.1% NH₃H₂O MEOH]; B %: 60%-60%, 4.3 min; 30 min) to afford (S)—N-(3-chloro-4-morpholinophenyl)-1-((5-(difluoromethyl)-1H-indazol-7-yl) sulfonyl) azetidine-2-carboxamide (190) (0.039 g, 24.0% yield) as a white solid. And also (2R)—N-(3-chloro-4-morpholino-phenyl)-1-[[5-(difluoromethyl)-1H-indazol-7-yl]sulfonyl]azetidine-2-carboxamide (191) (0.021 g, 12.4% yield) was obtained as a white solid.

LCMS of 190: m/z: 526.1[M+H]⁺

¹H-NMR of 190: (DMSO-d₆, 400 MHz) δ 14.00 (br. s, 1H), −0.26 (br. s, 1H), 8.67-8.36 (m, 2H), 8.00 (s, —H), 7.78 (s, 1H), 7.-3-7.42 (m, 1H), 7.40-7.12 (m, 2H), 4.88 (t-J=8.4 Hz, 1H), 3.89-3.83 (m, 1H), 3.74 (t-J=4.8 Hz, 4H), 3.71-3.65 (m, 1H), 2.94 (t-J=4.4 Hz, 4H), 2.39-2.31 (m, 2H)

LCMS of 191: m/z: 526.2[M+H]⁺

¹H-NMR of 191: (DMSO-d₆, 400 MHz) δ 14.00 (br. s, 1H), 10.29 (br. s, 1H), 8.47 (d, J=7.2 Hz, 2H), 8.00 (s, 1H), 7.79 (s, 1H), 7.48 (d-J=8.4 Hz, 1H), 7.40-7.11 (m, 2H), 4.88 (t-J=8.4 Hz, 1H), 3.89-3.83 (m, 1H), 3.74 (t-J=4.4 Hz, 4H), 3.71-3.66 (m, 1H), 2.94 (t-J=4.4 Hz, 4H), 2.38-2.30 (m, 2H)

### Synthesis of N-(3-chloro-4-(1-(oxetan-3-yl)piperidin-4-yl)phenyl)-2-((N,5-dimethyl-1H-indazole)-7-sulfonamido)acetamide (Example 193)

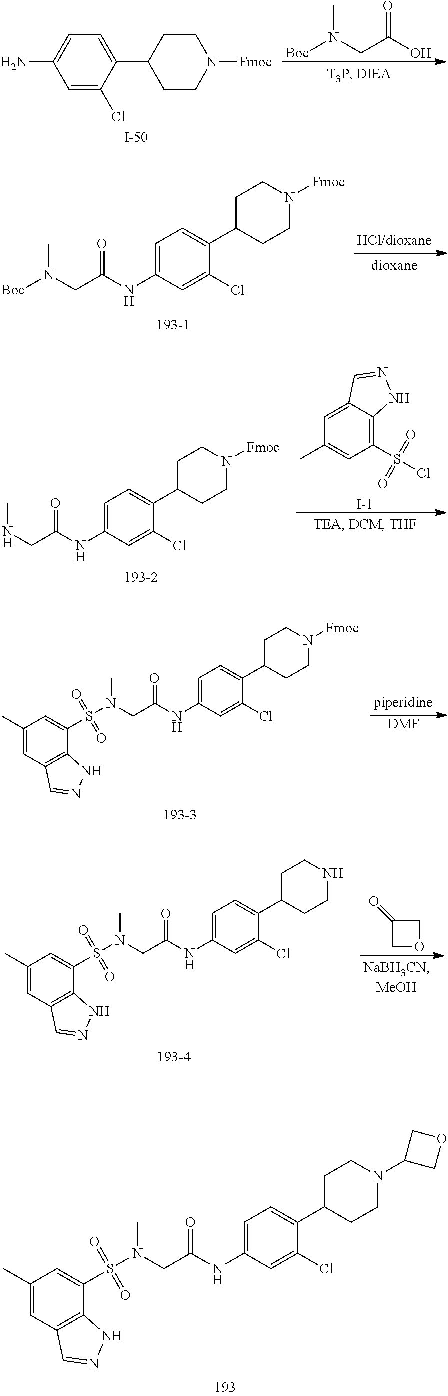

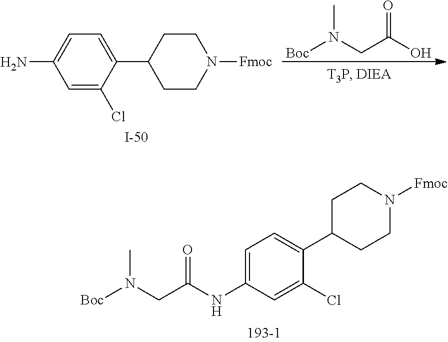

To a solution I-50 (0.700 g, 1.13 mmol) in dichloromethane (14 mL) was added N-(tert-butoxycarbonyl)-N-methylglycine (0.260 g, 1.35 mmol) followed by diisopropylethylamine (0.440 g, 3.38 mmol) and T₃P (1.08 g, 1.69 mmol, 50% w/w in ethyl acetate). The above reaction solution was stirred at 20° C. for 1 hour then quenched with water (50 mL) and extracted with dichloromethane (30 mL*3). The combined organic layers were washed with brine (100 mL), dried over anhydrous sodium sulfate, filtered, and the filtrate was concentrated under reduced pressure. The residue was purified by silica gel column (petroleum ether:ethyl acetate=1:0 to 1:1) to afford (9H-fluoren-9-yl)methyl 4-(4-(2-((tert-butoxycarbonyl)(methyl)amino)acetamido)-2-chlorophenyl)piperidine-1-carboxylate (193-1) (0.500 g, 73.4% yield) as a white solid.

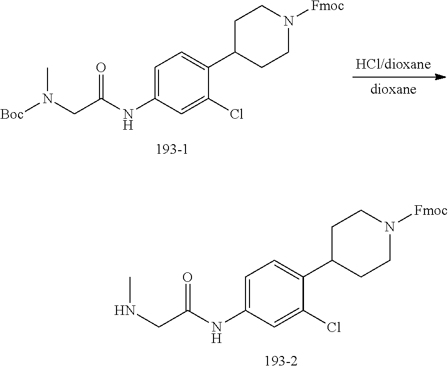

To a solution of 193-1 (0.560 g, 0.930 mmol) in dioxane (4 mL) was added a solution of HCl in dioxane (4 M, 4 mL, 16 mmol). The mixture was stirred at 20° C. for 1 hour then concentrated under reduced pressure to afford (9H-fluoren-9-yl)methyl 4-(2-chloro-4-(2-(methylamino)acetamido)phenyl)piperidine-1-carboxylate (193-2) hydrochloride salt (0.500 g, 99.8% yield) as a white solid. No further purification was performed.

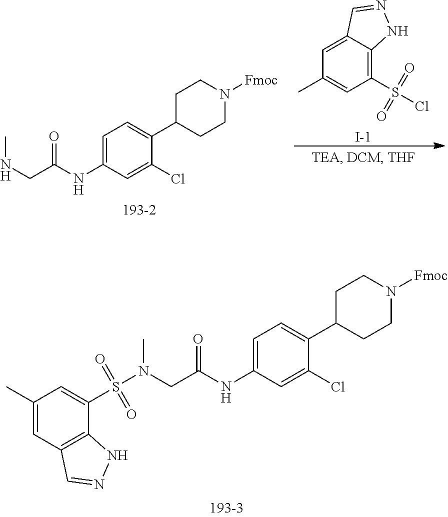

To a solution of 193-2 hydrochloride salt (0.420 g, 0.780 mmol) in dichloromethane (8 mL) was added triethylamine (0.390 g, 3.89 mmol). To this was added a solution of I-1 (0.250 g, 1.09 mmol) in tetrahydrofuran (4 mL) drop wise at 20° C. The mixture was concentrated under reduced pressure and the residue purified by reversed-phase flash (0.1% TFA/MeCN condition), then concentrated under reduced pressure to afford (9H-fluoren-9-yl)methyl 4-(2-chloro-4-(2-((N,5-dimethyl-1H-indazole)-7-sulfonamido)acetamido)phenyl)piperidine-1-carboxylate (193-3) (0.400 g, 73.7% yield) as a white solid.

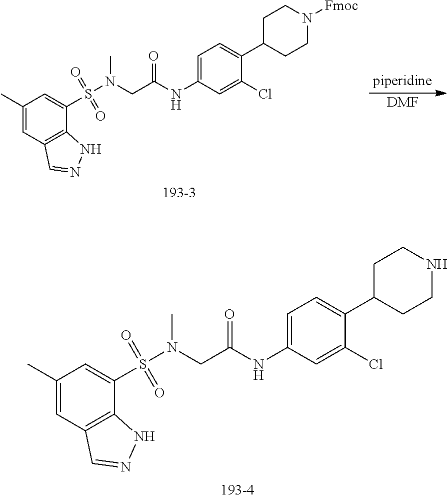

A solution of 193-3 (0.400 g, 0.570 mmol) in a mixture of dimethylformamide (3 mL) and piperidine (1 mL) was stirred at 20° C. for 0.5 hour. The mixture was concentrated under reduced pressure and the residue was purified by prep-HPLC (column: Phenomenex luna C₁₈ 150*40 mm*15 um; mobile phase: [water (0.1% TFA)-ACN]; B %: 17%-47%, 11 min) and the eluent removed by lyophilization to afford N-(3-chloro-4-(piperidin-4-yl)phenyl)-2-((N,5-dimethyl-1H-indazole)-7-sulfonamido)acetamide (193-4) as the trifluoroacetic salt (0.250 g, 66.3% yield) as a white solid.

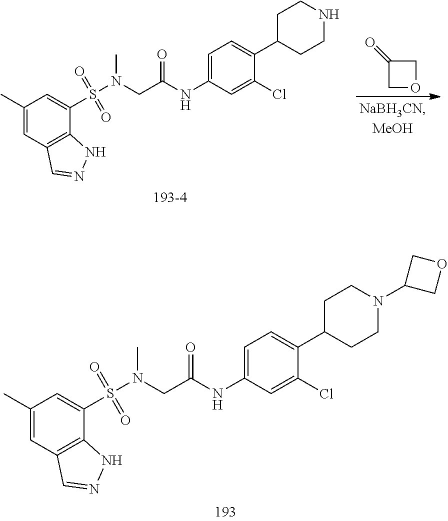 [SMILES: Cc1cc(S(=O)(=O)N(C)CC(=O)Nc2ccc(C3CCNCC3)c(Cl)c2)c2[nH]ncc2c1 ; O=C1COC1 ; Cc1cc(S(=O)(=O)N(C)CC(=O)Nc2ccc(C3CCN(C4COC4)CC3)c(Cl)c2)c2[nH]ncc2c1]

To a solution of 193-4 as the trifluoroacetic salt (0.100 g, 0.170 mmol) in methanol (2 mL) was added potassium acetate (0.020 g, 0.200 mmol) followed by oxetan-3-one (0.250 g, 3.39 mmol). The mixture was stirred at 50° C. for 2 hours then sodium cyanoborohydride (0.050 g, 0.850 mmol) was added at 20° C. and stirred at 20° C. for an additional 2 hours. Water (50 mL) was added and the mixture extracted with dichloromethane (50 mL*2). The combined organic layers were dried over anhydrous sodium sulfate, filtered, and the filtrate was concentrated under reduced pressure. The residue was purified by prep-HPLC (column: Phenomenex Gemini-NX C₁₈ 75*30 mm*3 um; mobile phase: [water (0.05% ammonia hydroxide v/v)-ACN]; B %: 26%-56%, 7 min), then the solvent was removed by lyophilization to afford N-(3-chloro-4-(1-(oxetan-3-yl)piperidin-4-yl)phenyl)-2-((N,5-dimethyl-1H-indazole)-7-sulfonamido)acetamide (193) (0.040 g, 47% yield) as a white solid.

LCMS of 193: m/z 532.4 [M+H]⁺

¹H-NMR of 193: (DMSO-d₆, 400 MHz): δ=13.22 (br. s, 1H), 10.17 (br. s, 1H), 8.19 (s, 1H) 7.90 (s, 1H), 7.68 (d, J=1.6 Hz, 1H), 7.62 (d-J=1.2 Hz, 1H), 7.38-7.32 (m, 2H), 4.54 (t, J=6.4 Hz, 2H), 4.44 (t, J=6.0 Hz, —H), 4.08 (s, 2H), 3.-4-3.39 (m, 1H), 2.85-2.77 (m, 6H), 2.44 (s, 3H), 1.87 (dt, J₁=2.4 Hz, −J₂=9.2 Hz, 2H), 1.73-1.60 (m, 4H)

The following compound was synthesized in a similar manner to 193 replacing oxetan-3-one in the conversion of 193-4 to 193 with the reactant indicated.

| | | LCMS | |
|---|---|---|---|
| Example | Reactant | m/z | ¹H-NMR |
| 194 | paraform- | 490.2 | (DMSO-d₆, 400 MHz): δ = 13.21 (br. s, |
| | aldehyde | | 1H), 10.16 (br. s, 1H), 8.19 (s, 1H), |
| | | | 7.90 (s, 1H), 7.68 (d, J = 2.0 Hz, 1H), |
| | | | 7.62 (d- J = 0.8 Hz, 1H), 7.39-7.26 (m, |
| | | | -H), 4.08 (s, 2H), 2.90-2.76 (m, 6H), |
| | | | 2.44 (s, -H), 2.19 (s, 3H), 2.-2-1.90 (m, |
| | | | 2H), 1.71-1.58 (m, 4H) |

The following compound was synthesized in a similar manner as 193 replacing N-(tert-butoxycarbonyl)-N-methylglycine in conversion of I-50 to 193-1 with the reactant indicated.

| Ex- | | LCMS | |
|---|---|---|---|
| ample | Reactant | m/z | ¹H-NMR |
| 196 | 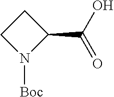 [SMILES: CC(C)(C)OC(=O)N1CC[C@H]1C(=O)O] (S)-1-(tert- butoxycarbonyl)azetidine- 2-carboxylic acid | 544.2 | (CD₃OD, 400 MHz) δ 8.16 (s, 1-), 7.94 (s, 1 H), 7.80-7.75 (m, 2H), 7.43 (dd, J1 = 2.4 Hz, J2 = 8.4 Hz, 1H), 7.33 (d, J = 8.4 Hz, 1H), 4.82 (t, J = 8.4 Hz, 1H), 4.72 (t, J = 6.8 Hz, 2H), 4.64 (t-J = 6.4 Hz, 2H), 3.-7- 3.88 (m, 1H), 3.-3-3.66 (m, 1H), 3.-0-3.52 (m, 1H), 3.-1-3.01 (m, 1H), 2.98- |
| | | | 2.91 (m,-H), 2.53 (s, 3H), |
| | | | 2.44-2.35 (m, 2H), 2-06- |
| | | | 1.98 (m, 2H), 1.-0-1.84 |
| | | | (m, 2H), 1.83-1.71 (m, 2H) |

The following compound was synthesized in a similar manner as 196 replacing oxetan-3-one in the final step with the reactant indicated.

| | | LCMS | |
|---|---|---|---|
| Example | Reactant | m/z | ¹H-NMR |
| 197 | paraform- | 502.2 | CD₃OD, 400 MHz) δ 8.16 (s, 1H), 7.94 |
| | aldehyde | | (s, 1H), 7.78 (s, 2H), 7.42 (dd, J₁ = 2.4 |
| | | | Hz, J₂ = 8.4 Hz, 1H), 7.32 (d, J = 8.4 |
| | | | Hz, 1H), 4.82 (t- J = 8.4 Hz, 1H), 3.-5- |
| | | | 3.89 (m, 1H), 3.-4-3.63 (m, 1H), 3.08- |
| | | | 3.01 (m, -H), 2.53 (s, 3H), 2.44-2.38 |
| | | | (m, -H), 2.36 (s, 3H), 2.-6-2.18 (m, |
| | | | 2H), 1.-9-1.83 (m, 2H), 1.82-1.73 (m, |
| | | | 2H) |

### Synthesis of (S)-1-((2-cyano-5-methyl-1H-indol-7-yl)sulfonyl)-N-(4-(oxetan-3-yl)-3,4-dihydro-2H-benzo[b][1,4]oxazin-7-yl)azetidine-2-carboxamide (Example 206)

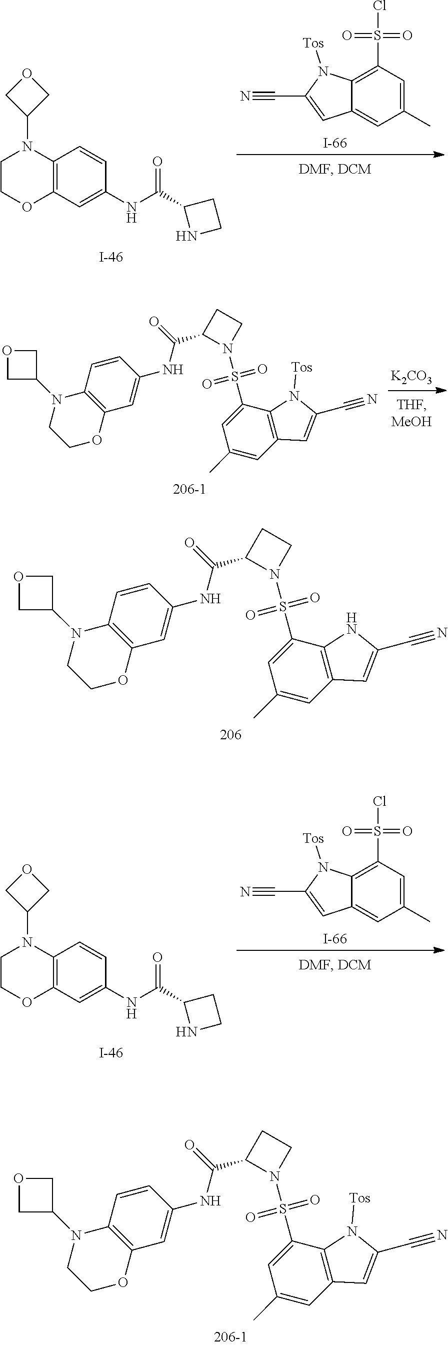

To a solution of I-46 (0.090 g, 0.311 mmol) and triethylamine (0.217 mL, 1.56 mmol) in dimethylformamide (0.5 mL) was added a solution of I-66 (0.127 g, 0.311 mmol) in dimethylformamide (0.5 mL) at 0° C. The mixture was stirred at 25° C. for 1 hour. The mixture was added into water (30 mL) causing a precipitate to form. The solid was collected by filtration and dried under reduced pressure to afford (S)-1-((2-cyano-5-methyl-1-tosyl-1H-indol-7-yl)sulfonyl)-N-(4-(oxetan-3-yl)-3,4-dihydro-2H-benzo[b][1,4]oxazin-7-yl)azetidine-2-carboxamide (206-1) (0.190 g, 71.1% yield) as a white solid.

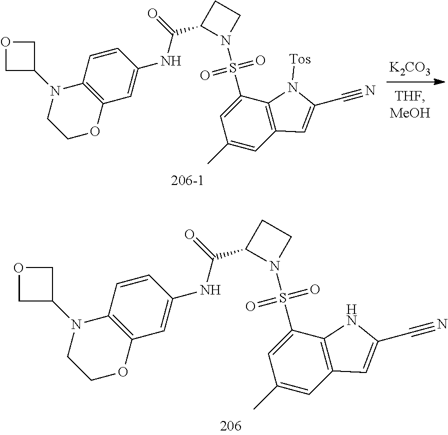

To a solution of 206-1 (0.220 g, 0.231 mmol) in a mixture of methanol (2.5 mL) and tetrahydrofuran (2.5 mL) was added potassium carbonate (0.064 g, 0.462 mmol) at 25° C. The mixture was stirred at 25° C. for 2 hours then poured into water (50 mL) and extracted with ethyl acetate (30 mL*3). The combined organic layers were washed with brine (100 mL), dried over anhydrous sodium sulfate, filtered, and the filtrate was concentrated under reduced pressure. The residue was purified by prep-HPLC (column: Waters Xbridge 150*25 mm*5 um; mobile phase: [water (0.05% ammonia hydroxide v/v)-ACN]; B %: 31%-61%, 9 min) to afford (S)-1-((2-cyano-5-methyl-1H-indol-7-yl)sulfonyl)-N-(4-(oxetan-3-yl)-3,4-dihydro-2H-benzo[b][1,4]oxazin-7-yl)azetidine-2-carboxamide (206) (0.046 g, 38.8% yield) as a white solid.

LCMS of 206: m/z 508.1 [M+H]⁺

¹H-NMR of 206: (DMSO-d₆, 400 MHz): δ 12.93 (br, s, 1H), 9.97 (br, s, 1H), 7.87 (s, 1H), 7.68 (s, 1H), 7.51 (s, 1H), 7.13 (d, J=2.4 Hz, 1H), 6.96 (dd, J₁=2.0 Hz, J₂=8.8 Hz, 1H), 6.29 (d, J=8.8 Hz, 1H), 4.84 (t, J=8.4 Hz, 1H), 4.77 (t, J=6.4 Hz, 2H), 4.66 (t-J=6.4 Hz, 2H), 4.59-4.51 (m, 1H), 4.30 (t-J=4.0 Hz, 2H), 3.-3-3.75 (m, 1H), 3.63-3.55 (m, 1H), 3.19 (t, J=4.4 Hz, —H), 2.47 (s, 3H), 2.37-2.26 (m, 2H)

The following compound was synthesized in a similar manner as 206 replacing I-46 with the reactant indicated.

| | | LCMS | |
|---|---|---|---|
| Example | Reactant | m/z | ¹H-NMR |
| 207 | I-54 | 496.2 | (DMSO-d6, 400 MHz): δ 1-.03 (br. s, |
| | | | 1H), 10.15-10.06 (m, 1H), 9.68 (br. s, |
| | | | 1H), 7.88 (s, 1H), 7.69 (d, J = 1.2 Hz, |
| | | | 1H), 7.51 (d- J = 2.0 Hz, 1H), 7.-8-7.42 |
| | | | (m, 2H), 7.02-6.96 (m, 1H), 4.89 (t, J = |
| | | | 8.4 Hz, 1H), 4.30 (t- J = 4.4 Hz, 2H), |
| | | | 3.-7-3.80 (m, 1H), 3.-3-3.59 (m, 1H), |
| | | | 3.-6-3.53 (m, 2H), 2.96-2.87 (m, -H), |
| | | | 2.47 (s, 3H), 2.39-2.29 (m, 2H), 2.21 |
| | | | (s, 3H) |
| 208 | I-45 | 496.2 | (DMSO-d₆, 400 MHz) δ 12.45 (br. s, |
| | | | 1H), 9.83 (br. s, 1H), 7.83 (s, 1H), 7.65 |
| | | | (s, 1H), 7.48 (d, J = 1.6 Hz, 1H), 6.76 |
| | | | (dd, J₁ = 2.0 Hz, -J₂ = 8.0 Hz, 1H), |
| | | | 6.67-6.62 (m, 2H), 4.73 (t, J = 7.2 Hz, |
| | | | 2H), 4.66 (t- J = 6.4 Hz, 2H), 4.-8-4.44 |
| | | | (m, 1H), 4.27-4.25 (m, -H), 4.05 (s, |
| | | | 2H), 3.19-3.17 (m, 2H), 2.80 (s, 3H), |
| | | | 2.45 (s, 3H) |
| 209 | I-55 | 508.2 | (DMSO-d6, 400 MHz) δ 13.05 (br. s, |
| | | | 1H), 9.99 (s, 1H), 7.87 (s, 1H), 7.68 (s, |
| | | | 1H), 7.52 (s, 1H), 6.87 (s, 1H), 6.78 |
| | | | (dd, J1 = 1.6 Hz, J2 = 8.4 Hz, 1H), |
| | | | 6.68 (d, J = 8.4 Hz, 1H), 4.89 (-, J = |
| | | | 8.4 Hz, 1H), 4.81-4.77 (t- J = 6.8 Hz, |
| | | | 2H), 4.70-4.67 (t- J = 7.2 Hz, 2H), |
| | | | 4.58-4.55 (m, 1-), 4.27 (s, 2 H), 3.-5- |
| | | | 3.81 (m, 1H), 3.59-3.55 (m, 1H), 3.23 |
| | | | (s, -H), 2.47 (s, 3H), 2.39-2.33 (m, 2H) |
| 210 | I-56 | 294.2 | (DMSO-d6, 400 MHz) δ 12.39 (br. s, |
| | | | 1H), 8.45 (t, J = 5.4 Hz, 1H), 7.82 (s, |
| | | | 1H), 7.61 (s, 1H), 7.48 (d- J = 1.6 Hz, |
| | | | 1H), 3.86-3.79 (m, 4H), 2.72 (s, 3H), |
| | | | 2.46 (s, 3H), 1.77 (t, J = 2.4 Hz, 3H) |
| 218 | I-61 | 421.3 | (CD3OD, 400 MHz): δ 7.79 (s, -H), |
| | | | 7.68 (s, 1H), 7.-2-7.39 (m, 2H), 7.33- |
| | | | 7.31 (m, 3H), 7.28 (s, 1H), 4.22 (s, |
| | | | 2H), 3.93 (s, 2H), 2.82 (s, 3H), 2.51 (s, |
| | | | 3H) |

### Synthesis of N-(3-chloro-4-methoxyphenyl)-2-((2-cyano-N,5-dimethyl-1H-indole)-7-sulfonamido)acetamide (Example 211)

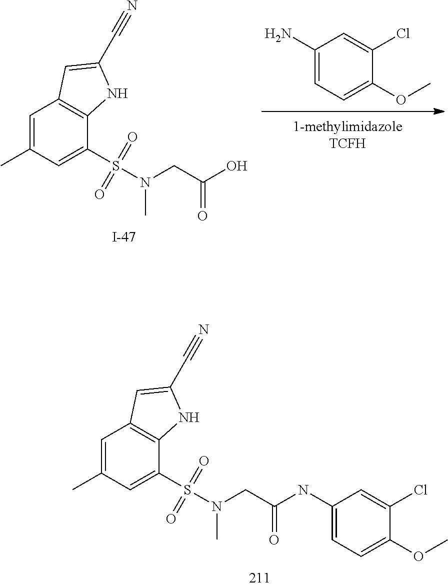

To a solution of I-47 (0.060 g, 0.195 mmol) in acetonitrile (0.6 mL) was added 1-methylimidazole (0.080 g, 0.976 mmol) followed by N,N,N,N-Tetramethylchloroformamidinium hexafluorophosphate (0.110 g, 0.390 mmol). To this was added 3-chloro-4-methoxyaniline (0.046 g, 0.293 mmol) and the mixture was stirred at 40° C. for 1 hour then poured into water (2 mL). the mixture was extracted with ethyl acetate (5 mL*3), dried over anhydrous sodium sulfate, filtered, and the filtrate was concentrated under reduced pressure. The residue was purified by Prep-HPLC (column: Waters xbridge 150*25 mm 10 um; mobile phase: [water (NH₄HCO₃)-ACN]; B %: 36%-66%, 8 min.) to afford N-(3-chloro-4-methoxyphenyl)-2-(2-cyano-N,5-dimethyl-1H-indole-7-sulfonamido)acetamide (211) (0.025 g, 28.0% yield) as a white solid.

LCMS of 211: m/z=447.1[M+H]⁺

¹H-NMR of 211: (CD₃OD, 400 MHz) δ 7.79 (s, 1H), 7.70 (d, J=1.2 Hz, 1H), 7.6 (d, J=2.4, 1H), 7.40 (dd, J₁=4.8 Hz, J₂=9.2 Hz, 1H), 7.28 (s, 1H), 7.10 (d, J=8.8 Hz, 1H), 4.09 (s, 2H), 3.86 (s, 3H), 2.88 (s, 3H), 2.49 (s, 3H)

The following compound was synthesized in a similar manner as 211 using the reactant indicated.

| | | LCMS | |
|---|---|---|---|
| Example | Reactant | m/z | ¹H-NMR |
| 212 | 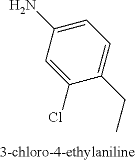 | 445.1 | (DMSO-d₆, 400 MHz) δ 12.28 (br. s, 1H), 10.15 (br. s, 1H), 7.81 (s, 1H), 7.68 (d, J = 1.6 Hz, 1H), 7.62 (s, 1H), 7.47 (d-J = 1.6 Hz, 1H), 7.-3-7.31 (m, 1H), 7.28-7.26 (m, 1H), 4.08 (s, −H), 2.84 (s, 3H), 2.67-2.63 (m, 2H), 2.43 (s, 3H), 1.14 (t, J = 7.6 Hz, 3H) |
| 217 | 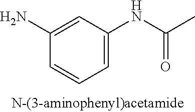 | 440.2 | CD3OD, 400 MHz) δ 7.82 (s, 1H), 7.79 (s, 1H), 7.71 (d-J = 1.6 Hz, 1H), 7.-5- 7.32 (m, 1H), 7.29-7.24 (m, 3H), 4.11 (s, 2H), 2.88 (s, 3H), 2.49 (s, 3H), 2.12 (s, 3H) |

### Synthesis of 2-((2-cyano-N,5-dimethyl-1H-indole)-7-sulfonamido)-N-(2-methoxypyridin-4-yl)acetamide (Example 214)

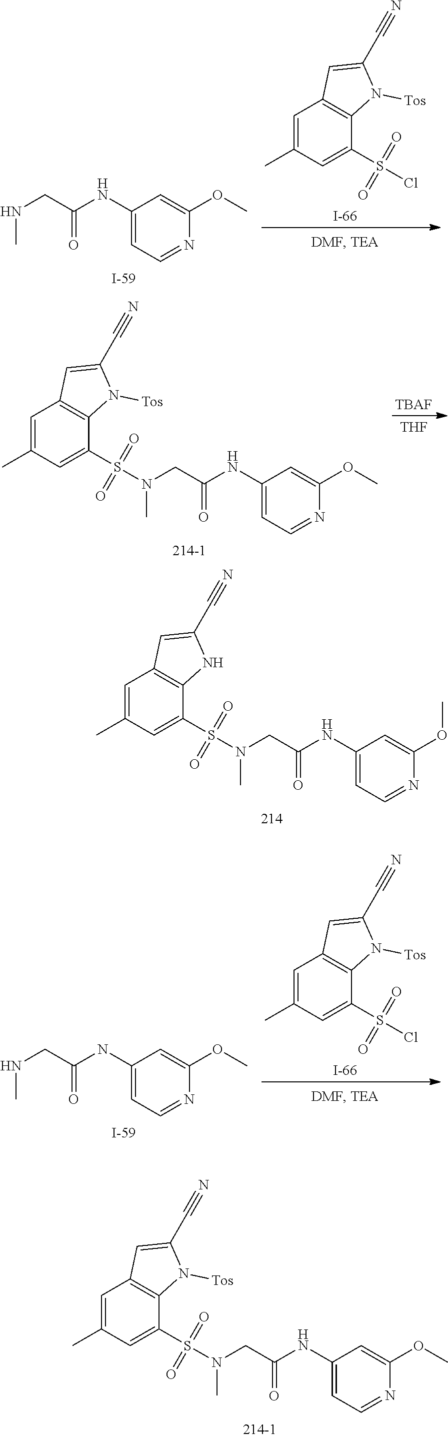

To a solution of I-59 hydrochloric salt (0.052 g, 0.220 mmol) in dimethylformamide (2 mL) was added triethylamine (0.100 g, 0.970 mmol) then I-53 (0.080 g, 0.200 mmol) was added at 0° C. The reaction solution was stirred at 25° C. for 2 hours then diluted with water (20 mL) and extracted with ethyl acetate (20 mL*3). The combined organic layers were washed with brine (15 mL*2), dried over anhydrous sodium sulfate, filtered, and the filtrate was concentrated under reduced pressure. The residue was purified by column chromatography (SiO₂, Petroleum ether/Ethyl acetate=99/1 to 42/58) to afford 2-(2-cyano-N,5-dimethyl-1-tosyl-1H-indole-7-sulfonamido)-N-(2-methoxypyridin-4-yl)acetamide (214-1) (0.090 g, 0.150 mmol) as a brown oil.

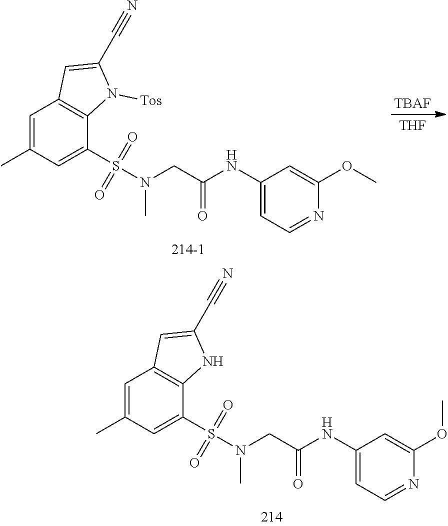

To a solution of 214-1 (0.090 g, 0.160 mmol) in tetrahydrofuran (1.5 mL) was added tetrabutylammoniumfluoride (1 mol/L in tetrahydrofuran, 0.120 g, 0.470 mmol) at 0° C., and the mixture was stirred at 25° C. for 1 hour. The reaction mixture was diluted with water (20 mL) and extracted with ethyl acetate (20 mL*3). The combined organic layers were washed with brine (15 mL*2), dried over anhydrous sodium sulfate, filtered, and the filtrate was concentrated under reduced pressure. The residue was triturated with deionized water (15 mL) at 25° C. for 10 minutes to afford 2-(2-cyano-N,5-dimethyl-1H-indole-7-sulfonamido)-N-(2-methoxypyridin-4-yl)acetamide (214) as a white solid.

LCMS of 214: m/z=414.1 [M+H]⁺

¹H-NMR of 214: (DMSO-d₆, 400 MHz) δ 12.22 (br. s, 1-), 10.37 (s, 1H), 8.03-8.01 (m, 1H), 7.81 (s, 1H), 7.61 (s, —H), 7.46 (s, 1H), 7.02-7.01 (m, 2H), 4.13 (s, 2H), 3.81 (s, 3H), 2.85 (s, 3H), 2.44 (s, 3H)

The following compound was synthesized in a similar manner as 214 replacing I-59 with the reactant indicated.

| | | LCMS | |
|---|---|---|---|
| Example | Reactant | m/z | ¹H-NMR |
| 215 | I-60 | 457.2 | (DMSO-d6 400 MHz) δ 12.28 (br. s, |
| | | | 1H), 10.16 (br. s, 1H), 7.81 (s, 1H), |
| | | | 7.68 (d, J = 2.0 Hz, 1H), 7.61 (d, J = |
| | | | 0.8 Hz, 1H), 7.46 (d- J = 1.6 Hz, 1H), |
| | | | 7.29-7.27 (dd, J1 = 2.0 Hz, J2 = 8.4 |
| | | | Hz, 1H), 6.96 (d, J = 8.4 Hz, 1H), 4.08 |
| | | | (s, 2H), 2.83 (s, -H), 2.43 (s, 3H), 2.-9- |
| | | | 2.02 (m, 1H), 0.-8-0.91 (m, 2H), 0.66- |
| | | | 0.62 (m, 2H) |
| 219 | I-62 | 480.2 | (DMSO-d₆, 400 MHz) δ 12.36 (br. s, |
| | | | 1H), 10.14 (br. s, 1H), 7.81 (s, 1H), |
| | | | 7.63 (s, 1H), 7.54 (s, -H), 7.47 (s, 1H), |
| | | | 7.34-7.28 (m, 2H), 6.97 (d, J = 7.2 Hz, |
| | | | 1H), 4.09 (s, 2H), 3.56 (t, J = 5.2 Hz, |
| | | | 2H), 2.83 (s, 3H), 2.44 (s, 3H), 2.38 (t- |
| | | | J = 6.4 Hz, 2H), 1.85-1.83 (m, 4H) |
| 220 | I-63 | 423.1 | (DMSO-d₆, 400 MHz) δ 12.25 (s, 1H), |
| | | | 10.30 (s, 1H), 8.59 (d, J = 7.2 Hz, 1H), |
| | | | 7.97 (d, J = 2.0 Hz, 1H), 7.91 (d, J = |
| | | | 2.0 Hz, 1H), 7.81 (s, 1H), 7.63 (s, 1H), |
| | | | 7.47 (s, 1H), 6.86 (dd, J₁ = 2.0 Hz, J₂ = |
| | | | 7.6 Hz, 1H), 6.47 (d, J = 1.6 Hz, 1H), |
| | | | 4.14 (s, 2H), 2.87 (s, 3H), 2.44 (s, 3H) |
| 221 | I-13 | | (CDCl₃, 400 MHz) δ 10.54 (br. s, 1H), |
| | | | 7.89 (s, 1H), 7.76 (s, 1H), 7.64 (s, 1H), |
| | | | 7.20 (d, J = 2.0 Hz, 2H), 6.97 (dd, J₁ = |
| | | | 2.4 Hz, J₂ = 8.8 Hz, 1H), 6.85 (d, J = |
| | | | 8.8 Hz, 2H), 4.27 (s, 4H), 3.92 (s, 2H), |
| | | | 2.88 (s, 3H), 2.54 (s, 3H) |
| 222 | I-64 | 415.0 | (DMSO-d₆, 400 MHz) δ 12.18 (s, 1H), |
| | | | 10.50 (s, 1H), 8.38 (s, 1H), 7.81 (s, |
| | | | 1H), 7.60 (d, J = 1.2 Hz, 1H), 7.46 (s, |
| | | | 1H), 6.76 (s, 1H), 4.15 (s, 2H), 3.34 (s, |
| | | | 3H), 2.83 (s, 3H), 2.45 (s, 3H) |
| 223 | I-65 | 363.0 | (DMSO-d6, 400 MHz) δ 10.54 (s, 1H), |
| | | | 7.89 (s, 1H), 7.76 (s, 1H), 7.64 (s, 1H), |
| | | | 7.20 (d, J = 2.0 Hz, 2H), 6.97 (dd, J1 = |
| | | | 2.4 Hz, J2 = 8.8 Hz, 1H), 6.85 (d, J = |
| | | | 8.8 Hz, 2H), 4.27 (s, 4H), 3.92 (s, 2H), |
| | | | 2.88 (s, 3H), 2.54 (s, 3H) |
| 226 | I-69 | 373.3 | (DMSO-d₆, 400 MHz) 400 MHz) δ |
| | | | 12.51-12.46 (m, 1H), 7.80 (s, 1H), 7.64 |
| | | | (d, J = 1.2 Hz, 1H), 7.46 (s, 1H), 4.27 |
| | | | (s, 2H), 4.15 (s, 0.7H), 4.05 (d, J = 2.0 |
| | | | Hz, 1H), 2.98 (s, 2H), 2.83 (s, 1H), |
| | | | 2.73 (s, 3H), 2.46 (s, 3H), 1.82-1.80 |
| | | | (m, 3H) |
| 227 | I-70 | 361.1 | (CD3OD, 400 MHz) δ 7.79 (s, 1H), |
| | | | 7.68 (s, 1H), 7.29 (s, 1H), 5.69-5.64 |
| | | | (m, 1H), 5.47-5.43 (m, 1H), 3.88 (s, |
| | | | 2H), 3.74 (d, J = 6.0 Hz, 2H), 2.79 (s, |
| | | | 3H), 2.51 (s, 3H), 1.70-1.68 (m, 3H) |
| 228 | I-71 | 385.2 | (DMSO-d6, 400 MHz) δ 12.39 (s, 1H), |
| | | | 8.44 (t, J = 5.2 Hz, 1H), 7.82 (s, 1H), |
| | | | 7.61 (d, J = 1.2 Hz, 1H), 7.48 (d, J = |
| | | | 2.0 Hz, 1H), 3.88-3.78 (m, 4H), 2.71 |
| | | | (s, 3H), 2.46 (s, 3H), 1.32-1.25 (m, |
| | | | 1H), 0.78-0.70 (m, 2H), 0.60-0.50 (m, |
| | | | 2H) |
| 229 | I-72 | 387.1 | (CDCl₃, 400 MHz) δ 11.20 (br s, 1H), |
| | | | 7.74 (s, 1H), 7.67 (s, 1H), 7.18 (d, J = |
| | | | 2.0 Hz, 1H), 6.23 (s, 1H), 3.81 (s, 2H), |
| | | | 2.78 (s, 3H), 2.52 (s, 3H), 1.84 (s, 3H), |
| | | | 1.69 (s, 6H) |

The following compound was synthesized in a similar manner as 214 using I-68 and replacing I-59 with the reactant indicated.

| | | LCMS | |
|---|---|---|---|
| Example | Reactant | m/z | ¹H-NMR |
| 225 | I-6 | 434.1 | (DMSO-d6, 400 MHz) δ 12.50 (s, 1H), |
| | | | 10.15 (s, 1H), 8.14 (s, 1H), 7.73 (s, |
| | | | 1H), 7.57 (d, J = 7.6 Hz, 1H), 7.53 (s, |
| | | | 1H), 6.57 (s, 1H), 6.26 (dd, J1 = 2.4 |
| | | | Hz, J2 = 7.2 Hz, 1H), 4.18 (s, 2H), |
| | | | 3.30 (s, 3H), 2.89 (s, 3H) |

### Synthesis of 2-((2-cyano-N,5-dimethyl-1H-indole)-7-sulfonamido)-N-(6-methoxypyridin-3-yl)acetamide (Example 216)

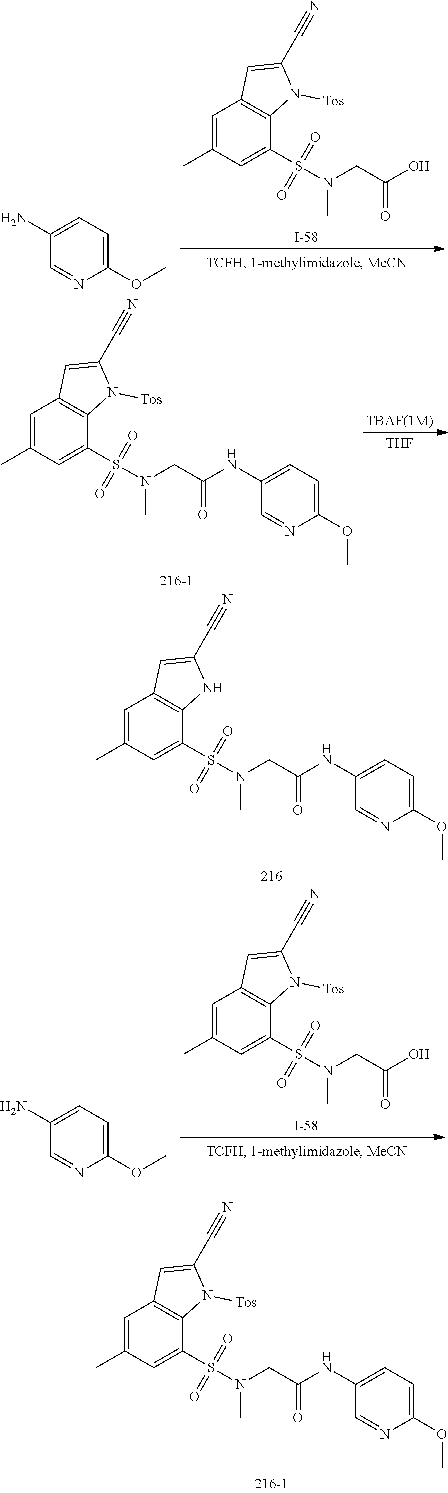

To a solution of I-58 (0.090 g, 0.195 mmol) in acetonitrile (5 mL) was added 6-methoxypyridin-3-amine (0.024 g, 0.195 mmol), 1-methylimidazole (0.080 g, 0.975 mmol) and TCFH (0.109 g, 0.390 mmol) at 0° C. The mixture was stirred at 40° C. for 4 hours then added to water (20 mL) at 25° C., and extracted with ethyl acetate (20 mL*2). The combined organic layers were washed with brine (20 mL*2), dried over anhydrous sodium sulfate, filtered, and the filtrate was concentrated under reduced pressure. The residue was purified by column chromatography (silica gel, Petroleum ether/Ethyl acetate=99/1 to 90/10) to afford 2-(2-cyano-N,5-dimethyl-1-tosyl-1H-indole-7-sulfonamido)-N-(6-methoxypyridin-3-yl)acetamide (216-1) (0.060 g, 54.2% yield) as a red oil.

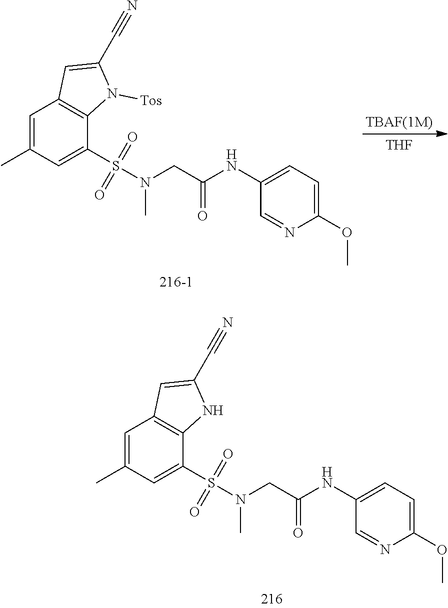

To a solution of 216-1 (0.060 g, 0.105 mmol) in tetrahydrofuran (2 mL) was added tetrabutyl-ammonium fluoride (1 mol/L in tetrahydrofuran, 0.317 mL) at 0° C. The reaction mixture was diluted with water (8 mL) and extracted with ethyl acetate (10 mL*4). The combined organic layers were washed with 0.1 mol/L aqueous solution of hydrochloric acid (20 mL*3) and brine (20 mL*2), then dried over anhydrous sodium sulfate, filtered, and the filtrate was concentrated under reduced pressure. The residue was triturated with dimethyl sulphoxide/water (v/v 0.5 mL/5 mL) and then acetonitrile (1 mL) to afford 2-(2-cyano-N, 5-dimethyl-1H-indole-7-sulfonamido)-N-(6-methoxypyridin-3-yl)acetamide (216) (0.036 g, 82.1% yield) as a pink solid.

LCMS of 216: m/z 414.2 [M+H]⁺

¹H-NMR of 216: (DMSO-d₆, 400 MHz) δ 12.31 (s, 1H), 10.09 (s, 1H), 8.28 (d, J=2.4 Hz, 1H), 7.82-7.79 (m, 2H), 7.63 (s, 1H), 7.47 (s, 1H), 6.79 (d, J=8.8 Hz, 1H), 4.08 (s, 2H), 3.81 (s, 3H), 2.84 (s, 3H), 2.44 (s, 3H)

### Synthesis of 2-((2-cyano-N,5-dimethyl-1H-indole)-7-sulfonamido)-N-(4-oxo-6,7,8,9-tetrahydro-4H-pyrido[1,2-a]pyrimidin-2-yl)acetamide (Example 224)

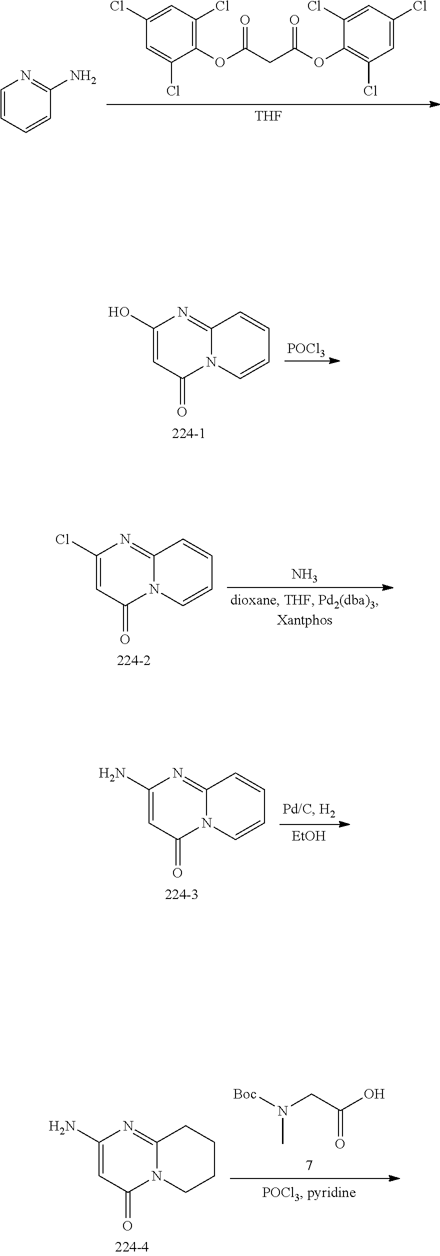

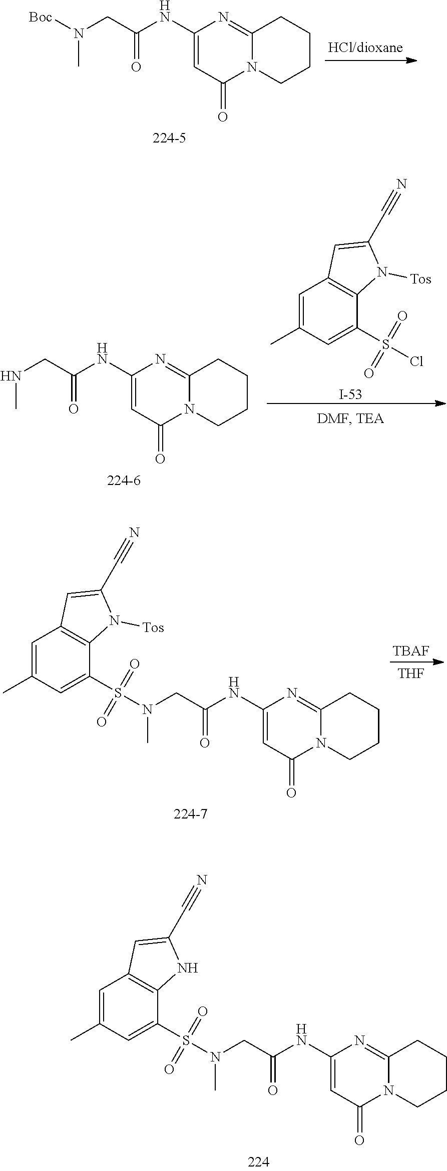

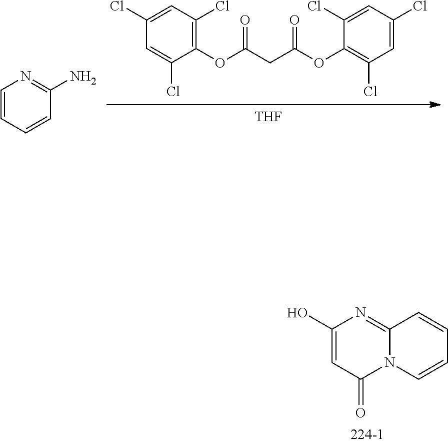 [SMILES: Nc1ccccn1 ; O=C(CC(=O)Oc1c(Cl)cc(Cl)cc1Cl)Oc1c(Cl)cc(Cl)cc1Cl ; O=c1cc(O)nc2ccccn12 ; "THF"]

To a solution of bis(2,4,6-trichlorophenyl) malonate (9.84 g, 21.3 mmol) in tetrahydrofuran (200 mL) was added pyridin-2-amine (2.00 g, 21.3 mmol) in tetrahydrofuran (100 mL). The mixture was stirred at 60° C. for 12 hours, causing a yellow suspension to precipitate out. The reaction was filtered and washed by tetrahydrofuran (50 mL) to afford 2-hydroxy-4H-pyrido[1,2-a]pyrimidin-4-one (224-1) (2.85 g, 17.6 mmol, 82.7% yield) as a yellow solid.

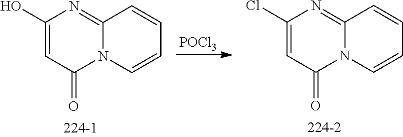

To solution of 224-1 (2.50 g, 15.4 mmol) was added phosphorus oxychloride (18 mL) at 25° C. The mixture was stirred at 80° C. for 3 hours. The reaction mixture was concentrated under reduced pressure to removed phosphorus oxychloride, then the crude residue was suspended in water (50 mL). Ethyl acetate (50 mL) was added and the aqueous phase was separated then extracted with ethyl acetate (50*2 mL). The combined organic layers were washed with brine (50 mL*2), dried over anhydrous sodium sulfate, filtered, and the filtrate was concentrated under reduced pressure. The residue was purified by reversed-phase flash (0.1% HCl in water/acetonitrile condition) to afford 2-chloro-4H-pyrido[1,2-a]pyrimidin-4-one (224-2) (1.00 g, 5.20 mmol, 36.0% yield) as a yellow solid.

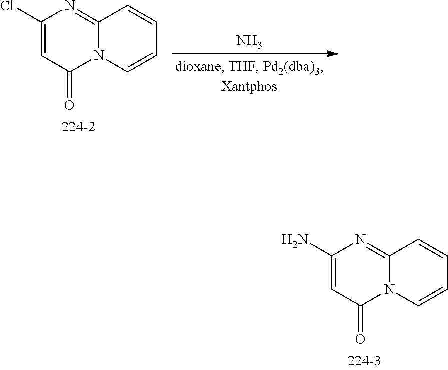

A solution of 224-2 (0.800 g, 4.43 mmol) in tetrahydrofuran (15 mL) and dioxane (15 mL) was bubbled with ammonia (15 psi) for 30 minutes at 0° C. To the mixture was added tris(dibenzylideneacetone)dipalladium(0) (0.405 g, 0.440 mmol) followed by 4,5-bis(diphenylphosphino)-9,9-dimethylxanthene (0.510 g, 0.880 mmol). The mixture was stirred at 130° C. for 12 hours under nitrogen atmosphere in sealed tube. After that the mixture was filtered and the filtrate was purified by reversed-phase flash (0.1% NH₃·H₂O in water/acetonitrile condition) to afford 2-amino-4H-pyrido[1,2-a]pyrimidin-4-one (224-3) (0.700 g, 4.34 mmol, 98.1% yield) as a yellow solid.

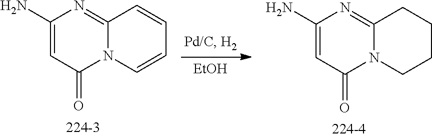 [SMILES: "EtOH" ; Nc1cc(=O)n2ccccc2n1 ; Nc1cc(=O)n2c(n1)CCCC2]

To a solution of 224-3 (0.340 g, 2.11 mmol) in methanol (20 mL) was added Pd/C (0.050 g, 2.11 mmol, 10% on activated carbon), under a nitrogen atmosphere, and the solution was stirred at 25° C. for 4 hours under hydrogen (15 psi). After that the mixture was filtered through a bed of celite and washed with methanol (10 mL). The filtrate was concentrated under reduced pressure to afford 2-amino-6,7,8,9-tetrahydro-4H-pyrido[1,2-a]pyrimidin-4-one (224-4) (0.220 g, 1.33 mmol, 63.1% yield) as a white solid.

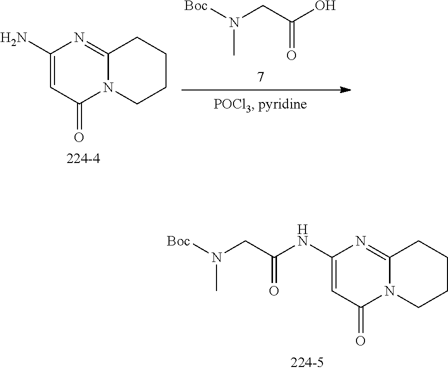 [SMILES: Nc1cc(=O)n2c(n1)CCCC2 ; CN(CC(=O)O)C(=O)OC(C)(C)C ; CN(CC(=O)Nc1cc(=O)n2c(n1)CCCC2)C(=O)OC(C)(C)C]

To a solution of 224-4 (0.100 g, 0.605 mmol) and 2-((tert-butoxycarbonyl)(methyl)amino)acetic acid (0.114 g, 0.605 mmol) in pyridine (6 mL) was added phosphorus oxychloride (0.280 g, 1.82 mmol) at 0° C., then mixture was stirred at 0° C. for 1 hour. Then the reaction was quenched with a saturated aqueous solution of ammonium bicarbonate (30 mL) and extracted with ethyl acetate (30 mL*2). The combined organic layers were washed with brine (30 mL*2), dried over anhydrous sodium sulfate, filtered, and the filtrate was concentrated under reduced pressure. The residue was purified by column chromatography (silica gel, Petroleum ether/Ethyl acetate=99/1 to 70/30) to afford tert-butyl methyl(2-oxo-2-((4-oxo-6,7,8,9-tetrahydro-4H-pyrido[1,2-a]pyrimidin-2-yl)amino)ethyl)carbamate (224-5) (0.090 g, 0.267 mmol, 44.2% yield) as a yellow solid.

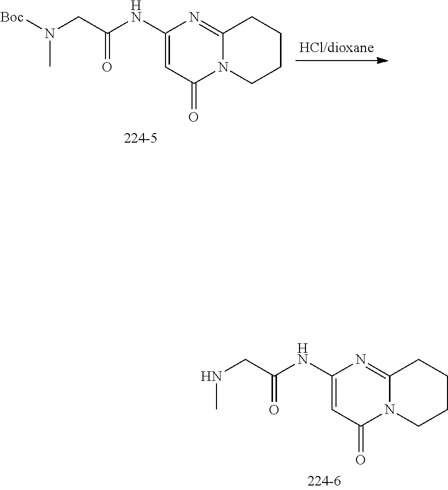 [SMILES: CN(CC(=O)Nc1cc(=O)n2c(n1)CCCC2)C(=O)OC(C)(C)C ; CNCC(=O)Nc1cc(=O)n2c(n1)CCCC2]

To a solution of 224-5 (0.085 g, 0.252 mmol) in dioxane (2 mL) was added a solution of hydrochloric acid in dioxane (4 mol/L, 2 mL), then mixture was stirred at 25° C. for 0.5 hour. After that the reaction mixture was concentrated under reduced pressure to afford 2-(methylamino)-N-(4-oxo-6,7,8,9-tetrahydro-4H-pyrido[1,2-a]pyrimidin-2-yl)acetamide as a hydrochloric salt (224-6) (0.090 g, crude) as a yellow solid.

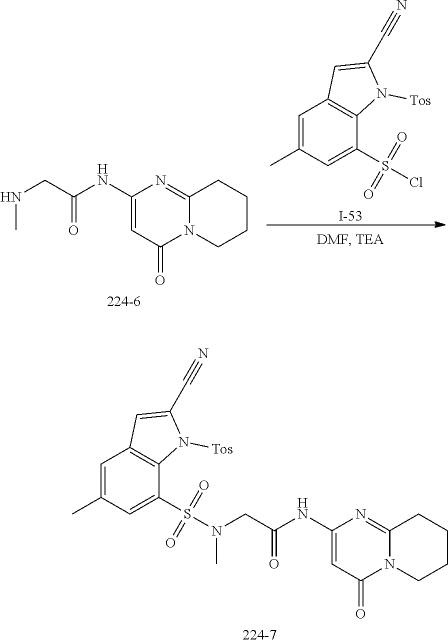

To a solution of 224-6 (0.078 g, 0.286 mmol, hydrochloric salt) and triethylamine (0.111 g, 1.10 mmol) in N,N-dimethylformamide (2 mL) was added I-53 (0.090 g, 0.220 mmol) at 0° C., then mixture was stirred at 25° C. for 0.5 hour. After that the reaction mixture was diluted with water (15 mL) and extracted with ethyl acetate (15 mL*2). The combined organic layers were washed with brine (15 mL*2), dried over anhydrous sodium sulfate, filtered, and the filtrate concentrated under reduced pressure to give a residue. The residue was purified by column chromatography (silica gel, Petroleum ether/Ethyl acetate=99/1 to 0/100) to afford 2-(2-cyano-N,5-dimethyl-1-tosyl-1H-indole-7-sulfonamido)-N-(4-oxo-6,7,8,9-tetrahydro-4H-pyrido[1,2-a]pyrimidin-2-yl)acetamide (224-7) (0.090 g, 0.148 mmol, 67.17% yield) as a white solid.

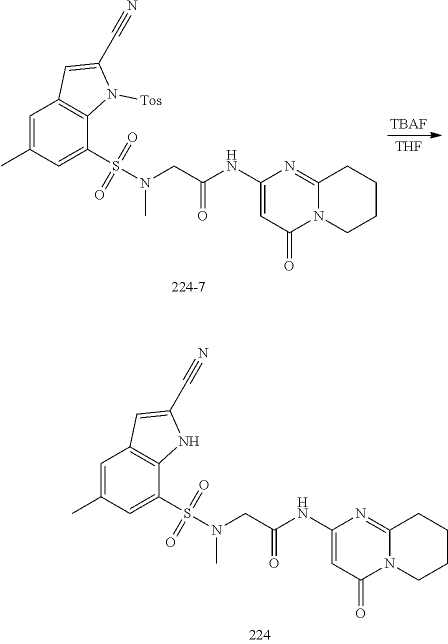

To a solution of 224-7 (0.085 g, 0.139 mmol) in tetrahydrofuran (2 mL) was added tetrabutyl-ammonium fluoride (1 M in tetrahydrofuran, 0.418 mL) at 0° C., then mixture was stirred at 25° C. for 0.5 hour. Then the reaction mixture was diluted with water (8 mL) and extracted with ethyl acetate (10 mL*4). The combined organic phases was washed with 0.1 mol/L aqueous solution of hydrochloric acid (20 mL*3) followed by brine (20 mL*2) then dried over anhydrous sodium sulfate, filtered, and the filtrate was concentrated under reduced pressure. The residue was purified by column chromatography (silica gel, Petroleum ether/Ethyl acetate=99/1 to 0/100) to give the residue. The crude product was triturated with dimethyl sulphoxide/water (v/v 0.5 mL/5 mL) for twice and acetonitrile (0.5 mL*2) to afford 2-(2-cyano-N,5-dimethyl-1H-indole-7-sulfonamido)-N-(4-oxo-6,7,8,9-tetrahydro-4H-pyrido[1,2-a]pyrimidin-2-yl)acetamide (224) (0.014 g, 0.030 mmol, 27.7% yield) as a white solid.

LCMS of 224: m/z 455.2 [M+H]⁺

¹H-NMR of 224: (CD₃CN, 400 MHz) δ 11.01 (br. s, 1H), 8.41 (br. s, 1H), 7.80 (s, 1H), 7.69 (s, 1H), 7.30 (s, 1H), 6.72 (s, 1H), 4.07 (s, 2H), 3.80 (t, J=6.4 Hz, 2H), 2.83 (s, 3H), 2.76 (t, J=6.8 Hz, 2H), 2.49 (s, 3H), 1.91-1.88 (m, 2H), 1.86-1.81 (m, 2H)

| | | | | Human |
|---|---|---|---|---|
| | | | LPD IC₅₀ | Liver |
| | | LPD | (μM) w/ | Microsome |
| | | IC₅₀ | 30 min | Stability |
| Example | Compound Structure | (μM) | preincubation | (μL/min/mg) |
| 1 | 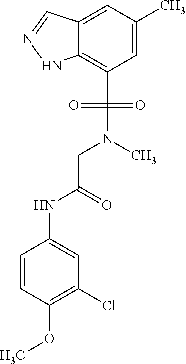 | 0.0204 | | >768 |
| 2 | 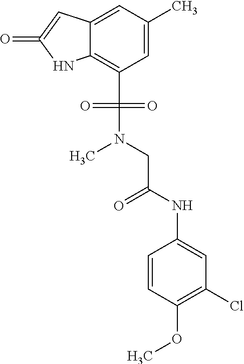 | 0.075 | | 490 |
| 3 | 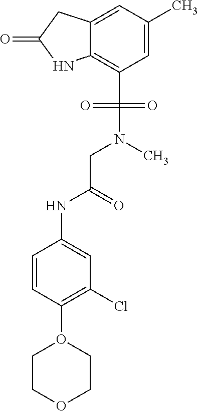 | >10 | | 5 |
| 4 | 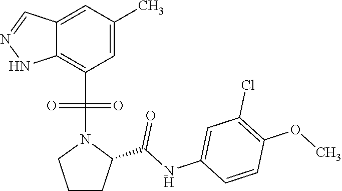 | 0.39 | | 324 |
| 5 | 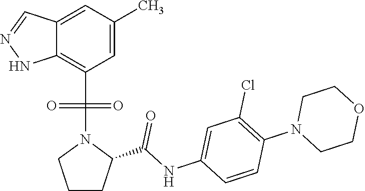 | 0.042 | | 133 |
| 6 | 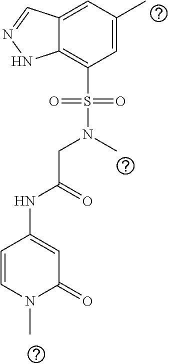 [SMILES: Cc1cc(S(=O)(=O)N(C)CC(=O)Nc2ccn(C)c(=O)c2)c2[nH]ncc2c1] | 0.0489 | 0.022 | −11 |
| 7 | 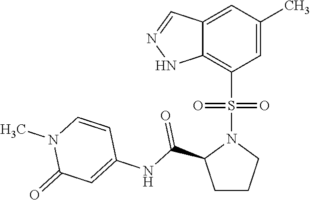 | 0.0513 | | 18 |
| 8 | 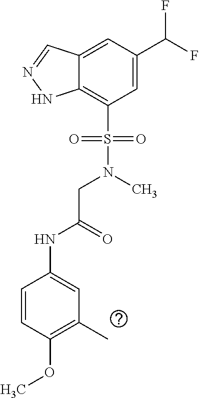 [SMILES: COc1ccc(NC(=O)CN(C)S(=O)(=O)c2cc(C(F)F)cc3cn[nH]c23)cc1C] | 0.0263 | | 534 |
| 9 | 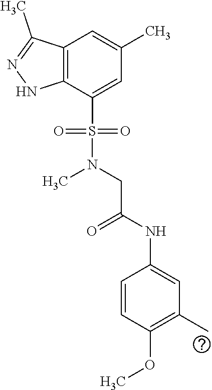 [SMILES: COc1ccc(NC(=O)CN(C)S(=O)(=O)c2cc(C)cc3c(C)n[nH]c23)cc1C] | 0.0273 | | >768 |
| 10 | 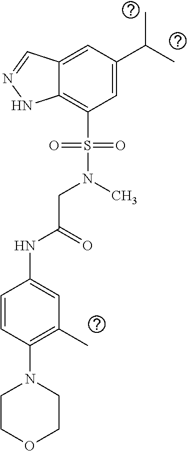 [SMILES: Cc1cc(NC(=O)CN(C)S(=O)(=O)c2cc(C(C)C)cc3cn[nH]c23)ccc1N1CCOCC1] | 0.0319 | | 198 |
| 11 | 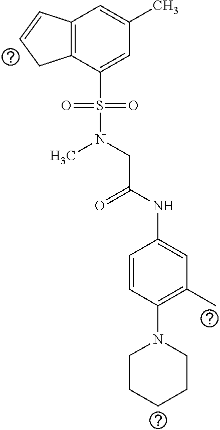 [SMILES: Cc1cc2c(c(S(=O)(=O)N(C)CC(=O)Nc3ccc(N4CCCCC4)c(C)c3)c1)CC=C2] | 0.0341 | | 105 |
| 12 | 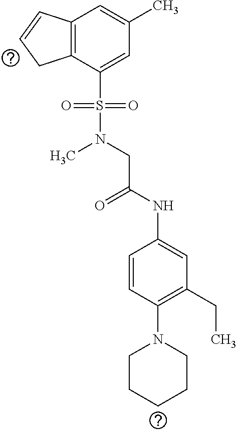 [SMILES: CCc1cc(NC(=O)CN(C)S(=O)(=O)c2cc(C)cc3c2CC=C3)ccc1N1CCCCC1] | 0.0287 | | 179 |
| 13 | 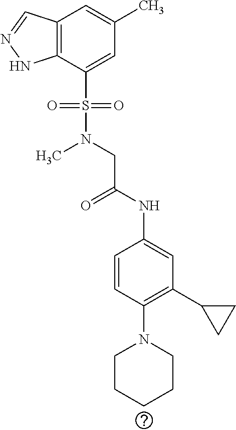 [SMILES: Cc1cc(S(=O)(=O)N(C)CC(=O)Nc2ccc(N3CCCCC3)c(C3CC3)c2)c2[nH]ncc2c1] | 0.0214 | | 164 |
| 14 | 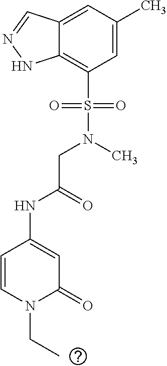 [SMILES: CCn1ccc(NC(=O)CN(C)S(=O)(=O)c2cc(C)cc3cn[nH]c23)cc1=O] | 0.0531 | | −12 |
| 15 | 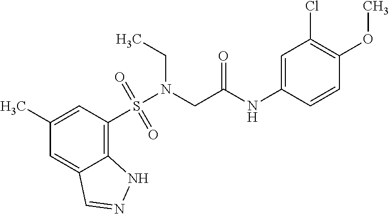 | 0.0417 | | 528 |
| 16 | 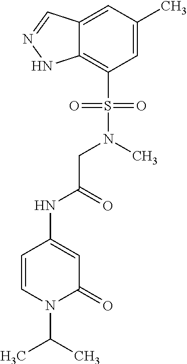 [SMILES: Cc1cc(S(=O)(=O)N(C)CC(=O)Nc2ccn(C(C)C)c(=O)c2)c2[nH]ncc2c1] | 0.0508 | | 35 |
| 17 | 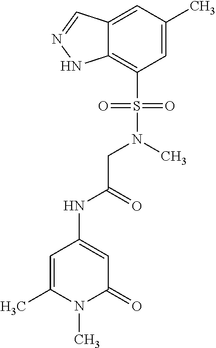 | 0.052 | | |
| 18 | 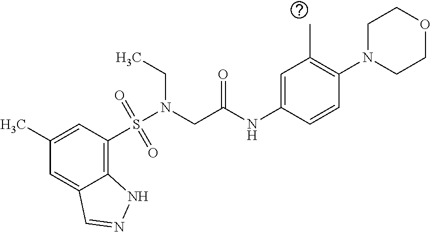 [SMILES: CCN(CC(=O)Nc1ccc(N2CCOCC2)c(C)c1)S(=O)(=O)c1cc(C)cc2cn[nH]c12] | 0.0469 | | 131 |
| 19 | 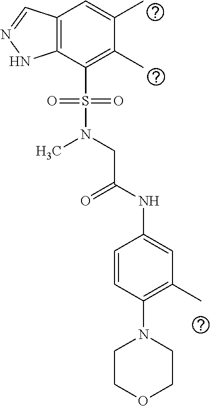 [SMILES: Cc1cc(NC(=O)CN(C)S(=O)(=O)c2c(C)c(C)cc3cn[nH]c23)ccc1N1CCOCC1] | 0.0469 | | 253 |
| 20 | 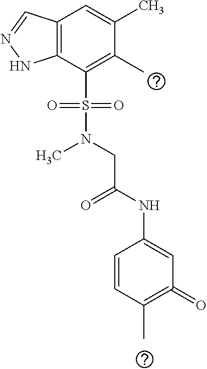 | 0.0868 | | 58 |
| 21 | 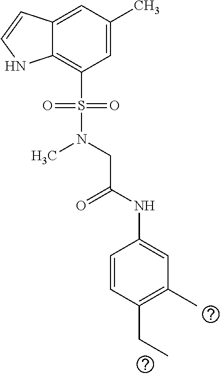 [SMILES: CCc1ccc(NC(=O)CN(C)S(=O)(=O)c2cc(C)cc3cc[nH]c23)cc1C] | 0.136 | | 328 |
| 22 | 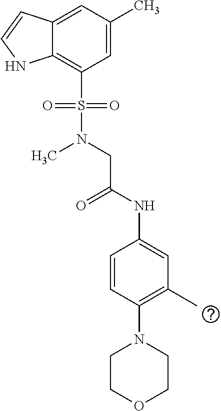 [SMILES: Cc1cc(S(=O)(=O)N(C)CC(=O)Nc2ccc(N3CCOCC3)c(C)c2)c2[nH]ccc2c1] | 0.153 | | 101 |
| 23 | 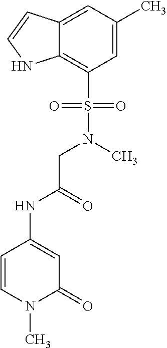 [SMILES: Cc1cc(S(=O)(=O)N(C)CC(=O)Nc2ccn(C)c(=O)c2)c2[nH]ccc2c1] | 0.0993 | | 19 |
| 24 | 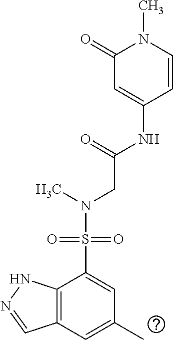 [SMILES: Cc1cc(S(=O)(=O)N(C)CC(=O)Nc2ccn(C)c(=O)c2)c2[nH]ncc2c1] | 0.0553 | | 33 |
| 25 | 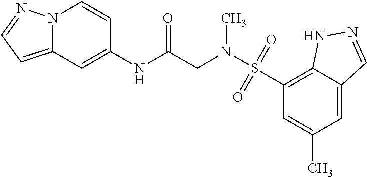 [SMILES: Cc1cc(S(=O)(=O)N(C)CC(=O)Nc2ccn3nccc3c2)c2[nH]ncc2c1] | 0.0204 | | 275 |
| 26 | 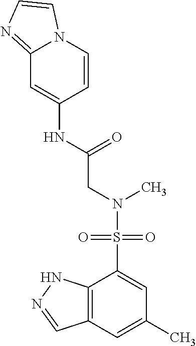 [SMILES: Cc1cc(S(=O)(=O)N(C)CC(=O)Nc2ccn3ccnc3c2)c2[nH]ncc2c1] | 0.0628 | | 154 |
| 27 | 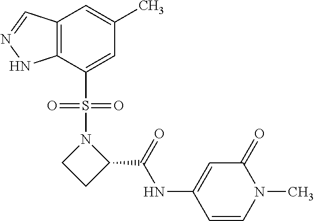 [SMILES: Cc1cc(S(=O)(=O)N2CC[C@H]2C(=O)Nc2ccn(C)c(=O)c2)c2[nH]ncc2c1] | 0.0545 | | −8 |
| 28 | 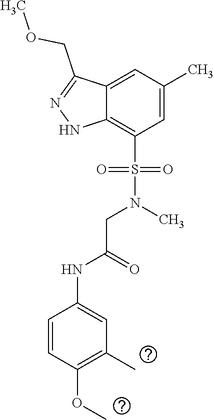 | 3640 | | >768 |
| 29 | 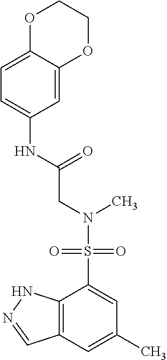 [SMILES: Cc1cc(S(=O)(=O)N(C)CC(=O)Nc2ccc3c(c2)OCCO3)c2[nH]ncc2c1] | 0.0109 | | 76 |
| 30 | 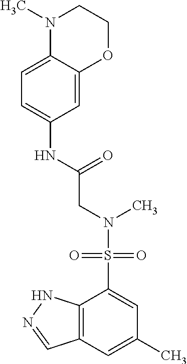 [SMILES: Cc1cc(S(=O)(=O)N(C)CC(=O)Nc2ccc3c(c2)OCCN3C)c2[nH]ncc2c1] | 0.00363 | | 78 |
| 31 | 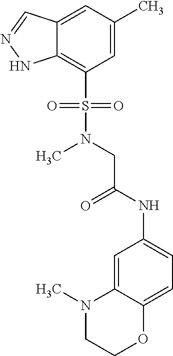 [SMILES: Cc1cc(S(=O)(=O)N(C)CC(=O)Nc2ccc3c(c2)N(C)CCO3)c2[nH]ncc2c1] | 0.0165 | | 91 |
| 32 | 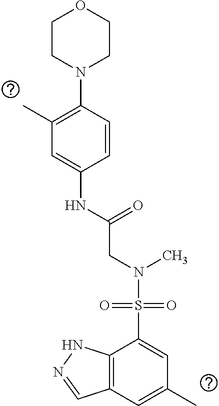 | 0.0043 | | 234 |
| 33 | 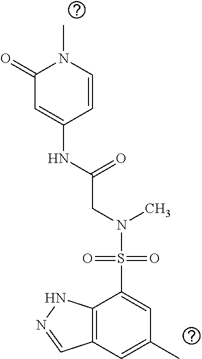 [SMILES: Cc1cc(S(=O)(=O)N(C)CC(=O)Nc2ccn(C)c(=O)c2)c2[nH]ncc2c1] | 0.0518 | | 36 |
| 34 | 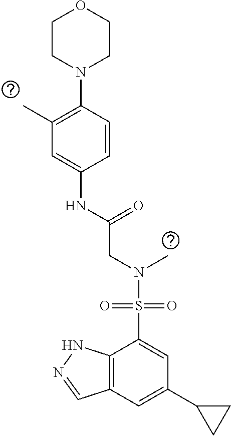 [SMILES: Cc1cc(NC(=O)CN(C)S(=O)(=O)c2cc(C3CC3)cc3cn[nH]c23)ccc1N1CCOCC1] | 0.0067 | | 203 |
| 35 | 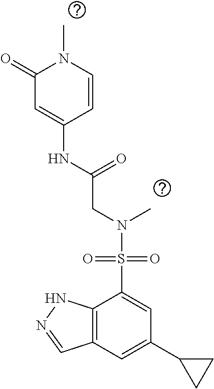 [SMILES: CN(CC(=O)Nc1ccn(C)c(=O)c1)S(=O)(=O)c1cc(C2CC2)cc2cn[nH]c12] | 0.152 | | 91 |
| 36 | 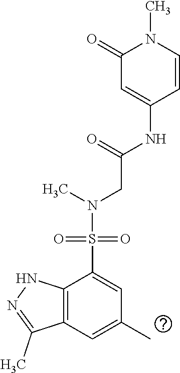 [SMILES: Cc1cc(S(=O)(=O)N(C)CC(=O)Nc2ccn(C)c(=O)c2)c2[nH]nc(C)c2c1] | 0.0657 | | −26 |
| 37 | 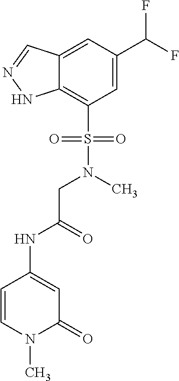 [SMILES: CN(CC(=O)Nc1ccn(C)c(=O)c1)S(=O)(=O)c1cc(C(F)F)cc2cn[nH]c12] | 0.0474 | | 22 |
| 38 | 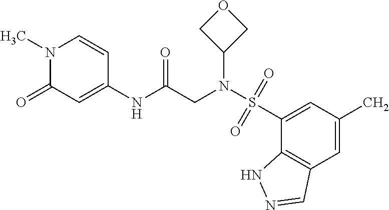 | 0.42 | | |
| 39 | 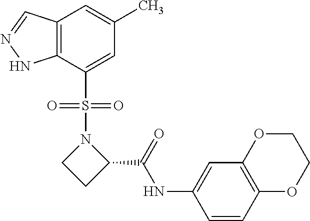 [SMILES: Cc1cc(S(=O)(=O)N2CC[C@H]2C(=O)Nc2ccc3c(c2)OCCO3)c2[nH]ncc2c1] | 0.0237 | | 76 |
| 40 | 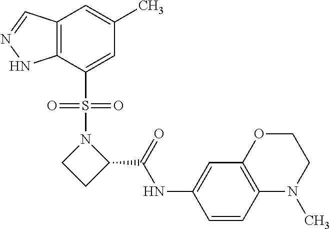 [SMILES: Cc1cc(S(=O)(=O)N2CC[C@H]2C(=O)Nc2ccc3c(c2)OCCN3C)c2[nH]ncc2c1] | 0.03 | | 78 |
| 41 | 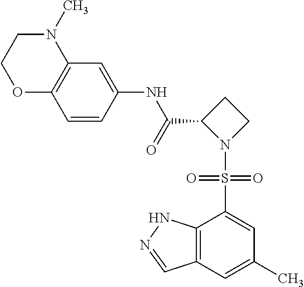 [SMILES: Cc1cc(S(=O)(=O)N2CC[C@H]2C(=O)Nc2ccc3c(c2)N(C)CCO3)c2[nH]ncc2c1] | 0.0356 | | 197 |
| 42 | 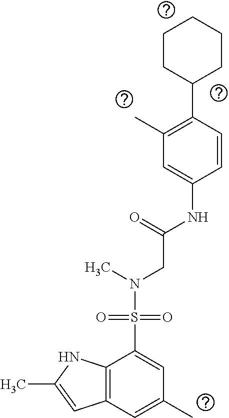 [SMILES: Cc1cc(S(=O)(=O)N(C)CC(=O)Nc2ccc(C3CCCCC3)c(C)c2)c2[nH]c(C)cc2c1] | 0.573 | | 23 |
| 43 | 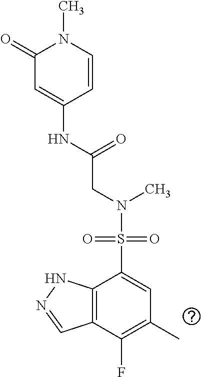 [SMILES: Cc1cc(S(=O)(=O)N(C)CC(=O)Nc2ccn(C)c(=O)c2)c2[nH]ncc2c1F] | 0.0498 | | 42 |
| 44 | 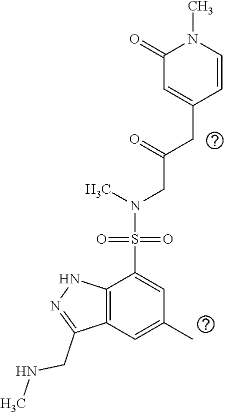 [SMILES: CNCc1n[nH]c2c(S(=O)(=O)N(C)CC(=O)Cc3ccn(C)c(=O)c3)cc(C)cc12] | 0.13 | | −2 |
| 45 | 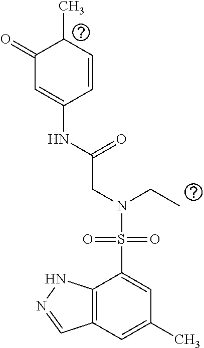 [SMILES: CCN(CC(=O)NC1=CC(=O)C(C)C=C1)S(=O)(=O)c1cc(C)cc2cn[nH]c12] | 0.0268 | | 39 |
| 46 | 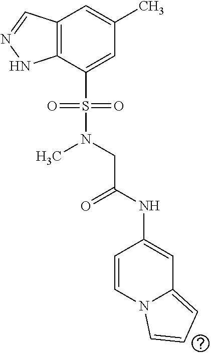 [SMILES: Cc1cc(S(=O)(=O)N(C)CC(=O)Nc2ccn3cccc3c2)c2[nH]ncc2c1] | 0.0067 | | 4 |
| 47 | 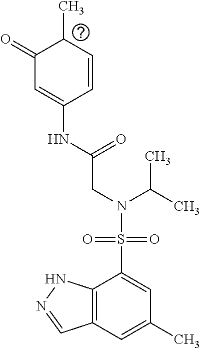 [SMILES: Cc1cc(S(=O)(=O)N(CC(=O)NC2=CC(=O)C(C)C=C2)C(C)C)c2[nH]ncc2c1] | 0.434 | | −9 |
| 48 | 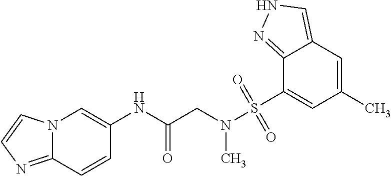 [SMILES: Cc1cc(S(=O)(=O)N(C)CC(=O)Nc2ccc3nccn3c2)c2n[nH]cc2c1] | 0.0421 | | 36 |
| 49 | 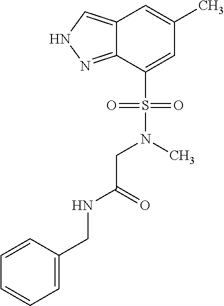 [SMILES: Cc1cc(S(=O)(=O)N(C)CC(=O)NCc2ccccc2)c2n[nH]cc2c1] | 0.031 | | 43 |
| 50 | 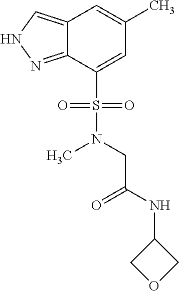 [SMILES: Cc1cc(S(=O)(=O)N(C)CC(=O)NC2COC2)c2n[nH]cc2c1] | 0.0738 | | 8 |
| 51 | 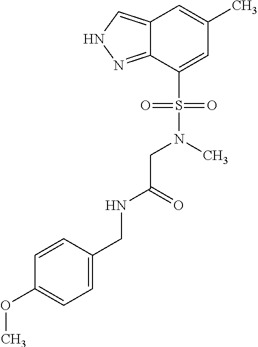 [SMILES: COc1ccc(CNC(=O)CN(C)S(=O)(=O)c2cc(C)cc3c[nH]nc23)cc1] | 0.049 | | 70 |
| 52 | 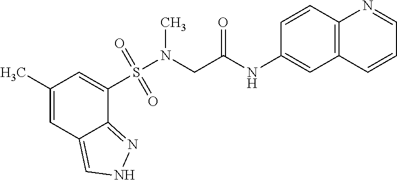 [SMILES: Cc1cc(S(=O)(=O)N(C)CC(=O)Nc2ccc3ncccc3c2)c2n[nH]cc2c1] | 0.0218 | | 113 |
| 53 | 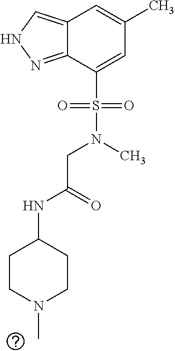 [SMILES: Cc1cc(S(=O)(=O)N(C)CC(=O)NC2CCN(C)CC2)c2n[nH]cc2c1] | 0.963 | | |
| 54 | 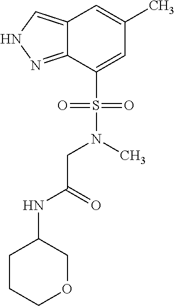 [SMILES: Cc1cc(S(=O)(=O)N(C)CC(=O)NC2CCCOC2)c2n[nH]cc2c1] | 0.0906 | | |
| 55 | 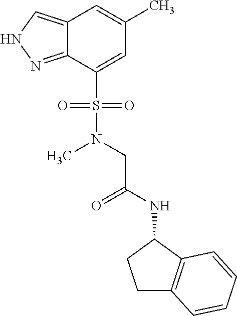 [SMILES: Cc1cc(S(=O)(=O)N(C)CC(=O)N[C@H]2CCc3ccccc32)c2n[nH]cc2c1] | 0.874 | | |
| 56 | 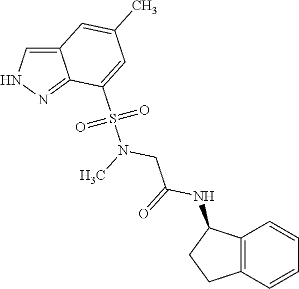 [SMILES: Cc1cc(S(=O)(=O)N(C)CC(=O)N[C@@H]2CCc3ccccc32)c2n[nH]cc2c1] | 0.0426 | | |
| 57 | 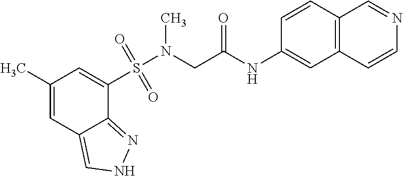 [SMILES: Cc1cc(S(=O)(=O)N(C)CC(=O)Nc2ccc3cnccc3c2)c2n[nH]cc2c1] | 0.0328 | | 59 |
| 58 | 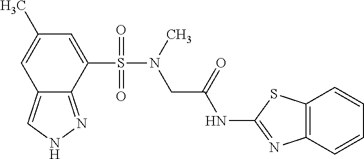 [SMILES: Cc1cc(S(=O)(=O)N(C)CC(=O)Nc2nc3ccccc3s2)c2n[nH]cc2c1] | 0.0315 | | |
| 59 | 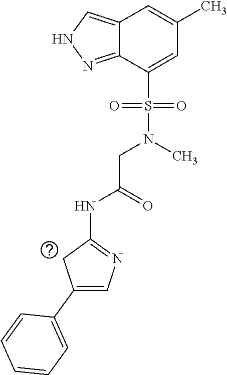 [SMILES: Cc1cc(S(=O)(=O)N(C)CC(=O)NC2=NC=C(c3ccccc3)C2)c2n[nH]cc2c1] | 0.0391 | | |
| 60 | 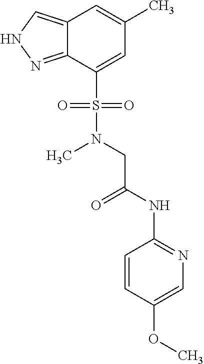 [SMILES: COc1ccc(NC(=O)CN(C)S(=O)(=O)c2cc(C)cc3c[nH]nc23)nc1] | 0.0415 | | |
| 61 | 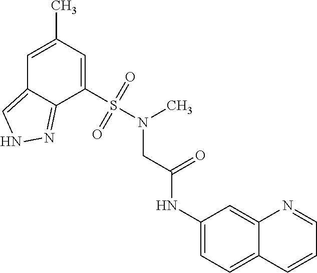 [SMILES: Cc1cc(S(=O)(=O)N(C)CC(=O)Nc2ccc3cccnc3c2)c2n[nH]cc2c1] | 0.0229 | | 122 |
| 62 | 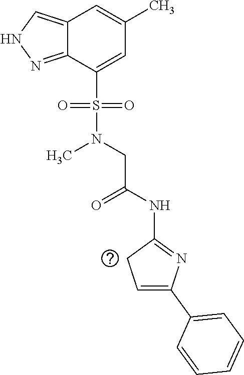 [SMILES: Cc1cc(S(=O)(=O)N(C)CC(=O)NC2=NC(c3ccccc3)=CC2)c2n[nH]cc2c1] | 0.0706 | | |
| 63 | 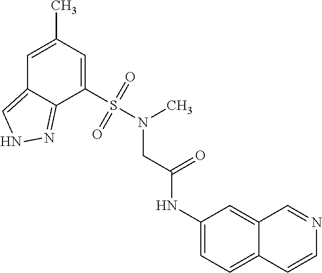 [SMILES: Cc1cc(S(=O)(=O)N(C)CC(=O)Nc2ccc3ccncc3c2)c2n[nH]cc2c1] | 0.0321 | | 63 |
| 64 | 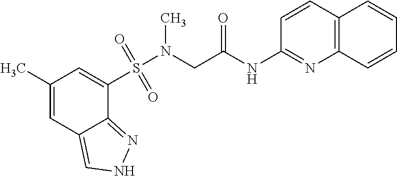 [SMILES: Cc1cc(S(=O)(=O)N(C)CC(=O)Nc2ccc3ccccc3n2)c2n[nH]cc2c1] | 0.0335 | | |
| 65 | 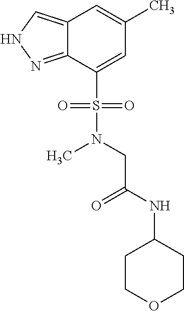 [SMILES: Cc1cc(S(=O)(=O)N(C)CC(=O)NC2CCOCC2)c2n[nH]cc2c1] | 0.118 | | |
| 66 | 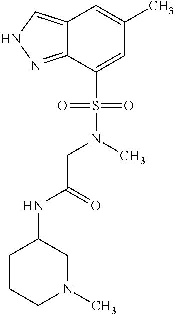 [SMILES: Cc1cc(S(=O)(=O)N(C)CC(=O)NC2CCCN(C)C2)c2n[nH]cc2c1] | 0.593 | | |
| 67 | 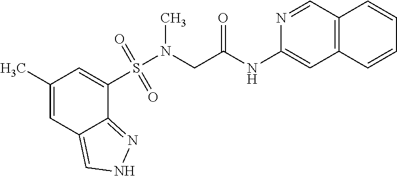 [SMILES: Cc1cc(S(=O)(=O)N(C)CC(=O)Nc2cc3ccccc3cn2)c2n[nH]cc2c1] | 0.0372 | | |
| 68 | 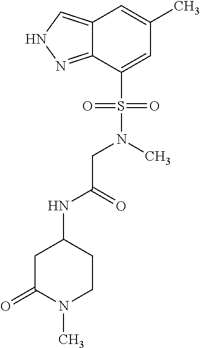 [SMILES: Cc1cc(S(=O)(=O)N(C)CC(=O)NC2CCN(C)C(=O)C2)c2n[nH]cc2c1] | 0.149 | | |
| 69 | 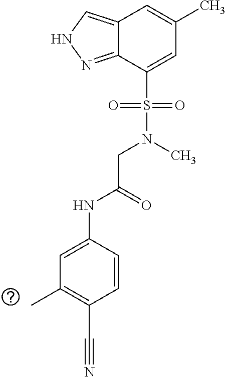 | 0.0293 | | |
| 70 | 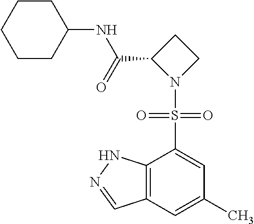 [SMILES: Cc1cc(S(=O)(=O)N2CC[C@H]2C(=O)NC2CCCCC2)c2[nH]ncc2c1] | 0.279 | | |
| 71 | 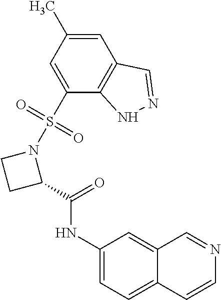 [SMILES: Cc1cc(S(=O)(=O)N2CC[C@H]2C(=O)Nc2ccc3ccncc3c2)c2[nH]ncc2c1] | 0.0513 | | 132 |
| 72 | 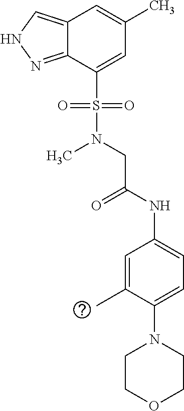 [SMILES: Cc1cc(S(=O)(=O)N(C)CC(=O)Nc2ccc(N3CCOCC3)c(C)c2)c2n[nH]cc2c1] | 0.031 | | 46 |
| 73 | 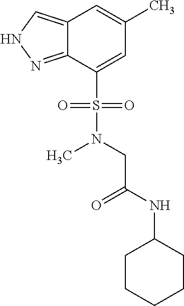 [SMILES: Cc1cc(S(=O)(=O)N(C)CC(=O)NC2CCCCC2)c2n[nH]cc2c1] | 0.0858 | | |
| 74 | 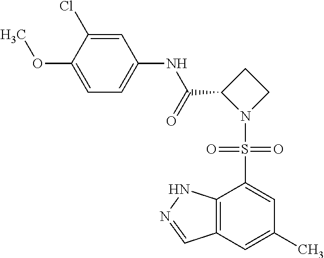 | 0.0488 | | 325 |
| 75 | 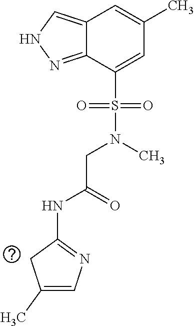 | 0.0273 | | |
| 76 | 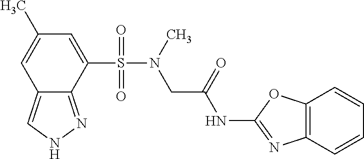 [SMILES: Cc1cc(S(=O)(=O)N(C)CC(=O)Nc2nc3ccccc3o2)c2n[nH]cc2c1] | 0.0994 | | 274 |
| 77 | 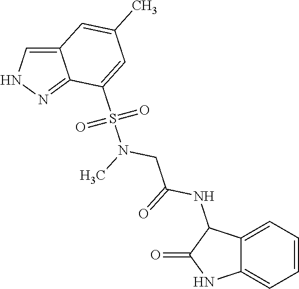 [SMILES: Cc1cc(S(=O)(=O)N(C)CC(=O)NC2C(=O)Nc3ccccc32)c2n[nH]cc2c1] | 0.803 | | |
| 78 | 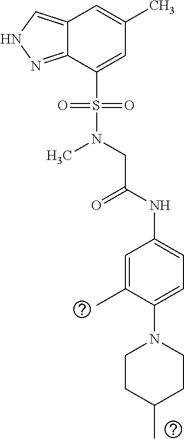 [SMILES: Cc1cc(S(=O)(=O)N(C)CC(=O)Nc2ccc(N3CCC(C)CC3)c(C)c2)c2n[nH]cc2c1] | 0.0443 | | 46 |
| 79 | 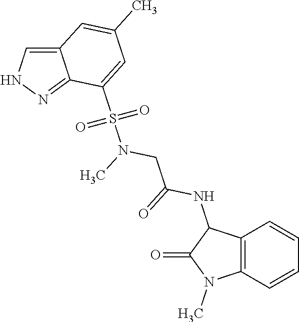 [SMILES: Cc1cc(S(=O)(=O)N(C)CC(=O)NC2C(=O)N(C)c3ccccc32)c2n[nH]cc2c1] | 0.779 | | |
| 80 | 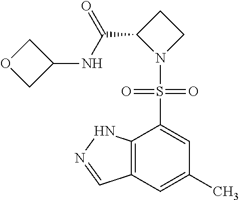 [SMILES: Cc1cc(S(=O)(=O)N2CC[C@H]2C(=O)NC2COC2)c2[nH]ncc2c1] | 0.285 | | |
| 81 | 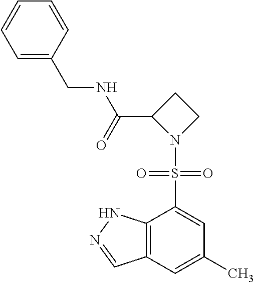 [SMILES: Cc1cc(S(=O)(=O)N2CCC2C(=O)NCc2ccccc2)c2[nH]ncc2c1] | 0.62 | | |
| 82 | 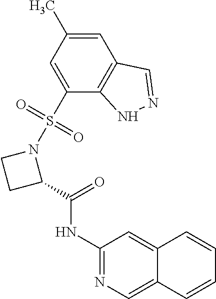 [SMILES: Cc1cc(S(=O)(=O)N2CC[C@H]2C(=O)Nc2cc3ccccc3cn2)c2[nH]ncc2c1] | 0.0489 | | |
| 83 | 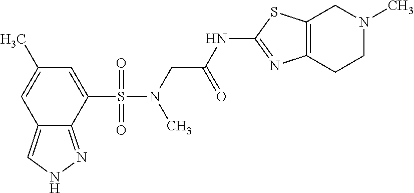 [SMILES: Cc1cc(S(=O)(=O)N(C)CC(=O)Nc2nc3c(s2)CN(C)CC3)c2n[nH]cc2c1] | 0.0978 | | 38 |
| 84 | 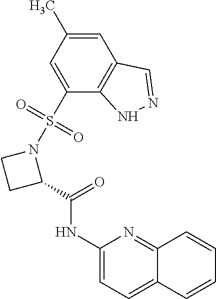 [SMILES: Cc1cc(S(=O)(=O)N2CC[C@H]2C(=O)Nc2ccc3ccccc3n2)c2[nH]ncc2c1] | 0.027 | | |
| 85 | 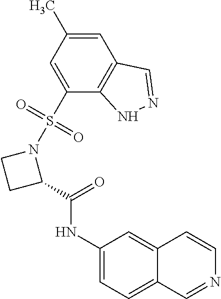 [SMILES: Cc1cc(S(=O)(=O)N2CC[C@H]2C(=O)Nc2ccc3cnccc3c2)c2[nH]ncc2c1] | 0.023 | | 93 |
| 86 | 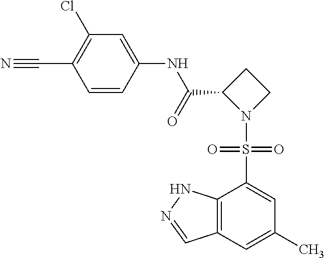 [SMILES: Cc1cc(S(=O)(=O)N2CC[C@H]2C(=O)Nc2ccc(C#N)c(Cl)c2)c2[nH]ncc2c1] | 0.0414 | | |
| 87 | 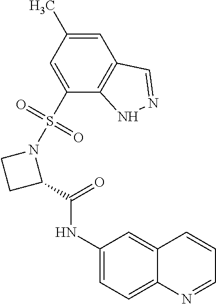 [SMILES: Cc1cc(S(=O)(=O)N2CC[C@H]2C(=O)Nc2ccc3ncccc3c2)c2[nH]ncc2c1] | 0.035 | | 111 |
| 88 | 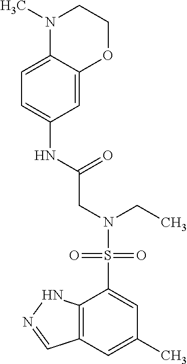 [SMILES: CCN(CC(=O)Nc1ccc2c(c1)OCCN2C)S(=O)(=O)c1cc(C)cc2cn[nH]c12] | | | 46 |
| 89 | 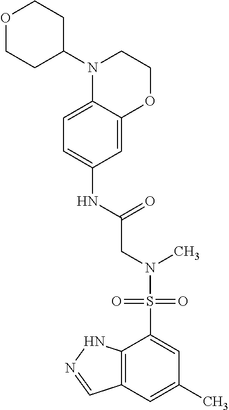 [SMILES: Cc1cc(S(=O)(=O)N(C)CC(=O)Nc2ccc3c(c2)OCCN3C2CCOCC2)c2[nH]ncc2c1] | 0.0419 | | |
| 90 | 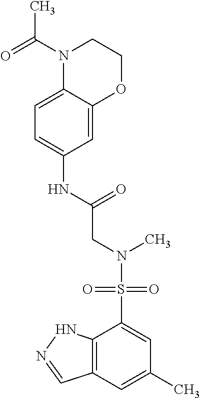 [SMILES: CC(=O)N1CCOc2cc(NC(=O)CN(C)S(=O)(=O)c3cc(C)cc4cn[nH]c34)ccc21] | | | 18 |
| 91 | 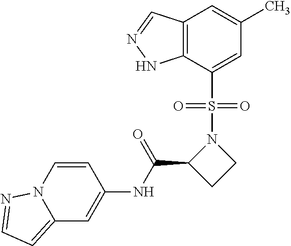 [SMILES: Cc1cc(S(=O)(=O)N2CC[C@H]2C(=O)Nc2ccn3nccc3c2)c2[nH]ncc2c1] | 0.041 | | |
| 92 | 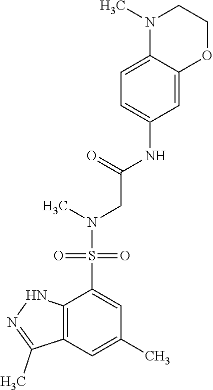 [SMILES: Cc1cc(S(=O)(=O)N(C)CC(=O)Nc2ccc3c(c2)OCCN3C)c2[nH]nc(C)c2c1] | | | 19 |
| 93 | 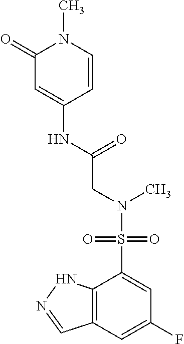 [SMILES: CN(CC(=O)Nc1ccn(C)c(=O)c1)S(=O)(=O)c1cc(F)cc2cn[nH]c12] | 0.389 | | 6 |
| 94 | 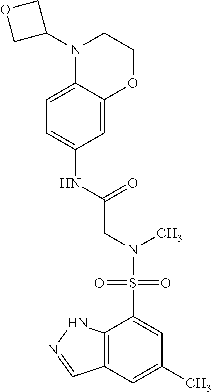 [SMILES: Cc1cc(S(=O)(=O)N(C)CC(=O)Nc2ccc3c(c2)OCCN3C2COC2)c2[nH]ncc2c1] | 0.0275 | | 45 |
| 95 | 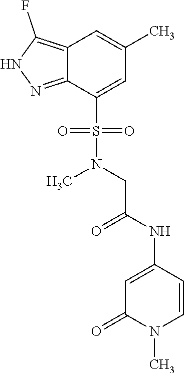 [SMILES: Cc1cc(S(=O)(=O)N(C)CC(=O)Nc2ccn(C)c(=O)c2)c2n[nH]c(F)c2c1] | 0.0513 | | 21 |
| 96 | 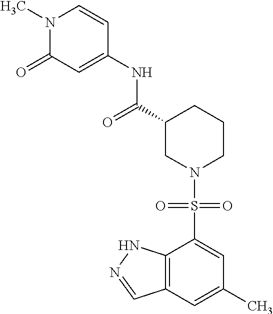 [SMILES: Cc1cc(S(=O)(=O)N2CCC[C@@H](C(=O)Nc3ccn(C)c(=O)c3)C2)c2[nH]ncc2c1] | 0.112 | | |
| 97 | 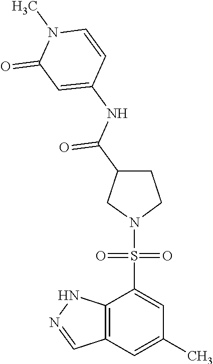 [SMILES: Cc1cc(S(=O)(=O)N2CCC(C(=O)Nc3ccn(C)c(=O)c3)C2)c2[nH]ncc2c1] | 0.0419 | | -5 |
| 98 | 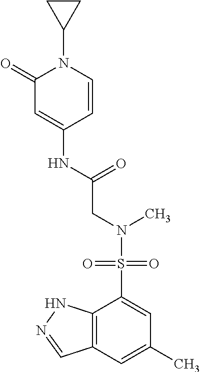 [SMILES: Cc1cc(S(=O)(=O)N(C)CC(=O)Nc2ccn(C3CC3)c(=O)c2)c2[nH]ncc2c1] | 0.093 | | 28 |
| 99 | 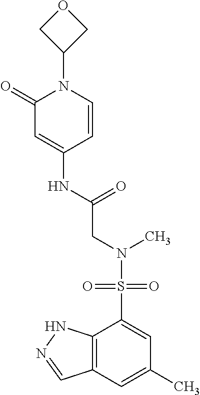 [SMILES: Cc1cc(S(=O)(=O)N(C)CC(=O)Nc2ccn(C3COC3)c(=O)c2)c2[nH]ncc2c1] | 0.056 | | 17 |
| 100 | 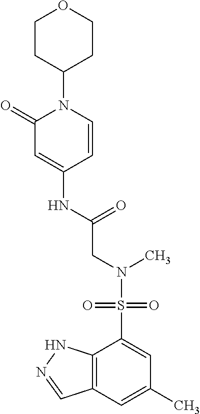 [SMILES: Cc1cc(S(=O)(=O)N(C)CC(=O)Nc2ccn(C3CCOCC3)c(=O)c2)c2[nH]ncc2c1] | 0.05 | | 6 |
| 101 | 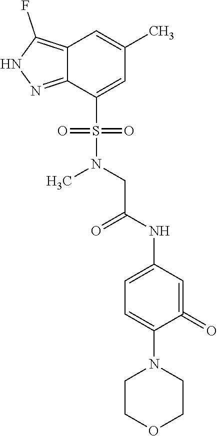 | 0.015 | | 144 |
| 102 | 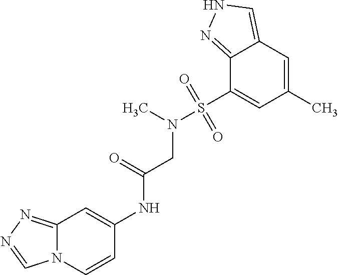 [SMILES: Cc1cc(S(=O)(=O)N(C)CC(=O)Nc2ccn3cnnc3c2)c2n[nH]cc2c1] | 0.042 | | 31 |
| 103 | 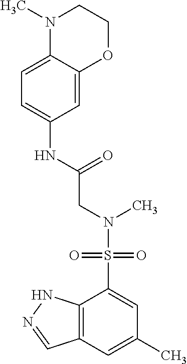 [SMILES: Cc1cc(S(=O)(=O)N(C)CC(=O)Nc2ccc3c(c2)OCCN3C)c2[nH]ncc2c1] | 0.0525 | | 146 |
| 104 | 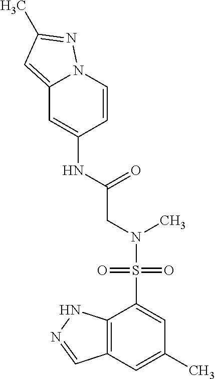 | 0.157 | | |
| 105 | 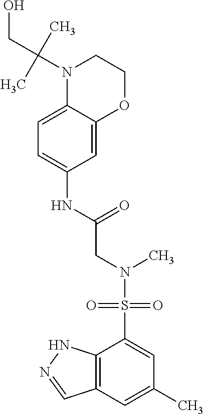 [SMILES: Cc1cc(S(=O)(=O)N(C)CC(=O)Nc2ccc3c(c2)OCCN3C(C)(C)CO)c2[nH]ncc2c1] | 0.027 | | 56 |
| 106 | 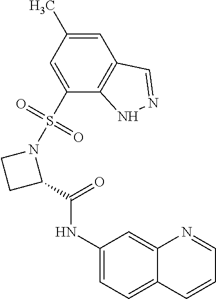 [SMILES: Cc1cc(S(=O)(=O)N2CC[C@H]2C(=O)Nc2ccc3cccnc3c2)c2[nH]ncc2c1] | 0.019 | | 236 |
| 107 | 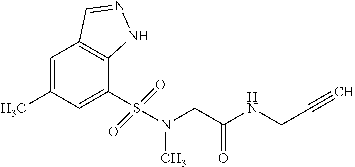 [SMILES: C#CCNC(=O)CN(C)S(=O)(=O)c1cc(C)cc2cn[nH]c12] | 0.047 | | |
| 108 | 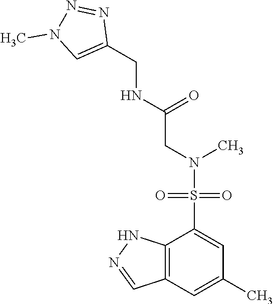 | 0.111 | | |
| 109 | 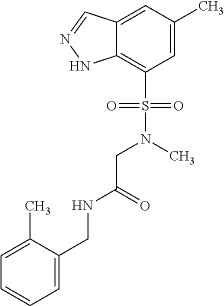 [SMILES: Cc1cc(S(=O)(=O)N(C)CC(=O)NCc2ccccc2C)c2[nH]ncc2c1] | 0.107 | | |
| 110 | 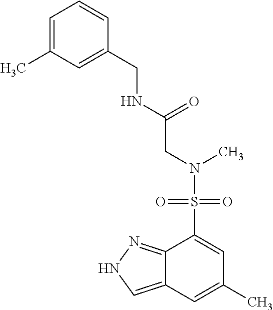 | 0.041 | | |
| 111 | 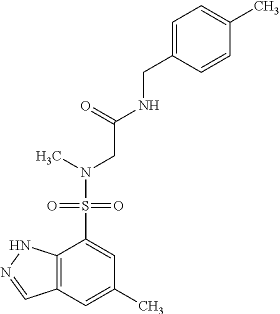 [SMILES: Cc1ccc(CNC(=O)CN(C)S(=O)(=O)c2cc(C)cc3cn[nH]c23)cc1] | 0.0522 | | |
| 112 | 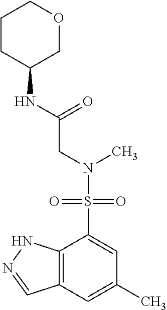 [SMILES: Cc1cc(S(=O)(=O)N(C)CC(=O)N[C@H]2CCCOC2)c2[nH]ncc2c1] | 0.109 | | |
| 113 | 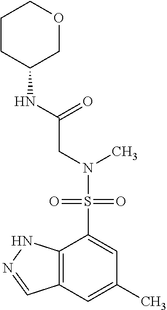 [SMILES: Cc1cc(S(=O)(=O)N(C)CC(=O)N[C@@H]2CCCOC2)c2[nH]ncc2c1] | 0.184 | | |
| 114 | 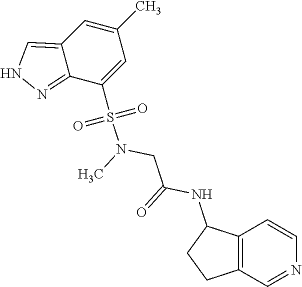 [SMILES: Cc1cc(S(=O)(=O)N(C)CC(=O)NC2CCc3cnccc32)c2n[nH]cc2c1] | 0.0954 | | |
| 115 | 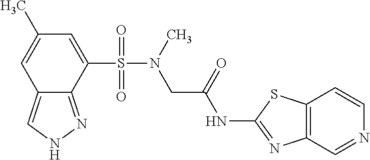 [SMILES: Cc1cc(S(=O)(=O)N(C)CC(=O)Nc2nc3cnccc3s2)c2n[nH]cc2c1] | 0.0276 | | |
| 116 | 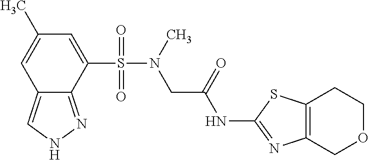 [SMILES: Cc1cc(S(=O)(=O)N(C)CC(=O)Nc2nc3c(s2)CCOC3)c2n[nH]cc2c1] | 0.0771 | | |
| 117 | 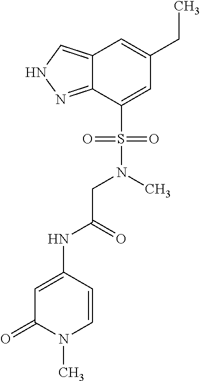 [SMILES: CCc1cc(S(=O)(=O)N(C)CC(=O)Nc2ccn(C)c(=O)c2)c2n[nH]cc2c1] | 0.05 | | |
| 118 | 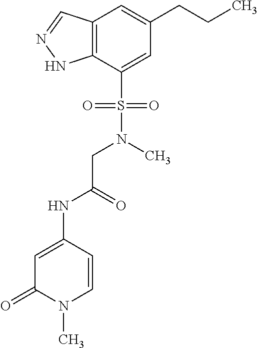 [SMILES: CCCc1cc(S(=O)(=O)N(C)CC(=O)Nc2ccn(C)c(=O)c2)c2[nH]ncc2c1] | 0.0702 | | |
| 119 | 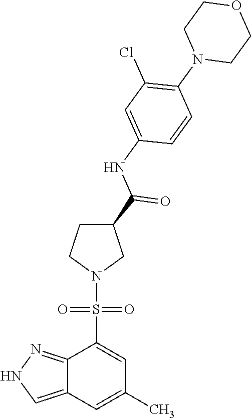 [SMILES: Cc1cc(S(=O)(=O)N2CC[C@@H](C(=O)Nc3ccc(N4CCOCC4)c(Cl)c3)C2)c2n[nH]cc2c1] | 0.0348 | | |
| 120 | 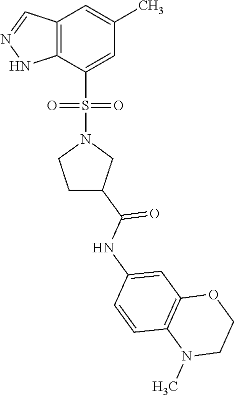 [SMILES: Cc1cc(S(=O)(=O)N2CCC(C(=O)Nc3ccc4c(c3)OCCN4C)C2)c2[nH]ncc2c1] | 0.0197 | | |
| 121 | 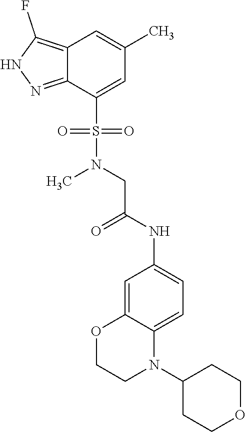 [SMILES: Cc1cc(S(=O)(=O)N(C)CC(=O)Nc2ccc3c(c2)OCCN3C2CCOCC2)c2n[nH]c(F)c2c1] | 0.0253 | | 125 |
| 122 | 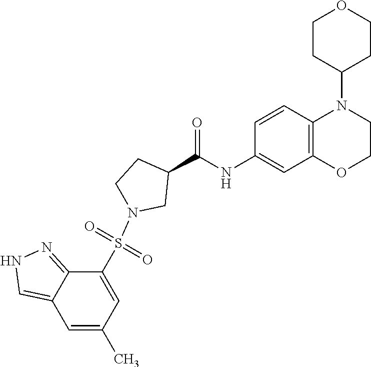 [SMILES: Cc1cc(S(=O)(=O)N2CC[C@@H](C(=O)Nc3ccc4c(c3)OCCN4C3CCOCC3)C2)c2n[nH]cc2c1] | 0.0304 | | 26 |
| 123 | 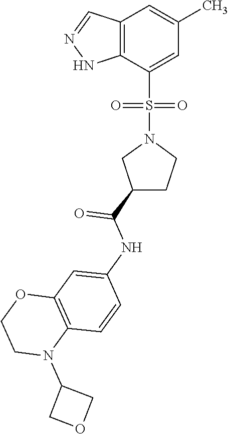 [SMILES: Cc1cc(S(=O)(=O)N2CC[C@@H](C(=O)Nc3ccc4c(c3)OCCN4C3COC3)C2)c2[nH]ncc2c1] | 0.0286 | | 23 |
| 124 | 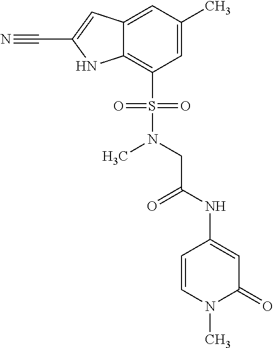 [SMILES: Cc1cc(S(=O)(=O)N(C)CC(=O)Nc2ccn(C)c(=O)c2)c2[nH]c(C#N)cc2c1] | 0.0796 | | 51 |
| 125 | 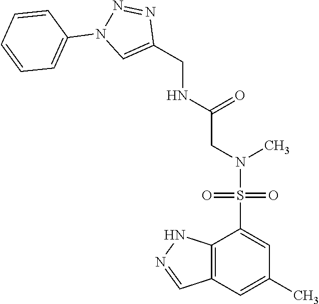 [SMILES: Cc1cc(S(=O)(=O)N(C)CC(=O)NCc2cn(-c3ccccc3)nn2)c2[nH]ncc2c1] | 0.048 | | 520 |
| 126 | 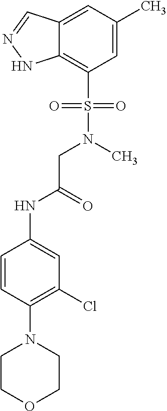 [SMILES: Cc1cc(S(=O)(=O)N(C)CC(=O)Nc2ccc(N3CCOCC3)c(Cl)c2)c2[nH]ncc2c1] | 0.019 | 0.015 | 195 |
| 127 | 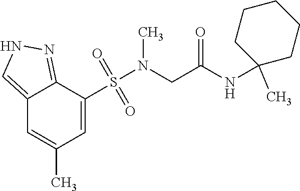 [SMILES: Cc1cc(S(=O)(=O)N(C)CC(=O)NC2(C)CCCCC2)c2n[nH]cc2c1] | 1.14 | | |
| 128 | 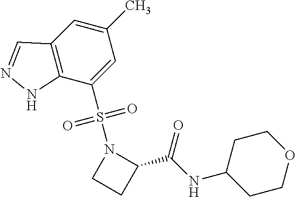 [SMILES: Cc1cc(S(=O)(=O)N2CC[C@H]2C(=O)NC2CCOCC2)c2[nH]ncc2c1] | 1.07 | | |
| 129 | 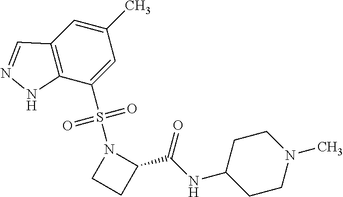 [SMILES: Cc1cc(S(=O)(=O)N2CC[C@H]2C(=O)NC2CCN(C)CC2)c2[nH]ncc2c1] | 6.56 | | 418 |
| 130 | 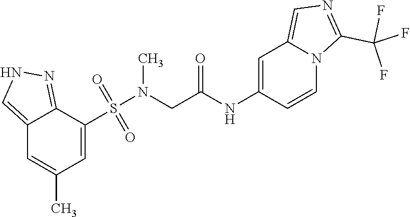 [SMILES: Cc1cc(S(=O)(=O)N(C)CC(=O)Nc2ccn3c(C(F)(F)F)ncc3c2)c2n[nH]cc2c1] | 0.0345 | | |
| 131 | 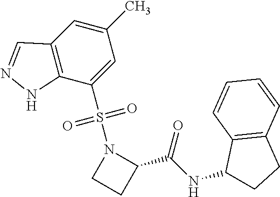 | 2.52 | | |
| 132 | 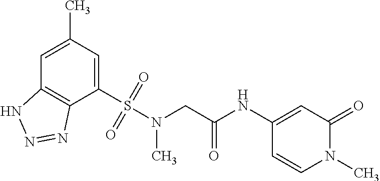 [SMILES: Cc1cc(S(=O)(=O)N(C)CC(=O)Nc2ccn(C)c(=O)c2)c2nn[nH]c2c1] | 0.386 | | |
| 133 | 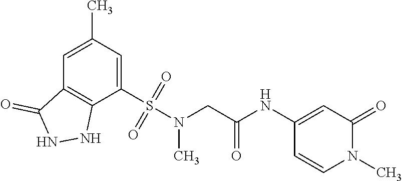 [SMILES: Cc1cc(S(=O)(=O)N(C)CC(=O)Nc2ccn(C)c(=O)c2)c2[nH][nH]c(=O)c2c1] | 0.0706 | | |
| 134 | 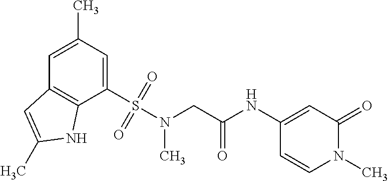 | 0.963 | | |
| 135-R | 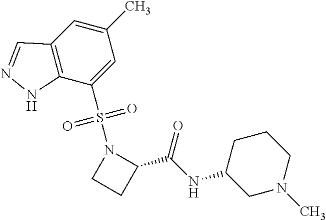 | 2.65 | | |
| 135-S | 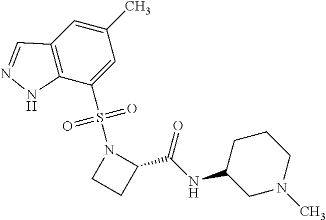 | 5.87 | | |
| 136 | 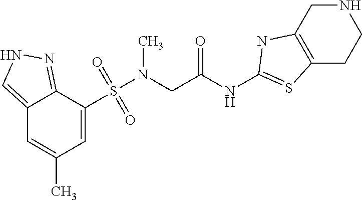 | 0.066 | | |
| 137 | 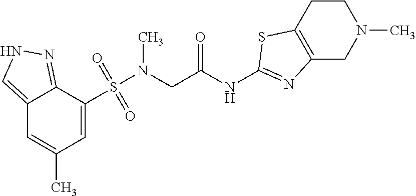 [SMILES: Cc1cc(S(=O)(=O)N(C)CC(=O)Nc2nc3c(s2)CCN(C)C3)c2n[nH]cc2c1] | 0.025 | | |
| 138 | 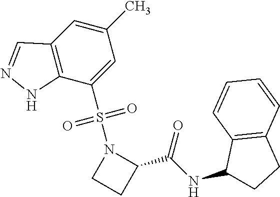 | >10 | | |
| 139 | 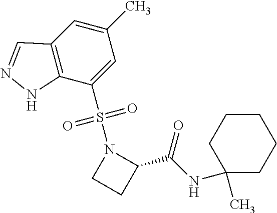 [SMILES: Cc1cc(S(=O)(=O)N2CC[C@H]2C(=O)NC2(C)CCCCC2)c2[nH]ncc2c1] | | | |
| 140 | 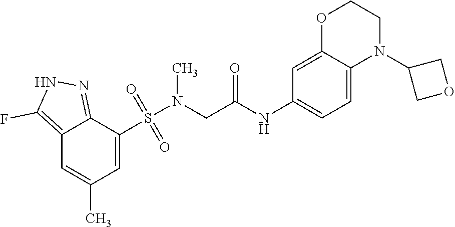 [SMILES: Cc1cc(S(=O)(=O)N(C)CC(=O)Nc2ccc3c(c2)OCCN3C2COC2)c2n[nH]c(F)c2c1] | 0.0278 | | 66 |
| 141 | 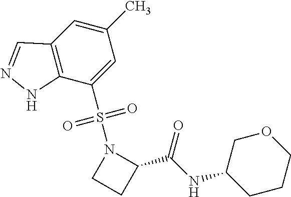 [SMILES: Cc1cc(S(=O)(=O)N2CC[C@H]2C(=O)N[C@H]2CCCOC2)c2[nH]ncc2c1] | 0.811 | | |
| 142 | 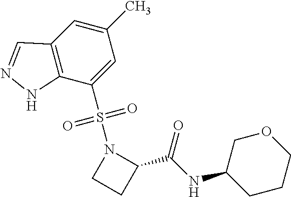 [SMILES: Cc1cc(S(=O)(=O)N2CC[C@H]2C(=O)N[C@@H]2CCCOC2)c2[nH]ncc2c1] | 0.47 | | |
| 143 | 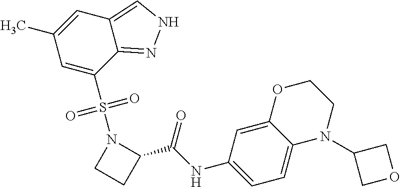 [SMILES: Cc1cc(S(=O)(=O)N2CC[C@H]2C(=O)Nc2ccc3c(c2)OCCN3C2COC2)c2n[nH]cc2c1] | 0.0362 | | 84 |
| 144 | 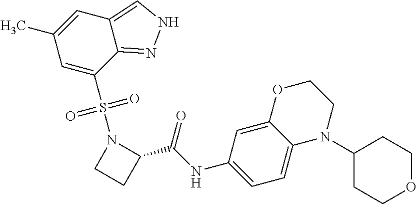 [SMILES: Cc1cc(S(=O)(=O)N2CC[C@H]2C(=O)Nc2ccc3c(c2)OCCN3C2CCOCC2)c2n[nH]cc2c1] | 0.0379 | | 166 |
| 145 | 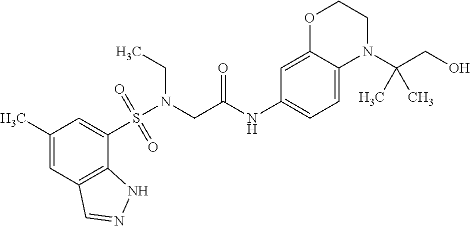 [SMILES: CCN(CC(=O)Nc1ccc2c(c1)OCCN2C(C)(C)CO)S(=O)(=O)c1cc(C)cc2cn[nH]c12] | 0.039 | | 92 |
| 146 | 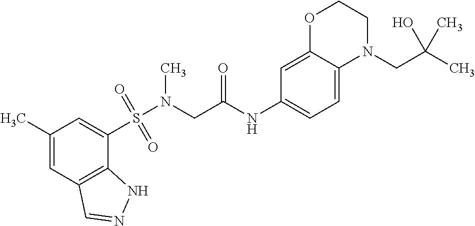 [SMILES: Cc1cc(S(=O)(=O)N(C)CC(=O)Nc2ccc3c(c2)OCCN3CC(C)(C)O)c2[nH]ncc2c1] | 0.026 | | 31 |
| 147 | 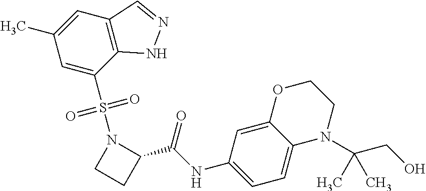 [SMILES: Cc1cc(S(=O)(=O)N2CC[C@H]2C(=O)Nc2ccc3c(c2)OCCN3C(C)(C)CO)c2[nH]ncc2c1] | 0.037 | | 212 |
| 148 | 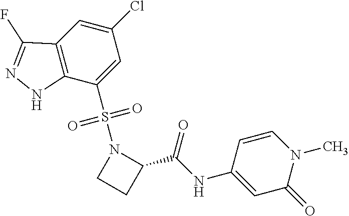 [SMILES: Cn1ccc(NC(=O)[C@@H]2CCN2S(=O)(=O)c2cc(Cl)cc3c(F)n[nH]c23)cc1=O] | 0.04 | | −17 |
| 149 | 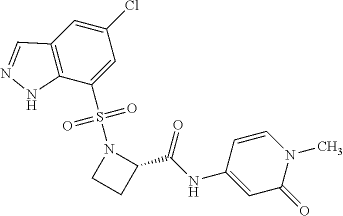 [SMILES: Cn1ccc(NC(=O)[C@@H]2CCN2S(=O)(=O)c2cc(Cl)cc3cn[nH]c23)cc1=O] | 0.061 | | 14 |
| 150 | 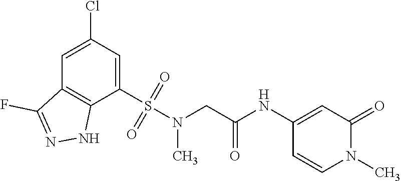 [SMILES: CN(CC(=O)Nc1ccn(C)c(=O)c1)S(=O)(=O)c1cc(Cl)cc2c(F)n[nH]c12] | 0.046 | | 1 |
| 151 | 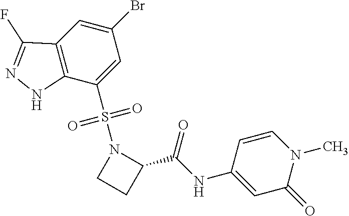 [SMILES: Cn1ccc(NC(=O)[C@@H]2CCN2S(=O)(=O)c2cc(Br)cc3c(F)n[nH]c23)cc1=O] | 0.18 | | −4 |
| 152 | 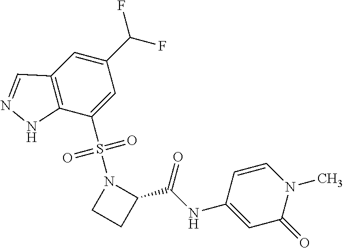 [SMILES: Cn1ccc(NC(=O)[C@@H]2CCN2S(=O)(=O)c2cc(C(F)F)cc3cn[nH]c23)cc1=O] | 0.058 | | −7 |
| 153 | 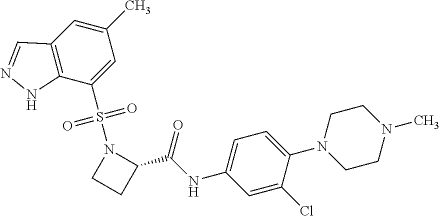 [SMILES: Cc1cc(S(=O)(=O)N2CC[C@H]2C(=O)Nc2ccc(N3CCN(C)CC3)c(Cl)c2)c2[nH]ncc2c1] | 0.048 | | 113 |
| 154 | 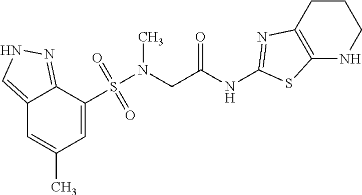 [SMILES: Cc1cc(S(=O)(=O)N(C)CC(=O)Nc2nc3c(s2)NCCC3)c2n[nH]cc2c1] | 0.05 | | |
| 155 | 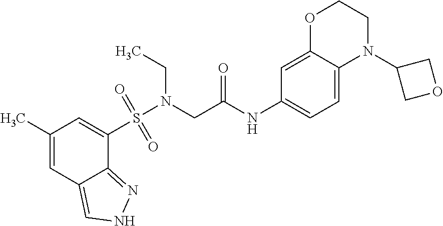 [SMILES: CCN(CC(=O)Nc1ccc2c(c1)OCCN2C1COC1)S(=O)(=O)c1cc(C)cc2c[nH]nc12] | 0.0246 | | |
| 156 | 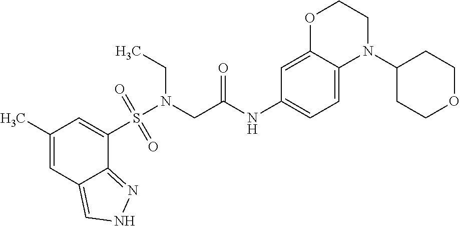 [SMILES: CCN(CC(=O)Nc1ccc2c(c1)OCCN2C1CCOCC1)S(=O)(=O)c1cc(C)cc2c[nH]nc12] | 0.0274 | | 58 |
| 157 | 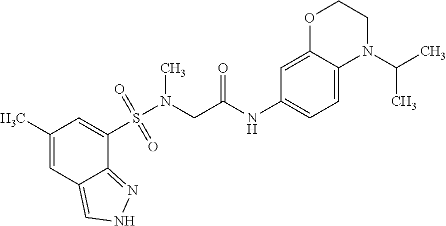 [SMILES: Cc1cc(S(=O)(=O)N(C)CC(=O)Nc2ccc3c(c2)OCCN3C(C)C)c2n[nH]cc2c1] | 0.014 | | |
| 158 | 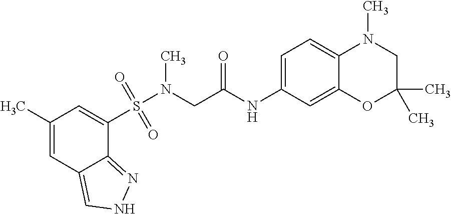 | 0.022 | | 46 |
| 159 | 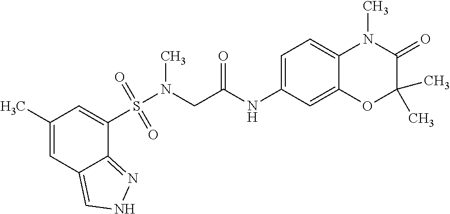 | 0.019 | | 67 |
| 160 | 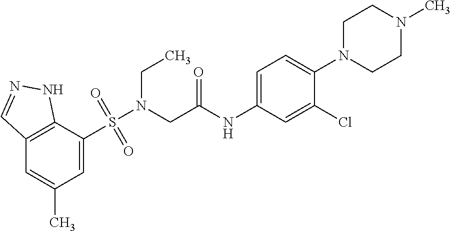 [SMILES: CCN(CC(=O)Nc1ccc(N2CCN(C)CC2)c(Cl)c1)S(=O)(=O)c1cc(C)cc2cn[nH]c12] | 0.106 | | 107 |
| 161 | 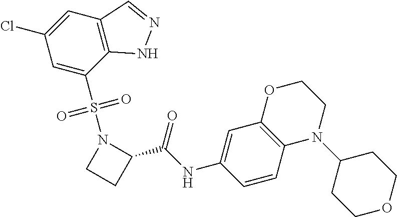 [SMILES: O=C(Nc1ccc2c(c1)OCCN2C1CCOCC1)[C@@H]1CCN1S(=O)(=O)c1cc(Cl)cc2cn[nH]c12] | 0.033 | | 121 |
| 162 | 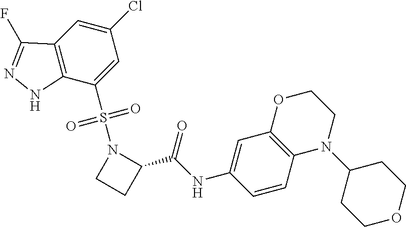 [SMILES: O=C(Nc1ccc2c(c1)OCCN2C1CCOCC1)[C@@H]1CCN1S(=O)(=O)c1cc(Cl)cc2c(F)n[nH]c12] | 0.059 | | 91 |
| 163 | 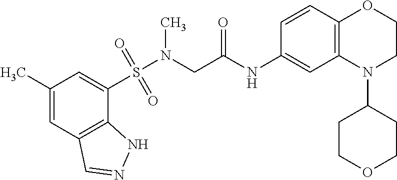 [SMILES: Cc1cc(S(=O)(=O)N(C)CC(=O)Nc2ccc3c(c2)N(C2CCOCC2)CCO3)c2[nH]ncc2c1] | 0.031 | | 94 |
| 164 | 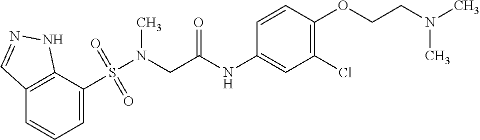 [SMILES: CN(C)CCOc1ccc(NC(=O)CN(C)S(=O)(=O)c2cccc3cn[nH]c23)cc1Cl] | 0.085 | | −16 |
| 165 | 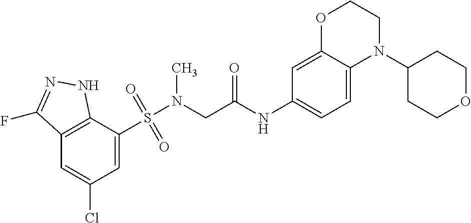 [SMILES: CN(CC(=O)Nc1ccc2c(c1)OCCN2C1CCOCC1)S(=O)(=O)c1cc(Cl)cc2c(F)n[nH]c12] | 0.037 | | 162 |
| 166 | 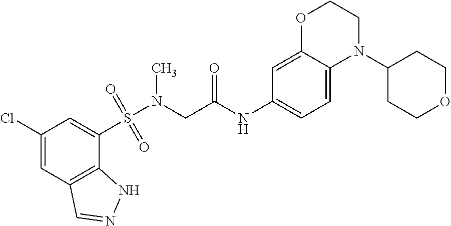 [SMILES: CN(CC(=O)Nc1ccc2c(c1)OCCN2C1CCOCC1)S(=O)(=O)c1cc(Cl)cc2cn[nH]c12] | 0.034 | | 122 |
| 167 | 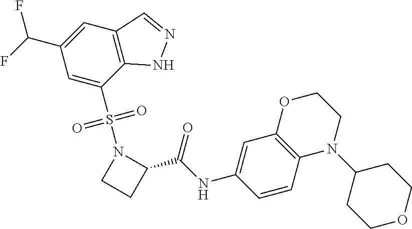 [SMILES: O=C(Nc1ccc2c(c1)OCCN2C1CCOCC1)[C@@H]1CCN1S(=O)(=O)c1cc(C(F)F)cc2cn[nH]c12] | 0.047 | | 118 |
| 168 | 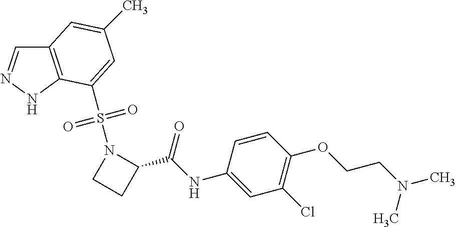 [SMILES: Cc1cc(S(=O)(=O)N2CC[C@H]2C(=O)Nc2ccc(OCCN(C)C)c(Cl)c2)c2[nH]ncc2c1] | 0.143 | | 1 |
| 169 | 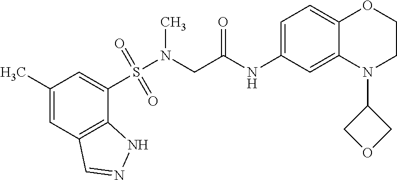 [SMILES: Cc1cc(S(=O)(=O)N(C)CC(=O)Nc2ccc3c(c2)N(C2COC2)CCO3)c2[nH]ncc2c1] | 0.038 | | 39 |
| 170 | 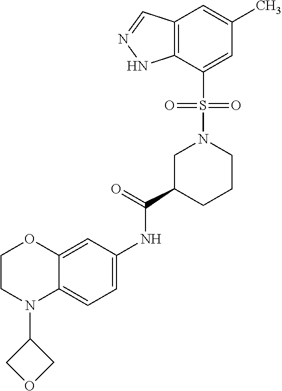 [SMILES: Cc1cc(S(=O)(=O)N2CCC[C@@H](C(=O)Nc3ccc4c(c3)OCCN4C3COC3)C2)c2[nH]ncc2c1] | 0.051 | | 34 |
| 171 | 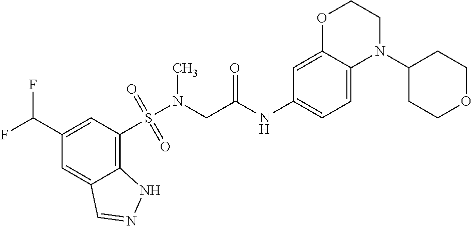 [SMILES: CN(CC(=O)Nc1ccc2c(c1)OCCN2C1CCOCC1)S(=O)(=O)c1cc(C(F)F)cc2cn[nH]c12] | 0.078 | | 102 |
| 172 | 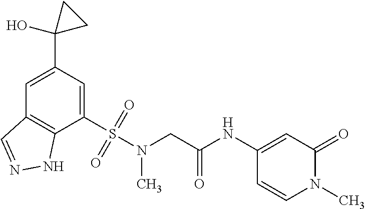 [SMILES: CN(CC(=O)Nc1ccn(C)c(=O)c1)S(=O)(=O)c1cc(C2(O)CC2)cc2cn[nH]c12] | 0.654 | | |
| 173 | 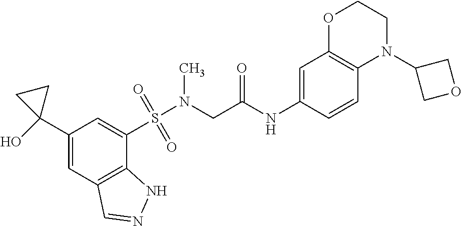 [SMILES: CN(CC(=O)Nc1ccc2c(c1)OCCN2C1COC1)S(=O)(=O)c1cc(C2(O)CC2)cc2cn[nH]c12] | 0.406 | | 60 |
| 174 | 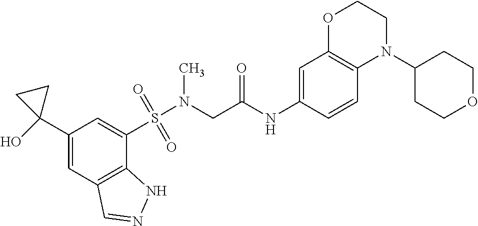 [SMILES: CN(CC(=O)Nc1ccc2c(c1)OCCN2C1CCOCC1)S(=O)(=O)c1cc(C2(O)CC2)cc2cn[nH]c12] | 0.55 | | 131 |
| 175 | 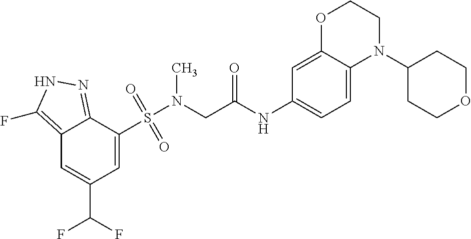 [SMILES: CN(CC(=O)Nc1ccc2c(c1)OCCN2C1CCOCC1)S(=O)(=O)c1cc(C(F)F)cc2c(F)[nH]nc12] | 0.089 | | 166 |
| 176 | 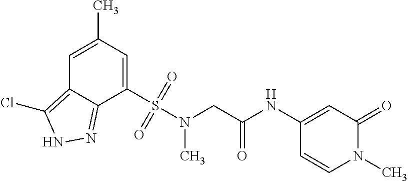 [SMILES: Cc1cc(S(=O)(=O)N(C)CC(=O)Nc2ccn(C)c(=O)c2)c2n[nH]c(Cl)c2c1] | 0.252 | | 34 |
| 177 | 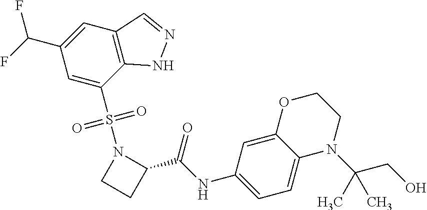 [SMILES: CC(C)(CO)N1CCOc2cc(NC(=O)[C@@H]3CCN3S(=O)(=O)c3cc(C(F)F)cc4cn[nH]c34)ccc21] | 0.197 | | 177 |
| 178 | 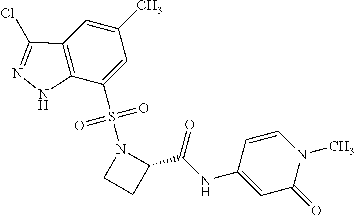 [SMILES: Cc1cc(S(=O)(=O)N2CC[C@H]2C(=O)Nc2ccn(C)c(=O)c2)c2[nH]nc(Cl)c2c1] | 0.211 | | −13 |
| 179 | 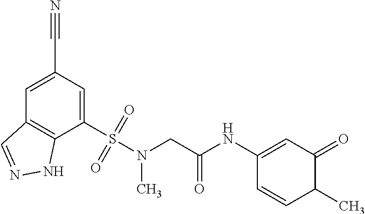 [SMILES: CC1C=CC(NC(=O)CN(C)S(=O)(=O)c2cc(C#N)cc3cn[nH]c23)=CC1=O] | 0.893 | | 0 |
| 180 | 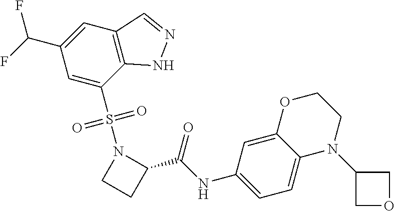 [SMILES: O=C(Nc1ccc2c(c1)OCCN2C1COC1)[C@@H]1CCN1S(=O)(=O)c1cc(C(F)F)cc2cn[nH]c12] | | | 61 |
| 181 | 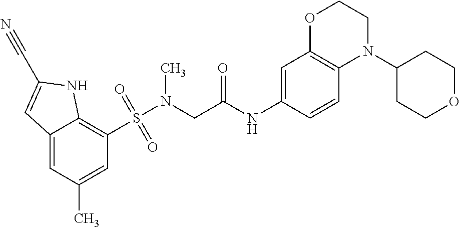 [SMILES: Cc1cc(S(=O)(=O)N(C)CC(=O)Nc2ccc3c(c2)OCCN3C2CCOCC2)c2[nH]c(C#N)cc2c1] | 0.095 | | 195 |
| 182 | 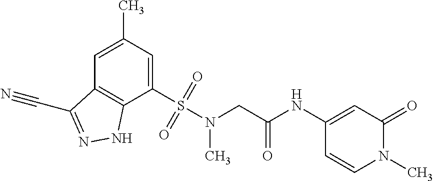 [SMILES: Cc1cc(S(=O)(=O)N(C)CC(=O)Nc2ccn(C)c(=O)c2)c2[nH]nc(C#N)c2c1] | 0.724 | | |
| 183 | 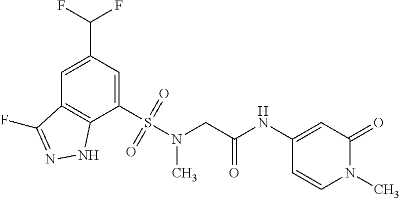 [SMILES: CN(CC(=O)Nc1ccn(C)c(=O)c1)S(=O)(=O)c1cc(C(F)F)cc2c(F)n[nH]c12] | 0.231 | | −15 |
| 184 | 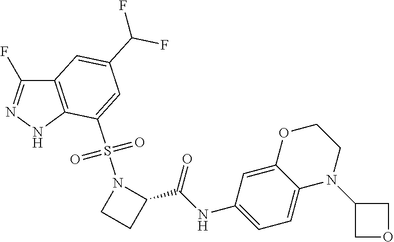 [SMILES: O=C(Nc1ccc2c(c1)OCCN2C1COC1)[C@@H]1CCN1S(=O)(=O)c1cc(C(F)F)cc2c(F)n[nH]c12] | 0.148 | | 36 |
| 185 | 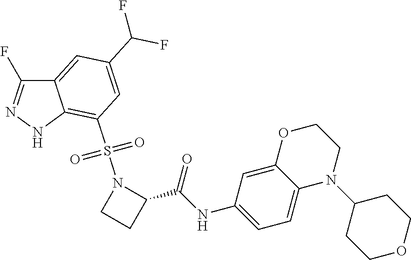 [SMILES: O=C(Nc1ccc2c(c1)OCCN2C1CCOCC1)[C@@H]1CCN1S(=O)(=O)c1cc(C(F)F)cc2c(F)n[nH]c12] | 0.183 | | 114 |
| 186 | 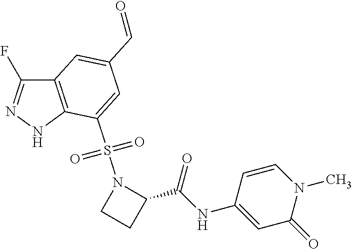 [SMILES: Cn1ccc(NC(=O)[C@@H]2CCN2S(=O)(=O)c2cc(C=O)cc3c(F)n[nH]c23)cc1=O] | 1.04 | | 153 |
| 187 | 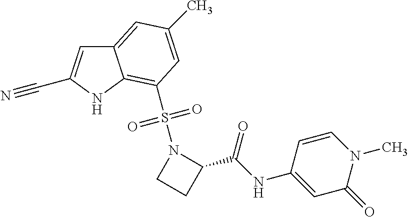 [SMILES: Cc1cc(S(=O)(=O)N2CC[C@H]2C(=O)Nc2ccn(C)c(=O)c2)c2[nH]c(C#N)cc2c1] | 0.135 | | −2 |
| 188 | 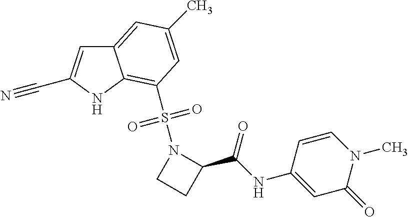 [SMILES: Cc1cc(S(=O)(=O)N2CC[C@@H]2C(=O)Nc2ccn(C)c(=O)c2)c2[nH]c(C#N)cc2c1] | 8.85 | | 5 |
| 189 | 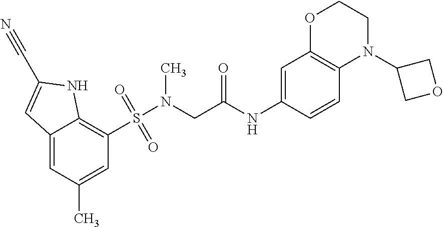 [SMILES: Cc1cc(S(=O)(=O)N(C)CC(=O)Nc2ccc3c(c2)OCCN3C2COC2)c2[nH]c(C#N)cc2c1] | 0.076 | 0.018 | 142 |
| 190 | 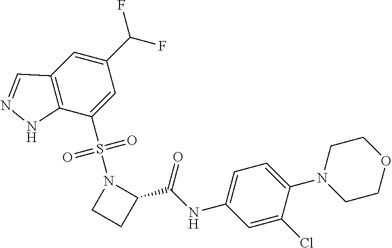 [SMILES: O=C(Nc1ccc(N2CCOCC2)c(Cl)c1)[C@@H]1CCN1S(=O)(=O)c1cc(C(F)F)cc2cn[nH]c12] | 0.111 | | 86 |
| 191 | 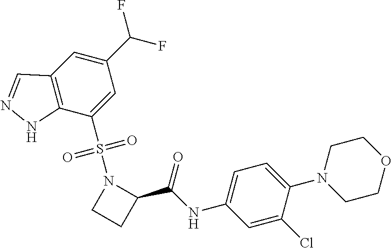 [SMILES: O=C(Nc1ccc(N2CCOCC2)c(Cl)c1)[C@H]1CCN1S(=O)(=O)c1cc(C(F)F)cc2cn[nH]c12] | >10 | | 98 |
| 192 | 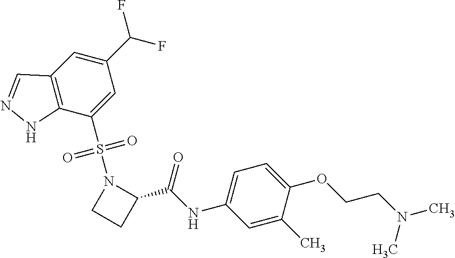 [SMILES: Cc1cc(NC(=O)[C@@H]2CCN2S(=O)(=O)c2cc(C(F)F)cc3cn[nH]c23)ccc1OCCN(C)C] | 0.102 | | 26 |
| 193 | 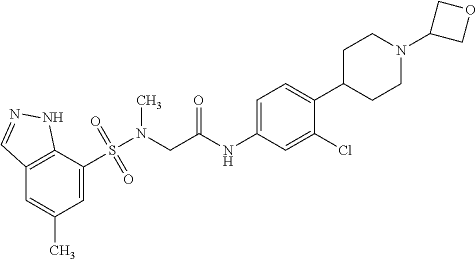 [SMILES: Cc1cc(S(=O)(=O)N(C)CC(=O)Nc2ccc(C3CCN(C4COC4)CC3)c(Cl)c2)c2[nH]ncc2c1] | 0.053 | | 73 |
| 194 | 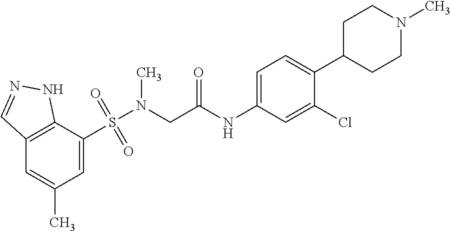 [SMILES: Cc1cc(S(=O)(=O)N(C)CC(=O)Nc2ccc(C3CCN(C)CC3)c(Cl)c2)c2[nH]ncc2c1] | 0.073 | | 35 |
| 195 | 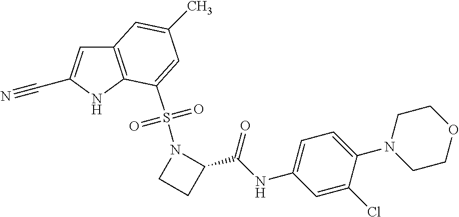 [SMILES: Cc1cc(S(=O)(=O)N2CC[C@H]2C(=O)Nc2ccc(N3CCOCC3)c(Cl)c2)c2[nH]c(C#N)cc2c1] | 0.111 | | 64 |
| 196 | 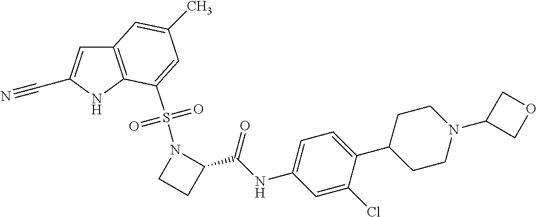 [SMILES: Cc1cc(S(=O)(=O)N2CC[C@H]2C(=O)Nc2ccc(C3CCN(C4COC4)CC3)c(Cl)c2)c2[nH]c(C#N)cc2c1] | 0.161 | | 161 |
| 197 | 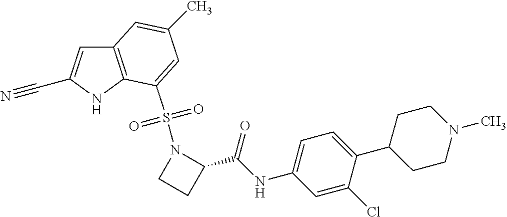 [SMILES: Cc1cc(S(=O)(=O)N2CC[C@H]2C(=O)Nc2ccc(C3CCN(C)CC3)c(Cl)c2)c2[nH]c(C#N)cc2c1] | 0.142 | | 80 |
| 198 | 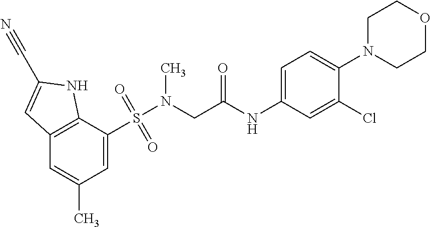 [SMILES: Cc1cc(S(=O)(=O)N(C)CC(=O)Nc2ccc(N3CCOCC3)c(Cl)c2)c2[nH]c(C#N)cc2c1] | 0.087 | | 137 |
| 199 | 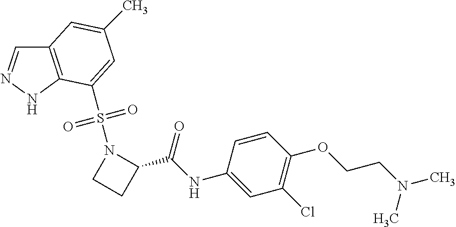 [SMILES: Cc1cc(S(=O)(=O)N2CC[C@H]2C(=O)Nc2ccc(OCCN(C)C)c(Cl)c2)c2[nH]ncc2c1] | 0.186 | 0.045 | −7 |
| 200 | 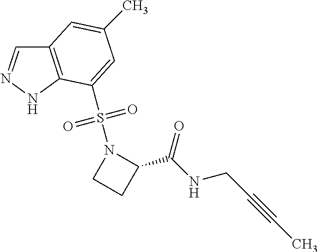 [SMILES: CC#CCNC(=O)[C@@H]1CCN1S(=O)(=O)c1cc(C)cc2cn[nH]c12] | 0.379 | | 13 |
| 201 | 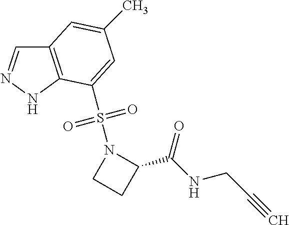 [SMILES: C#CCNC(=O)[C@@H]1CCN1S(=O)(=O)c1cc(C)cc2cn[nH]c12] | 0.222 | | 14 |
| 202 | 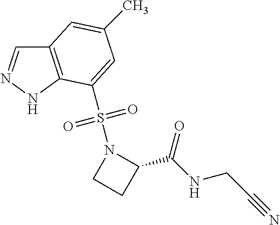 [SMILES: Cc1cc(S(=O)(=O)N2CC[C@H]2C(=O)NCC#N)c2[nH]ncc2c1] | 0.323 | | 4 |
| 203 | 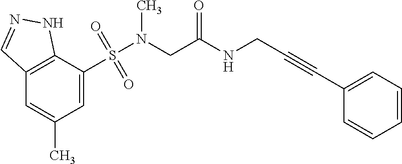 [SMILES: Cc1cc(S(=O)(=O)N(C)CC(=O)NCC#Cc2ccccc2)c2[nH]ncc2c1] | 0.043 | | 129 |
| 204 | 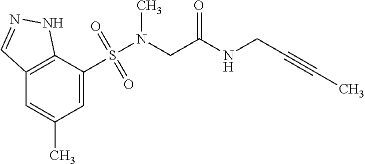 [SMILES: CC#CCNC(=O)CN(C)S(=O)(=O)c1cc(C)cc2cn[nH]c12] | 0.045 | 0.024 | 19 |
| 205 | 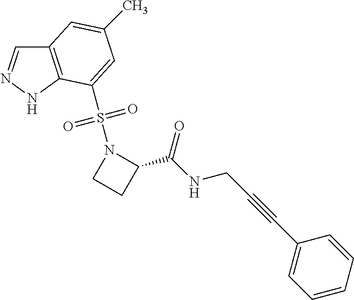 [SMILES: Cc1cc(S(=O)(=O)N2CC[C@H]2C(=O)NCC#Cc2ccccc2)c2[nH]ncc2c1] | 0.154 | | 205 |
| 206 | 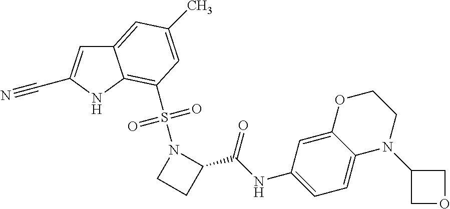 [SMILES: Cc1cc(S(=O)(=O)N2CC[C@H]2C(=O)Nc2ccc3c(c2)OCCN3C2COC2)c2[nH]c(C#N)cc2c1] | 0.081 | 0.022 | 111 |
| 207 | 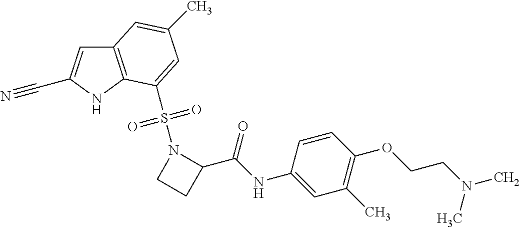 [SMILES: Cc1cc(S(=O)(=O)N2CCC2C(=O)Nc2ccc(OCCN(C)C)c(C)c2)c2[nH]c(C#N)cc2c1] | 0.225 | 0.06 | −7 |
| 208 | 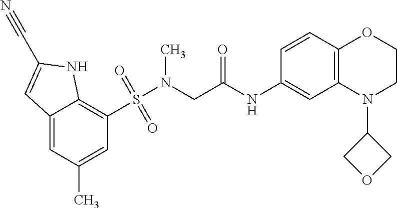 [SMILES: Cc1cc(S(=O)(=O)N(C)CC(=O)Nc2ccc3c(c2)N(C2COC2)CCO3)c2[nH]c(C#N)cc2c1] | | 0.015 | 341 |
| 209 | 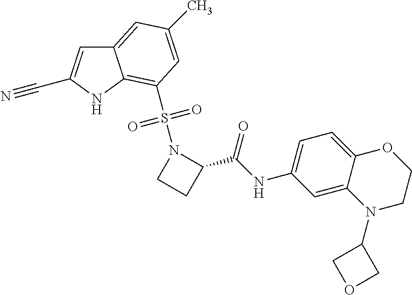 [SMILES: Cc1cc(S(=O)(=O)N2CC[C@H]2C(=O)Nc2ccc3c(c2)N(C2COC2)CCO3)c2[nH]c(C#N)cc2c1] | | 0.033 | 212 |
| 210 | 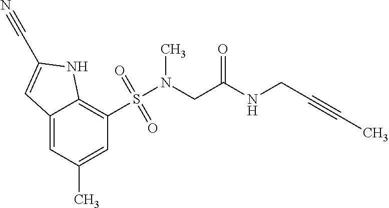 [SMILES: CC#CCNC(=O)CN(C)S(=O)(=O)c1cc(C)cc2cc(C#N)[nH]c12] | | 0.038 | 39 |
| 211 | 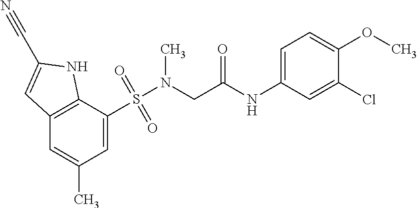 [SMILES: COc1ccc(NC(=O)CN(C)S(=O)(=O)c2cc(C)cc3cc(C#N)[nH]c23)cc1Cl] | | 0.02 | 358 |
| 212 | 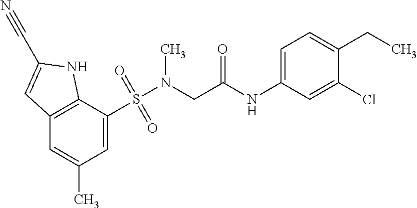 [SMILES: CCc1ccc(NC(=O)CN(C)S(=O)(=O)c2cc(C)cc3cc(C#N)[nH]c23)cc1Cl] | | 0.035 | 210 |
| 213 | 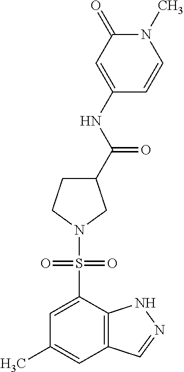 | 1.92 | | |
| 214 | 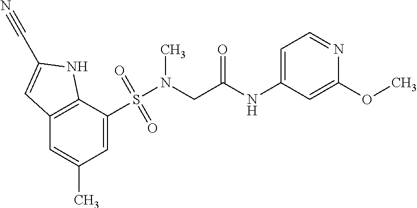 [SMILES: COc1cc(NC(=O)CN(C)S(=O)(=O)c2cc(C)cc3cc(C#N)[nH]c23)ccn1] | | 0.021 | 91 |
| 215 | 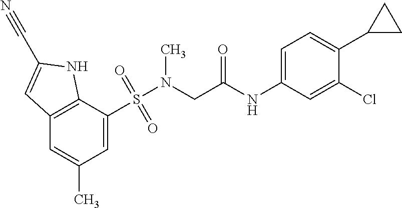 [SMILES: Cc1cc(S(=O)(=O)N(C)CC(=O)Nc2ccc(C3CC3)c(Cl)c2)c2[nH]c(C#N)cc2c1] | | 0.036 | 203 |
| 216 | 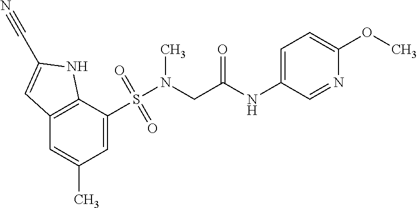 [SMILES: COc1ccc(NC(=O)CN(C)S(=O)(=O)c2cc(C)cc3cc(C#N)[nH]c23)cn1] | | 0.02 | 49 |
| 217 | 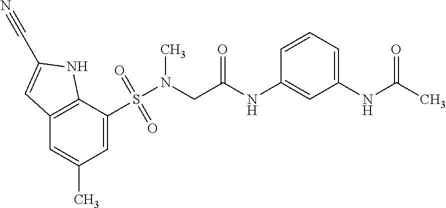 [SMILES: CC(=O)Nc1cccc(NC(=O)CN(C)S(=O)(=O)c2cc(C)cc3cc(C#N)[nH]c23)c1] | | 0.028 | 177 |
| 218 | 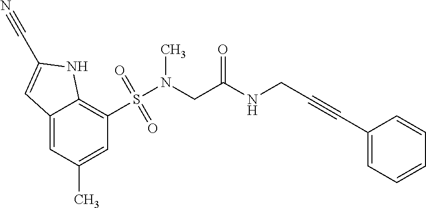 [SMILES: Cc1cc(S(=O)(=O)N(C)CC(=O)NCC#Cc2ccccc2)c2[nH]c(C#N)cc2c1] | | 0.03 | 191 |
| 219 | 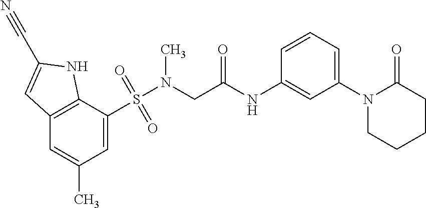 [SMILES: Cc1cc(S(=O)(=O)N(C)CC(=O)Nc2cccc(N3CCCCC3=O)c2)c2[nH]c(C#N)cc2c1] | | 0.03 | 150 |
| 220 | 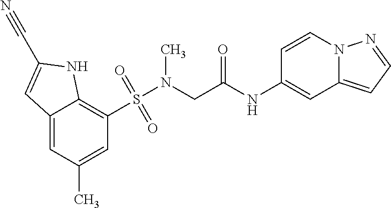 [SMILES: Cc1cc(S(=O)(=O)N(C)CC(=O)Nc2ccn3nccc3c2)c2[nH]c(C#N)cc2c1] | | 0.019 | |
| 221 | 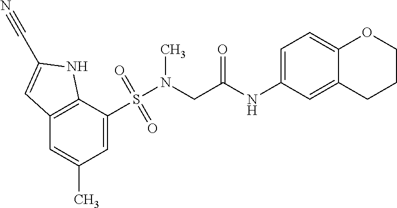 [SMILES: Cc1cc(S(=O)(=O)N(C)CC(=O)Nc2ccc3c(c2)CCCO3)c2[nH]c(C#N)cc2c1] | | 0.015 | |
| 222 | 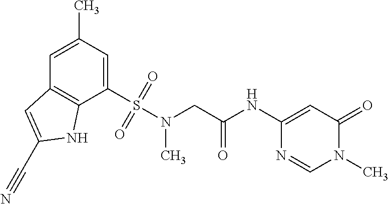 [SMILES: Cc1cc(S(=O)(=O)N(C)CC(=O)Nc2cc(=O)n(C)cn2)c2[nH]c(C#N)cc2c1] | | 0.027 | |
| 223 | 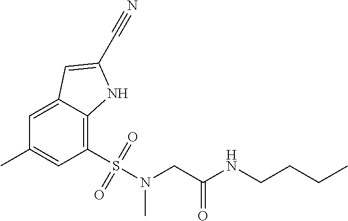 [SMILES: CCCCNC(=O)CN(C)S(=O)(=O)c1cc(C)cc2cc(C#N)[nH]c12] | | 0.028 | 62 |
| 224 | 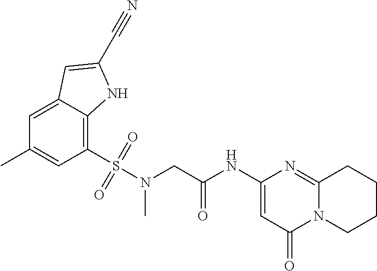 [SMILES: Cc1cc(S(=O)(=O)N(C)CC(=O)Nc2cc(=O)n3c(n2)CCCC3)c2[nH]c(C#N)cc2c1] | | 0.022 | 61 |
| 225 | 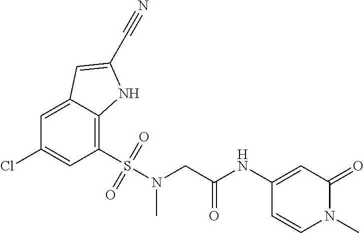 [SMILES: CN(CC(=O)Nc1ccn(C)c(=O)c1)S(=O)(=O)c1cc(Cl)cc2cc(C#N)[nH]c12] | | 0.035 | 11 |
| 226 | 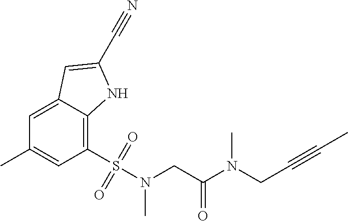 [SMILES: CC#CCN(C)C(=O)CN(C)S(=O)(=O)c1cc(C)cc2cc(C#N)[nH]c12] | | 0.368 | |
| 227 | 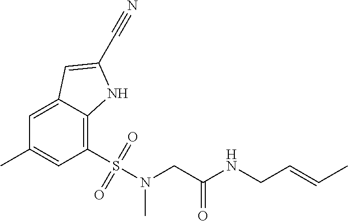 [SMILES: C/C=C/CNC(=O)CN(C)S(=O)(=O)c1cc(C)cc2cc(C#N)[nH]c12] | | 0.018 | |
| 228 | 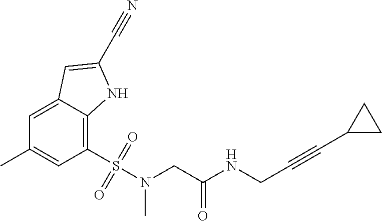 [SMILES: Cc1cc(S(=O)(=O)N(C)CC(=O)NCC#CC2CC2)c2[nH]c(C#N)cc2c1] | | | |
| 229 | 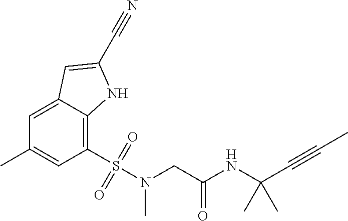 [SMILES: CC#CC(C)(C)NC(=O)CN(C)S(=O)(=O)c1cc(C)cc2cc(C#N)[nH]c12] | | 0.031 | 160 |

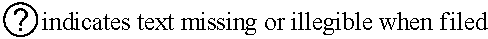

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of formula I: 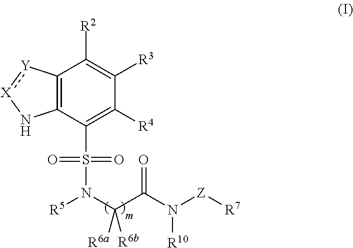
or a pharmaceutically acceptable salt thereof, wherein:
R², R³, and R⁴ independently are hydrogen, halogen, amino, hydroxyl, alkoxy, cyano, nitro, carbonyl, alkyl, alkenyl, alkynyl, cycloalkyl, heterocyclyl, aryl, or heteroaryl;
R⁵ and R¹⁰ are independently hydrogen, alkyl, cycloalkyl, heterocyclyl, aryl, or heteroaryl;
each R^{6a} and R^{6b} independently is hydrogen, halogen, amino, hydroxyl, alkoxy, cyano, nitro, alkyl, alkenyl, alkynl, cycloalkyl, heterocyclyl, aryl, or heteroaryl; or R^{6a} and R^{6b} complete a cycloalkyl, heterocyclyl, or an oxo group; or R⁵, R^{6a}, and the intervening carbon and nitrogen atoms complete a 3- to 6-membered heterocyclyl;
R⁷ is alkyl, alkenyl, alkynyl, cycloalkyl, heterocyclyl, aryl, or heteroaryl; [symbol]
is a single bond or a double bond; wherein when [symbol] is a double bond, X is N or CR⁸, and Y is N or CR¹; when [symbol] is a single bond, X is NR⁵, C(R⁸)₂, or C(═O) and Y is NR⁵, C(R¹)₂, or C(═O);
R¹ and R⁸ independently are hydrogen, halogen, amino, hydroxyl, alkoxy, cyano, nitro, alkyl, alkenyl, alkynyl, cycloalkyl, heterocyclyl, aryl, or heteroaryl;
Z is —C(R⁹)₂— or a bond;
each R⁹ is independently H or alkyl; and
m is an integer from 1 to 3.

2. The compound of claim 1, wherein the compound is of formula I: 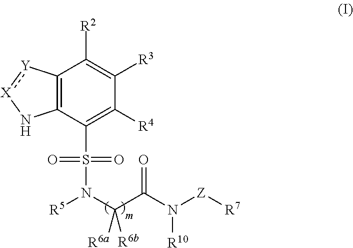
or a pharmaceutically acceptable salt thereof, wherein:
R², R³, and R⁴ independently are hydrogen, halogen, amino, hydroxyl, alkoxy, cyano, nitro, alkyl, alkenyl, alkynl, cycloalkyl, heterocyclyl, aryl, or heteroaryl;
R⁵ and R¹⁰ are independently hydrogen, alkyl, cycloalkyl, heterocyclyl, aryl, or heteroaryl;
each R^{6a} and R^{6b} independently is hydrogen, halogen, amino, hydroxyl, alkoxy, cyano, nitro, alkyl, alkenyl, alkynl, cycloalkyl, heterocyclyl, aryl, or heteroaryl; or R^{6a} and R^{6b} complete a cycloalkyl, heterocyclyl, or an oxo group; or R⁵, R^{6a}, and the intervening carbon and nitrogen atoms complete a 3- to 6-membered heterocyclyl;
R⁷ is alkyl, cycloalkyl, heterocyclyl, aryl, or heteroaryl; [symbol]
is a single bond or a double bond; wherein when [symbol] is a double bond, X is N or CR⁸, and Y is N or CR¹; when [symbol] is a single bond, X is NR⁵, C(R⁸)₂, or C(═O) and Y is NR⁵, C(R¹)₂, or C(═O);
R¹ and R⁸ independently are hydrogen, halogen, amino, hydroxyl, alkoxy, cyano, nitro, alkyl, alkenyl, alkynl, cycloalkyl, heterocyclyl, aryl, or heteroaryl;
Z is CH₂ or a bond; and
m is an integer from 1 to 3.

3. The compound of claim 1 or 2, wherein the compound is a compound of formula Ia 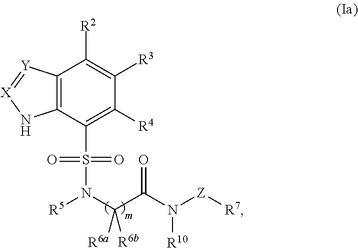
or a pharmaceutically acceptable salt thereof.

4. The compound of any one of claims 1-3, wherein X is CR⁸.

5. The compound of claim 4, wherein R⁸ is hydrogen, halogen, amino, alkoxy, cyano, nitro, alkyl, cycloalkyl, or heterocyclyl.

6. The compound of claim 4, wherein R⁸ is hydrogen, halogen, cyano, or C₁₋₆ alkyl.

7. The compound of claim 4, wherein R⁸ is hydrogen, cyano, or methyl.

8. The compound of any one of claims 1-3, wherein X is N.

9. The compound of any one of claims of 1-8, wherein Y is CR¹.

10. The compound of claim 9, wherein R¹ is hydrogen, halogen, alkyl, alkenyl, alkynl, cycloalkyl, heterocyclyl, aryl, or heteroaryl.

11. The compound of claim 9, wherein R¹ is hydrogen, halogen, or C₁₋₆ alkyl.

12. The compound of claim 9, wherein R¹ is hydrogen, methyl, halogen, cyano, methylamino methyl, or methoxy methyl.

13. The compound of claim 9, wherein R¹ is hydrogen.

14. The compound of any one of claims 1-8, wherein Y is N.

15. The compound of any one of claims 1-9, wherein the compound is a compound of formula IIa 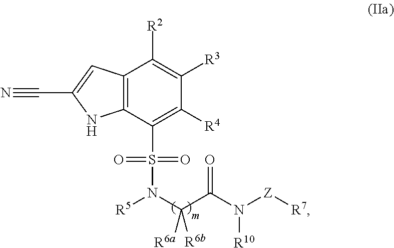
or a pharmaceutically acceptable salt thereof.

16. The compound of any one of claims 1-15, wherein R² is hydrogen, halogen, alkyl, alkenyl, alkynl, cycloalkyl, heterocyclyl, aryl, or heteroaryl.

17. The compound of any one of claims 1-15, wherein R² is hydrogen, halogen, or C₁₋₆ alkyl.

18. The compound of any one of claims 1-15, wherein R² is hydrogen or fluoro.

19. The compound of any one of claims 1-18, wherein R³ is halogen, cyano, alkyl, cycloalkyl, or heterocyclyl.

20. The compound of any one of claims 1-18, wherein R³ is halogen, cyano, C₁₋₆ alkyl, C₃₋₁₀ cycloalkyl, or C₁₋₆ haloalkyl.

21. The compound of any one of claims 1-18, wherein R³ is cyano, methyl, chloro, bromo, cyclopropyl, or difluoromethyl.

22. The compound of any one of claims 1-21, wherein R⁴ is hydrogen, halogen, alkyl, cycloalkyl, or heterocyclyl.

23. The compound of any one of claims 1-22, wherein R⁴ is hydrogen or halogen.

24. The compound of any one of claims 1-23, wherein R⁴ is hydrogen or fluoro.

25. The compound of any one of claims 1-24, wherein the compound of formula I is a compound of formula I-1 or I-2 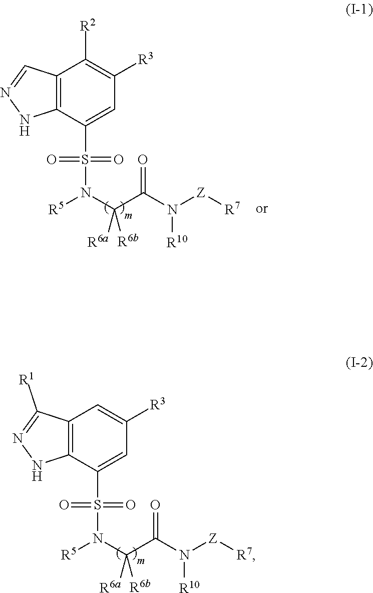
or a pharmaceutically acceptable salt thereof, wherein m is 1 or 2.

26. The compound of any one of claims 1-25, wherein R⁵ is hydrogen, alkyl, cycloalkyl, or heterocyclyl.

27. The compound of any one of claims 1-26, wherein R⁵ is C₁₋₆ alkyl or 3- to 10-membered heterocyclyl.

28. The compound of any one of claims 1-27, wherein R⁵ is methyl, ethyl, isopropyl, or oxetanyl.

29. The compound of any one of claims 1-28, wherein R^{6a} and R^{6b} independently are hydrogen or alkyl.

30. The compound of any one of claims 1-28, wherein R^{6a} and R^{6b} are taken together to form a C₃₋₁₀ carbocycle, a 3- to 10-membered heterocycle, or an oxo group.

31. The compound of any one of claims 1-25, wherein R⁵, R^{6a}, and the intervening carbon and nitrogen atoms are taken together to form a 3- to 6-membered heterocycle.

32. The compound of any one of claims 1-31, wherein m is 1 or 2, such as 1.

33. The compound of any one of claims 1-32, wherein the compound of formula I is a compound of formula I-1-a, I-1-b, I-1-c, I-2-a, I-2-b, or I-2-c: 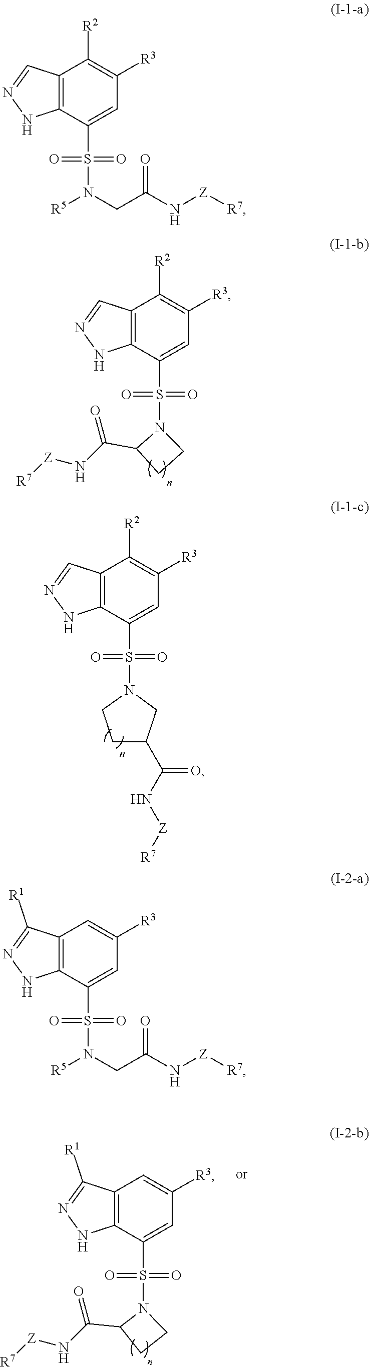 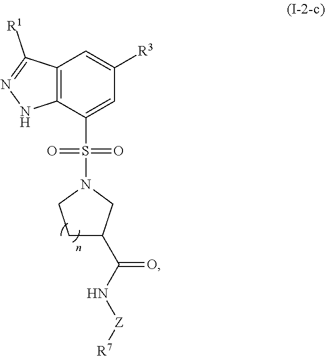
or a pharmaceutically acceptable salt thereof, wherein n is 1 or 2.

34. The compound of any one of claims of 1-28, wherein the compound of formula I is a compound of formula I-3 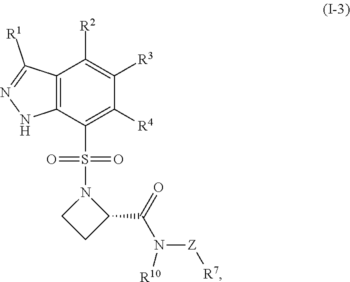
or a pharmaceutically acceptable salt thereof.

35. The compound of any one of claims of 1-28, wherein the compound of formula I is a compound of formula II-1-a 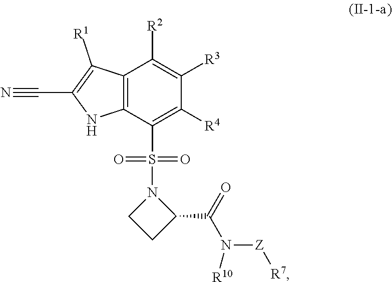
or a pharmaceutically acceptable salt thereof.

36. The compound of any one of claims of 1-35, wherein Z is a bond.

37. The compound of any one of claims of 1-35, wherein Z is CH₂.

38. The compound of any one of claims 1-37, wherein R⁷ is alkyl, alkenyl, or alkynyl.

39. The compound of claim 38, wherein the alkyl, alkenyl, or alkynyl is substituted with aryl or cycloalkyl.

40. The compound of any one of claims 1-37, wherein R⁷ is C₁-C₆ alkyl, C₂-C₆ alkenyl, or C₂-C₆ alkynyl.

41. The compound of any one of claims 1-37, wherein R⁷ is C₁₋₆ alkyl, C₃₋₁₀ cycloalkyl or 3- to 10-membered heterocyclyl, C₆₋₁₀ aryl or 5- to 10-membered heteroaryl.

42. The compound of any one of claims 1-37, wherein R⁷ is C₁₋₆ alkyl, C₆₋₁₀ aryl or 5- to 10-membered heterocyclyl optionally substituted with C₁₋₃ alkyl, cyclopropyl, C₁₋₃ alkoxy, halogen, oxo, cyano, phenyl, morpholino, piperidine, tetrahydropyran, or oxetane.

43. The compound of any one of claims 1-37, wherein R⁷ is cyclohexyl, tetrahydropyran, piperidine, piperidin-2-one, phenyl, pyridyl, pyridine-2-one, thiazole, oxazole, triazole, benzoxazole, benzthiazole, quinoline, 3,4-dihydro-2H-benzo[b][1,4]oxazine, 2,3-dihydrobenzo[b][1,4]dioxine, 4,5,6,7-tetrahydrothiazolo[5,4-c]pyridine, 4,5,6,7-tetrahydrothiazolo[4,5-c]pyridine, 2,3-dihydro-1H-indene, 6,7-dihydro-5H-cyclopenta[c]pyridine, imidazo[1,5-a]pyridine, [1,2,4]triazolo[4,3-a]pyridine, pyrazolo[1,5-a]pyridine, pyrazolo[1,5-a]pyridine, indolin-2-one.

44. The compound of any one of claims 1-37, wherein R⁷ is

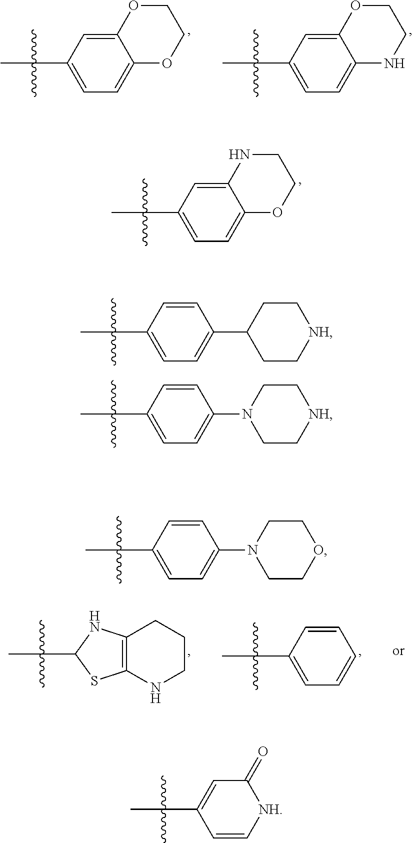

45. The compound of any one of claims 1-44, wherein R⁷ is optionally substituted with C₁₋₆ alkyl, cyclopropyl, C₁₋₃ alkoxy, halogen, oxo, cyano, phenyl, amido, or heterocyclyl such as morpholino, piperidine, piperazine, piperidin-2-one, tetrahydropyran, and oxetane.

46. The compound of any one of claims 1-45, wherein R¹⁰ is hydrogen.

47. The compound of claim 1, wherein the compound is selected from 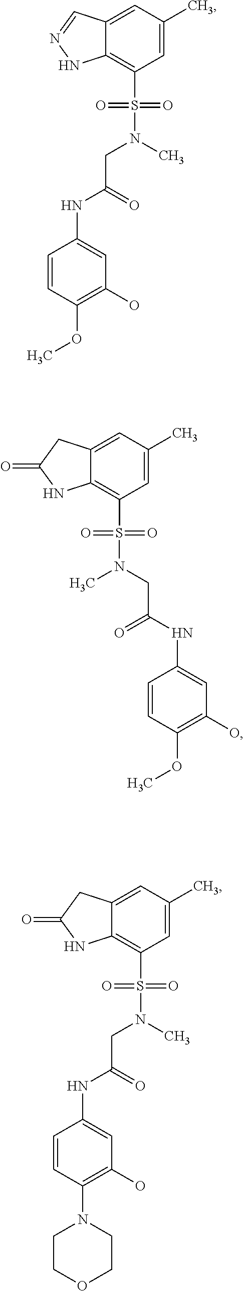 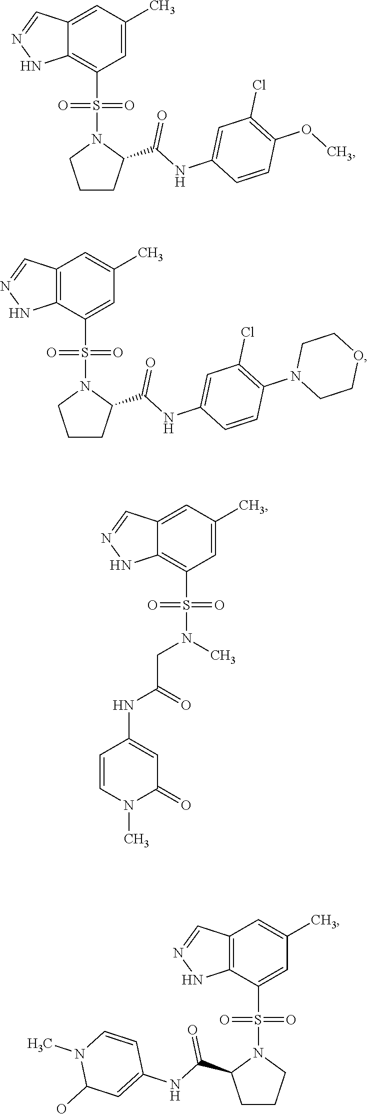 [SMILES: COc1ccc(NC(=O)[C@@H]2CCCN2S(=O)(=O)c2cc(C)cc3cn[nH]c23)cc1Cl ; Cc1cc(S(=O)(=O)N2CCC[C@H]2C(=O)Nc2ccc(N3CCOCC3)c(Cl)c2)c2[nH]ncc2c1 ; Cc1cc(S(=O)(=O)N(C)CC(=O)Nc2ccn(C)c(=O)c2)c2[nH]ncc2c1 ; Cc1cc(S(=O)(=O)N2CCC[C@H]2C(=O)NC2=CC(O)N(C)C=C2)c2[nH]ncc2c1] 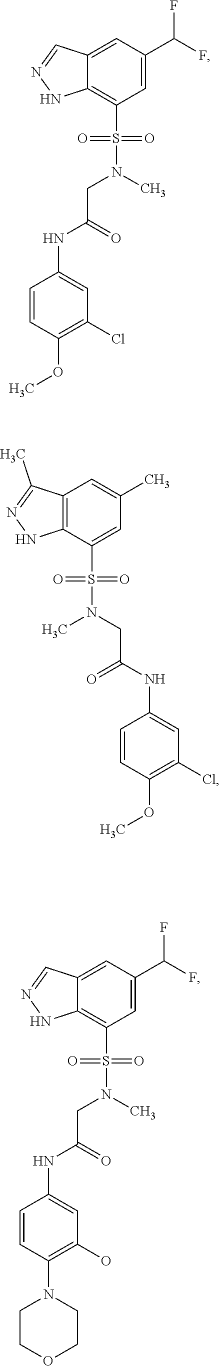 [SMILES: COc1ccc(NC(=O)CN(C)S(=O)(=O)c2cc(C(F)F)cc3cn[nH]c23)cc1Cl ; COc1ccc(NC(=O)CN(C)S(=O)(=O)c2cc(C)cc3c(C)n[nH]c23)cc1Cl ; CN(CC(=O)Nc1ccc(N2CCOCC2)c(O)c1)S(=O)(=O)c1cc(C(F)F)cc2cn[nH]c12] 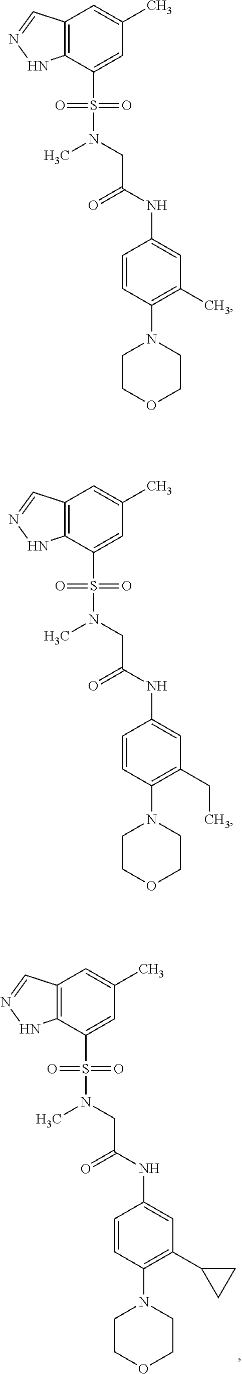 [SMILES: Cc1cc(S(=O)(=O)N(C)CC(=O)Nc2ccc(N3CCOCC3)c(C)c2)c2[nH]ncc2c1 ; CCc1cc(NC(=O)CN(C)S(=O)(=O)c2cc(C)cc3cn[nH]c23)ccc1N1CCOCC1 ; Cc1cc(S(=O)(=O)N(C)CC(=O)Nc2ccc(N3CCOCC3)c(C3CC3)c2)c2[nH]ncc2c1] 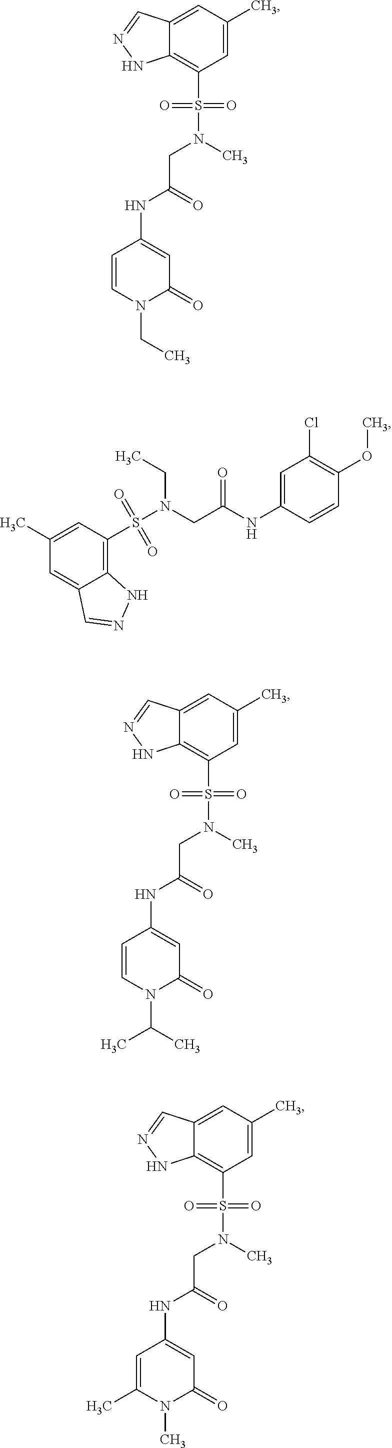 [SMILES: CCn1ccc(NC(=O)CN(C)S(=O)(=O)c2cc(C)cc3cn[nH]c23)cc1=O ; CCN(CC(=O)Nc1ccc(OC)c(Cl)c1)S(=O)(=O)c1cc(C)cc2cn[nH]c12 ; Cc1cc(S(=O)(=O)N(C)CC(=O)Nc2ccn(C(C)C)c(=O)c2)c2[nH]ncc2c1 ; Cc1cc(S(=O)(=O)N(C)CC(=O)Nc2cc(C)n(C)c(=O)c2)c2[nH]ncc2c1] 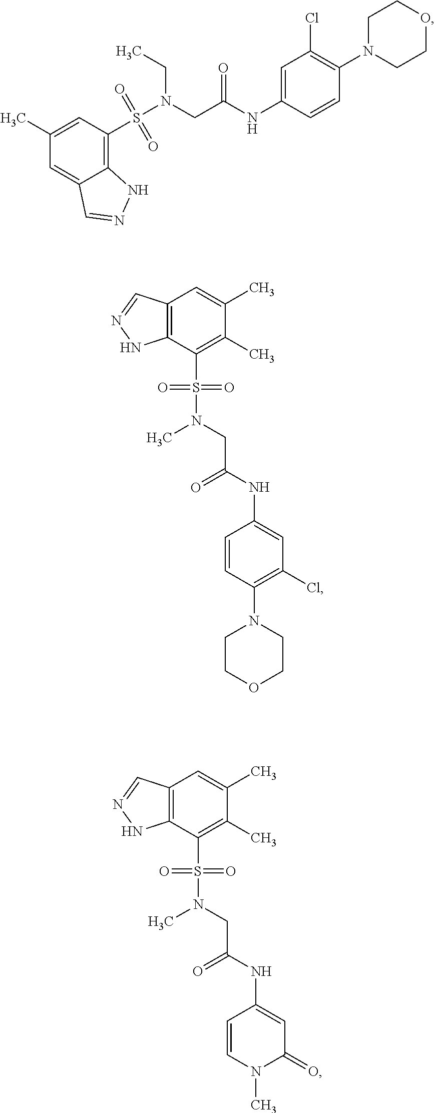 [SMILES: CCN(CC(=O)Nc1ccc(N2CCOCC2)c(Cl)c1)S(=O)(=O)c1cc(C)cc2cn[nH]c12 ; Cc1cc2cn[nH]c2c(S(=O)(=O)N(C)CC(=O)Nc2ccc(N3CCOCC3)c(Cl)c2)c1C ; Cc1cc2cn[nH]c2c(S(=O)(=O)N(C)CC(=O)Nc2ccn(C)c(=O)c2)c1C] 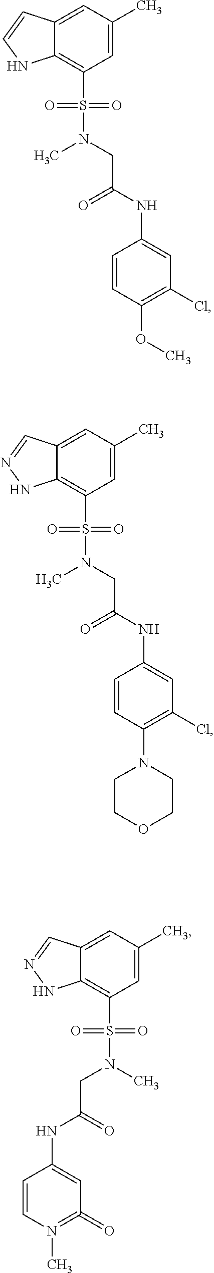 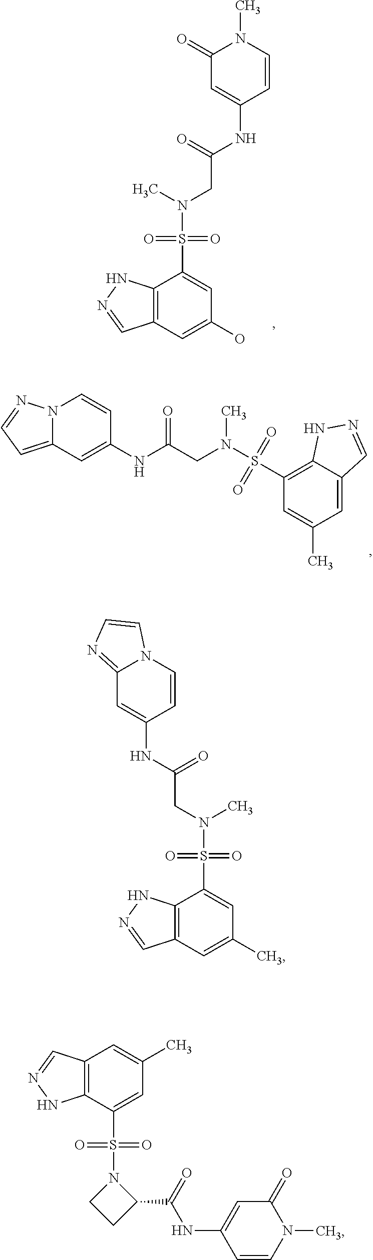 [SMILES: CN(CC(=O)Nc1ccn(C)c(=O)c1)S(=O)(=O)c1cc(O)cc2cn[nH]c12 ; Cc1cc(S(=O)(=O)N(C)CC(=O)Nc2ccn3nccc3c2)c2[nH]ncc2c1 ; Cc1cc(S(=O)(=O)N(C)CC(=O)Nc2ccn3ccnc3c2)c2[nH]ncc2c1 ; Cc1cc(S(=O)(=O)N2CC[C@H]2C(=O)Nc2ccn(C)c(=O)c2)c2[nH]ncc2c1] 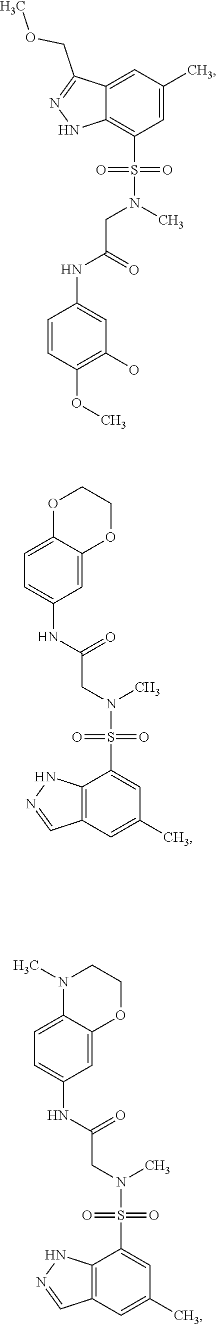 [SMILES: COCc1n[nH]c2c(S(=O)(=O)N(C)CC(=O)Nc3ccc(OC)c(O)c3)cc(C)cc12 ; Cc1cc(S(=O)(=O)N(C)CC(=O)Nc2ccc3c(c2)OCCO3)c2[nH]ncc2c1 ; Cc1cc(S(=O)(=O)N(C)CC(=O)Nc2ccc3c(c2)OCCN3C)c2[nH]ncc2c1] 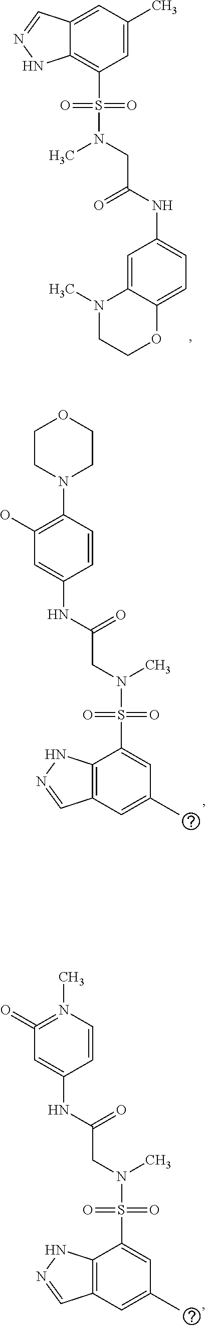 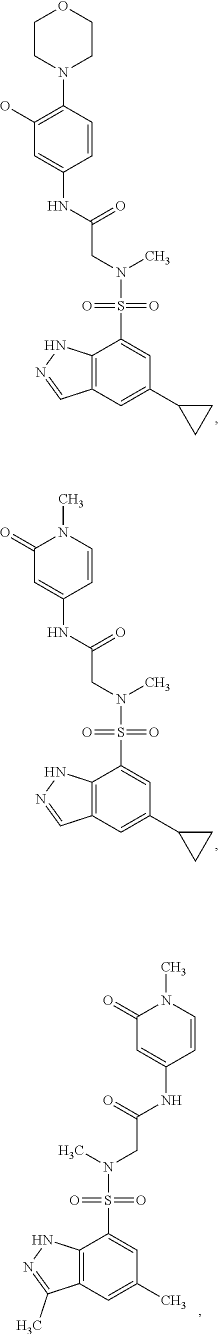 [SMILES: CN(CC(=O)Nc1ccc(N2CCOCC2)c(O)c1)S(=O)(=O)c1cc(C2CC2)cc2cn[nH]c12 ; CN(CC(=O)Nc1ccn(C)c(=O)c1)S(=O)(=O)c1cc(C2CC2)cc2cn[nH]c12 ; Cc1cc(S(=O)(=O)N(C)CC(=O)Nc2ccn(C)c(=O)c2)c2[nH]nc(C)c2c1] 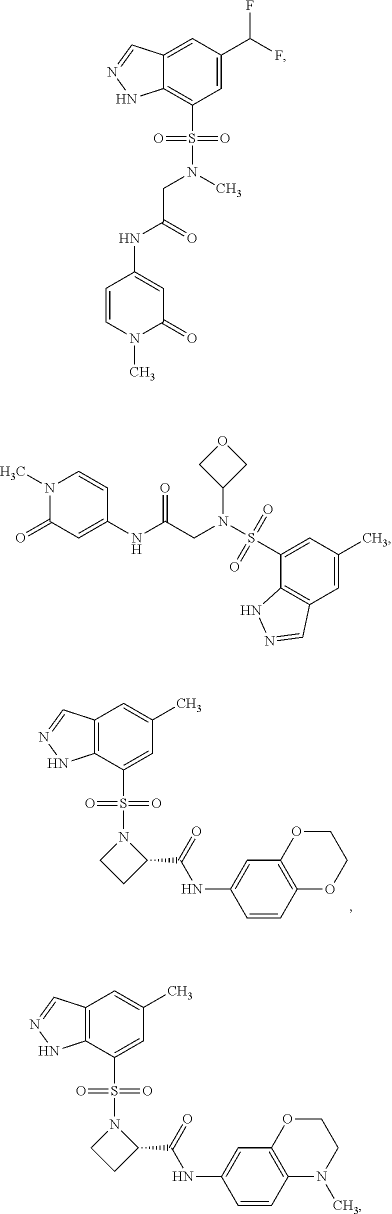 [SMILES: CN(CC(=O)Nc1ccn(C)c(=O)c1)S(=O)(=O)c1cc(C(F)F)cc2cn[nH]c12 ; Cc1cc(S(=O)(=O)N(CC(=O)Nc2ccn(C)c(=O)c2)C2COC2)c2[nH]ncc2c1 ; Cc1cc(S(=O)(=O)N2CC[C@H]2C(=O)Nc2ccc3c(c2)OCCO3)c2[nH]ncc2c1 ; Cc1cc(S(=O)(=O)N2CC[C@H]2C(=O)Nc2ccc3c(c2)OCCN3C)c2[nH]ncc2c1] 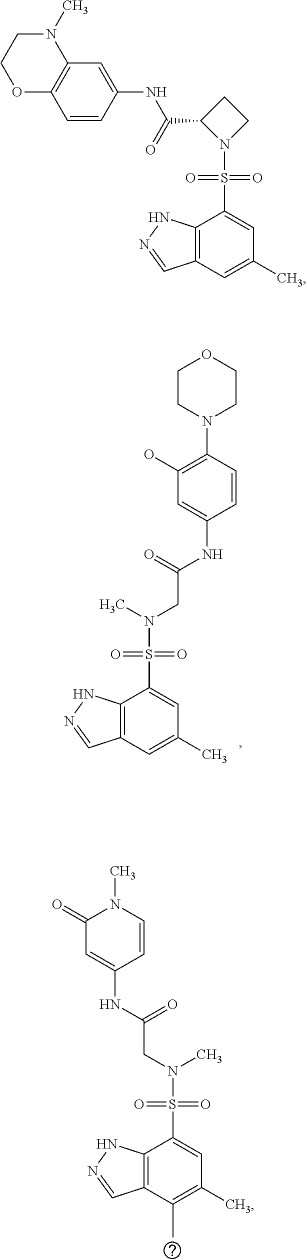 [SMILES: Cc1cc(S(=O)(=O)N2CC[C@H]2C(=O)Nc2ccc3c(c2)N(C)CCO3)c2[nH]ncc2c1 ; Cc1cc(S(=O)(=O)N(C)CC(=O)Nc2ccc(N3CCOCC3)c(O)c2)c2[nH]ncc2c1 ; Cc1cc(S(=O)(=O)N(C)CC(=O)Nc2ccn(C)c(=O)c2)c2[nH]ncc2c1C] 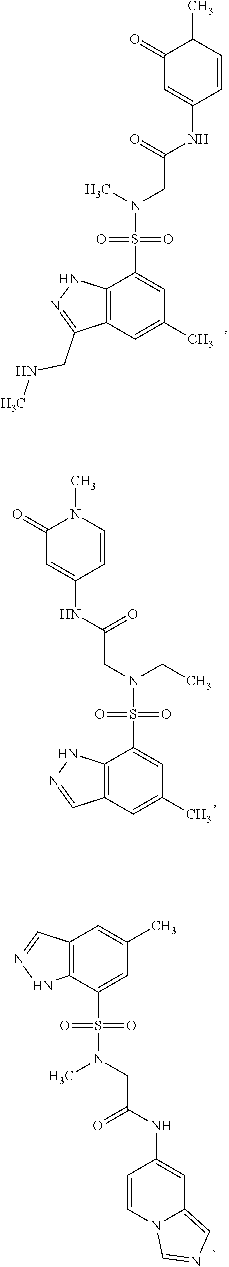 [SMILES: CNCc1n[nH]c2c(S(=O)(=O)N(C)CC(=O)NC3=CC(=O)C(C)C=C3)cc(C)cc12 ; CCN(CC(=O)Nc1ccn(C)c(=O)c1)S(=O)(=O)c1cc(C)cc2cn[nH]c12 ; Cc1cc(S(=O)(=O)N(C)CC(=O)Nc2ccn3cncc3c2)c2[nH]ncc2c1] 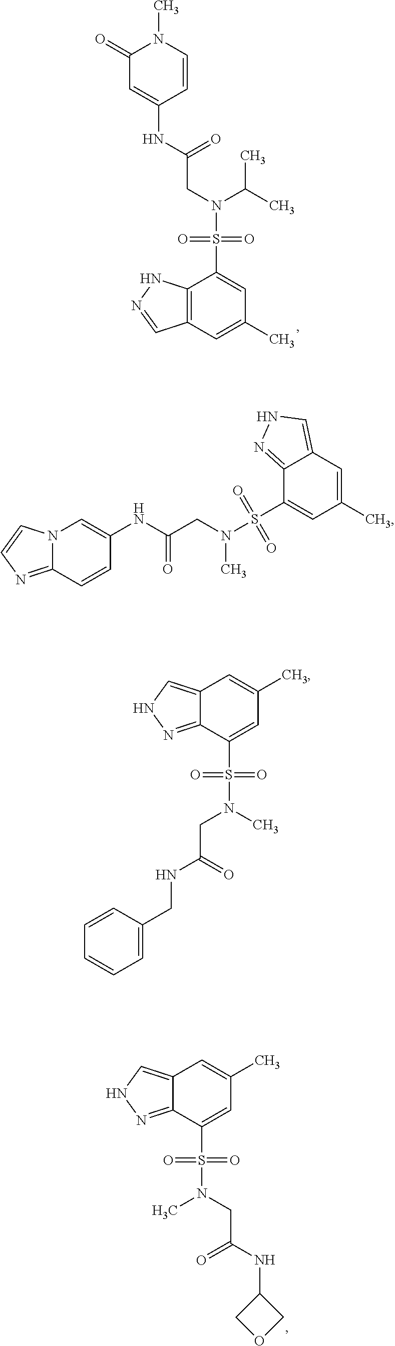 [SMILES: Cc1cc(S(=O)(=O)N(CC(=O)Nc2ccn(C)c(=O)c2)C(C)C)c2[nH]ncc2c1 ; Cc1cc(S(=O)(=O)N(C)CC(=O)Nc2ccc3nccn3c2)c2n[nH]cc2c1 ; Cc1cc(S(=O)(=O)N(C)CC(=O)NCc2ccccc2)c2n[nH]cc2c1 ; Cc1cc(S(=O)(=O)N(C)CC(=O)NC2COC2)c2n[nH]cc2c1] 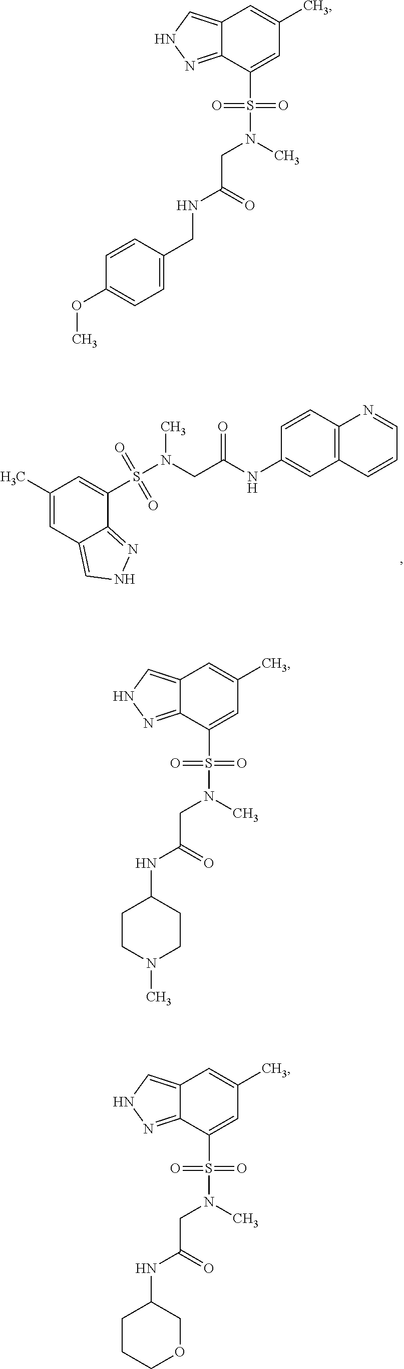 [SMILES: COc1ccc(CNC(=O)CN(C)S(=O)(=O)c2cc(C)cc3c[nH]nc23)cc1 ; Cc1cc(S(=O)(=O)N(C)CC(=O)Nc2ccc3ncccc3c2)c2n[nH]cc2c1 ; Cc1cc(S(=O)(=O)N(C)CC(=O)NC2CCN(C)CC2)c2n[nH]cc2c1 ; Cc1cc(S(=O)(=O)N(C)CC(=O)NC2CCCOC2)c2n[nH]cc2c1] 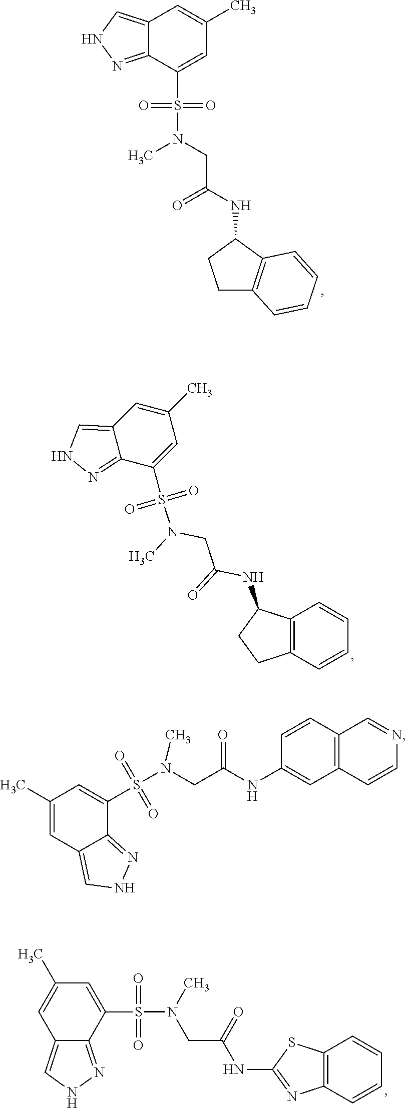 [SMILES: Cc1cc(S(=O)(=O)N(C)CC(=O)N[C@H]2CCc3ccccc32)c2n[nH]cc2c1 ; Cc1cc(S(=O)(=O)N(C)CC(=O)N[C@@H]2CCc3ccccc32)c2n[nH]cc2c1 ; Cc1cc(S(=O)(=O)N(C)CC(=O)Nc2ccc3cnccc3c2)c2n[nH]cc2c1 ; Cc1cc(S(=O)(=O)N(C)CC(=O)Nc2nc3ccccc3s2)c2n[nH]cc2c1] 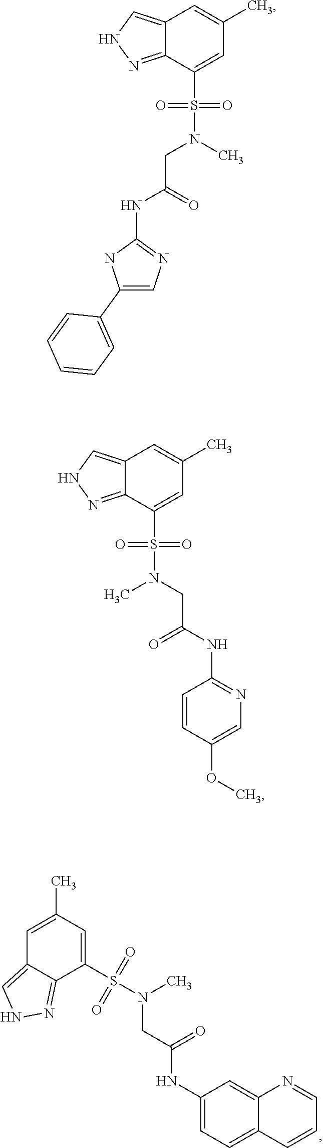 [SMILES: Cc1cc(S(=O)(=O)N(C)CC(=O)Nc2ncc(-c3ccccc3)[nH]2)c2n[nH]cc2c1 ; COc1ccc(NC(=O)CN(C)S(=O)(=O)c2cc(C)cc3c[nH]nc23)nc1 ; Cc1cc(S(=O)(=O)N(C)CC(=O)Nc2ccc3cccnc3c2)c2n[nH]cc2c1] 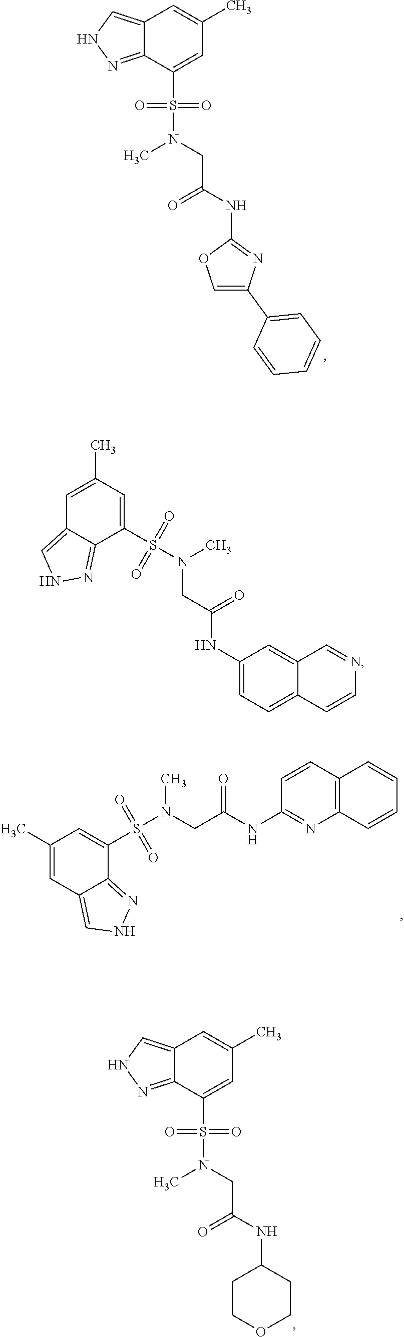 [SMILES: Cc1cc(S(=O)(=O)N(C)CC(=O)Nc2nc(-c3ccccc3)co2)c2n[nH]cc2c1 ; Cc1cc(S(=O)(=O)N(C)CC(=O)Nc2ccc3ccncc3c2)c2n[nH]cc2c1 ; Cc1cc(S(=O)(=O)N(C)CC(=O)Nc2ccc3ccccc3n2)c2n[nH]cc2c1 ; Cc1cc(S(=O)(=O)N(C)CC(=O)NC2CCOCC2)c2n[nH]cc2c1] 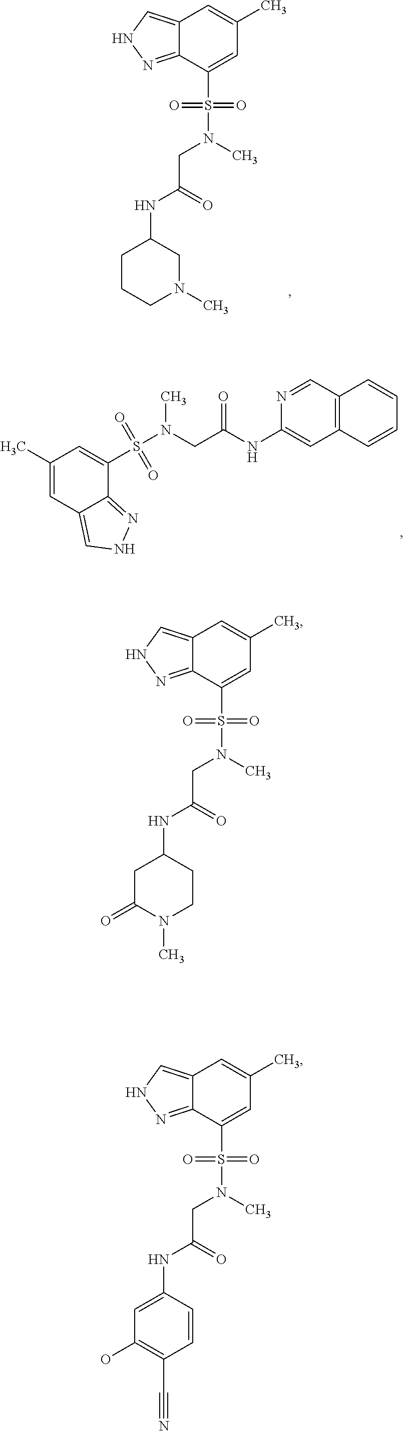 [SMILES: Cc1cc(S(=O)(=O)N(C)CC(=O)NC2CCCN(C)C2)c2n[nH]cc2c1 ; Cc1cc(S(=O)(=O)N(C)CC(=O)Nc2cc3ccccc3cn2)c2n[nH]cc2c1 ; Cc1cc(S(=O)(=O)N(C)CC(=O)NC2CCN(C)C(=O)C2)c2n[nH]cc2c1 ; Cc1cc(S(=O)(=O)N(C)CC(=O)Nc2ccc(C#N)c(O)c2)c2n[nH]cc2c1] 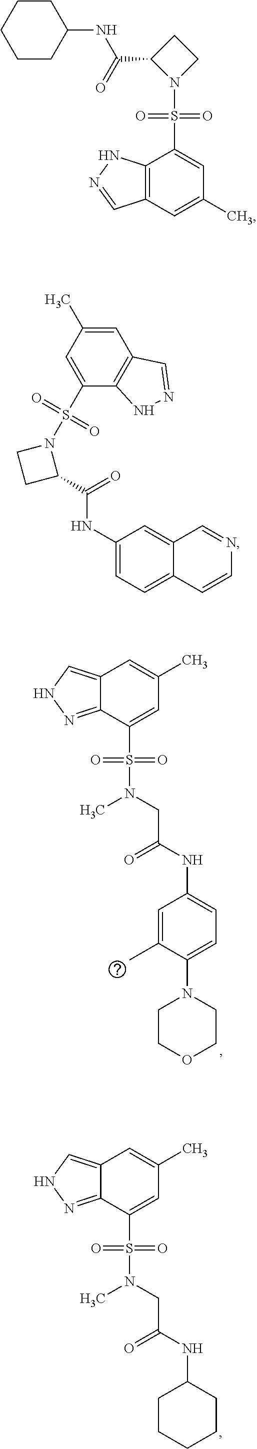 [SMILES: Cc1cc(S(=O)(=O)N2CC[C@H]2C(=O)NC2CCCCC2)c2[nH]ncc2c1 ; Cc1cc(S(=O)(=O)N2CC[C@H]2C(=O)Nc2ccc3ccncc3c2)c2[nH]ncc2c1 ; Cc1cc(S(=O)(=O)N(C)CC(=O)Nc2ccc(N3CCOCC3)c(C)c2)c2n[nH]cc2c1 ; Cc1cc(S(=O)(=O)N(C)CC(=O)NC2CCCCC2)c2n[nH]cc2c1] 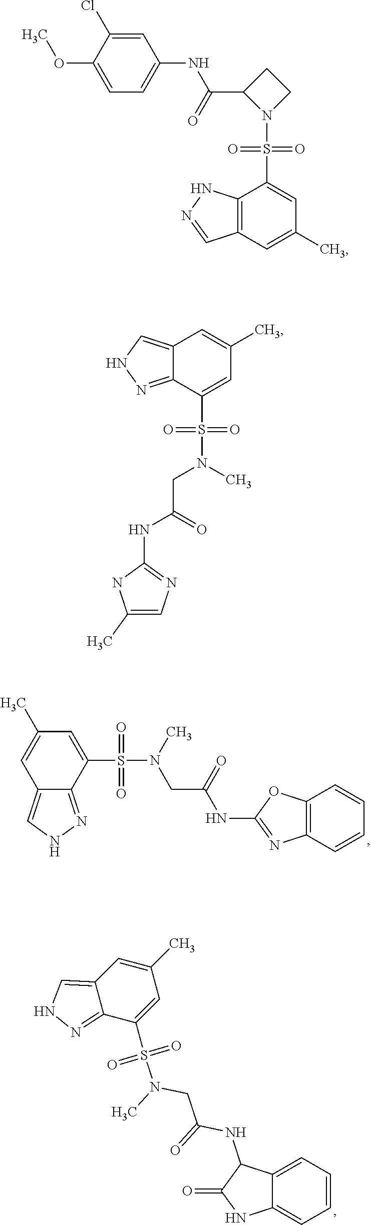 [SMILES: COc1ccc(NC(=O)C2CCN2S(=O)(=O)c2cc(C)cc3cn[nH]c23)cc1Cl ; Cc1cc(S(=O)(=O)N(C)CC(=O)Nc2ncc(C)[nH]2)c2n[nH]cc2c1 ; Cc1cc(S(=O)(=O)N(C)CC(=O)Nc2nc3ccccc3o2)c2n[nH]cc2c1 ; Cc1cc(S(=O)(=O)N(C)CC(=O)NC2C(=O)Nc3ccccc32)c2n[nH]cc2c1] 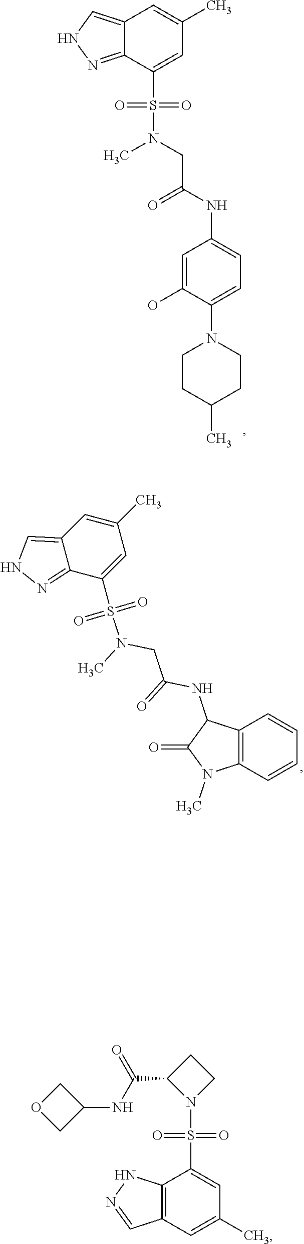 [SMILES: Cc1cc(S(=O)(=O)N(C)CC(=O)Nc2ccc(N3CCC(C)CC3)c(O)c2)c2n[nH]cc2c1 ; Cc1cc(S(=O)(=O)N(C)CC(=O)NC2C(=O)N(C)c3ccccc32)c2n[nH]cc2c1 ; Cc1cc(S(=O)(=O)N2CC[C@H]2C(=O)NC2COC2)c2[nH]ncc2c1] 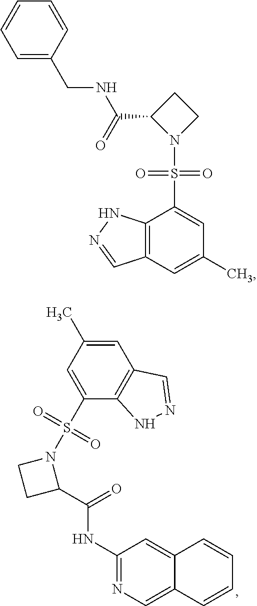 [SMILES: Cc1cc(S(=O)(=O)N2CC[C@H]2C(=O)NCc2ccccc2)c2[nH]ncc2c1 ; Cc1cc(S(=O)(=O)N2CCC2C(=O)Nc2cc3ccccc3cn2)c2[nH]ncc2c1] 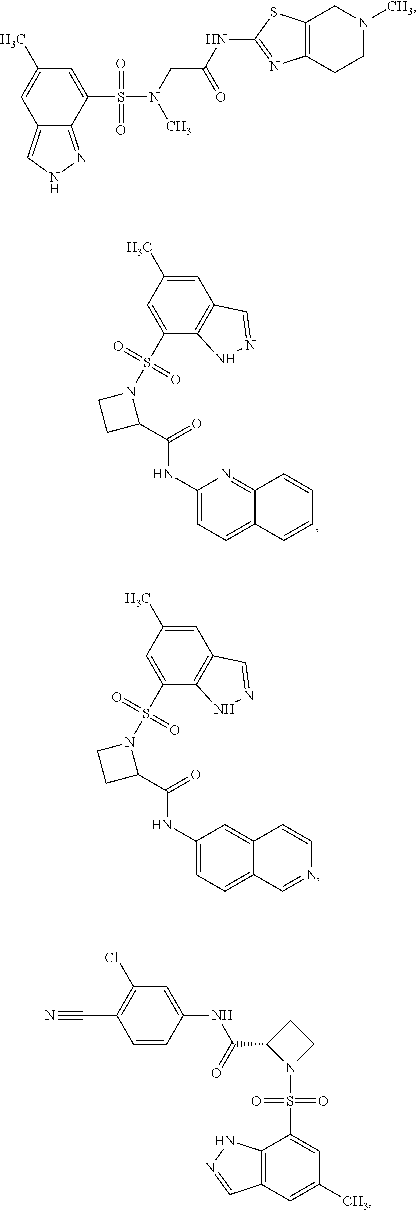 [SMILES: Cc1cc(S(=O)(=O)N(C)CC(=O)Nc2nc3c(s2)CN(C)CC3)c2n[nH]cc2c1 ; Cc1cc(S(=O)(=O)N2CCC2C(=O)Nc2ccc3ccccc3n2)c2[nH]ncc2c1 ; Cc1cc(S(=O)(=O)N2CCC2C(=O)Nc2ccc3cnccc3c2)c2[nH]ncc2c1 ; Cc1cc(S(=O)(=O)N2CC[C@H]2C(=O)Nc2ccc(C#N)c(Cl)c2)c2[nH]ncc2c1] 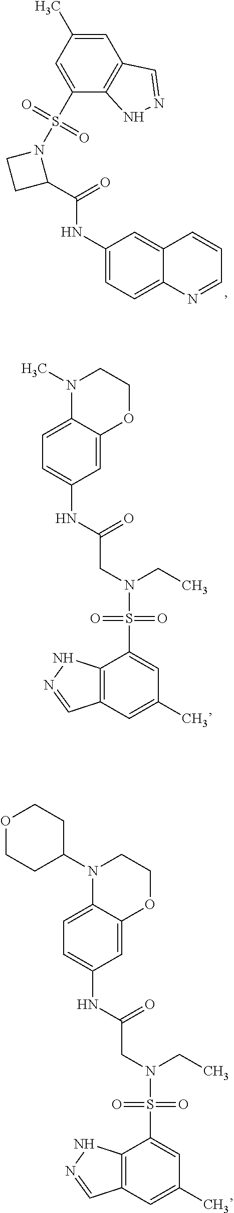 [SMILES: Cc1cc(S(=O)(=O)N2CCC2C(=O)Nc2ccc3ncccc3c2)c2[nH]ncc2c1 ; CCN(CC(=O)Nc1ccc2c(c1)OCCN2C)S(=O)(=O)c1cc(C)cc2cn[nH]c12 ; CCN(CC(=O)Nc1ccc2c(c1)OCCN2C1CCOCC1)S(=O)(=O)c1cc(C)cc2cn[nH]c12] 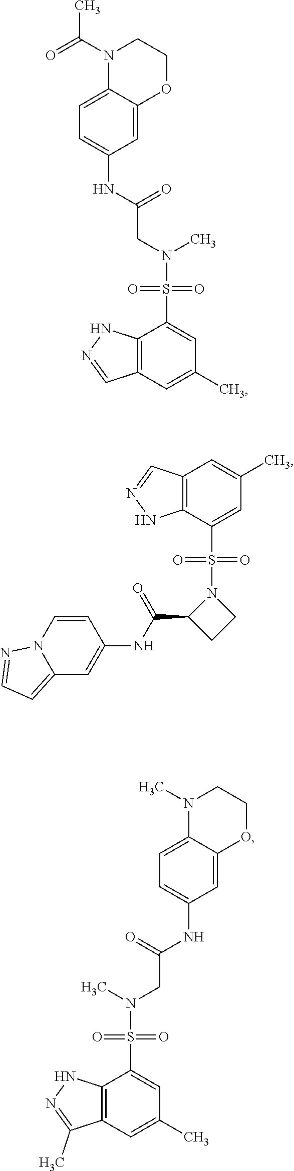 [SMILES: CC(=O)N1CCOc2cc(NC(=O)CN(C)S(=O)(=O)c3cc(C)cc4cn[nH]c34)ccc21 ; Cc1cc(S(=O)(=O)N2CC[C@H]2C(=O)Nc2ccn3nccc3c2)c2[nH]ncc2c1 ; Cc1cc(S(=O)(=O)N(C)CC(=O)Nc2ccc3c(c2)OCCN3C)c2[nH]nc(C)c2c1] 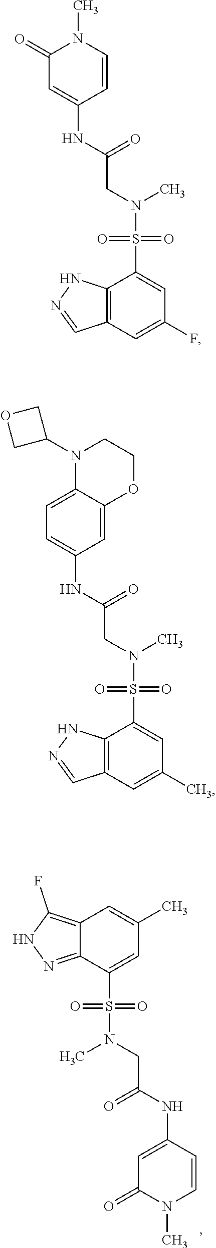 [SMILES: CN(CC(=O)Nc1ccn(C)c(=O)c1)S(=O)(=O)c1cc(F)cc2cn[nH]c12 ; Cc1cc(S(=O)(=O)N(C)CC(=O)Nc2ccc3c(c2)OCCN3C2COC2)c2[nH]ncc2c1 ; Cc1cc(S(=O)(=O)N(C)CC(=O)Nc2ccn(C)c(=O)c2)c2n[nH]c(F)c2c1] 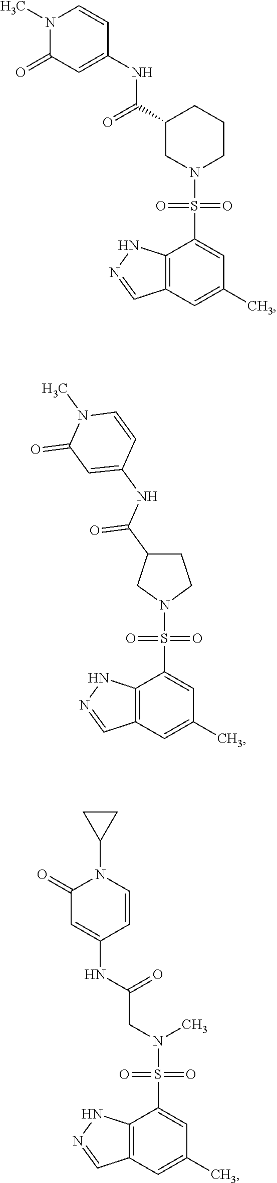 [SMILES: Cc1cc(S(=O)(=O)N2CCC[C@@H](C(=O)Nc3ccn(C)c(=O)c3)C2)c2[nH]ncc2c1 ; Cc1cc(S(=O)(=O)N2CCC(C(=O)Nc3ccn(C)c(=O)c3)C2)c2[nH]ncc2c1 ; Cc1cc(S(=O)(=O)N(C)CC(=O)Nc2ccn(C3CC3)c(=O)c2)c2[nH]ncc2c1] 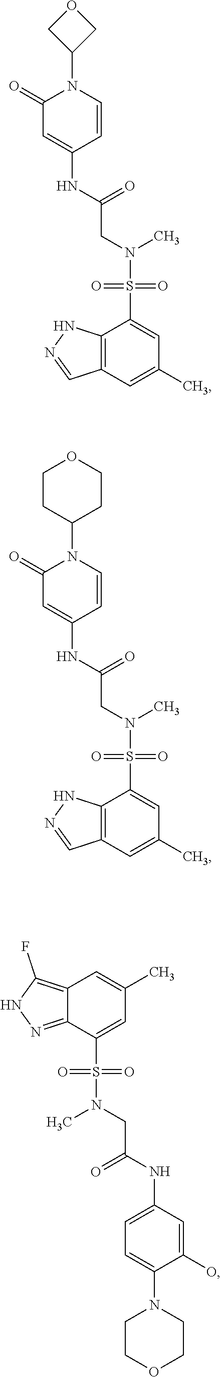 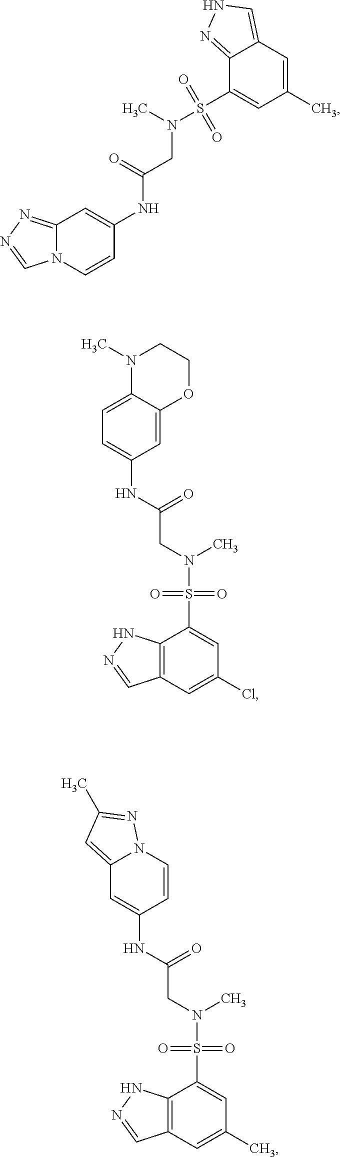 [SMILES: Cc1cc(S(=O)(=O)N(C)CC(=O)Nc2ccn3cnnc3c2)c2n[nH]cc2c1 ; CN1CCOc2cc(NC(=O)CN(C)S(=O)(=O)c3cc(Cl)cc4cn[nH]c34)ccc21 ; Cc1cc(S(=O)(=O)N(C)CC(=O)Nc2ccn3nc(C)cc3c2)c2[nH]ncc2c1] 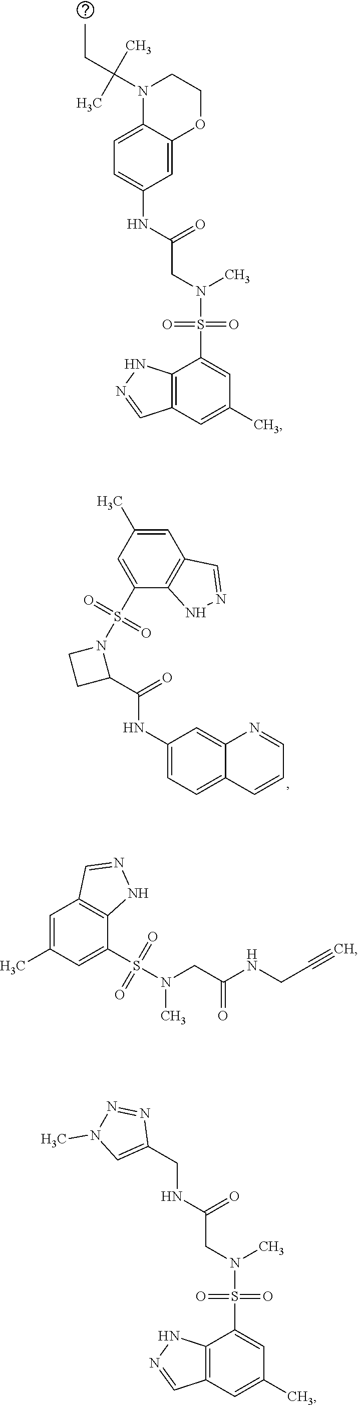 [SMILES: CCC(C)(C)N1CCOc2cc(NC(=O)CN(C)S(=O)(=O)c3cc(C)cc4cn[nH]c34)ccc21 ; Cc1cc(S(=O)(=O)N2CCC2C(=O)Nc2ccc3cccnc3c2)c2[nH]ncc2c1 ; C#CCNC(=O)CN(C)S(=O)(=O)c1cc(C)cc2cn[nH]c12 ; Cc1cc(S(=O)(=O)N(C)CC(=O)NCc2cn(C)nn2)c2[nH]ncc2c1] 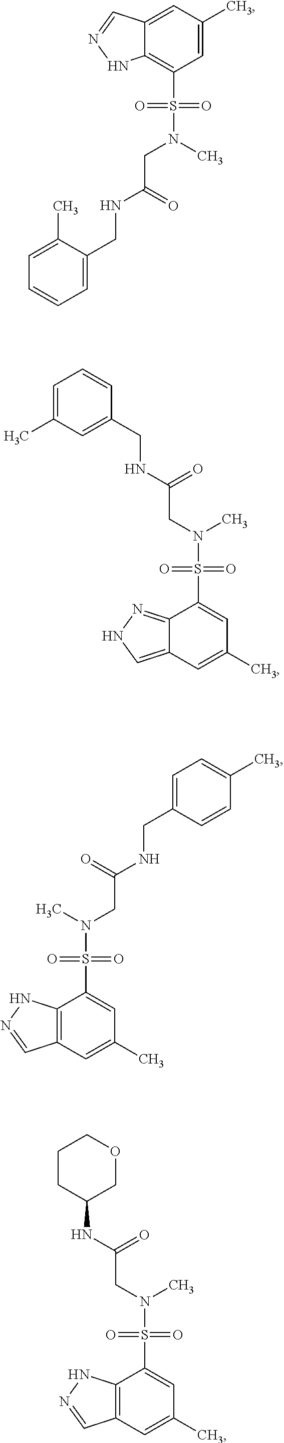 [SMILES: Cc1cc(S(=O)(=O)N(C)CC(=O)NCc2ccccc2C)c2[nH]ncc2c1 ; Cc1cccc(CNC(=O)CN(C)S(=O)(=O)c2cc(C)cc3c[nH]nc23)c1 ; Cc1ccc(CNC(=O)CN(C)S(=O)(=O)c2cc(C)cc3cn[nH]c23)cc1 ; Cc1cc(S(=O)(=O)N(C)CC(=O)N[C@H]2CCCOC2)c2[nH]ncc2c1] 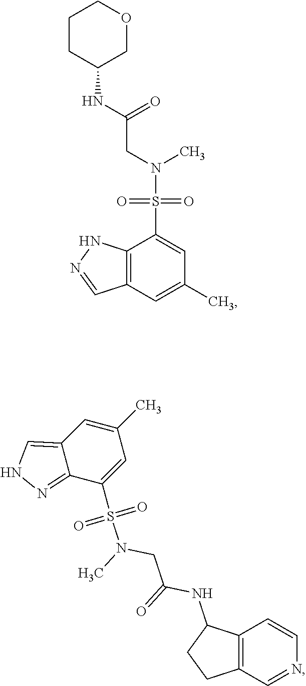 [SMILES: Cc1cc(S(=O)(=O)N(C)CC(=O)N[C@@H]2CCCOC2)c2[nH]ncc2c1 ; Cc1cc(S(=O)(=O)N(C)CC(=O)NC2CCc3cnccc32)c2n[nH]cc2c1] 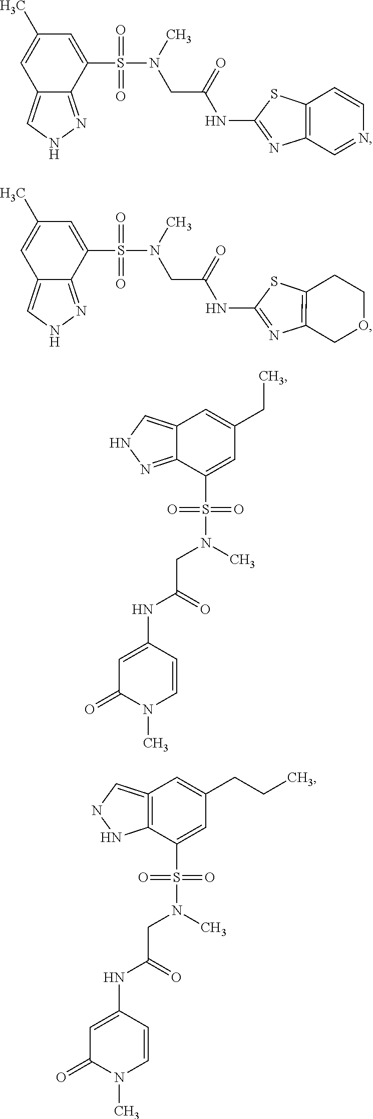 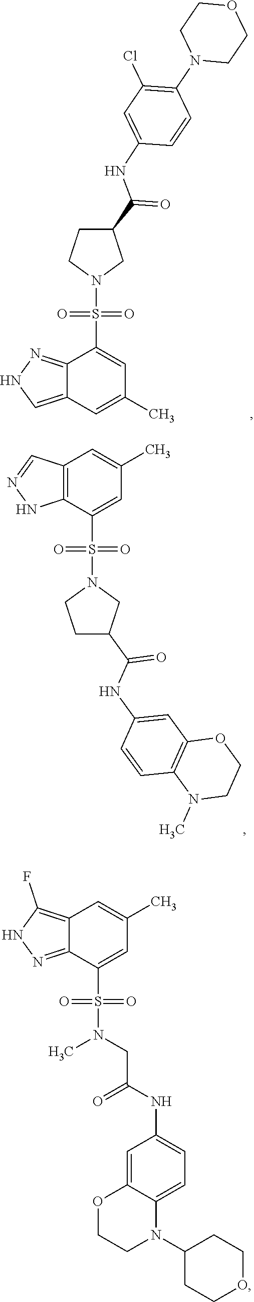 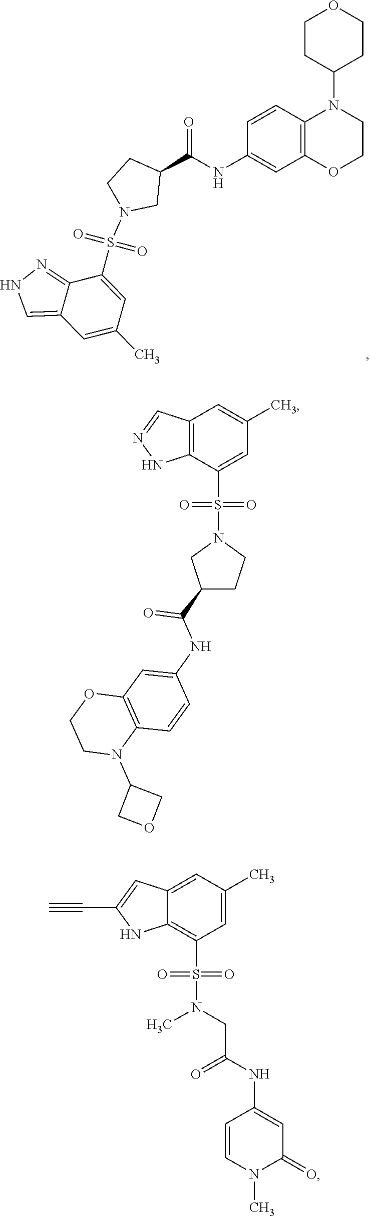 [SMILES: Cc1cc(S(=O)(=O)N2CC[C@@H](C(=O)Nc3ccc4c(c3)OCCN4C3CCOCC3)C2)c2n[nH]cc2c1 ; Cc1cc(S(=O)(=O)N2CC[C@@H](C(=O)Nc3ccc4c(c3)OCCN4C3COC3)C2)c2[nH]ncc2c1 ; C#Cc1cc2cc(C)cc(S(=O)(=O)N(C)CC(=O)Nc3ccn(C)c(=O)c3)c2[nH]1] 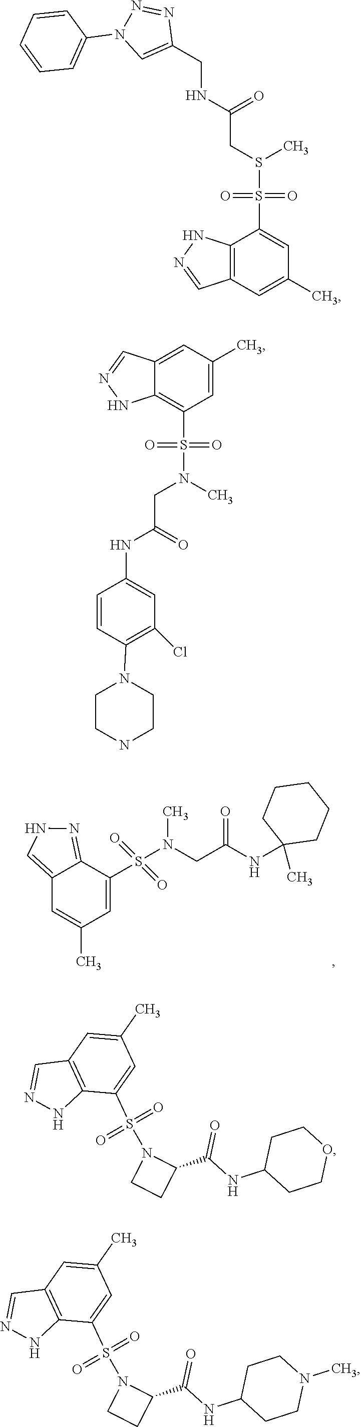 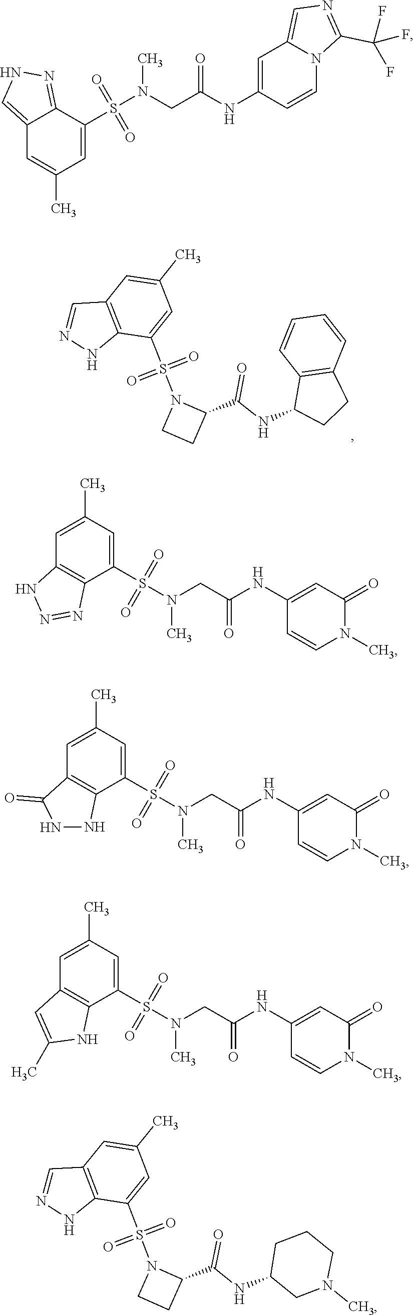 [SMILES: Cc1cc(S(=O)(=O)N(C)CC(=O)Nc2ccn3c(C(F)(F)F)ncc3c2)c2n[nH]cc2c1 ; Cc1cc(S(=O)(=O)N2CC[C@H]2C(=O)N[C@H]2CCc3ccccc32)c2[nH]ncc2c1 ; Cc1cc(S(=O)(=O)N(C)CC(=O)Nc2ccn(C)c(=O)c2)c2nn[nH]c2c1 ; Cc1cc(S(=O)(=O)N(C)CC(=O)Nc2ccn(C)c(=O)c2)c2[nH][nH]c(=O)c2c1 ; Cc1cc(S(=O)(=O)N(C)CC(=O)Nc2ccn(C)c(=O)c2)c2[nH]c(C)cc2c1 ; Cc1cc(S(=O)(=O)N2CC[C@H]2C(=O)N[C@@H]2CCCN(C)C2)c2[nH]ncc2c1] 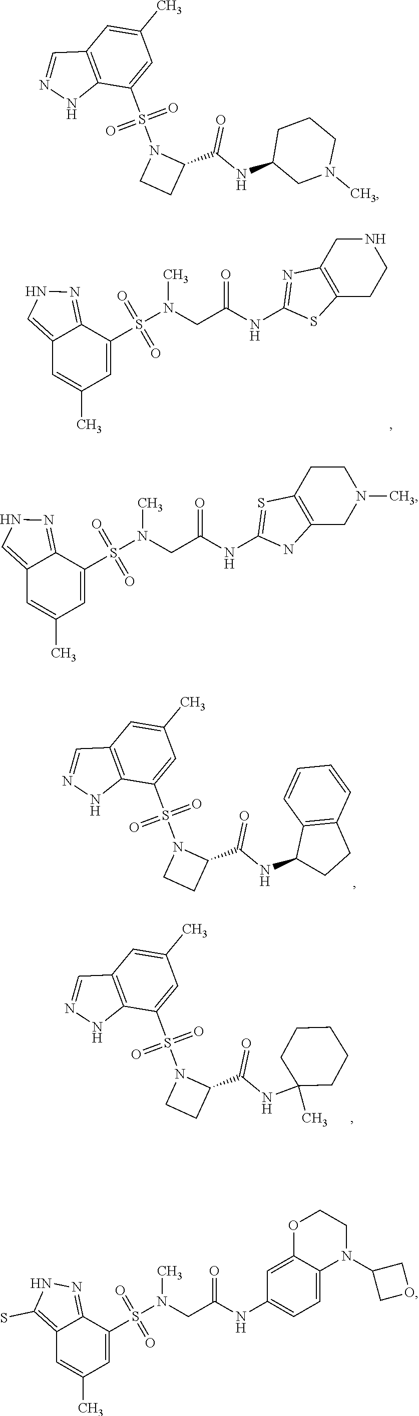 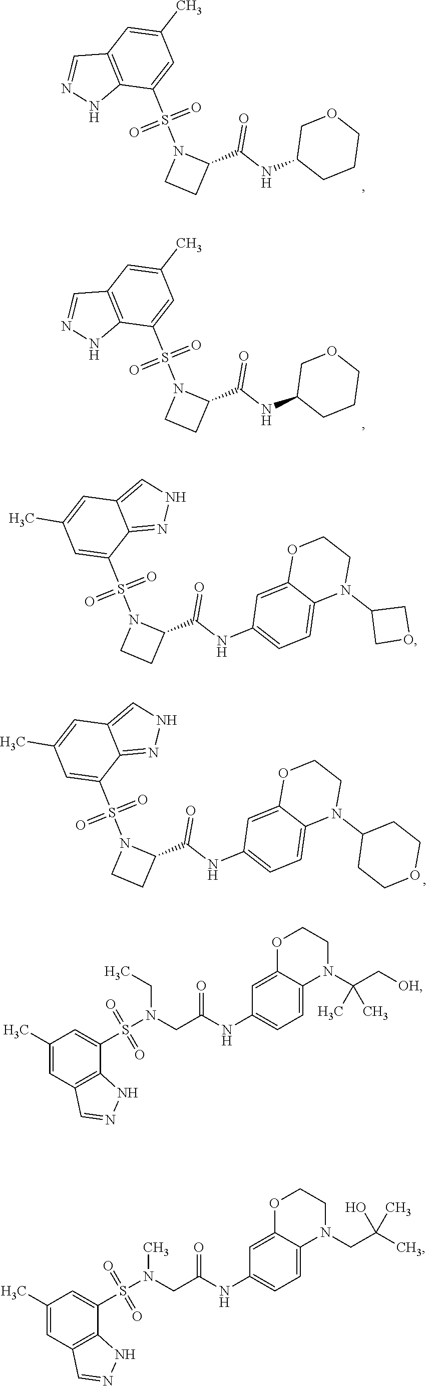 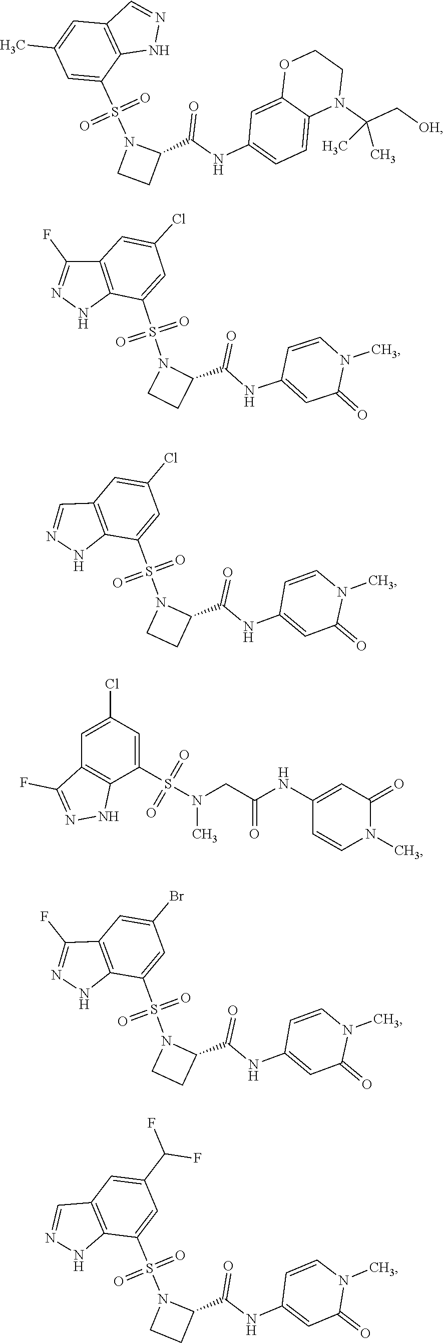 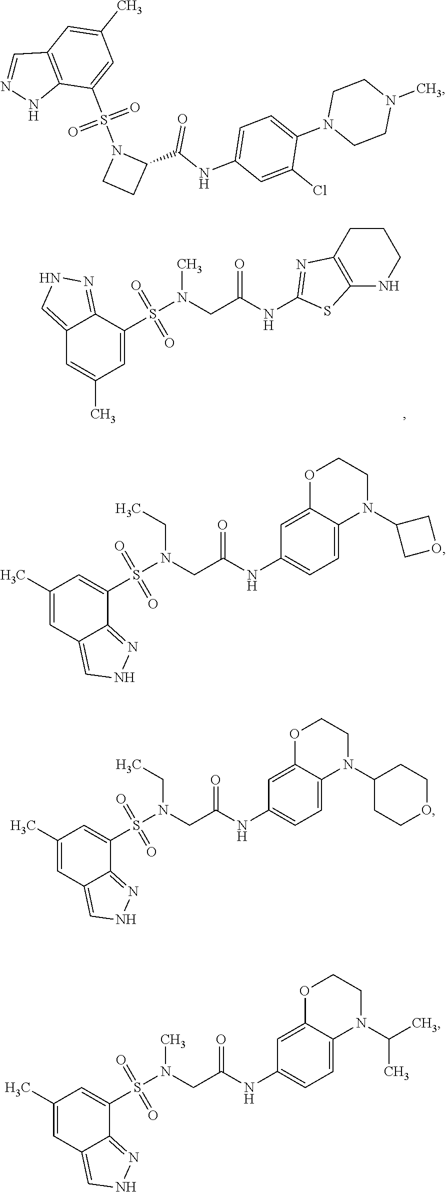 [SMILES: Cc1cc(S(=O)(=O)N2CC[C@H]2C(=O)Nc2ccc(N3CCN(C)CC3)c(Cl)c2)c2[nH]ncc2c1 ; Cc1cc(S(=O)(=O)N(C)CC(=O)Nc2nc3c(s2)NCCC3)c2n[nH]cc2c1 ; CCN(CC(=O)Nc1ccc2c(c1)OCCN2C1COC1)S(=O)(=O)c1cc(C)cc2c[nH]nc12 ; CCN(CC(=O)Nc1ccc2c(c1)OCCN2C1CCOCC1)S(=O)(=O)c1cc(C)cc2c[nH]nc12 ; Cc1cc(S(=O)(=O)N(C)CC(=O)Nc2ccc3c(c2)OCCN3C(C)C)c2n[nH]cc2c1] 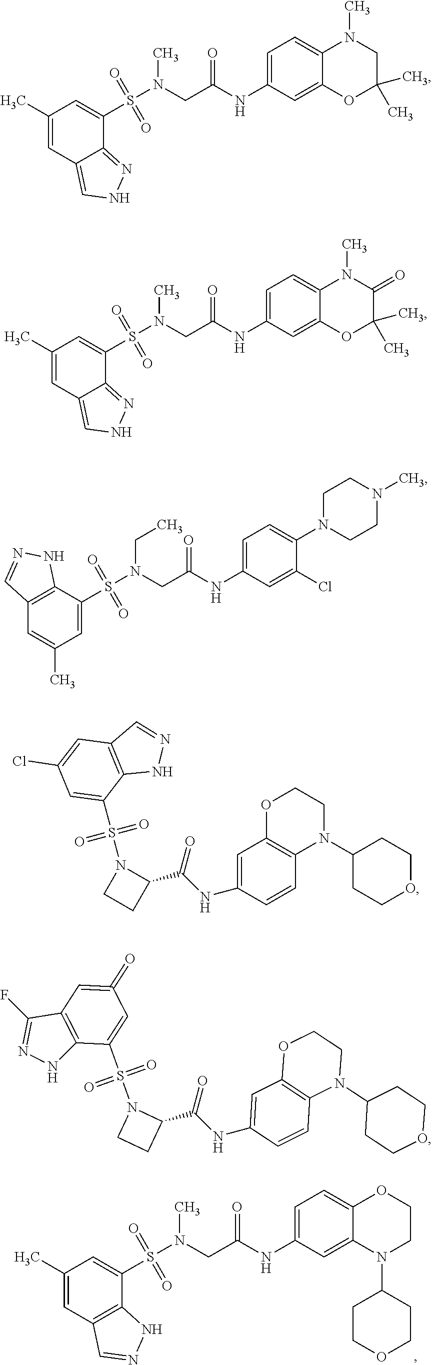 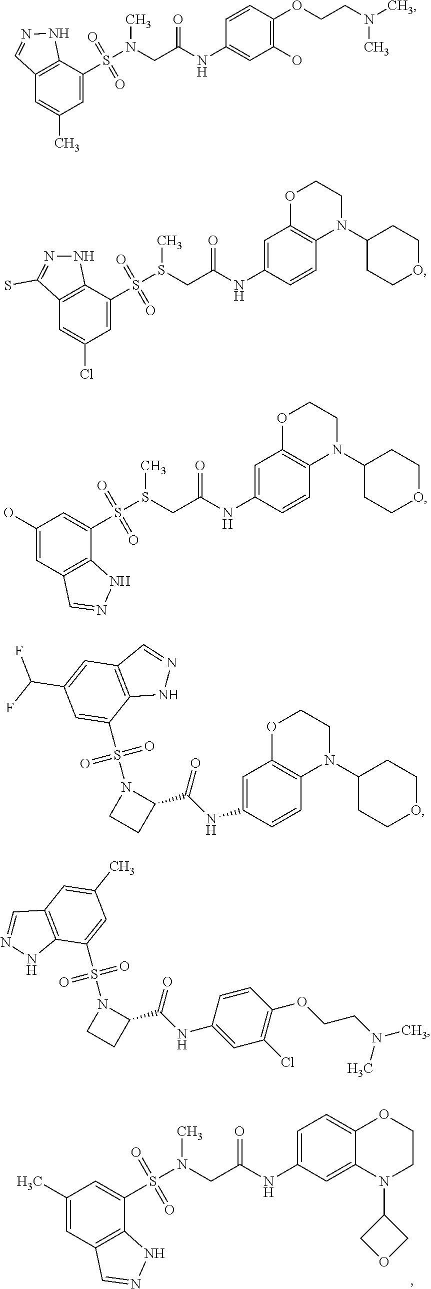 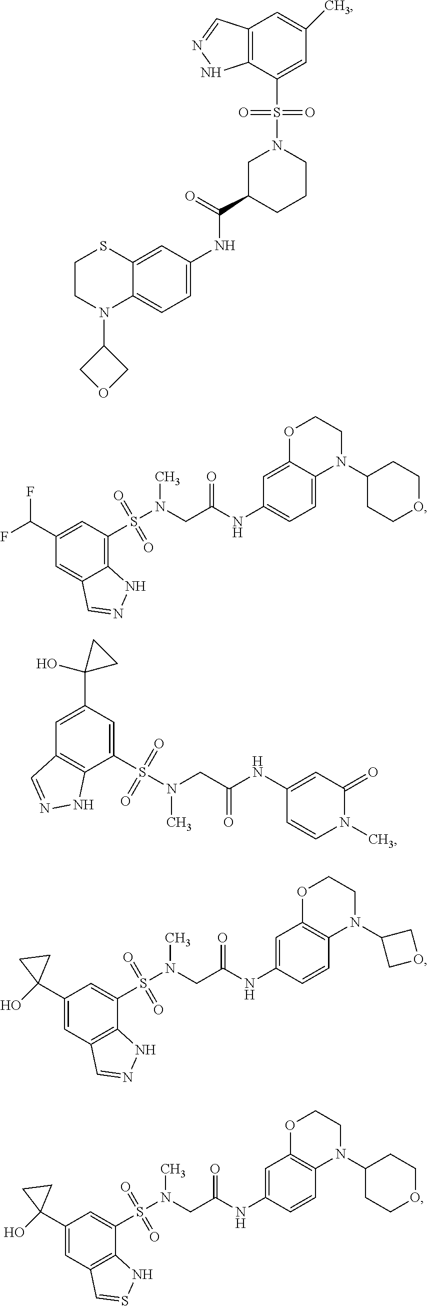 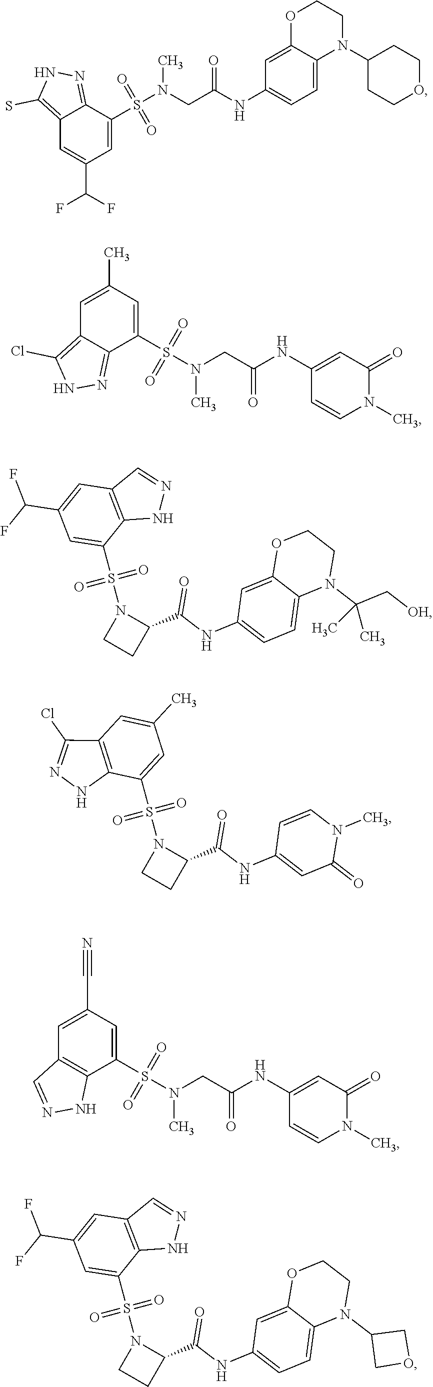 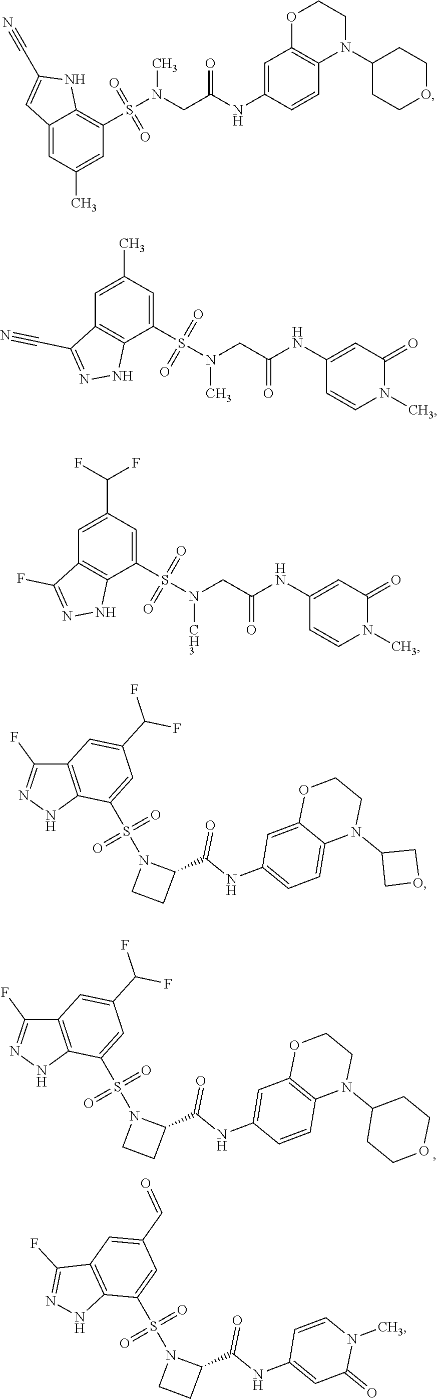 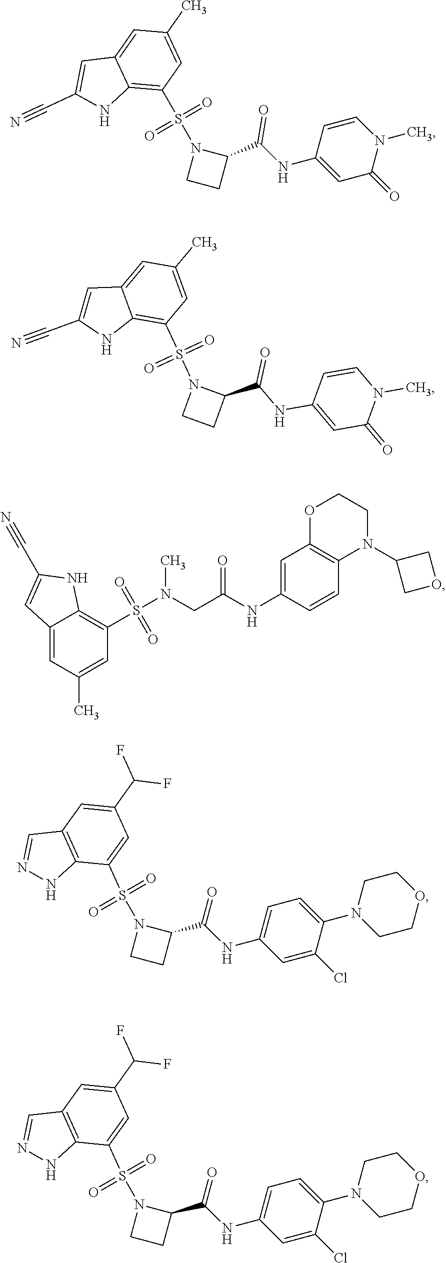 [SMILES: Cc1cc(S(=O)(=O)N2CC[C@H]2C(=O)Nc2ccn(C)c(=O)c2)c2[nH]c(C#N)cc2c1 ; Cc1cc(S(=O)(=O)N2CC[C@@H]2C(=O)Nc2ccn(C)c(=O)c2)c2[nH]c(C#N)cc2c1 ; Cc1cc(S(=O)(=O)N(C)CC(=O)Nc2ccc3c(c2)OCCN3C2COC2)c2[nH]c(C#N)cc2c1 ; O=C(Nc1ccc(N2CCOCC2)c(Cl)c1)[C@@H]1CCN1S(=O)(=O)c1cc(C(F)F)cc2cn[nH]c12 ; O=C(Nc1ccc(N2CCOCC2)c(Cl)c1)[C@H]1CCN1S(=O)(=O)c1cc(C(F)F)cc2cn[nH]c12] 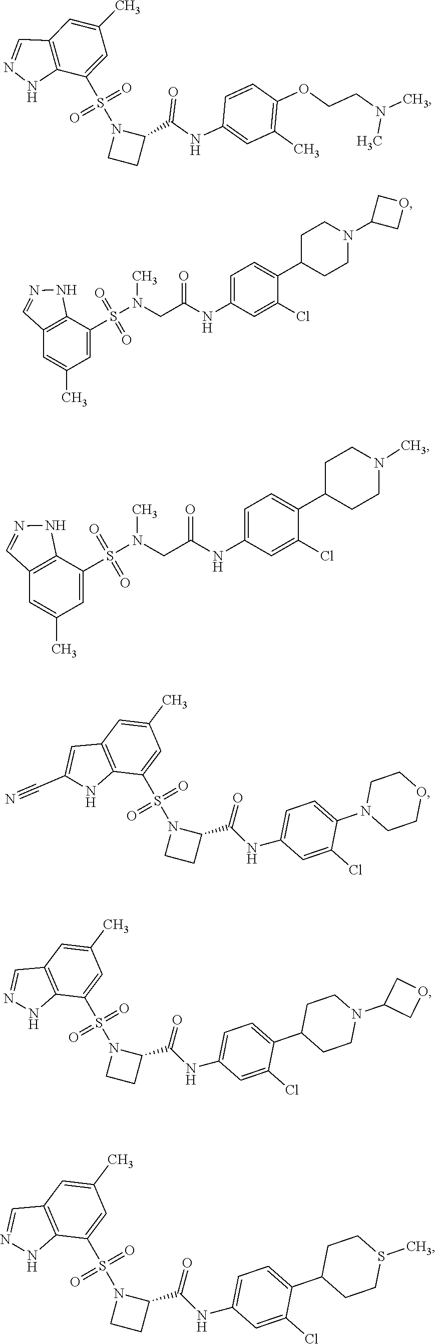 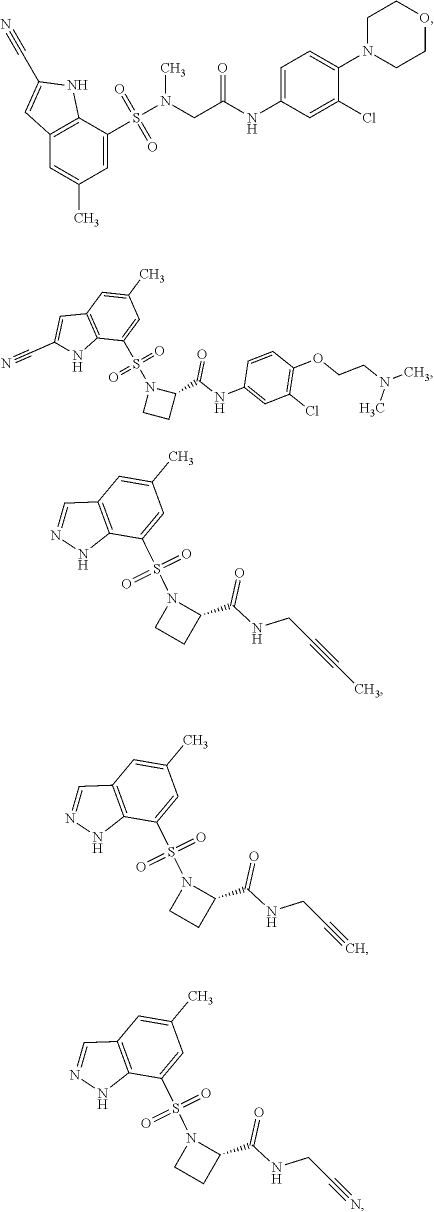 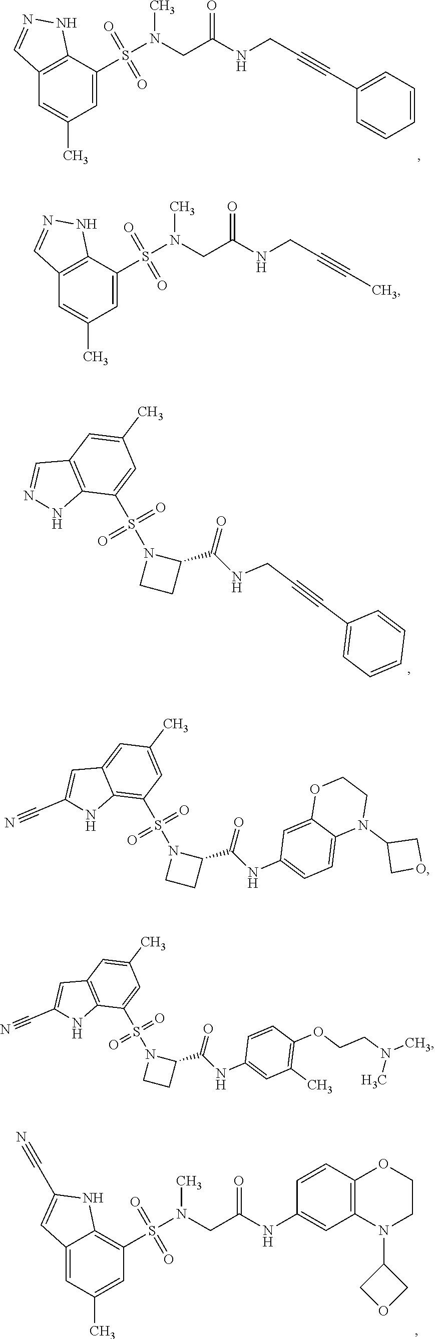 [SMILES: Cc1cc(S(=O)(=O)N(C)CC(=O)NCC#Cc2ccccc2)c2[nH]ncc2c1 ; CC#CCNC(=O)CN(C)S(=O)(=O)c1cc(C)cc2cn[nH]c12 ; Cc1cc(S(=O)(=O)N2CC[C@H]2C(=O)NCC#Cc2ccccc2)c2[nH]ncc2c1 ; Cc1cc(S(=O)(=O)N2CC[C@H]2C(=O)Nc2ccc3c(c2)OCCN3C2COC2)c2[nH]c(C#N)cc2c1 ; Cc1cc(S(=O)(=O)N2CC[C@H]2C(=O)Nc2ccc(OCCN(C)C)c(C)c2)c2[nH]c(C#N)cc2c1 ; Cc1cc(S(=O)(=O)N(C)CC(=O)Nc2ccc3c(c2)N(C2COC2)CCO3)c2[nH]c(C#N)cc2c1] 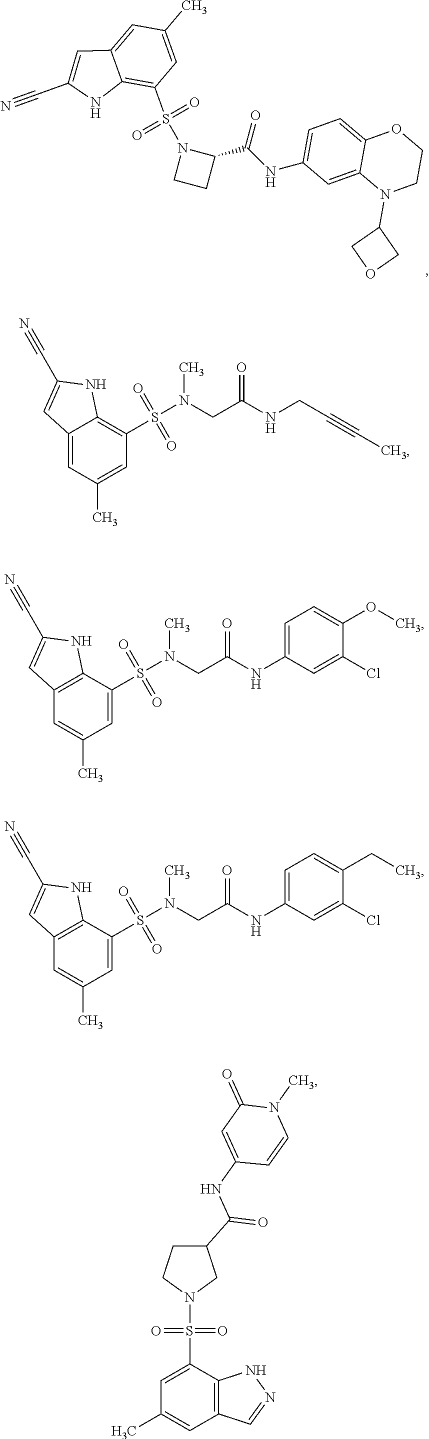 [SMILES: Cc1cc(S(=O)(=O)N2CC[C@H]2C(=O)Nc2ccc3c(c2)N(C2COC2)CCO3)c2[nH]c(C#N)cc2c1 ; CC#CCNC(=O)CN(C)S(=O)(=O)c1cc(C)cc2cc(C#N)[nH]c12 ; COc1ccc(NC(=O)CN(C)S(=O)(=O)c2cc(C)cc3cc(C#N)[nH]c23)cc1Cl ; CCc1ccc(NC(=O)CN(C)S(=O)(=O)c2cc(C)cc3cc(C#N)[nH]c23)cc1Cl ; Cc1cc(S(=O)(=O)N2CCC(C(=O)Nc3ccn(C)c(=O)c3)C2)c2[nH]ncc2c1] 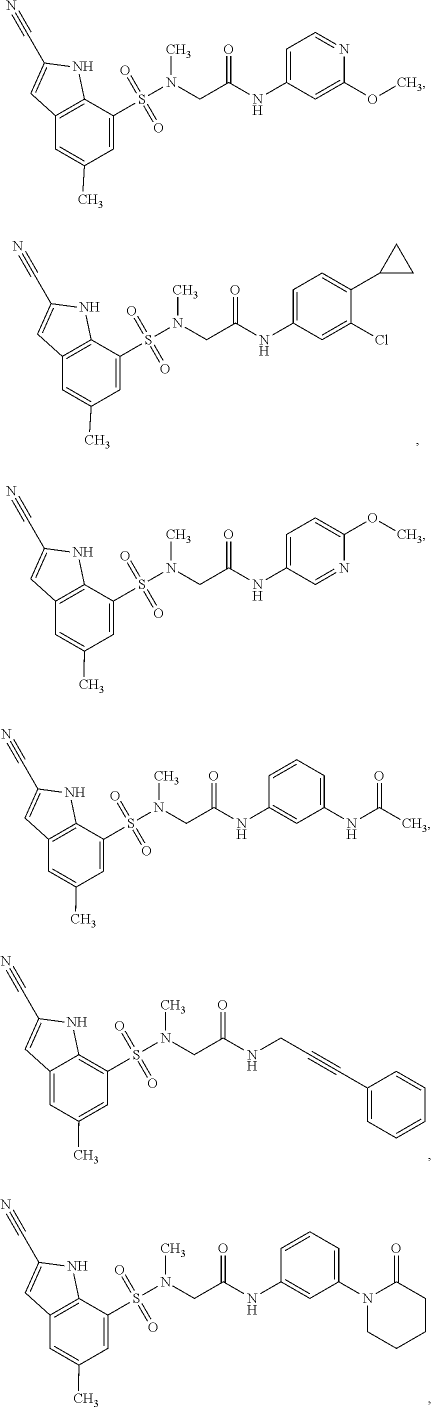 [SMILES: COc1cc(NC(=O)CN(C)S(=O)(=O)c2cc(C)cc3cc(C#N)[nH]c23)ccn1 ; Cc1cc(S(=O)(=O)N(C)CC(=O)Nc2ccc(C3CC3)c(Cl)c2)c2[nH]c(C#N)cc2c1 ; COc1ccc(NC(=O)CN(C)S(=O)(=O)c2cc(C)cc3cc(C#N)[nH]c23)cn1 ; CC(=O)Nc1cccc(NC(=O)CN(C)S(=O)(=O)c2cc(C)cc3cc(C#N)[nH]c23)c1 ; Cc1cc(S(=O)(=O)N(C)CC(=O)NCC#Cc2ccccc2)c2[nH]c(C#N)cc2c1 ; Cc1cc(S(=O)(=O)N(C)CC(=O)Nc2cccc(N3CCCCC3=O)c2)c2[nH]c(C#N)cc2c1] 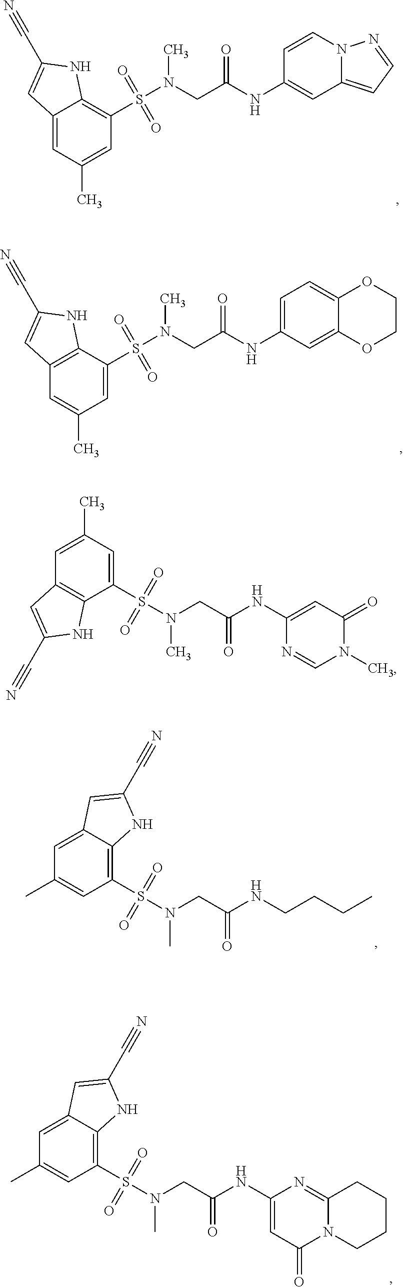 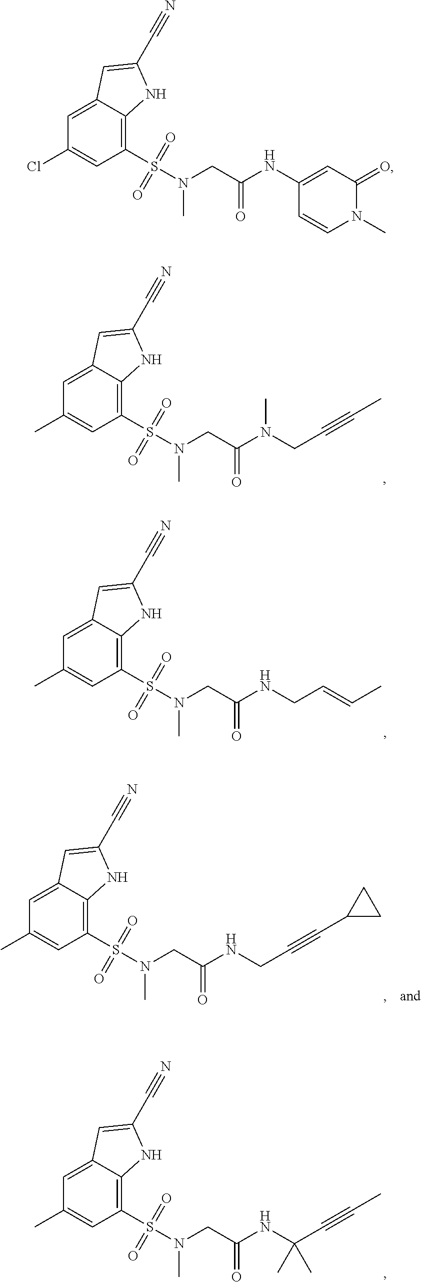
or a pharmaceutically acceptable salt thereof.

48. A pharmaceutical composition, comprising a compound of any one of claims 1-47 and a pharmaceutically acceptable carrier.

49. A method of inhibiting or killing Mycobacterium tuberculosis in vitro, comprising contacting Mycobacterium tuberculosis with a compound of any one of claims 1-47.

50. A method of treating tuberculosis, comprising administering to a subject in need thereof a compound of any one of claims 1-47.

51. The method of claim 50, wherein the subject is a mammal.

52. The method of claim 50, wherein the subject is a human.
